(19) United States
(12) Patent Application Publication
(10) Pub. No.: US 2024/0206325 A1
(43) Pub. Date: Jun. 20, 2024

(54) COMPOUND AND ORGANIC ELECTROLUMINESCENCE DEVICE

(71) Applicant: IDEMITSU KOSAN CO.,LTD., Tokyo (JP)

(72) Inventors: Yoshinao SHIRASAKI, Tokyo (JP); Kazuma MASE, Tokyo (JP); Hiroaki ITOI, Tokyo (JP)

(73) Assignee: IDEMITSU KOSAN CO.,LTD., Tokyo (JP)

(21) Appl. No.: 18/281,297

(22) PCT Filed: Mar. 10, 2022
(86) PCT No.: PCT/JP2022/010552

§ 371 Date: Sep. 10, 2023

(30) Foreign Application Priority Data
Mar. 11, 2021 (JP) 2021-039101

(51) Int. Cl. H10K 85/60; C07D 307/77; C07D 487/22; C09K 11/06

(52) U.S. Cl. CPC H10K 85/633; C07D 307/77; C07D 487/22; C09K 11/06; H10K 85/636; H10K 50/12

(57) ABSTRACT

A compound represented by the following formula (1), wherein in the formula (1), A is a substituted or unsubstituted aromatic hydrocarbon ring group containing a nitrogen atom as a part of a substituent and having a fused ring composed of 5 or more rings, or a substituted or unsubstituted heterocyclic group containing a nitrogen atom as a part of the skeleton or a substituent and having a fused ring composed of 5 or more rings; B is a monovalent group represented by the following formula (b1); wherein in the formula (b1), *B is bonded with the nitrogen atom contained as a part of the skeleton of A or a substituent via a single bond.

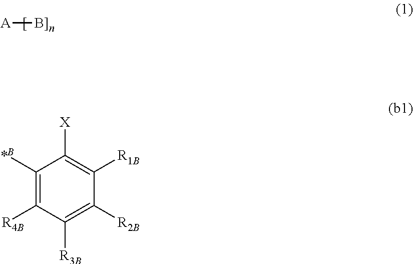

### TECHNICAL FIELD

The present invention relates to a novel compound and an organic electroluminescence device.

### BACKGROUND ART

When voltage is applied to an organic electroluminescence device (hereinafter, also referred to as an organic EL device), holes and electrons are injected into an emitting layer from an anode and a cathode, respectively. Then, thus injected holes and electrons are recombined in the emitting layer, and excitons are formed therein.

Conventional organic EL devices have not yet had sufficient device performance. Although materials used for the organic EL device are gradually improved to enhance the device performance, further performance enhancement is required.

Patent Document 1 discloses that a compound having the specific structure is used for an emitting layer of an organic EL device.

### RELATED ART DOCUMENTS

### Patent Documents

[Patent Document 1] WO 2017/023021 A1

### Summary of the Invention

When the emission wavelength from an organic EL device is shortened, apparent efficiency thereof is decreased by decreasing visibility thereof. Further, it has been concerned that one's photoreceptor cell is adversely affected by enhancing emission energy.

It is an object of the present invention to provide a compound capable of fabricating an organic EL device having high chromatic purity and long lifetime without shortening emission wavelength therefrom.

As a result of intensive studies to achieve the above object, the present inventors have found that an organic EL device having high chromatic purity and long lifetime without shortening emission wavelength therefrom can be obtained by using a compound having the specific structure, and have completed the present invention.

According to the present invention, the following compound and the like are provided.

A compound represented by the following formula (1):

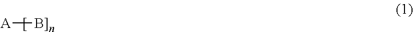

wherein in the formula (1),
A is
a substituted or unsubstituted aromatic hydrocarbon ring group containing a nitrogen atom as a part of a substituent and having a fused ring composed of 5 or more rings, or
a substituted or unsubstituted heterocyclic group containing a nitrogen atom as a part of the skeleton or a substituent and having a fused ring composed of 5 or more rings;
B is a monovalent group represented by the following formula (b1):

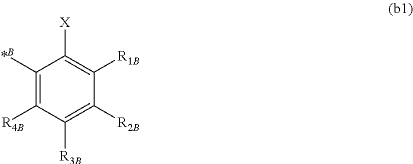

wherein in the formula (b1),
*B is bonded with the nitrogen atom contained as a part of the skeleton of A or a substituent via a single bond;
one or more sets of the adjacent two or more of R_{1B} to R_{4B} form an unsubstituted saturated ring, or a substituted or unsubstituted, unsaturated ring by bonding with each other;
R_{1B} to R_{4B} which do not form the unsubstituted saturated ring, or the substituted or unsubstituted, unsaturated ring are independently a hydrogen atom or a substituent R;
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₀)(R₉₀₂)(R₉₀₃),
—O—(R₉₀₄),
—S—(R₉₀₅),
—N(R₉₀₆)(R₉₀₇)
(wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms;
when two or more substituents R are present, the two or more substituents R may be the same as or different from each other;
X is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₀)(R₉₀₂)(R₉₀₃),
—O—(R₉₀₄),
—S—(R₉₀₅),
—N(R₉₀₆)(R₉₀₇),
a halogen atom, a cyano group, a nitro group, and
a substituted or unsubstituted aryl group having 6 to 50 ring carbon atoms;
R₉₀₁ to R₉₀₇ are the same as defined in the substituent R; and
n is an integer of 1 to 5; when n is 2 or more, two or more B's may be the same as or different from each other.

According to the present invention, there can be provided an organic EL device having high chromatic purity and long lifetime without shortening emission wavelength therefrom.

### BRIEF DESCRIPTION OF THE DRAWINGS

FIG. 1 is a diagram showing a schematic configuration of an organic EL device according to an aspect of the present invention.

### MODE FOR CARRYING OUT THE INVENTION

[Definition]In this specification, a hydrogen atom includes its isotopes different in the number of neutrons, namely, a protium, a deuterium and a tritium.

In this specification, at a bondable position in a chemical formula where a symbol such as “R”, or “D” representing a deuterium atom is not indicated, a hydrogen atom, that is, a protium atom, a deuterium atom or a tritium atom is bonded.

In this specification, the number of ring carbon atoms represents the number of carbon atoms forming a subject ring itself among the carbon atoms of a compound having a structure in which atoms are bonded in a ring form (for example, a monocyclic compound, a fused ring compound, a cross-linked compound, a carbocyclic compound, or a heterocyclic compound). When the subject ring is substituted by a substituent, the carbon contained in the substituent is not included in the number of ring carbon atoms. The same shall apply to “the number of ring carbon atoms” described below, unless otherwise specified. For example, a benzene ring has 6 ring carbon atoms, a naphthalene ring includes 10 ring carbon atoms, a pyridine ring includes 5 ring carbon atoms, and a furan ring includes 4 ring carbon atoms. Further, for example, a 9,9-diphenylfluorenyl group includes 13 ring carbon atoms, and a 9,9′-spirobifluorenyl group includes 25 ring carbon atoms.

When a benzene ring is substituted by, for example, an alkyl group as a substituent, the number of carbon atoms of the alkyl group is not included in the number of ring carbon atoms of the benzene ring. Therefore, the number of ring carbon atoms of the benzene ring substituted by the alkyl group is 6. When a naphthalene ring is substituted by, for example, an alkyl group as a substituent, the number of carbon atoms of the alkyl group is not included in the number of ring carbon atoms of the naphthalene ring. Therefore, the number of ring carbon atoms of the naphthalene ring substituted by the alkyl group is 10.

In this specification, the number of ring atoms represents the number of atoms forming a subject ring itself among the atoms of a compound having a structure in which atoms are bonded in a ring form (for example, the structure includes a monocyclic ring, a fused ring and a ring assembly) (for example, a monocyclic compound, a fused ring compound, a cross-linked compound, a carbocyclic compound and a heterocyclic compound). The number of ring atoms does not include atoms which do not form the ring (for example, a hydrogen atom which terminates a bond of the atoms forming the ring), or atoms contained in a substituent when the ring is substituted by the substituent. The same shall apply to “the number of ring atoms” described below, unless otherwise specified. For example, the number of atoms of a pyridine ring is 6, the number of atoms of a quinazoline ring is 101 and the number of a furan ring is 5. For example, hydrogen atoms bonded to a pyridine ring and atoms constituting a substituent substituted on the pyridine ring are not included in the number of ring atoms of the pyridine ring. Therefore, the number of ring atoms of a pyridine ring with which a hydrogen atom or a substituent is bonded is 6. For example, hydrogen atoms and atoms constituting a substituent which are bonded with a quinazoline ring is not included in the number of ring atoms of the quinazoline ring. Therefore, the number of ring atoms of a quinazoline ring with which a hydrogen atom or a substituent is bonded is 10.

In this specification, “XX to YY carbon atoms” in the expression “a substituted or unsubstituted ZZ group including XX to YY carbon atoms” represents the number of carbon atoms in the case where the ZZ group is unsubstituted by a substituent, and does not include the number of carbon atoms of a substituent in the case where the ZZ group is substituted by the substituent. Here, “YY” is larger than “XX”, and “XX” means an integer of 1 or more and “YY” means an integer of 2 or more.

In this specification, “XX to YY atoms” in the expression “a substituted or unsubstituted ZZ group including XX to YY atoms” represents the number of atoms in the case where the ZZ group is unsubstituted by a substituent, and does not include the number of atoms of a substituent in the case where the ZZ group is substituted by the substituent. Here, “YY” is larger than “XX”, and “XX” means an integer of 1 or more and “YY” means an integer of 2 or more.

In this specification, the unsubstituted ZZ group represents the case where the “substituted or unsubstituted ZZ group” is a “ZZ group unsubstituted by a substituent”, and the substituted ZZ group represents the case where the “substituted or unsubstituted ZZ group“is a” ZZ group substituted by a substituent”.

In this specification, a term “unsubstituted” in the case of “a substituted or unsubstituted ZZ group” means that hydrogen atoms in the ZZ group are not substituted by a substituent. Hydrogen atoms in a term “unsubstituted ZZ group” are a protium atom, a deuterium atom, or a tritium atom.

In this specification, a term “substituted” in the case of “a substituted or unsubstituted ZZ group” means that one or more hydrogen atoms in the ZZ group are substituted by a substituent. Similarly, a term “substituted” in the case of “a BB group substituted by an AA group” means that one or more hydrogen atoms in the BB group are substituted by the AA group.

“Substituent as Described in this Specification”

Hereinafter, the substituent described in this specification will be explained.

The number of ring carbon atoms of the “unsubstituted aryl group” described in this specification is 6 to 50, preferably 6 to 30, and more preferably 6 to 18, unless otherwise specified.

The number of ring atoms of the “unsubstituted heterocyclic group” described in this specification is 5 to 50, preferably 5 to 30, and more preferably 5 to 18, unless otherwise specified.

The number of carbon atoms of the “unsubstituted alkyl group” described in this specification is 1 to 50, preferably 1 to 20, and more preferably 1 to 6, unless otherwise specified.

The number of carbon atoms of the “unsubstituted alkenyl group” described in this specification is 2 to 50, preferably 2 to 20, and more preferably 2 to 6, unless otherwise specified.

The number of carbon atoms of the “unsubstituted alkynyl group” described in this specification is 2 to 50, preferably 2 to 20, and more preferably 2 to 6, unless otherwise specified.

The number of ring carbon atoms of the “unsubstituted cycloalkyl group” described in this specification is 3 to 50, preferably 3 to 20, and more preferably 3 to 6, unless otherwise specified.

The number of ring carbon atoms of the “unsubstituted arylene group” described in this specification is 6 to 50, preferably 6 to 30, and more preferably 6 to 18, unless otherwise specified.

The number of ring atoms of the “unsubstituted divalent heterocyclic group” described in this specification is 5 to 50, preferably 5 to 30, and more preferably 5 to 18, unless otherwise specified.

The number of carbon atoms of the “unsubstituted alkylene group” described in this specification is 1 to 50, preferably 1 to 20, and more preferably 1 to 6, unless otherwise specified.

### “Substituted or Unsubstituted Aryl Group”

Specific examples of the “substituted or unsubstituted aryl group” described in this specification (specific example group G1) include the following unsubstituted aryl groups (specific example group G1A), substituted aryl groups (specific example group G1B), and the like. (Here, the unsubstituted aryl group refers to the case where the “substituted or unsubstituted aryl group” is an “aryl group unsubstituted by a substituent”, and the substituted aryl group refers to the case where the “substituted or unsubstituted aryl group” is an “aryl group substituted by a substituent”). In this specification, in the case where simply referred as an “aryl group”, it includes both a “unsubstituted aryl group” and a “substituted aryl group.”

The “substituted aryl group” means a group in which one or more hydrogen atoms of the “unsubstituted aryl group” are substituted by a substituent. Specific examples of the “substituted aryl group” include, for example, groups in which one or more hydrogen atoms of the “unsubstituted aryl group” of the following specific example group G1A are substituted by a substituent, the substituted aryl groups of the following specific example group G1 B, and the like. It should be noted that the examples of the “unsubstituted aryl group” and the examples of the “substituted aryl group” enumerated in this specification are mere examples, and the “substituted aryl group” described in this specification also includes a group in which a hydrogen atom bonded with a carbon atom of the aryl group itself in the “substituted aryl group” of the following specific group G1 B is further substituted by a substituent, and a group in which a hydrogen atom of a substituent in the “substituted aryl group” of the following specific group G1 B is further substituted by a substituent.

### Unsubstituted Aryl Group (Specific Example Group G1A):

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenalenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
a monovalent aryl group derived by removing one hydrogen atom from the ring structures represented by any of the following general formulas (TEMP-1) to (TEMP-15).

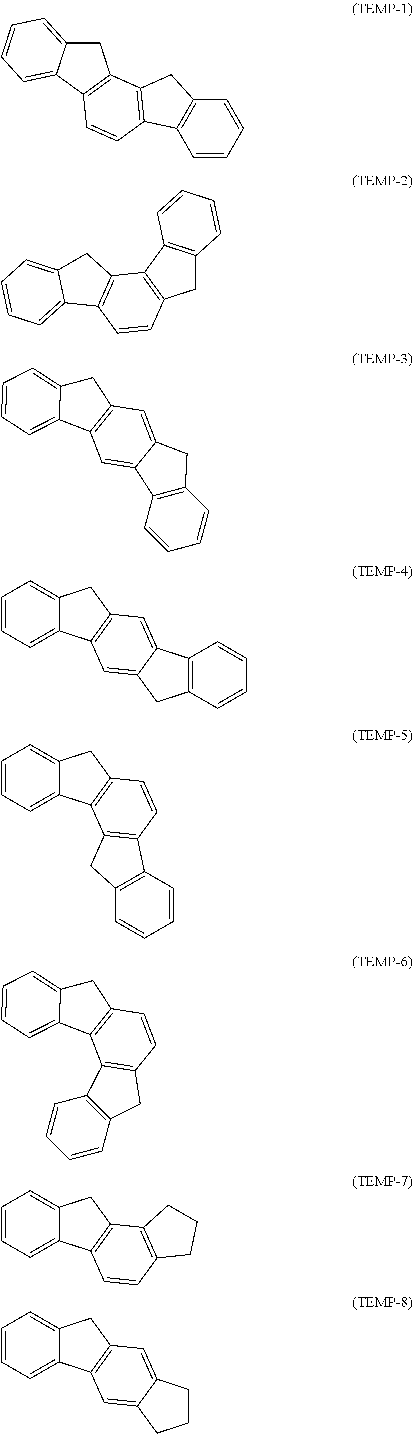

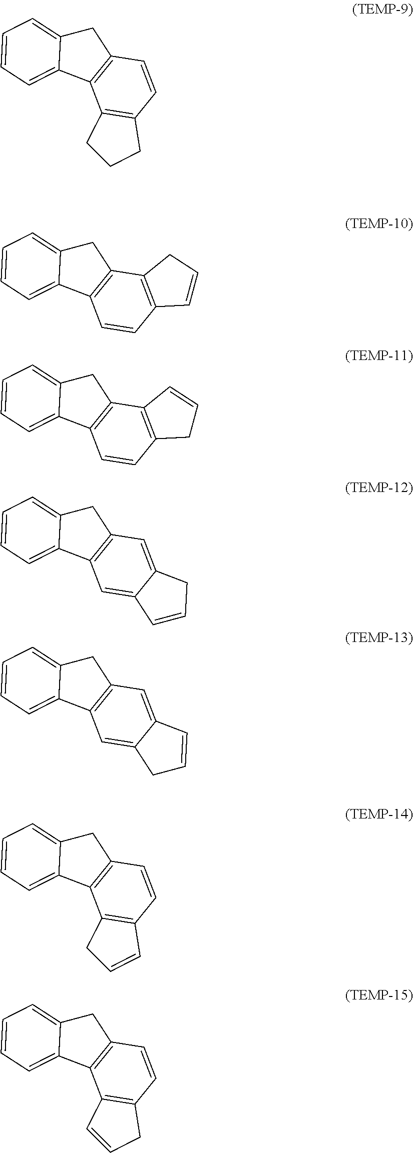

Substituted aryl group (specific example group G1 B):
an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
a group in which one or more hydrogen atoms of a monovalent group derived from the ring structures represented by any of the general formulas (TEMP-1) to (TEMP-15) are substituted by a substituent.

### “Substituted or Unsubstituted Heterocyclic Group”

The “heterocyclic group” described in this specification is a ring group having at least one hetero atom in the ring atom. Specific examples of the hetero atom include a nitrogen atom, an oxygen atom, a sulfur atom, a silicon atom, a phosphorus atom, and a boron atom.

The “heterocyclic group” in this specification is a monocyclic group or a fused ring group.

The “heterocyclic group” in this specification is an aromatic heterocyclic group or a non-aromatic heterocyclic group.

Specific examples of the “substituted or unsubstituted heterocyclic group” (specific example group G2) described in this specification include the following unsubstituted heterocyclic group (specific example group G2A), the following substituted heterocyclic group (specific example group G2B), and the like. (Here, the unsubstituted heterocyclic group refers to the case where the “substituted or unsubstituted heterocyclic group” is a “heterocyclic group unsubstituted by a substituent”, and the substituted heterocyclic group refers to the case where the “substituted or unsubstituted heterocyclic group“is a” heterocyclic group substituted by a substituent”). In this specification, in the case where simply referred as a “heterocyclic group”, it includes both the “unsubstituted heterocyclic group” and the “substituted heterocyclic group.”

The “substituted heterocyclic group” means a group in which one or more hydrogen atom of the “unsubstituted heterocyclic group” are substituted by a substituent. Specific examples of the “substituted heterocyclic group” include a group in which a hydrogen atom of “unsubstituted heterocyclic group” of the following specific example group G2A is substituted by a substituent, the substituted heterocyclic groups of the following specific example group G2B, and the like. It should be noted that the examples of the “unsubstituted heterocyclic group” and the examples of the “substituted heterocyclic group” enumerated in this specification are mere examples, and the “substituted heterocyclic group” described in this specification includes groups in which hydrogen atom bonded with a ring atom of the heterocyclic group itself in the “substituted heterocyclic group” of the specific example group G2B is further substituted by a substituent, and a group in which hydrogen atom of a substituent in the “substituted heterocyclic group” of the specific example group G2B is further substituted by a substituent.

Specific example group G2A includes, for example, the following unsubstituted heterocyclic group containing a nitrogen atom (specific example group G2A1), the following unsubstituted heterocyclic group containing an oxygen atom (specific example group G2A2), the following unsubstituted heterocyclic group containing a sulfur atom (specific example group G2A3), and the monovalent heterocyclic group derived by removing one hydrogen atom from the ring structures represented by any of the following general formulas (TEMP-16) to (TEMP-33) (specific example group G2A4).

Specific example group G2B includes, for example, the following substituted heterocyclic group containing a nitrogen atom (specific example group G2B1), the following substituted heterocyclic group containing an oxygen atom (specific example group G2B2), the following substituted heterocyclic group containing a sulfur atom (specific example group G2B3), and the following group in which one or more hydrogen atoms of the monovalent heterocyclic group derived from the ring structures represented by any of the following general formulas (TEMP-16) to (TEMP-33) are substituted by a substituent (specific example group G2B4).

### Unsubstituted Heterocyclic Group Containing a Nitrogen Atom (Specific Example Group G2A1):

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

### Unsubstituted Heterocyclic Group Containing an Oxygen Atom (Specific Example Group G2A2):

a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

### Unsubstituted Heterocyclic Group Containing a Sulfur Atom (Specific Example Group G2A3):

a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).
   Monovalent heterocyclic group derived by removing one hydrogen atom from the ring structures represented by any of the following general formulas (TEMP-16) to (TEMP-33) (specific example group G2A4):

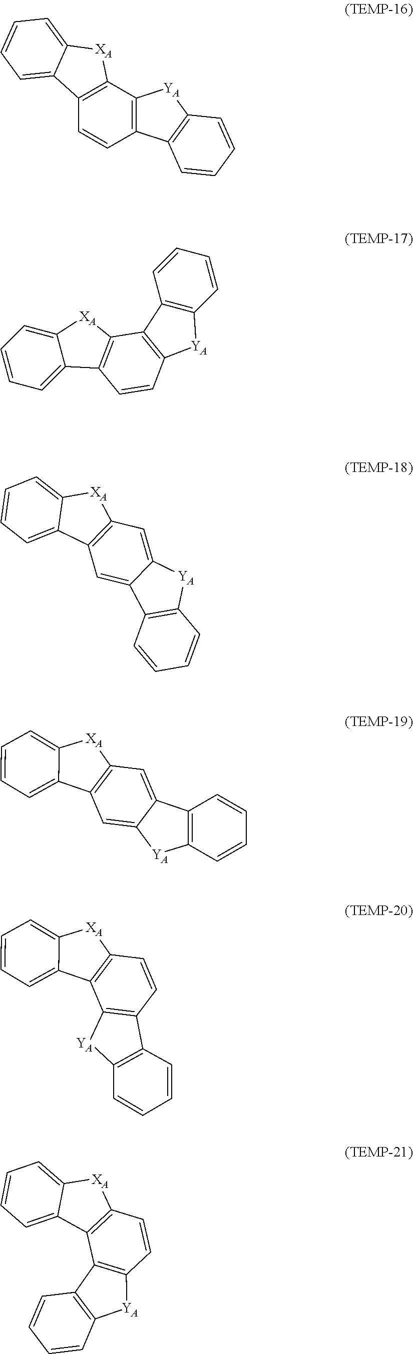

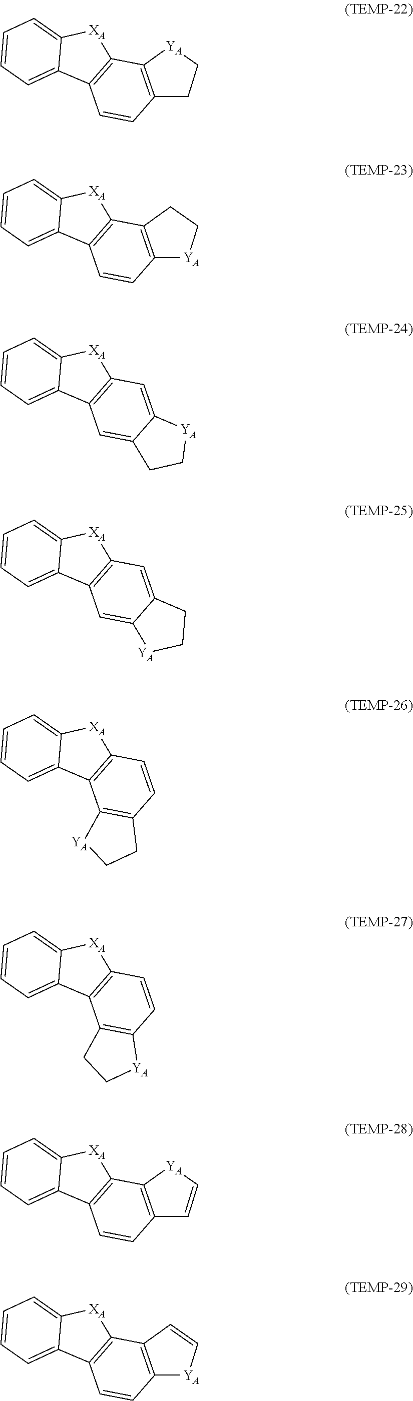

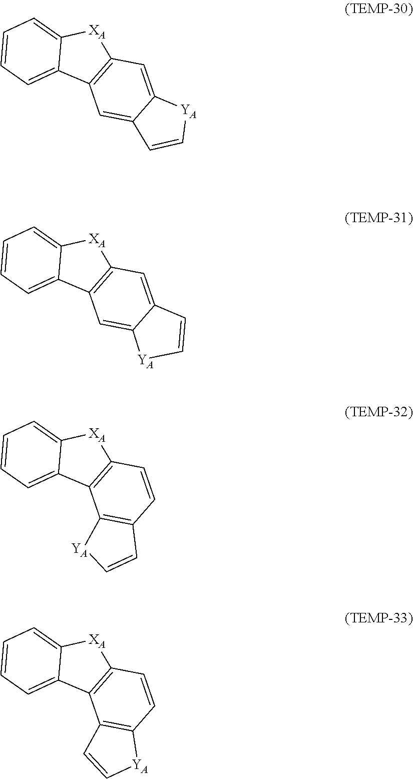

In the general formulas (TEMP-16) to (TEMP-33), XA and YA are independently an oxygen atom, a sulfur atom, NH, or CH₂. Provided that at least one of XA and YA is an oxygen atom, a sulfur atom, or NH.

In the general formulas (TEMP-16) to (TEMP-33), when at least one of XA and YA is NH or CH₂, the monovalent heterocyclic group derived from the ring structures represented by any of the general formulas (TEMP-16) to (TEMP-33) includes a monovalent group derived by removing one hydrogen atom from these NH or CH₂.

Substituted heterocyclic group containing a nitrogen atom (specific example group G2B1):
a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenylyltriazinyl group,
a diphenyltriazinyl group,
a phenylquinazolinyl group, and
a biphenylylquinazolinyl group.

### Substituted Heterocyclic Group Containing an Oxygen Atom (Specific Example Group G2B2):

a phenyldibenzofuranyl group,
a methyldibenzofuranyl group,
a t-butyldibenzofuranyl group, and
a monovalent residue of spiro[9H-xanthene-9,9′-[9H]fluorene].

### Substituted Heterocyclic Group Containing a Sulfur Atom (Specific Example Group G2B3):

a phenyldibenzothiophenyl group,
a methyldibenzothiophenyl group,
a t-butyldibenzothiophenyl group, and
a monovalent residue of spiro[9H-thioxanthene-9,9′-[9H]fluorene].
   Group in which one or more hydrogen atoms of the monovalent heterocyclic group derived from the ring structures represented by any of the following general formulas (TEMP-16) to (TEMP-33) are substituted by a substituent (specific example group G2B4):

The “one or more hydrogen atoms of the monovalent heterocyclic group” means one or more hydrogen atoms selected from hydrogen atoms bonded with ring carbon atoms of the monovalent heterocyclic group, a hydrogen atom bonded with a nitrogen atom when at least one of XA and YA is NH, and hydrogen atoms of a methylene group when one of XA and YA is CH₂.

### “Substituted or Unsubstituted Alkyl Group”

Specific examples of the “substituted or unsubstituted alkyl group” (specific example group G3) described in this specification include the following unsubstituted alkyl groups (specific example group G3A) and the following substituted alkyl groups (specific example group G3B). (Here, the unsubstituted alkyl group refers to the case where the “substituted or unsubstituted alkyl group” is an “alkyl group unsubstituted by a substituent”, and the substituted alkyl group refers to the case where the “substituted or unsubstituted alkyl group” is an “alkyl group substituted by a substituent”). In this specification, in the case where simply referred as an “alkyl group” includes both the “unsubstituted alkyl group” and the “substituted alkyl group.”

The “substituted alkyl group” means a group in which one or more hydrogen atoms in the “unsubstituted alkyl group” are substituted by a substituent. Specific examples of the “substituted alkyl group” include groups in which one or more hydrogen atoms in the following “unsubstituted alkyl group” (specific example group G3A) are substituted by a substituent, the following substituted alkyl group (specific example group G3B), and the like. In this specification, the alkyl group in the “unsubstituted alkyl group” means a linear alkyl group. Thus, the “unsubstituted alkyl group” includes a straight-chain “unsubstituted alkyl group” and a branched-chain “unsubstituted alkyl group”. It should be noted that the examples of the “unsubstituted alkyl group” and the examples of the “substituted alkyl group” enumerated in this specification are mere examples, and the “substituted alkyl group” described in this specification includes a group in which hydrogen atom of the alkyl group itself in the “substituted alkyl group” of the specific example group G3B is further substituted by a substituent, and a group in which hydrogen atom of a substituent in the “substituted alkyl group” of the specific example group G3B is further substituted by a substituent.

### Unsubstituted Alkyl Group (Specific Example Group G3A):

a methyl group,
an ethyl group,
a n-propyl group,
an isopropyl group,
a n-butyl group,
an isobutyl group,
a s-butyl group, and
a t-butyl group.

### Substituted Alkyl Group (Specific Example Group G3B):

a heptafluoropropyl group (including isomers),
a pentafluoroethyl group,
a 2,2,2-trifluoroethyl group, and
a trifluoromethyl group.

### “Substituted or Unsubstituted Alkenyl Group”

Specific examples of the “substituted or unsubstituted alkenyl group” described in this specification (specific example group G4) include the following unsubstituted alkenyl group (specific example group G4A), the following substituted alkenyl group (specific example group G4B), and the like. (Here, the unsubstituted alkenyl group refers to the case where the “substituted or unsubstituted alkenyl group“is a” alkenyl group unsubstituted by a substituent”, and the “substituted alkenyl group” refers to the case where the “substituted or unsubstituted alkenyl group” is a “alkenyl group substituted by a substituent.”). In this specification, in the case where simply referred as an “alkenyl group” includes both the “unsubstituted alkenyl group” and the “substituted alkenyl group.”

The “substituted alkenyl group” means a group in which one or more hydrogen atoms in the “unsubstituted alkenyl group” are substituted by a substituent. Specific examples of the “substituted alkenyl group” include a group in which the following “unsubstituted alkenyl group” (specific example group G4A) has a substituent, the following substituted alkenyl group (specific example group G4B), and the like. It should be noted that the examples of the “unsubstituted alkenyl group” and the examples of the “substituted alkenyl group” enumerated in this specification are mere examples, and the “substituted alkenyl group” described in this specification includes a group in which a hydrogen atom of the alkenyl group itself in the “substituted alkenyl group” of the specific example group G4B is further substituted by a substituent, and a group in which a hydrogen atom of a substituent in the “substituted alkenyl group” of the specific example group G4B is further substituted by a substituent.

### Unsubstituted Alkenyl Group (Specific Example Group G4A):

a vinyl group,
an allyl group,
a 1-butenyl group,
a 2-butenyl group, and
a 3-butenyl group.

### Substituted Alkenyl Group (Specific Example Group G4B):

a 1,3-butanedienyl group,
a 1-methylvinyl group,
a 1-methylallyl group,
a 1,1-dimethylallyl group,
a 2-methylally group, and
a 1,2-dimethylallyl group.

### “Substituted or Unsubstituted Alkynyl Group”

Specific examples of the “substituted or unsubstituted alkynyl group” described in this specification (specific example group G5) include the following unsubstituted alkynyl group (specific example group G5A) and the like. (Here, the unsubstituted alkynyl group refers to the case where the “substituted or unsubstituted alkynyl group” is an “alkynyl group unsubstituted by a substituent”). In this specification, in the case where simply referred as an “alkynyl group” includes both the “unsubstituted alkynyl group” and the “substituted alkynyl group.”

The “substituted alkynyl group” means a group in which one or more hydrogen atoms in the “unsubstituted alkynyl group” are substituted by a substituent. Specific examples of the “substituted alkynyl group” include a group in which one or more hydrogen atoms in the following “unsubstituted alkynyl group” (specific example group G5A) are substituted by a substituent, and the like.

### Unsubstituted Alkynyl Group (Specific Example Group G5A):

an ethynyl group.

### “Substituted or Unsubstituted Cycloalkyl Group”

Specific examples of the “substituted or unsubstituted cycloalkyl group” described in this specification (specific example group G6) include the following unsubstituted cycloalkyl group (specific example group G6A), the following substituted cycloalkyl group (specific example group G6B), and the like. (Here, the unsubstituted cycloalkyl group refers to the case where the “substituted or unsubstituted cycloalkyl group” is a “cycloalkyl group unsubstituted by a substituent”, and the substituted cycloalkyl group refers to the case where the “substituted or unsubstituted cycloalkyl group“is a” cycloalkyl group substituted by a substituent”). In this specification, in the case where simply referred as a “cycloalkyl group” includes both the “unsubstituted cycloalkyl group” and the “substituted cycloalkyl group.”

The “substituted cycloalkyl group” means a group in which one or more hydrogen atoms in the “unsubstituted cycloalkyl group” are substituted by a substituent. Specific examples of the “substituted cycloalkyl group” include a group in which one or more hydrogen atoms in the following “unsubstituted cycloalkyl group” (specific example group G6A) are substituted by a substituent, and examples of the following substituted cycloalkyl group (specific example group G6B), and the like. It should be noted that the examples of the “unsubstituted cycloalkyl group” and the examples of the “substituted cycloalkyl group” enumerated in this specification are mere examples, and the “substituted cycloalkyl group” in this specification includes a group in which one or more hydrogen atoms bonded with the carbon atom of the cycloalkyl group itself in the “substituted cycloalkyl group” of the specific example group G6B are substituted by a substituent, and a group in which a hydrogen atom of a substituent in the “substituted cycloalkyl group” of specific example group G6B is further substituted by a substituent.

### Unsubstituted Cycloalkyl Group (Specific Example Group G6A):

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

### Substituted Cycloalkyl Group (Specific Example Group G6B):

a 4-methylcyclohexyl group.
   “Group Represented by —Si(R₉₀₁)(R₉₀₂)(R₉₀₃)”

Specific examples of the group represented by —Si(R₉₀₁)(R₉₀₂)(R₉₀₃) described in this specification (specific example group G7) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.
Plural G1's in —Si(G1)(G1)(G1) are the same or different.
Plural G2's in —Si(G1)(G2)(G2) are the same or different.
Plural G1's in —Si(G1)(G1)(G2) are the same or different.
Plural G2's in —Si(G2)(G2)(G2) are be the same or different.
Plural G3's in —Si(G3)(G3)(G3) are the same or different.
Plural G6's in —Si(G6)(G6)(G6) are be the same or different.

### “Group Represented by —O—(R₉₀₄)”

Specific examples of the group represented by —O—(R₉₀₄) in this specification (specific example group G8) include:
—O(G1),
—O(G2),
—O(G3), and
—O(G6).
G1 is the “substituted or unsubstituted aryl group” described in the specific example group
G1.
G2 is the “substituted or unsubstituted heterocyclic group” described in the specific
example group G2.
G3 is the “substituted or unsubstituted alkyl group” described in the specific example group
G3.
G6 is the “substituted or unsubstituted cycloalkyl group” described in the specific example
group G6.

### “Group Represented by —S—(R₉₀₅)”

Specific examples of the group represented by —S—(R₉₀₅) in this specification (specific example group G9) include:
—S(G1),
—S(G2),
—S(G3), and
—S(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.
   “Group Represented by —N(R₉₀₆)(R₉₀₇)”

Specific examples of the group represented by —N(R₉₀₅)(R₉₀₇) in this specification (specific example group G10) include:
—N(G1)(G1),
—N(G2)(G2),
—N(G1)(G2),
—N(G3)(G3), and
—N(G6)(G6).
G1 is the “substituted or unsubstituted aryl group” described in the specific example group
G1.
G2 is the “substituted or unsubstituted heterocyclic group” described in the specific
example group G2.
G3 is the “substituted or unsubstituted alkyl group” described in the specific example group
G3.
G6 is the “substituted or unsubstituted cycloalkyl group” described in the specific example
group G6.
Plural G1's in —N(G1)(G1) are the same or different.
Plural G2's in —N(G2)(G2) are the same or different.
Plural G3's in —N(G3)(G3) are the same or different.
Plural G6's in —N(G6)(G6) are the same or different.

### “Halogen Atom”

Specific examples of the “halogen atom” described in this specification (specific example group G11) include a fluorine atom, a chlorine atom, a bromine atom, an iodine atom, and the like.

### “Substituted or Unsubstituted Fluoroalkyl Group”

The “substituted or unsubstituted fluoroalkyl group” described in this specification is a group in which at least one hydrogen atom bonded with a carbon atom constituting the alkyl group in the “substituted or unsubstituted alkyl group” is substituted by a fluorine atom, and includes a group in which all hydrogen atoms bonded with a carbon atom constituting the alkyl group in the “substituted or unsubstituted alkyl group” are substituted by a fluorine atom (a perfluoro group). The number of carbon atoms of the “unsubstituted fluoroalkyl group” is 1 to 50, preferably 1 to 30, more preferably 1 to 18, unless otherwise specified in this specification. The “substituted fluoroalkyl group” means a group in which one or more hydrogen atoms of the “fluoroalkyl group” are substituted by a substituent. The “substituted fluoroalkyl group” described in this specification also includes a group in which one or more hydrogen atoms bonded with a carbon atom of the alkyl chains in the “substituted fluoroalkyl group” are further substituted by a substituent, and a group in which one or more hydrogen atom of a substituent in the “substituted fluoroalkyl group” are further substituted by a substituent. Specific examples of the “unsubstituted fluoroalkyl group” include a group in which one or more hydrogen atoms in the “alkyl group” (specific group G3) are substituted by a fluorine atom, and the like.

### “Substituted or Unsubstituted Haloalkyl Group”

The “substituted or unsubstituted haloalkyl group” described in this specification is a group in which at least one hydrogen atom bonded with a carbon atom constituting the alkyl group in the “substituted or unsubstituted alkyl group” is substituted by a halogen atom, and also includes a group in which all hydrogen atoms bonded with a carbon atom constituting the alkyl group in the “substituted or unsubstituted alkyl group” are substituted by a halogen atom. The number of carbon atoms of the “unsubstituted haloalkyl group” is 1 to 50, preferably 1 to 30, more preferably 1 to 18, unless otherwise specified in this specification. The “substituted haloalkyl group” means a group in which one or more hydrogen atoms of the “haloalkyl group” are substituted by a substituent. The “substituted haloalkyl group” described in this specification also includes a group in which one or more hydrogen atoms bonded with a carbon atom of the alkyl chain in the “substituted haloalkyl group” are further substituted by a substituent, and a group in which one or more hydrogen atoms of a substituent in the “substituted haloalkyl group” are further substituted by a substituent. Specific examples of the “unsubstituted haloalkyl group” include a group in which one or more hydrogen atoms in the “alkyl group” (specific example group G3) are substituted by a halogen atom, and the like. A haloalkyl group is sometimes referred to as an alkyl halide group.

### “Substituted or Unsubstituted Alkoxy Group”

Specific examples of the “substituted or unsubstituted alkoxy group” described in this specification include a group represented by —O(G3), wherein G3 is the “substituted or unsubstituted alkyl group” described in the specific example group G3. The number of carbon atoms of the “unsubstituted alkoxy group” is 1 to 50, preferably 1 to 30, more preferably 1 to 18, unless otherwise specified in this specification.

### “Substituted or Unsubstituted Alkylthio Group”

Specific examples of the “substituted or unsubstituted alkylthio group” described in this specification include a group represented by —S(G3), wherein G3 is the “substituted or unsubstituted alkyl group” described in the specific example group G3. The number of carbon atoms of the “unsubstituted alkylthio group” is 1 to 50, preferably 1 to 30, more preferably 1 to 18, unless otherwise specified in this specification.

### “Substituted or Unsubstituted Aryloxy Group”

Specific examples of the “substituted or unsubstituted aryloxy group” described in this specification include a group represented by —O(G1), wherein G1 is the “substituted or unsubstituted aryl group” described in the specific example group G1. The number of ring carbon atoms of the “unsubstituted aryloxy group” is 6 to 50, preferably 6 to 30, more preferably 6 to 18, unless otherwise specified in this specification.

### “Substituted or Unsubstituted Arylthio Group”

Specific examples of the “substituted or unsubstituted arylthio group” described in this specification include a group represented by —S(G1), wherein G1 is a “substituted or unsubstituted aryl group” described in the specific example group G1. The number of ring carbon atoms of the “unsubstituted arylthio group” is 6 to 50, preferably 6 to 30, more preferably 6 to 18, unless otherwise specified in this specification.

### “Substituted or Unsubstituted Trialkylsilyl Group”

Specific examples of the “trialkylsilyl group” described in this specification include a group represented by —Si(G3)(G3)(G3), where G3 is the “substituted or unsubstituted alkyl group” described in the specific example group G3. Plural G3's in —Si(G3)(G3)(G3) are the same or different. The number of carbon atoms in each alkyl group of the “trialkylsilyl group” is 1 to 50, preferably 1 to 20, more preferably 1 to 6, unless otherwise specified in this specification.

### “Substituted or Unsubstituted Aralkyl Group”

Specific examples of the “substituted or unsubstituted aralkyl group” described in this specification is a group represented by -(G3)-(G1), wherein G3 is the “substituted or unsubstituted alkyl group” described in the specific example group G3, and G1 is the “substituted or unsubstituted aryl group” described in the specific example group G1. Therefore, the “aralkyl group” is a group in which a hydrogen atom of the “alkyl group” is substituted by an “aryl group” as a substituent, and is one form of the “substituted alkyl group.” The “unsubstituted aralkyl group” is the “unsubstituted alkyl group” substituted by the “unsubstituted aryl group”, and the number of carbon atoms of the “unsubstituted aralkyl group” is 7 to 50, preferably 7 to 30, more preferably 7 to 18, unless otherwise specified in this specification.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an a-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, and the like.

Unless otherwise specified in this specification, examples of the substituted or unsubstituted aryl group described in this specification preferably include a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, 9,9-dimethylfluorenyl group, 9,9-diphenylfluorenyl group, and the like.

Unless otherwise specified in this specification, examples of the substituted or unsubstituted heterocyclic groups described in this specification preferably include a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like.

In this specification, the carbazolyl group is specifically any of the following groups, unless otherwise specified in this specification.

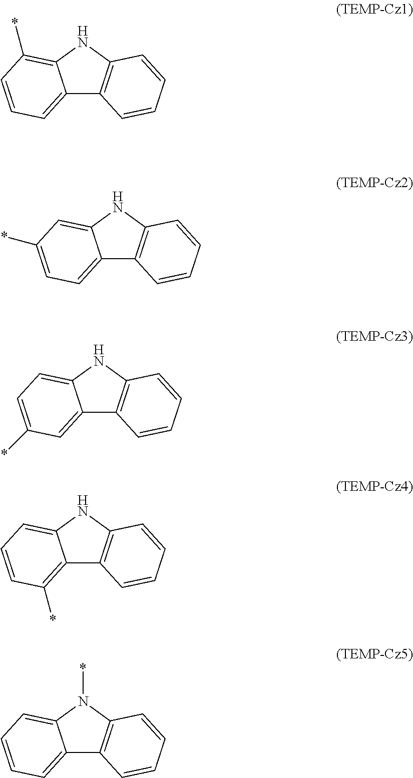 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In this Specification, the (9-Phenyl)Carbazolyl Group is Specifically any of the Following Groups, Unless Otherwise Specified in this Specification.

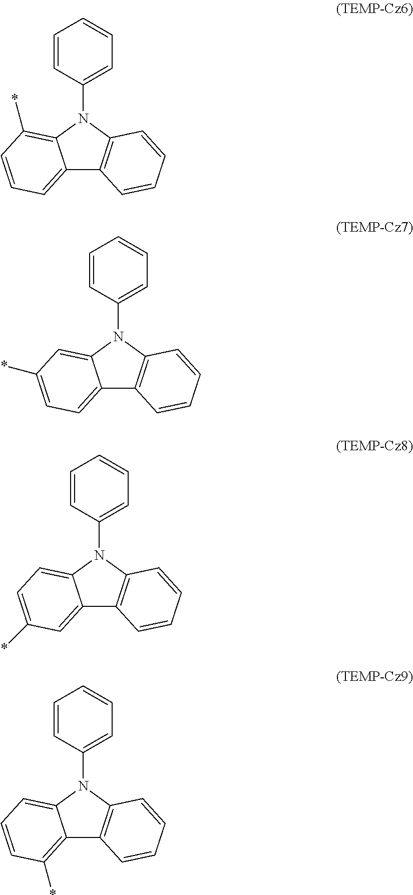

In the general formulas (TEMP-Cz1) to (TEMP-Cz9), * represents a bonding site.

In this specification, the dibenzofuranyl group and the dibenzothiophenyl group are specifically any of the following groups, unless otherwise specified in this specification.

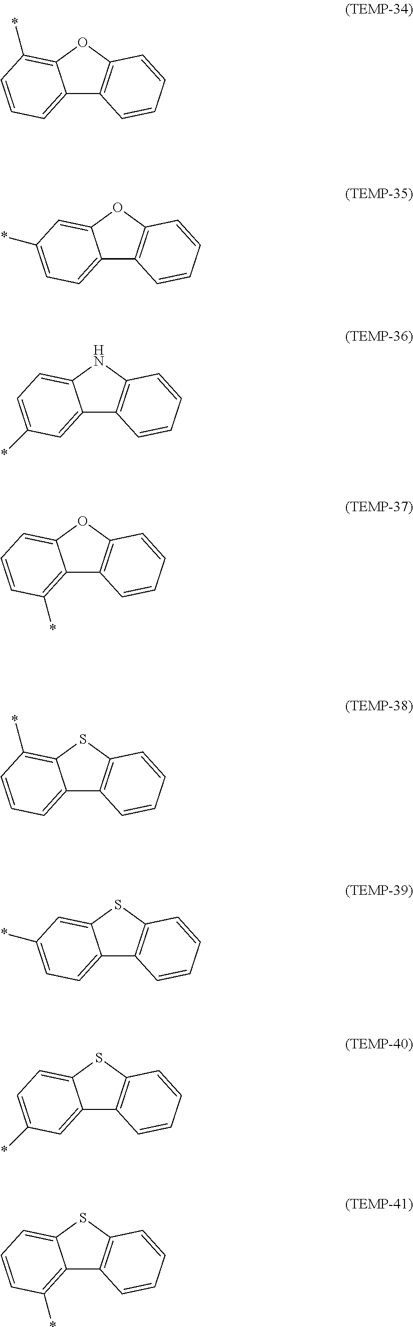

In the general formulas (TEMP-34) to (TEMP-41), * represents a bonding site.

The substituted or unsubstituted alkyl group described in this specification is preferably a methyl group, an ethyl group, a propyl group, an isopropyl group, a n-butyl group, an isobutyl group, a t-butyl group, or the like, unless otherwise specified in this specification.

### “Substituted or Unsubstituted Arylene Group”

The “substituted or unsubstituted arylene group” described in this specification is a divalent group derived by removing one hydrogen atom on the aryl ring of the “substituted or unsubstituted aryl group”, unless otherwise specified. Specific examples of the “substituted or unsubstituted arylene group” (specific example group G12) include a divalent group derived by removing one hydrogen atom on the aryl ring of the “substituted or unsubstituted aryl group” described in the specific example group G1, and the like.

### “Substituted or Unsubstituted Divalent Heterocyclic Group”

The “substituted or unsubstituted divalent heterocyclic group” described in this specification is a divalent group derived by removing one hydrogen atom on the heterocycle of the “substituted or unsubstituted heterocyclic group”, unless otherwise specified. Specific examples of the “substituted or unsubstituted divalent heterocyclic group” (specific example group G13) include a divalent group derived by removing one hydrogen atom on the heterocycle of the “substituted or unsubstituted heterocyclic group” described in the specific example group G2, and the like.

### “Substituted or Unsubstituted Alkylene Group”

The “substituted or unsubstituted alkylene group” described in this specification is a divalent group derived by removing one hydrogen atom on the alkyl chain of the “substituted or unsubstituted alkyl group”, unless otherwise specified. Specific examples of the “substituted or unsubstituted alkylene group” (specific example group G14) include a divalent group derived by removing one hydrogen atom on the alkyl chain of the “substituted or unsubstituted alkyl group” described in the specific example group G3, and the like.

The substituted or unsubstituted arylene group described in this specification is preferably any group of the following general formulas (TEMP-42) to (TEMP-68), unless otherwise specified in this specification.

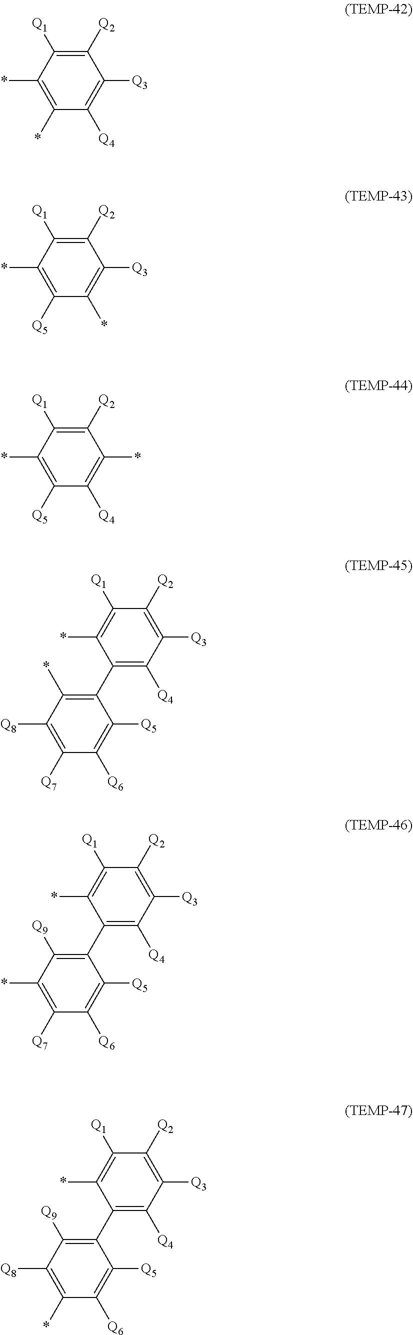

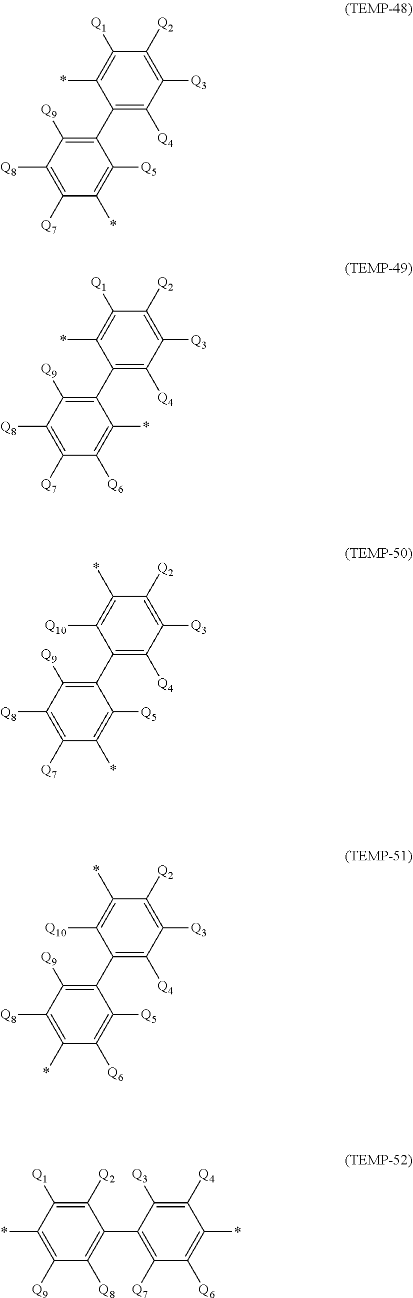

In the general formulas (TEMP-42) to (TEMP-52), Q₁ to Q₁₀ are independently a hydrogen atom or a substituent.

In the general formulas (TEMP-42) to (TEMP-52), * represents a bonding site.

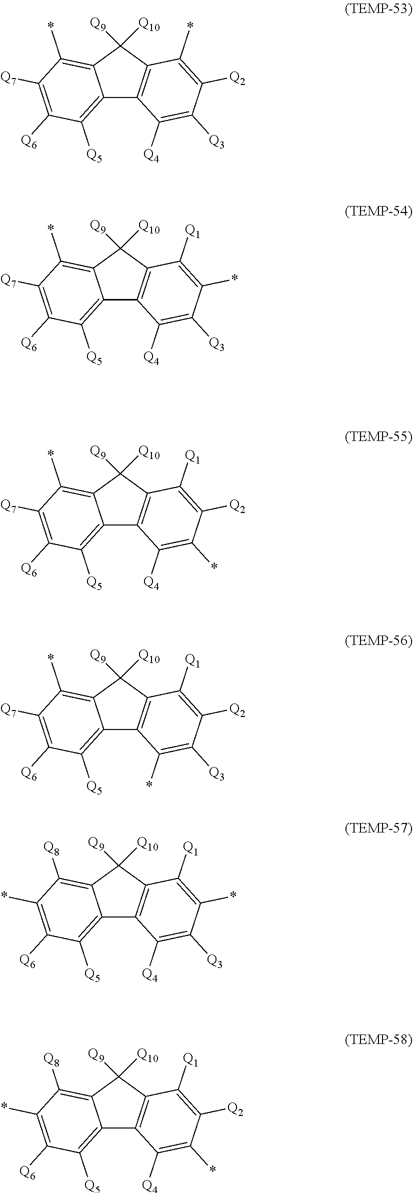

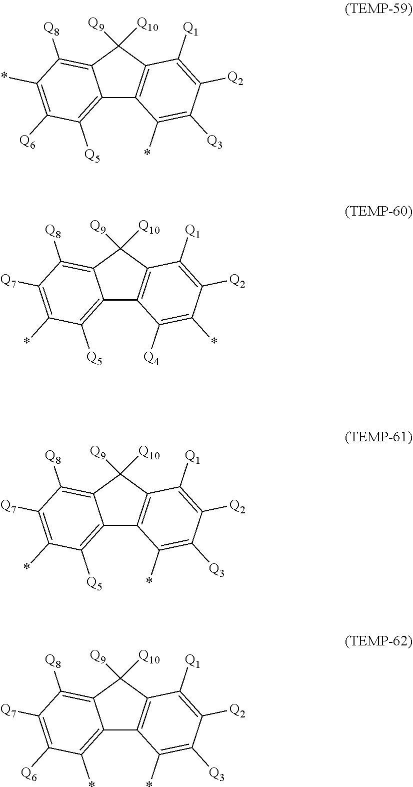

In the general formulas (TEMP-53) to (TEMP-62), Q₁ to Q₁₀ are independently a hydrogen atom or a substituent.

Q₉ and Q₁₀ may be bonded with each other via a single bond to form a ring.

In the general formulas (TEMP-53) to (TEMP-62), * represents a bonding site.

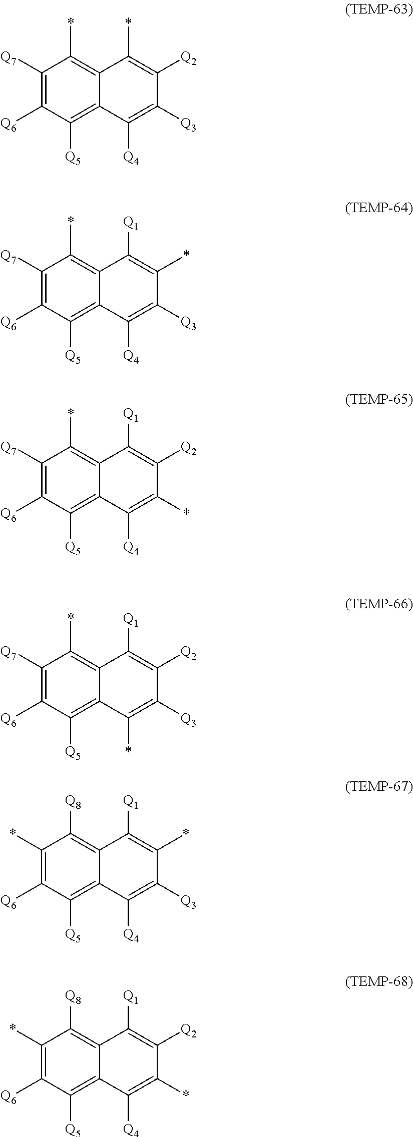

In the general formulas (TEMP-63) to (TEMP-68), Q₁ to Q₈ are independently a hydrogen atom or a substituent.

In the general formulas (TEMP-63) to (TEMP-68), * represents a bonding site.

The substituted or unsubstituted divalent heterocyclic group described in this specification is preferably any group of the following general formulas (TEMP-69) to (TEMP-102), unless otherwise specified in this specification.

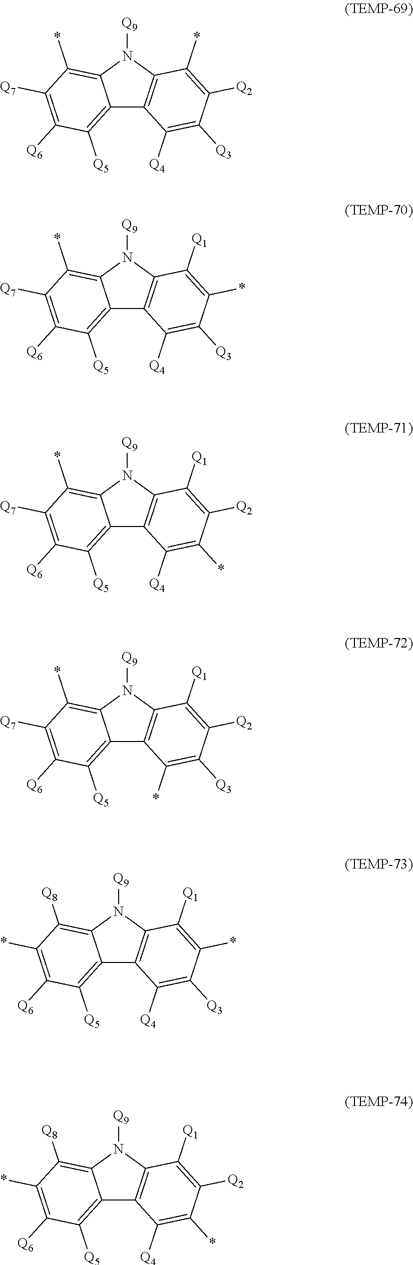

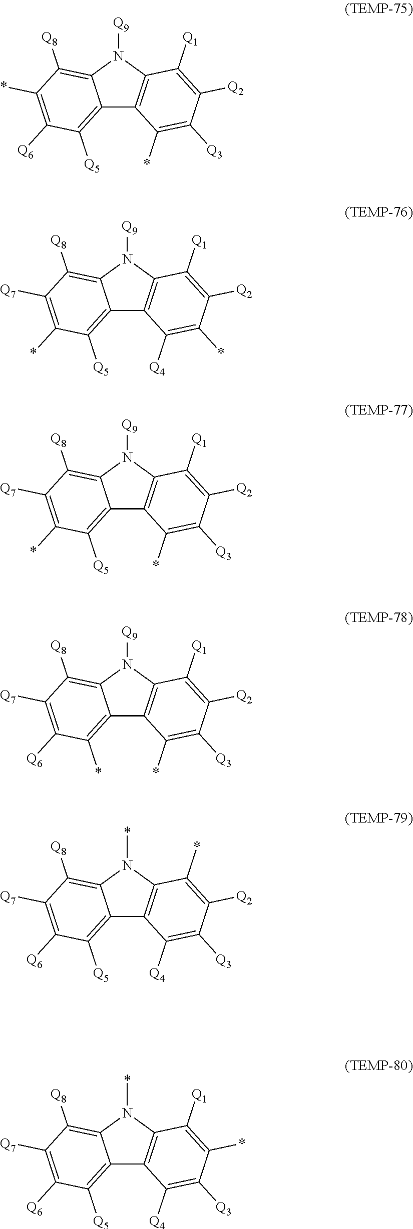

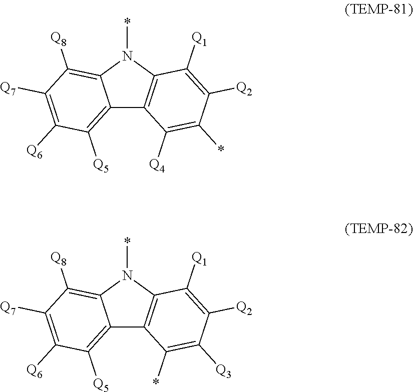

In the general formulas (TEMP-69) to (TEMP-82), Q₁ to Q₉ are independently a hydrogen atom or a substituent.

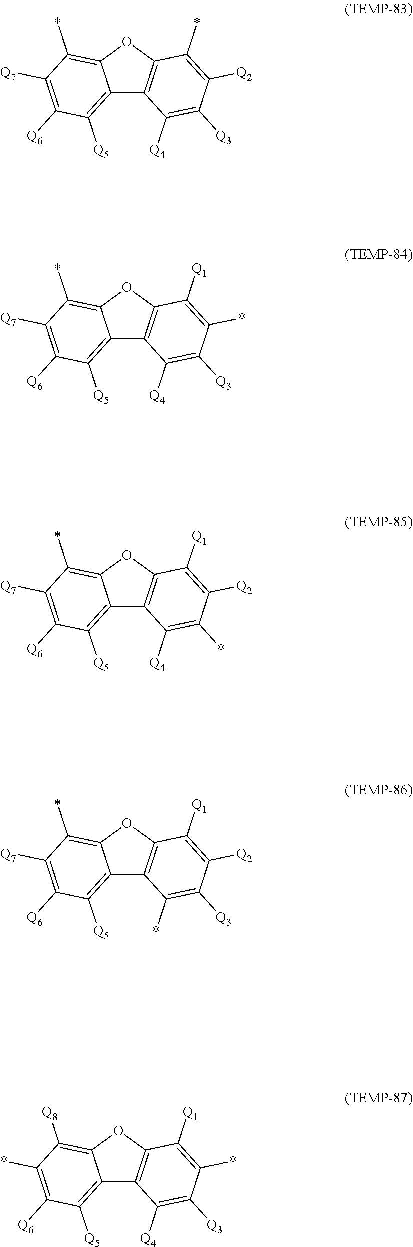

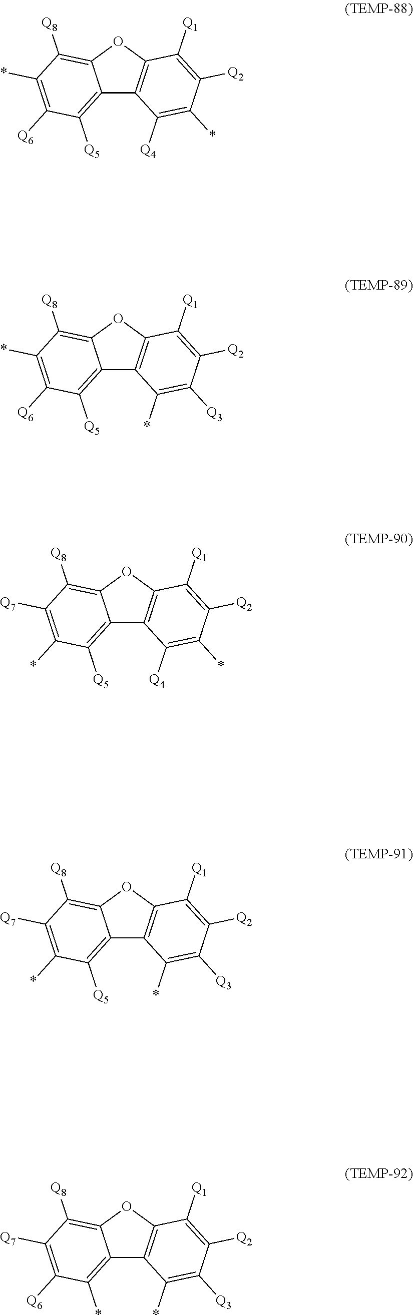

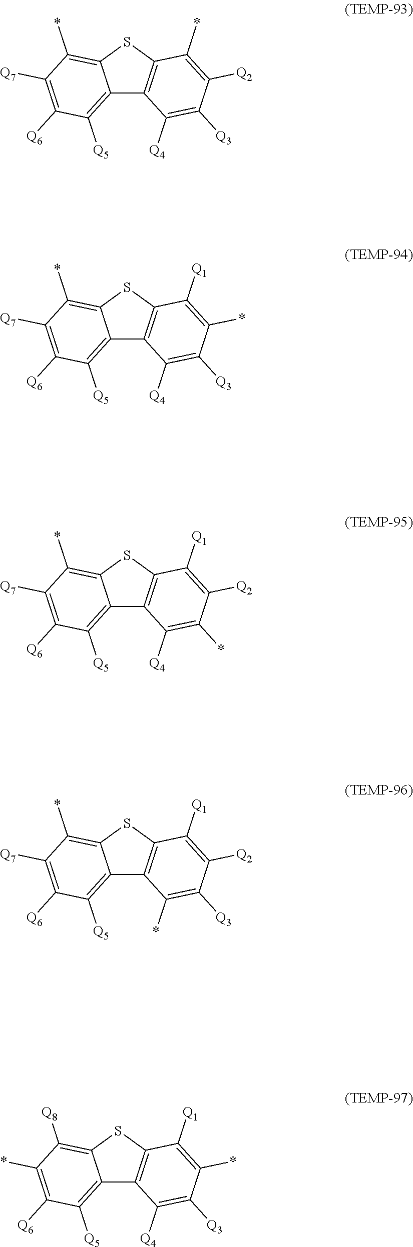

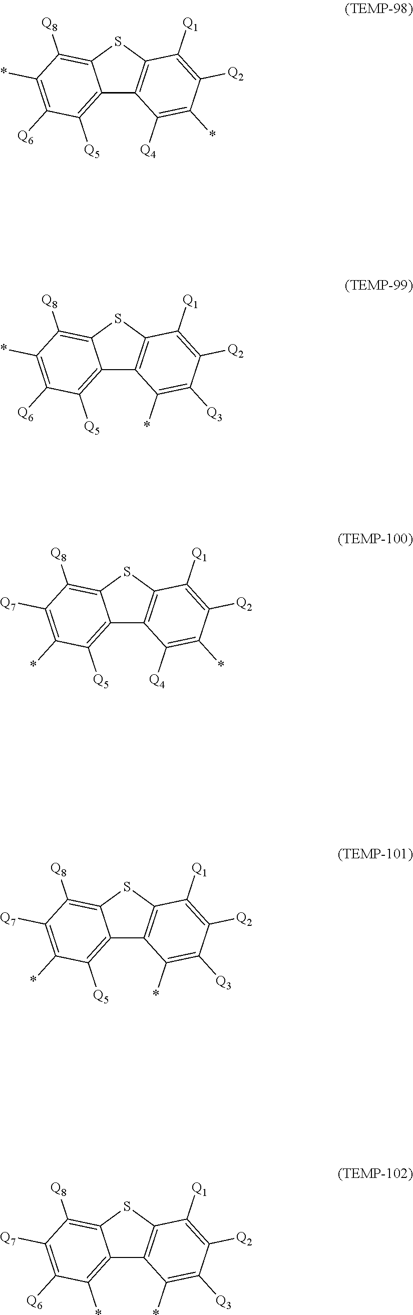

In the general formulas (TEMP-83) to (TEMP-102), Q₁ to Q₈ are independently a hydrogen atom or a substituent.

The above is the explanation of the “Substituent described in this specification.”

“The Case where Bonded with Each Other to Form a Ring”

In this specification, the case where “one or more sets of adjacent two or more form a substituted or unsubstituted monocycle by bonding with each other, form a substituted or unsubstituted fused ring by bonding with each other, or do not bond with each other” means the case where “one or more sets of adjacent two or more form a substituted or unsubstituted monocycle by bonding with each other”; the case where “one or more sets of adjacent two or more form a substituted or unsubstituted fused ring by bonding with each other”; and the case where “one or more sets of adjacent two or more do not bond with each other.”

The case where “one or more sets of adjacent two or more form a substituted or unsubstituted monocycle by bonding with each other” and the case where “one or more sets of adjacent two or more form a substituted or unsubstituted fused ring by bonding with each other” in this specification (these cases may be collectively referred to as “the case where forming a ring by bonding with each other”) will be described below. The case of an anthracene compound represented by the following general formula (TEMP-103) in which the mother skeleton is an anthracene ring will be described as an example.

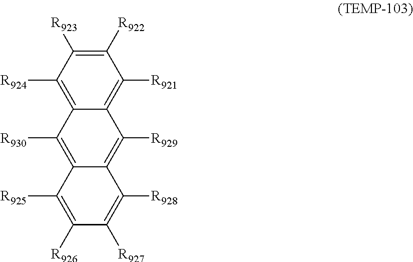

For example, in the case where “one or more sets of adjacent two or more among R₉₂₁ to R₉₃₀ form a ring by bonding with each other”, the one set of adjacent two includes a pair of R₉₂₁ and R₉₂₂, a pair of R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₈, a pair of R₉₂₈ and R₉₂₉, and a pair of R₉₂₉ and R₉₂₁.

The “one or more sets” means that two or more sets of the adjacent two or more sets may form a ring at the same time. For example, R₉₂₁ and R₉₂₂ form a ring Q_{A} by bonding with each other, and at the same, time R₉₂₅ and R₉₂₆ form a ring Q_{B} by bonding with each other, the anthracene compound represented by the general formula (TEMP-103) is represented by the following general formula (TEMP-104).

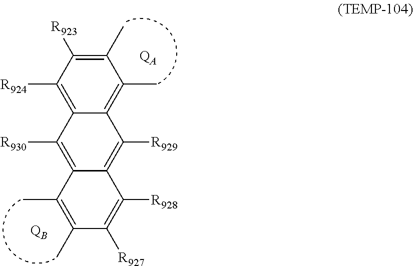

The case where the “set of adjacent two or more” form a ring includes not only the case where the set (pair) of adjacent “two” is bonded with as in the above-mentioned examples, but also the case where the set of adjacent “three or more” are bonded with each other. For example, it means the case where R₉₂₁ and R₉₂₂ form a ring Q_{A} by bonding with each other, and R₉₂₂ and R₉₂₃ form a ring Q_{c} by bonding with each other, and adjacent three (R₉₂₁, R₉₂₂ and R₉₂₃) form rings by bonding with each other and together fused to the anthracene mother skeleton. In this case, the anthracene compound represented by the general formula (TEMP-103) is represented by the following general formula (TEMP-105). In the following general formula (TEMP-105), the ring Q_{A} and the ring Q_{c} share R₉₂₂.

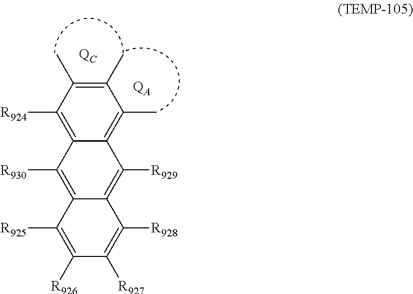

The “monocycle” or “fused ring” formed may be a saturated ring or an unsaturated ring, as a structure of the formed ring alone. Even when the “one pair of adjacent two” forms a “monocycle” or a “fused ring”, the “monocycle” or the “fused ring” may form a saturated ring or an unsaturated ring. For example, the ring Q_{A} and the ring Q_{B} formed in the general formula (TEMP-104) are independently a “monocycle” or a “fused ring.” The ring Q_{A} and the ring Q_{c} formed in the general formula (TEMP-105) are “fused ring.” The ring Q_{A} and ring Q_{c} of the general formula (TEMP-105) are fused ring by fusing the ring Q_{A} and the ring Q_{c} together. When the ring Q_{A} of the general formula (TMEP-104) is a benzene ring, the ring QA is a monocycle. When the ring Q_{A} of the general formula (TMEP-104) is a naphthalene ring, the ring Q_{A} is a fused ring.

The “unsaturated ring” includes, in addition to an aromatic hydrocarbon ring and an aromatic heterocycle, an aliphatic hydrocarbon ring with an unsaturated bond, i.e., double and/or triple bonds in the ring structure (e.g., cyclohexene, cyclohexadiene, etc., and a non-aromatic heterocycle with an unsaturated bond (e.g., dihydropyran, imidazoline, pyrazoline, quinolizine, indoline, isoindoline, etc.). The “saturated ring” includes an aliphatic hydrocarbon ring without an unsaturated bond and a non-aromatic heterocycle without ab unsaturated bond.

Specific examples of the aromatic hydrocarbon ring include a structure in which the group listed as a specific example in the specific example group G1 is terminated by a hydrogen atom.

Specific examples of the aromatic heterocycle include a structure in which the aromatic heterocyclic group listed as a specific example in the example group G2 is terminated by a hydrogen atom.

Specific examples of the aliphatic hydrocarbon ring include a structure in which the group listed as a specific example in the specific example group G6 is terminated by a hydrogen atom.

The term “to form a ring” means forming a ring only with plural atoms of the mother skeleton, or with plural atoms of the mother skeleton and one or more arbitrary atoms in addition. For example, the ring Q_{A} shown in the general formula (TEMP-104), which is formed by bonding R₉₂₁ and R₉₂₂ with each other, is a ring formed from the carbon atom of the anthracene skeleton with which R₉₂₁ is bonded, the carbon atom of the anthracene skeleton with which R₉₂₂ is bonded, and one or more arbitrary atoms. For example, in the case where the ring Q_{A} is formed with R₉₂₁ and R₉₂₂, when a monocyclic unsaturated ring is formed with the carbon atom of the anthracene skeleton with which R₉₂₁ is bonded, the carbon atom of the anthracene skeleton with which R₉₂₂ is bonded, and four carbon atoms, the ring formed with R₉₂₁ and R₉₂₂ is a benzene ring.

Here, the “arbitrary atom” is preferably at least one atom selected from the group consisting of a carbon atom, a nitrogen atom, an oxygen atom, and a sulfur atom, unless otherwise specified in this specification. In the arbitrary atom (for example, a carbon atom or a nitrogen atom), a bond which does not form a ring may be terminated with a hydrogen atom or the like, or may be substituted with “arbitrary substituent” described below. When an arbitrary atom other than a carbon atom is contained, the ring formed is a heterocycle.

The number of “one or more arbitrary atom(s)” constituting a monocycle or a fused ring is preferably 2 or more and 15 or less, more preferably 3 or more and 12 or less, and still more preferably 3 or more and 5 or less, unless otherwise specified in this specification.

The “monocycle” is preferable among the “monocycle” and the “fused ring”, unless otherwise specified in this specification.

The “unsaturated ring” is preferable among the “saturated ring” and the “unsaturated ring”, unless otherwise specified in this specification.

Unless otherwise specified in this specification, the “monocycle” is preferably a benzene ring.

Unless otherwise specified in this specification, the “unsaturated ring” is preferably a benzene ring.

Unless otherwise specified in this specification, when “one or more sets of adjacent two or more” are “bonded with each other to form a substituted or unsubstituted monocycle” or “bonded with each other to form a substituted or unsubstituted fused ring”, this specification, one or more sets of adjacent two or more are preferably bonded with each other to form a substituted or unsubstituted “unsaturated ring” from plural atoms of the mother skeleton and one or more and 15 or less atoms which is at least one kind selected from a carbon atom, a nitrogen atom, an oxygen atom, and a sulfur atom.

The substituent in the case where the above-mentioned “monocycle” or “fused ring” has a substituent is, for example, an “arbitrary substituent” described below. Specific examples of the substituent which the above-mentioned “monocycle” or “fused ring” has include the substituent described above in the “Substituent described in this specification” section.

The substituent in the case where the above-mentioned “saturated ring” or “unsaturated ring” has a substituent is, for example, an “arbitrary substituent” described below. Specific examples of the substituent which the above-mentioned “monocycle” or “fused ring” has include the substituent described above in the “Substituent described in this specification” section.

The foregoing describes the case where “one or more sets of adjacent two or more form a substituted or unsubstituted monocycle by bonding with each other” and the case where “one or more sets of adjacent two or more form a substituted or unsubstituted fused ring by bonding with each other ” (the case where “forming a ring by bonding with each other”).

Substituent in the case of “substituted or unsubstituted” In one embodiment in this specification, the substituent (in this specification, sometimes referred to as an “arbitrary substituent”) in the case of “substituted or unsubstituted” is, for example, a group selected from the group consisting of:
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₀)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, and
an unsubstituted heterocyclic group including 5 to 50 ring atoms,
wherein, R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted heterocyclic group including 5 to 50 ring atoms.
When two or more R₉₀₀'s are present, the two or more R₉₀₀'s may be the same or different.
When two or more R₉₀₂'s are present, the two or more R₉₀₂'s may be the same or different.
When two or more R₉₀₀'s are present, the two or more R₉₀₃'s may be the same or different.
When two or more R₉₀₄'s are present, the two or more R₉₀₄'s may be the same or different.
When two or more R₉₀₅'s are present, the two or more R₉₀₅'s may be the same or different.
When two or more R₉₀₆'s are present, the two or more R₉₀₆'s may be the same or different.
When two or more R₉₀₇'s are present, the two or more R₉₀₇'s may be the same or different.

In one embodiment, the substituent in the case of “substituted or unsubstituted” is a group selected from the group consisting of:
an alkyl group including 1 to 50 carbon atoms,
an aryl group including 6 to 50 ring carbon atoms, and
a heterocyclic group including 5 to 50 ring atoms.

In one embodiment, the substituent in the case of “substituted or unsubstituted” is a group selected from the group consisting of:
an alkyl group including 1 to 18 carbon atoms,
an aryl group including 6 to 18 ring carbon atoms, and
a heterocyclic group including 5 to 18 ring atoms.

Specific examples of each of the arbitrary substituents include specific examples of substituent described in the section “Substituent described in this specification” above.

Unless otherwise specified in this specification, adjacent arbitrary substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, more preferably form a benzene ring.

Unless otherwise specified in this specification, the arbitrary substituent may further have a substituent. The substituent which the arbitrary substituent further has is the same as that of the above-mentioned arbitrary substituent.

In this specification, the numerical range represented by “AA to BB” means the range including the numerical value AA described on the front side of “AA to BB” as the lower limit and the numerical value BB described on the rear side of “AA to BB” as the upper limit.

### [Novel Compound]

A compound according to an aspect of the present invention is represented by the following formula (1):

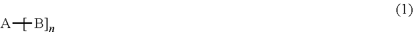

wherein in the formula (1),
A is
a substituted or unsubstituted aromatic hydrocarbon ring group containing a nitrogen atom as a part of a substituent and having a fused ring composed of 5 or more rings, or
a substituted or unsubstituted heterocyclic group containing a nitrogen atom as a part of the skeleton or a substituent and having a fused ring composed of 5 or more rings;
B is a monovalent group represented by the following formula (b1):

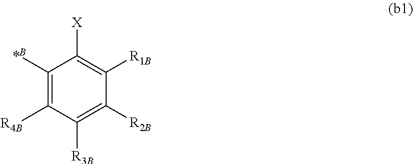

wherein in the formula (b1),
*B is bonded with the nitrogen atom contained as a part of the skeleton of A or a substituent via a single bond;
one or more sets of the adjacent two or more of R_{1B} to R_{4B} form an unsubstituted saturated ring, or a substituted or unsubstituted, unsaturated ring by bonding with each other;
R_{1B} to R_{4B} which do not form the unsubstituted saturated ring, or the substituted or unsubstituted, unsaturated ring are independently a hydrogen atom or a substituent R;
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₀)(R₉₀₂)(R₉₀₃),
—O—(R₉₀₄),
—S—(R₉₀₅),
—N(R₉₀₆)(R₉₀₇)
(wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms;
when two or more substituents R are present, the two or more substituents R may be the same as or different from each other;
X is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₀)(R₉₀₂)(R₉₀₃),
—O—(R₉₀₄),
—S—(R₉₀₅),
—N(R₉₀₆)(R₉₀₇),
a halogen atom, a cyano group, a nitro group, and
a substituted or unsubstituted aryl group having 6 to 50 ring carbon atoms;
R₉₀₁ to R₉₀₇ are the same as defined in the substituent R; and
n is an integer of 1 to 5; when n is 2 or more, two or more B's may be the same as or different from each other.

In the compound according to an aspect of the present invention, as understood from the above-described definition of the formula (1), the nitrogen atom contained in A and X are bonded with relatively bulky aromatic group such as a naphthalene and a tetralin at ortho-position. As a result, a side reaction can be suppressed by sterically protecting the nitrogen atom in which it is presumed that reactivity is increased, since the density of positive charge is increased in emission of an organic EL device.

Generally, in the compound using a group in which a substituent is bonded with the nitrogen atom at ortho-position, in order to sterically protect the nitrogen atom as described above, the emission wavelength from the organic EL device is shifted to short wavelength side, and then apparent efficiency thereof is decreased by decreasing visibility thereof. Further, it has been concerned that one's photoreceptor cell is adversely affected by enhancing emission energy.

When the compound according to an aspect of the present invention, an organic EL device having high chromatic purity and long lifetime without shortening emission wavelength therefrom can be fabricated.

The fused ring composed of 5 or more rings is not particularly limited, as long as it is a ring formed by fusing 5 or more single rings.

For example, when four benzene rings and one furan ring are fused, the following dinaphtho[2,3-b:2′,3′-d]furan ring is formed.

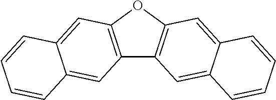 [SMILES: c1ccc2cc3c(cc2c1)oc1cc2ccccc2cc13]

A style in fusing rings each other is not particularly limited, and when four benzene rings and one furan ring are fused, the following dinaphtho[2,1-b:1′,2′-d]furan ring may be formed, or

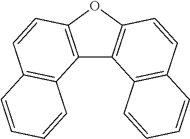 [SMILES: c1ccc2c(c1)ccc1oc3ccc4ccccc4c3c12]

the following dinaphtho[1,2-b:1′,2′-d]furan ring may be formed.

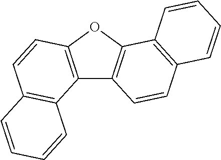 [SMILES: c1ccc2c(c1)ccc1c2oc2ccc3ccccc3c21]

Further, when three benzene rings and two 1,4-azaborine rings are fused, for example, the following ring is formed.

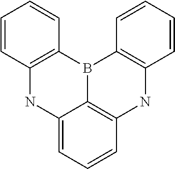 [SMILES: c1ccc2c(c1)Nc1cccc3c1B2c1ccccc1N3]

Furthermore, when three benzene rings, one 1,4-azaborine ring and one 1,2-azaborine ring are fused, for example, the following ring is formed.

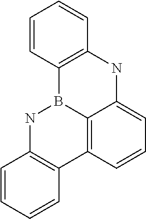 [SMILES: c1ccc2c(c1)Nc1cccc3c1B2Nc1ccccc1-3]

The expression “containing a nitrogen atom as a part of a substituent” means that the “fused ring composed of 5 or more rings” includes at least one substituent containing at least one nitrogen atom. The same can be applied to the expression “containing a nitrogen atom as a part of a substituent” in the case of the expression “containing a nitrogen atom as a part of the skeleton or a substituent”.

The expression “containing a nitrogen atom as a part of the skeleton” in the case of the expression “containing a nitrogen atom as a part of the skeleton or a substituent” means that at least one of 5 or more single rings constituting the “fused ring composed of 5 or more rings” contains at least one nitrogen atom.

As understood from the above-described definition of the formula (1), when n is 2 or more in the compound represented by the formula (1), each of two or more B's is directly bonded with the nitrogen atom contained in A.

In one embodiment, n is 1 or 2.

In one embodiment, A is a group selected from groups represented by the following formulas (a-1) to (a-9):

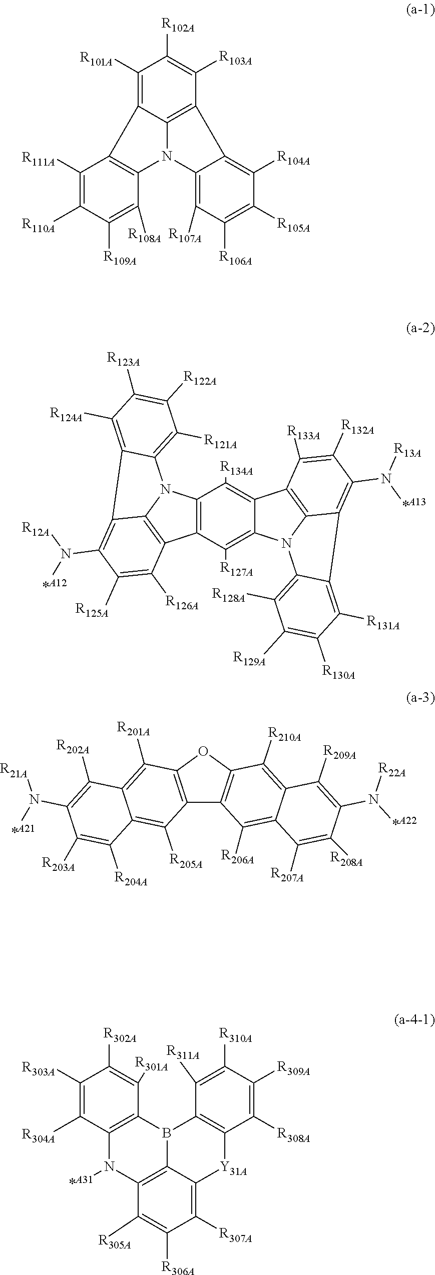

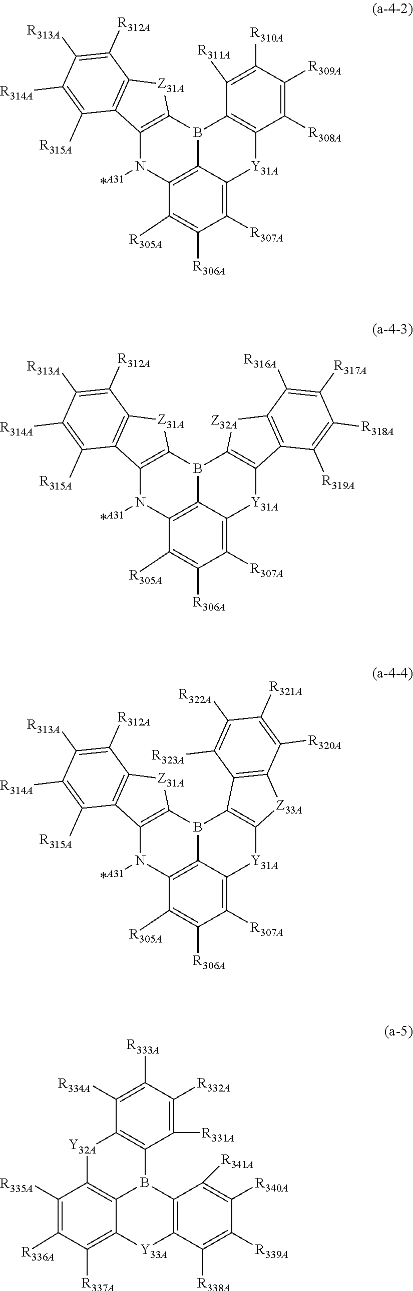

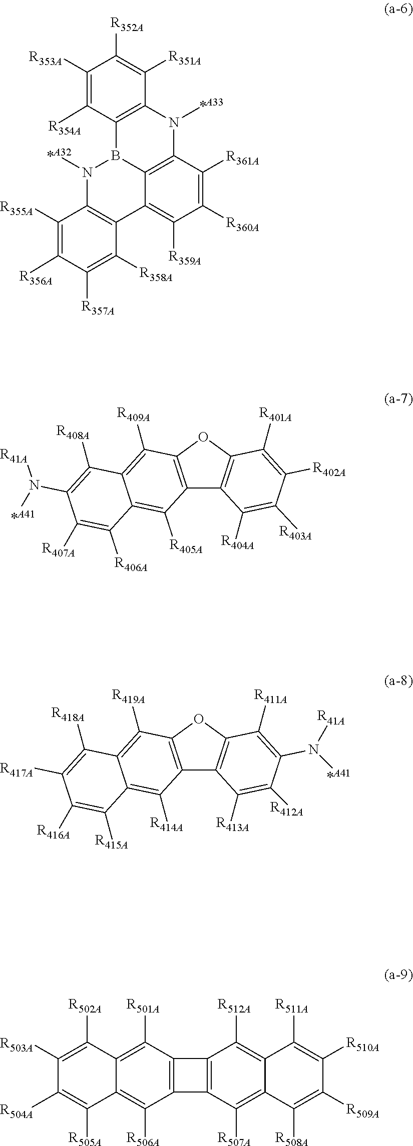

wherein in the formula (a-1),

at least one of R_{101A} to R_{111A} is a group represented by the following formula (a-11):

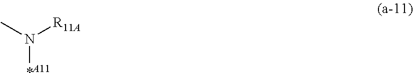

wherein in the formula (a-11),
*A11 is a bonding position in which *B in the formula (b1) is bonded therewith;
R_{11A} is a substituent R; R_{101A} to R_{111A} which are not the group represented by the formula (a-11) are independently
a hydrogen atom or a substituent R;
when two or more groups represented by the formula (a-11) are present, the two or more groups represented by the formula (a-11) may be the same as or different from each other;
in the formula (a-2),
at least one of *A12 and *A13 is a bonding position in which *B in the formula (b1) is bonded therewith;
*A12 and *A13 which is not the bonding position in which *B in the formula (b1) is bonded therewith is a substituent R;
R_{12A} and R_{13A} are a substituent R;
R_{121A} to R_{134A} are independently a hydrogen atom or a substituent R;
in the formula (a-3),
at least one of *A21 and *A22 is a bonding position in which *B in the formula (b1) is bonded therewith;
*A21 and *A22 which is not the bonding position in which *B in the formula (b1) is bonded
therewith is a substituent R;
R_{21A} and R_{22A} are a substituent R;
R_{201A} to R_{210A} are independently a hydrogen atom or a substituent R;
in the formula (a-4-1),
A31 is a bonding position in which *B in the formula (b1) is bonded therewith;
R_{301A} to R_{311A} are independently a hydrogen atom or a substituent R;
Y_{31A} is C(R₁₁)(R₁₂), N(R₁₃), Si(R₁₄)(R₁₅), O, or S;
R₁₁ to R₁₅ are independently a hydrogen atom or a substituent R;
in the formula (a-4-2),
A31 is a bonding position in which *B in the formula (b1) is bonded therewith;
R_{305A} to R_{315A} are independently a hydrogen atom or a substituent R;
Y_{31A} is C(R₁₁)(R₁₂), N(R₁₃), Si(R₁₄)(R₁₅), O, or S;
Z_{31A} is C(R₁₁)(R₁₂), O, or S;
R₁₁ to R₁₅ are independently a hydrogen atom or a substituent R;
when two or more of each of R₁₁ and R₁₂ are present, the two or more of each of R₁₁ and R₁₂ may be the same as or different from each other;
in the formula (a-4-3),
A31 is a bonding position in which *B in the formula (b1) is bonded therewith;
R_{305A} to R_{307A} and R_{312A} to R_{319A} are independently a hydrogen atom or a substituent R;
Y_{31A} is C(R₁₁)(R₁₂), N(R₁₃), Si(R₁₄)(R₁₅), O, or S;
Z_{31A} and Z_{32A} are independently C(R₁₁)(R₁₂), O, or S;
R₁₁ to R₁₅ are independently a hydrogen atom or a substituent R;
when two or more of each of R₁₁ and R₁₂ are present, the two or more of each of R₁₁ and R₁₂ may be the same as or different from each other;
in the formula (a-4-4),
*A31 is a bonding position in which *B in the formula (b1) is bonded therewith;
R_{305A} to R_{307A}, R_{312A} to R_{315A}, and R_{320A} to R_{323A} are independently a hydrogen atom or a substituent R;
Y_{31A} is C(R₁₁)(R₁₂), N(R₁₃), Si(R₁₄)(R₁₅), O, or S;
Z_{31A} and Z_{33A} are independently C(R₁₁)(R₁₂), O, or S;
R₁₁ to R₁₅ are independently a hydrogen atom or a substituent R;
when two or more of each of R₁₁ and R₁₂ are present, the two or more of each of R₁₁ and R₁₂ may be the same as or different from each other;
in the formula (a-5),
at least one of R_{331A} to R_{341A} is the group represented by the formula (a-11);
R_{331A} to R_{341A} which are not the group represented by the formula (a-11) are independently a hydrogen atom or a substituent R;
when two or more groups represented by the formula (a-11) are present, the two or more groups represented by the formula (a-11) may be the same as or different from each other;
Y_{32A} and Y_{33A} are independently C(R₁₁)(R₁₂), N(R₁₃), Si(R₁₄)(R₁₅), O, or S;
R₁₁ to R₁₅ are independently a hydrogen atom or a substituent R;
when two or more of each of R₁₁ to R₁₅ are present, the two or more of each of R₁₁ to R₁₅ may be the same as or different from each other;
in the formula (a-6),
at least one of *A32 and *A33 is a bonding position in which *B in the formula (b1) is bonded therewith;
*A32 and *A33 which is not the bonding position in which *B in the formula (b1) is bonded therewith is a substituent R;
R_{351A} to R_{361A} are independently a hydrogen atom or a substituent R; in the formula (a-7),
*A41 is a bonding position in which *B in the formula (b1) is bonded therewith, or a substituent R;
R_{41A} is a substituent R;
the adjacent two of R_{401A} to R_{404 A} form a group represented by the following formula (a-12) or (a-13) by bonding with each other:

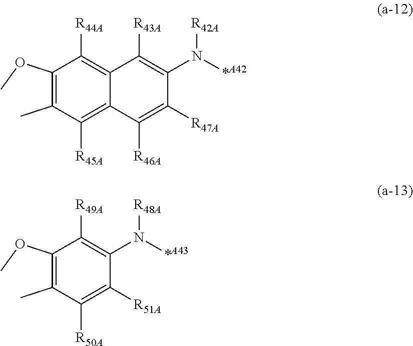

wherein in the formula (a-12),
*A42 is a bonding position in which *B in the formula (b1) is bonded therewith, or a substituent R;
R_{42A} is a substituent R;
R_{43A} to R_{47A} are independently a hydrogen atom or a substituent R;
in the formula (a-13),
*A43 is a bonding position in which *B in the formula (b1) is bonded therewith, or a substituent R;
R_{48A} is a substituent R;
R_{49A} to R_{51A} are independently a hydrogen atom or a substituent R;
when the adjacent two of R_{401A} to R_{404 A} form the group represented by the formula (a-12) by bonding with each other, at least one of *A41 and *A42 is a bonding position in which *B in the formula (b1) is bonded therewith;
when the adjacent two of R_{401A} to R_{404 A} form the group represented by the formula (a-13) by bonding with each other, at least one of *A41 and *A43 is a bonding position in which *B in the formula (b1) is bonded therewith;
R_{405A} to R_{409A}, and R_{401A} to R_{404A} which do not form the group represented by the formula (a-12) or (a-13) are independently a hydrogen atom or a substituent R;
in the formula (a-8),
*A41 is a bonding position in which *B in the formula (b1) is bonded therewith, or a substituent R;
R_{41A} is a substituent R;
the adjacent two of R_{415A} to R_{418A} form the group represented by the formula (a-12) or (a-13) by bonding with each other;
when the adjacent two of R_{415A} to R_{418A} form the group represented by the formula (a-12) by bonding with each other, at least one of *A41 and *A42 is a bonding position in which *B in the formula (b1) is bonded therewith;
when the adjacent two of R_{415A} to R_{418A} form the group represented by the formula (a-13) by bonding with each other, at least one of *A41 and *A43 is a bonding position in which *B in the formula (b1) is bonded therewith; R_{411A} to R_{414A}, R_{419A}, and R_{415A} to R_{418A} which do not form the group represented by the formula (a-12) or (a-13) are independently a hydrogen atom or a substituent R; in the formula (a-9),
at least one of R_{501A} to R_{512A} is the group represented by the formula (a-11); R_{501A} to R_{512A} which are not the group represented by the formula (a-11) are independently
a hydrogen atom or a substituent R;
when two or more groups represented by the formula (a-11) are present, the two or more groups represented by the formula (a-11) may be the same as or different from each other; and
the substituent R is the same as defined in the formula (1).

In one embodiment, one or more sets of the adjacent two or more of R_{1B} to R_{4B} form, by bonding with each other,
an unsubstituted saturated hydrocarbon ring having 6 to 12 ring carbon atoms,
a substituted or unsubstituted, unsaturated hydrocarbon ring having 6 to 12 ring carbon atoms,
an unsubstituted saturated heterocycle having 5 to 12 ring atoms, or
a substituted or unsubstituted, unsaturated heterocycle having 5 to 12 ring atoms.

In one embodiment, B is a group selected from groups represented by the following formulas (b2-1) to (b2-15):

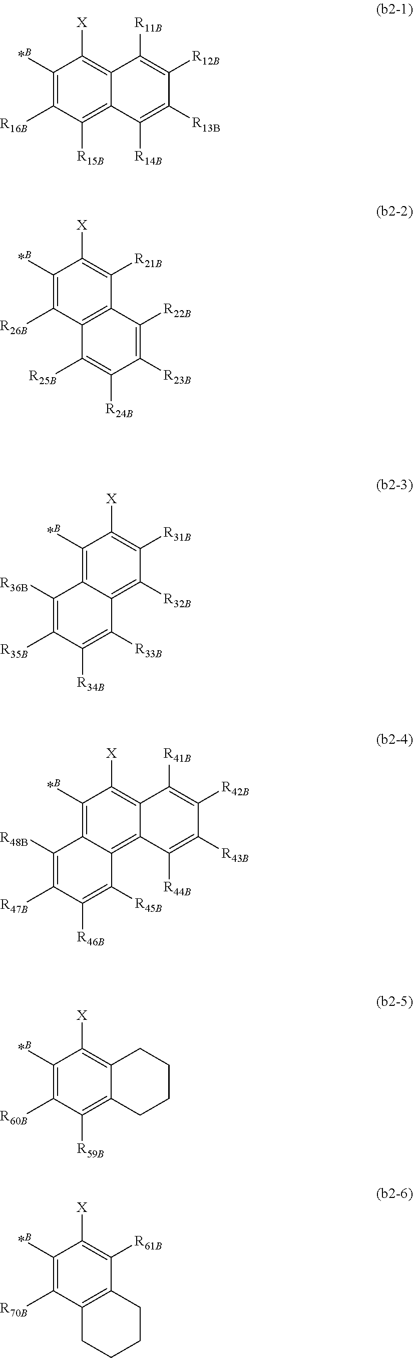

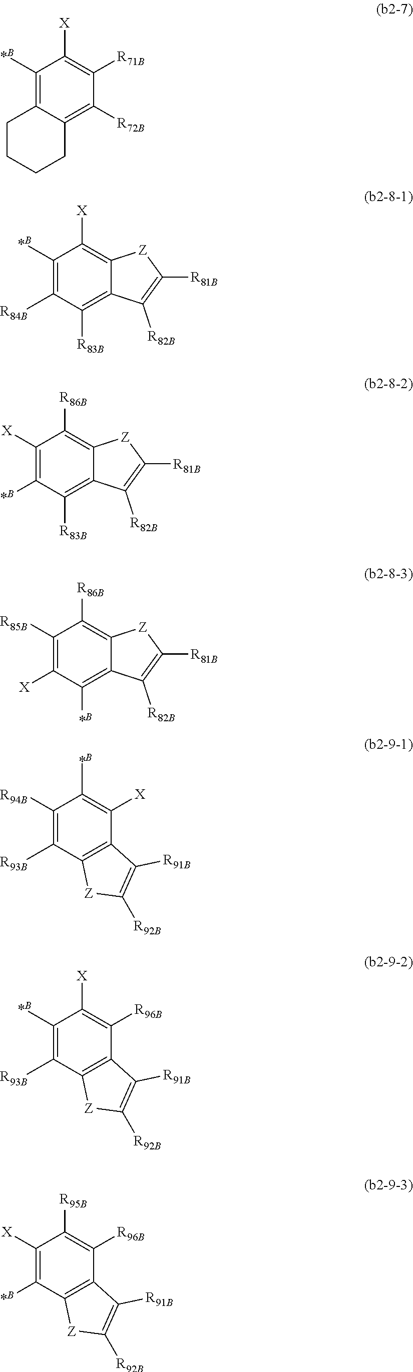

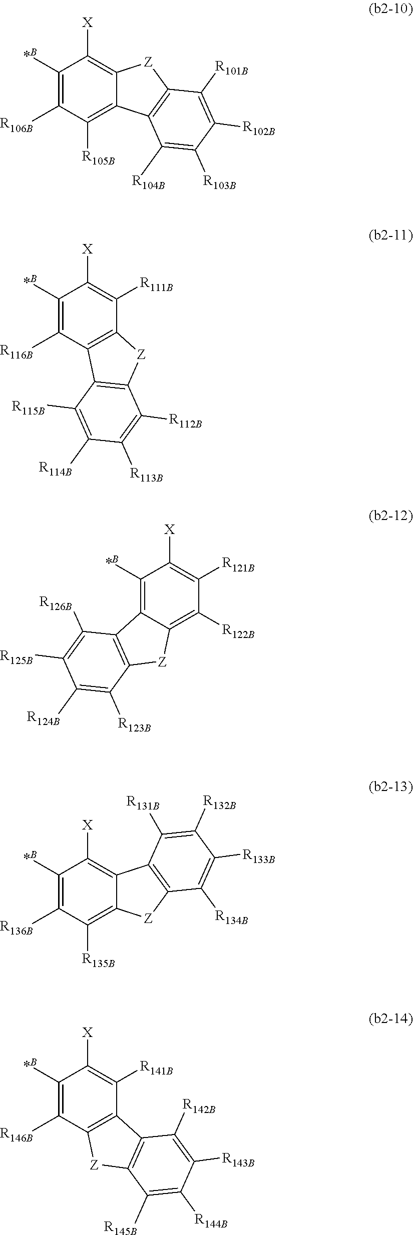

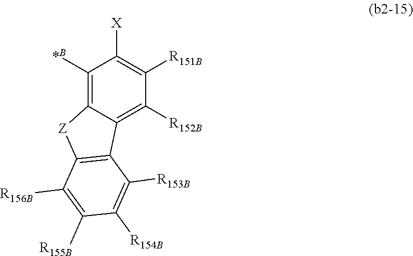

wherein in the formulas (b2-1) to (b2-15),
*B is bonded with the nitrogen atom contained as a part of the skeleton of A or a substituent via a single bond;
R_{11B} to R_{16B}, R_{21B} to R_{26B}, R_{31B} to R_{36B}, R_{41B} to R_{48B}, R_{59B} to R_{60B}, R_{61B}, R_{70B}, R_{71B} to R_{72B}, R_{81B} to R_{86B}, R_{91B} to R_{96B}, R_{101B} to R_{106B}, R_{111B} to R_{116B}, R_{121B} to R_{126B}, R_{131B} to R_{136B}, R_{141B} to R_{146B}, and R_{151B} to R_{156B} are independently a hydrogen atom or a substituent R;
Z is O, S, or C(R₁₁)(R₁₂);
R₁₁ and R₁₂ are a substituent R; and
X and the substituent R is the same as defined in the formula (1).

In one embodiment, the compound represented by the formula (1) is represented by the following formula (1-1):

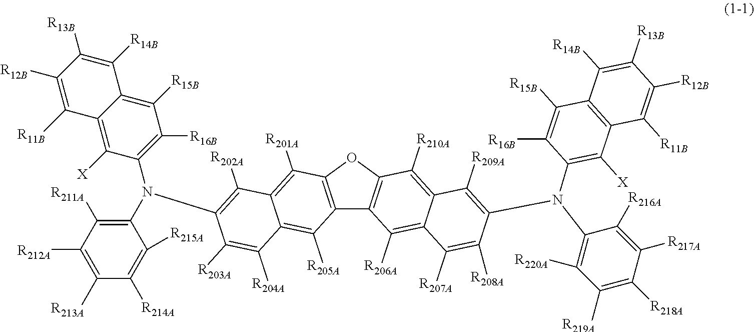

wherein in the formula (1-1),
R_{201A} to R_{220A} and R_{11B} to R_{16B} are independently a hydrogen atom or a substituent R;
X and the substituent R is the same as defined in the formula (1); and
each of the two X's and the two R_{11B}'s to the two R_{16B}'s may be the same as or different from each other.

In one embodiment, the compound represented by the formula (1) is represented by the following formula (1-2):

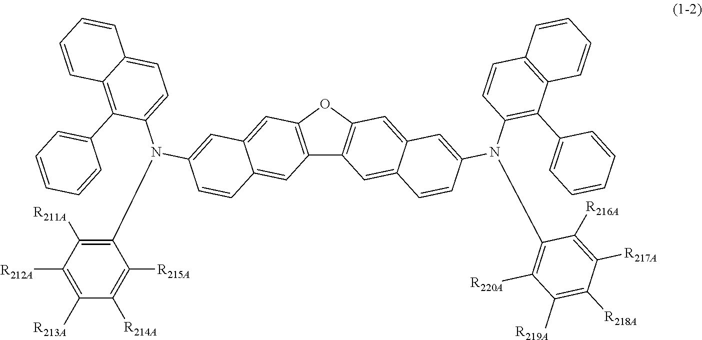

wherein in the formula (1-2),
R_{211A} to R_{220A} are independently a hydrogen atom or a substituent R; and
the substituent R is the same as defined in the formula (1).

In one embodiment, the compound represented by the formula (1) is represented by the following formula (2-1):

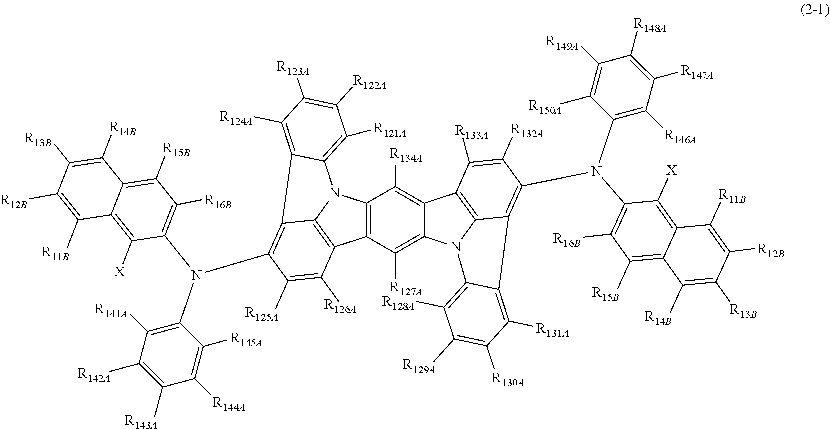

wherein in the formula (2-1),
R_{121A} to R_{134A}, R_{141A} to R_{150A}, and R_{11B} to R_{16B} are independently a hydrogen atom or a substituent R;
X and the substituent R is the same as defined in the formula (1); and
each of the two X's and the two R_{11B}'s to the two R_{16B}'s may be the same as or different from each other.

In one embodiment, the compound represented by the formula (1) is represented by the following formula (2-2):

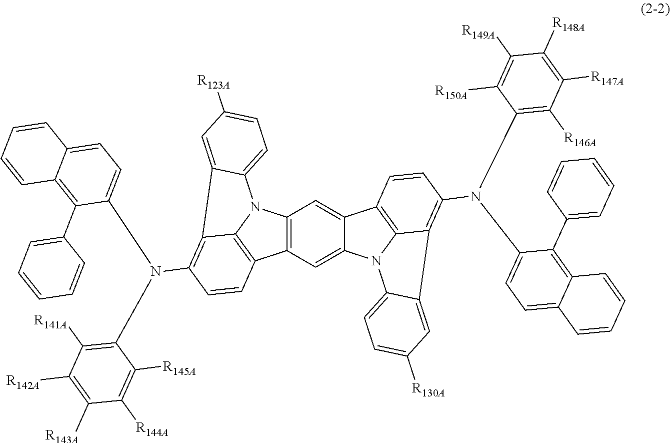

wherein in the formula (2-2),
R_{123A}, R_{130A}, and R_{141A} to R_{150A} are independently a hydrogen atom or a substituent R; and
the substituent R is the same as defined in the formula (1).

In one embodiment, R_{123A} and R_{130A} are a substituted or unsubstituted alkyl group having 1 to 5 carbon atoms. R_{123A} and R_{130A} are may be the same as or different from each other. In one embodiment, R_{123A} and R_{130A} are the same.

In one embodiment, a substituent in the case of “substituted or unsubstituted” in the compound represented by the formula (1) is selected from the group consisting of an alkyl group having 1 to 50 carbon atoms, a haloalkyl group having 1 to 50 carbon atoms, an alkenyl group having 2 to 50 carbon atoms, an alkynyl group having 2 to 50 carbon atoms, a cycloalkyl group having 3 to 50 ring carbon atoms, an alkoxy group having 1 to 50 carbon atoms, an alkylthio group having 1 to 50 carbon atoms, an aryloxy group having 6 to 50 ring carbon atoms, an arylthio group having 6 to 50 ring carbon atoms, an aralkyl group having 7 to 50 carbon atoms, —Si(R₄₁)(R₄₂)(R₄₃), —C(═O)R₄₄, —COOR₄₅, —S(═O)₂R₄₈, —P(═O)(R₄₇)(R₄₈), —Ge(R₄₉)(R₅₀)(R₅₁), —N(R₅₂)(R₅₃) (wherein, R₄₁ to R₅₃ are independently a hydrogen atom, an alkyl group having 1 to 50 carbon atoms, an aryl group having 6 to 50 ring carbon atoms, or a monovalent heterocyclic group having 5 to 50 ring atoms; when two or more of each of R₄₁ to R₅₃ are present, the two or more of each of R₄₁ to R₅₃ may be the same as or different from each other), a hydroxy group, a halogen atom, a cyano group, a nitro group, an aryl group having 6 to 50 ring carbon atoms, and a monovalent heterocyclic group having 5 to 50 ring atoms.

In one embodiment, the substituent in the case of “substituted or unsubstituted” in the compound represented by the formula (1) is an alkyl group having 1 to 50 carbon atoms, an aryl group having 6 to 50 ring carbon atoms, and a monovalent heterocyclic group having 5 to 50 ring atoms.

In one embodiment, the substituent in the case of “substituted or unsubstituted” in the compound represented by the formula (1) is selected from the group consisting of an alkyl group having 1 to 30 carbon atoms, an aryl group having 6 to 30 ring carbon atoms, and a monovalent heterocyclic group having 5 to 30 ring atoms.

In one embodiment, the substituent in the case of “substituted or unsubstituted” in the compound represented by the formula (1) is selected from the group consisting of an alkyl group having 1 to 18 carbon atoms, an aryl group having 6 to 18 ring carbon atoms, and a monovalent heterocyclic group having 5 to 18 ring atoms.

Specific examples of each substituent of the compound represented by the formula (1), the substituent in the case of “substituted or unsubstituted” and the halogen atom are the same as those described above.

The compound represented by the formula (1) can be synthesized in accordance with Examples by using known alternative reactions or raw materials adapted to the target compound.

Specific examples of the compound represented by the formula (1) will be described below, but these are merely examples, and the compound represented by the formula (1) is not limited to the following specific examples.

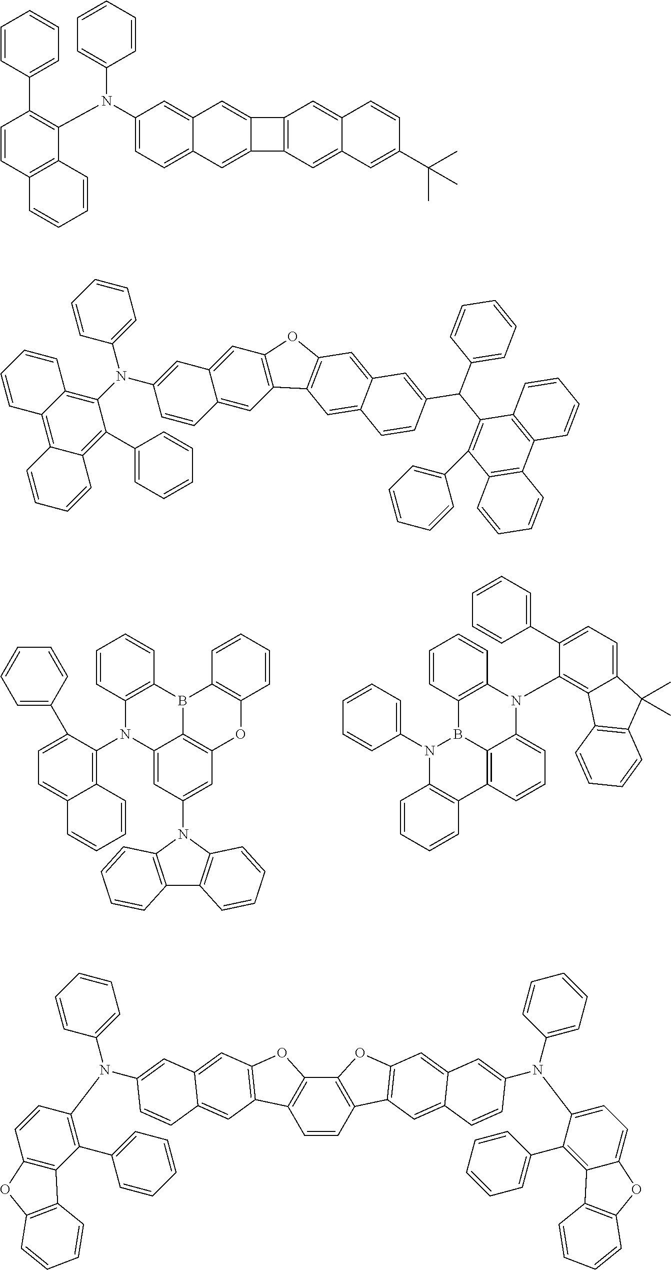

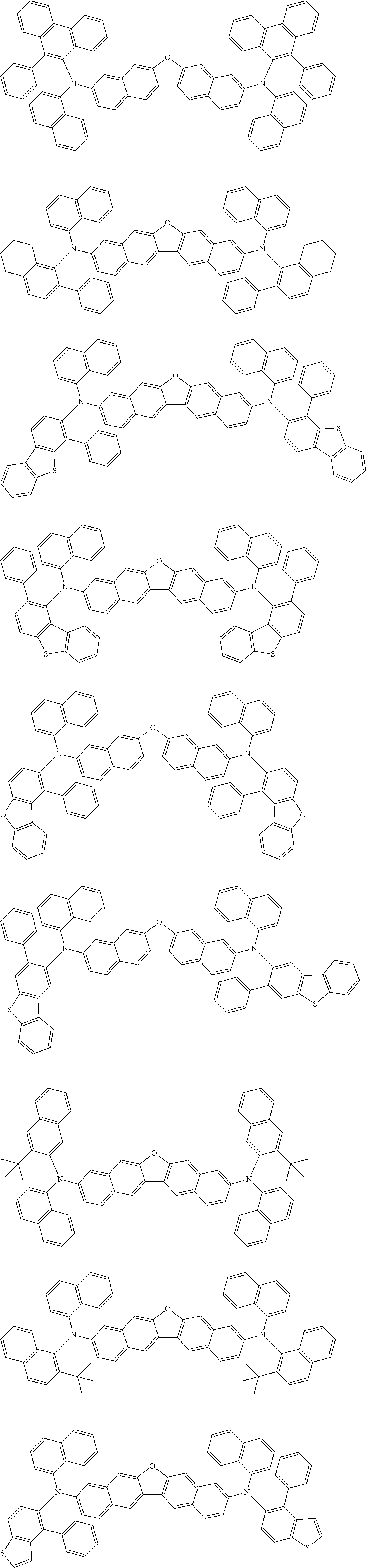

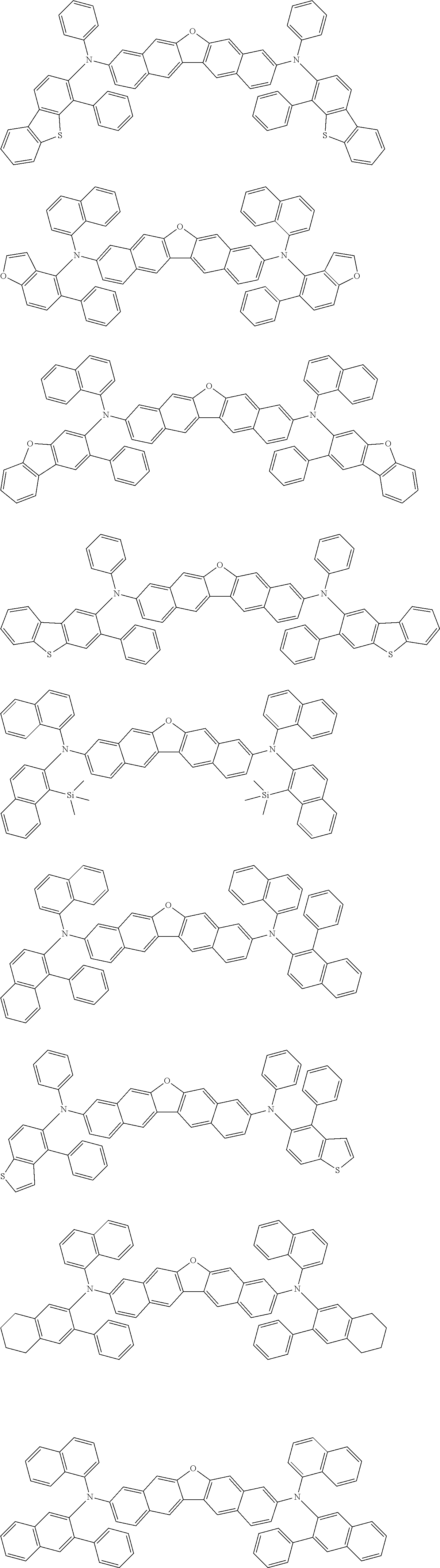

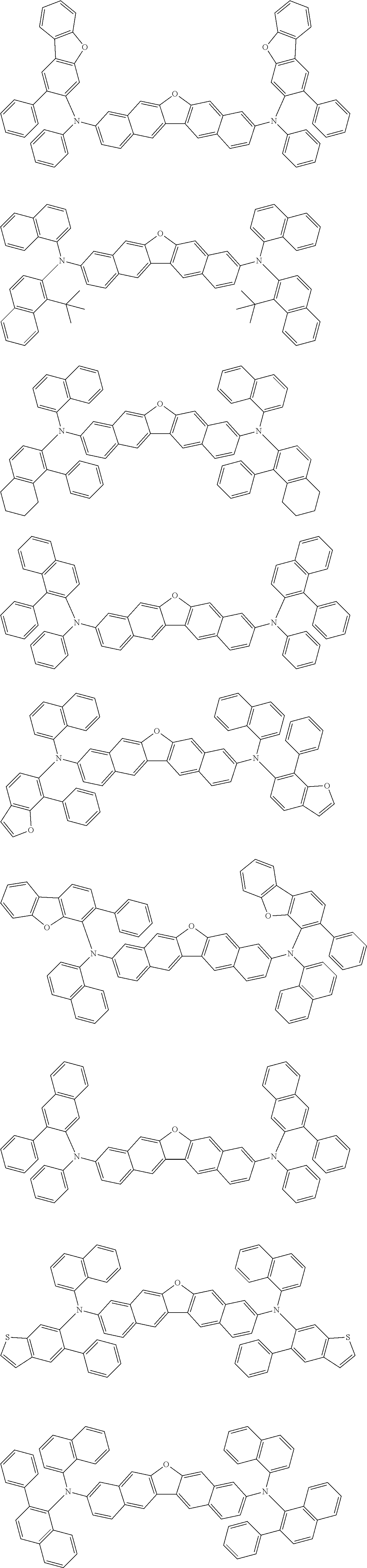

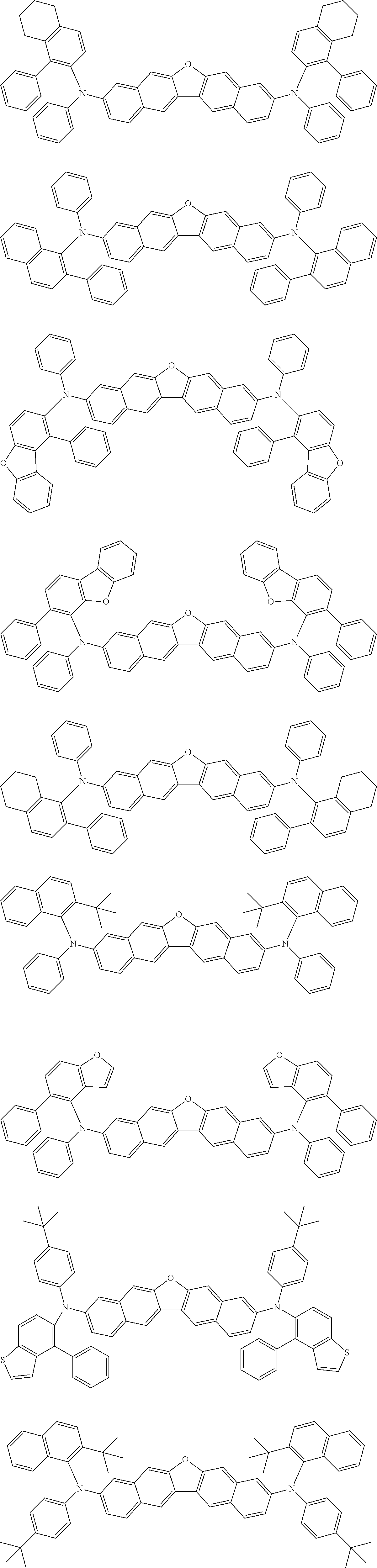

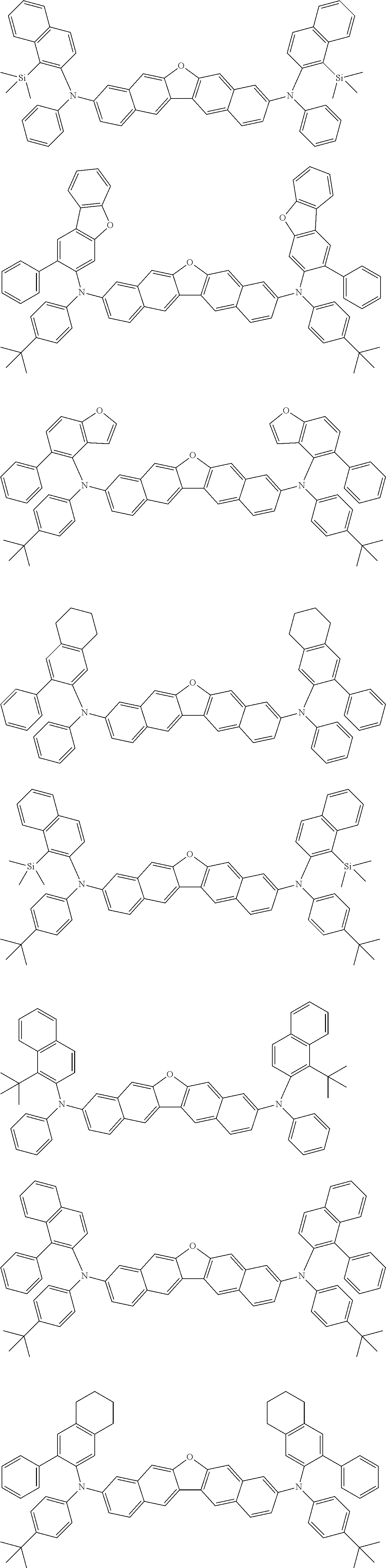

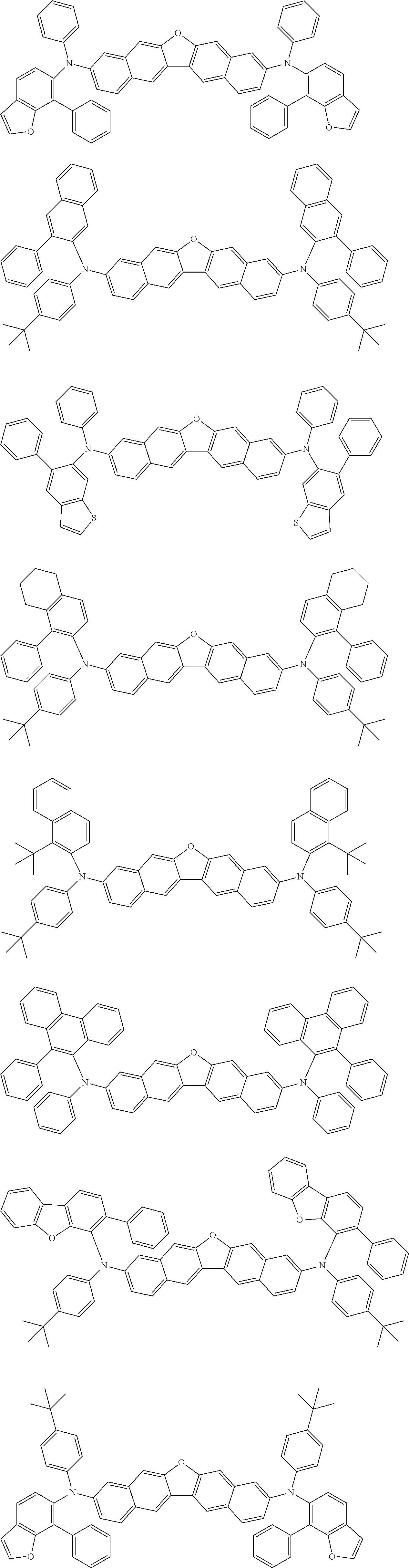

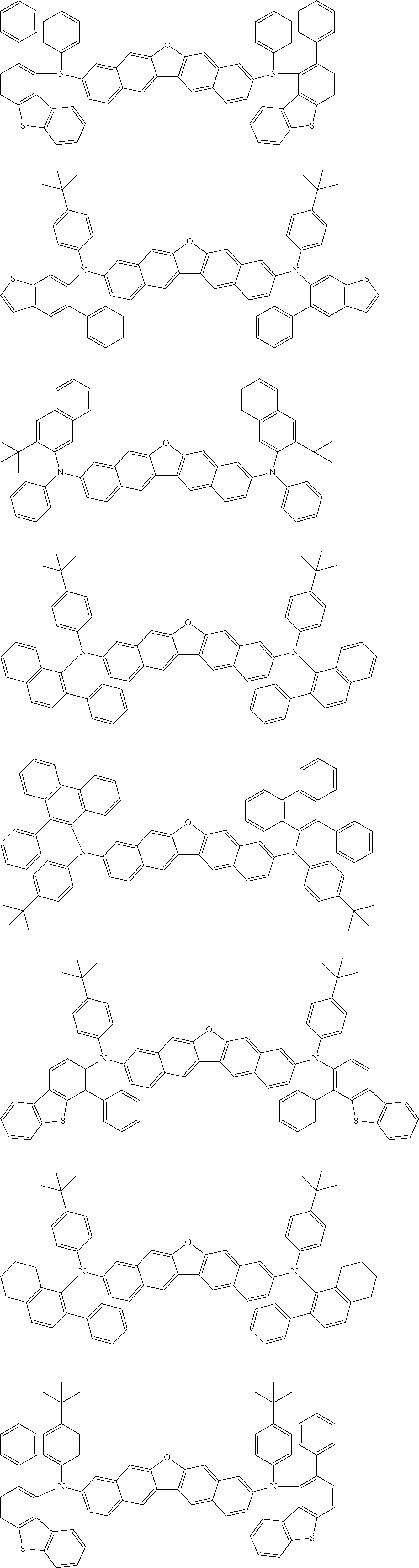

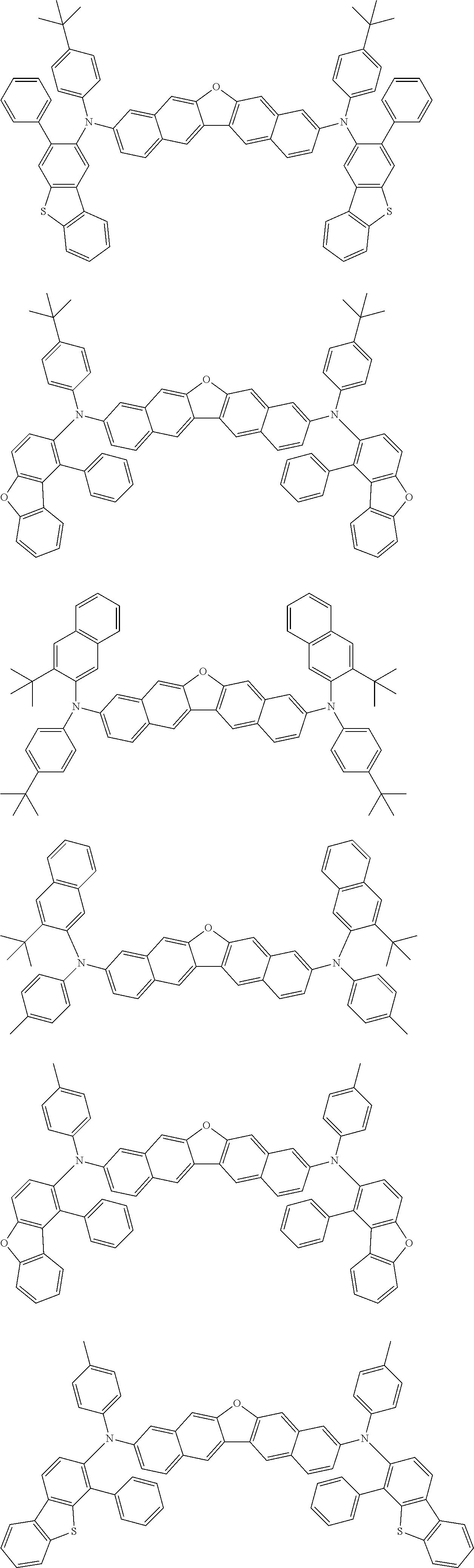

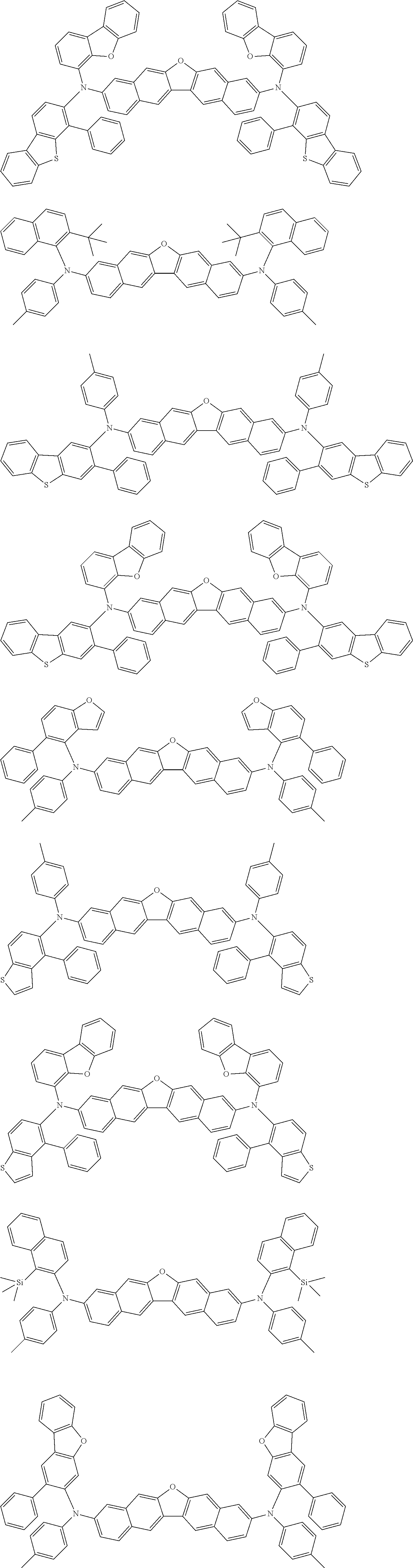

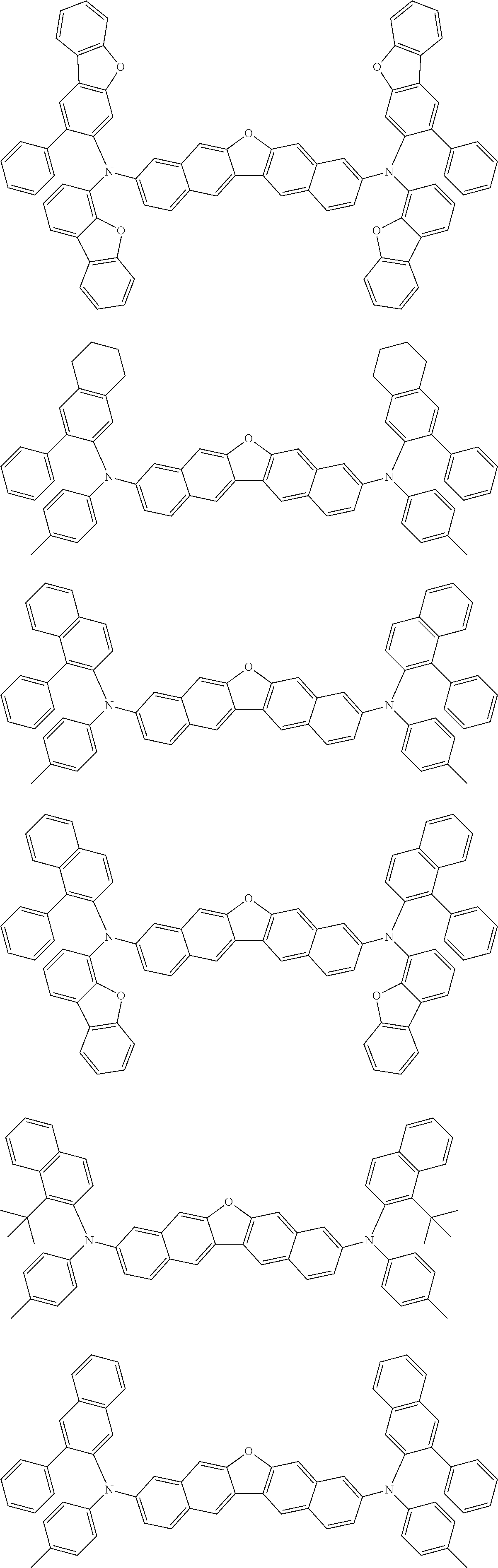

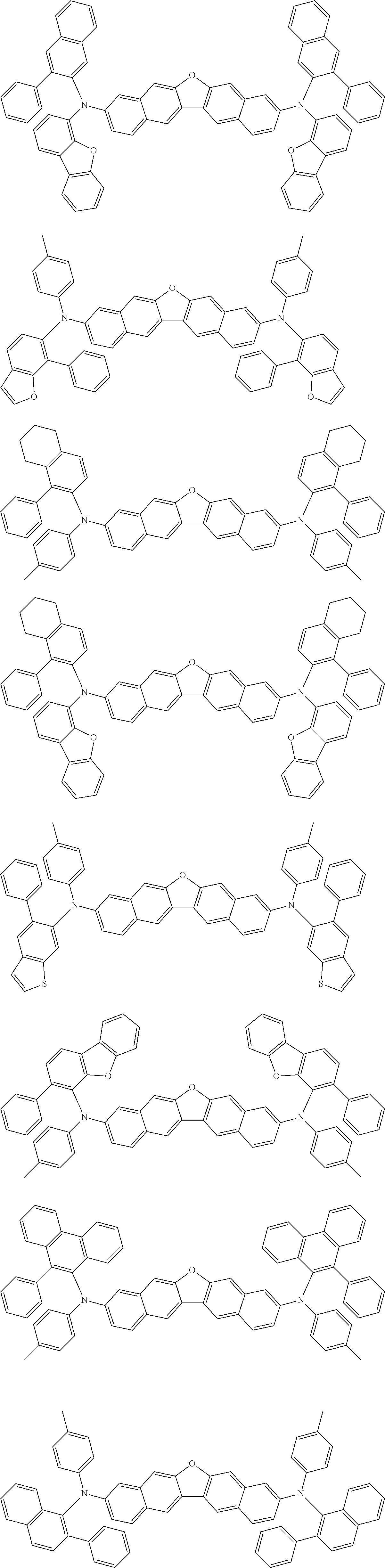

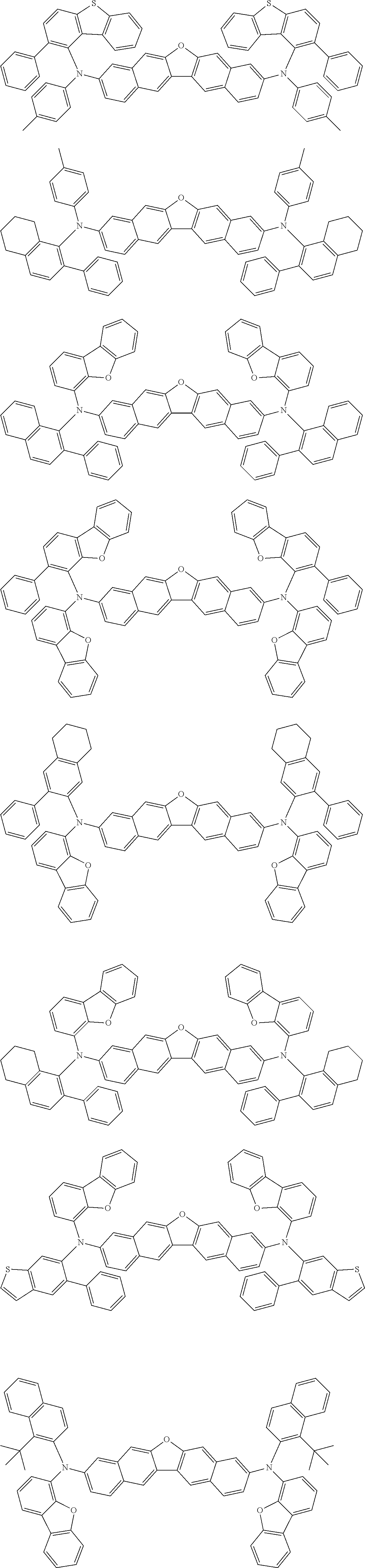

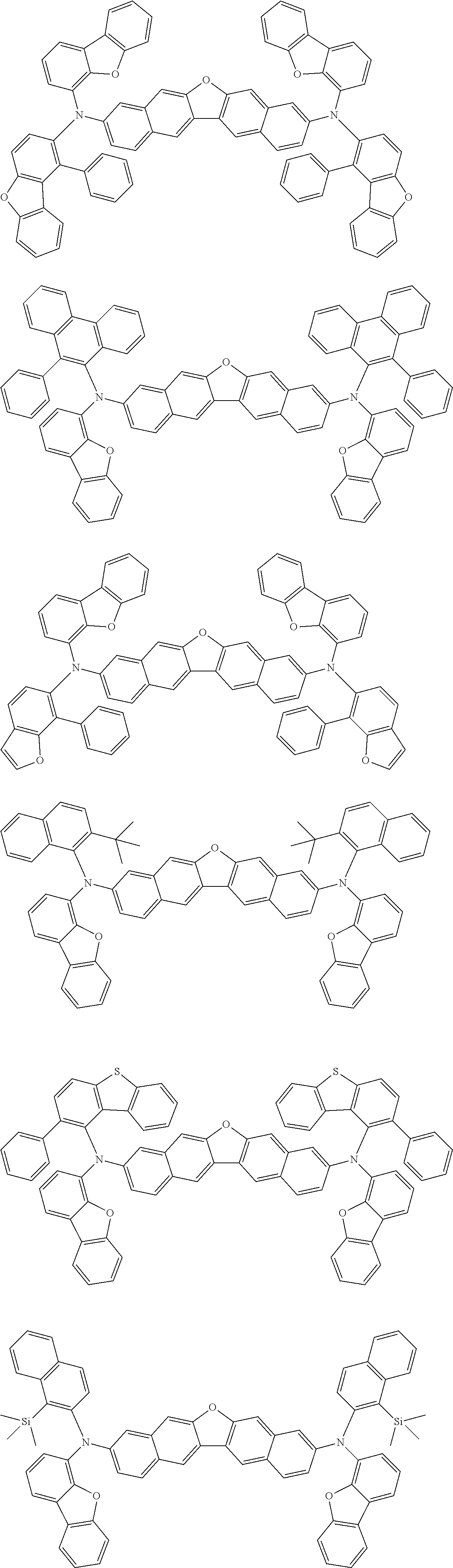

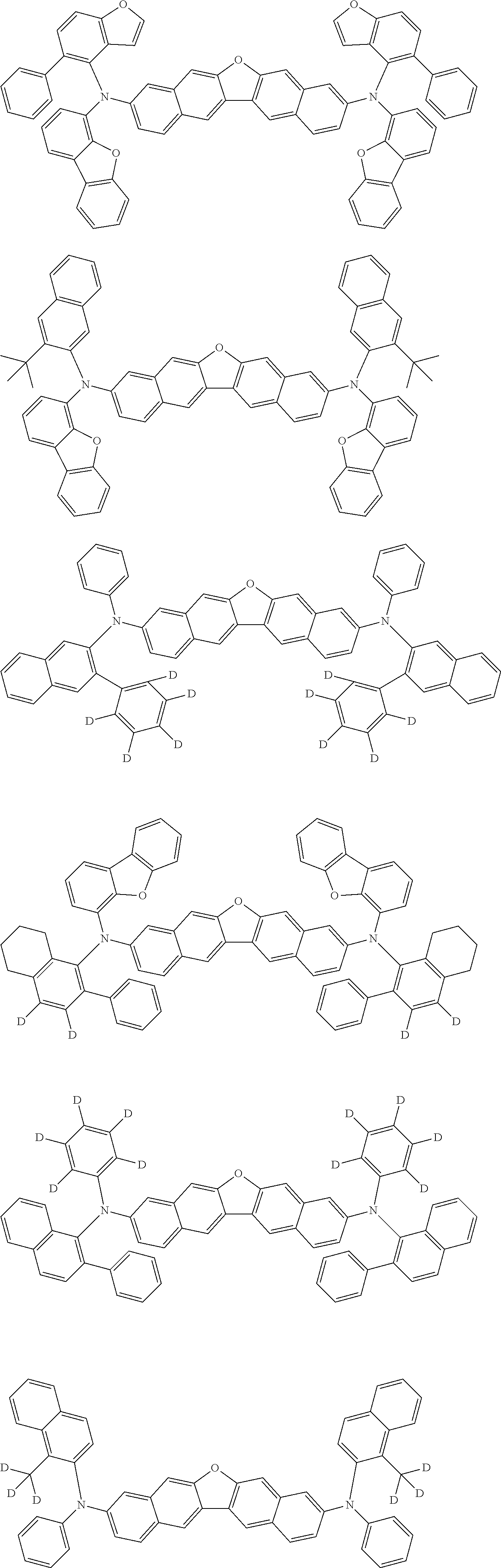

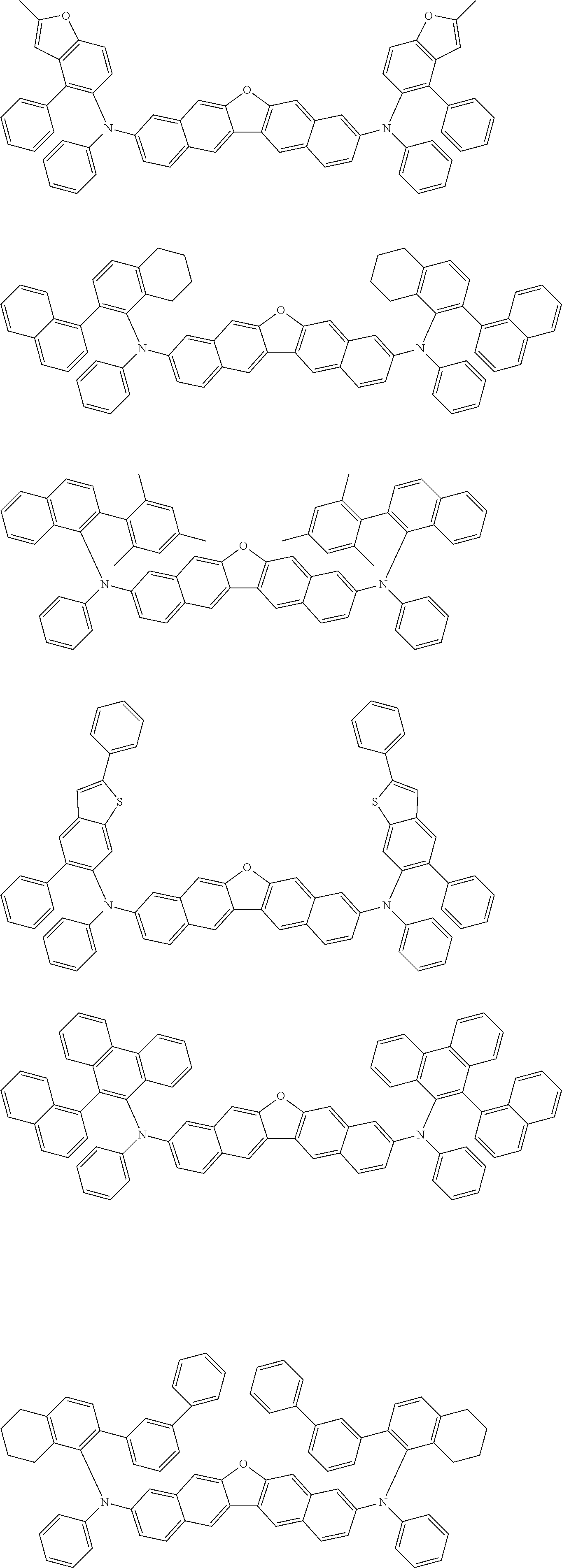

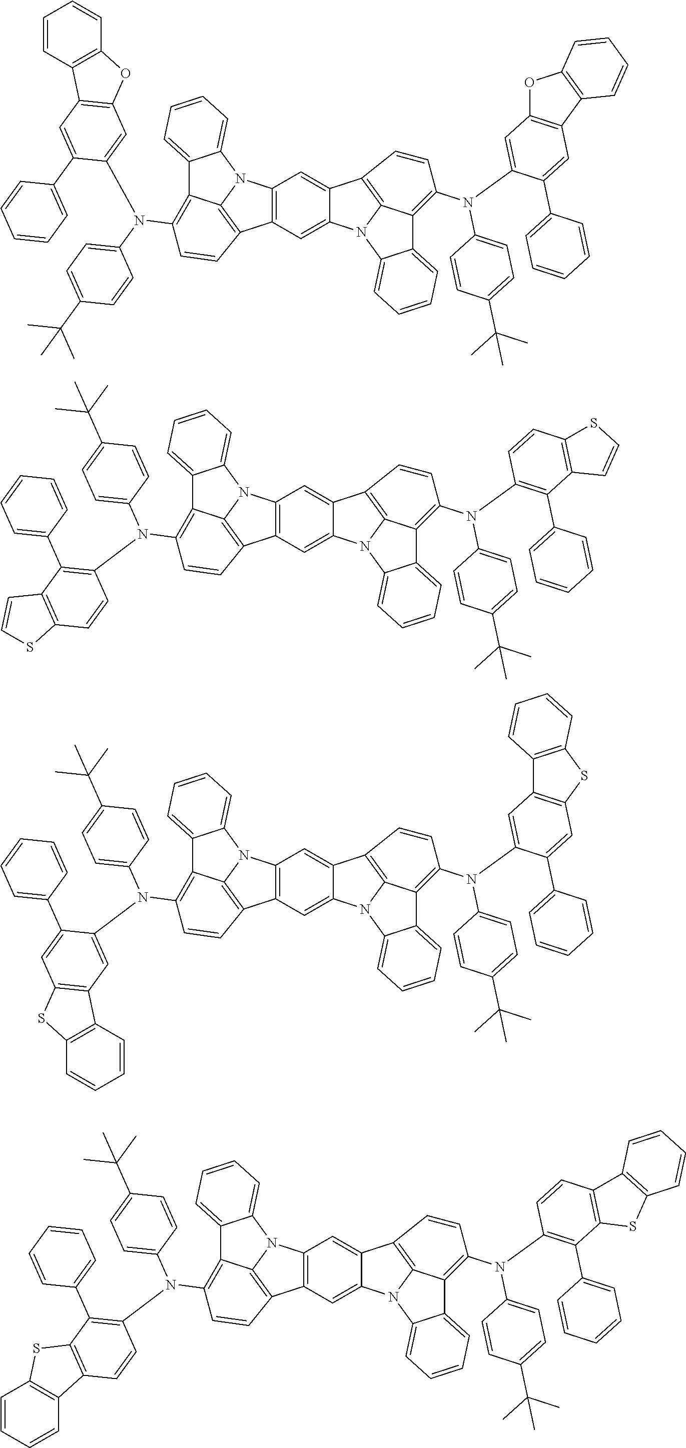

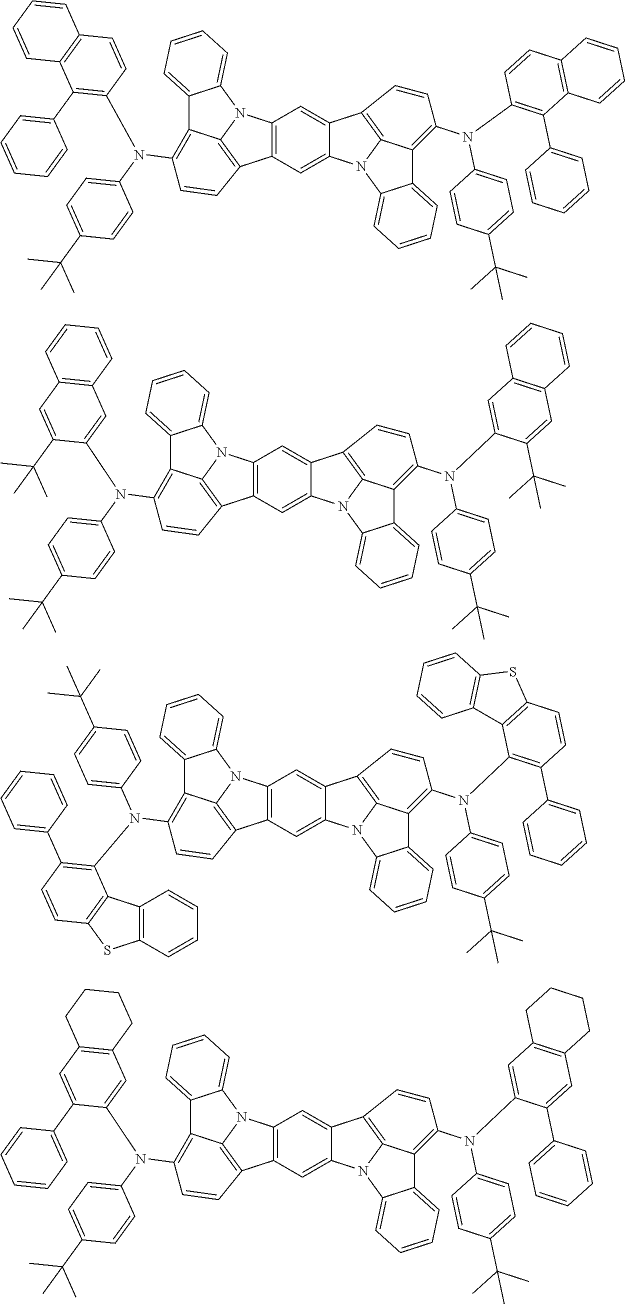

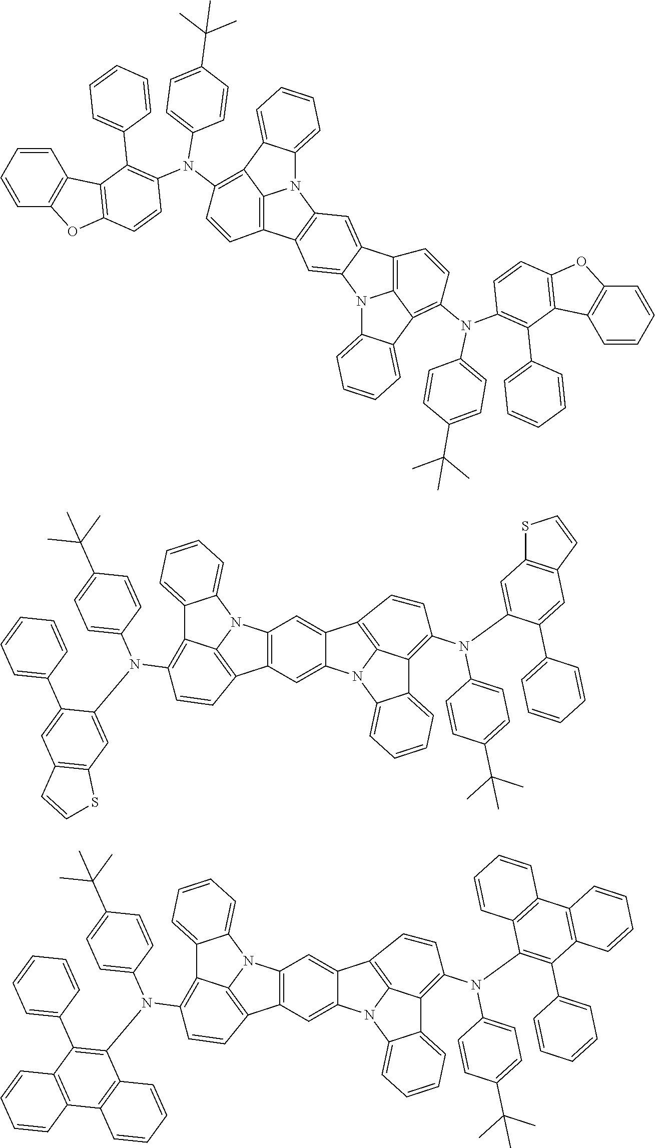

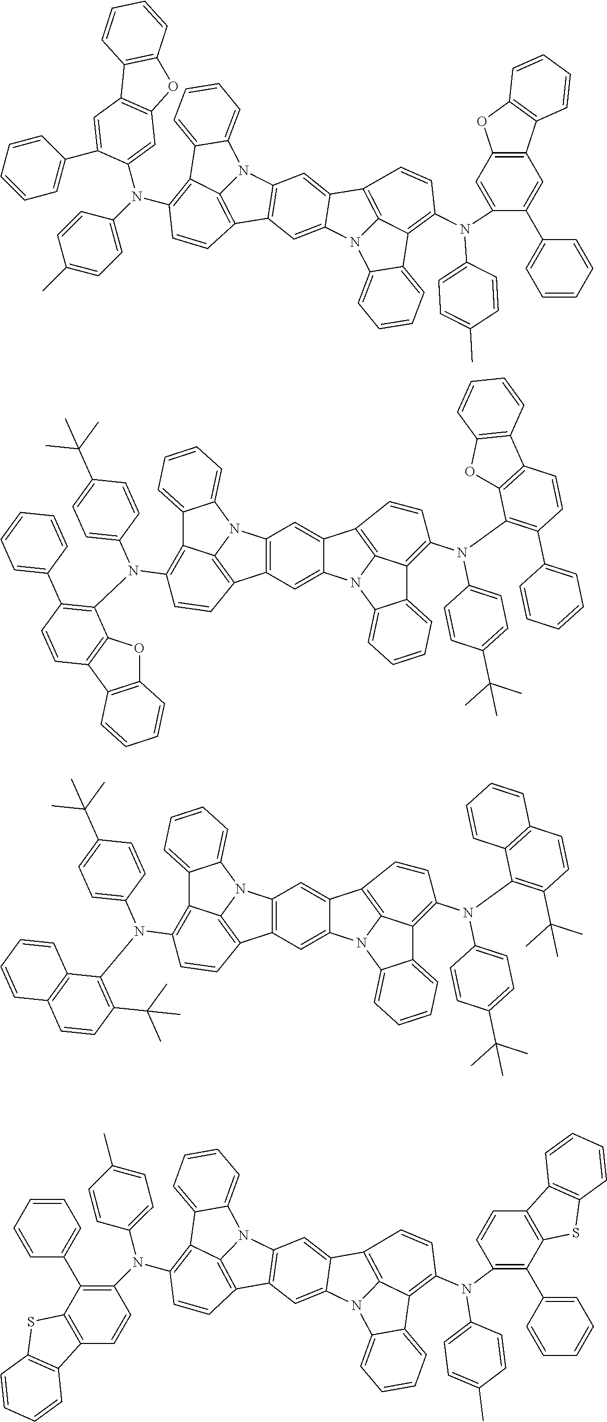

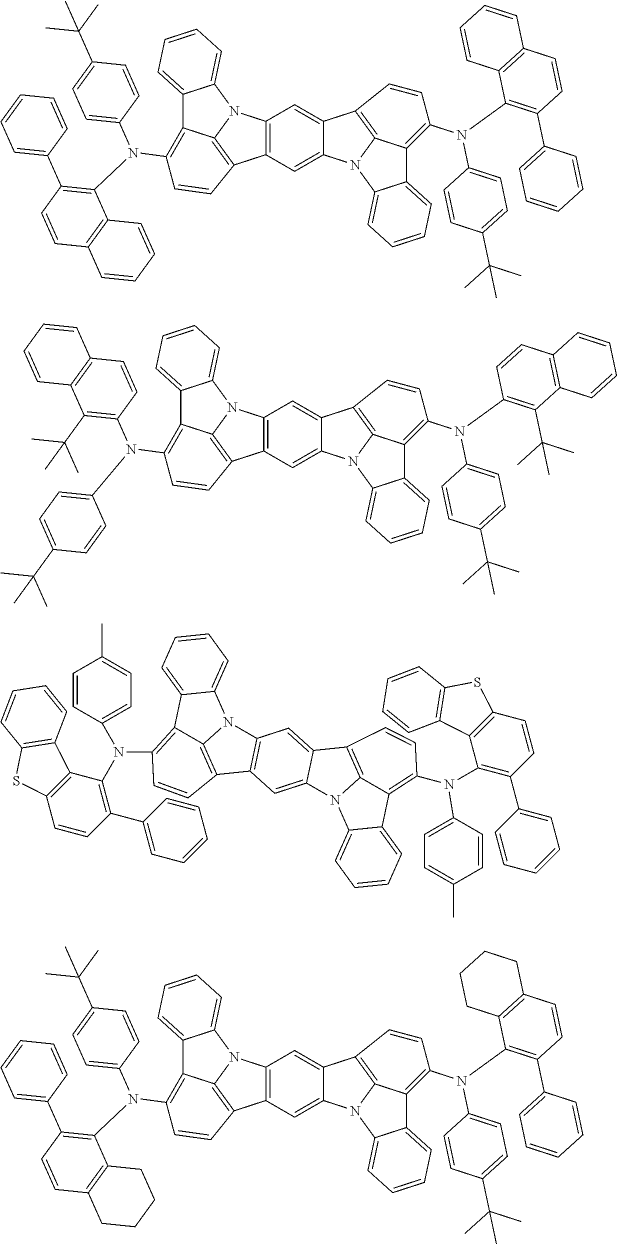

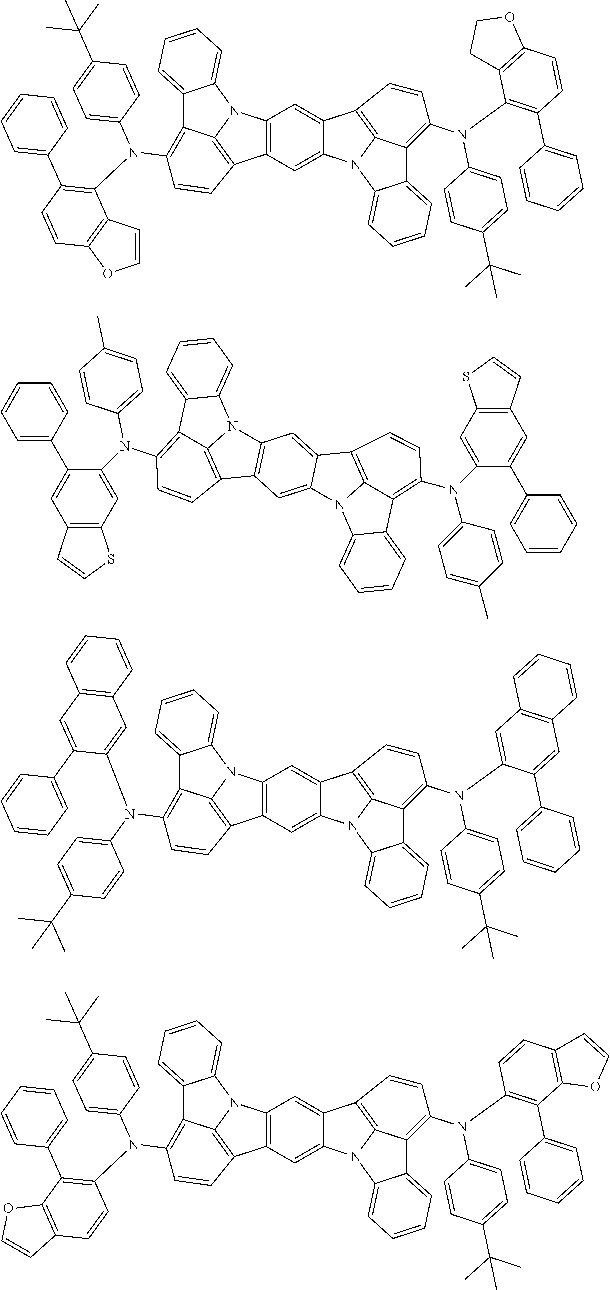

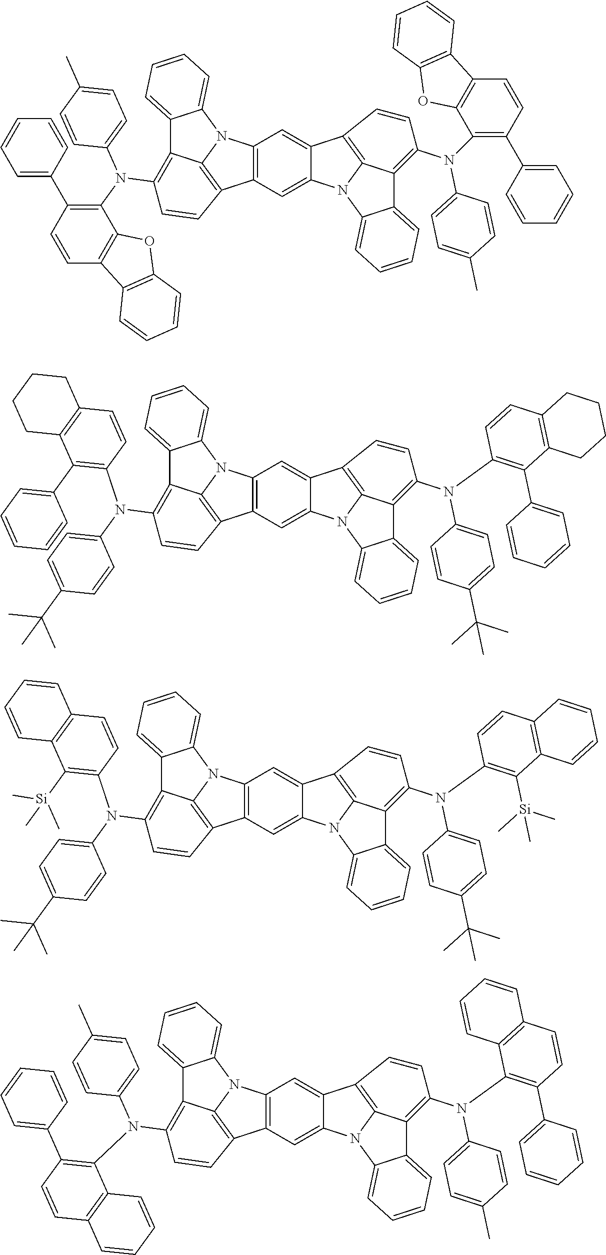

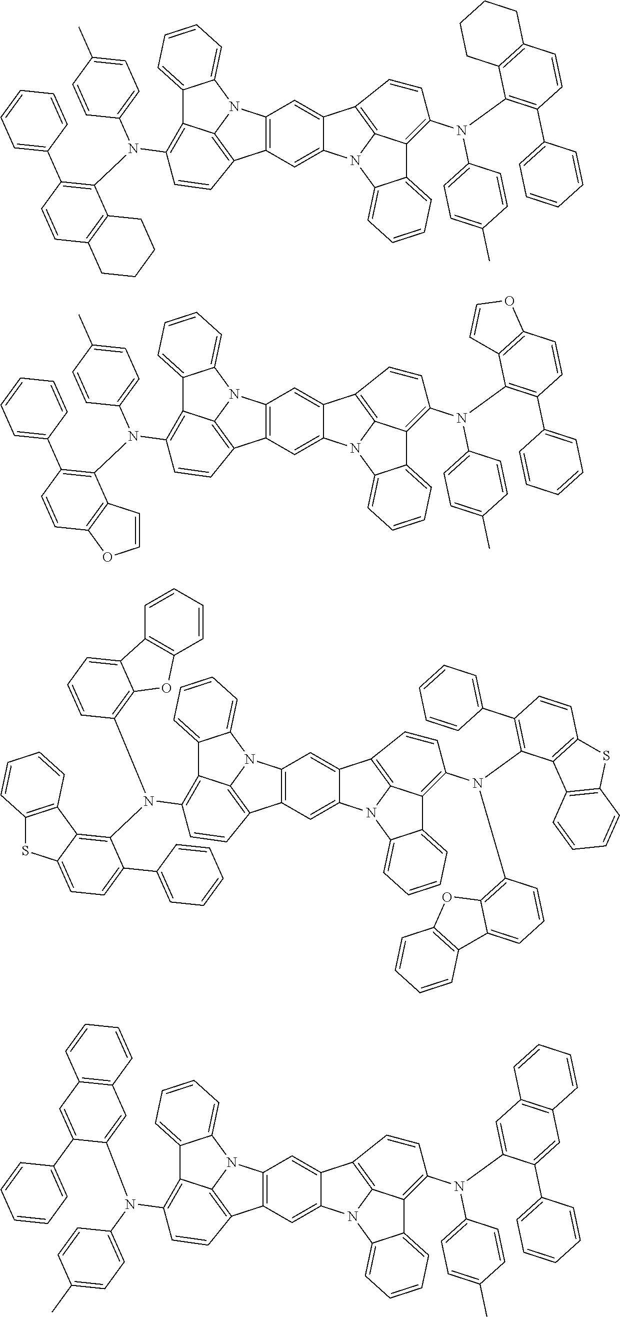

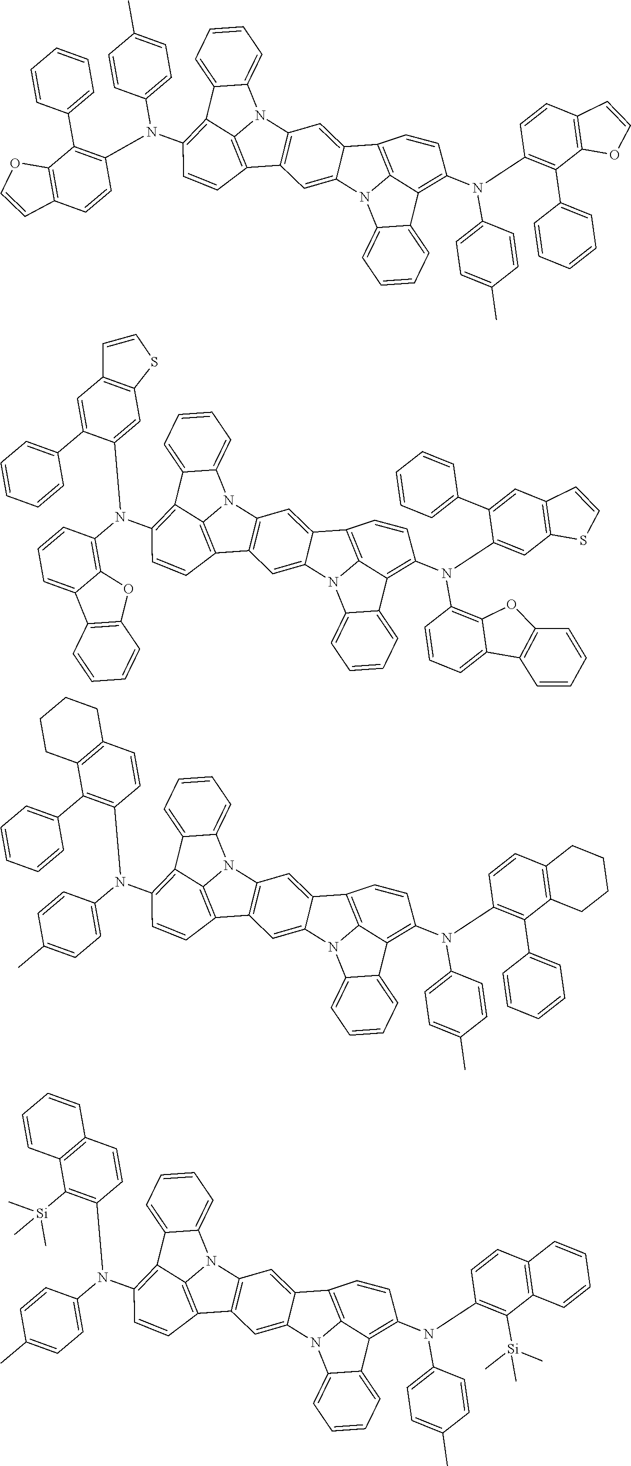

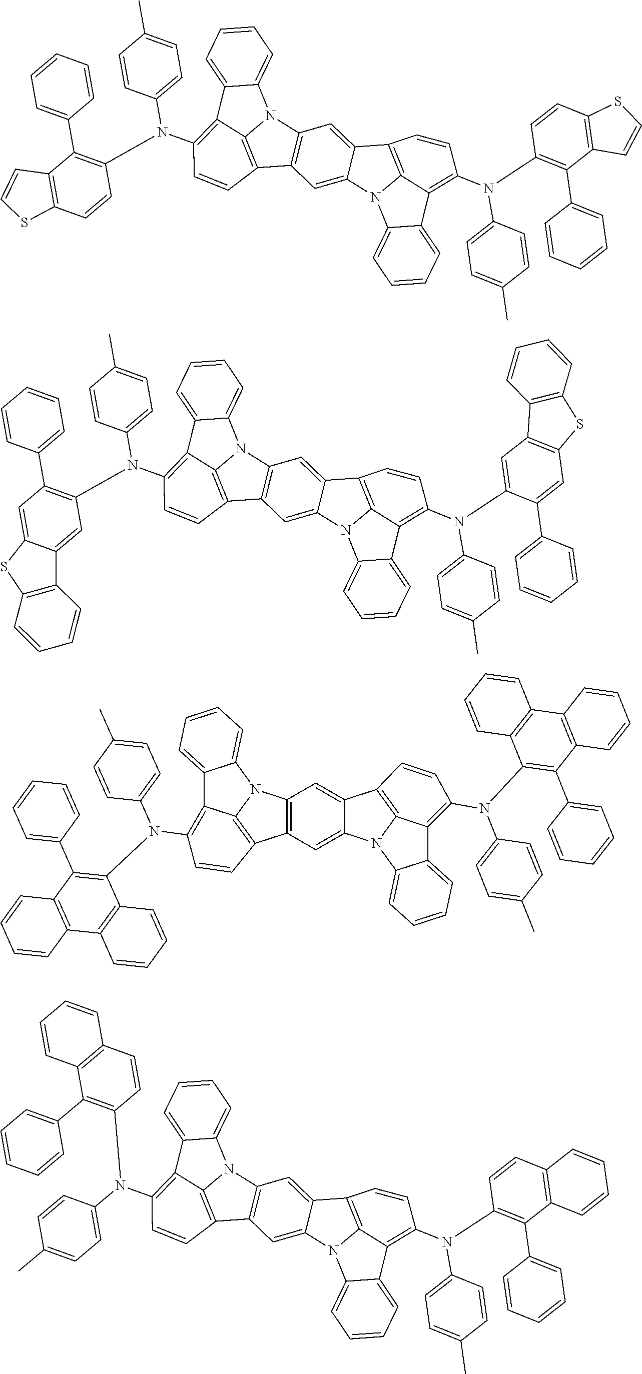

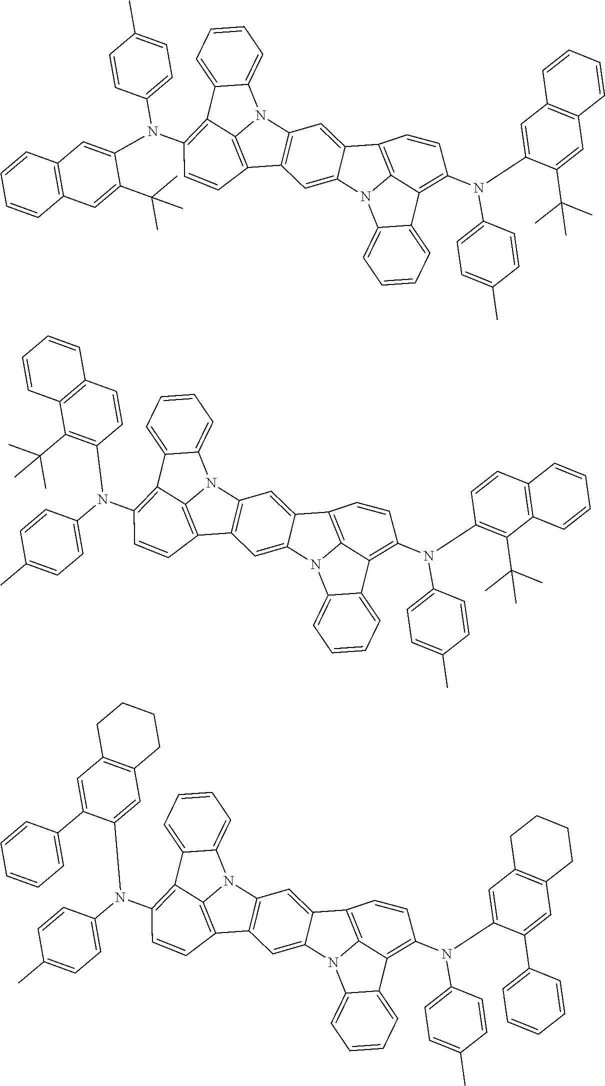 [SMILES: Cc1ccc(N(c2cc3ccccc3cc2C(C)(C)C)c2ccc3c4cc5c(cc4n4c6ccccc6c2c34)c2ccc(N(c3ccc(C)cc3)c3cc4ccccc4cc3C(C)(C)C)c3c4ccccc4n5c23)cc1 ; Cc1ccc(N(c2ccc3ccccc3c2C(C)(C)C)c2ccc3c4cc5c(cc4n4c6ccccc6c2c34)c2ccc(N(c3ccc(C)cc3)c3ccc4ccccc4c3C(C)(C)C)c3c4ccccc4n5c23)cc1 ; Cc1ccc(N(c2cc3c(cc2-c2ccccc2)CCCC3)c2ccc3c4cc5c(cc4n4c6ccccc6c2c34)c2ccc(N(c3ccc(C)cc3)c3cc4c(cc3-c3ccccc3)CCCC4)c3c4ccccc4n5c23)cc1]

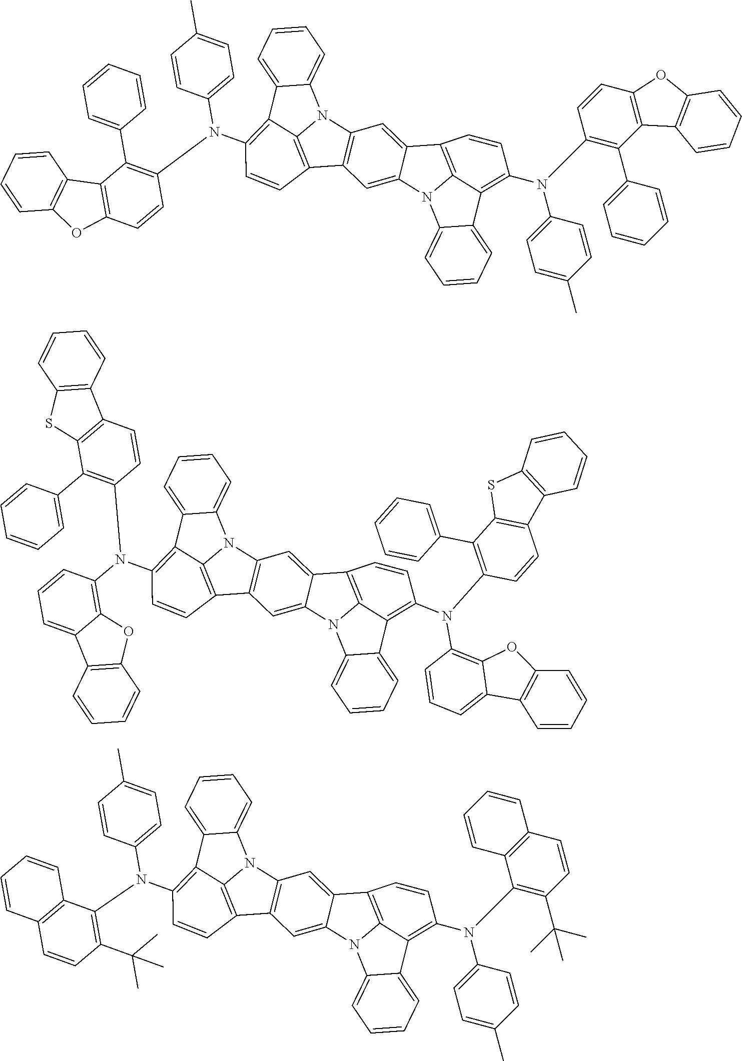

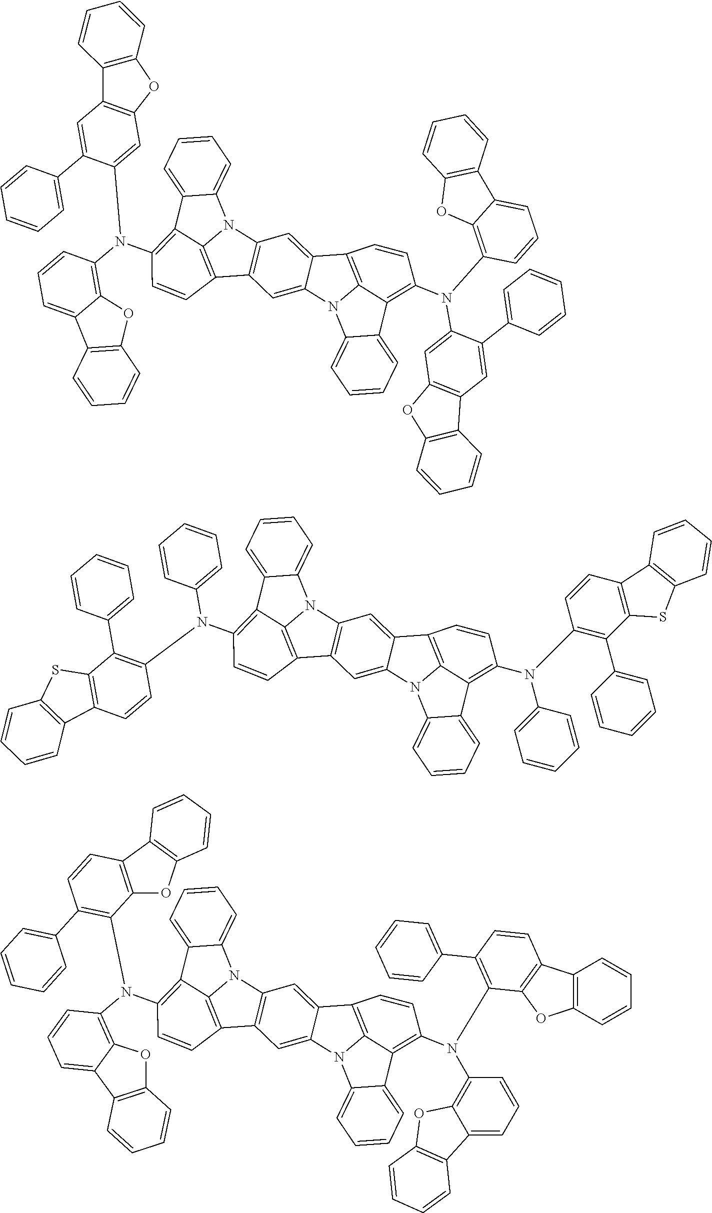 [SMILES: c1ccc(-c2cc3c(cc2N(c2cccc4c2oc2ccccc24)c2ccc4c5cc6c(cc5n5c7ccccc7c2c45)c2ccc(N(c4cc5oc7ccccc7c5cc4-c4ccccc4)c4cccc5c4oc4ccccc45)c4c5ccccc5n6c24)oc2ccccc23)cc1 ; c1ccc(-c2c(N(c3ccccc3)c3ccc4c5cc6c(cc5n5c7ccccc7c3c45)c3ccc(N(c4ccccc4)c4ccc5c(sc7ccccc75)c4-c4ccccc4)c4c5ccccc5n6c34)ccc3c2sc2ccccc23)cc1 ; c1ccc(-c2ccc3c(oc4ccccc43)c2N(c2cccc3c2oc2ccccc23)c2ccc3c4cc5c(cc4n4c6ccccc6c2c34)c2ccc(N(c3cccc4c3oc3ccccc34)c3c(-c4ccccc4)ccc4c3oc3ccccc34)c3c4ccccc4n5c23)cc1]

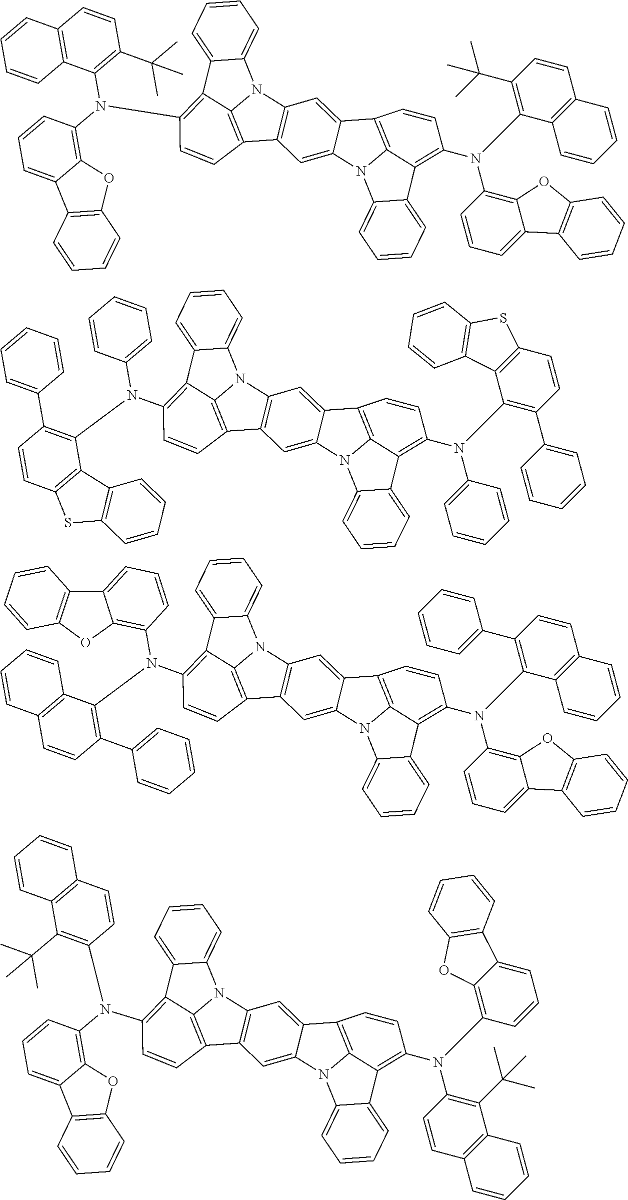

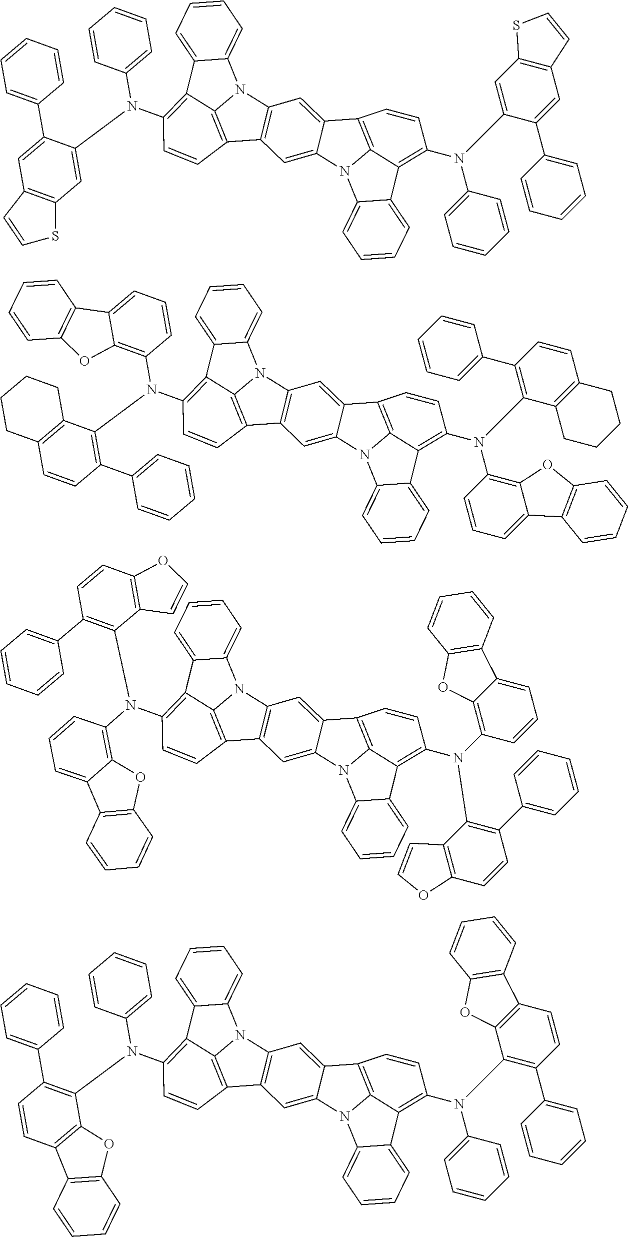

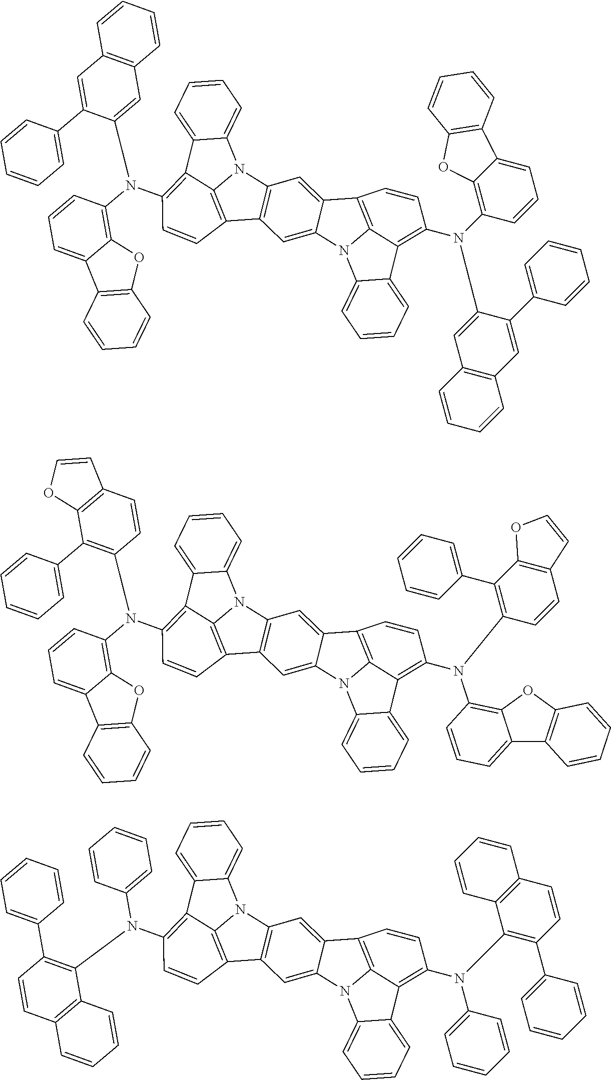

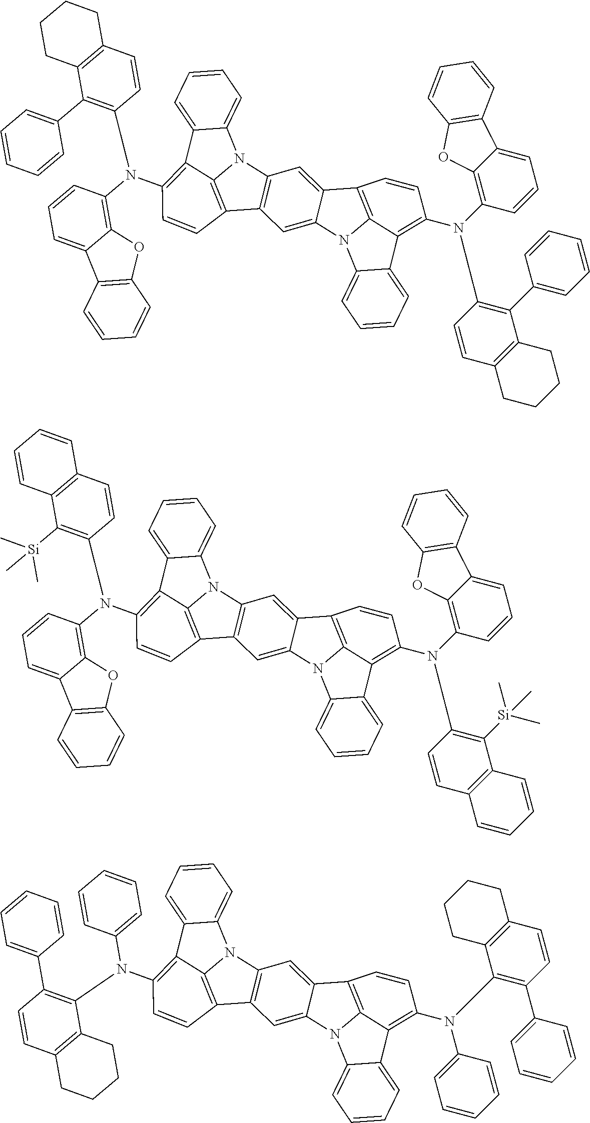

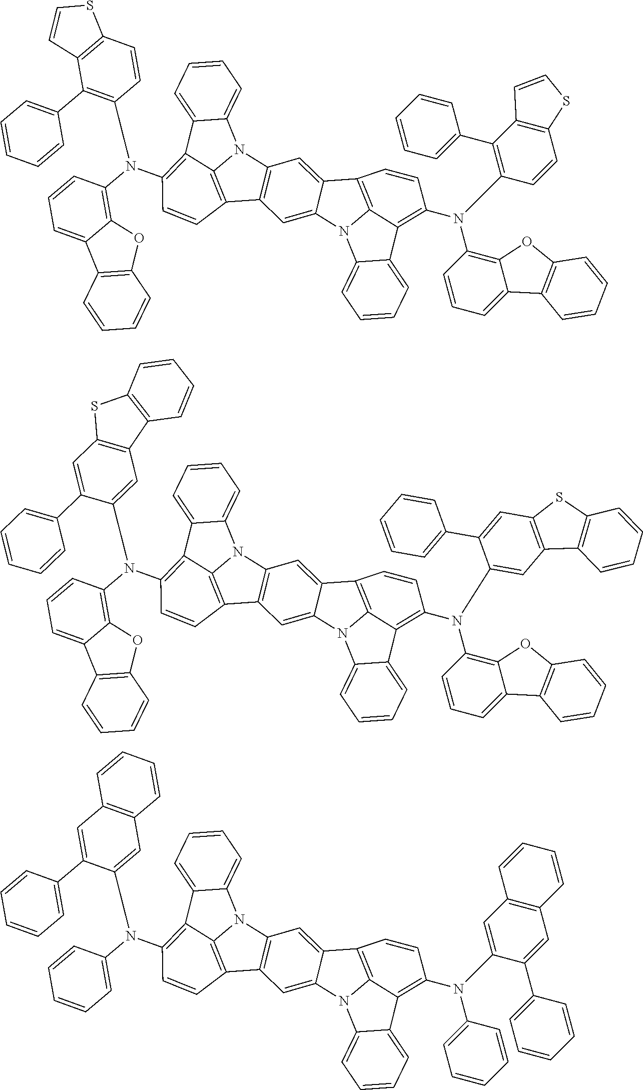

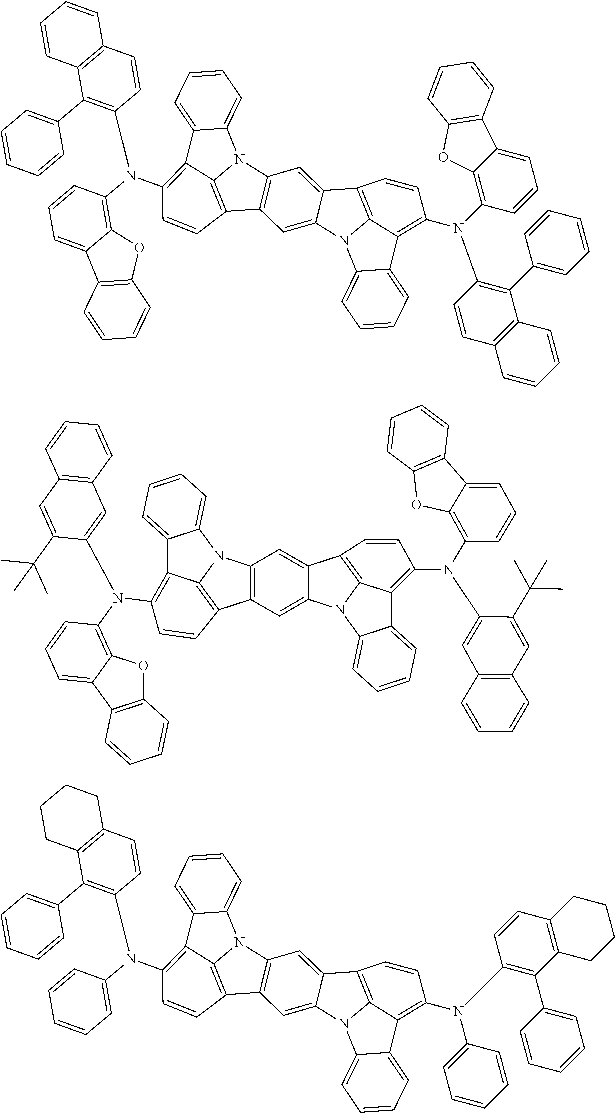

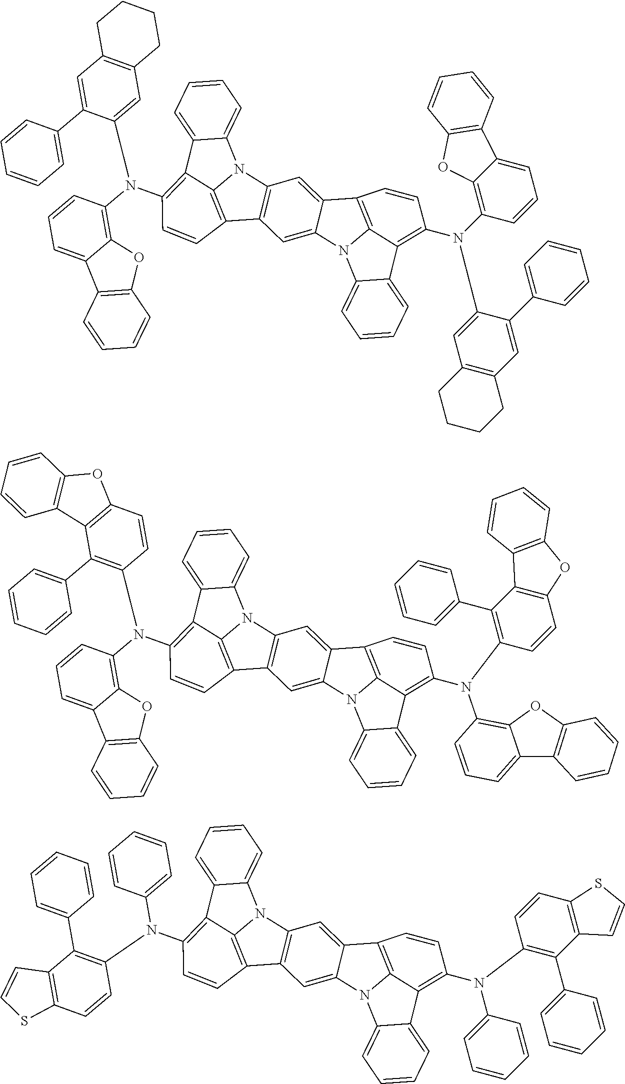

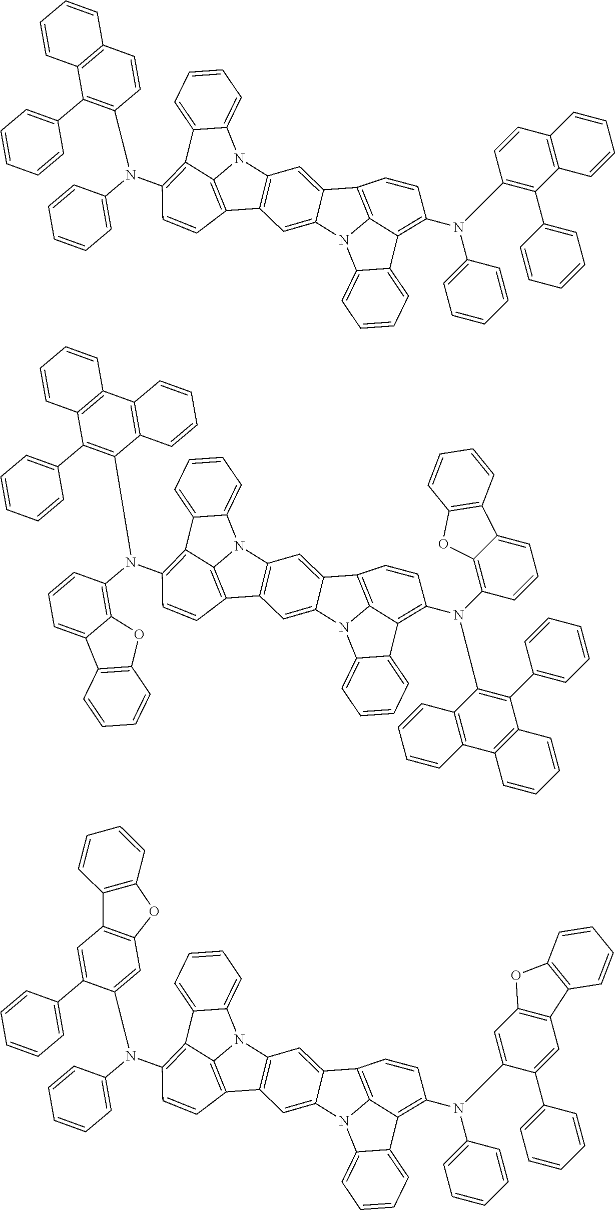 [SMILES: c1ccc(-c2c(N(c3ccccc3)c3ccc4c5cc6c(cc5n5c7ccccc7c3c45)c3ccc(N(c4ccccc4)c4ccc5ccccc5c4-c4ccccc4)c4c5ccccc5n6c34)ccc3ccccc23)cc1 ; c1ccc(-c2c(N(c3cccc4c3oc3ccccc34)c3ccc4c5cc6c(cc5n5c7ccccc7c3c45)c3ccc(N(c4c(-c5ccccc5)c5ccccc5c5ccccc45)c4cccc5c4oc4ccccc45)c4c5ccccc5n6c34)c3ccccc3c3ccccc23)cc1 ; c1ccc(-c2cc3c(cc2N(c2ccccc2)c2ccc4c5cc6c(cc5n5c7ccccc7c2c45)c2ccc(N(c4ccccc4)c4cc5oc7ccccc7c5cc4-c4ccccc4)c4c5ccccc5n6c24)oc2ccccc23)cc1]

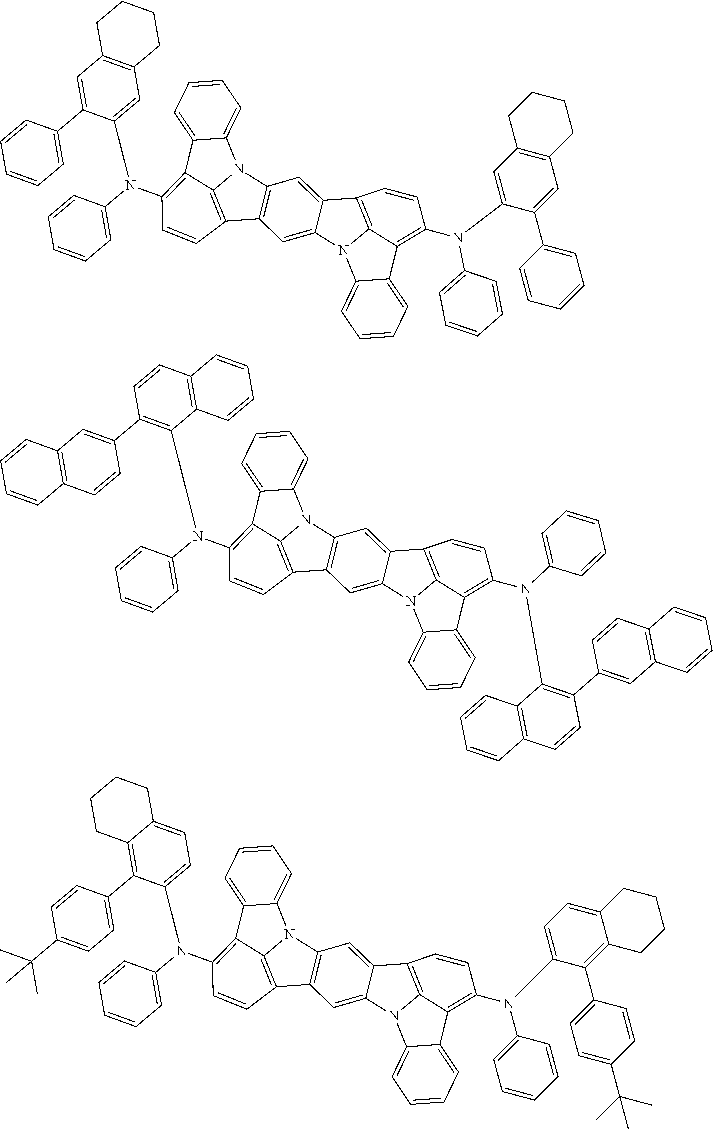 [SMILES: c1ccc(-c2cc3c(cc2N(c2ccccc2)c2ccc4c5cc6c(cc5n5c7ccccc7c2c45)c2ccc(N(c4ccccc4)c4cc5c(cc4-c4ccccc4)CCCC5)c4c5ccccc5n6c24)CCCC3)cc1 ; c1ccc(N(c2c(-c3ccc4ccccc4c3)ccc3ccccc23)c2ccc3c4cc5c(cc4n4c6ccccc6c2c34)c2ccc(N(c3ccccc3)c3c(-c4ccc6ccccc6c4)ccc4ccccc34)c3c4ccccc4n5c23)cc1 ; CC(C)(C)c1ccc(-c2c(N(c3ccccc3)c3ccc4c5cc6c(cc5n5c7ccccc7c3c45)c3ccc(N(c4ccccc4)c4ccc5c(c4-c4ccc(C(C)(C)C)cc4)CCCC5)c4c5ccccc5n6c34)ccc3c2CCCC3)cc1]

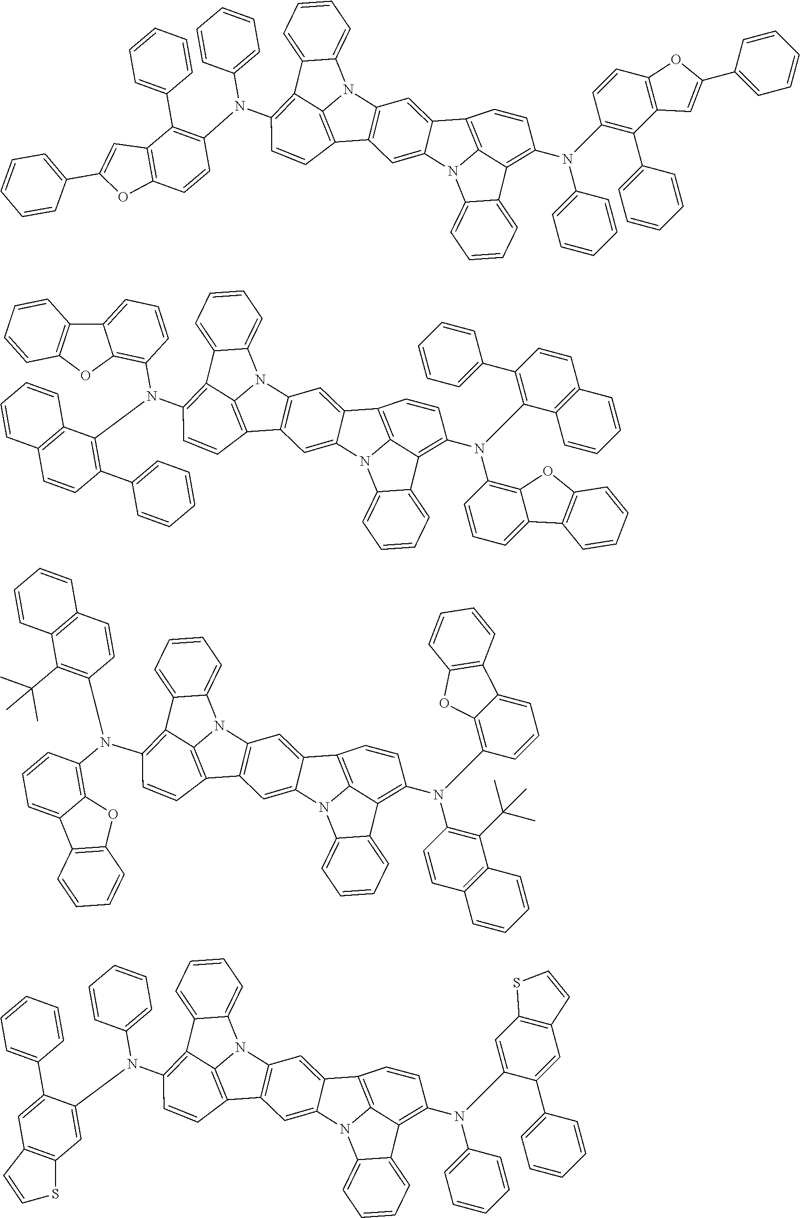

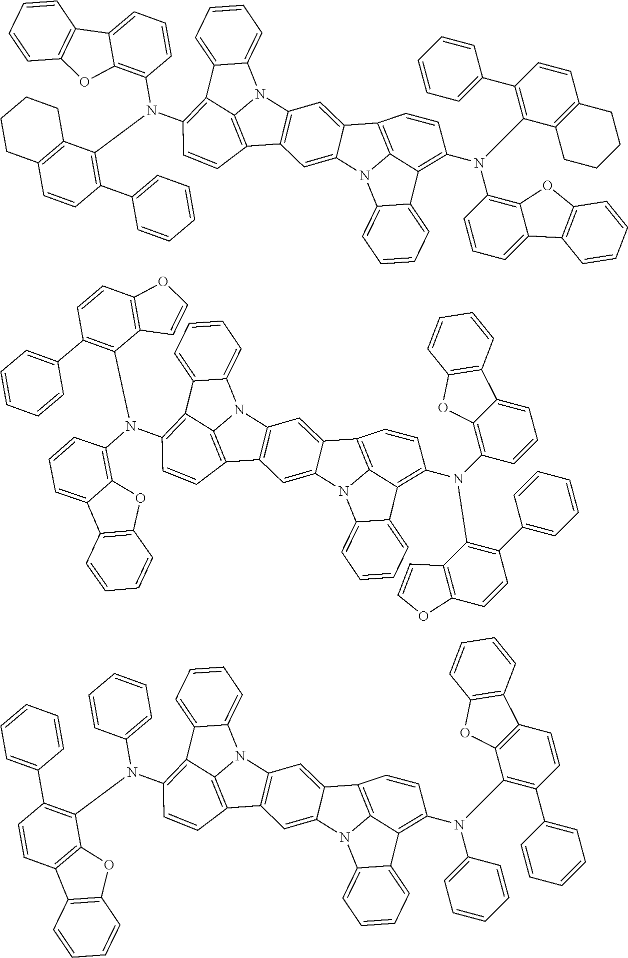

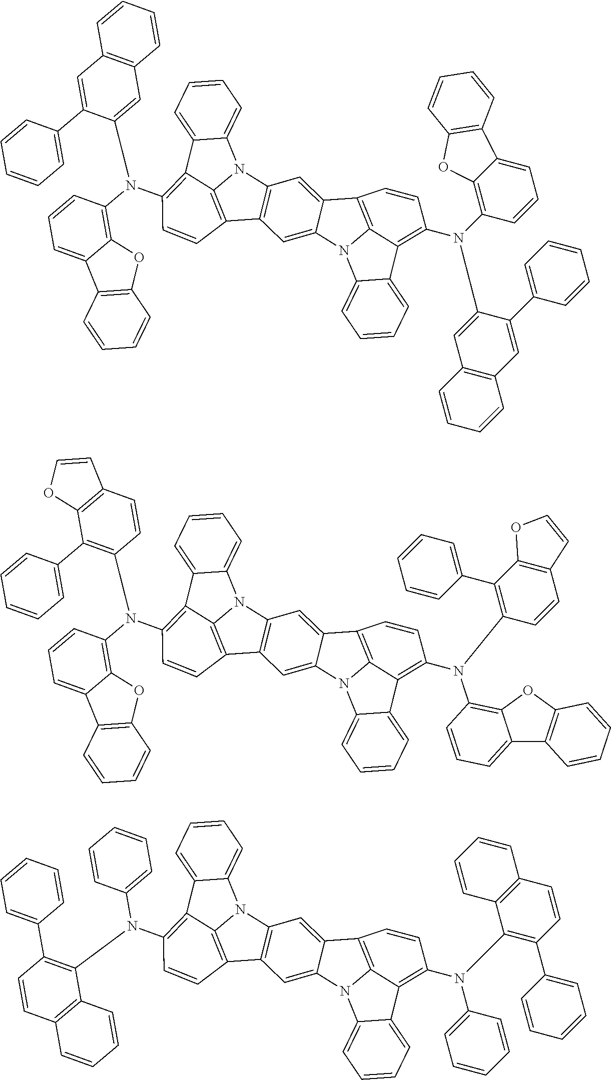

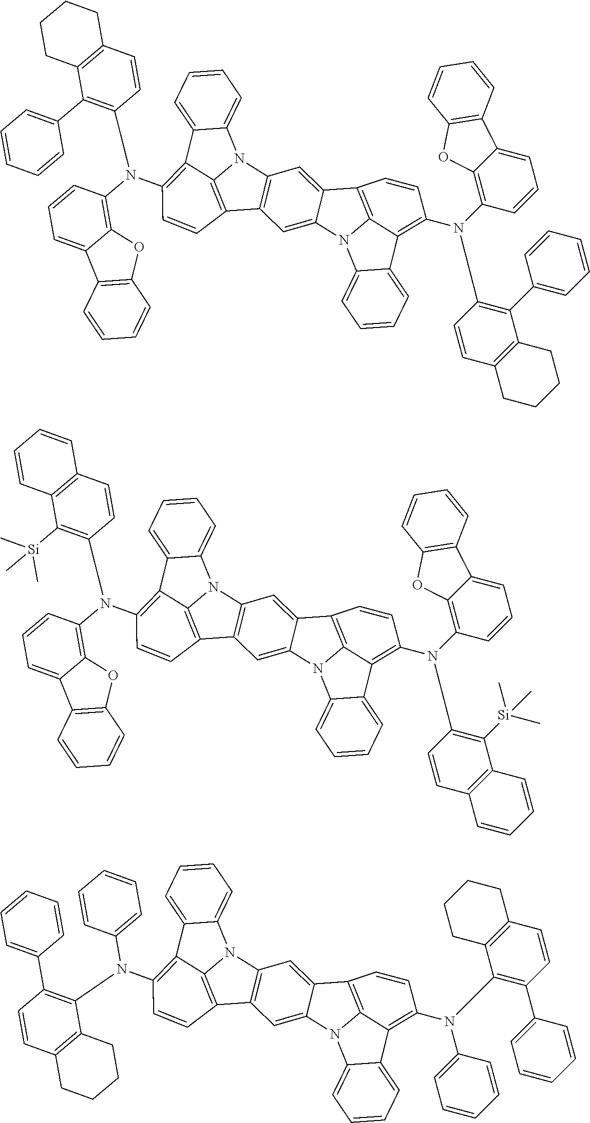

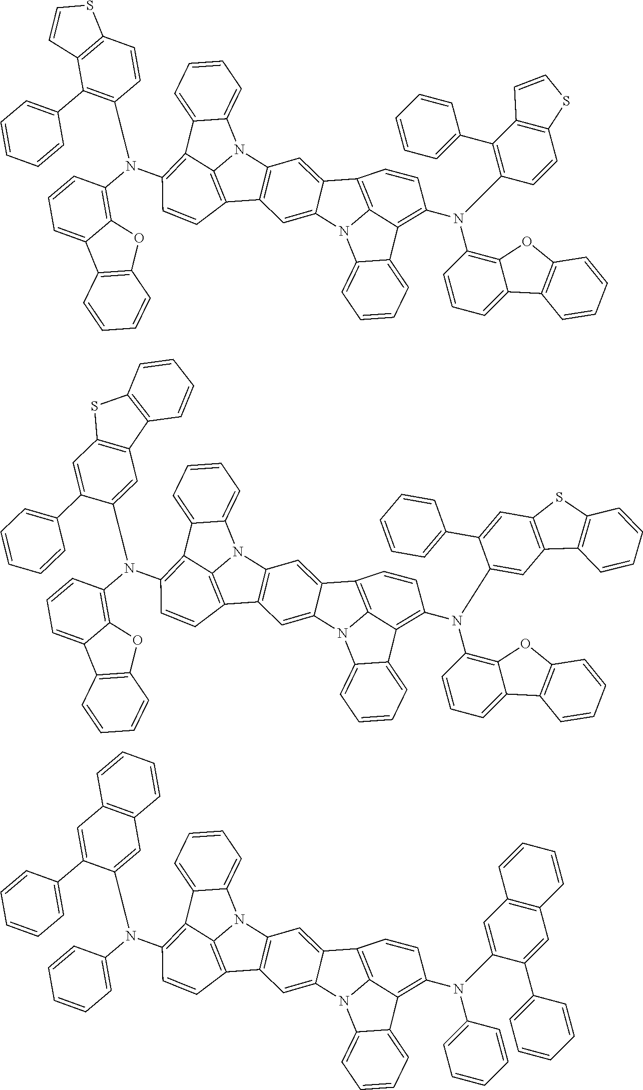

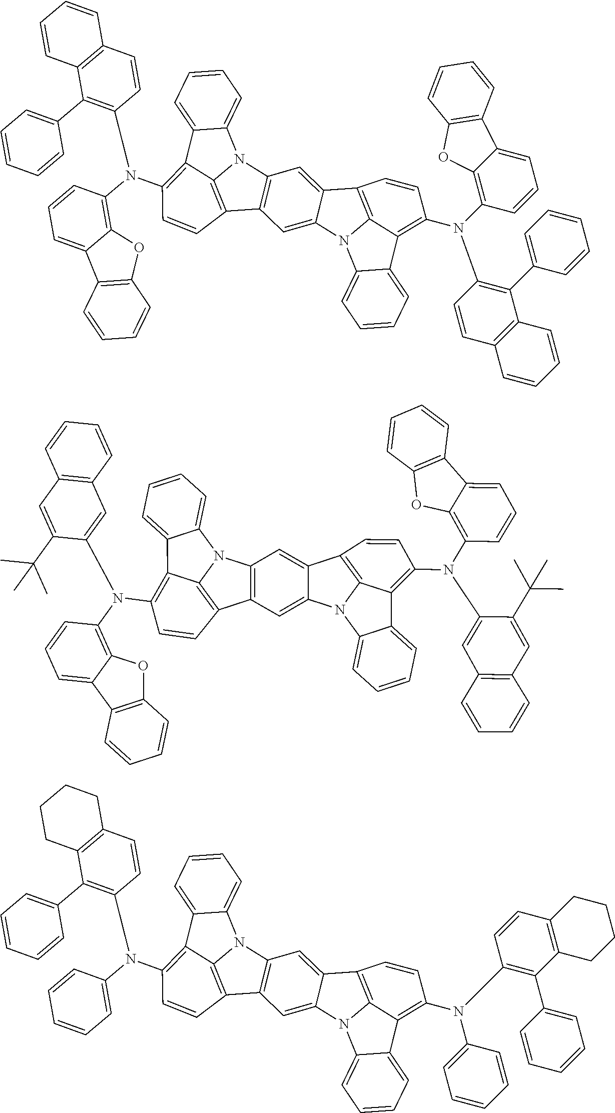

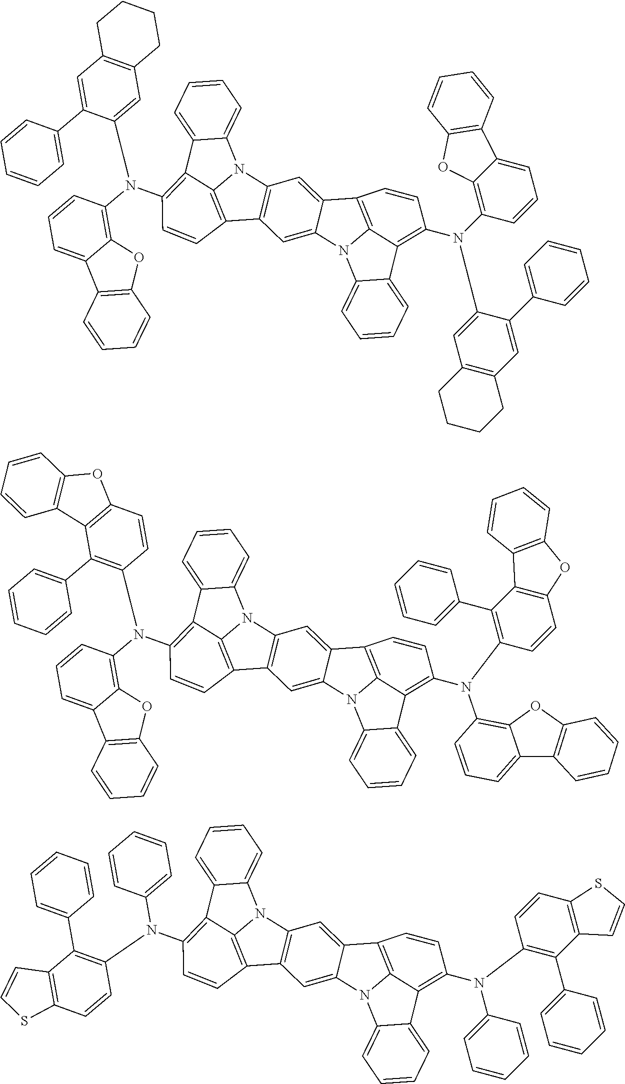

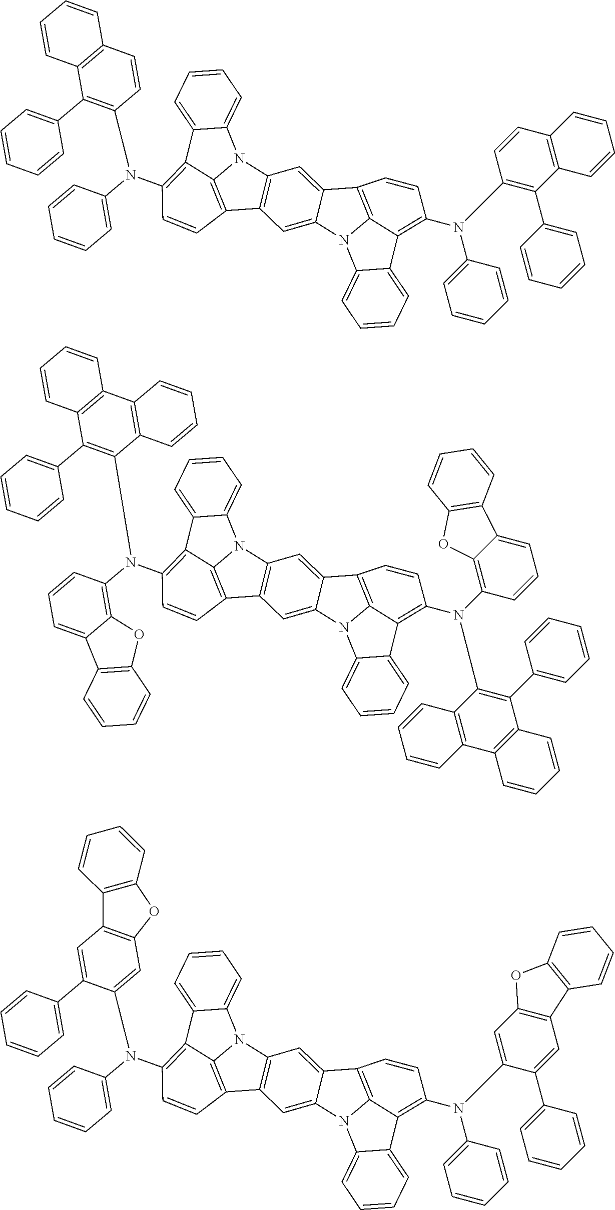 [SMILES: c1ccc(-c2c(N(c3ccccc3)c3ccc4c5cc6c(cc5n5c7ccccc7c3c45)c3ccc(N(c4ccccc4)c4ccc5ccccc5c4-c4ccccc4)c4c5ccccc5n6c34)ccc3ccccc23)cc1 ; c1ccc(-c2c(N(c3cccc4c3oc3ccccc34)c3ccc4c5cc6c(cc5n5c7ccccc7c3c45)c3ccc(N(c4c(-c5ccccc5)c5ccccc5c5ccccc45)c4cccc5c4oc4ccccc45)c4c5ccccc5n6c34)c3ccccc3c3ccccc23)cc1 ; c1ccc(-c2cc3c(cc2N(c2ccccc2)c2ccc4c5cc6c(cc5n5c7ccccc7c2c45)c2ccc(N(c4ccccc4)c4cc5oc7ccccc7c5cc4-c4ccccc4)c4c5ccccc5n6c24)oc2ccccc23)cc1]

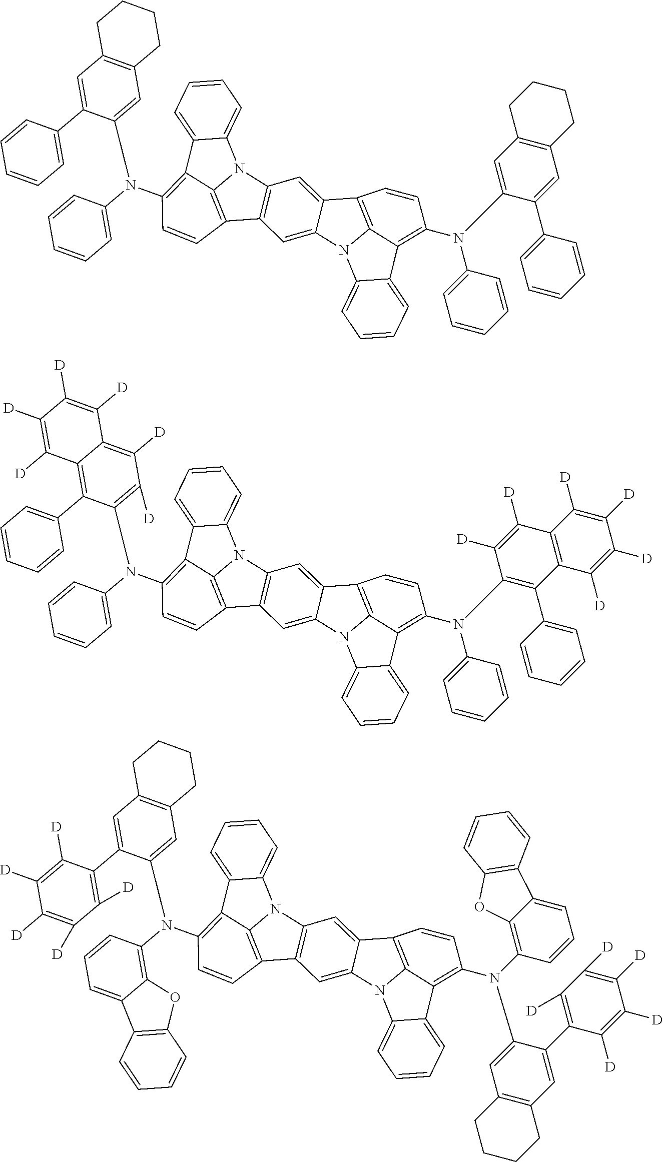

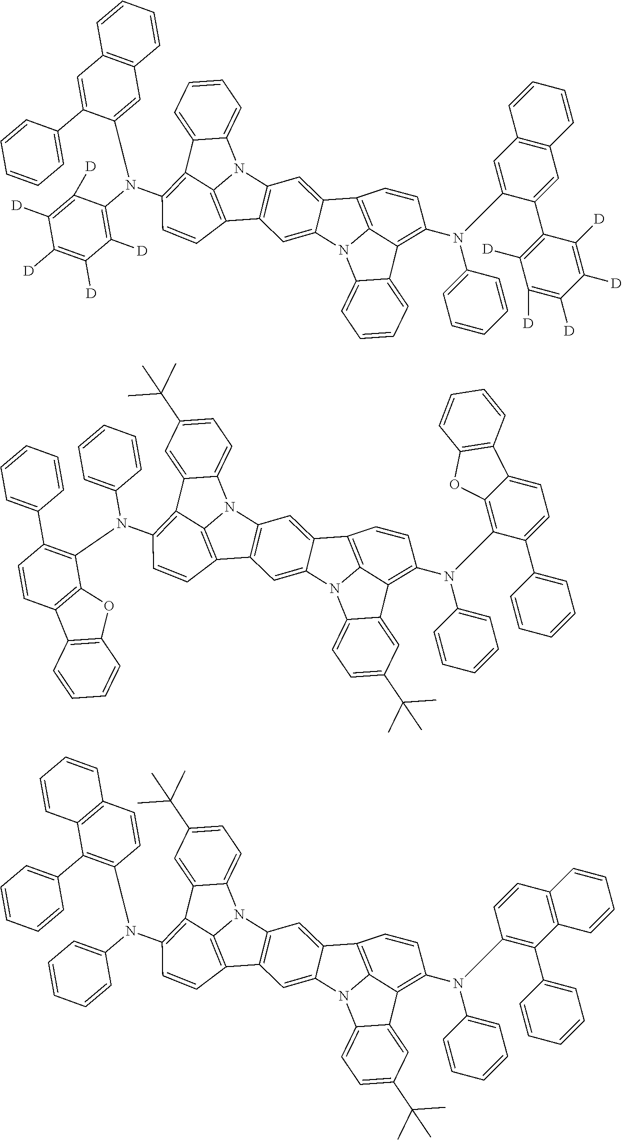 [SMILES: [2H]c1c([2H])c([2H])c(-c2cc3ccccc3cc2N(c2ccccc2)c2ccc3c4cc5c(cc4n4c6ccccc6c2c34)c2ccc(N(c3cc4ccccc4cc3-c3ccccc3)c3c([2H])c([2H])c([2H])c([2H])c3[2H])c3c4ccccc4n5c23)c([2H])c1[2H] ; CC(C)(C)c1ccc2c(c1)c1c(N(c3ccccc3)c3c(-c4ccccc4)ccc4c3oc3ccccc34)ccc3c4cc5c(cc4n2c31)c1ccc(N(c2ccccc2)c2c(-c3ccccc3)ccc3c2oc2ccccc23)c2c3cc(C(C)(C)C)ccc3n5c12 ; CC(C)(C)c1ccc2c(c1)c1c(N(c3ccccc3)c3ccc4ccccc4c3-c3ccccc3)ccc3c4cc5c(cc4n2c31)c1ccc(N(c2ccccc2)c2ccc3ccccc3c2-c2ccccc2)c2c3cc(C(C)(C)C)ccc3n5c12]

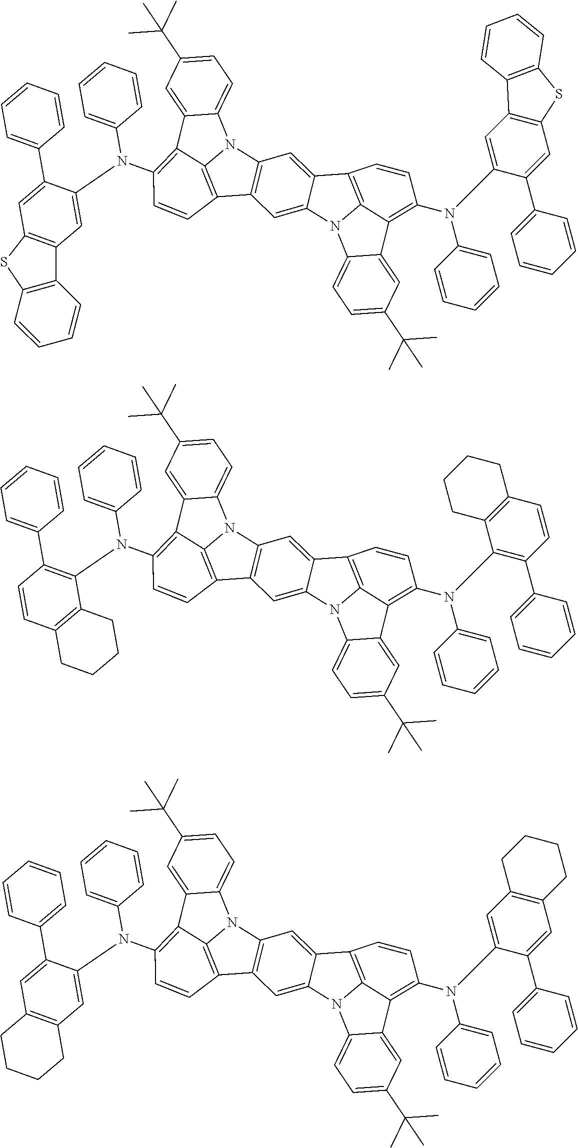 [SMILES: CC(C)(C)c1ccc2c(c1)c1c(N(c3ccccc3)c3cc4c(cc3-c3ccccc3)sc3ccccc34)ccc3c4cc5c(cc4n2c31)c1ccc(N(c2ccccc2)c2cc3c(cc2-c2ccccc2)sc2ccccc23)c2c3cc(C(C)(C)C)ccc3n5c12 ; CC(C)(C)c1ccc2c(c1)c1c(N(c3ccccc3)c3c(-c4ccccc4)ccc4c3CCCC4)ccc3c4cc5c(cc4n2c31)c1ccc(N(c2ccccc2)c2c(-c3ccccc3)ccc3c2CCCC3)c2c3cc(C(C)(C)C)ccc3n5c12 ; CC(C)(C)c1ccc2c(c1)c1c(N(c3ccccc3)c3cc4c(cc3-c3ccccc3)CCCC4)ccc3c4cc5c(cc4n2c31)c1ccc(N(c2ccccc2)c2cc3c(cc2-c2ccccc2)CCCC3)c2c3cc(C(C)(C)C)ccc3n5c12]

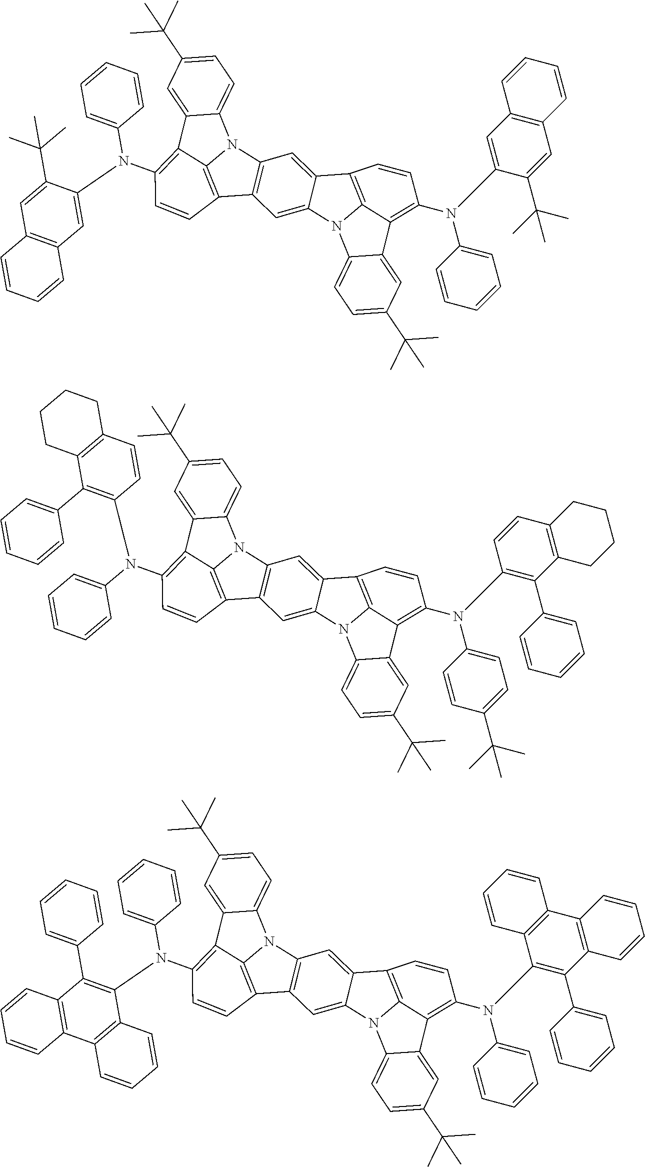 [SMILES: CC(C)(C)c1ccc2c(c1)c1c(N(c3ccccc3)c3cc4ccccc4cc3C(C)(C)C)ccc3c4cc5c(cc4n2c31)c1ccc(N(c2ccccc2)c2cc3ccccc3cc2C(C)(C)C)c2c3cc(C(C)(C)C)ccc3n5c12 ; CC(C)(C)c1ccc(N(c2ccc3c(c2-c2ccccc2)CCCC3)c2ccc3c4cc5c(cc4n4c6ccc(C(C)(C)C)cc6c2c34)c2ccc(N(c3ccccc3)c3ccc4c(c3-c3ccccc3)CCCC4)c3c4cc(C(C)(C)C)ccc4n5c23)cc1 ; CC(C)(C)c1ccc2c(c1)c1c(N(c3ccccc3)c3c(-c4ccccc4)c4ccccc4c4ccccc34)ccc3c4cc5c(cc4n2c31)c1ccc(N(c2ccccc2)c2c(-c3ccccc3)c3ccccc3c3ccccc23)c2c3cc(C(C)(C)C)ccc3n5c12]

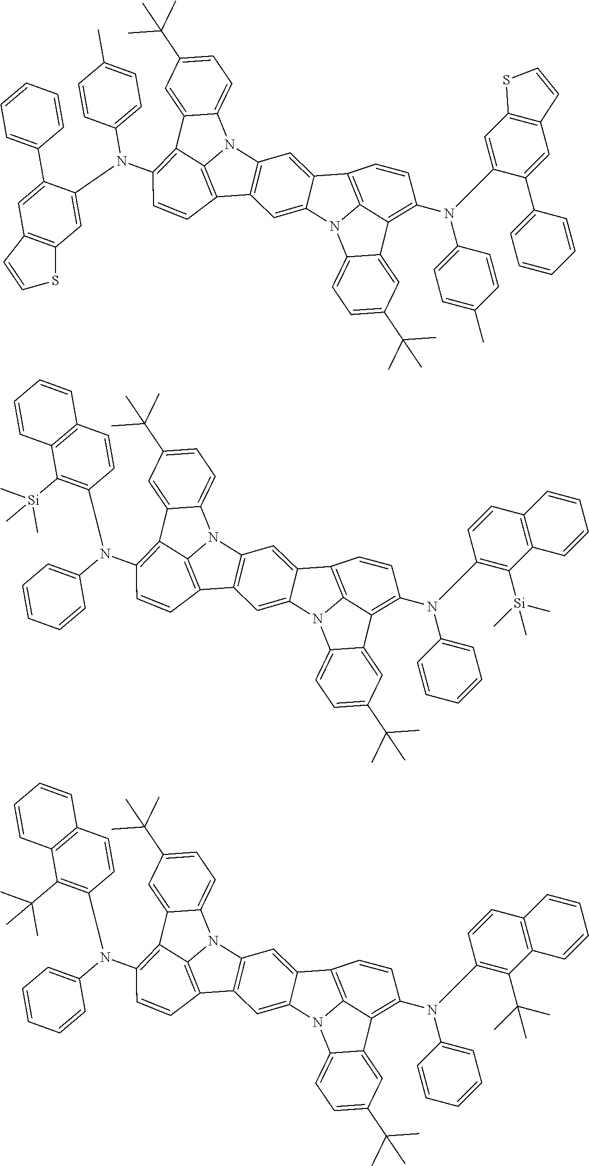 [SMILES: Cc1ccc(N(c2cc3sccc3cc2-c2ccccc2)c2ccc3c4cc5c(cc4n4c6ccc(C(C)(C)C)cc6c2c34)c2ccc(N(c3ccc(C)cc3)c3cc4sccc4cc3-c3ccccc3)c3c4cc(C(C)(C)C)ccc4n5c23)cc1 ; CC(C)(C)c1ccc2c(c1)c1c(N(c3ccccc3)c3ccc4ccccc4c3[Si](C)(C)C)ccc3c4cc5c(cc4n2c31)c1ccc(N(c2ccccc2)c2ccc3ccccc3c2[Si](C)(C)C)c2c3cc(C(C)(C)C)ccc3n5c12 ; CC(C)(C)c1ccc2c(c1)c1c(N(c3ccccc3)c3ccc4ccccc4c3C(C)(C)C)ccc3c4cc5c(cc4n2c31)c1ccc(N(c2ccccc2)c2ccc3ccccc3c2C(C)(C)C)c2c3cc(C(C)(C)C)ccc3n5c12]

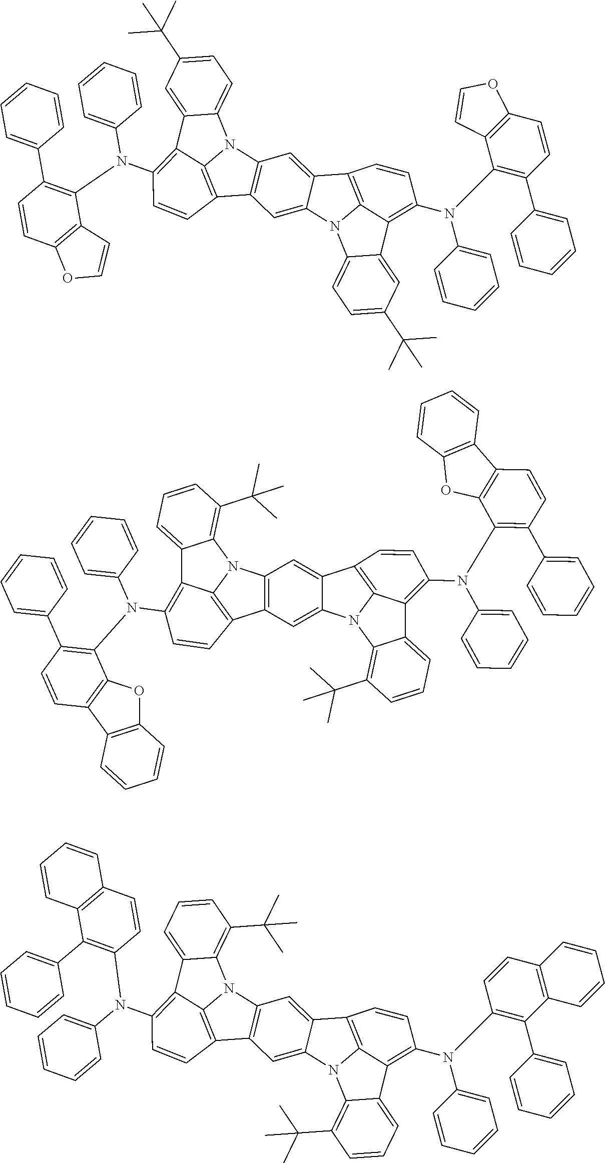

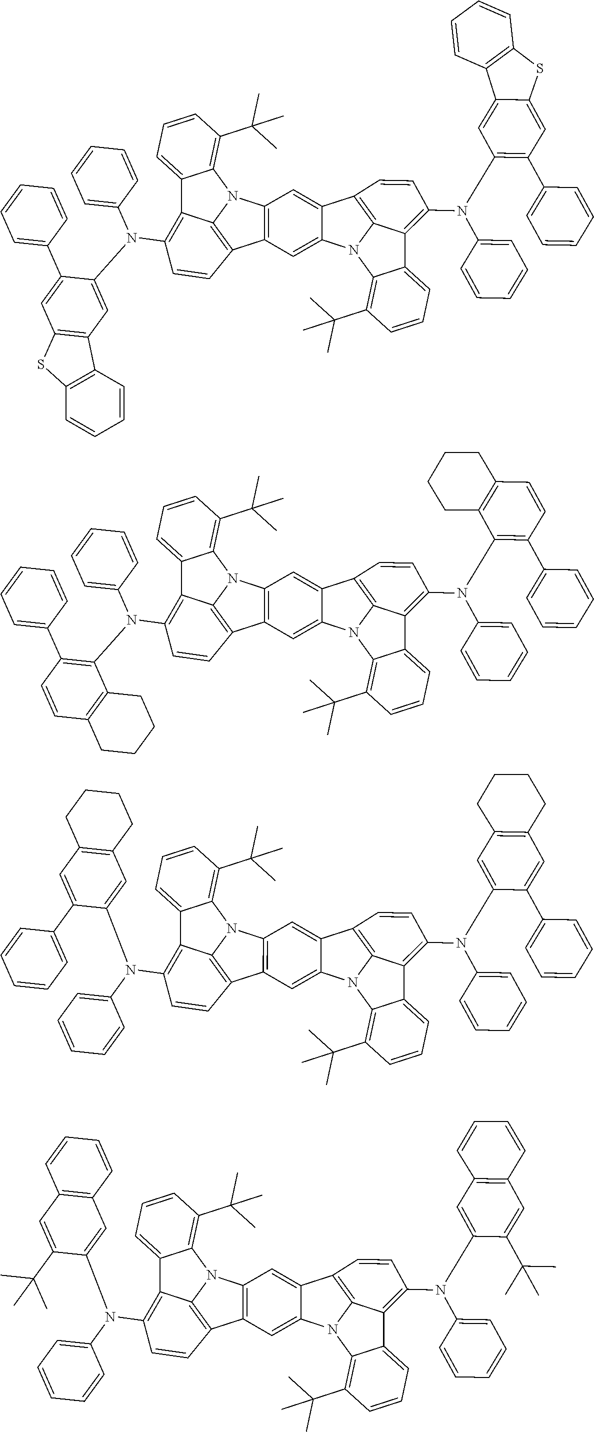

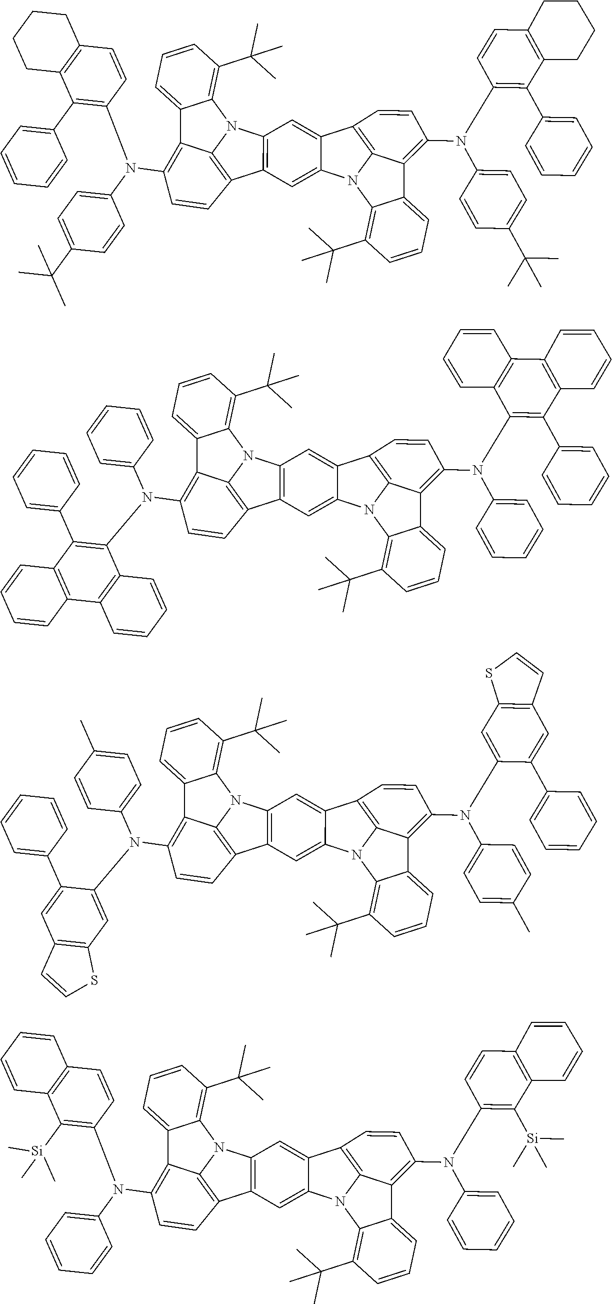

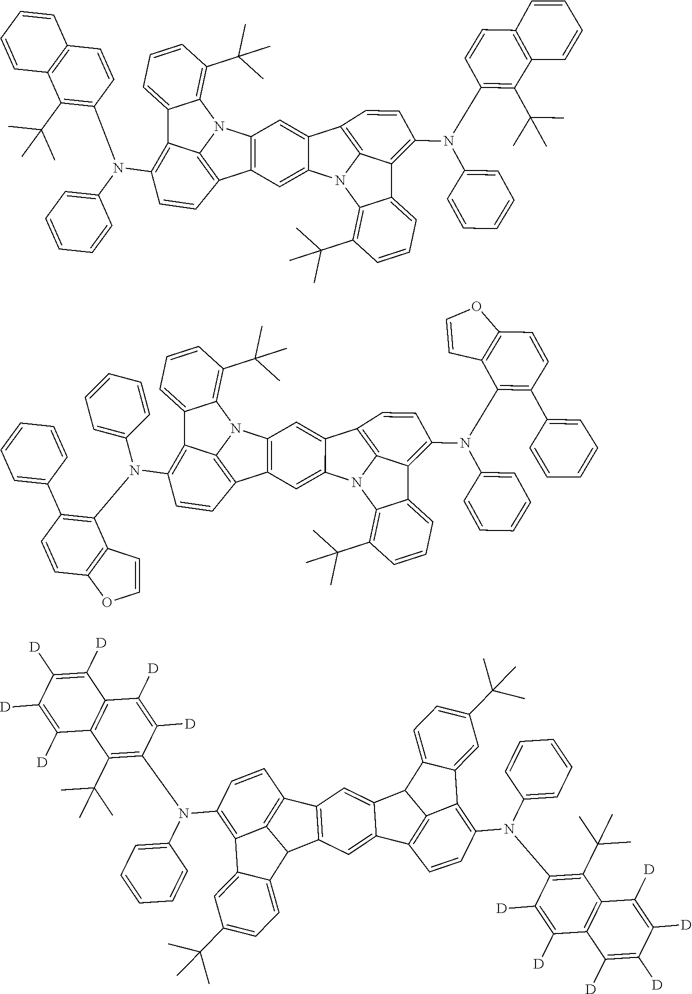

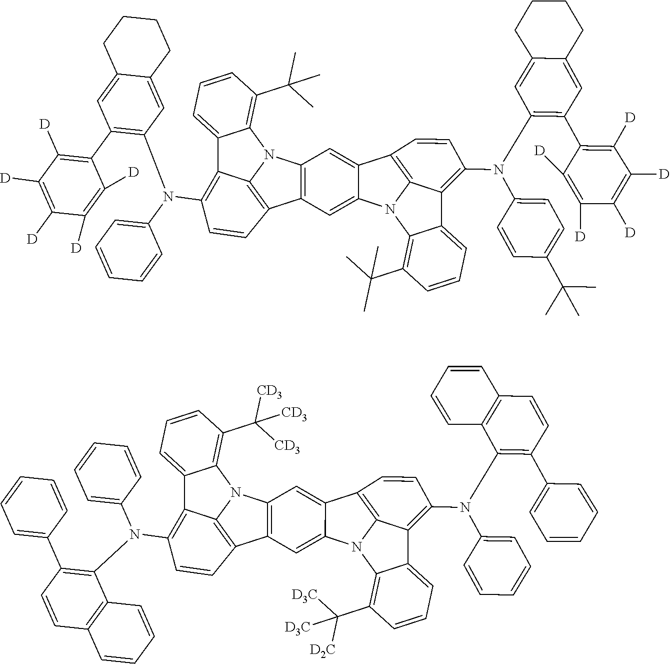

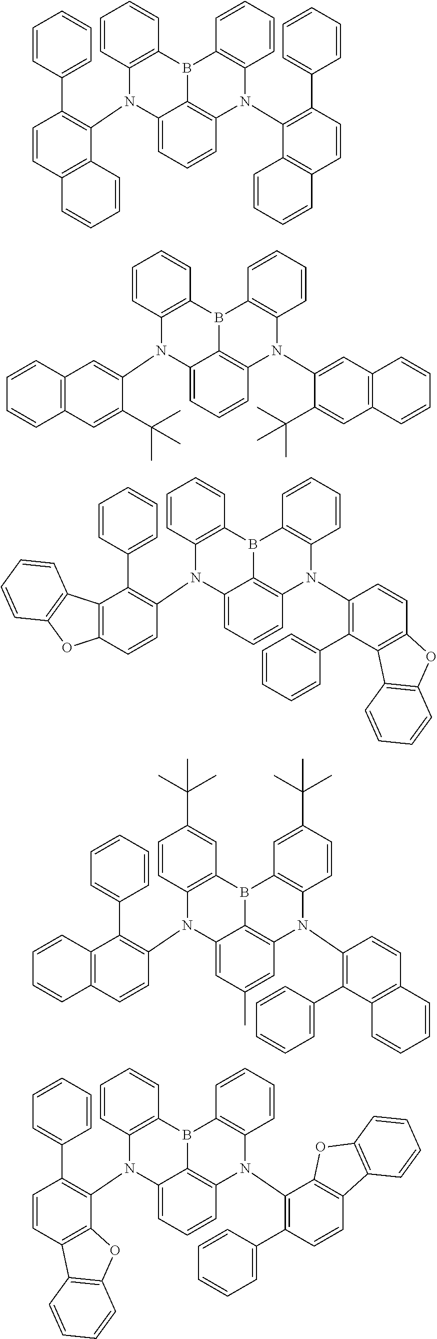

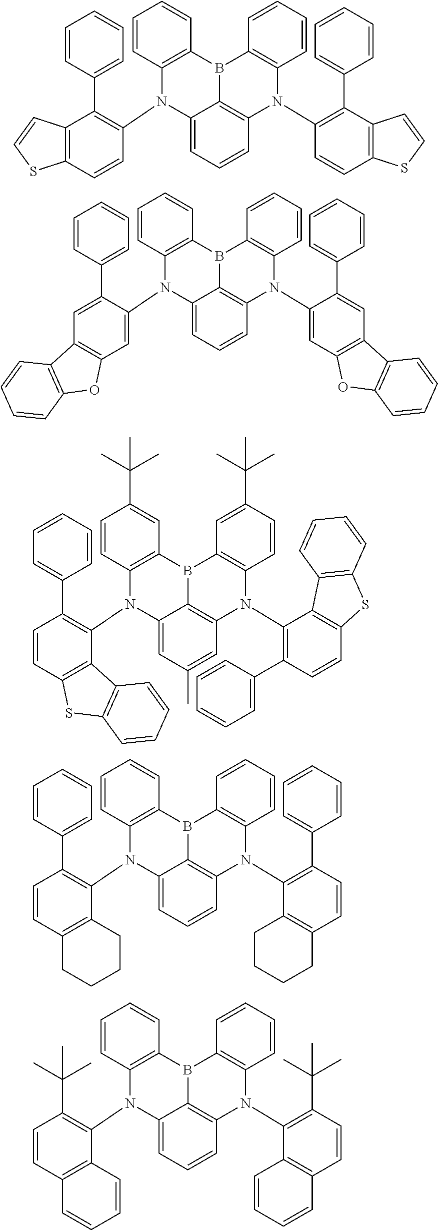

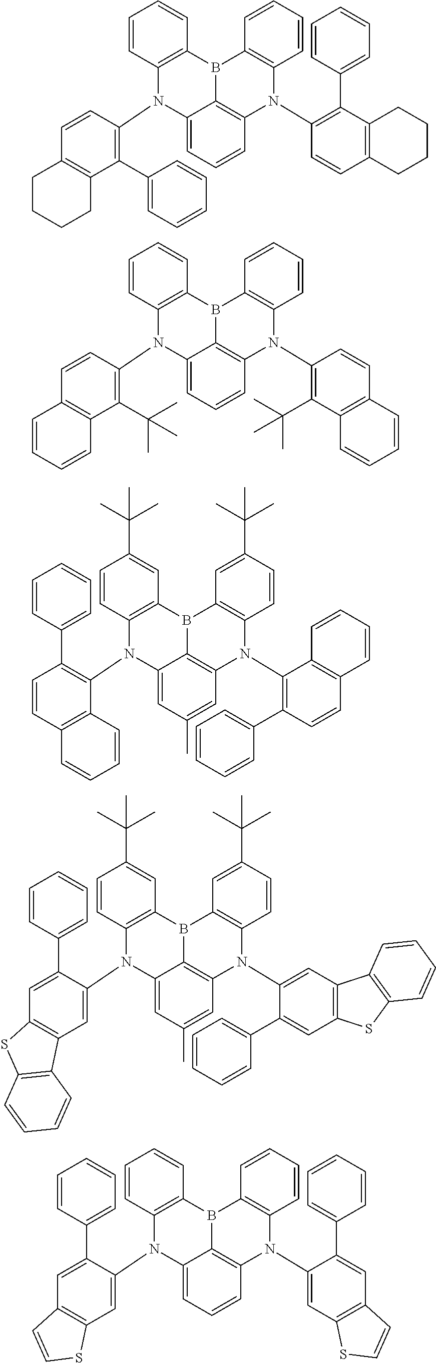

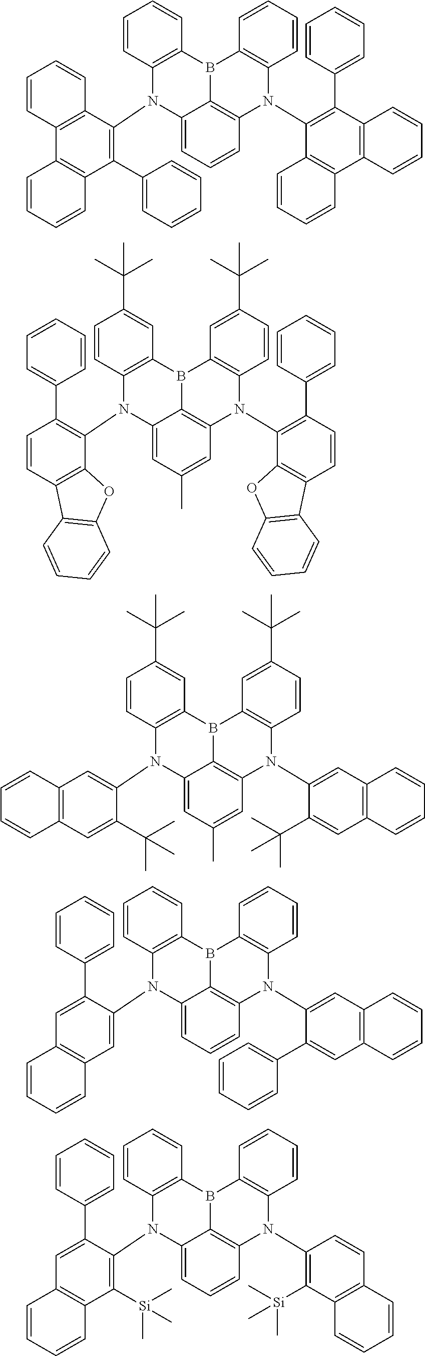

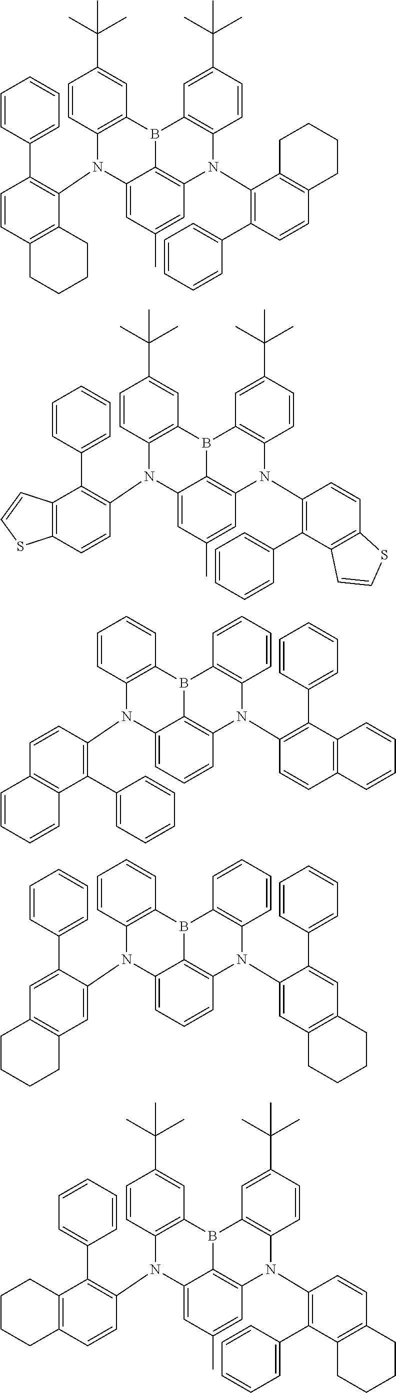

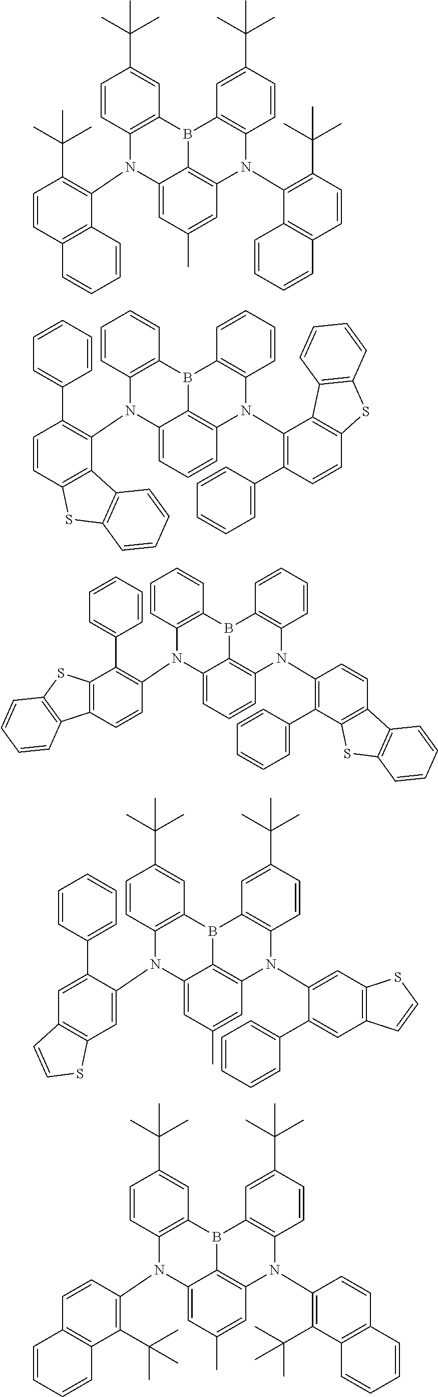

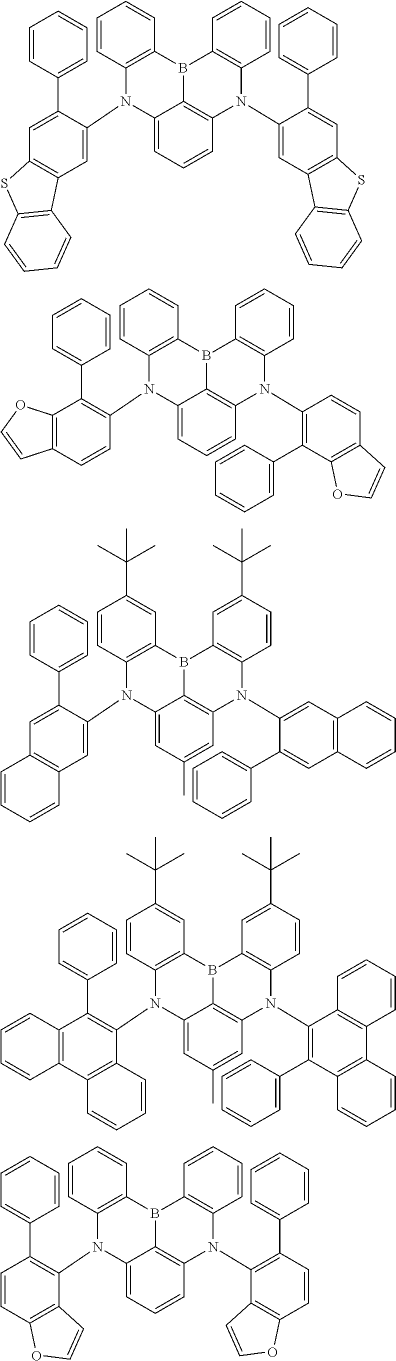

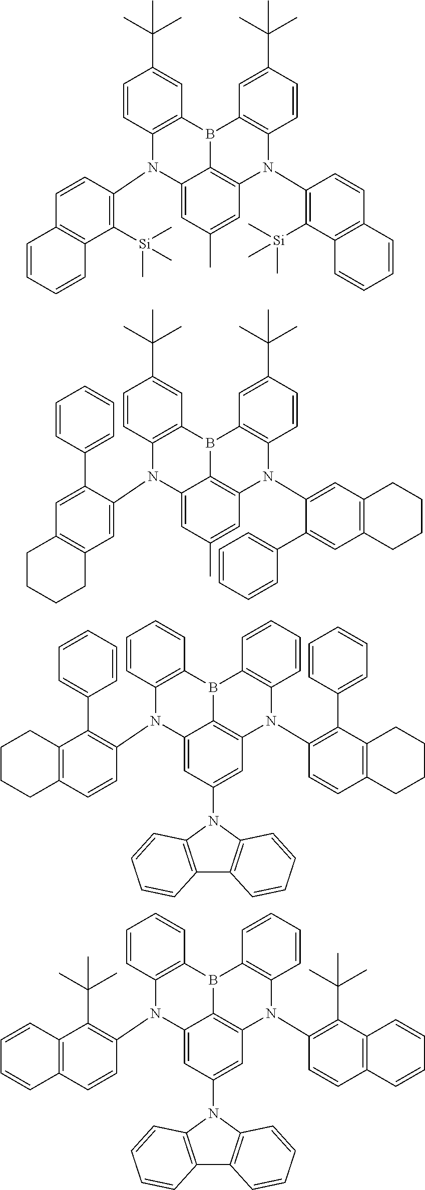

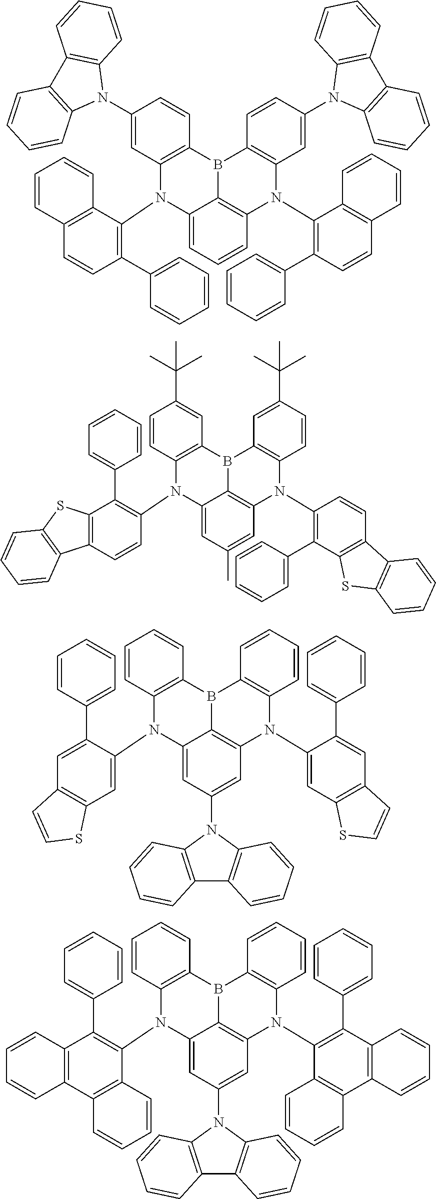

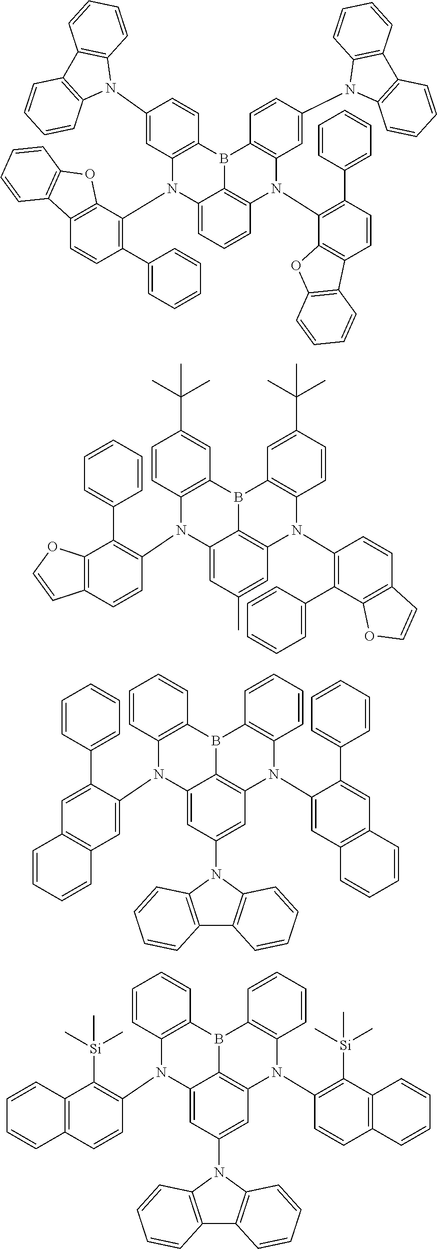

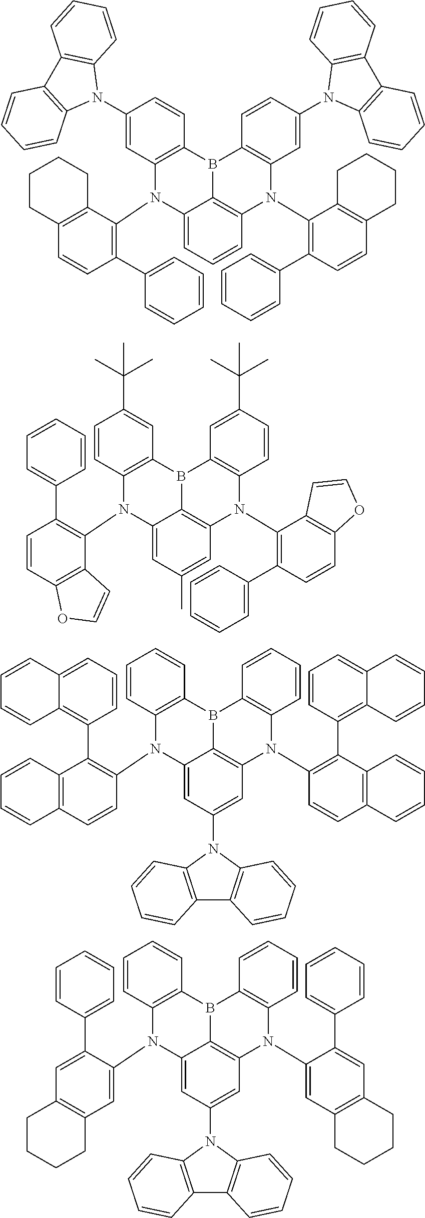

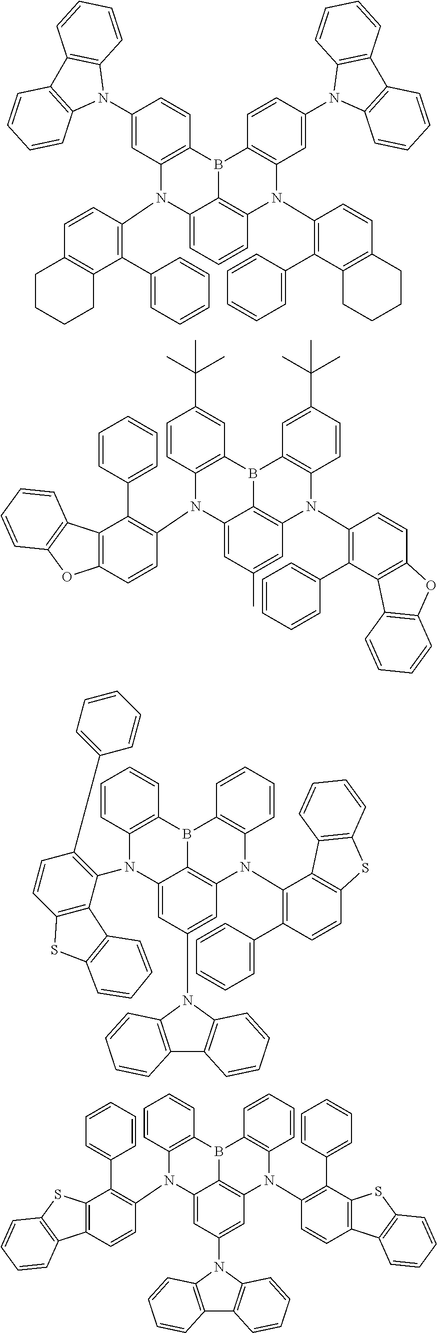

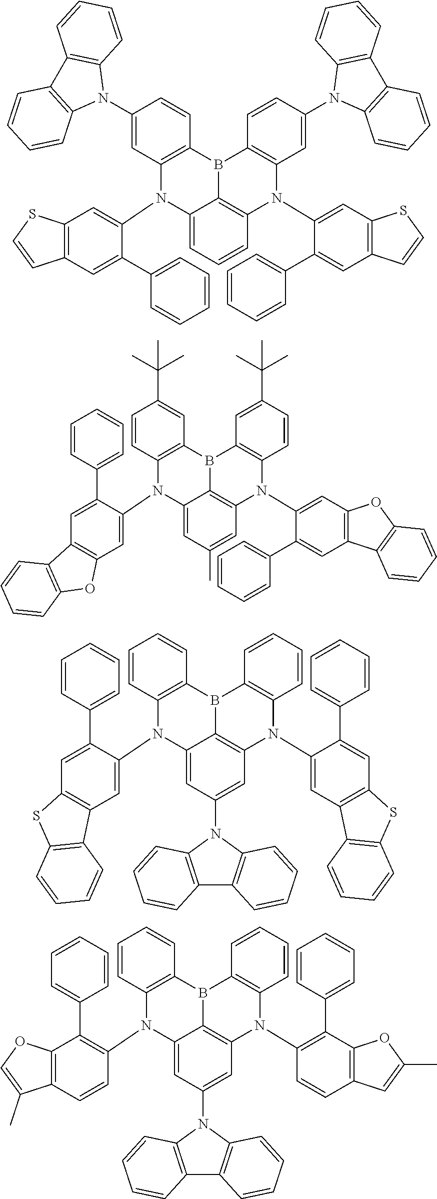

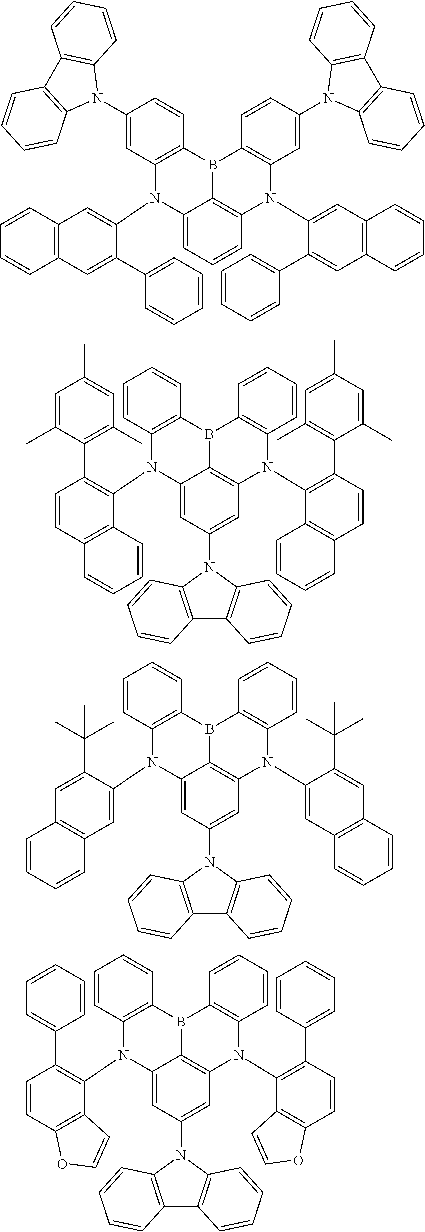

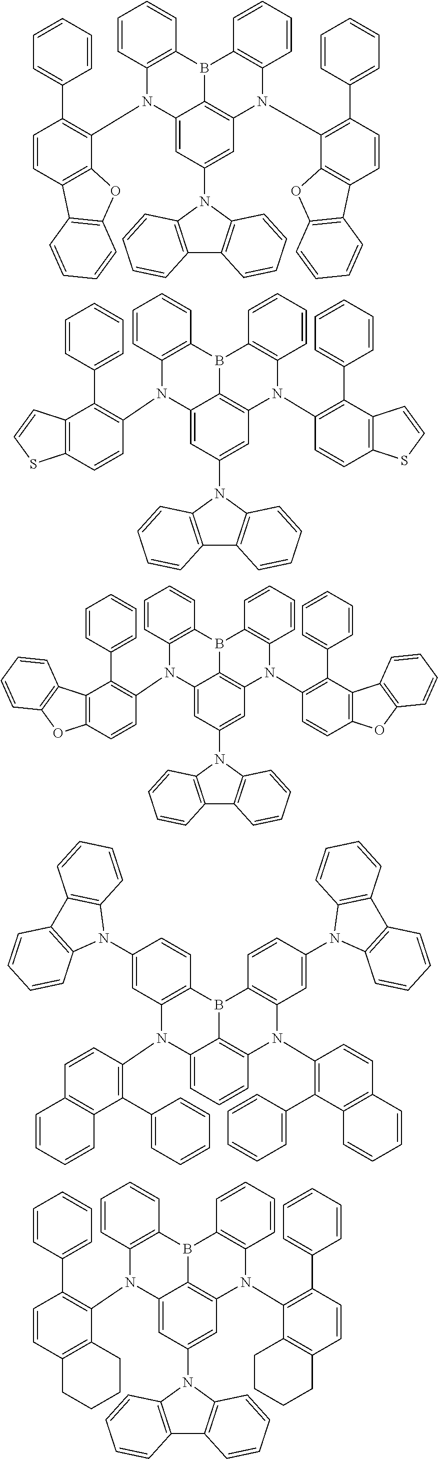

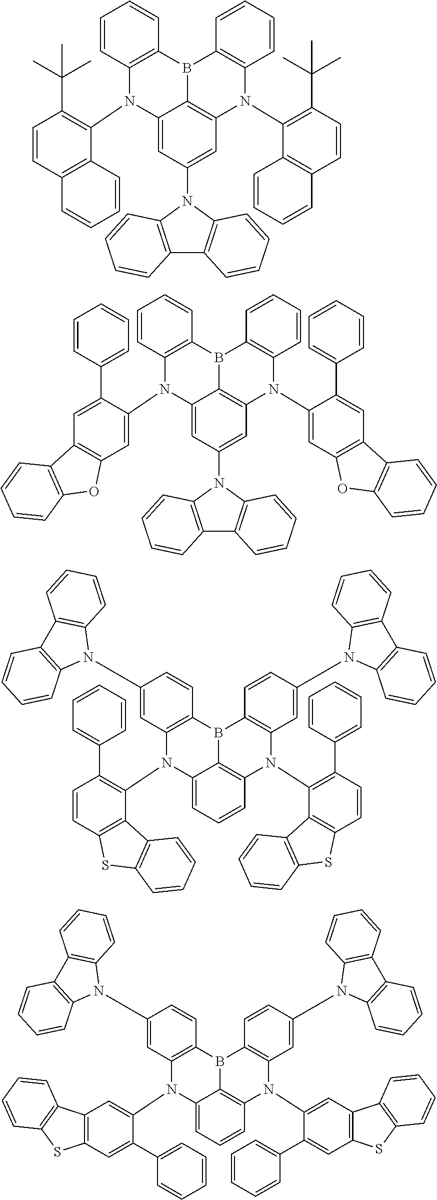

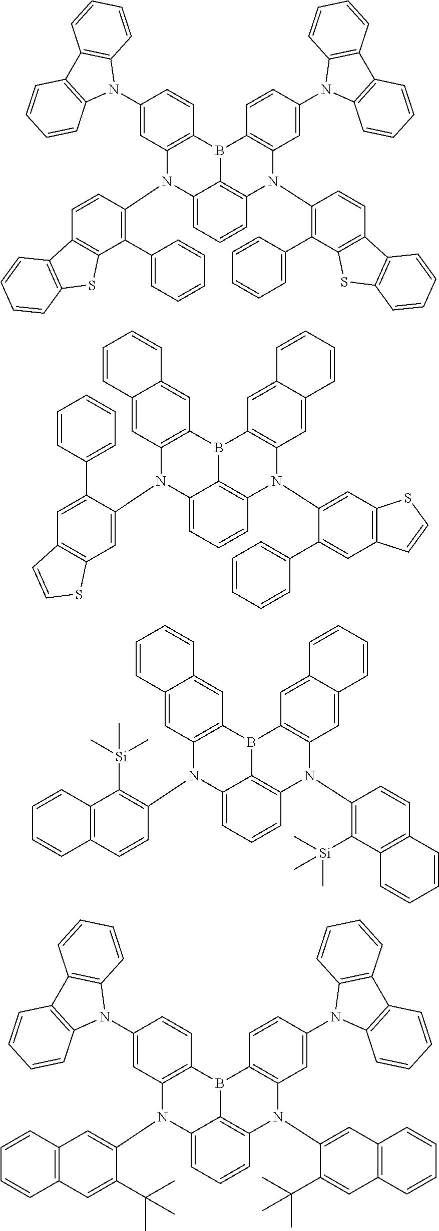

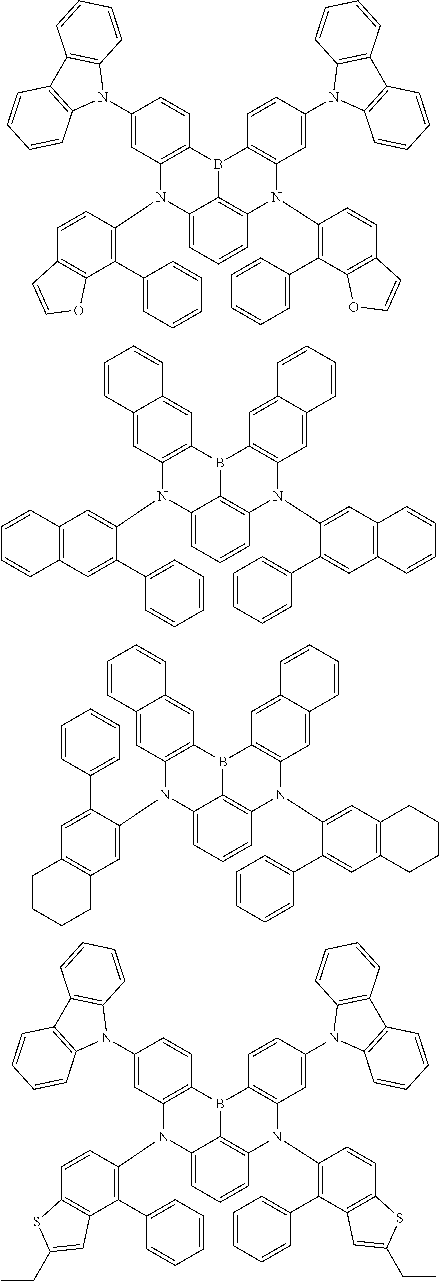

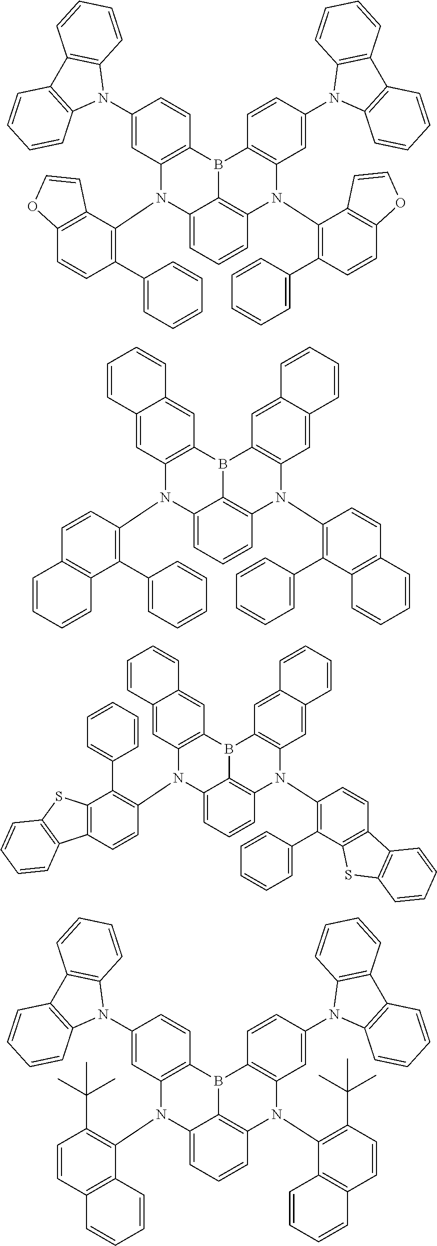

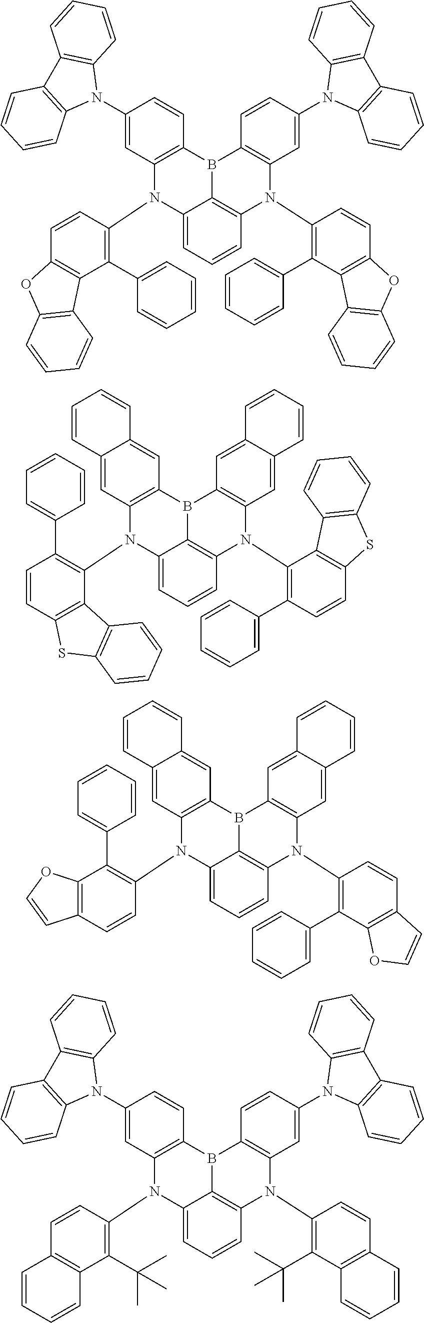

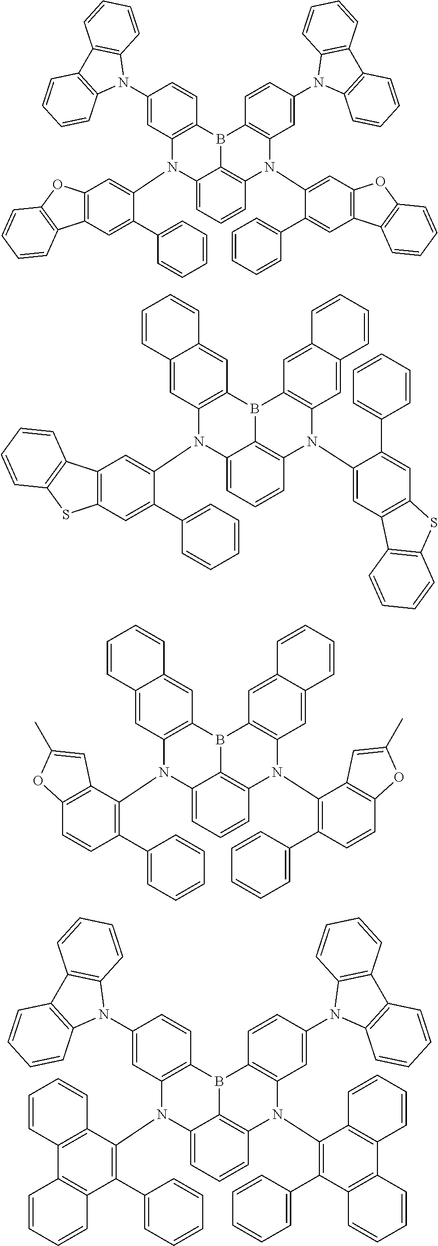

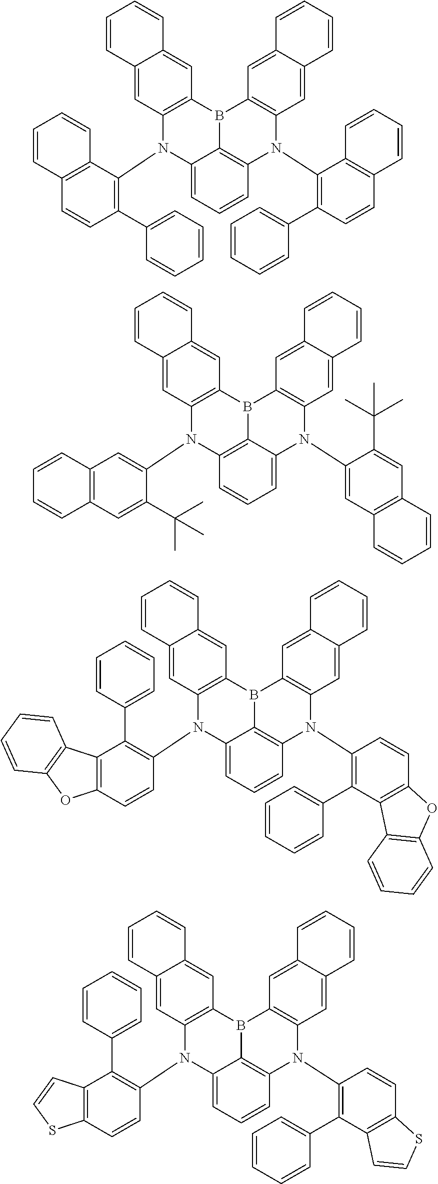

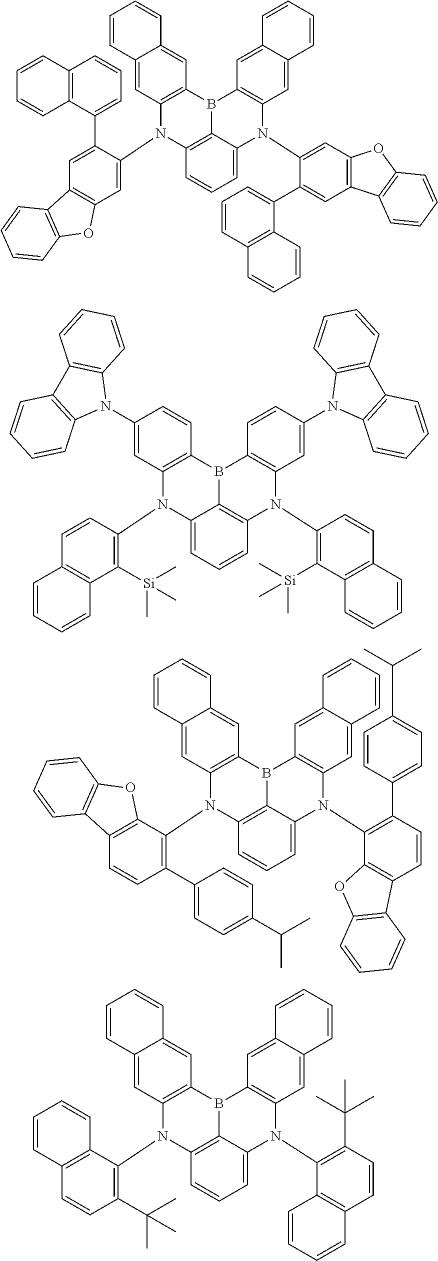

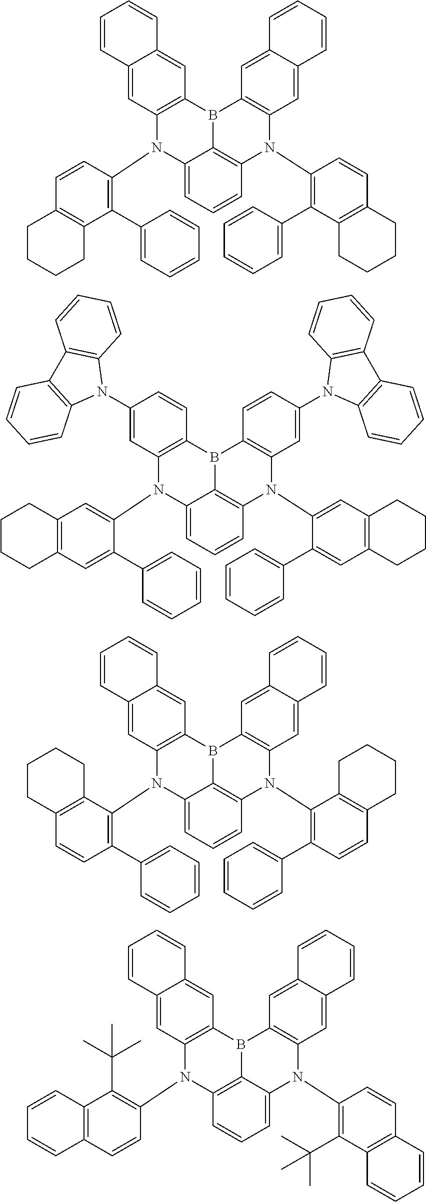

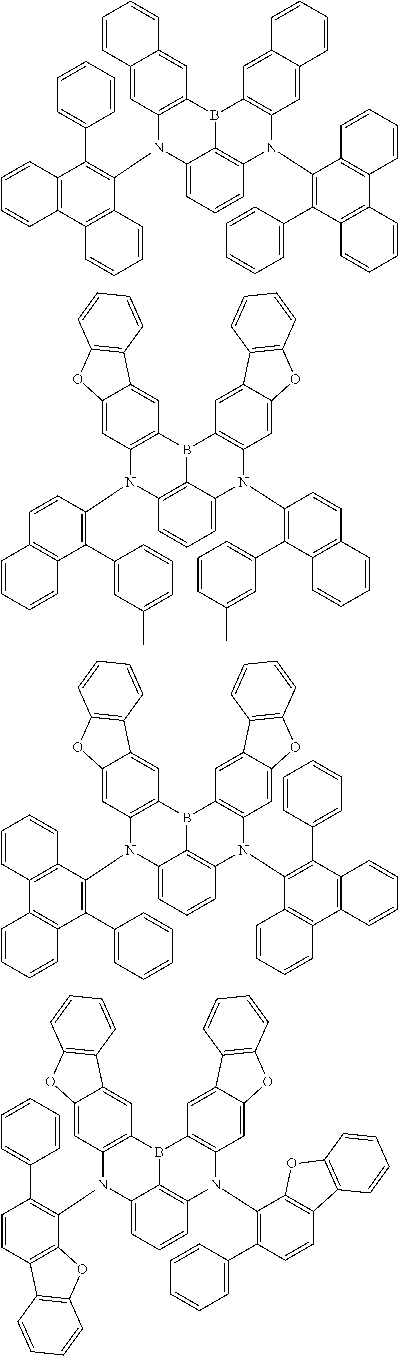

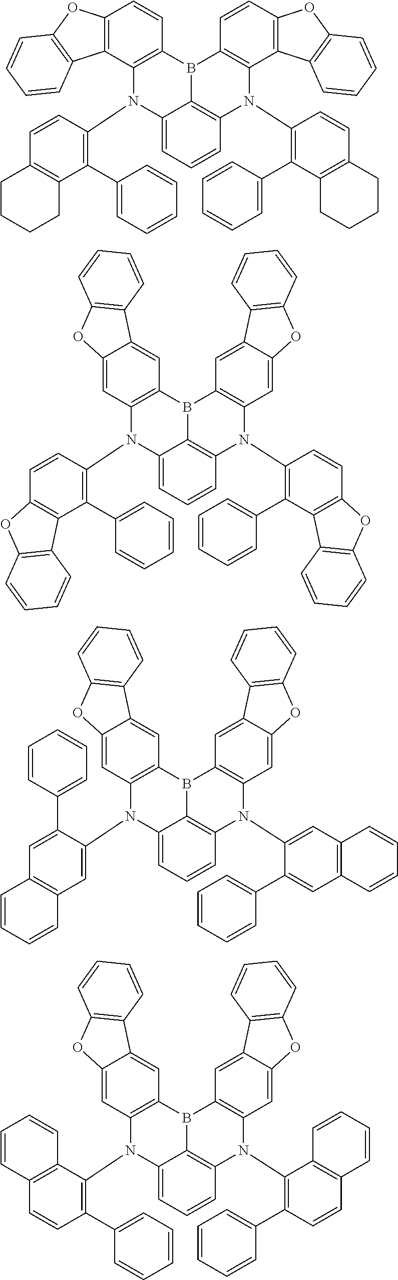

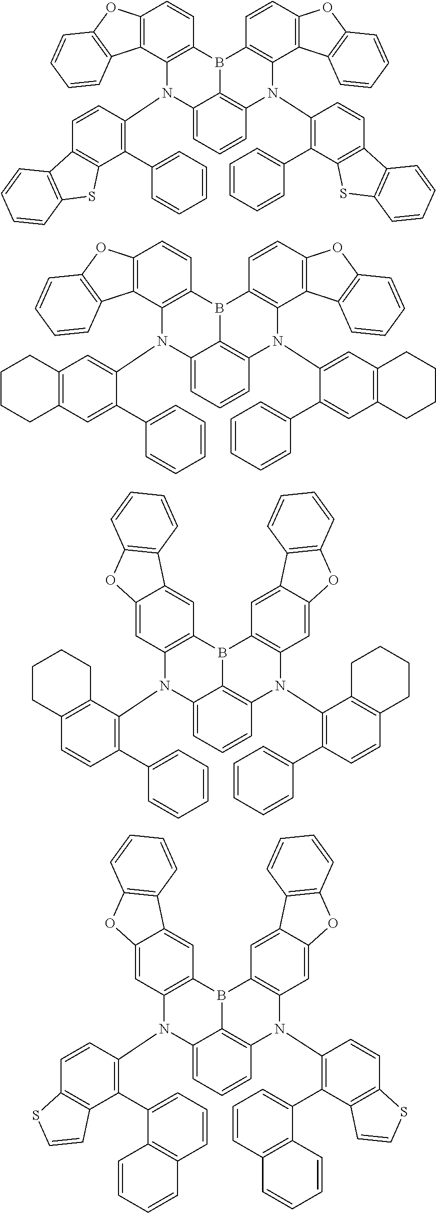

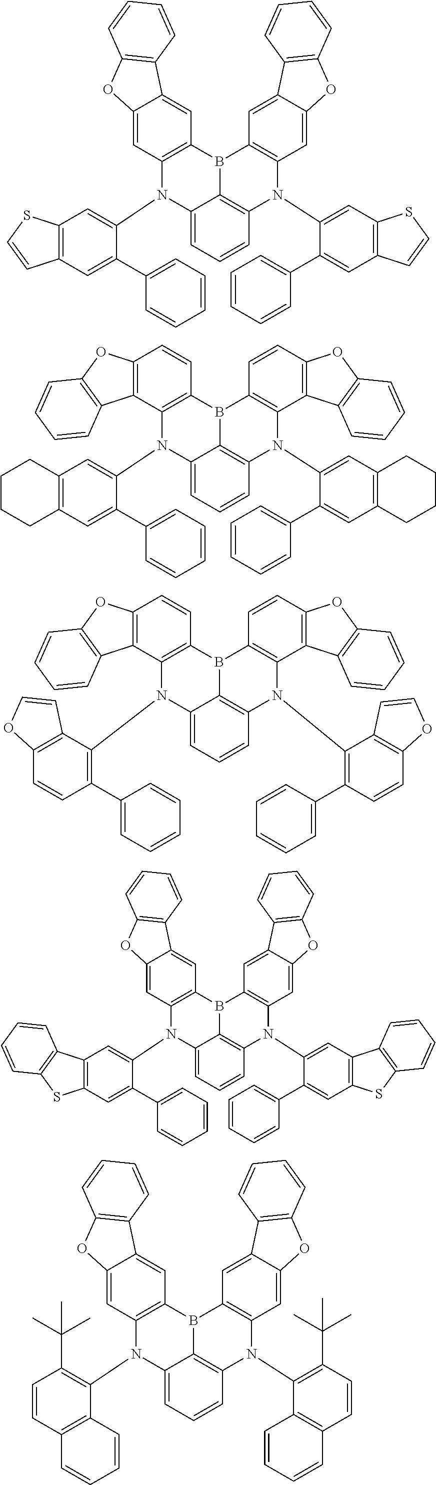

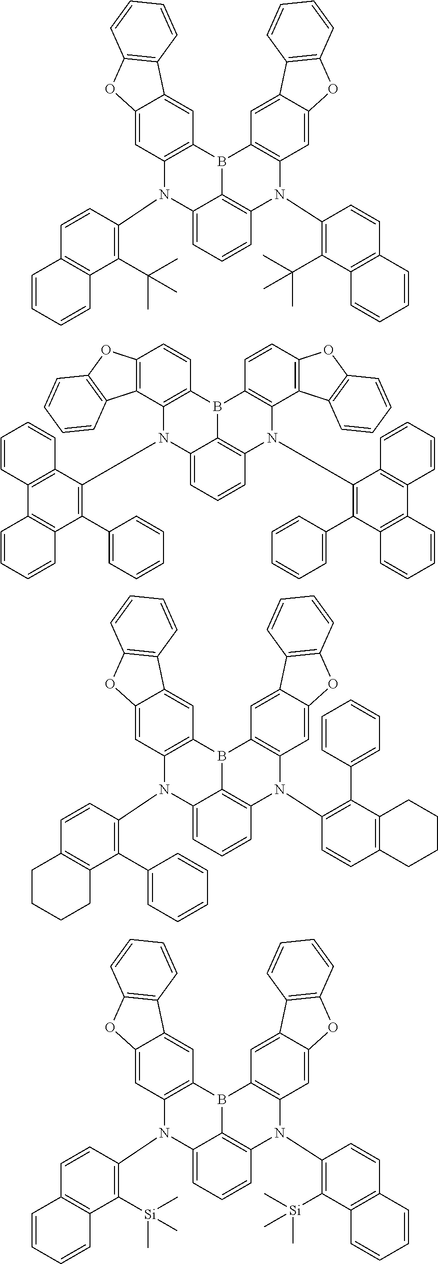

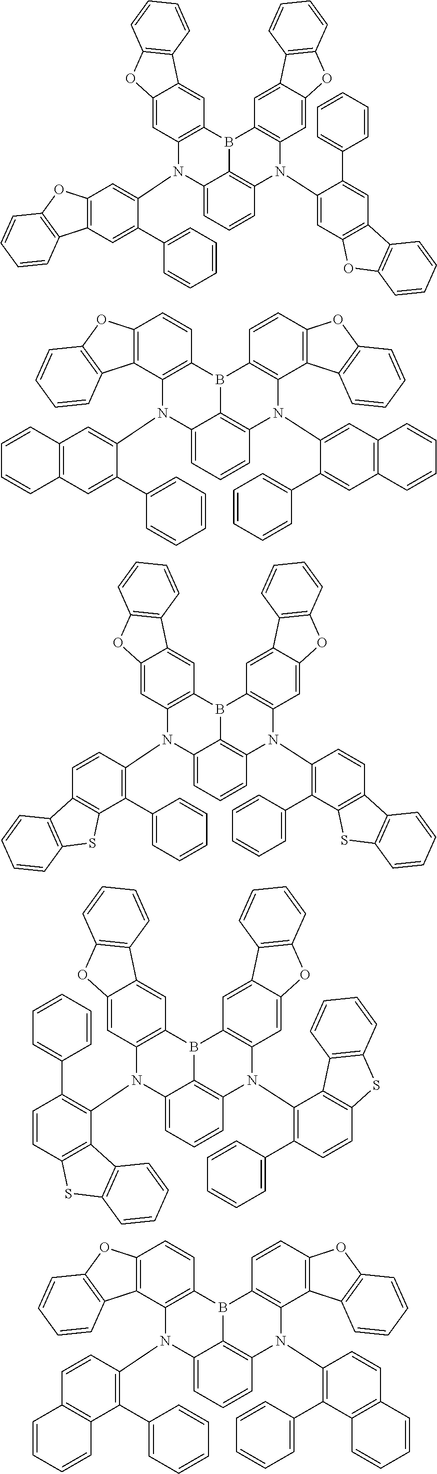

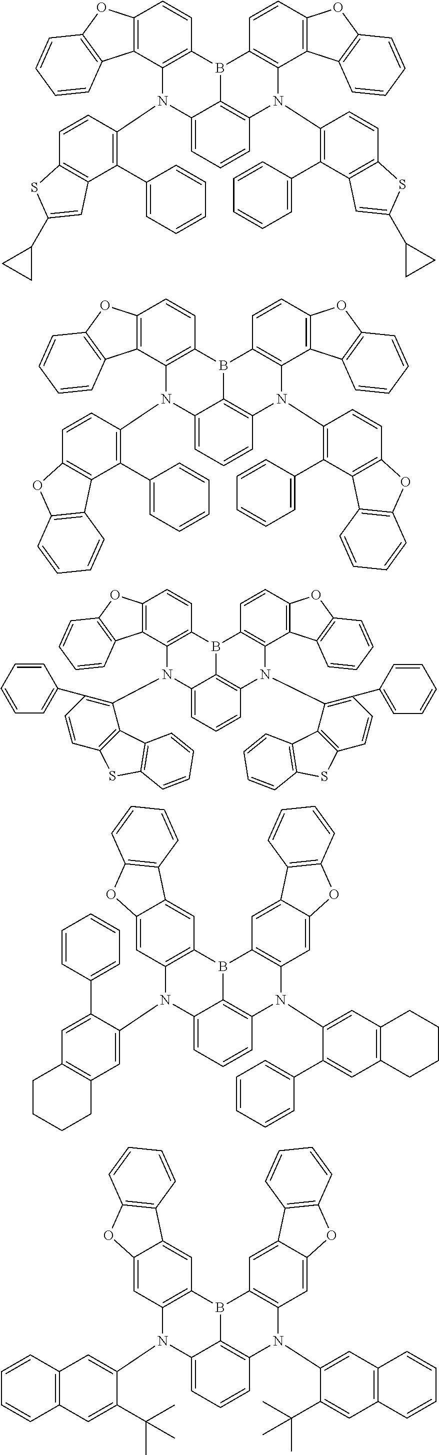

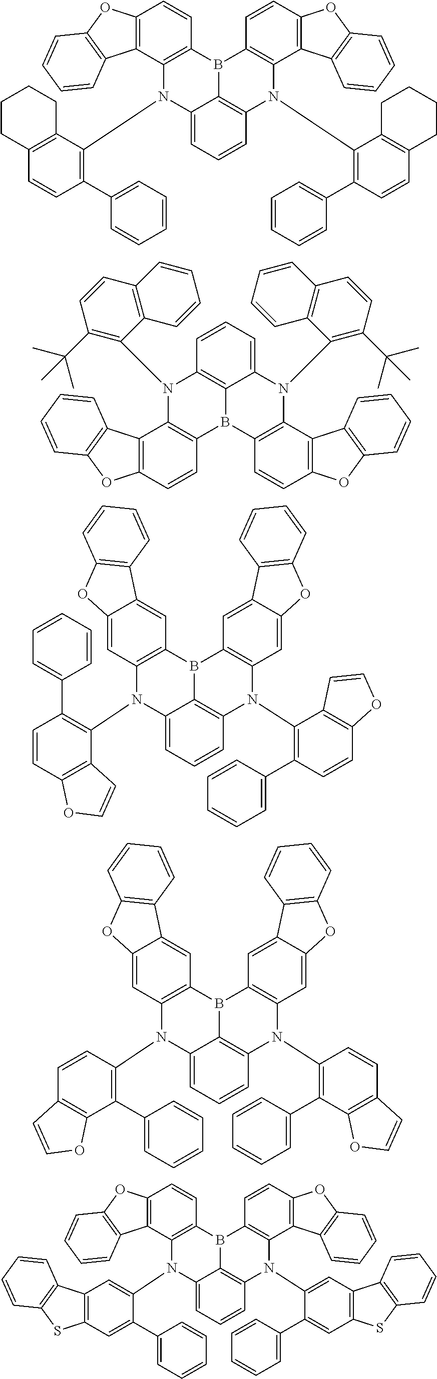

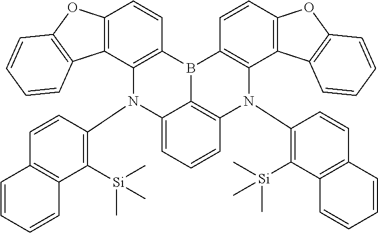 [SMILES: C[Si](C)(C)c1c(N2c3cccc4c3B(c3ccc5oc6ccccc6c5c32)c2ccc3oc5ccccc5c3c2N4c2ccc3ccccc3c2[Si](C)(C)C)ccc2ccccc12]

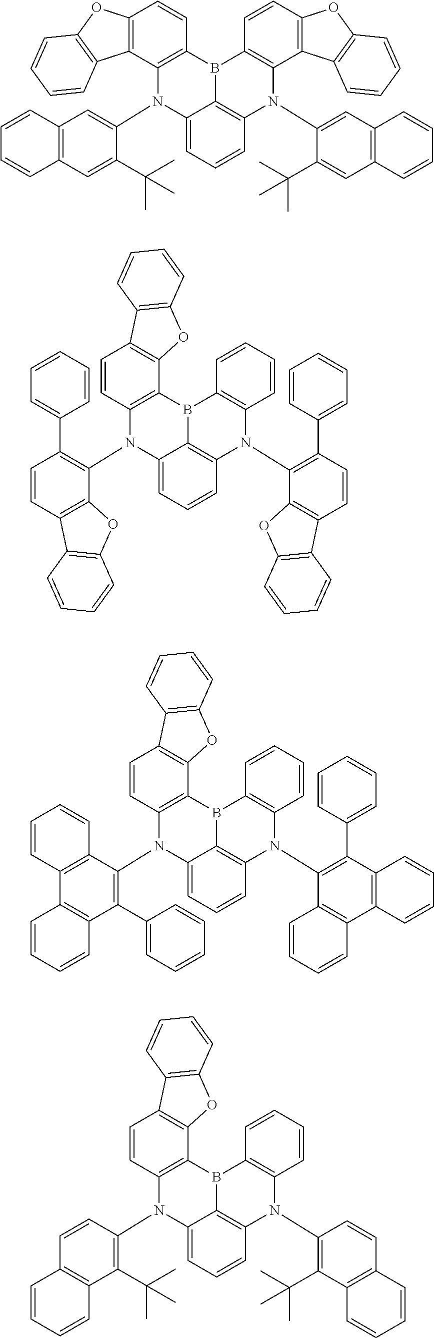

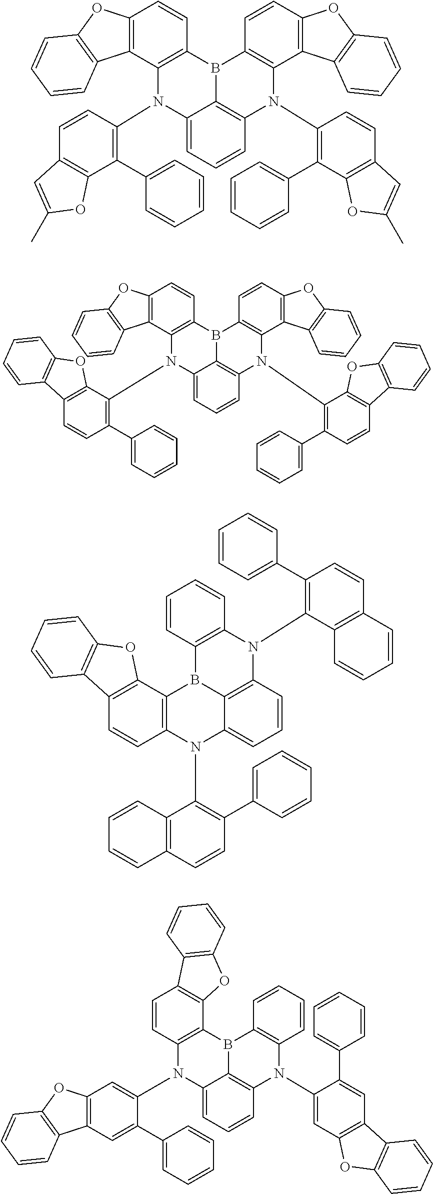

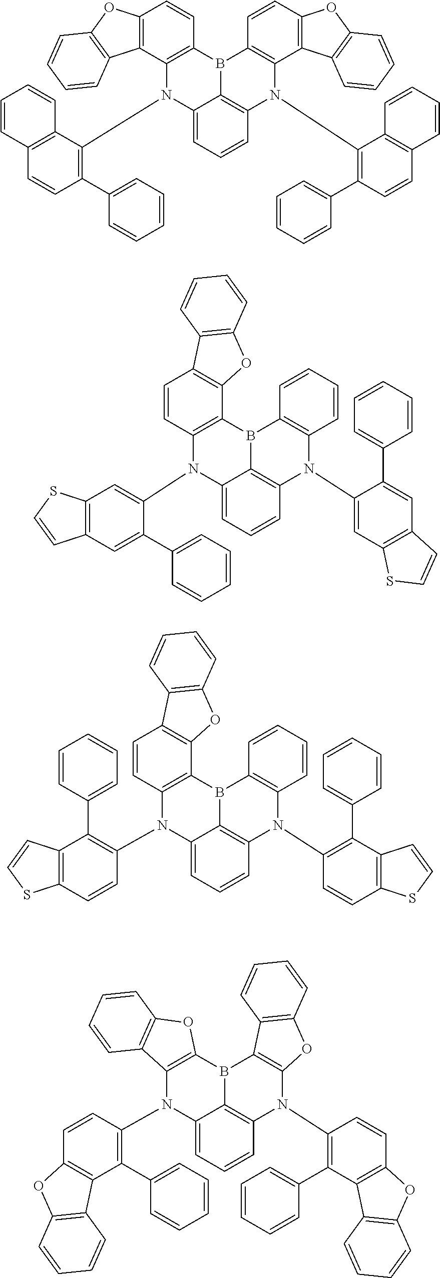

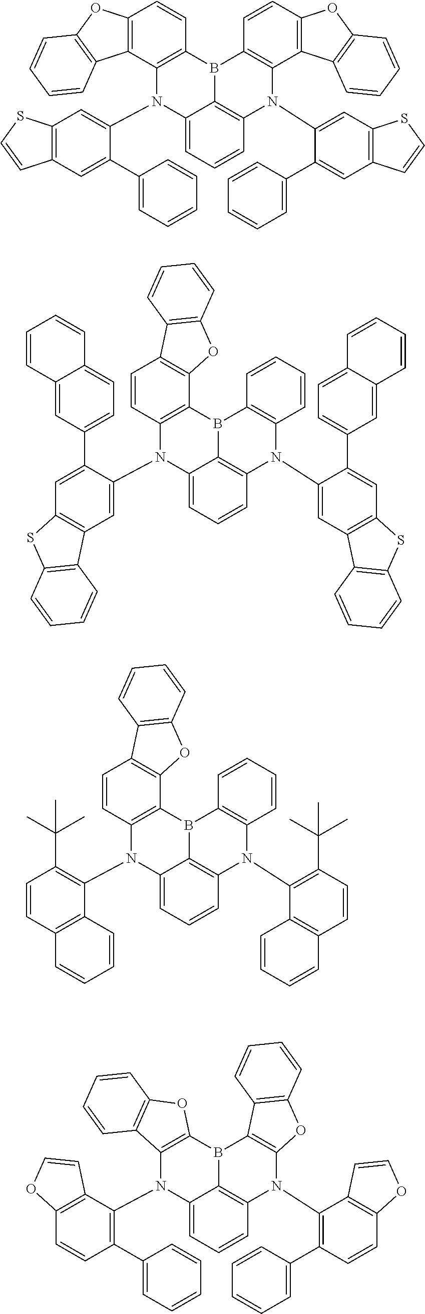

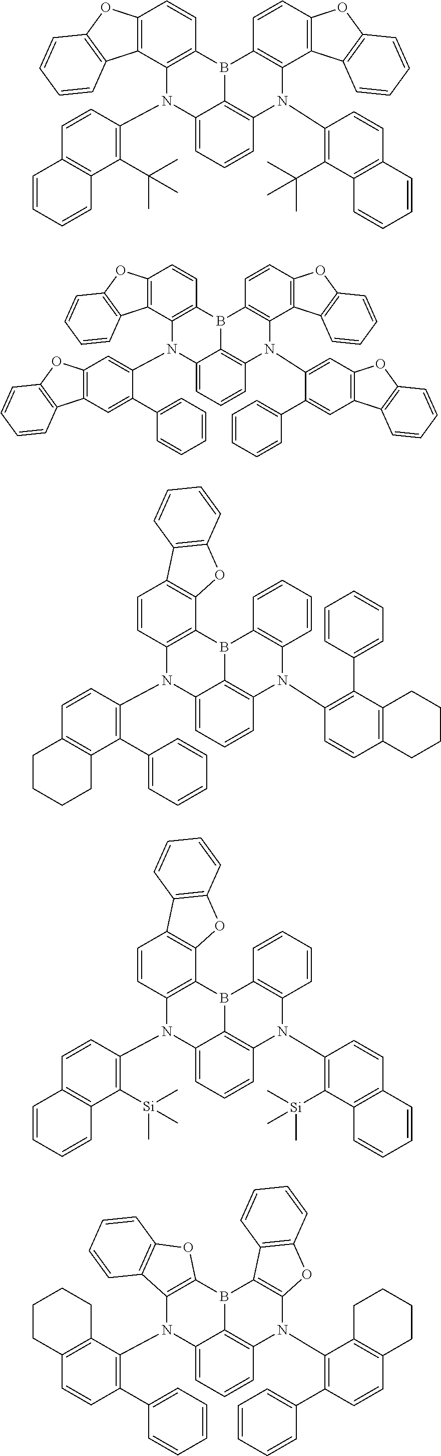

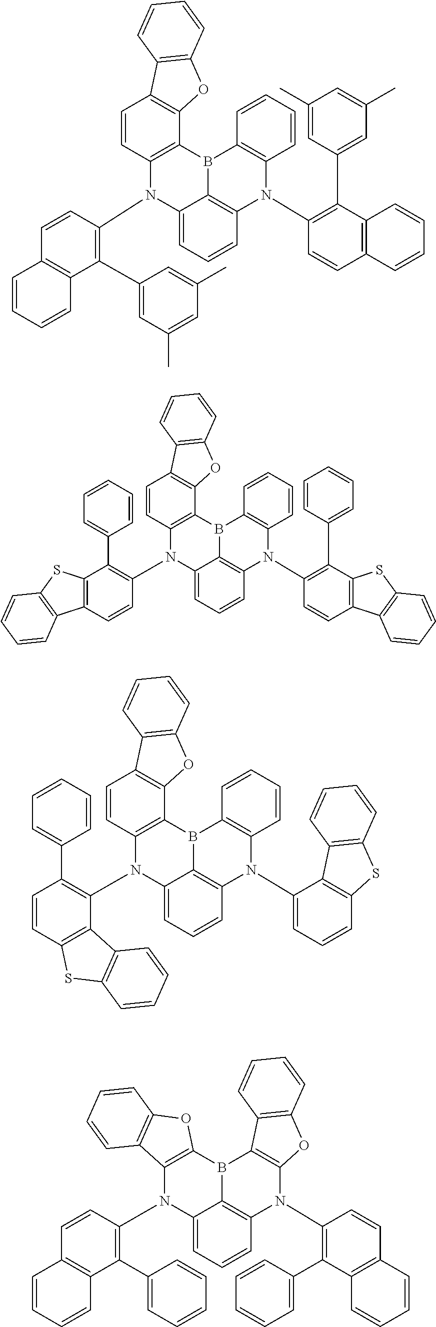

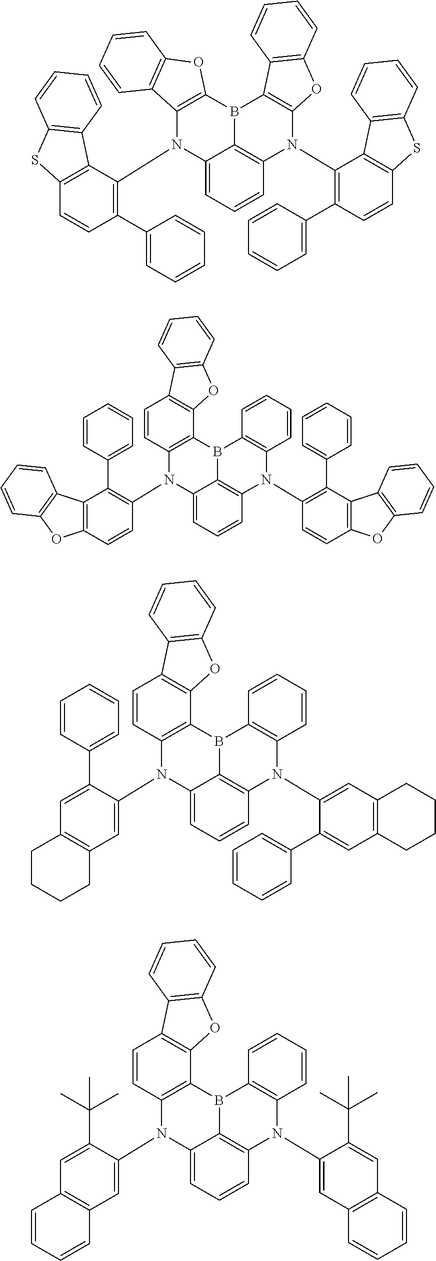

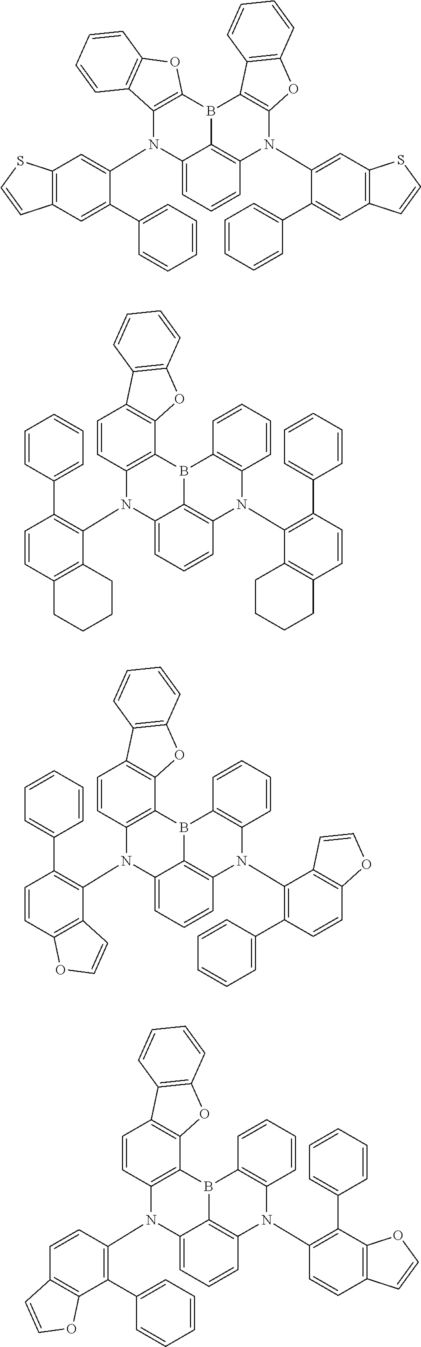

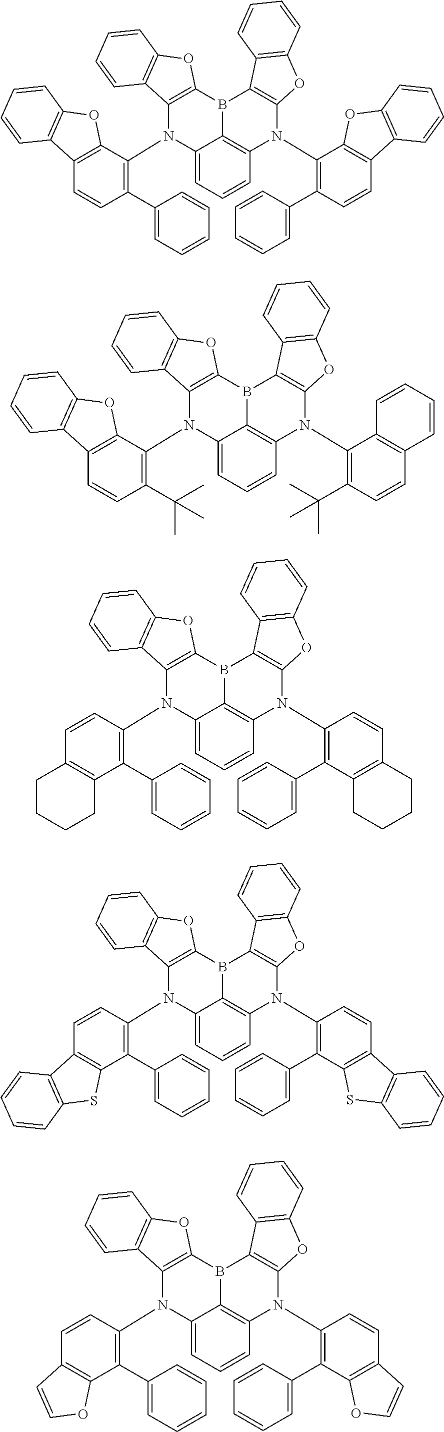

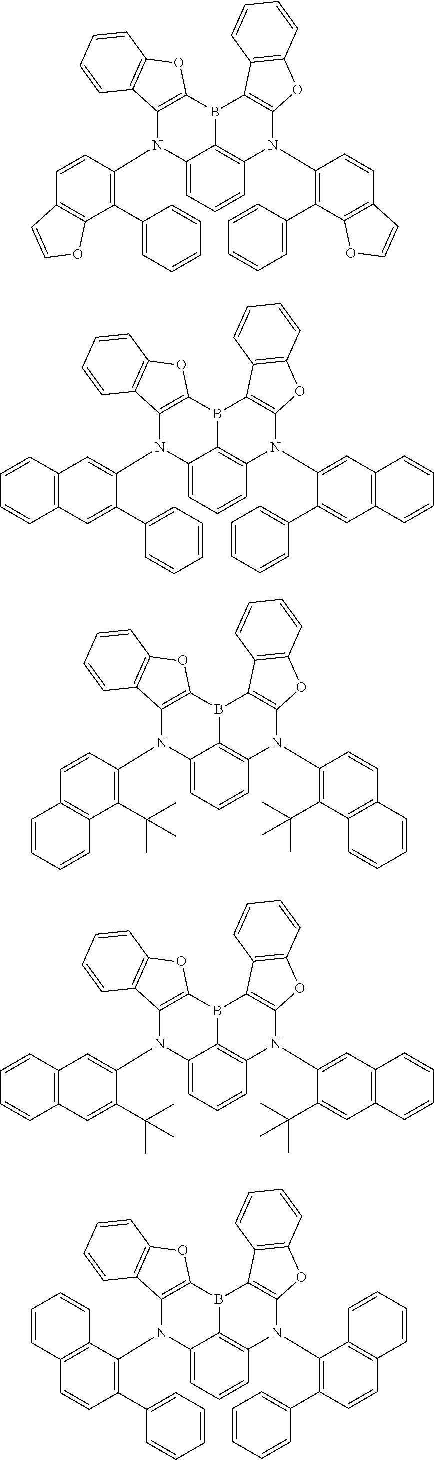

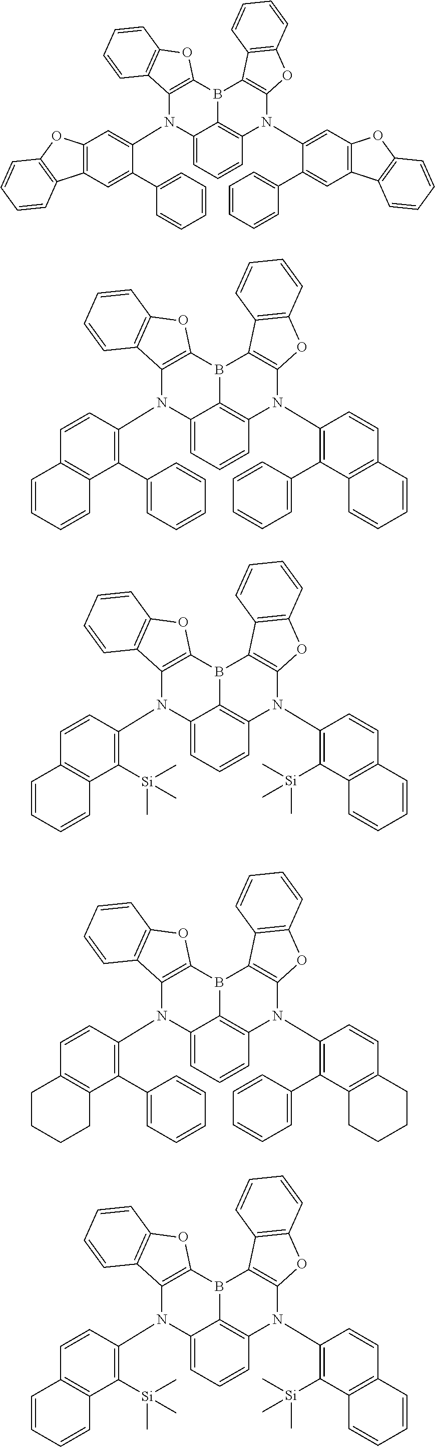

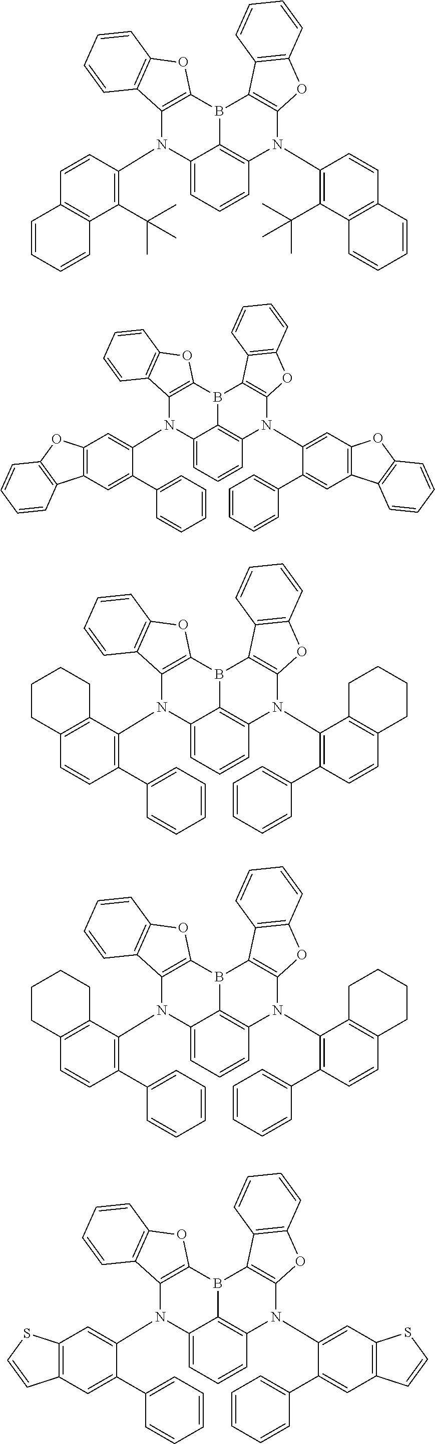

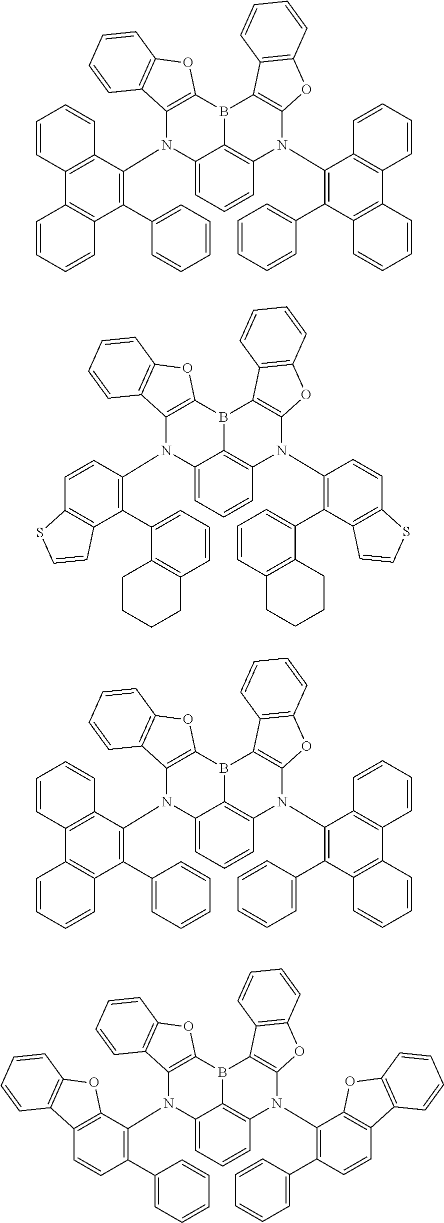

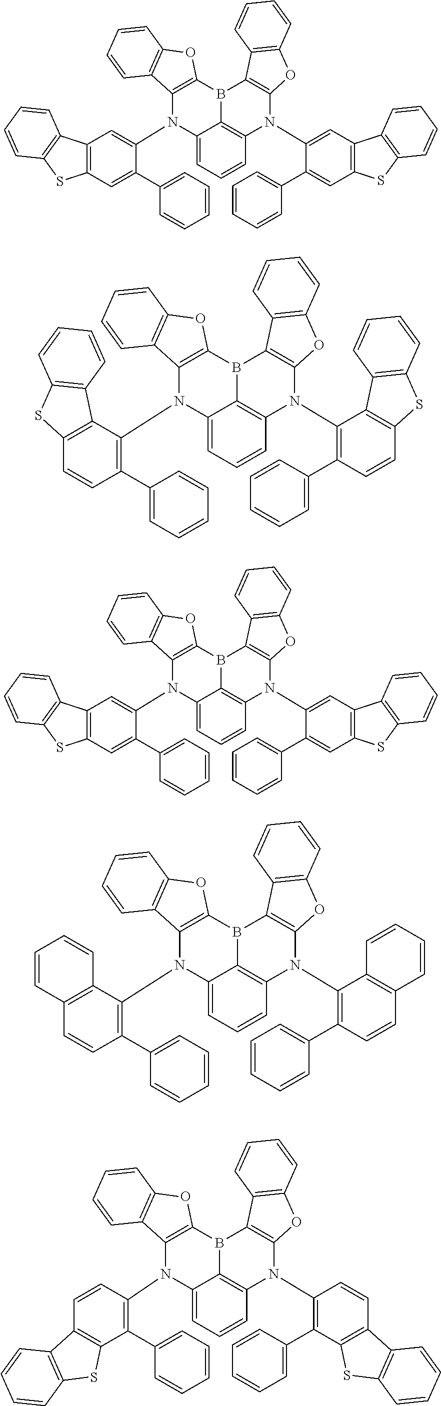

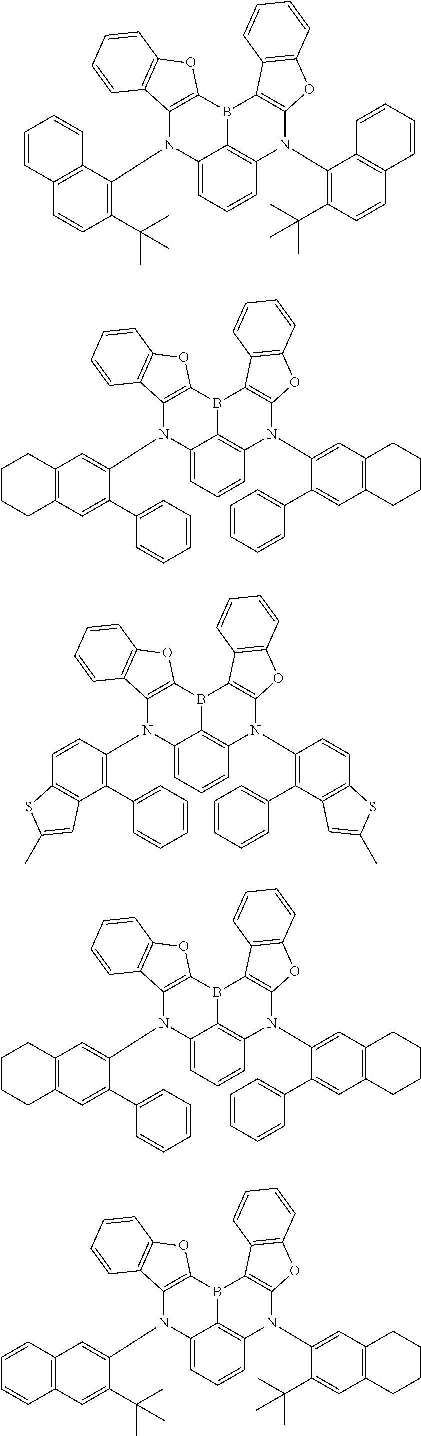

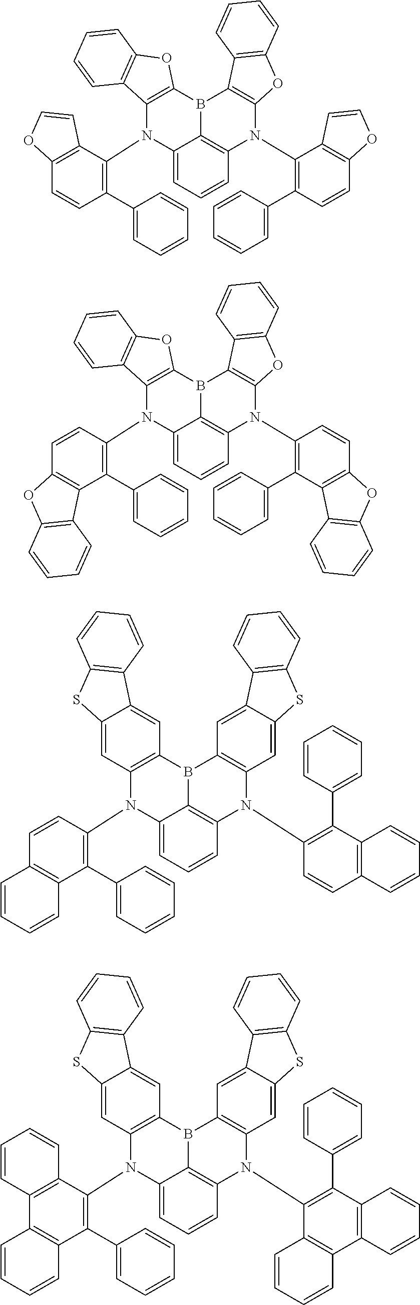

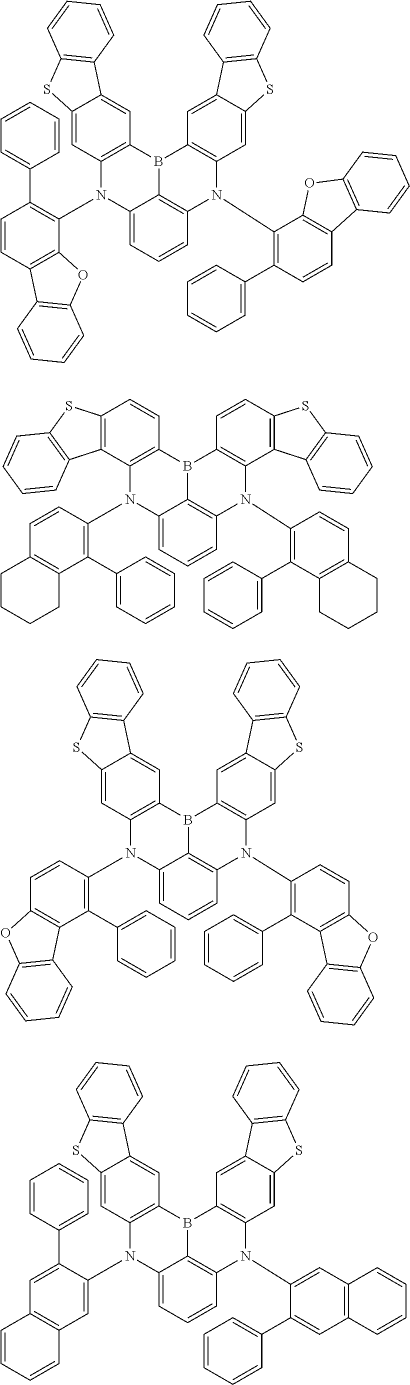

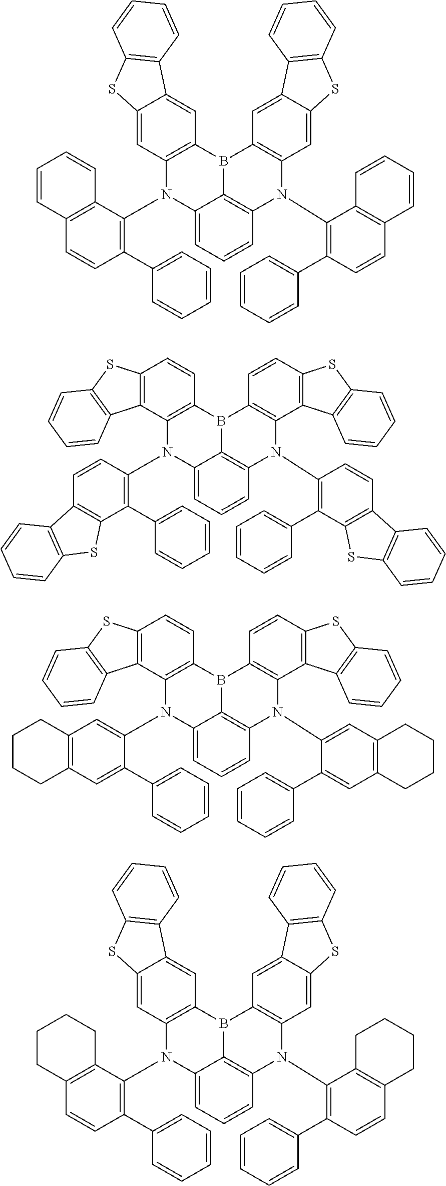

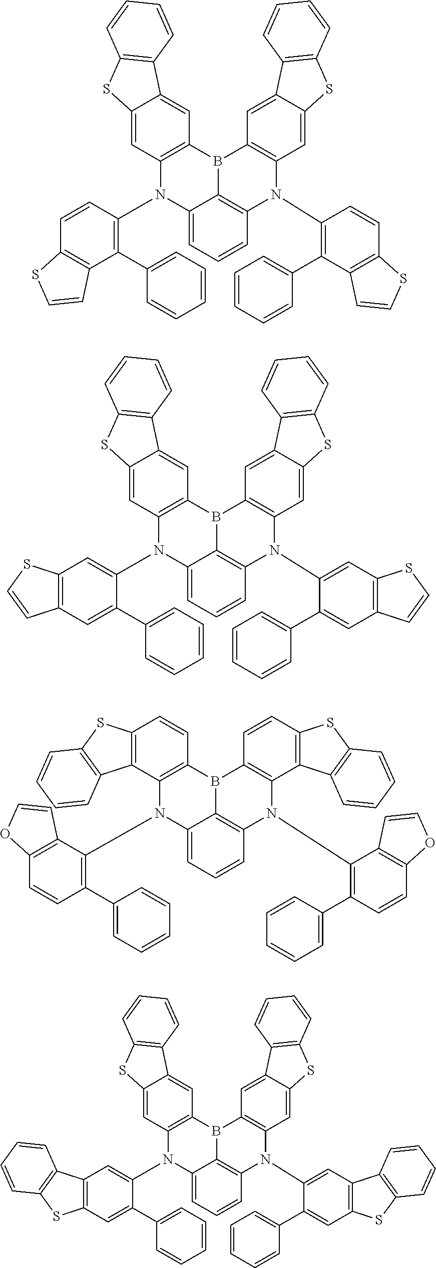

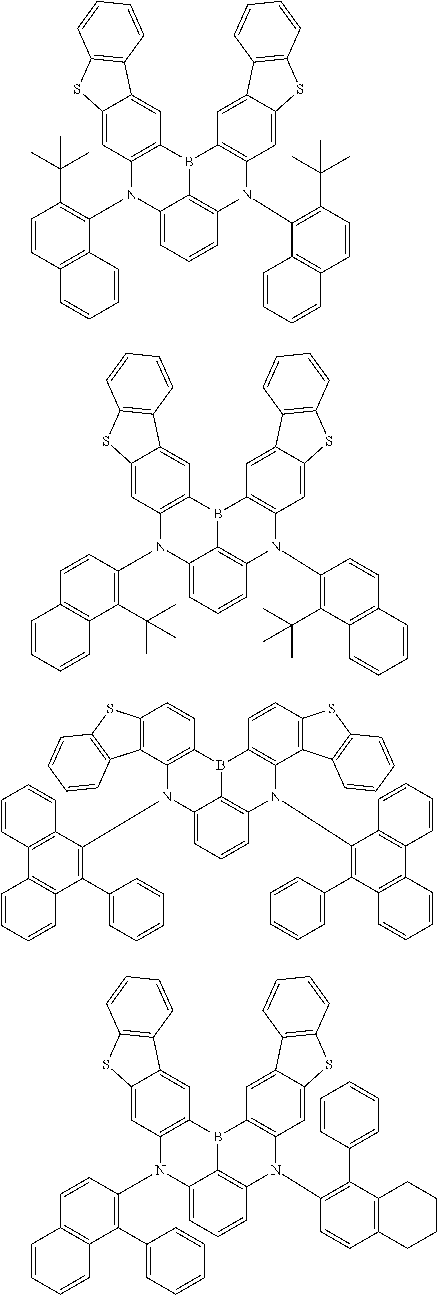

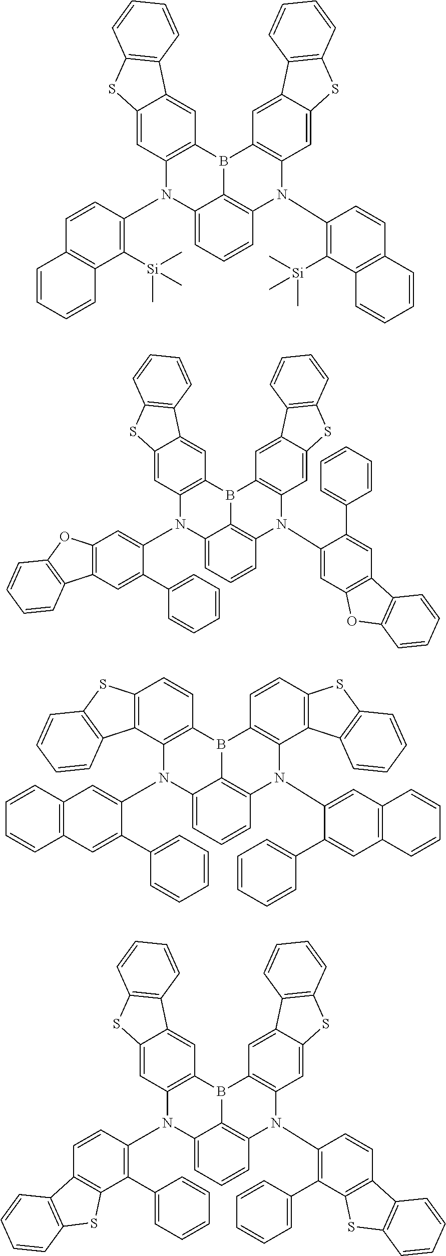

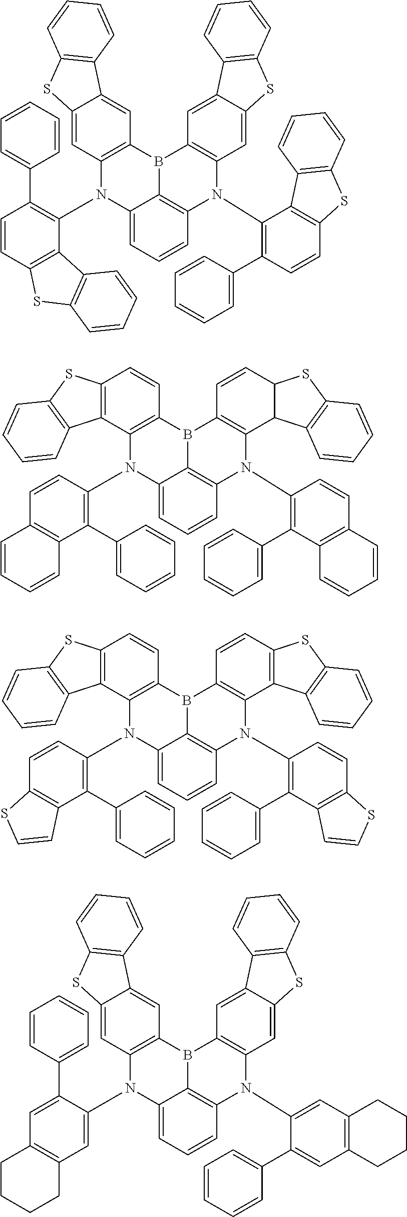

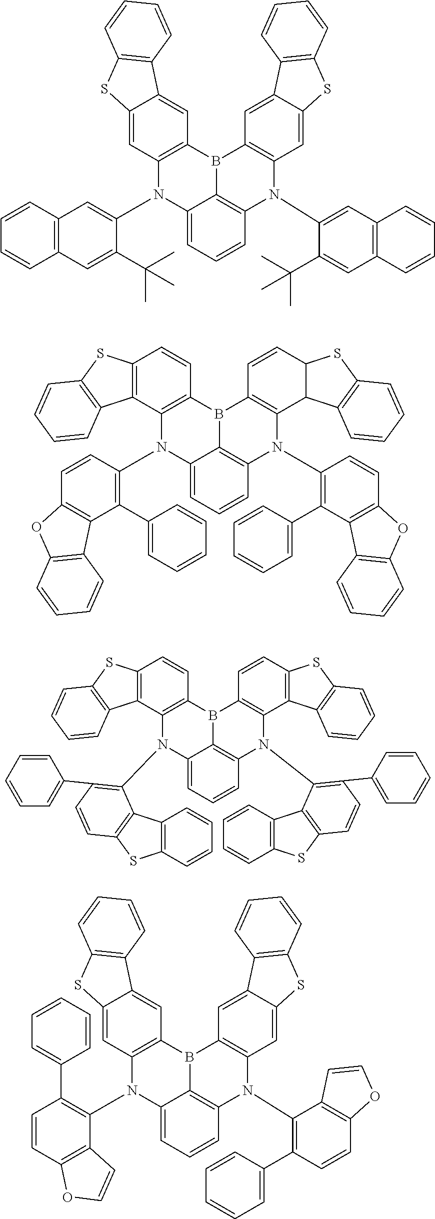

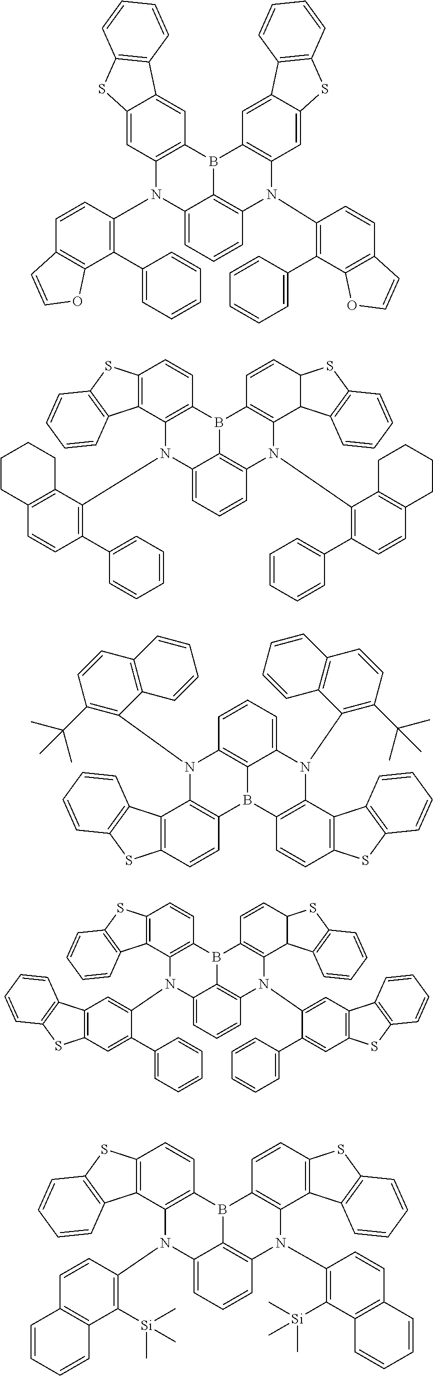

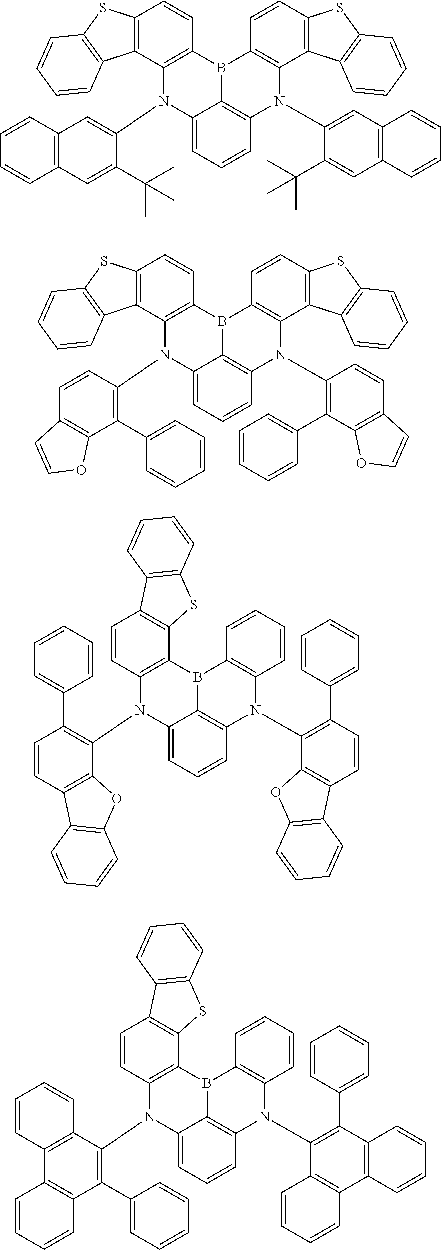

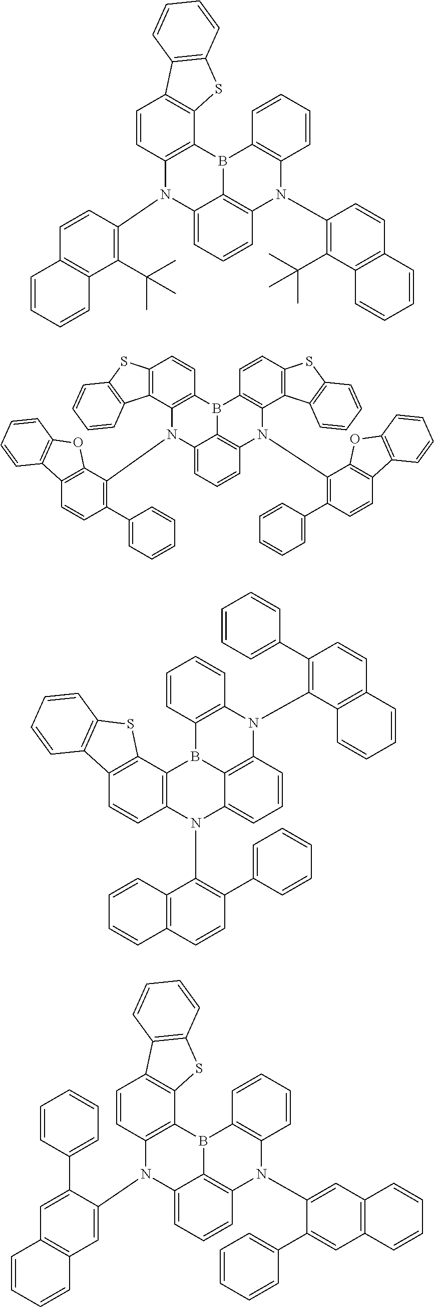

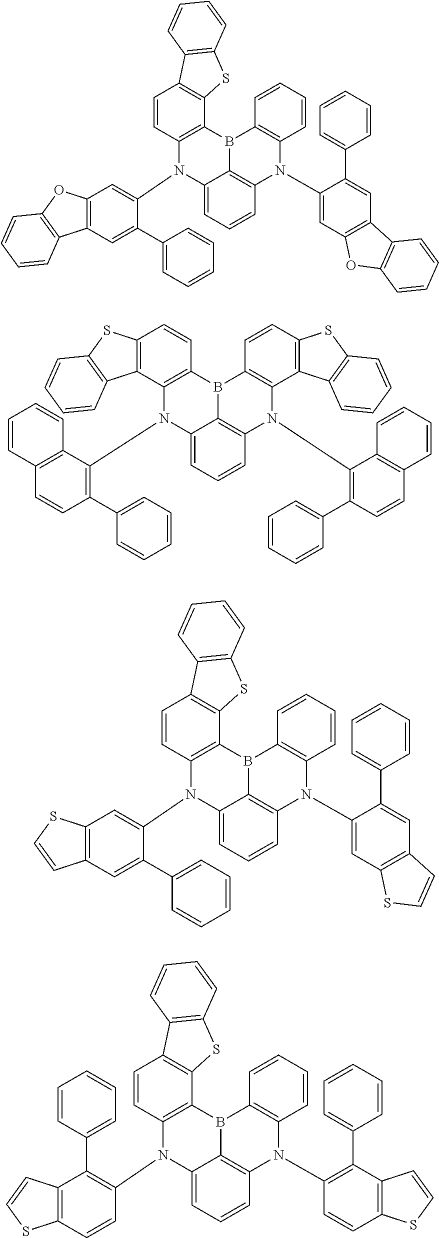

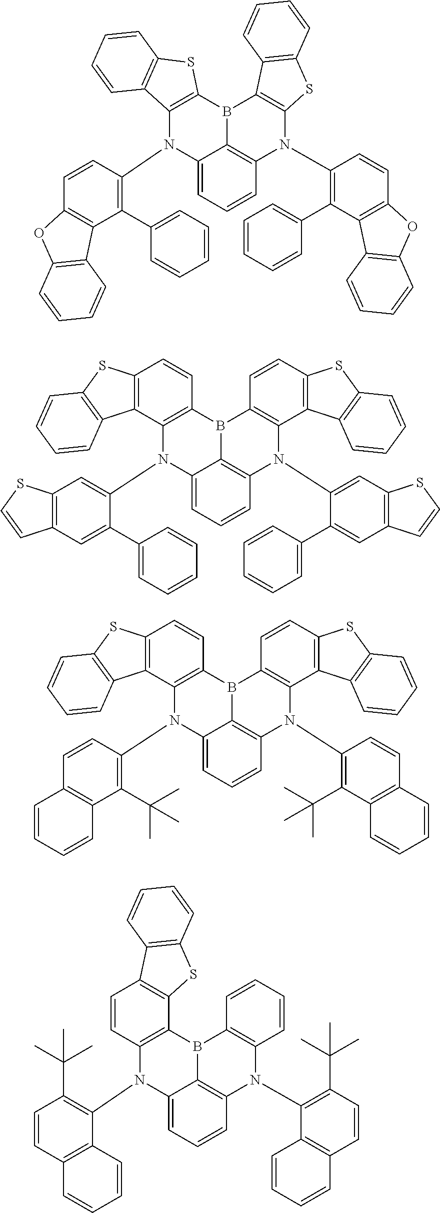

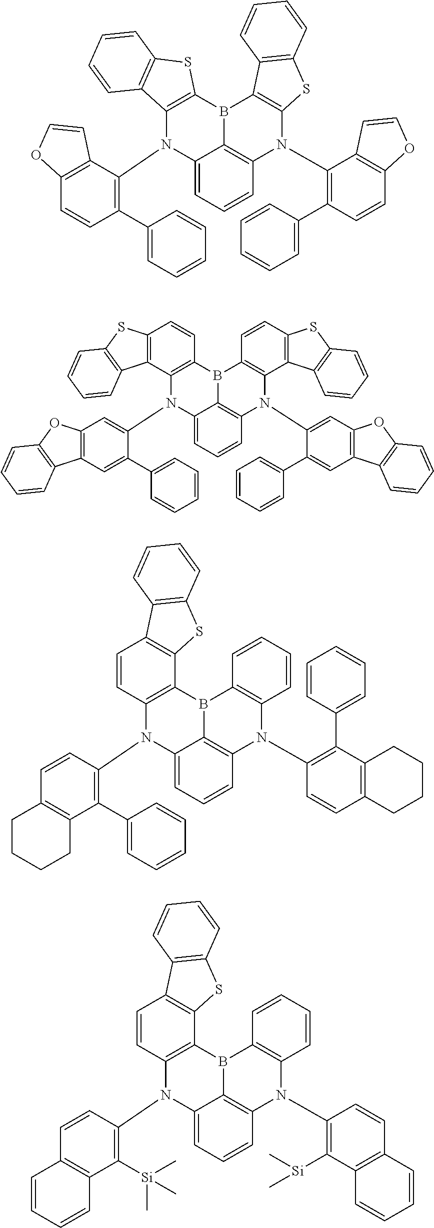

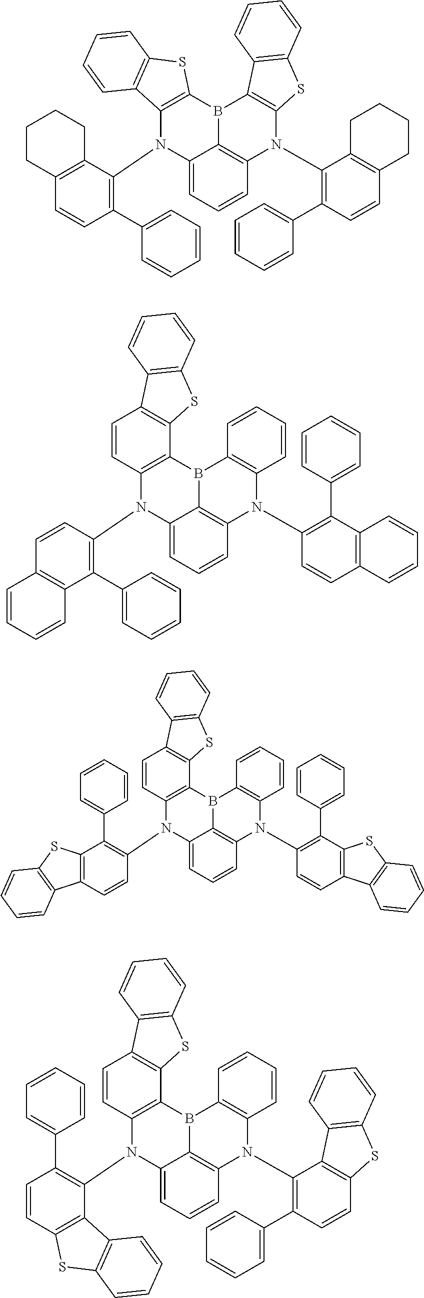

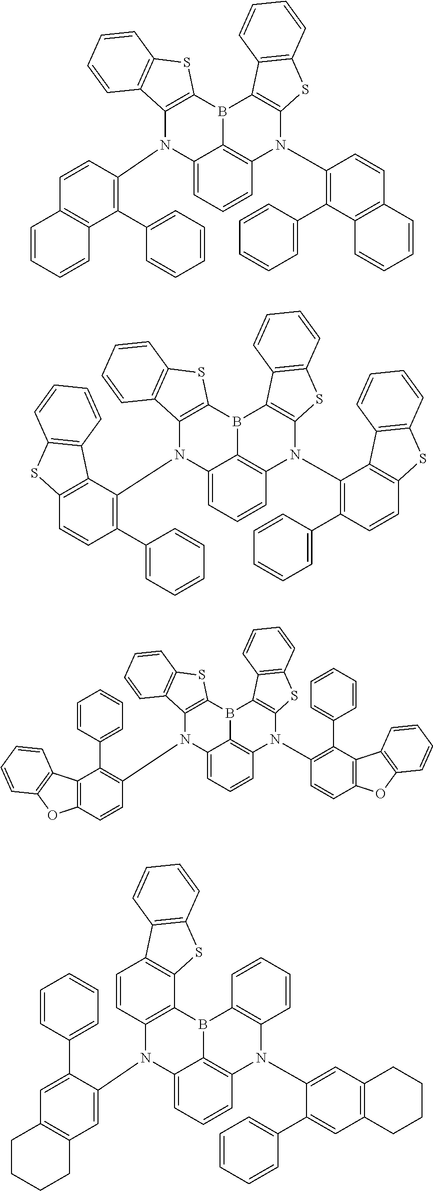

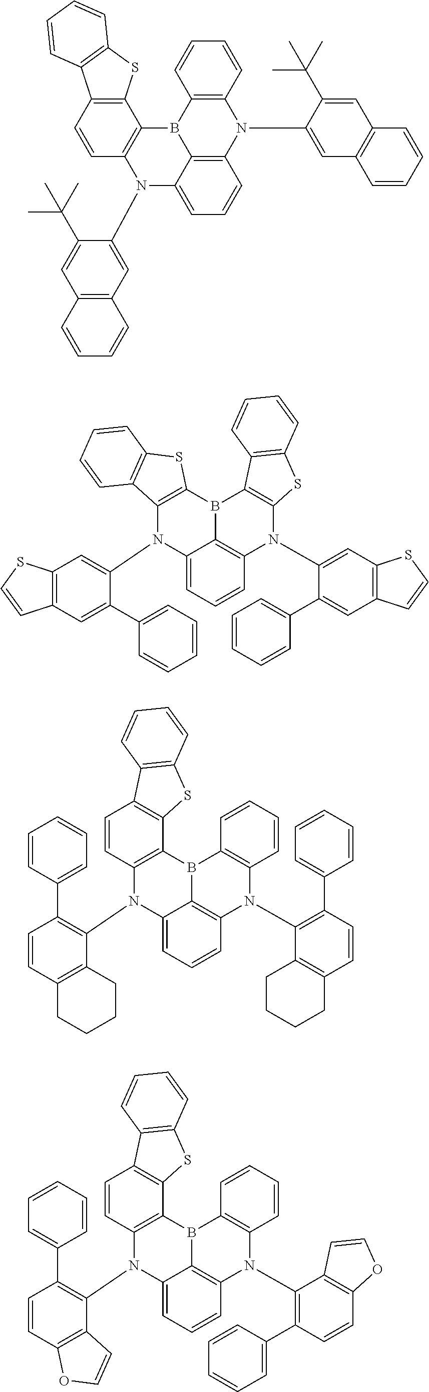

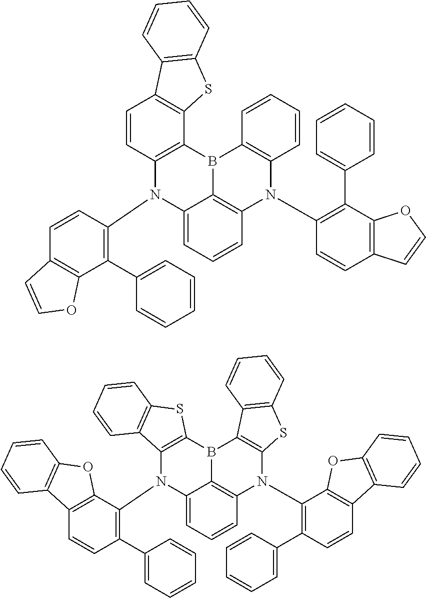 [SMILES: c1ccc(-c2c(N3c4ccccc4B4c5c3cccc5N(c3ccc5ccoc5c3-c3ccccc3)c3ccc5c(sc6ccccc65)c34)ccc3ccoc23)cc1 ; c1ccc(-c2ccc3c(oc4ccccc43)c2N2c3cccc4c3B(c3sc5ccccc5c3N4c3c(-c4ccccc4)ccc4c3oc3ccccc34)c3c2sc2ccccc32)cc1]

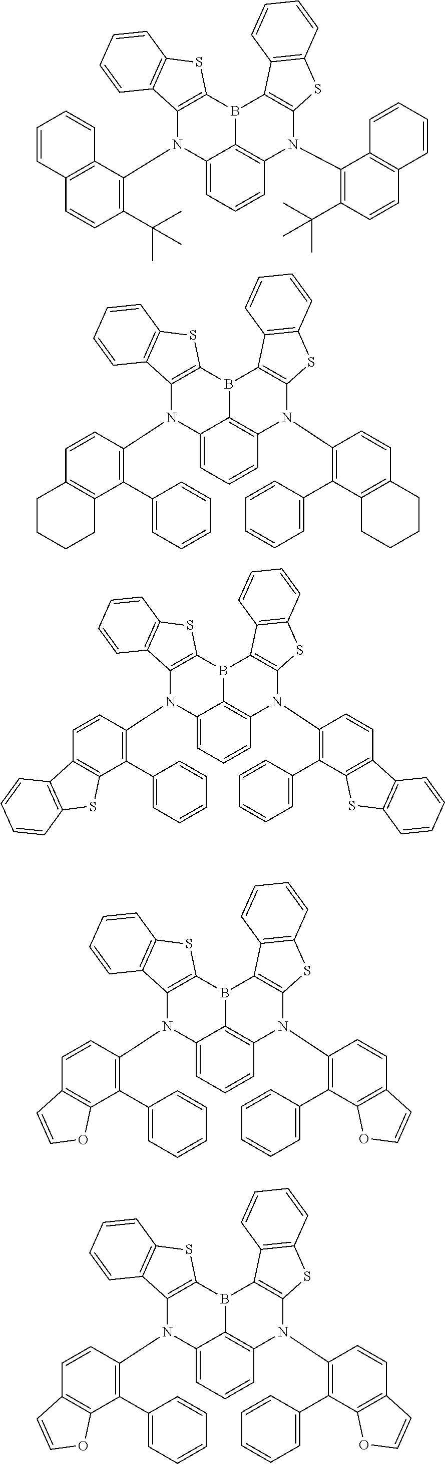

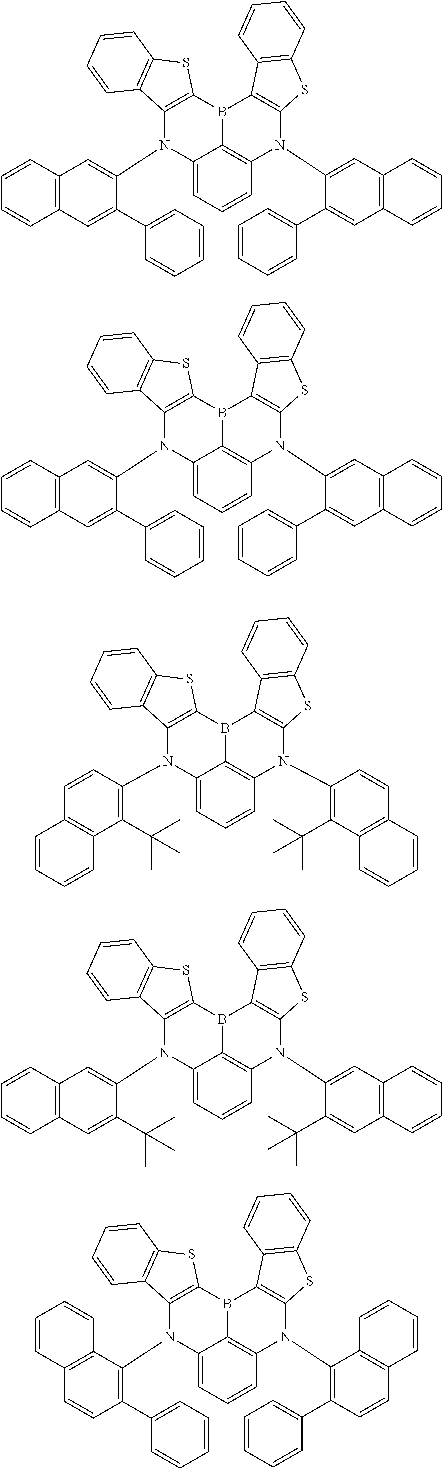

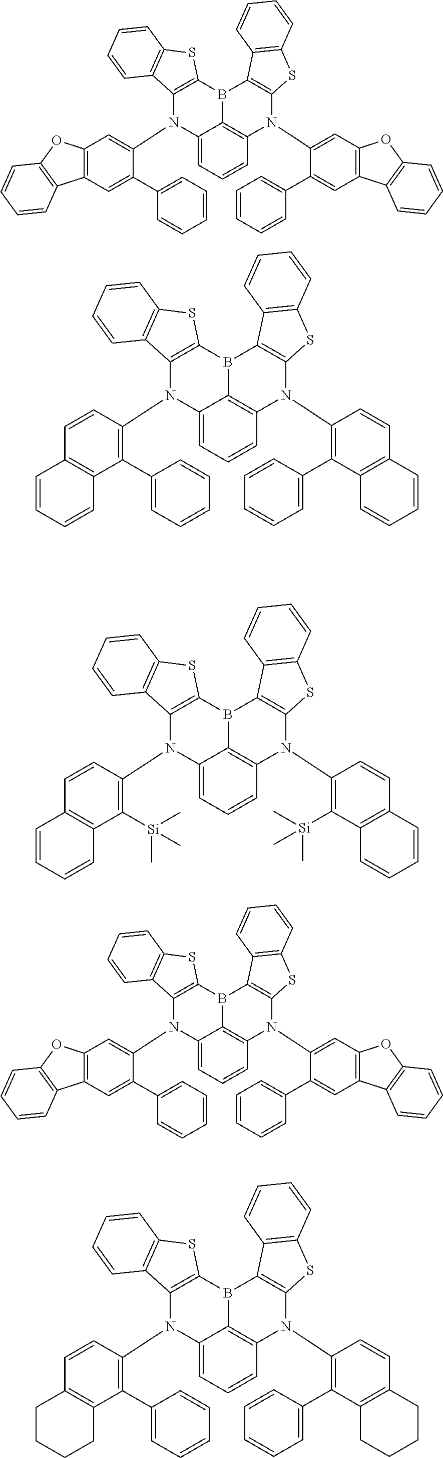

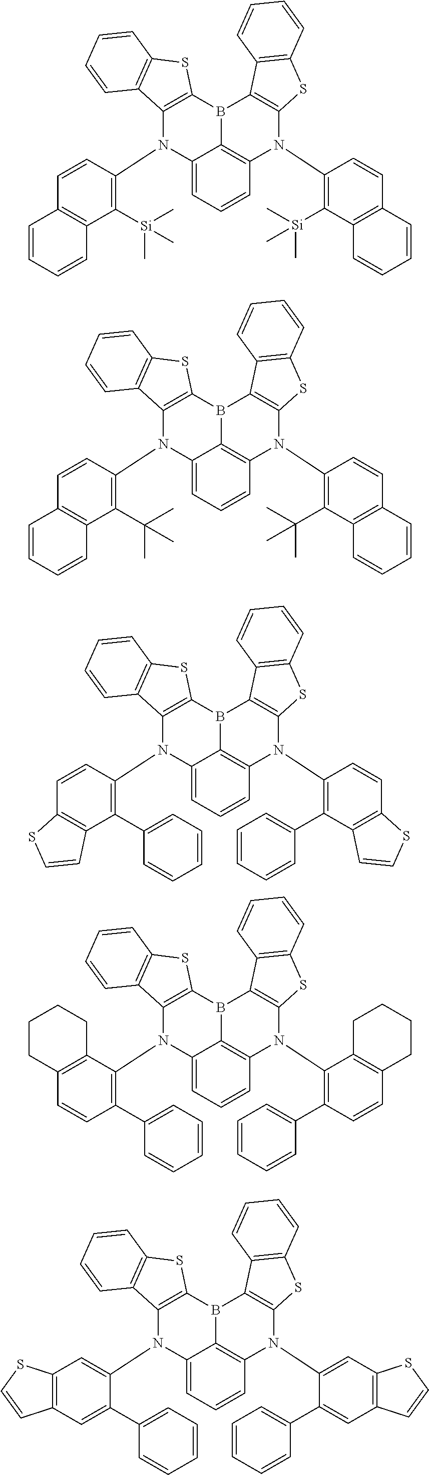

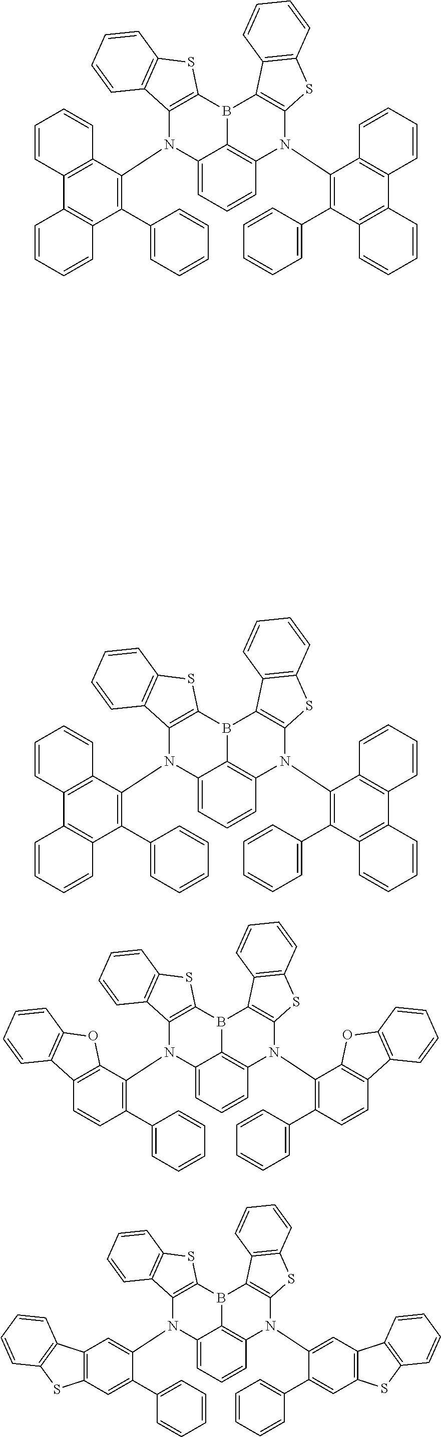

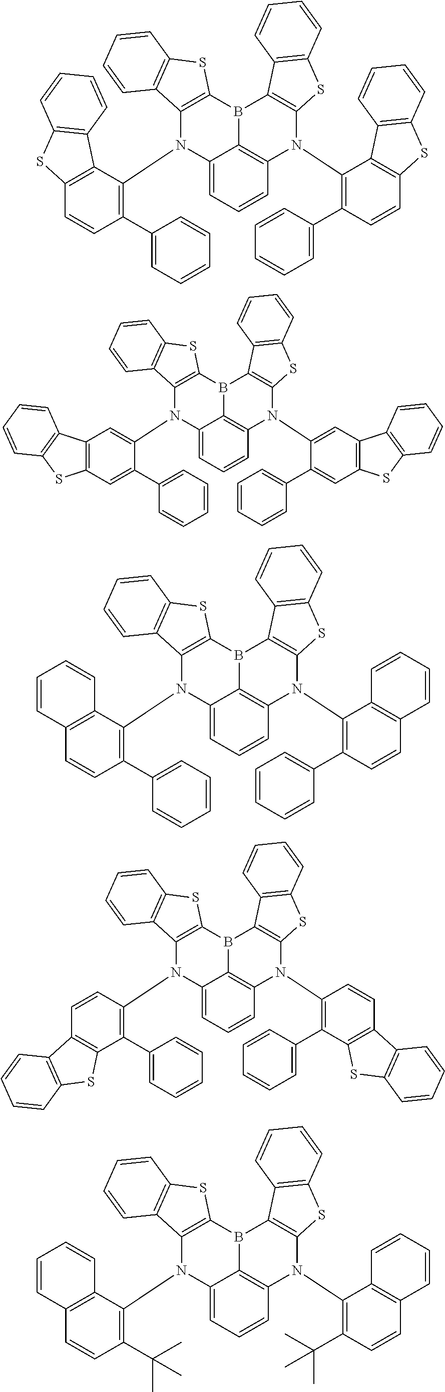

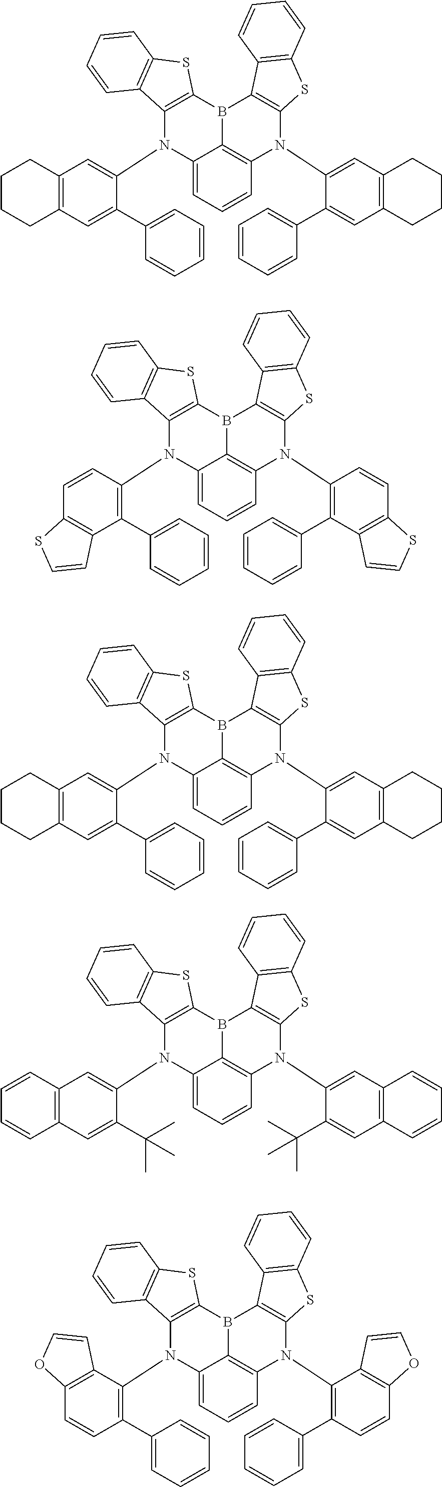

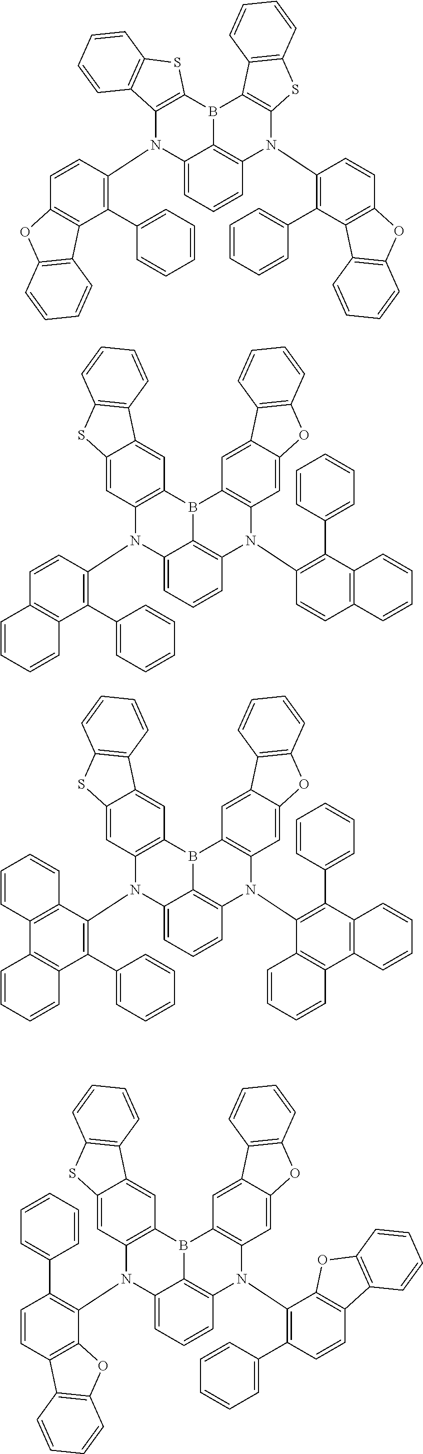

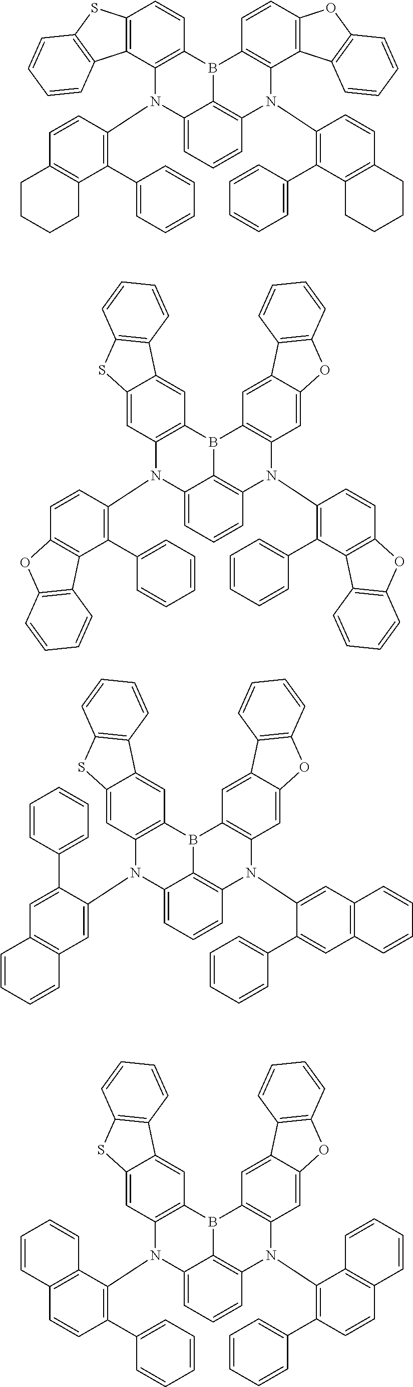

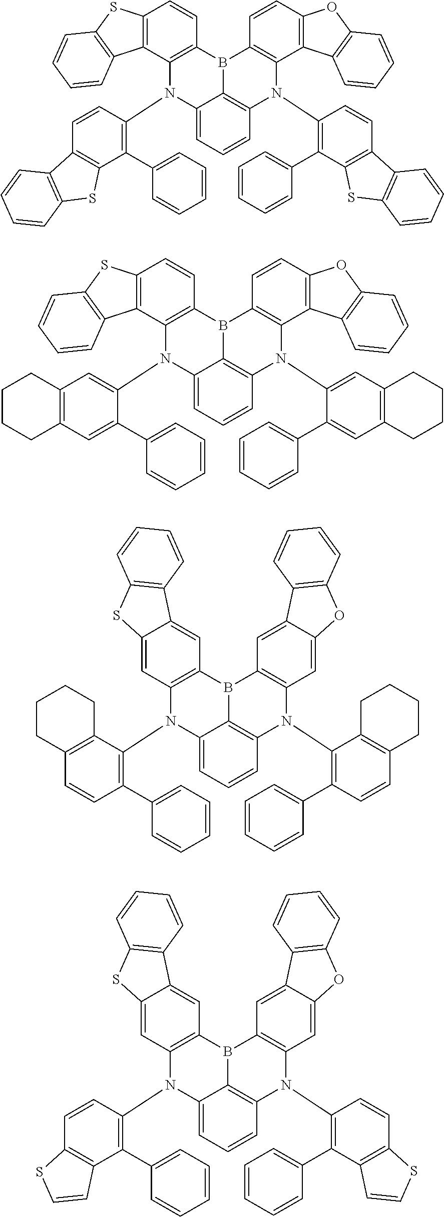

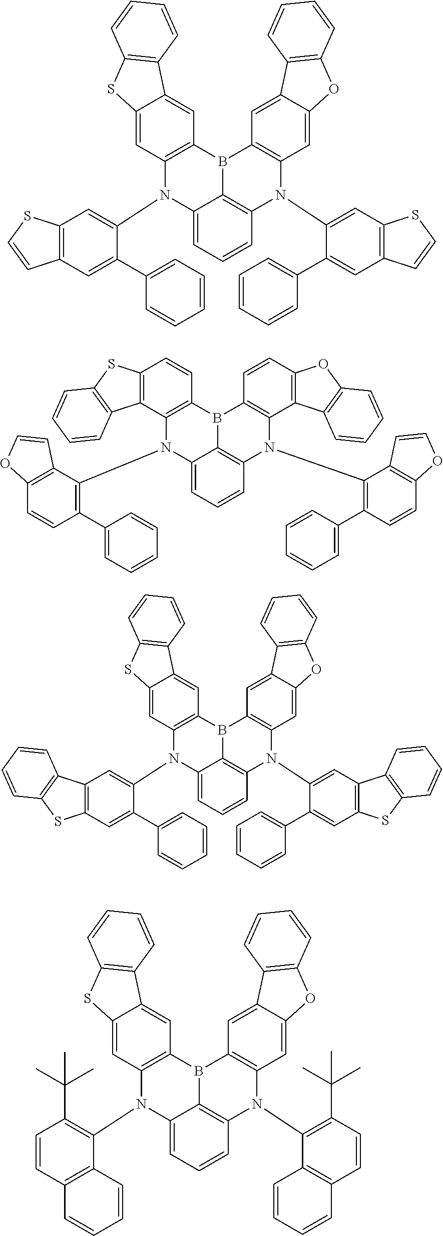

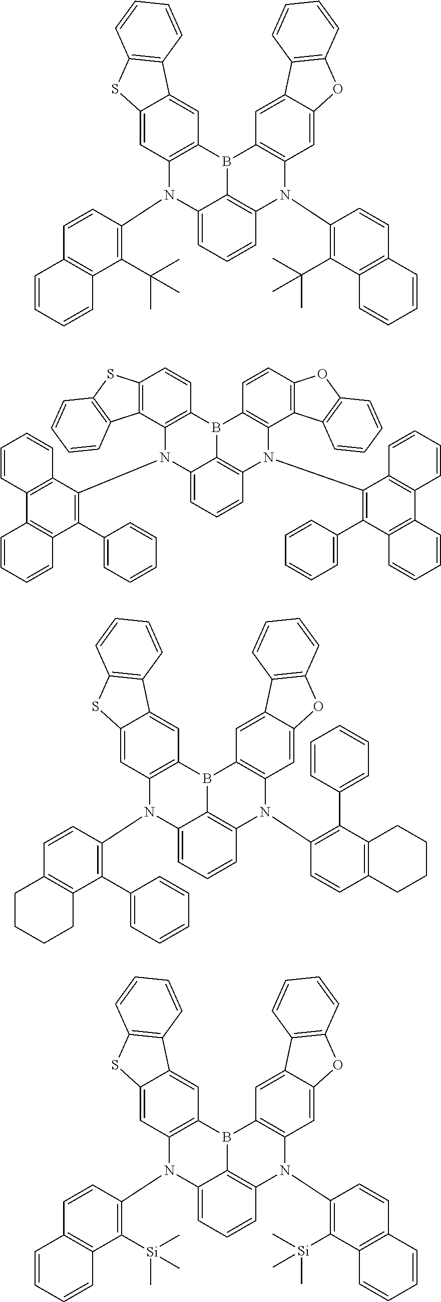

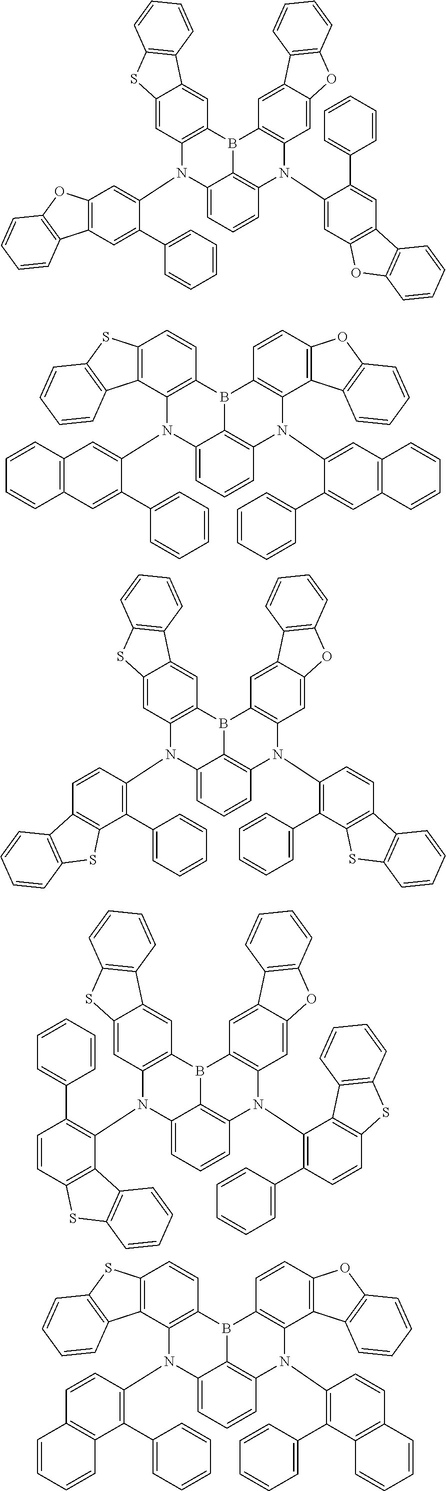

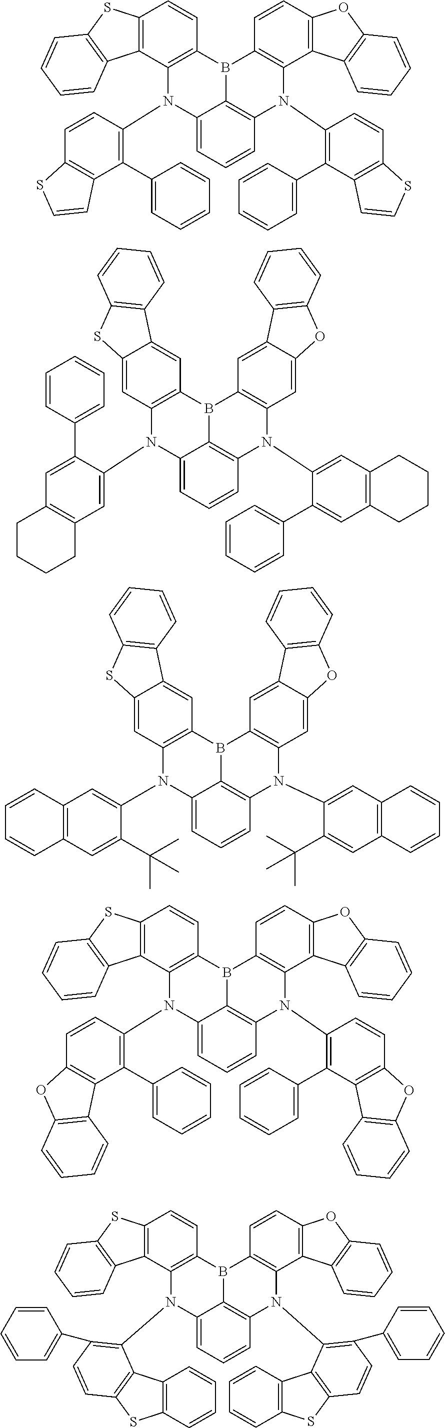

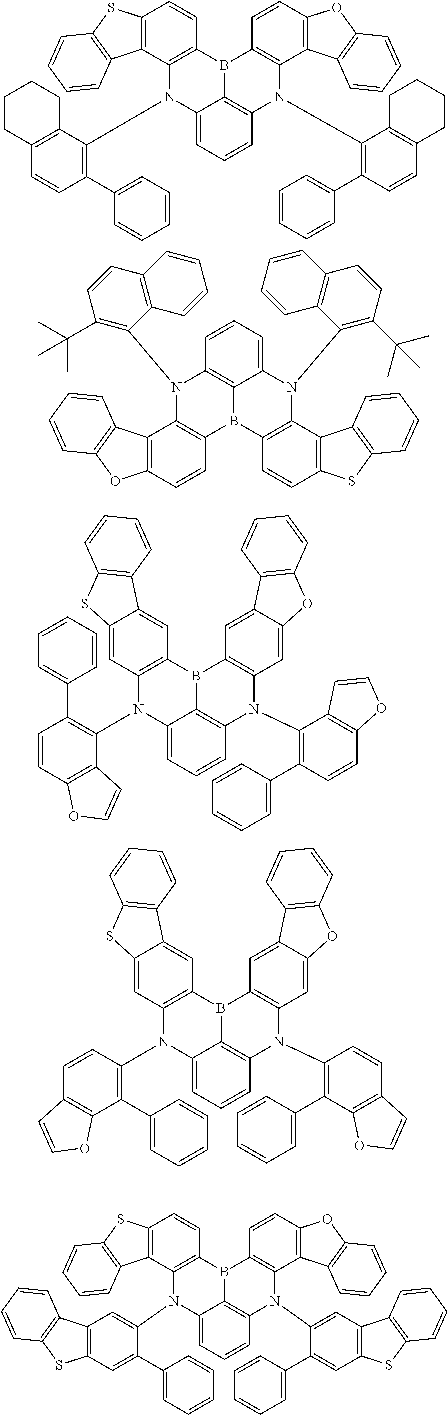

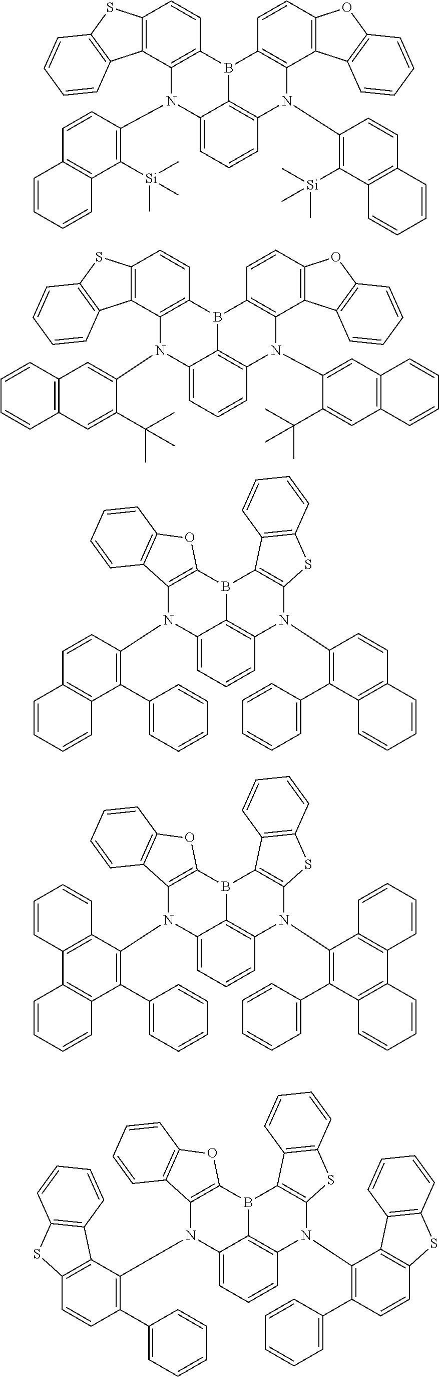

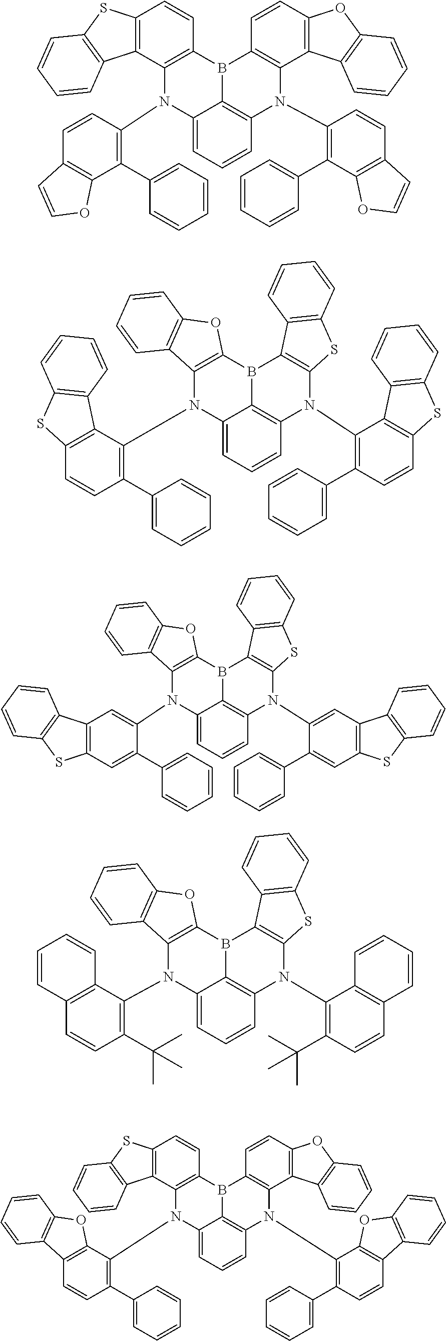

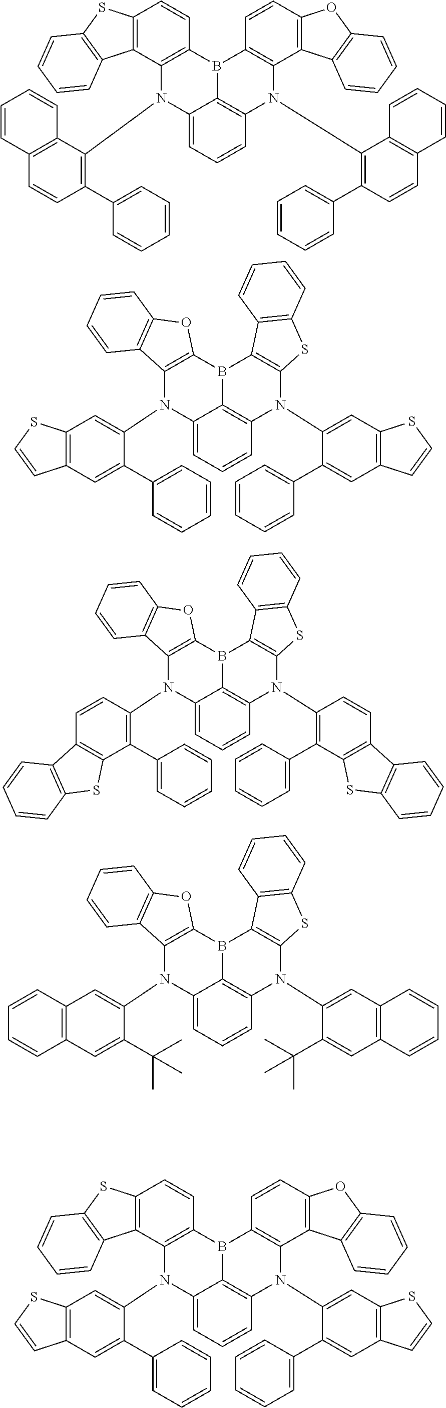

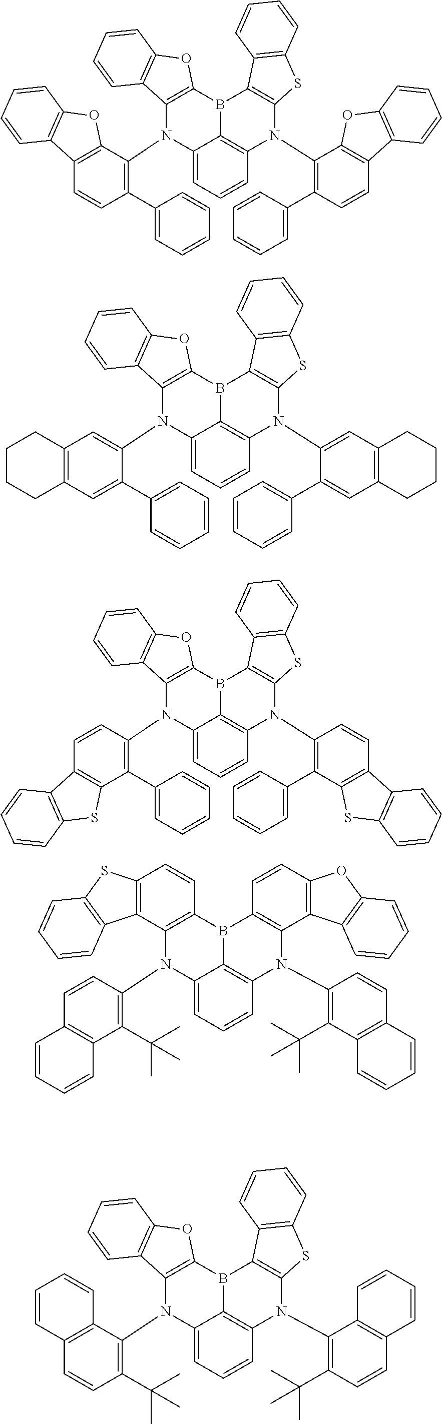

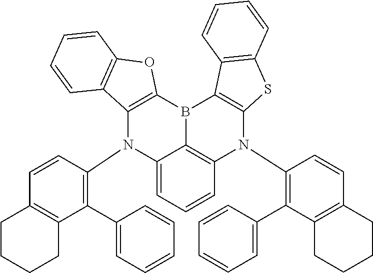 [SMILES: c1ccc(-c2c(N3c4cccc5c4B(c4oc6ccccc6c4N5c4ccc5c(c4-c4ccccc4)CCCC5)c4c3sc3ccccc43)ccc3c2CCCC3)cc1]

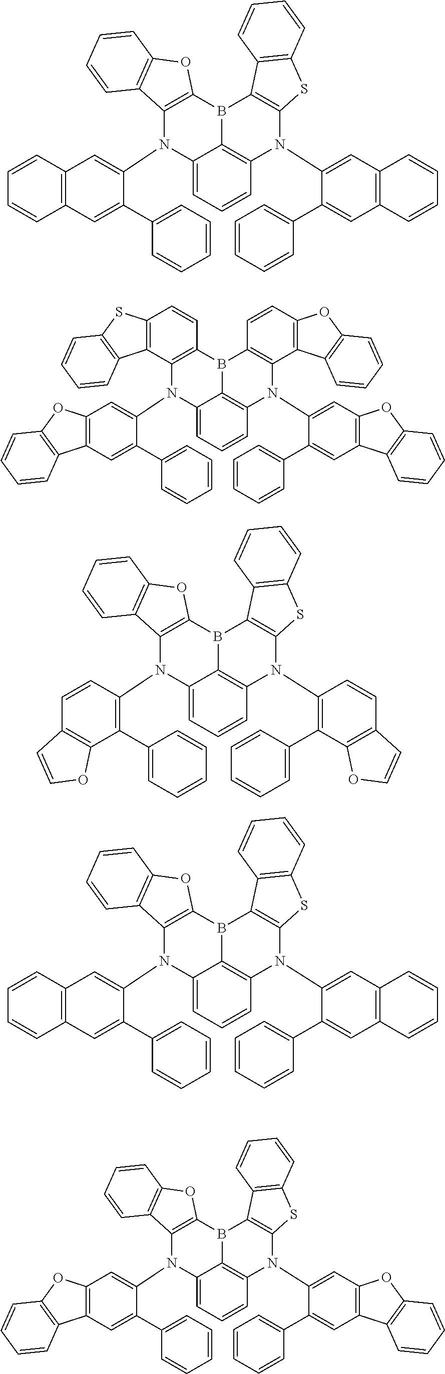

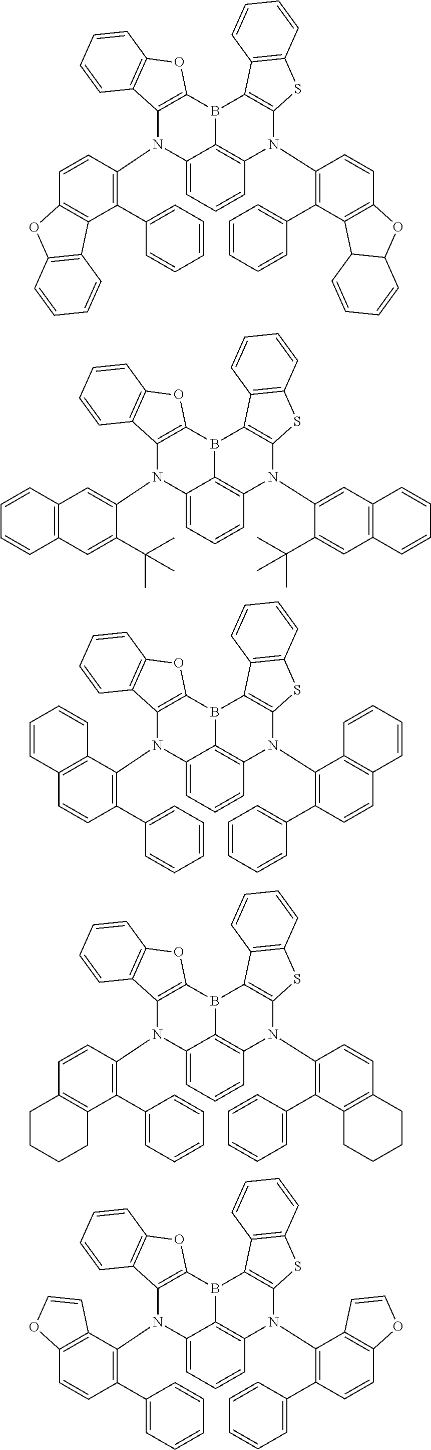

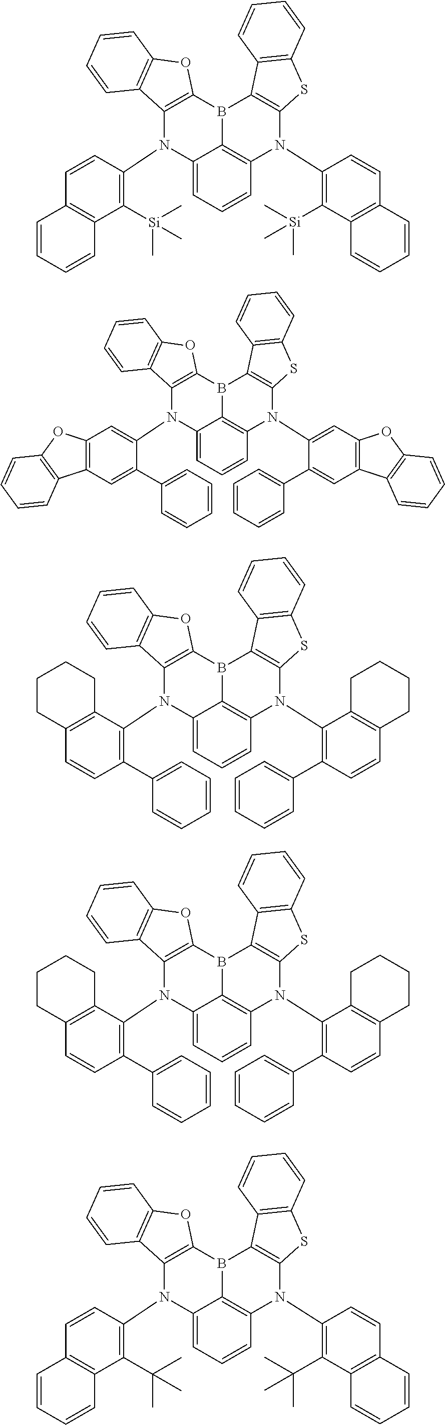

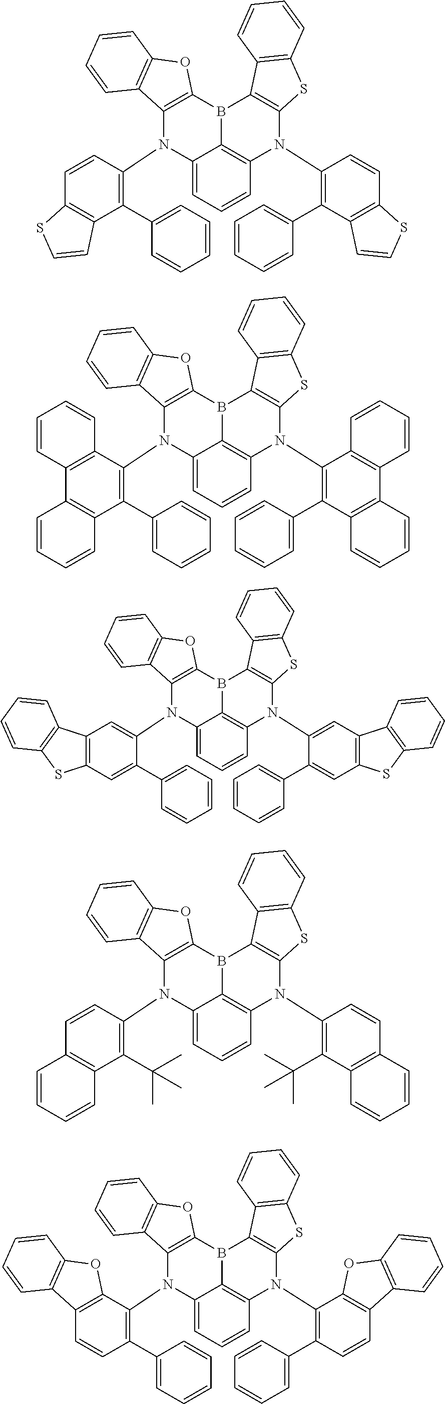

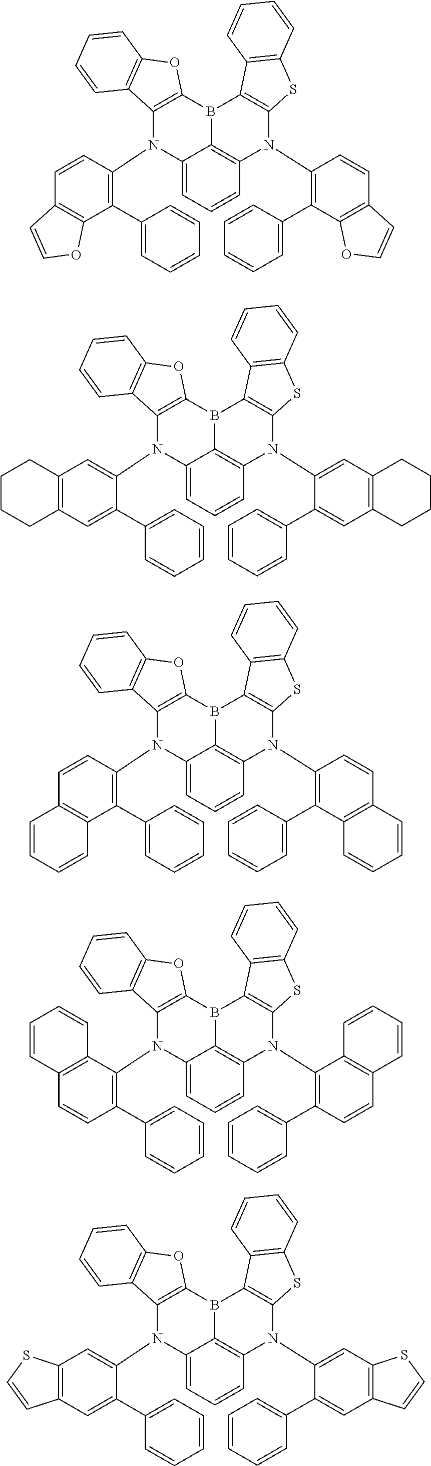

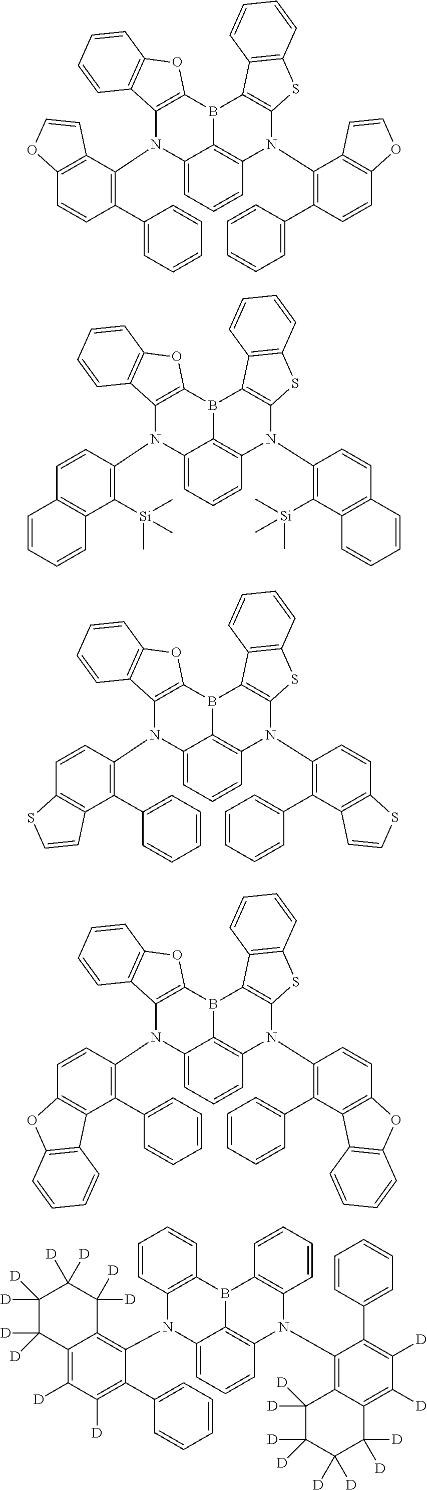

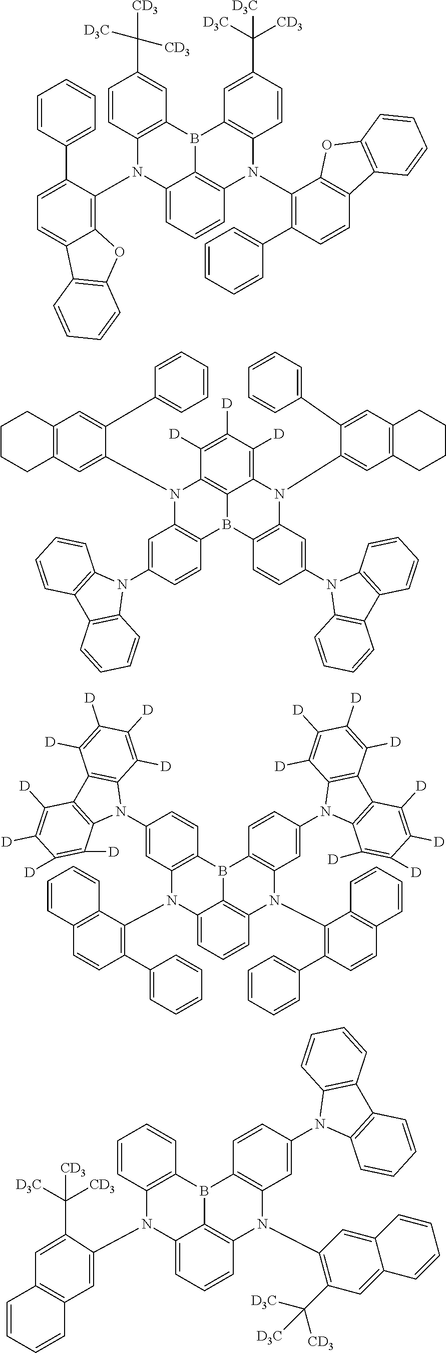

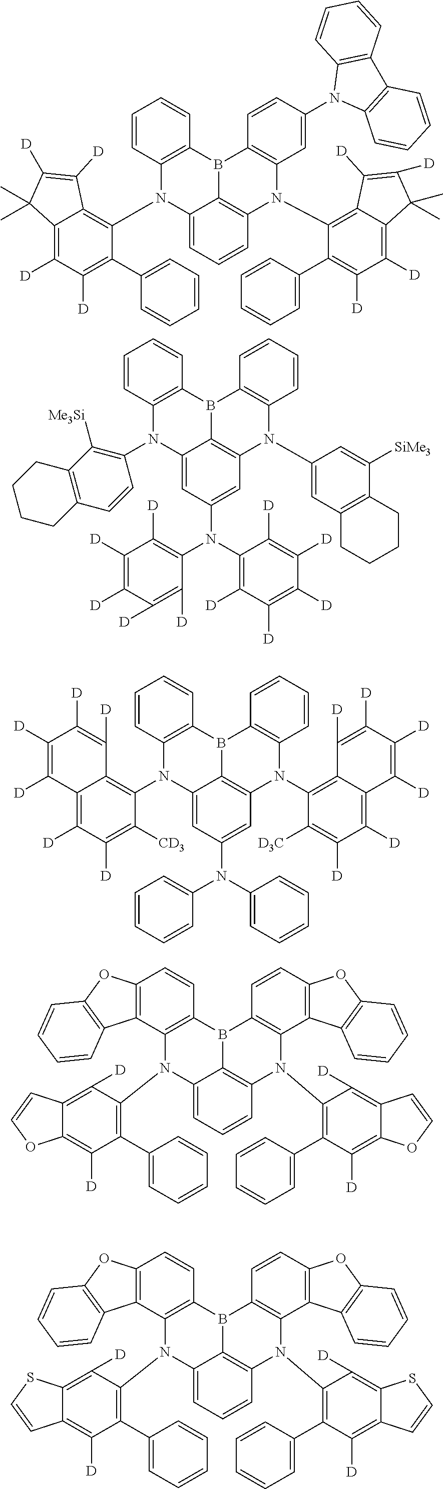

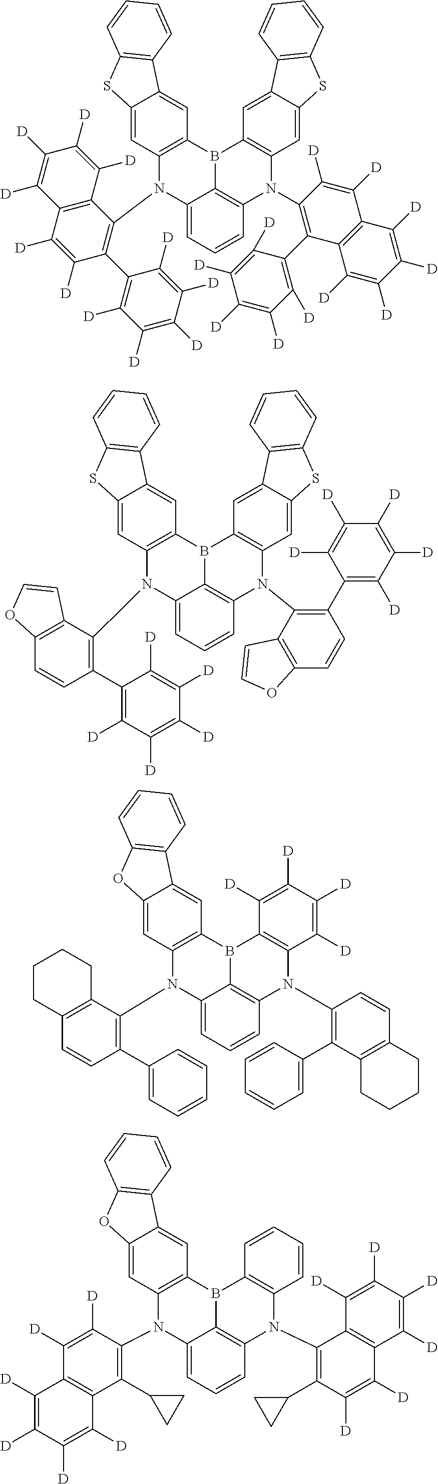

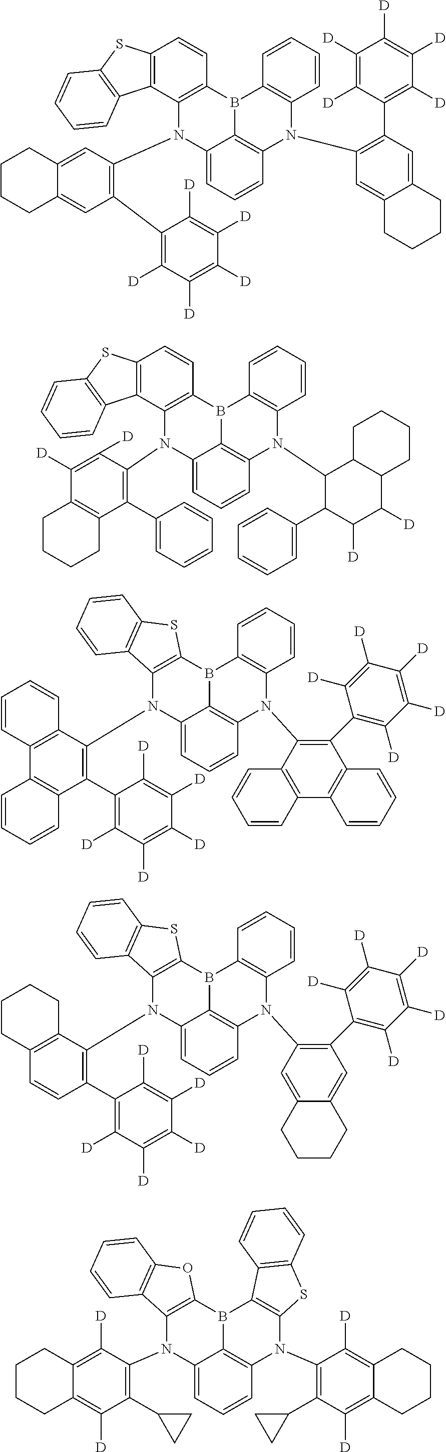

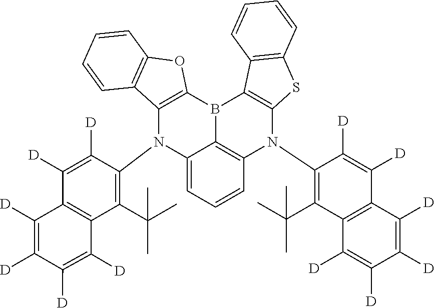 [SMILES: [2H]c1c([2H])c([2H])c2c(C(C)(C)C)c(N3c4cccc5c4B(c4oc6ccccc6c4N5c4c([2H])c([2H])c5c([2H])c([2H])c([2H])c([2H])c5c4C(C)(C)C)c4c3sc3ccccc43)c([2H])c([2H])c2c1[2H]]

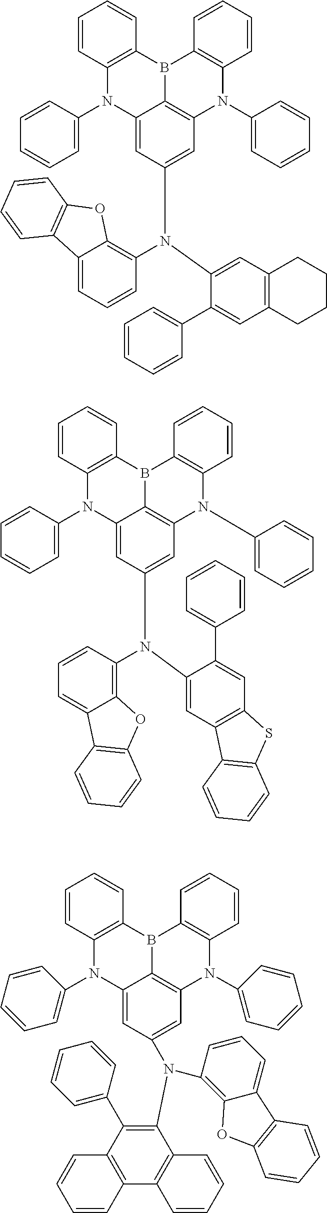 [SMILES: c1ccc(-c2cc3c(cc2N(c2cc4c5c(c2)N(c2ccccc2)c2ccccc2B5c2ccccc2N4c2ccccc2)c2cccc4c2oc2ccccc24)CCCC3)cc1 ; c1ccc(-c2cc3sc4ccccc4c3cc2N(c2cc3c4c(c2)N(c2ccccc2)c2ccccc2B4c2ccccc2N3c2ccccc2)c2cccc3c2oc2ccccc23)cc1 ; c1ccc(-c2c(N(c3cc4c5c(c3)N(c3ccccc3)c3ccccc3B5c3ccccc3N4c3ccccc3)c3cccc4c3oc3ccccc34)c3ccccc3c3ccccc23)cc1]

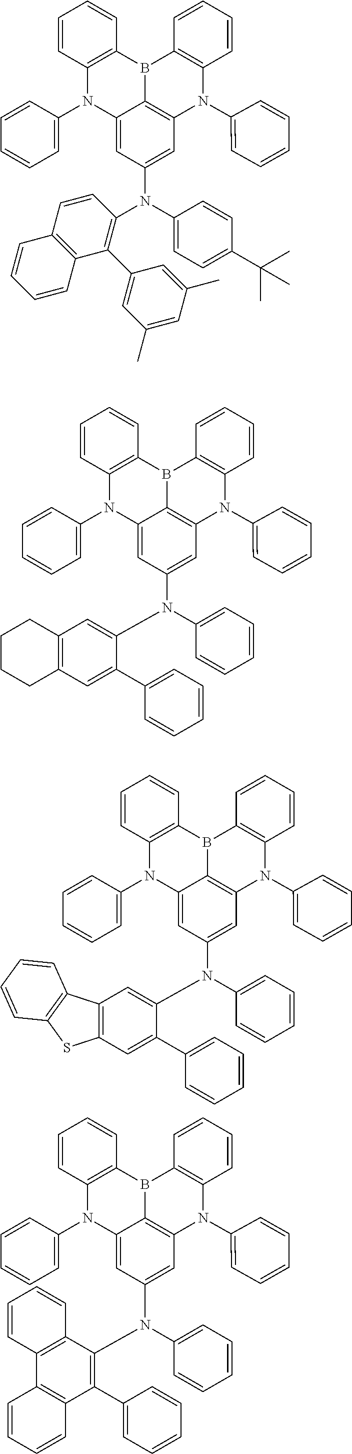

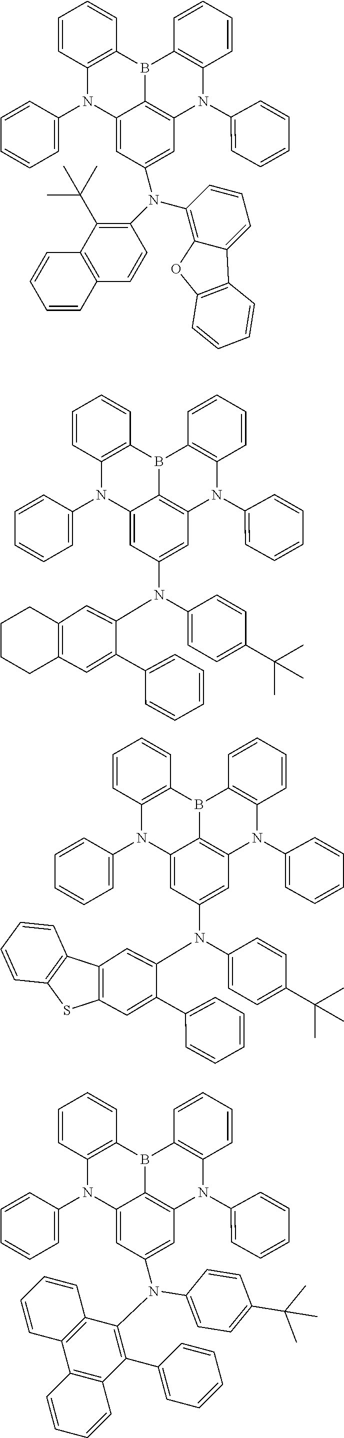

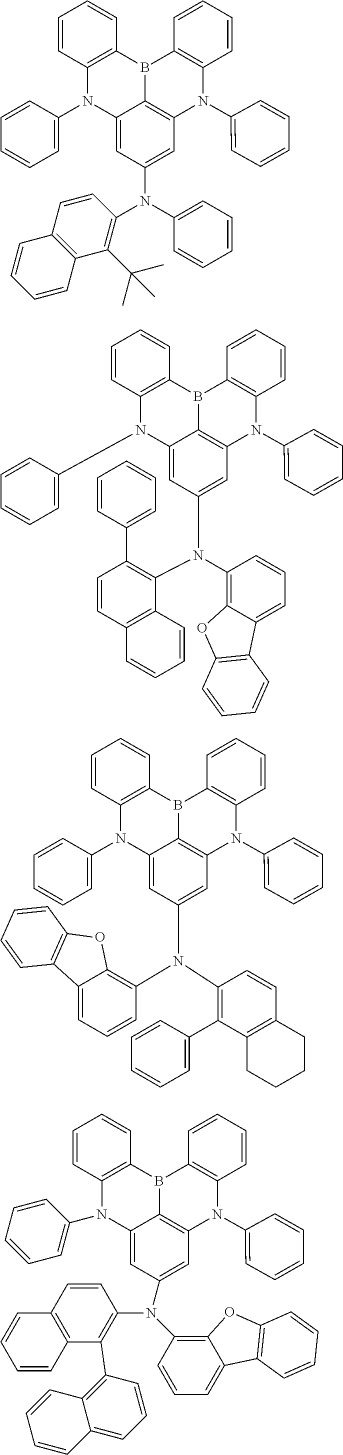

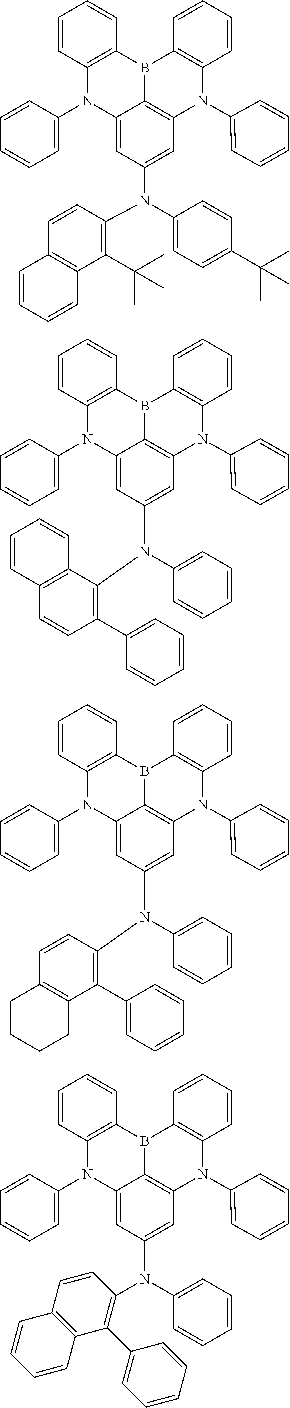

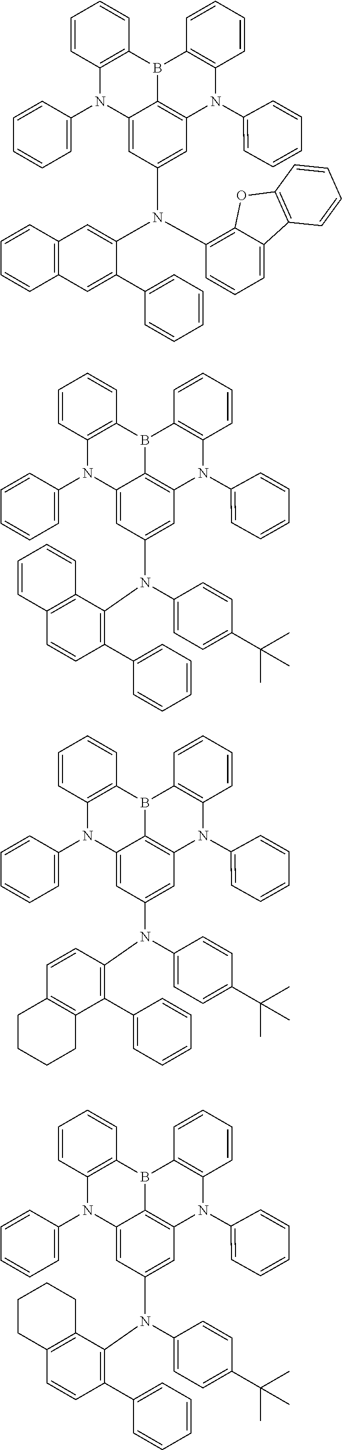

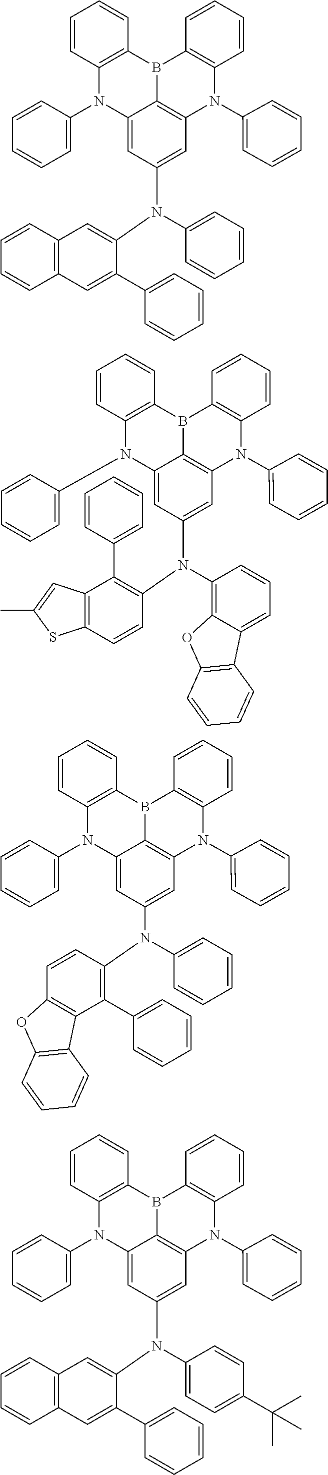

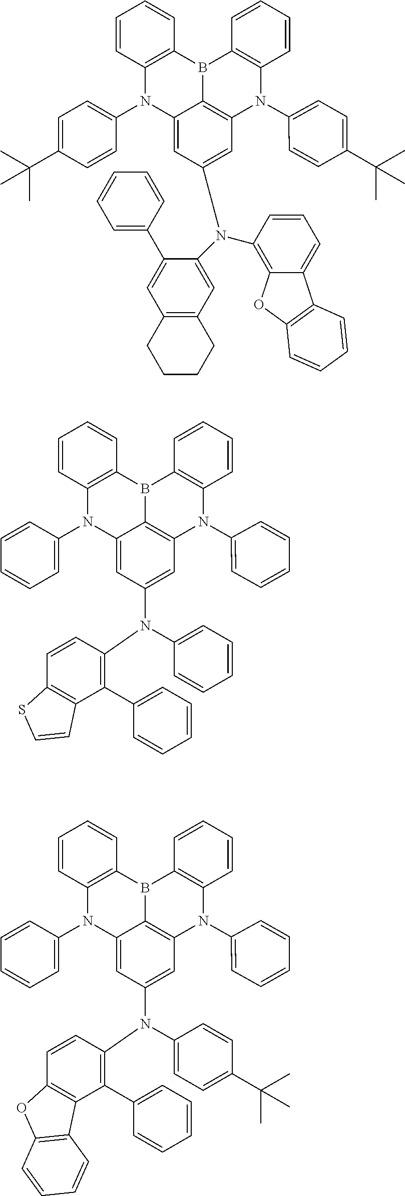 [SMILES: CC(C)(C)c1ccc(N2c3ccccc3B3c4ccccc4N(c4ccc(C(C)(C)C)cc4)c4cc(N(c5cc6c(cc5-c5ccccc5)CCCC6)c5cccc6c5oc5ccccc56)cc2c43)cc1 ; c1ccc(-c2c(N(c3ccccc3)c3cc4c5c(c3)N(c3ccccc3)c3ccccc3B5c3ccccc3N4c3ccccc3)ccc3sccc23)cc1 ; CC(C)(C)c1ccc(N(c2cc3c4c(c2)N(c2ccccc2)c2ccccc2B4c2ccccc2N3c2ccccc2)c2ccc3oc4ccccc4c3c2-c2ccccc2)cc1]

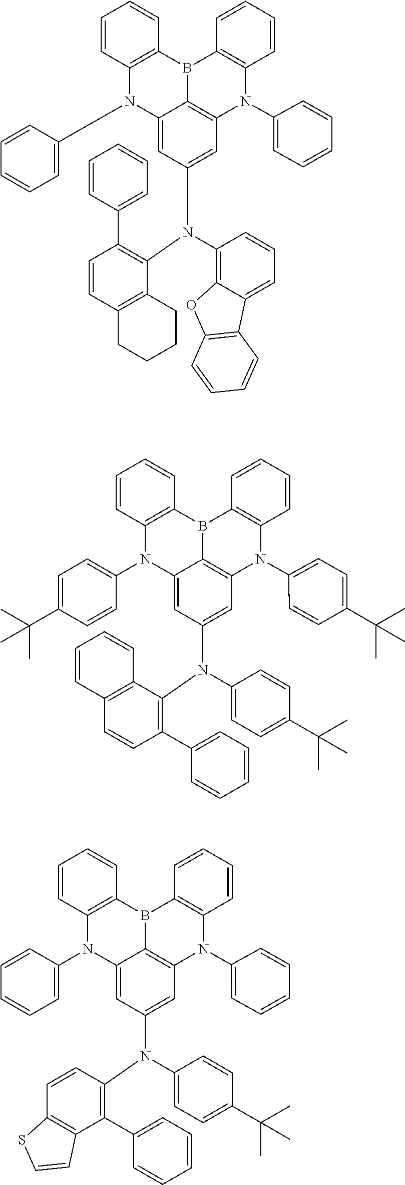 [SMILES: c1ccc(-c2ccc3c(c2N(c2cc4c5c(c2)N(c2ccccc2)c2ccccc2B5c2ccccc2N4c2ccccc2)c2cccc4c2oc2ccccc24)CCCC3)cc1 ; CC(C)(C)c1ccc(N2c3ccccc3B3c4ccccc4N(c4ccc(C(C)(C)C)cc4)c4cc(N(c5ccc(C(C)(C)C)cc5)c5c(-c6ccccc6)ccc6ccccc56)cc2c43)cc1 ; CC(C)(C)c1ccc(N(c2cc3c4c(c2)N(c2ccccc2)c2ccccc2B4c2ccccc2N3c2ccccc2)c2ccc3sccc3c2-c2ccccc2)cc1]

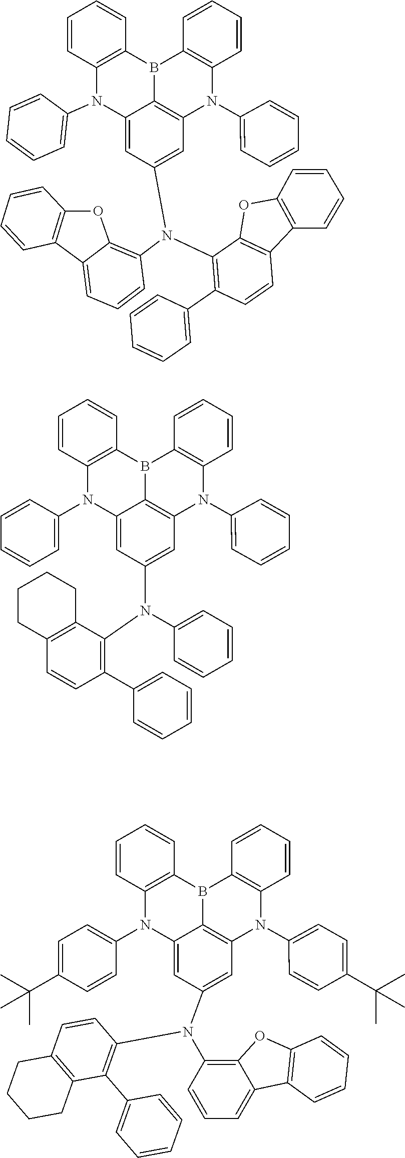 [SMILES: c1ccc(-c2ccc3c(oc4ccccc43)c2N(c2cc3c4c(c2)N(c2ccccc2)c2ccccc2B4c2ccccc2N3c2ccccc2)c2cccc3c2oc2ccccc23)cc1 ; c1ccc(-c2ccc3c(c2N(c2ccccc2)c2cc4c5c(c2)N(c2ccccc2)c2ccccc2B5c2ccccc2N4c2ccccc2)CCCC3)cc1 ; CC(C)(C)c1ccc(N2c3ccccc3B3c4ccccc4N(c4ccc(C(C)(C)C)cc4)c4cc(N(c5ccc6c(c5-c5ccccc5)CCCC6)c5cccc6c5oc5ccccc56)cc2c43)cc1]

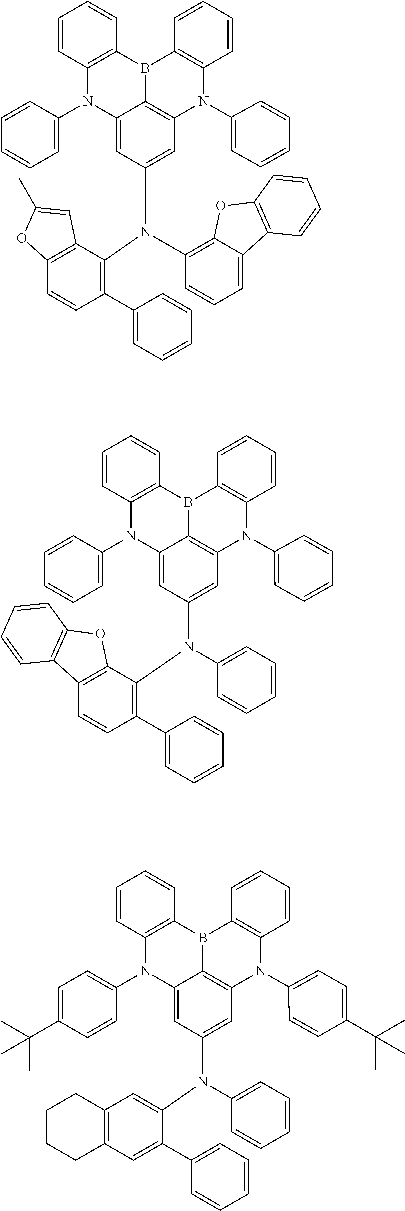 [SMILES: Cc1cc2c(N(c3cc4c5c(c3)N(c3ccccc3)c3ccccc3B5c3ccccc3N4c3ccccc3)c3cccc4c3oc3ccccc34)c(-c3ccccc3)ccc2o1 ; c1ccc(-c2ccc3c(oc4ccccc43)c2N(c2ccccc2)c2cc3c4c(c2)N(c2ccccc2)c2ccccc2B4c2ccccc2N3c2ccccc2)cc1 ; CC(C)(C)c1ccc(N2c3ccccc3B3c4ccccc4N(c4ccc(C(C)(C)C)cc4)c4cc(N(c5ccccc5)c5cc6c(cc5-c5ccccc5)CCCC6)cc2c43)cc1]

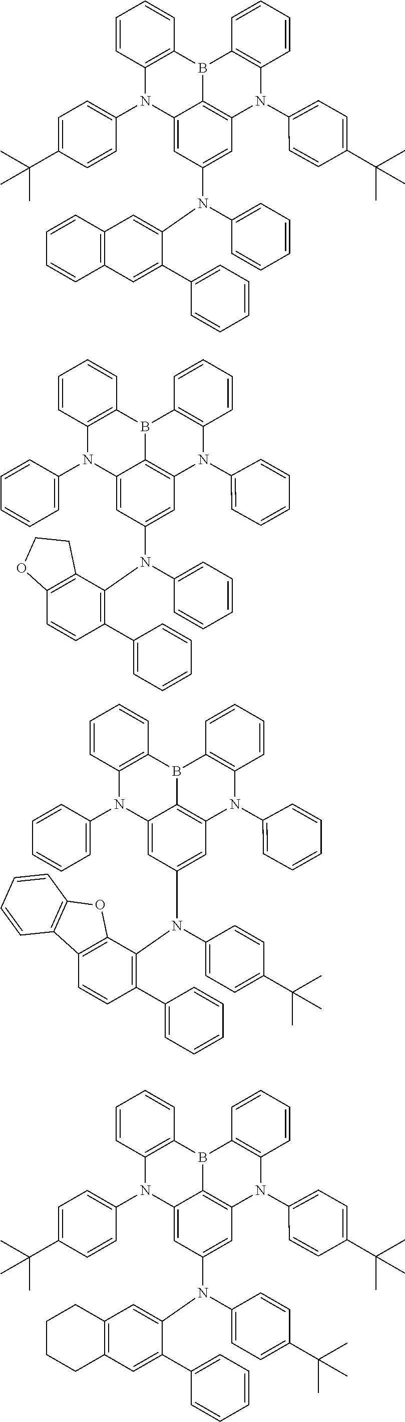

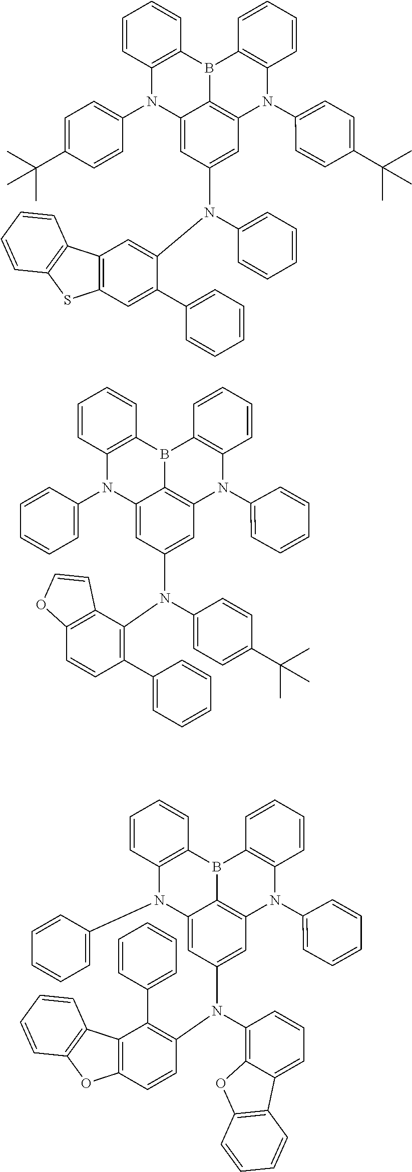

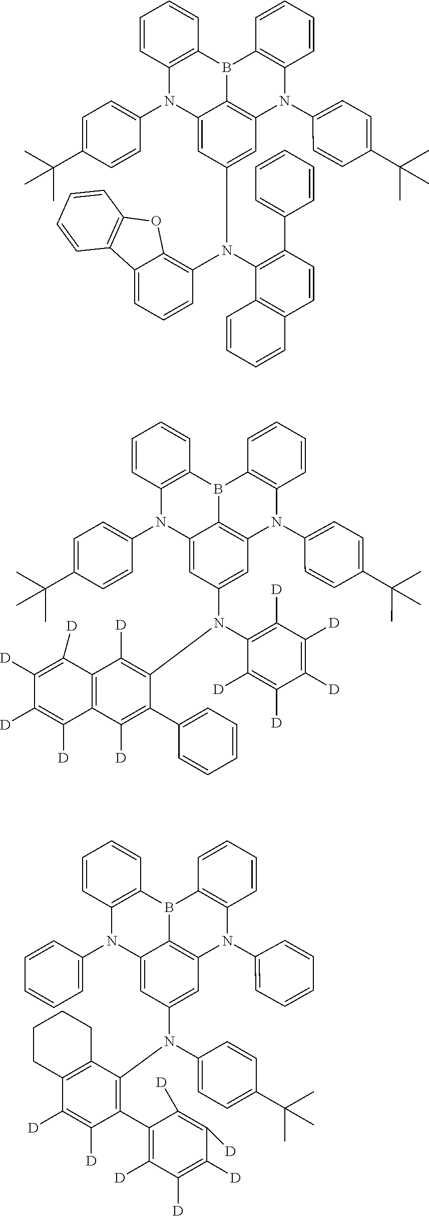 [SMILES: CC(C)(C)c1ccc(N2c3ccccc3B3c4ccccc4N(c4ccc(C(C)(C)C)cc4)c4cc(N(c5c(-c6ccccc6)ccc6ccccc56)c5cccc6c5oc5ccccc56)cc2c43)cc1 ; [2H]c1c([2H])c([2H])c(N(c2cc3c4c(c2)N(c2ccc(C(C)(C)C)cc2)c2ccccc2B4c2ccccc2N3c2ccc(C(C)(C)C)cc2)c2c(-c3ccccc3)c([2H])c3c([2H])c([2H])c([2H])c([2H])c3c2[2H])c([2H])c1[2H] ; [2H]c1c([2H])c([2H])c(-c2c([2H])c([2H])c3c(c2N(c2ccc(C(C)(C)C)cc2)c2cc4c5c(c2)N(c2ccccc2)c2ccccc2B5c2ccccc2N4c2ccccc2)CCCC3)c([2H])c1[2H]]

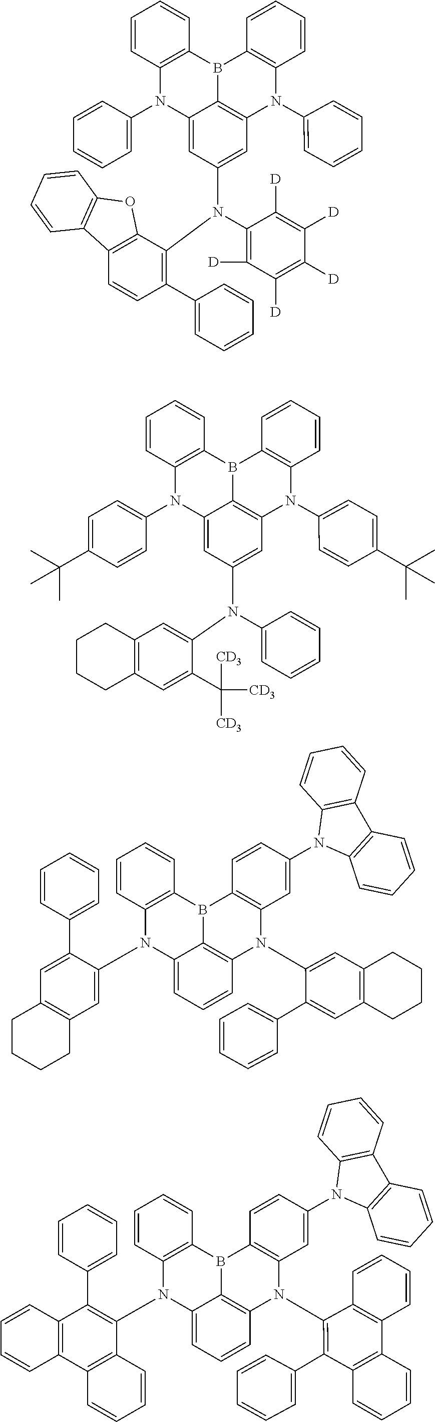

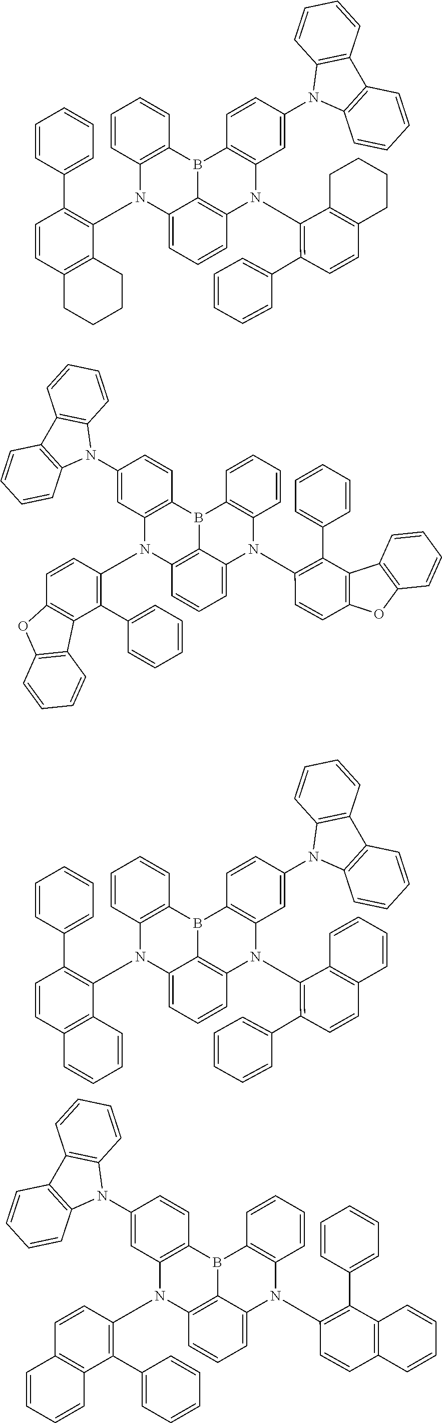

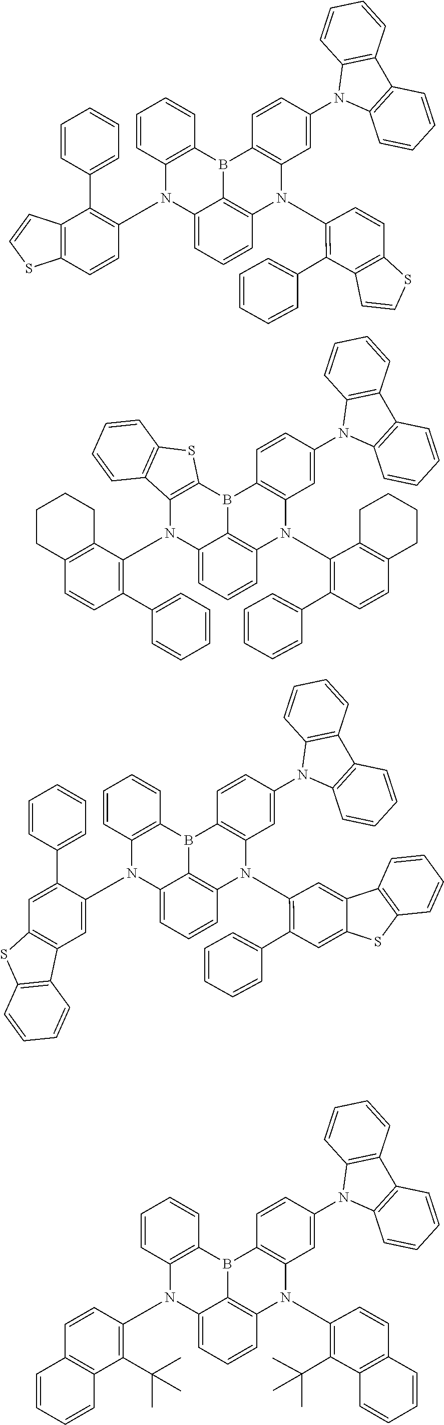

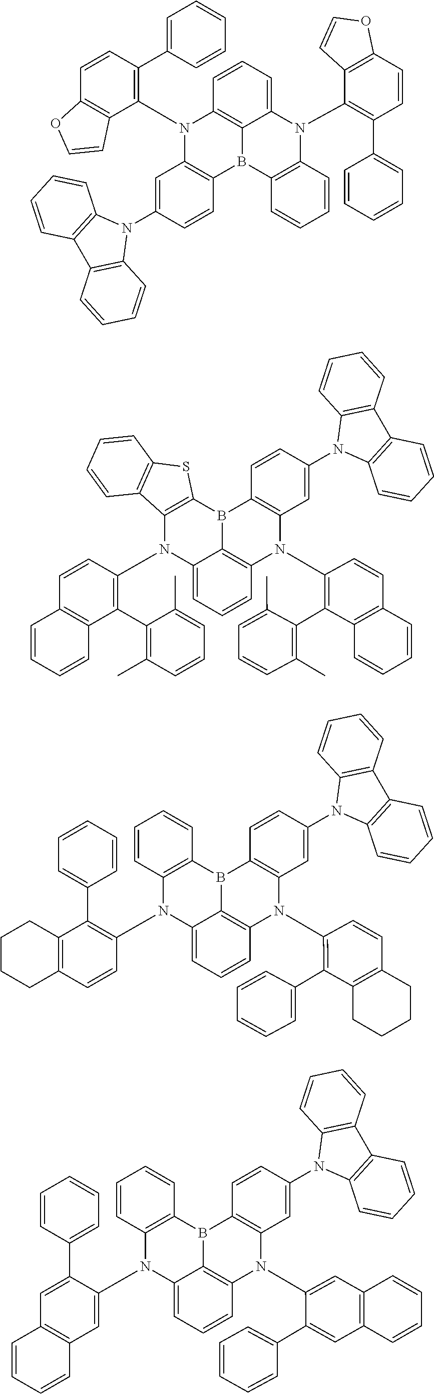

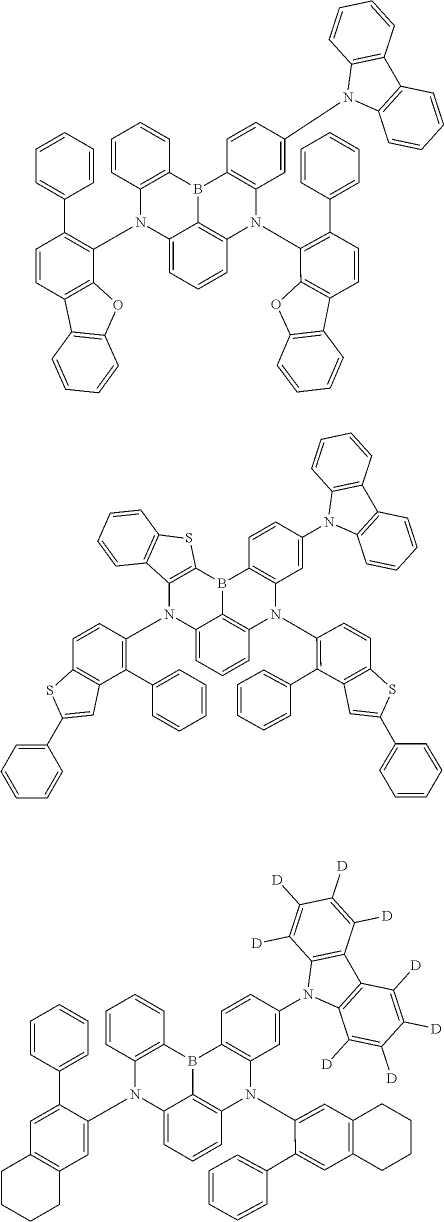 [SMILES: c1ccc(-c2ccc3c(oc4ccccc43)c2N2c3ccccc3B3c4ccc(-n5c6ccccc6c6ccccc65)cc4N(c4c(-c5ccccc5)ccc5c4oc4ccccc45)c4cccc2c43)cc1 ; c1ccc(-c2cc3c(-c4ccccc4)c(N4c5cc(-n6c7ccccc7c7ccccc76)ccc5B5c6sc7ccccc7c6N(c6ccc7sc(-c8ccccc8)cc7c6-c6ccccc6)c6cccc4c65)ccc3s2)cc1 ; [2H]c1c([2H])c([2H])c2c(c1[2H])c1c([2H])c([2H])c([2H])c([2H])c1n2-c1ccc2c(c1)N(c1cc3c(cc1-c1ccccc1)CCCC3)c1cccc3c1B2c1ccccc1N3c1cc2c(cc1-c1ccccc1)CCCC2]

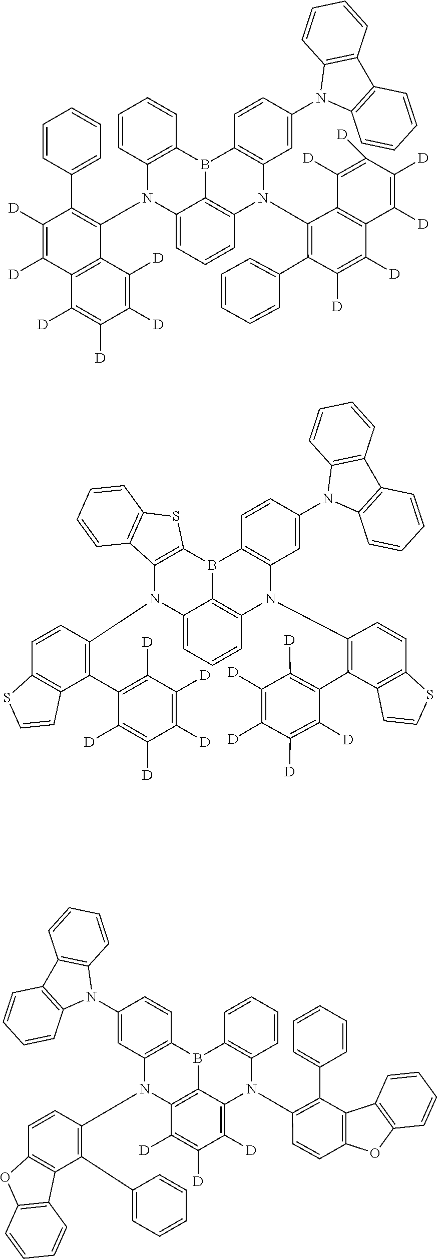 [SMILES: [2H]c1c([2H])c([2H])c2c(N3c4ccccc4B4c5ccc(-n6c7ccccc7c7ccccc76)cc5N(c5c(-c6ccccc6)c([2H])c([2H])c6c([2H])c([2H])c([2H])c([2H])c56)c5cccc3c54)c(-c3ccccc3)c([2H])c([2H])c2c1[2H] ; [2H]c1c([2H])c([2H])c(-c2c(N3c4cc(-n5c6ccccc6c6ccccc65)ccc4B4c5sc6ccccc6c5N(c5ccc6sccc6c5-c5c([2H])c([2H])c([2H])c([2H])c5[2H])c5cccc3c54)ccc3sccc23)c([2H])c1[2H] ; [2H]c1c([2H])c2c3c(c1[2H])N(c1ccc4oc5ccccc5c4c1-c1ccccc1)c1cc(-n4c5ccccc5c5ccccc54)ccc1B3c1ccccc1N2c1ccc2oc3ccccc3c2c1-c1ccccc1]

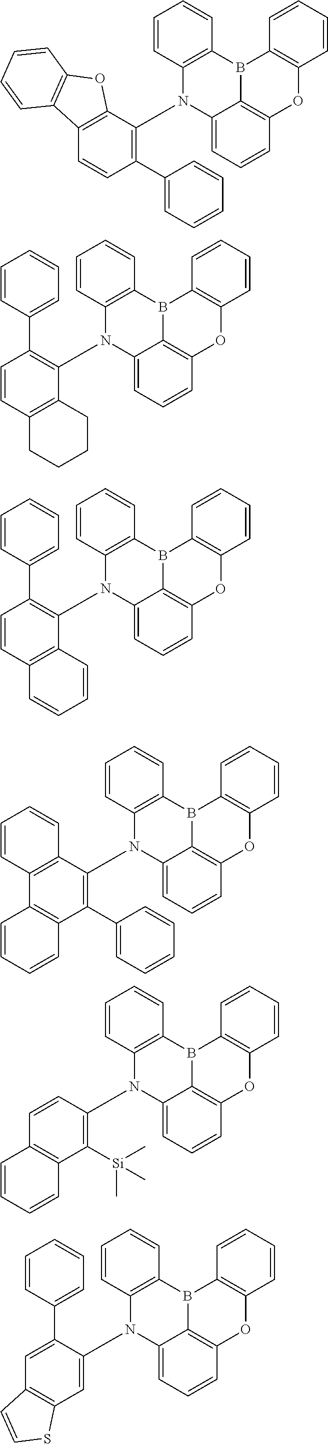

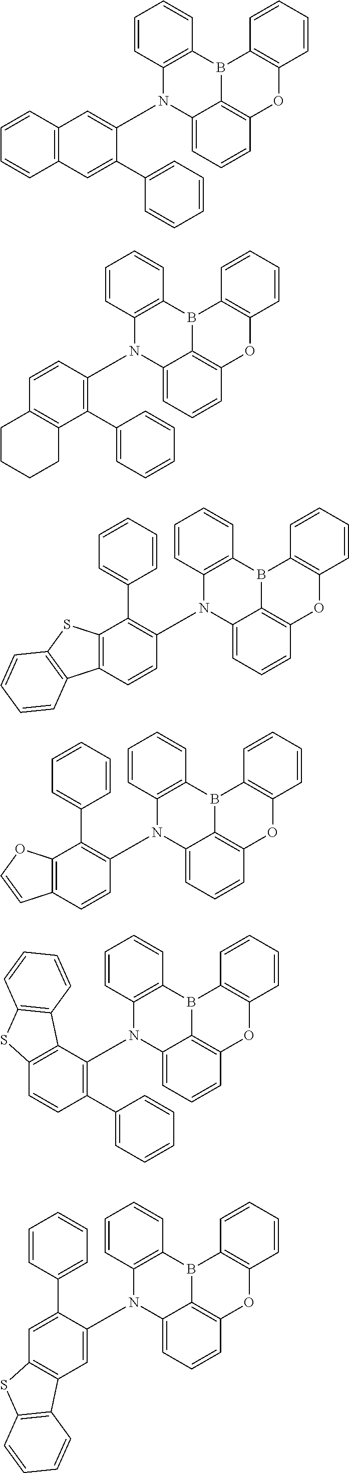

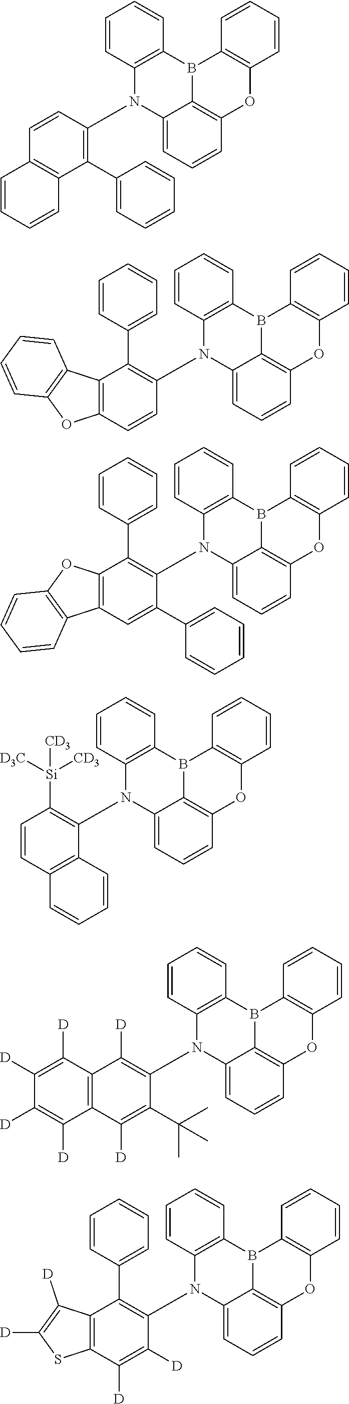

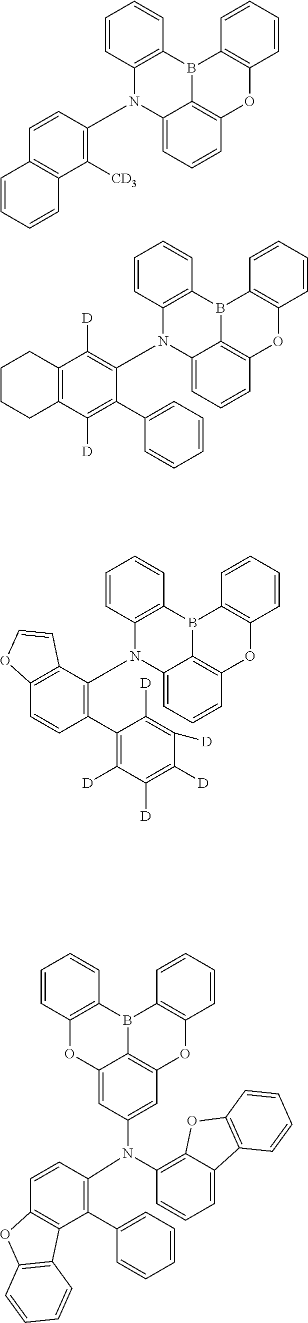 [SMILES: [2H]C([2H])([2H])c1c(N2c3ccccc3B3c4ccccc4Oc4cccc2c43)ccc2ccccc12 ; [2H]c1c2c(c([2H])c(N3c4ccccc4B4c5ccccc5Oc5cccc3c54)c1-c1ccccc1)CCCC2 ; [2H]c1c([2H])c([2H])c(-c2ccc3occc3c2N2c3ccccc3B3c4ccccc4Oc4cccc2c43)c([2H])c1[2H] ; c1ccc(-c2c(N(c3cc4c5c(c3)Oc3ccccc3B5c3ccccc3O4)c3cccc4c3oc3ccccc34)ccc3oc4ccccc4c23)cc1]

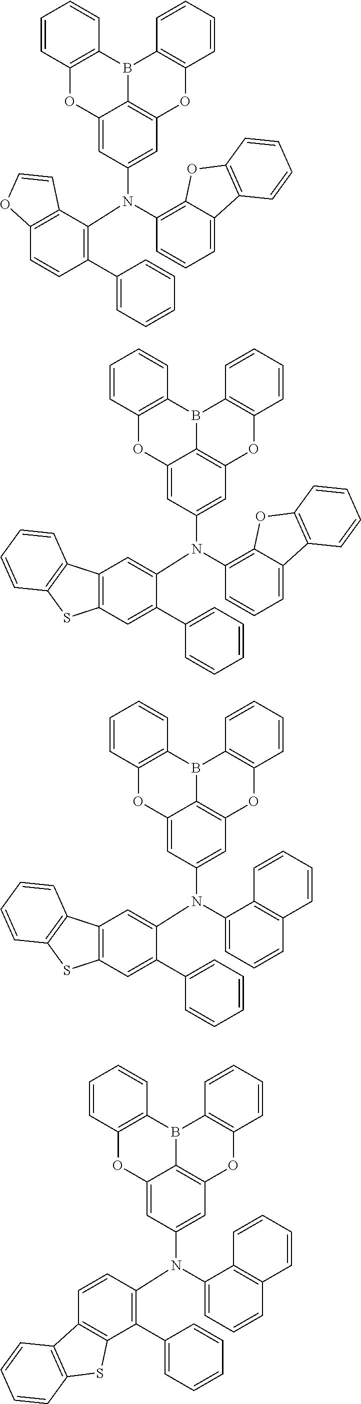

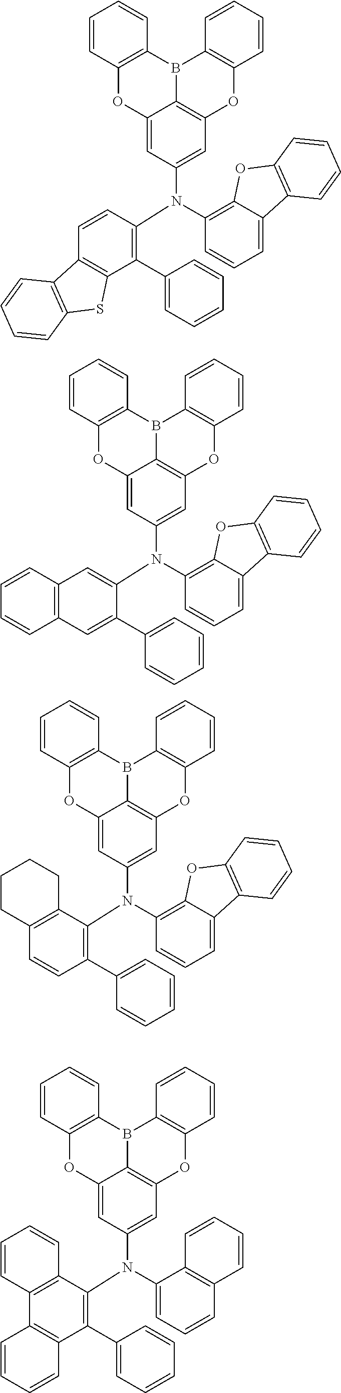

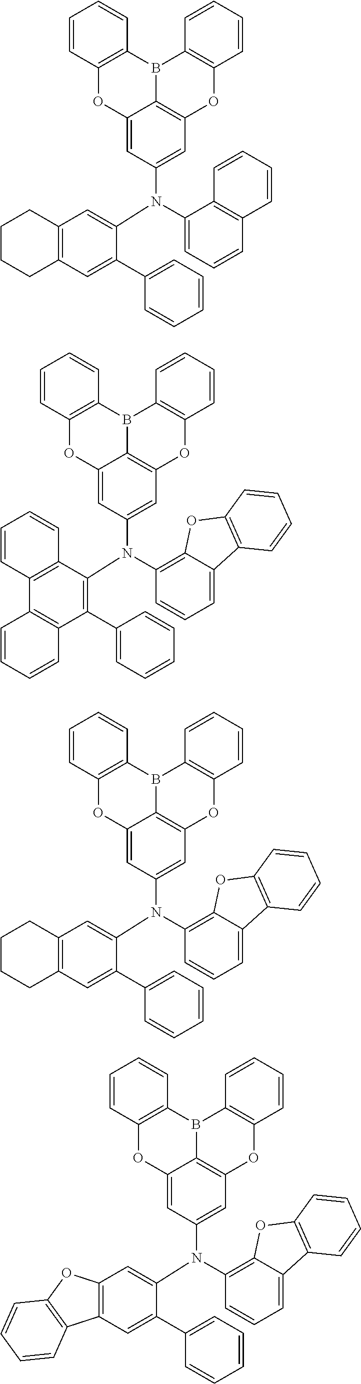

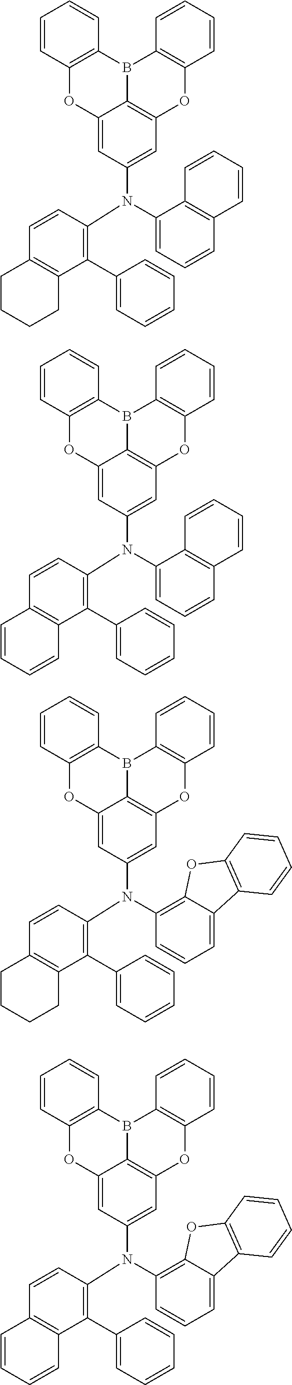

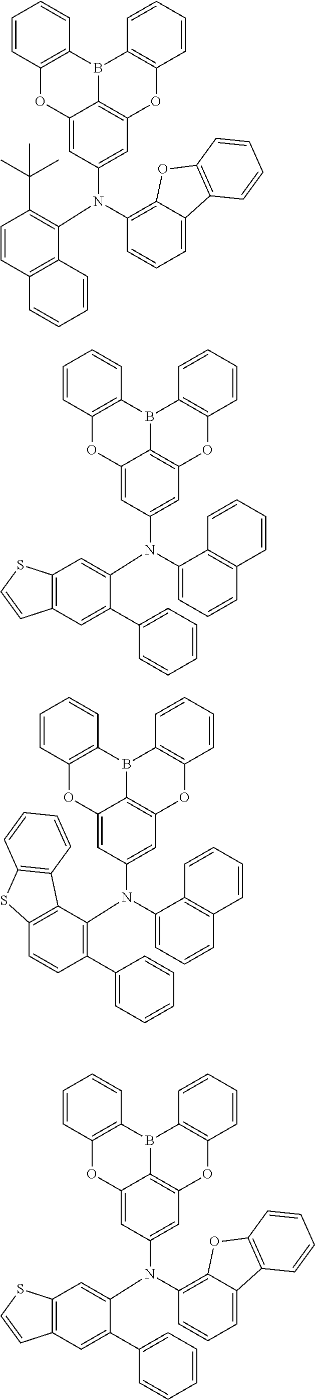

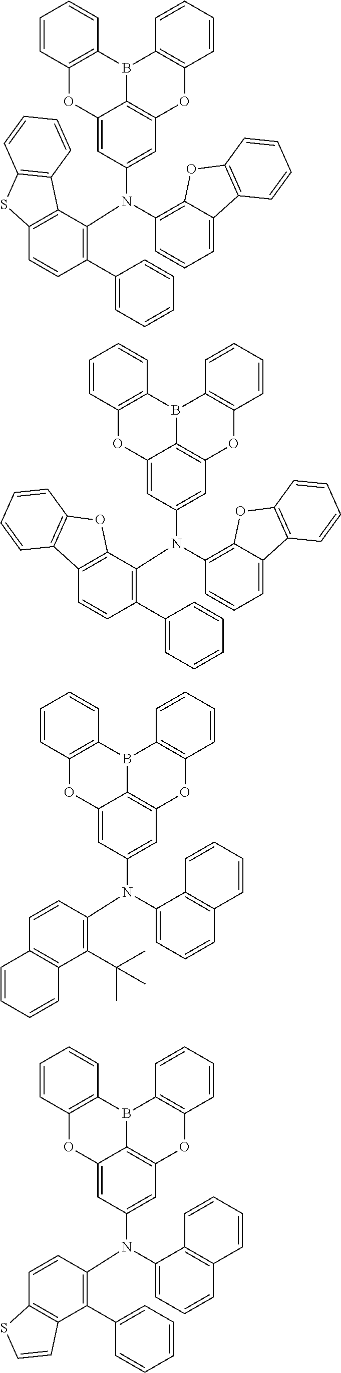

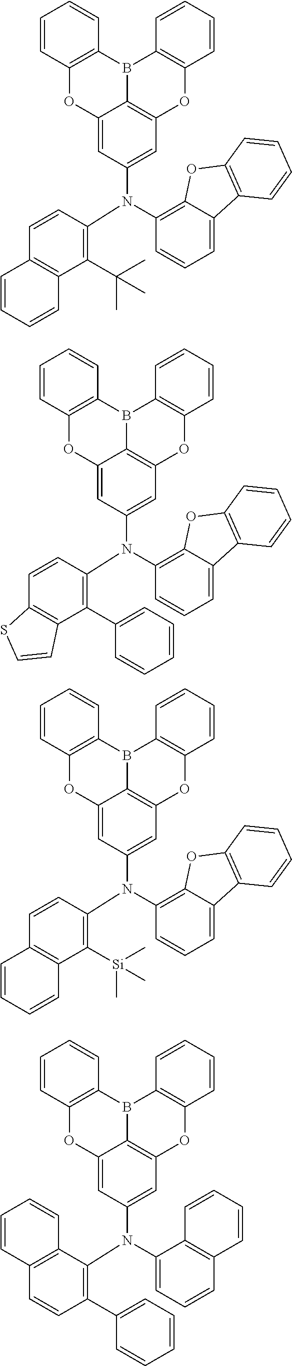

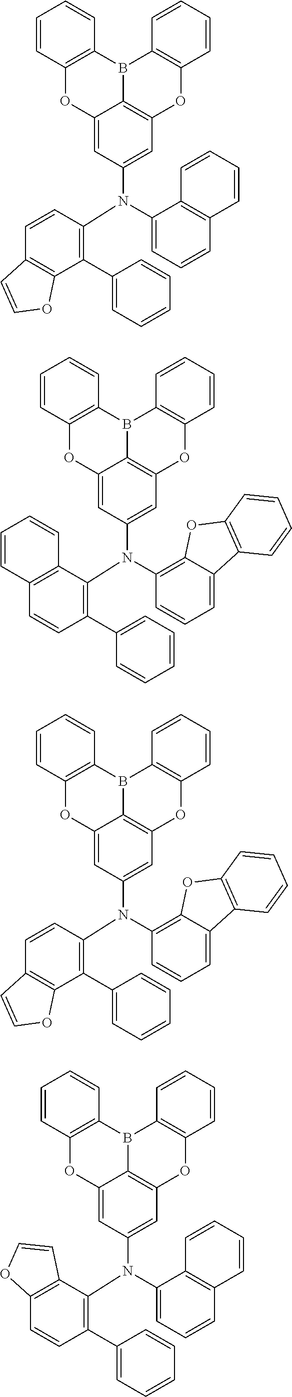 [SMILES: c1ccc(-c2c(N(c3cc4c5c(c3)Oc3ccccc3B5c3ccccc3O4)c3cccc4ccccc34)ccc3ccoc23)cc1 ; c1ccc(-c2ccc3ccccc3c2N(c2cc3c4c(c2)Oc2ccccc2B4c2ccccc2O3)c2cccc3c2oc2ccccc23)cc1 ; c1ccc(-c2c(N(c3cc4c5c(c3)Oc3ccccc3B5c3ccccc3O4)c3cccc4c3oc3ccccc34)ccc3ccoc23)cc1 ; c1ccc(-c2ccc3occc3c2N(c2cc3c4c(c2)Oc2ccccc2B4c2ccccc2O3)c2cccc3ccccc23)cc1]

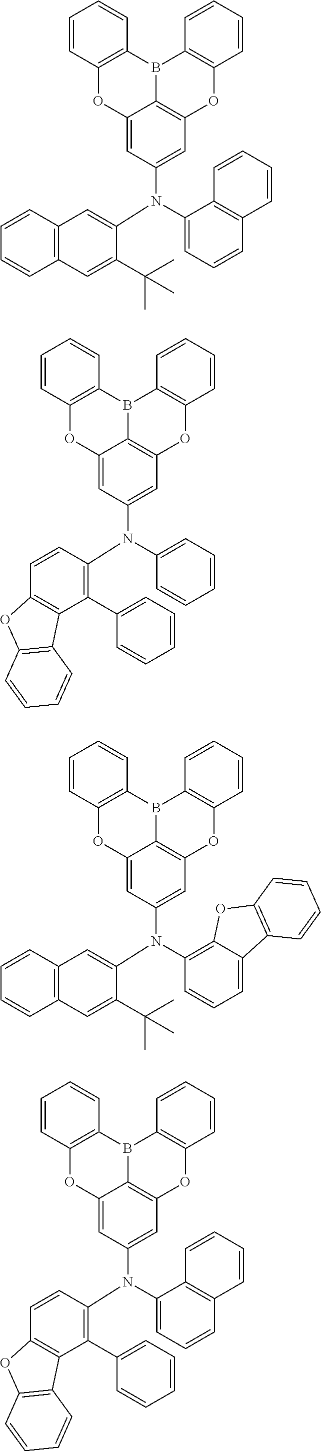 [SMILES: CC(C)(C)c1cc2ccccc2cc1N(c1cc2c3c(c1)Oc1ccccc1B3c1ccccc1O2)c1cccc2ccccc12 ; c1ccc(-c2c(N(c3ccccc3)c3cc4c5c(c3)Oc3ccccc3B5c3ccccc3O4)ccc3oc4ccccc4c23)cc1 ; CC(C)(C)c1cc2ccccc2cc1N(c1cc2c3c(c1)Oc1ccccc1B3c1ccccc1O2)c1cccc2c1oc1ccccc12 ; c1ccc(-c2c(N(c3cc4c5c(c3)Oc3ccccc3B5c3ccccc3O4)c3cccc4ccccc34)ccc3oc4ccccc4c23)cc1]

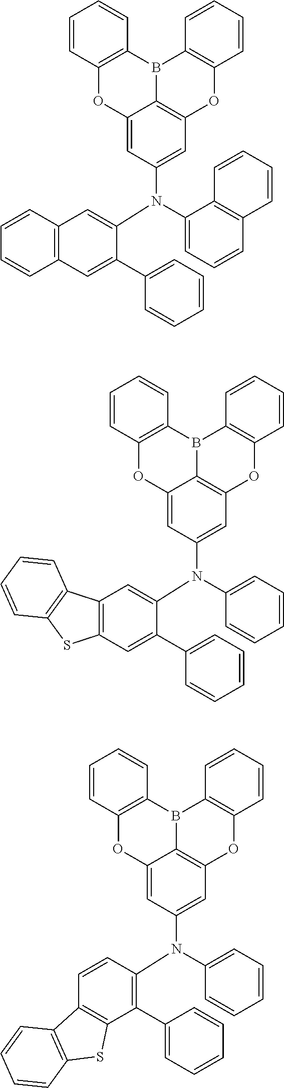 [SMILES: c1ccc(-c2cc3ccccc3cc2N(c2cc3c4c(c2)Oc2ccccc2B4c2ccccc2O3)c2cccc3ccccc23)cc1 ; c1ccc(-c2cc3sc4ccccc4c3cc2N(c2ccccc2)c2cc3c4c(c2)Oc2ccccc2B4c2ccccc2O3)cc1 ; c1ccc(-c2c(N(c3ccccc3)c3cc4c5c(c3)Oc3ccccc3B5c3ccccc3O4)ccc3c2sc2ccccc23)cc1]

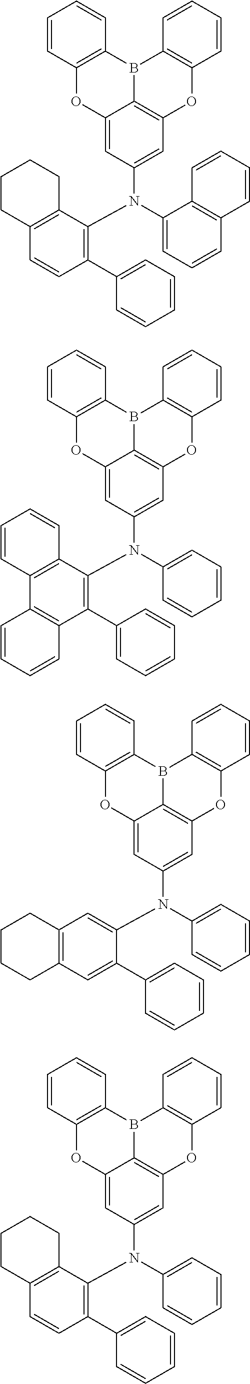

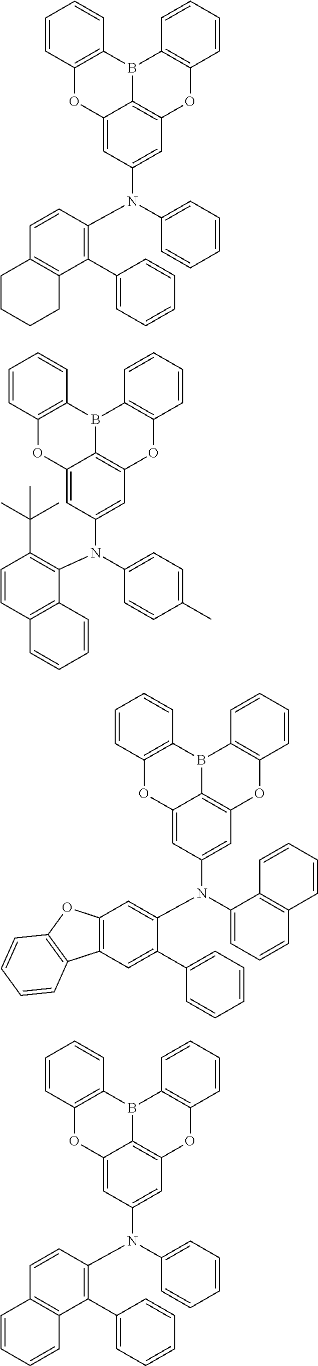

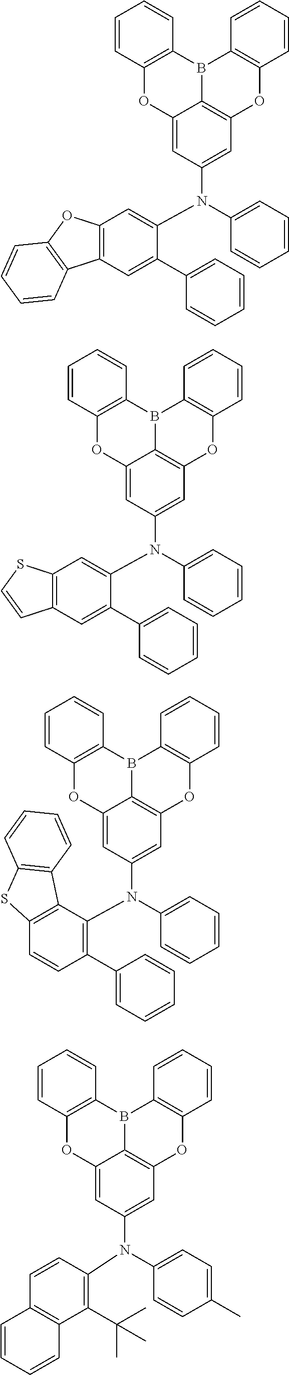

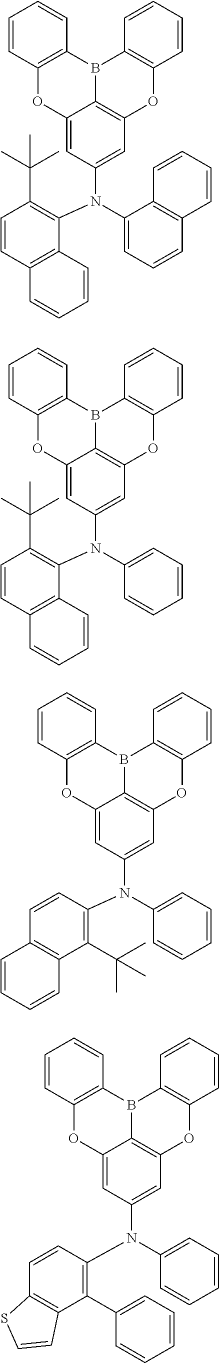

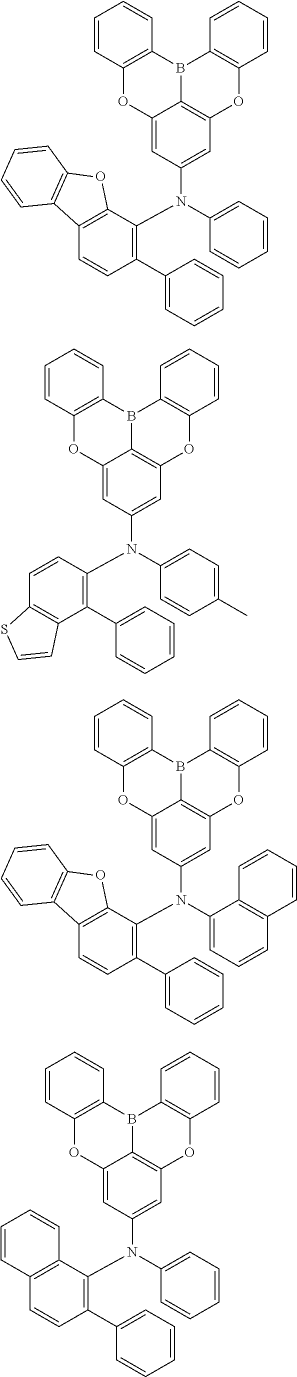

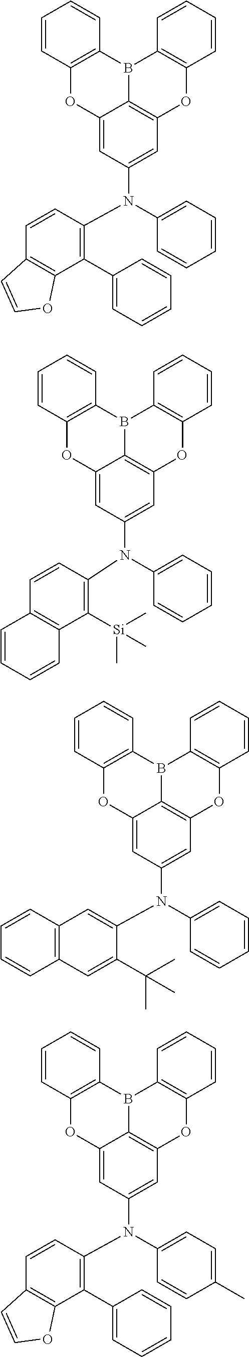

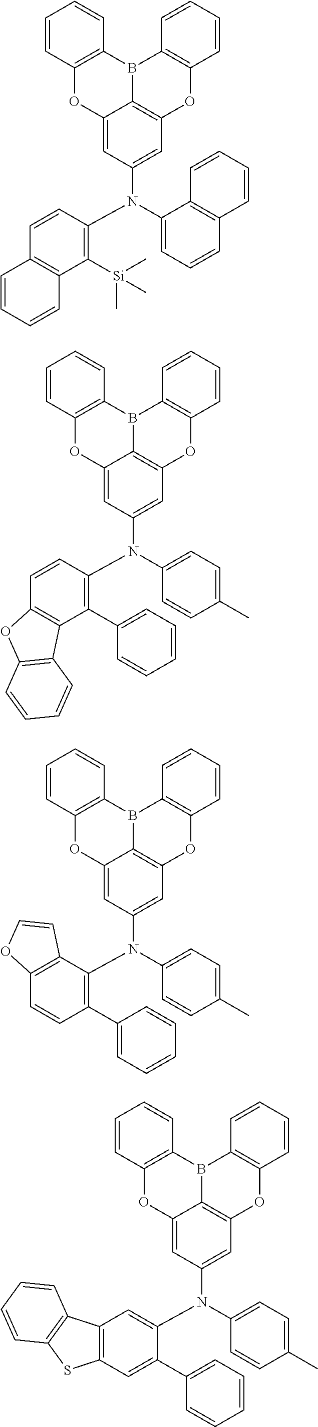

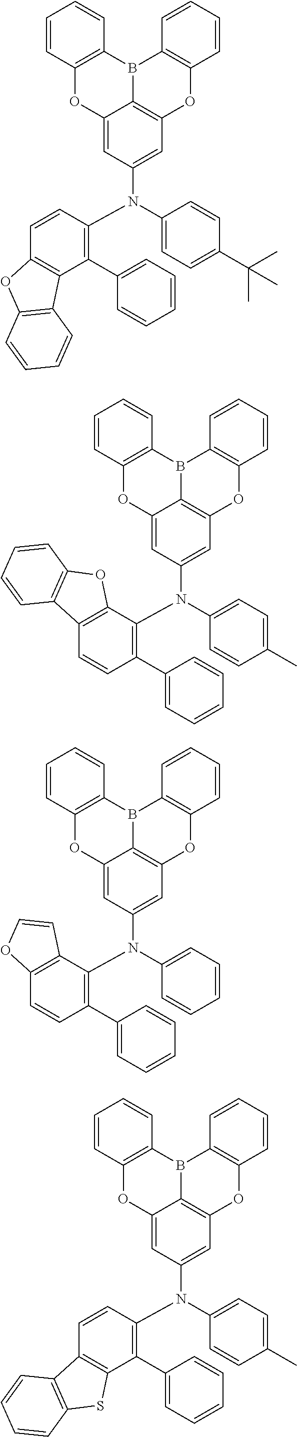

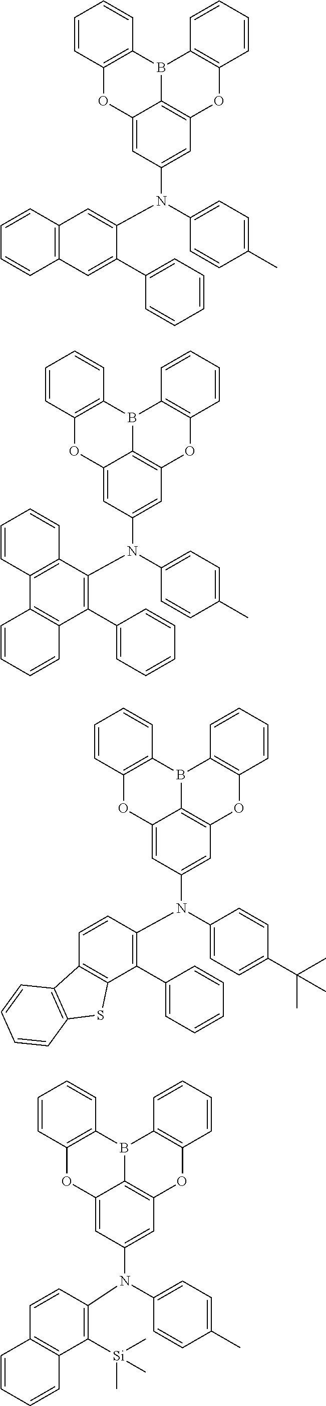

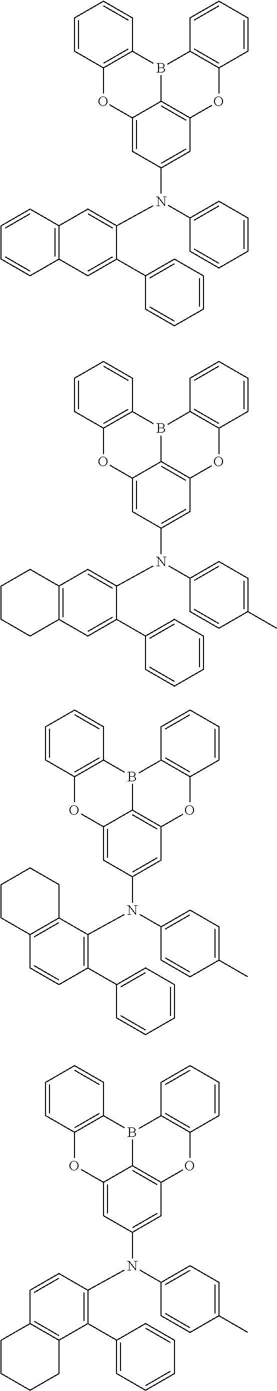

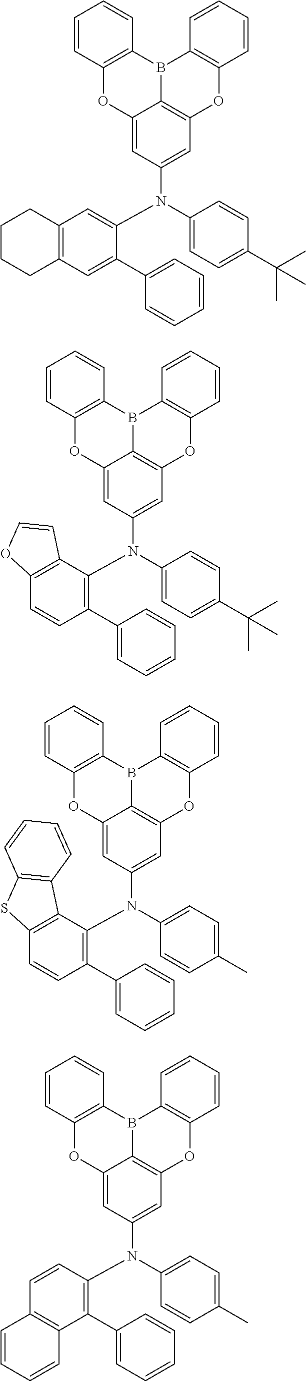

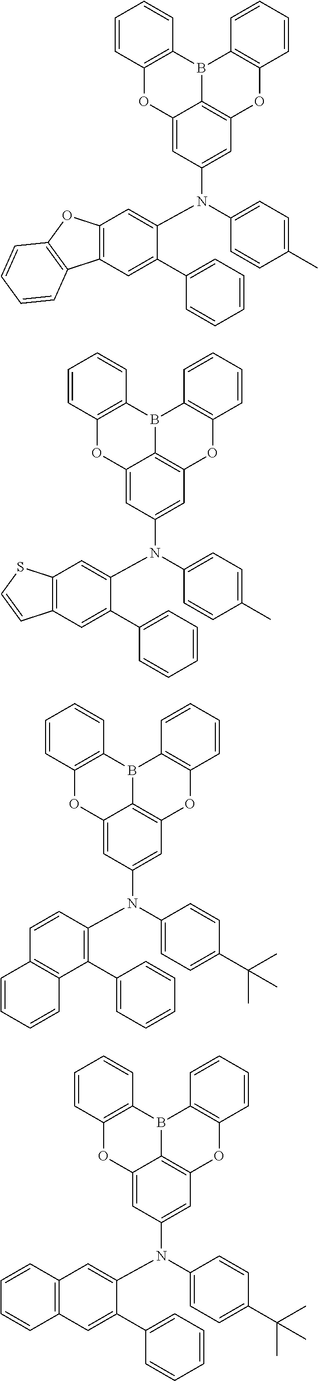

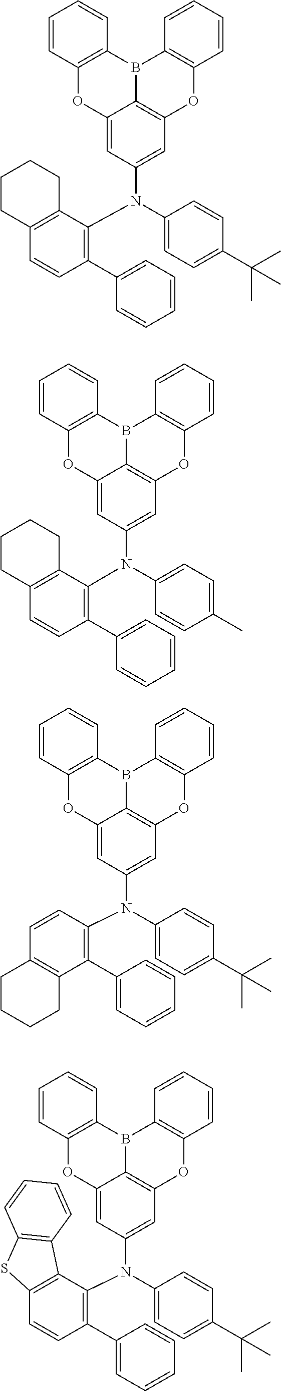

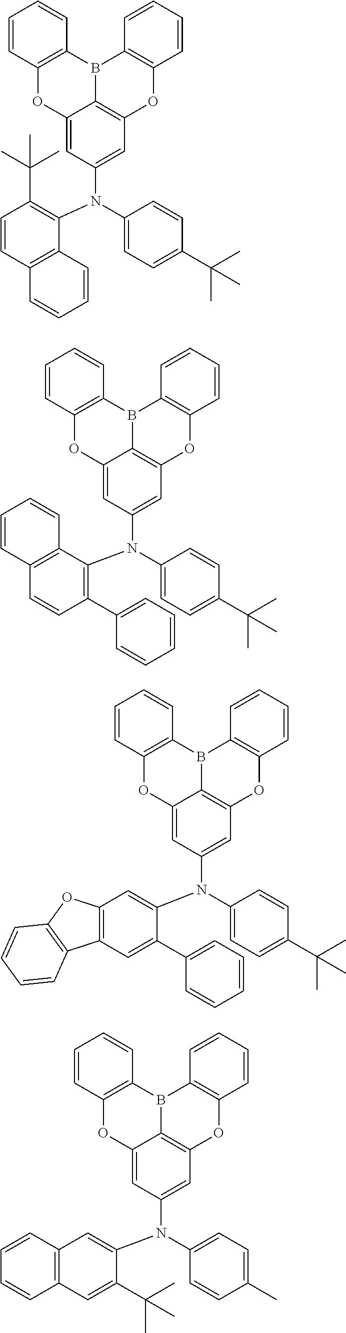

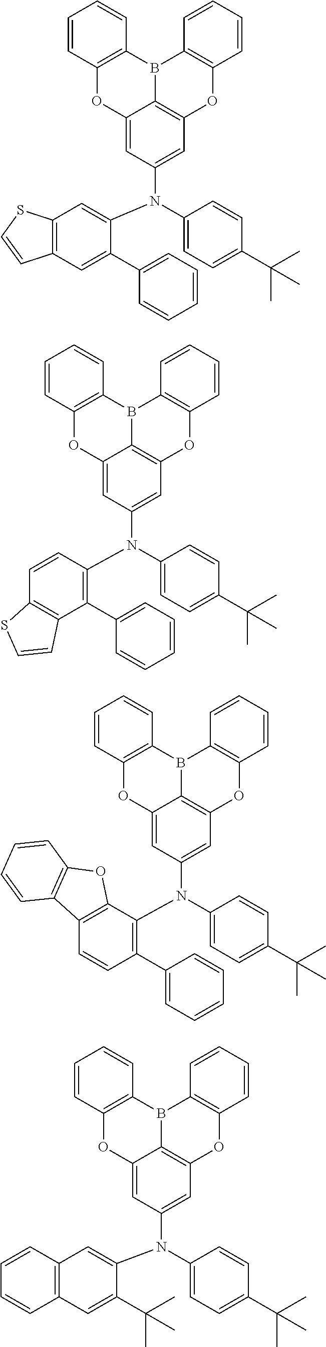

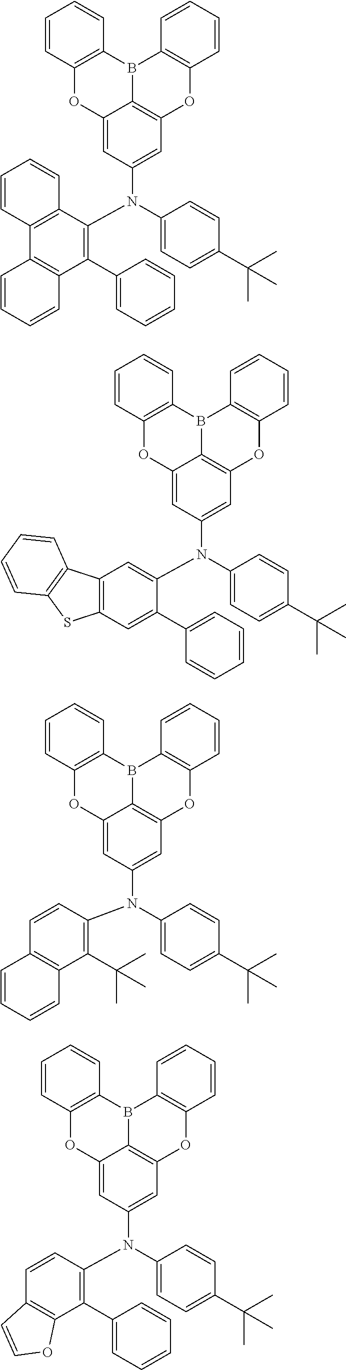

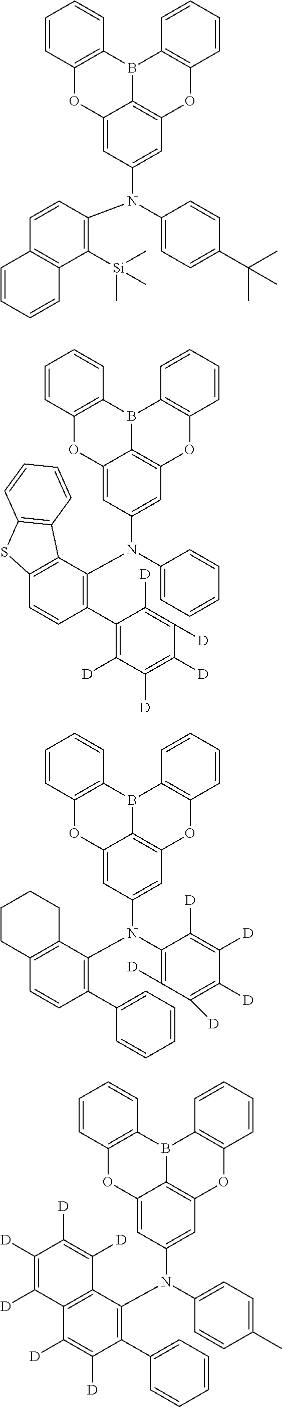

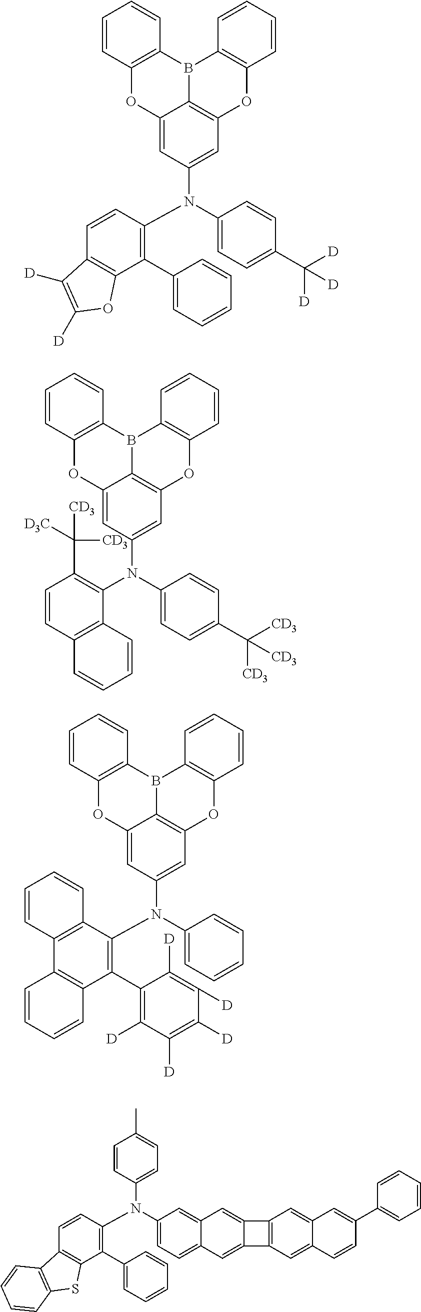

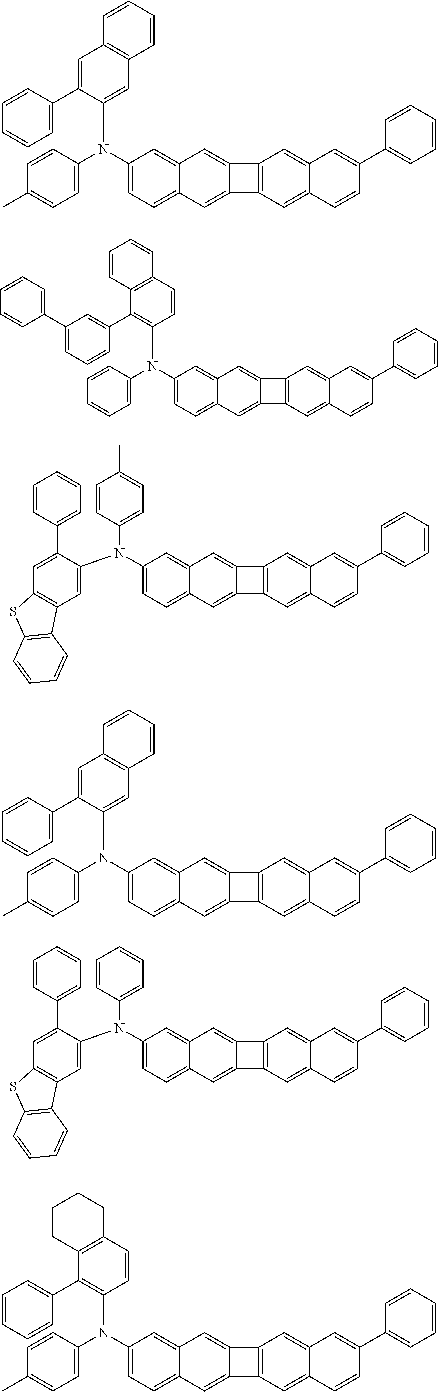

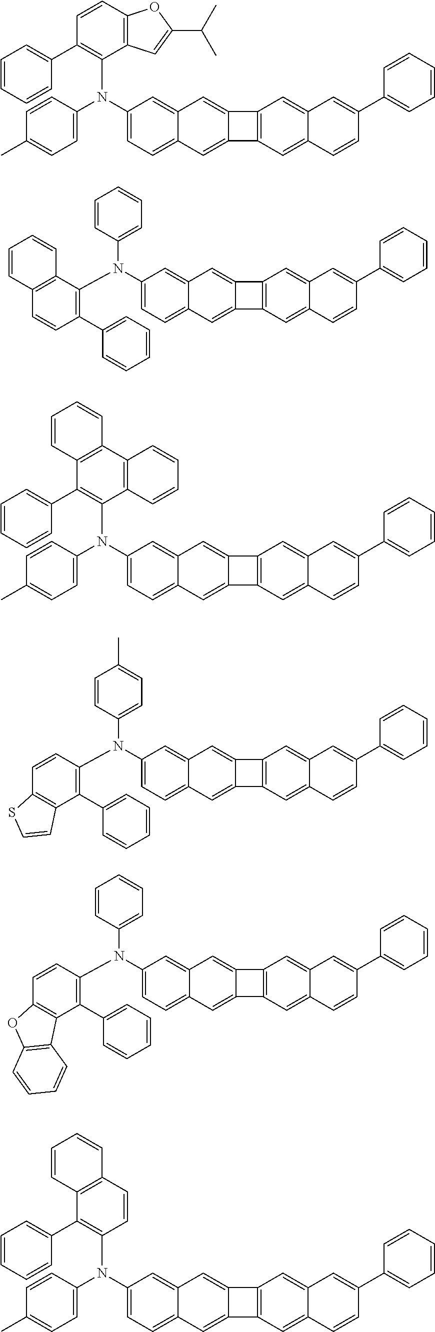

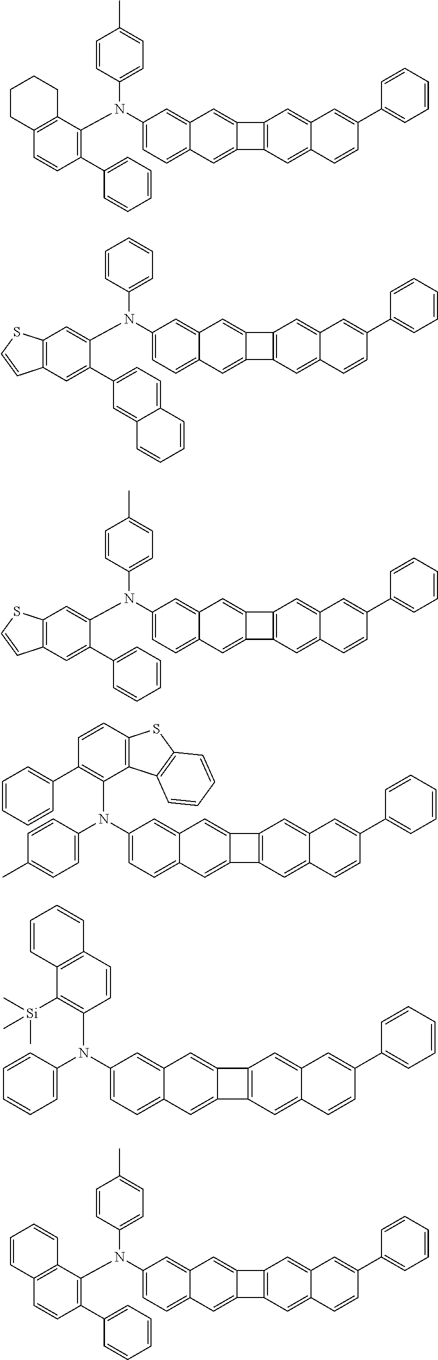

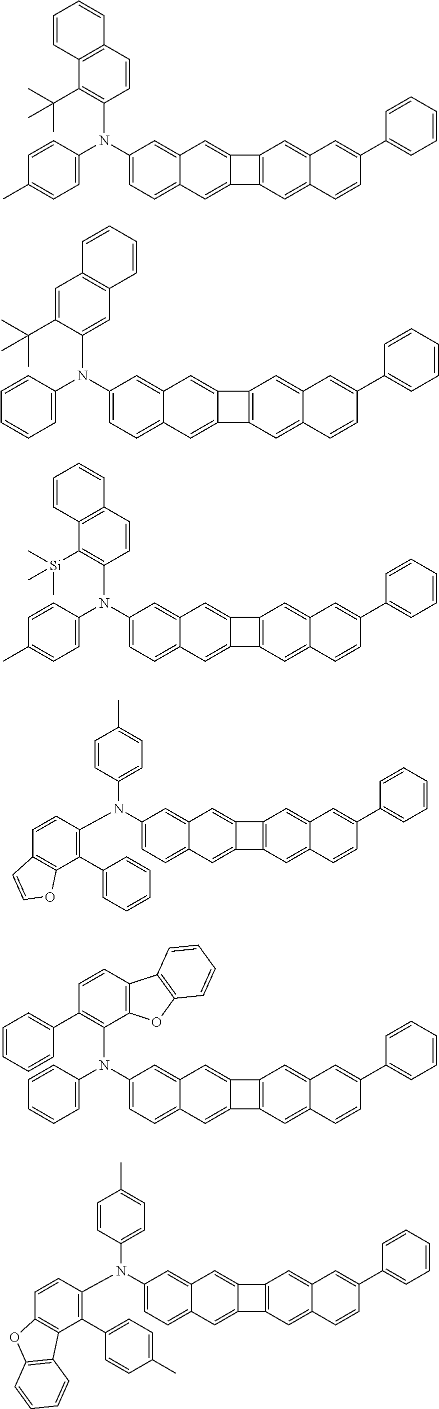

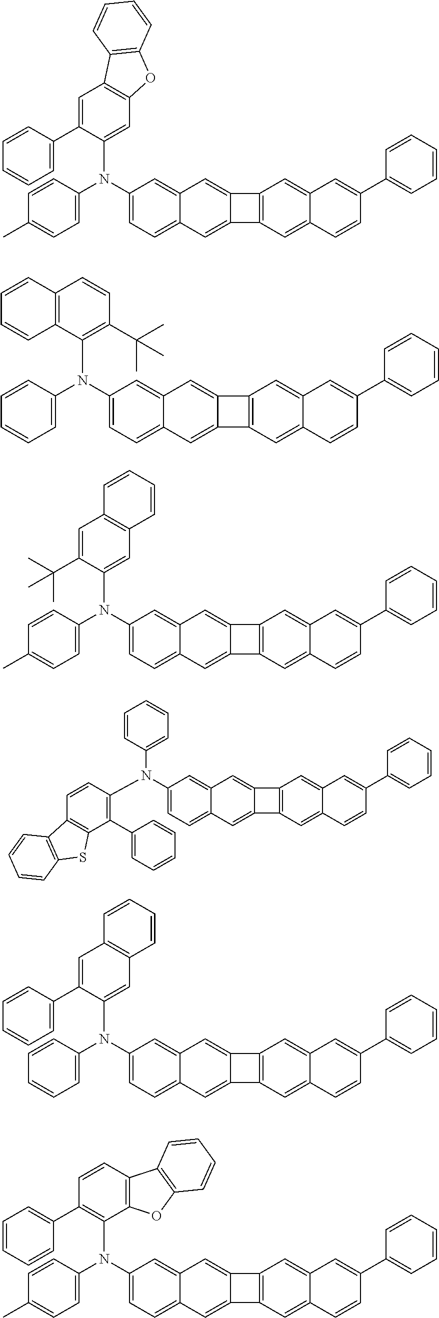

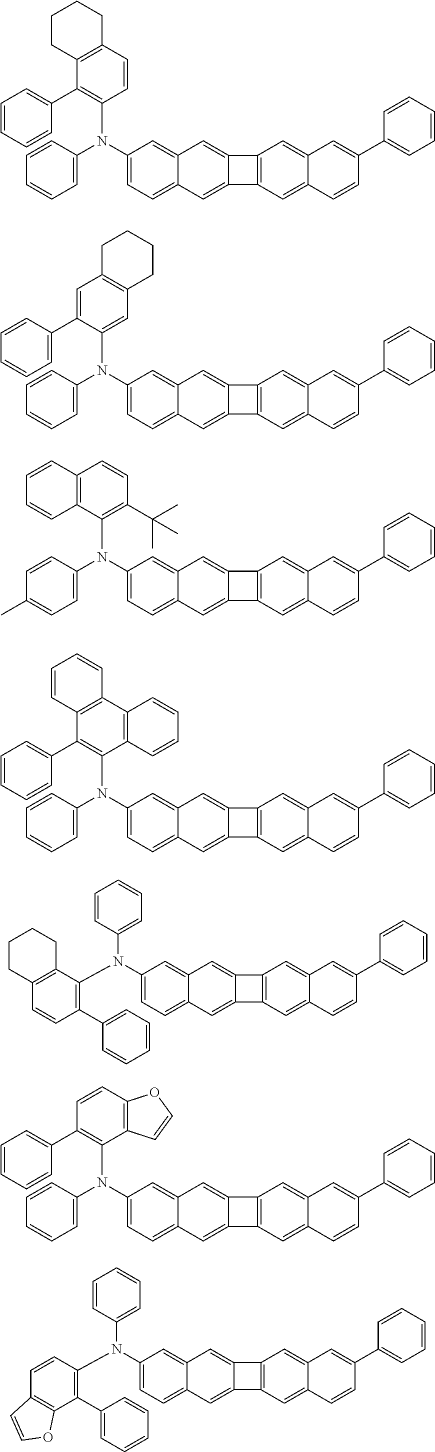

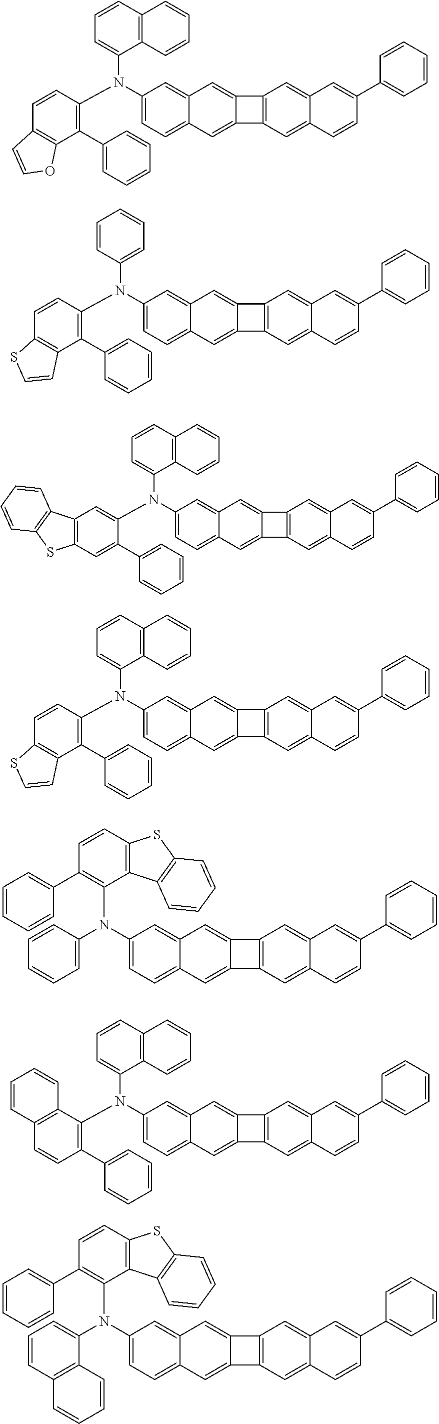

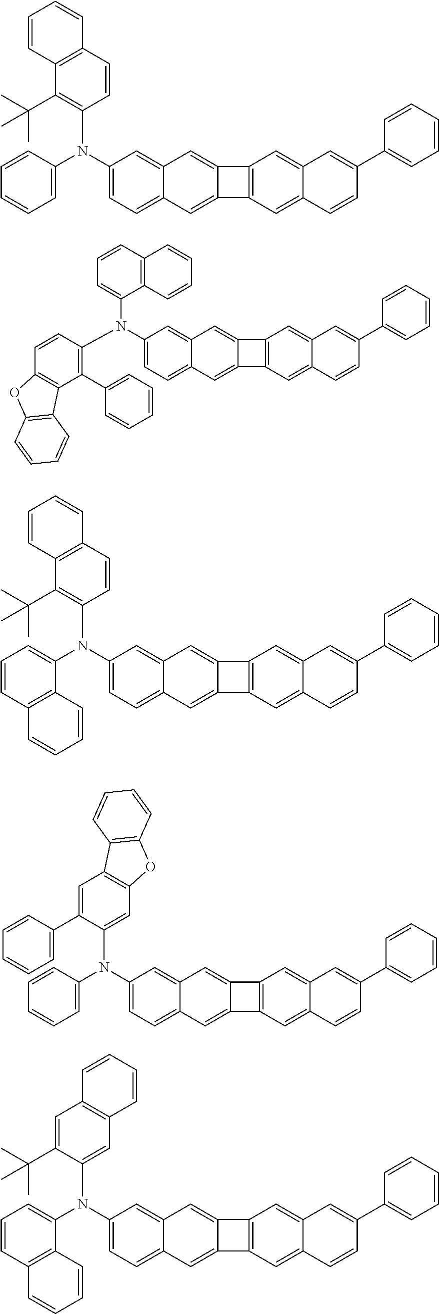

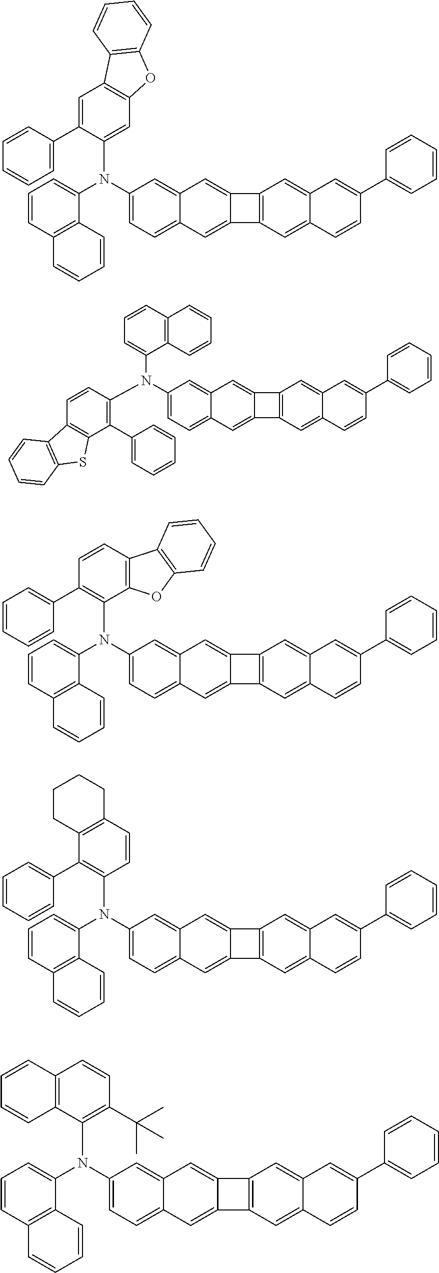

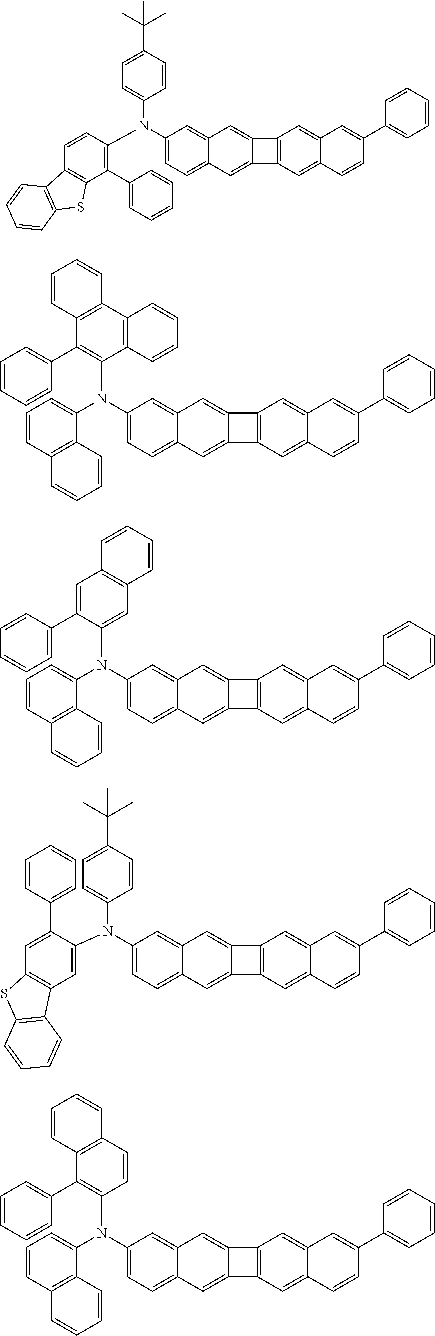

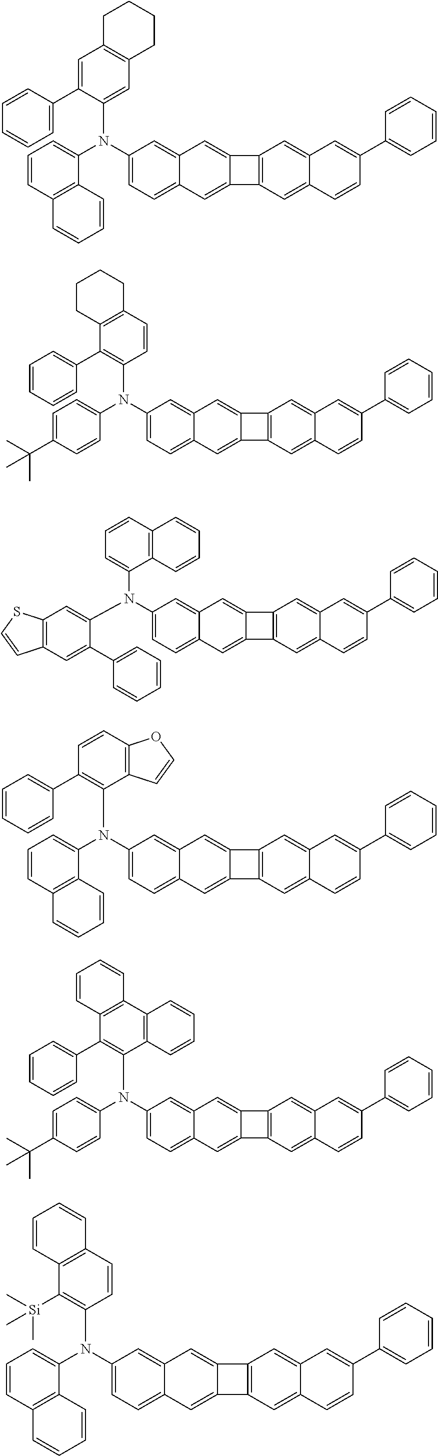

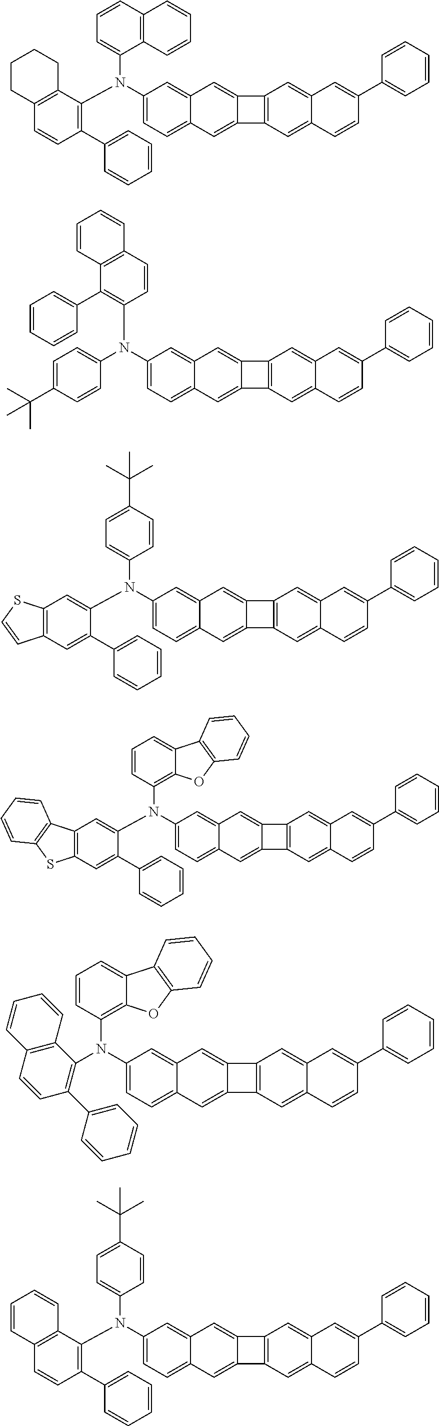

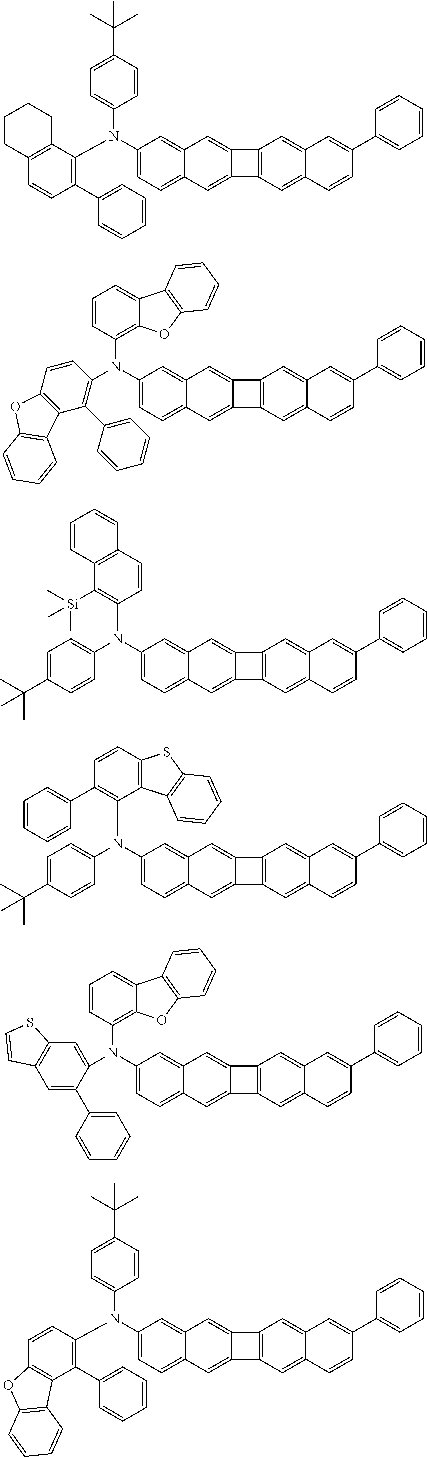

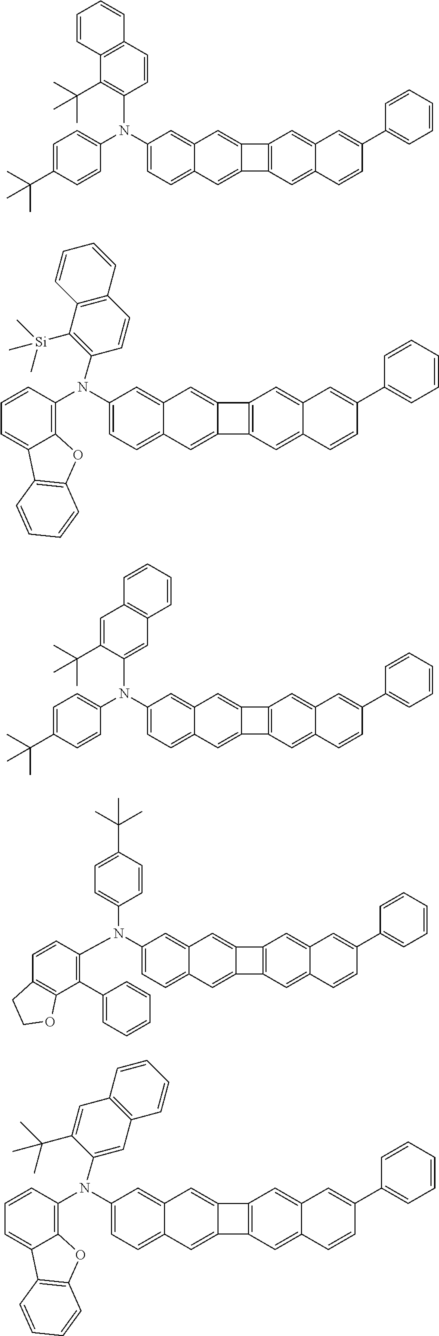

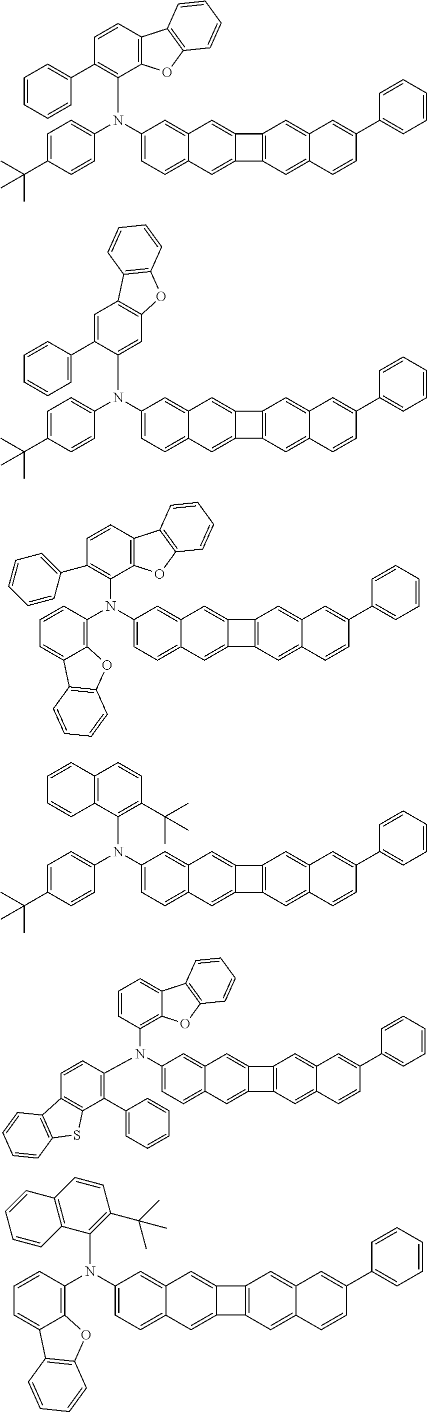

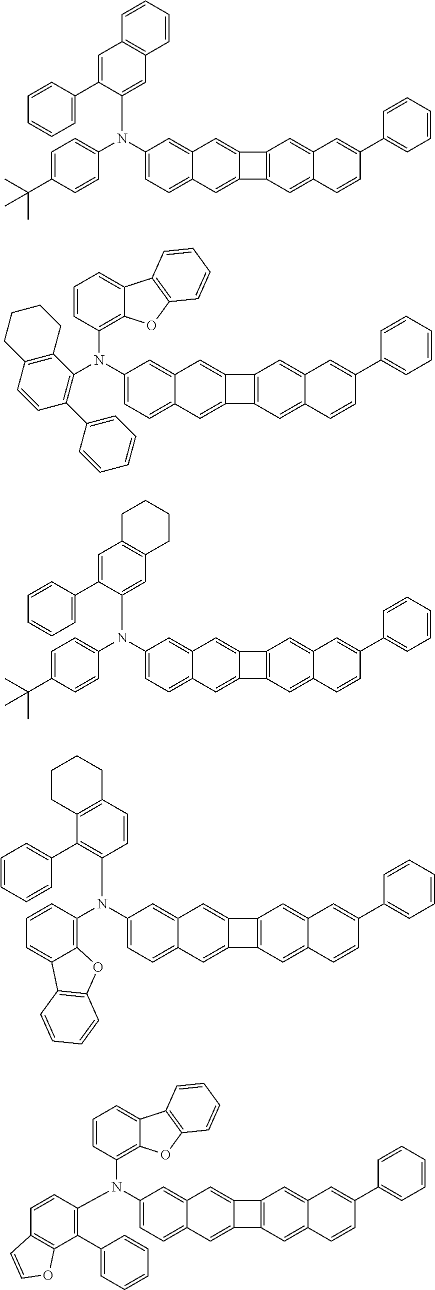 [SMILES: CC(C)(C)c1ccc(N(c2ccc3cc4c(cc3c2)-c2cc3cc(-c5ccccc5)ccc3cc2-4)c2cc3ccccc3cc2-c2ccccc2)cc1 ; c1ccc(-c2ccc3cc4c(cc3c2)-c2cc3cc(N(c5c(-c6ccccc6)ccc6c5CCCC6)c5cccc6c5oc5ccccc56)ccc3cc2-4)cc1 ; CC(C)(C)c1ccc(N(c2ccc3cc4c(cc3c2)-c2cc3cc(-c5ccccc5)ccc3cc2-4)c2cc3c(cc2-c2ccccc2)CCCC3)cc1 ; c1ccc(-c2ccc3cc4c(cc3c2)-c2cc3cc(N(c5ccc6c(c5-c5ccccc5)CCCC6)c5cccc6c5oc5ccccc56)ccc3cc2-4)cc1 ; c1ccc(-c2ccc3cc4c(cc3c2)-c2cc3cc(N(c5ccc6ccoc6c5-c5ccccc5)c5cccc6c5oc5ccccc56)ccc3cc2-4)cc1]

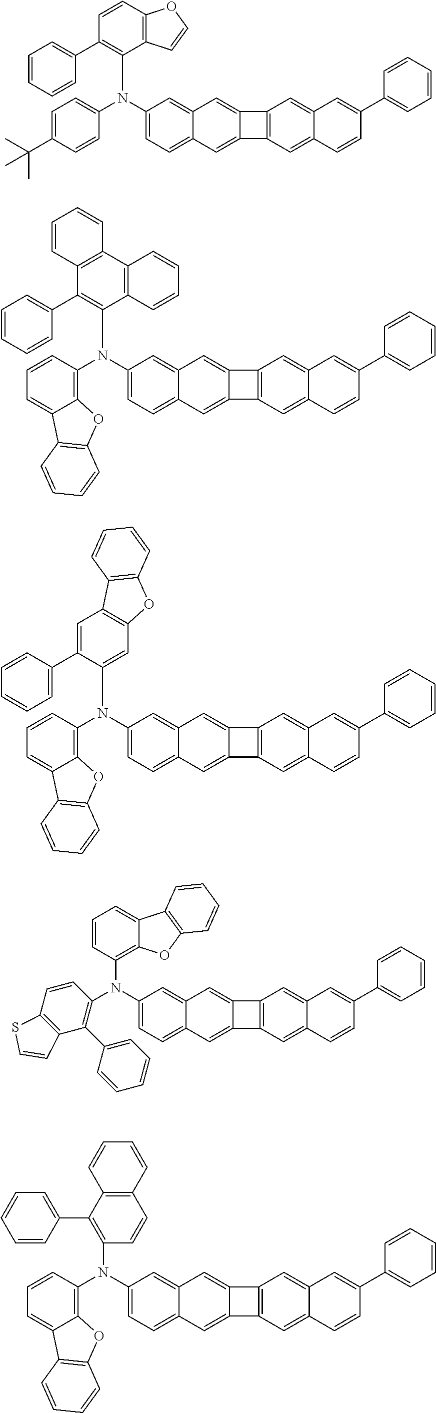

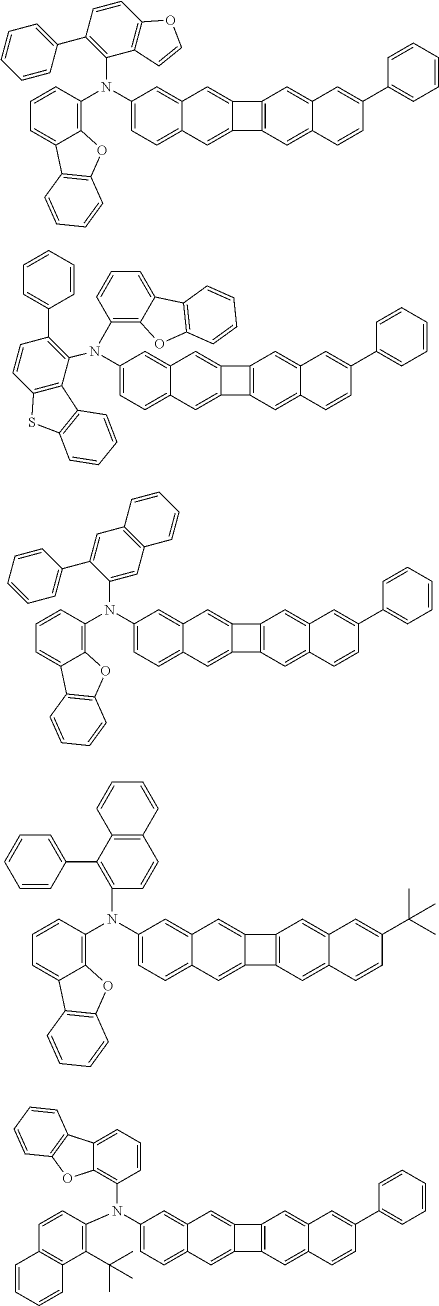

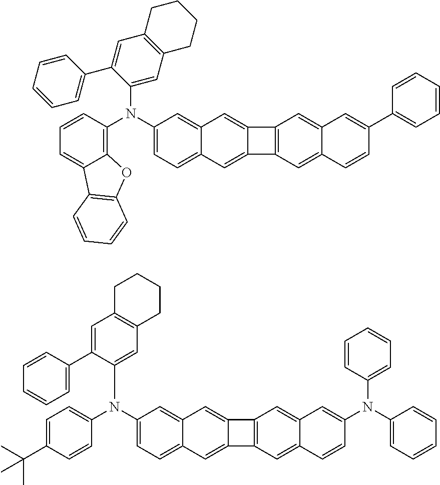 [SMILES: c1ccc(-c2ccc3cc4c(cc3c2)-c2cc3cc(N(c5cc6c(cc5-c5ccccc5)CCCC6)c5cccc6c5oc5ccccc56)ccc3cc2-4)cc1 ; CC(C)(C)c1ccc(N(c2ccc3cc4c(cc3c2)-c2cc3cc(N(c5ccccc5)c5ccccc5)ccc3cc2-4)c2cc3c(cc2-c2ccccc2)CCCC3)cc1]

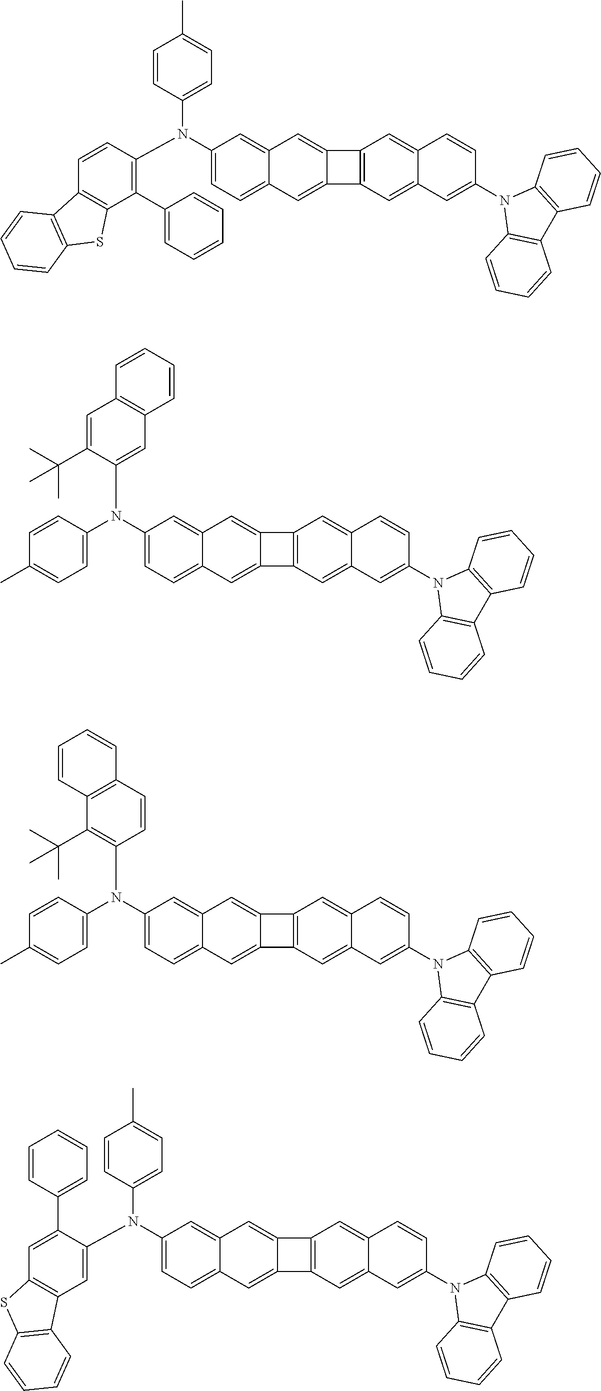 [SMILES: Cc1ccc(N(c2ccc3cc4c(cc3c2)-c2cc3ccc(-n5c6ccccc6c6ccccc65)cc3cc2-4)c2ccc3c(sc4ccccc43)c2-c2ccccc2)cc1 ; Cc1ccc(N(c2ccc3cc4c(cc3c2)-c2cc3ccc(-n5c6ccccc6c6ccccc65)cc3cc2-4)c2cc3ccccc3cc2C(C)(C)C)cc1 ; Cc1ccc(N(c2ccc3cc4c(cc3c2)-c2cc3ccc(-n5c6ccccc6c6ccccc65)cc3cc2-4)c2ccc3ccccc3c2C(C)(C)C)cc1 ; Cc1ccc(N(c2ccc3cc4c(cc3c2)-c2cc3ccc(-n5c6ccccc6c6ccccc65)cc3cc2-4)c2cc3c(cc2-c2ccccc2)sc2ccccc23)cc1]

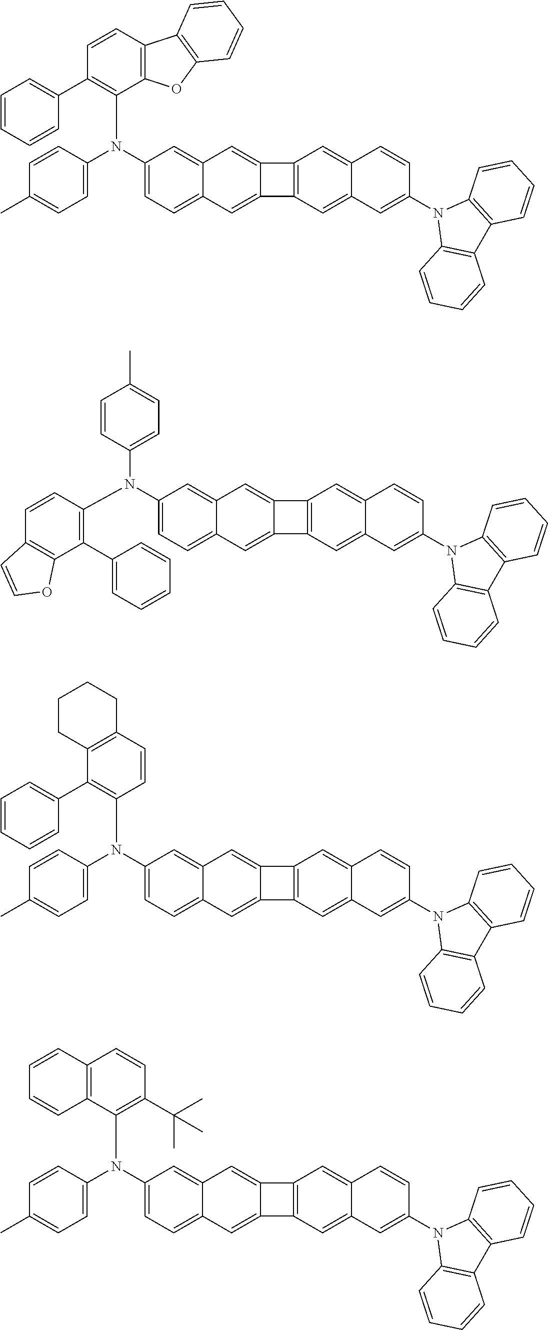 [SMILES: Cc1ccc(N(c2ccc3cc4c(cc3c2)-c2cc3ccc(-n5c6ccccc6c6ccccc65)cc3cc2-4)c2c(-c3ccccc3)ccc3c2oc2ccccc23)cc1 ; Cc1ccc(N(c2ccc3cc4c(cc3c2)-c2cc3ccc(-n5c6ccccc6c6ccccc65)cc3cc2-4)c2ccc3ccoc3c2-c2ccccc2)cc1 ; Cc1ccc(N(c2ccc3cc4c(cc3c2)-c2cc3ccc(-n5c6ccccc6c6ccccc65)cc3cc2-4)c2ccc3c(c2-c2ccccc2)CCCC3)cc1 ; Cc1ccc(N(c2ccc3cc4c(cc3c2)-c2cc3ccc(-n5c6ccccc6c6ccccc65)cc3cc2-4)c2c(C(C)(C)C)ccc3ccccc23)cc1]

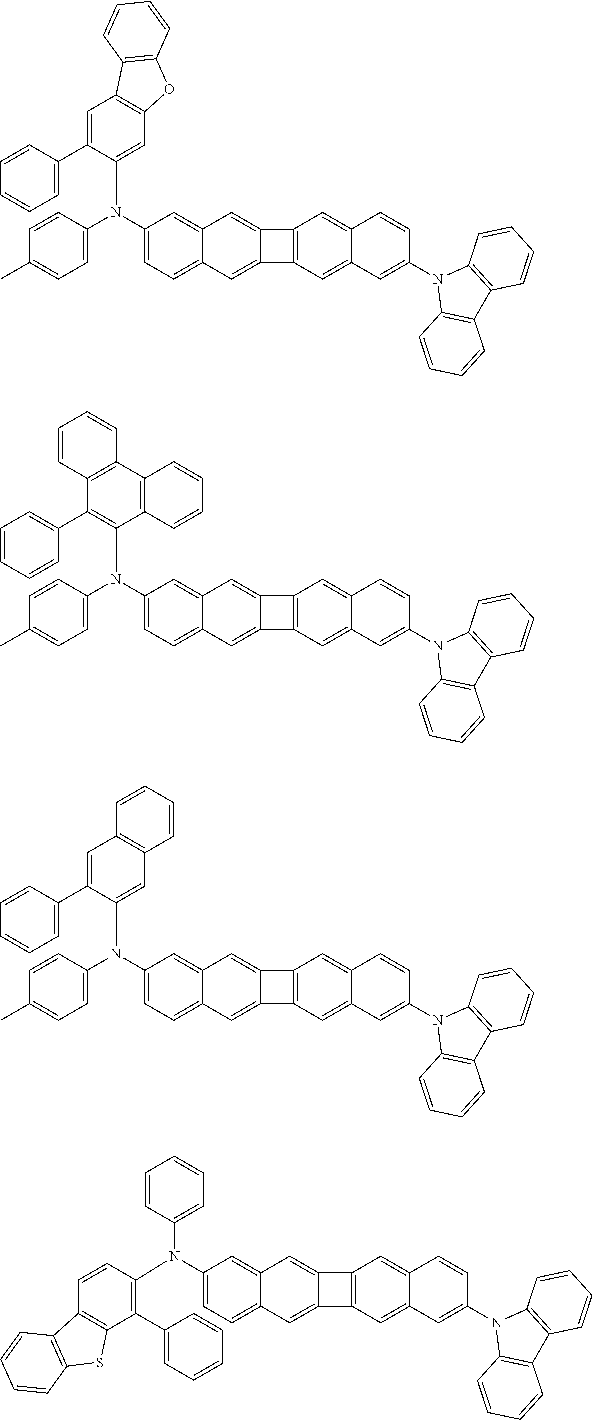 [SMILES: Cc1ccc(N(c2ccc3cc4c(cc3c2)-c2cc3ccc(-n5c6ccccc6c6ccccc65)cc3cc2-4)c2cc3oc4ccccc4c3cc2-c2ccccc2)cc1 ; Cc1ccc(N(c2ccc3cc4c(cc3c2)-c2cc3ccc(-n5c6ccccc6c6ccccc65)cc3cc2-4)c2c(-c3ccccc3)c3ccccc3c3ccccc23)cc1 ; Cc1ccc(N(c2ccc3cc4c(cc3c2)-c2cc3ccc(-n5c6ccccc6c6ccccc65)cc3cc2-4)c2cc3ccccc3cc2-c2ccccc2)cc1 ; c1ccc(-c2c(N(c3ccccc3)c3ccc4cc5c(cc4c3)-c3cc4ccc(-n6c7ccccc7c7ccccc76)cc4cc3-5)ccc3c2sc2ccccc23)cc1]

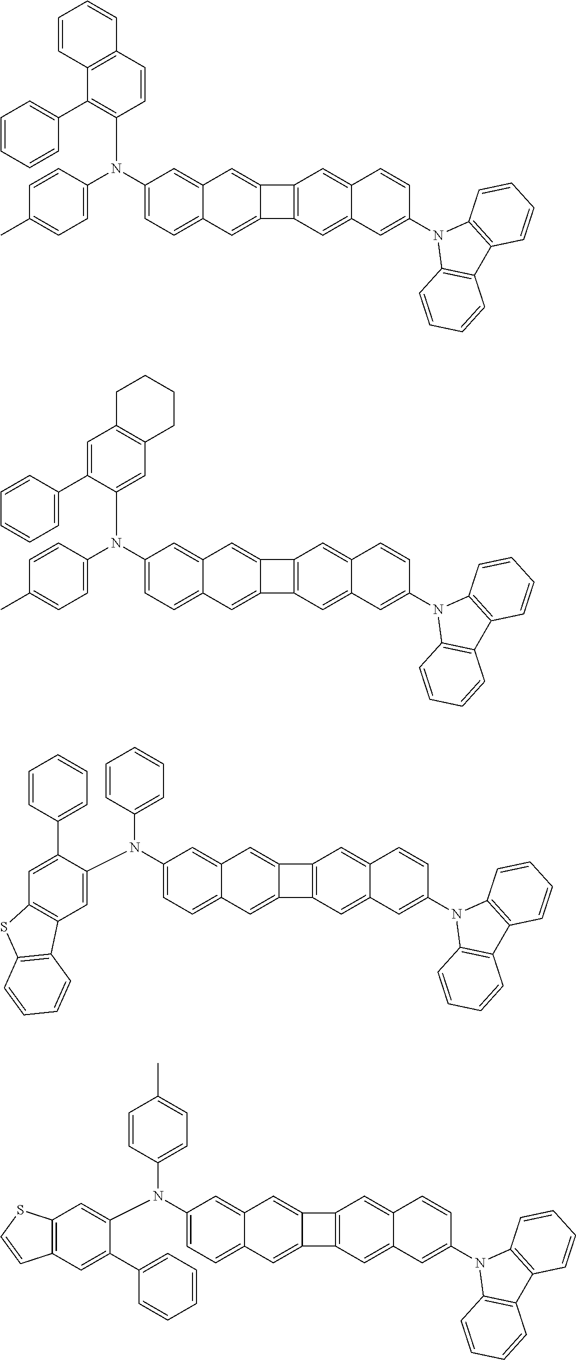 [SMILES: Cc1ccc(N(c2ccc3cc4c(cc3c2)-c2cc3ccc(-n5c6ccccc6c6ccccc65)cc3cc2-4)c2ccc3ccccc3c2-c2ccccc2)cc1 ; Cc1ccc(N(c2ccc3cc4c(cc3c2)-c2cc3ccc(-n5c6ccccc6c6ccccc65)cc3cc2-4)c2cc3c(cc2-c2ccccc2)CCCC3)cc1 ; c1ccc(-c2cc3sc4ccccc4c3cc2N(c2ccccc2)c2ccc3cc4c(cc3c2)-c2cc3ccc(-n5c6ccccc6c6ccccc65)cc3cc2-4)cc1 ; Cc1ccc(N(c2ccc3cc4c(cc3c2)-c2cc3ccc(-n5c6ccccc6c6ccccc65)cc3cc2-4)c2cc3sccc3cc2-c2ccccc2)cc1]

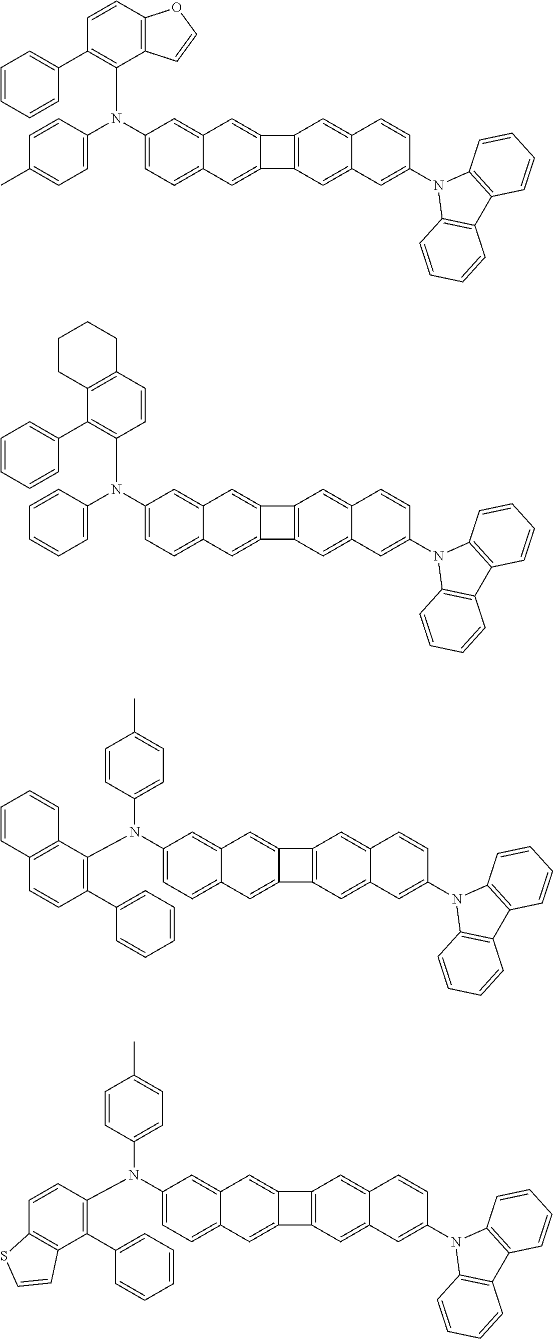 [SMILES: Cc1ccc(N(c2ccc3cc4c(cc3c2)-c2cc3ccc(-n5c6ccccc6c6ccccc65)cc3cc2-4)c2c(-c3ccccc3)ccc3occc23)cc1 ; c1ccc(-c2c(N(c3ccccc3)c3ccc4cc5c(cc4c3)-c3cc4ccc(-n6c7ccccc7c7ccccc76)cc4cc3-5)ccc3c2CCCC3)cc1 ; Cc1ccc(N(c2ccc3cc4c(cc3c2)-c2cc3ccc(-n5c6ccccc6c6ccccc65)cc3cc2-4)c2c(-c3ccccc3)ccc3ccccc23)cc1 ; Cc1ccc(N(c2ccc3cc4c(cc3c2)-c2cc3ccc(-n5c6ccccc6c6ccccc65)cc3cc2-4)c2ccc3sccc3c2-c2ccccc2)cc1]

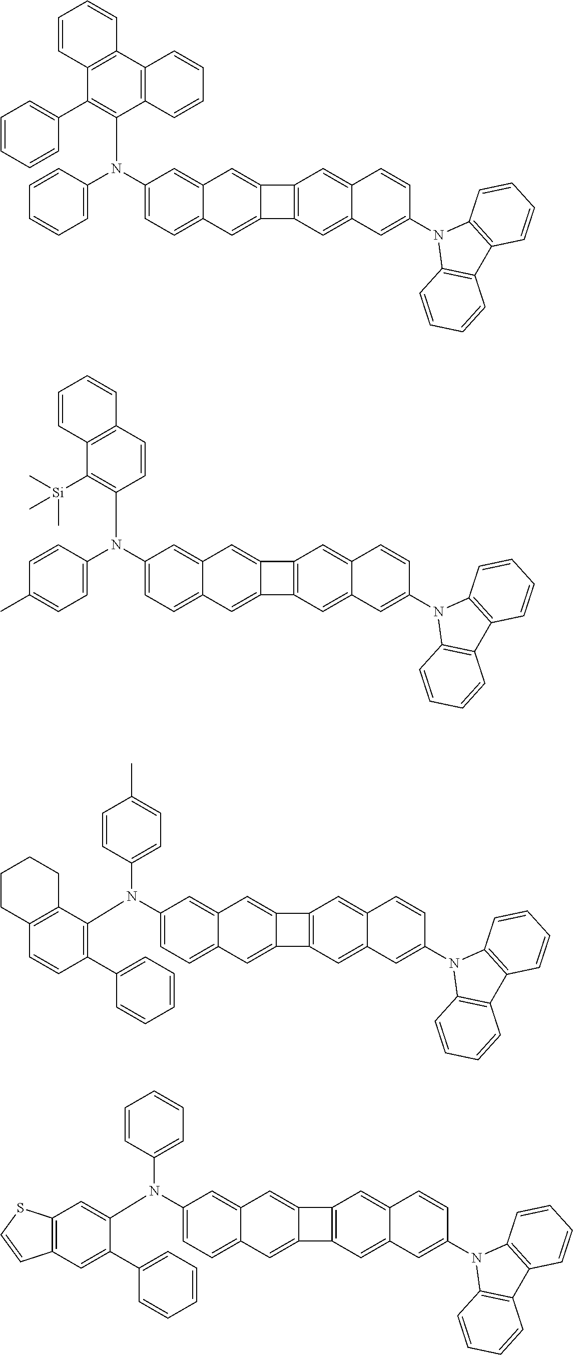 [SMILES: c1ccc(-c2c(N(c3ccccc3)c3ccc4cc5c(cc4c3)-c3cc4ccc(-n6c7ccccc7c7ccccc76)cc4cc3-5)c3ccccc3c3ccccc23)cc1 ; Cc1ccc(N(c2ccc3cc4c(cc3c2)-c2cc3ccc(-n5c6ccccc6c6ccccc65)cc3cc2-4)c2ccc3ccccc3c2[Si](C)(C)C)cc1 ; Cc1ccc(N(c2ccc3cc4c(cc3c2)-c2cc3ccc(-n5c6ccccc6c6ccccc65)cc3cc2-4)c2c(-c3ccccc3)ccc3c2CCCC3)cc1 ; c1ccc(-c2cc3ccsc3cc2N(c2ccccc2)c2ccc3cc4c(cc3c2)-c2cc3ccc(-n5c6ccccc6c6ccccc65)cc3cc2-4)cc1]

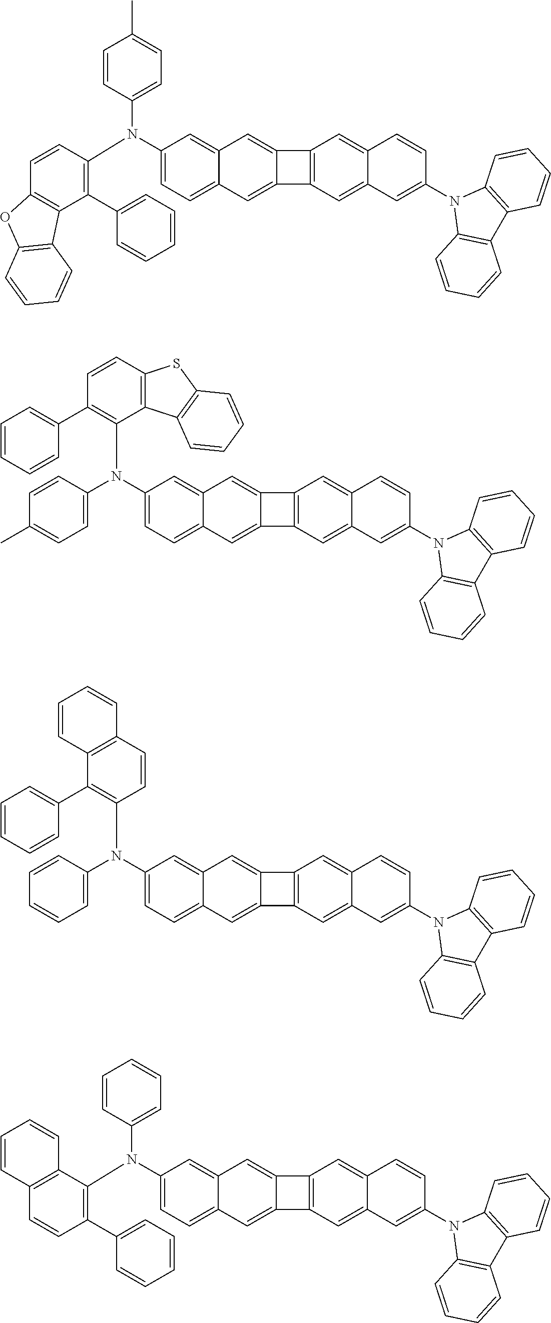 [SMILES: Cc1ccc(N(c2ccc3cc4c(cc3c2)-c2cc3ccc(-n5c6ccccc6c6ccccc65)cc3cc2-4)c2ccc3oc4ccccc4c3c2-c2ccccc2)cc1 ; Cc1ccc(N(c2ccc3cc4c(cc3c2)-c2cc3ccc(-n5c6ccccc6c6ccccc65)cc3cc2-4)c2c(-c3ccccc3)ccc3sc4ccccc4c23)cc1 ; c1ccc(-c2c(N(c3ccccc3)c3ccc4cc5c(cc4c3)-c3cc4ccc(-n6c7ccccc7c7ccccc76)cc4cc3-5)ccc3ccccc23)cc1 ; c1ccc(-c2ccc3ccccc3c2N(c2ccccc2)c2ccc3cc4c(cc3c2)-c2cc3ccc(-n5c6ccccc6c6ccccc65)cc3cc2-4)cc1]

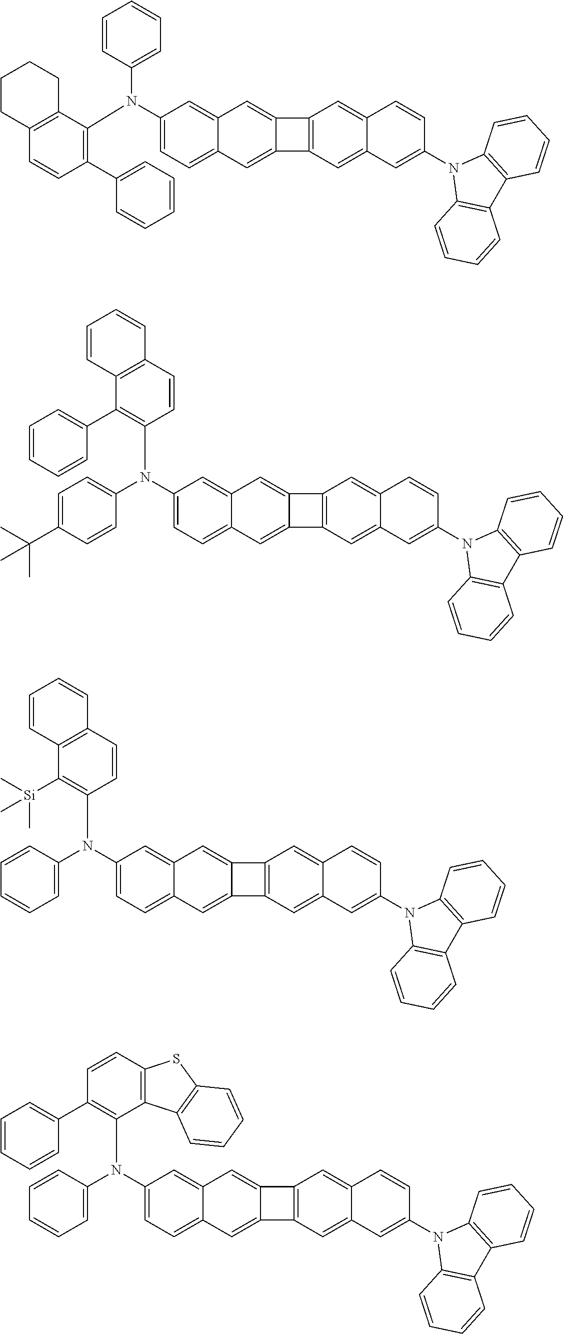 [SMILES: c1ccc(-c2ccc3c(c2N(c2ccccc2)c2ccc4cc5c(cc4c2)-c2cc4ccc(-n6c7ccccc7c7ccccc76)cc4cc2-5)CCCC3)cc1 ; CC(C)(C)c1ccc(N(c2ccc3cc4c(cc3c2)-c2cc3ccc(-n5c6ccccc6c6ccccc65)cc3cc2-4)c2ccc3ccccc3c2-c2ccccc2)cc1 ; C[Si](C)(C)c1c(N(c2ccccc2)c2ccc3cc4c(cc3c2)-c2cc3ccc(-n5c6ccccc6c6ccccc65)cc3cc2-4)ccc2ccccc12 ; c1ccc(-c2ccc3sc4ccccc4c3c2N(c2ccccc2)c2ccc3cc4c(cc3c2)-c2cc3ccc(-n5c6ccccc6c6ccccc65)cc3cc2-4)cc1]

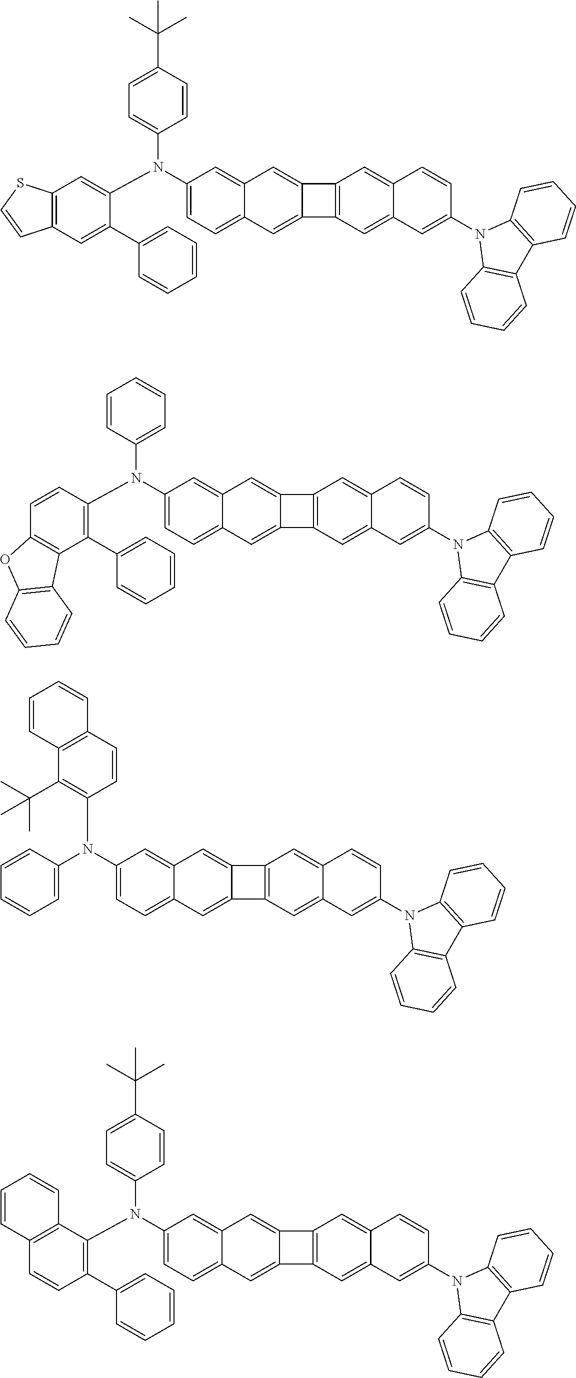

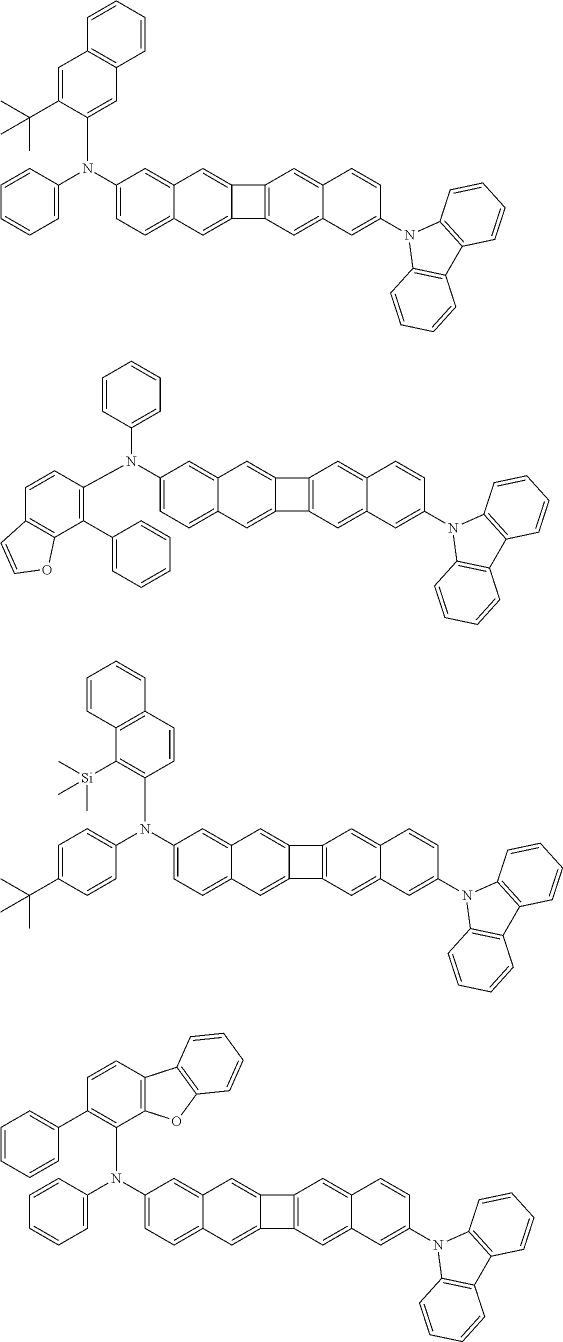 [SMILES: CC(C)(C)c1cc2ccccc2cc1N(c1ccccc1)c1ccc2cc3c(cc2c1)-c1cc2ccc(-n4c5ccccc5c5ccccc54)cc2cc1-3 ; c1ccc(-c2c(N(c3ccccc3)c3ccc4cc5c(cc4c3)-c3cc4ccc(-n6c7ccccc7c7ccccc76)cc4cc3-5)ccc3ccoc23)cc1 ; CC(C)(C)c1ccc(N(c2ccc3cc4c(cc3c2)-c2cc3ccc(-n5c6ccccc6c6ccccc65)cc3cc2-4)c2ccc3ccccc3c2[Si](C)(C)C)cc1 ; c1ccc(-c2ccc3c(oc4ccccc43)c2N(c2ccccc2)c2ccc3cc4c(cc3c2)-c2cc3ccc(-n5c6ccccc6c6ccccc65)cc3cc2-4)cc1]

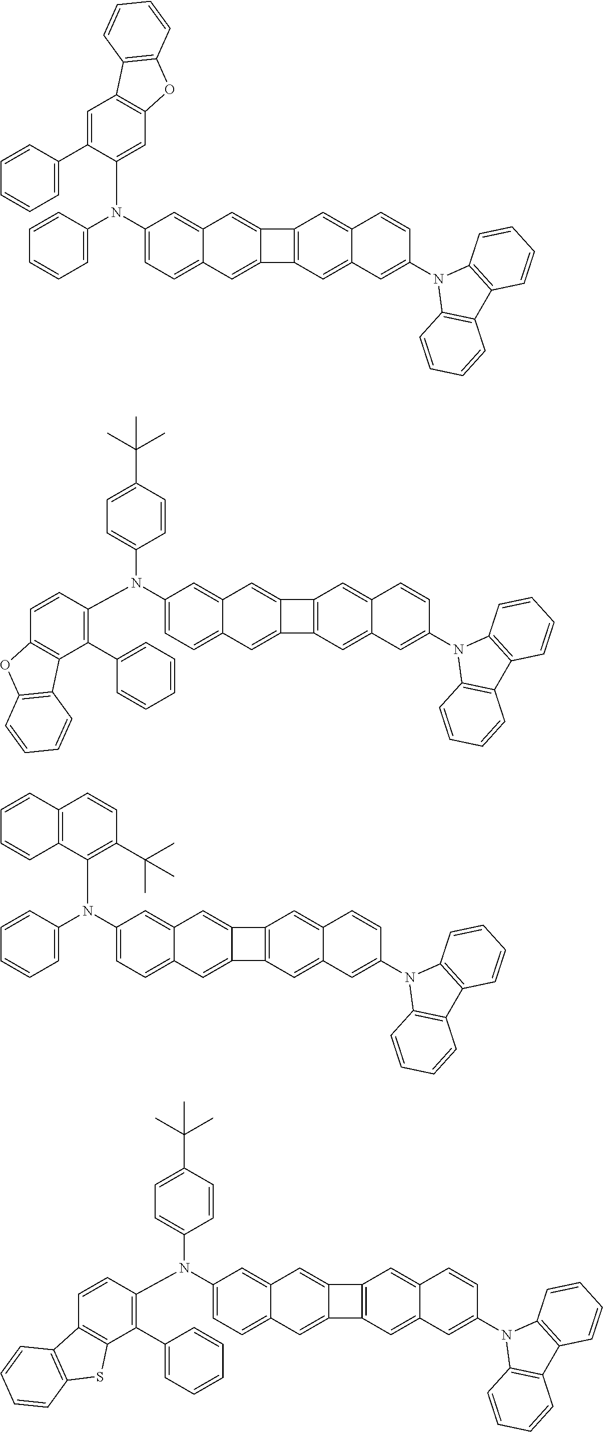

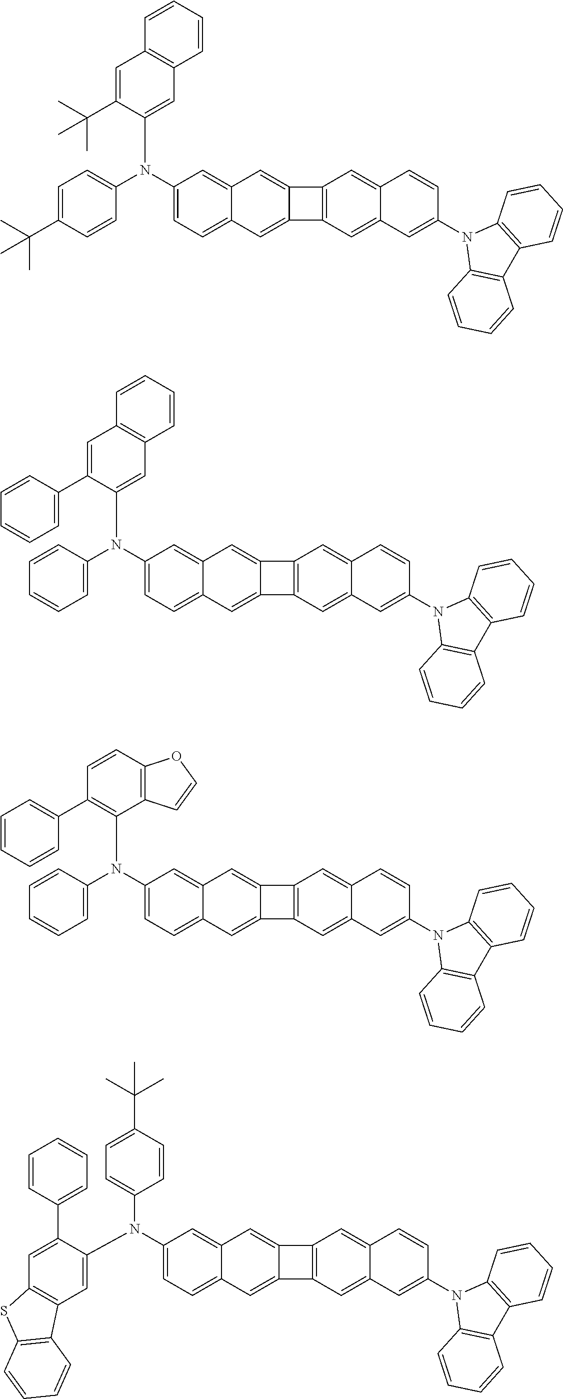 [SMILES: CC(C)(C)c1ccc(N(c2ccc3cc4c(cc3c2)-c2cc3ccc(-n5c6ccccc6c6ccccc65)cc3cc2-4)c2cc3ccccc3cc2C(C)(C)C)cc1 ; c1ccc(-c2cc3ccccc3cc2N(c2ccccc2)c2ccc3cc4c(cc3c2)-c2cc3ccc(-n5c6ccccc6c6ccccc65)cc3cc2-4)cc1 ; c1ccc(-c2ccc3occc3c2N(c2ccccc2)c2ccc3cc4c(cc3c2)-c2cc3ccc(-n5c6ccccc6c6ccccc65)cc3cc2-4)cc1 ; CC(C)(C)c1ccc(N(c2ccc3cc4c(cc3c2)-c2cc3ccc(-n5c6ccccc6c6ccccc65)cc3cc2-4)c2cc3c(cc2-c2ccccc2)sc2ccccc23)cc1]

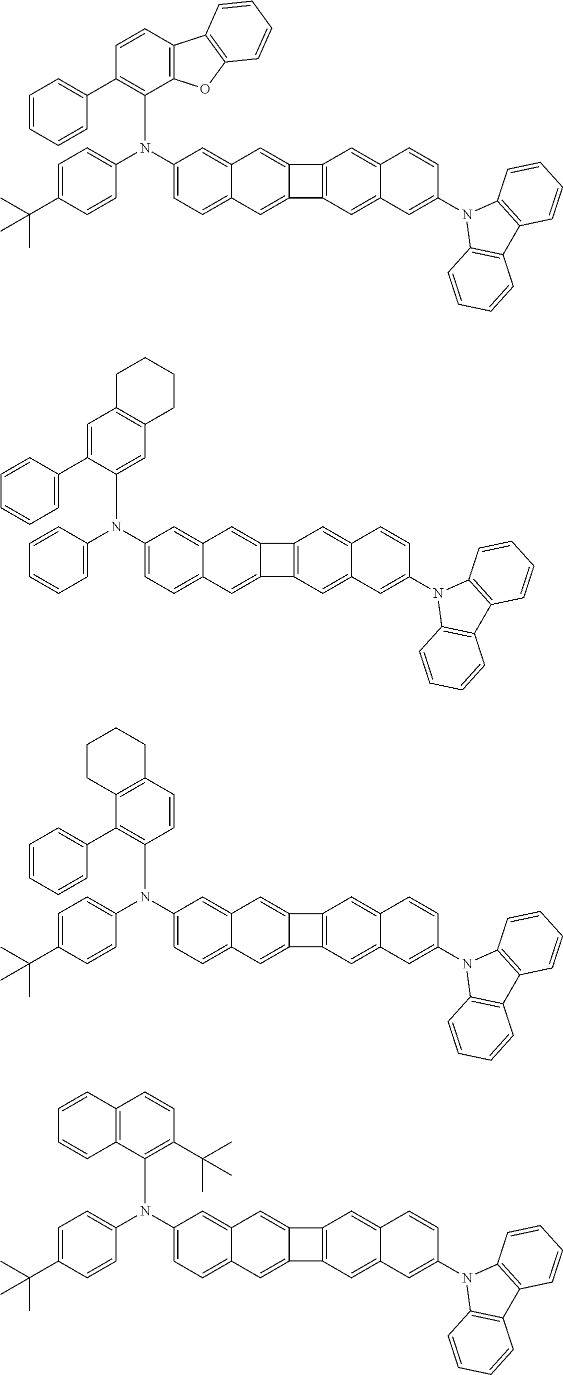 [SMILES: CC(C)(C)c1ccc(N(c2ccc3cc4c(cc3c2)-c2cc3ccc(-n5c6ccccc6c6ccccc65)cc3cc2-4)c2c(-c3ccccc3)ccc3c2oc2ccccc23)cc1 ; c1ccc(-c2cc3c(cc2N(c2ccccc2)c2ccc4cc5c(cc4c2)-c2cc4ccc(-n6c7ccccc7c7ccccc76)cc4cc2-5)CCCC3)cc1 ; CC(C)(C)c1ccc(N(c2ccc3cc4c(cc3c2)-c2cc3ccc(-n5c6ccccc6c6ccccc65)cc3cc2-4)c2ccc3c(c2-c2ccccc2)CCCC3)cc1 ; CC(C)(C)c1ccc(N(c2ccc3cc4c(cc3c2)-c2cc3ccc(-n5c6ccccc6c6ccccc65)cc3cc2-4)c2c(C(C)(C)C)ccc3ccccc23)cc1]

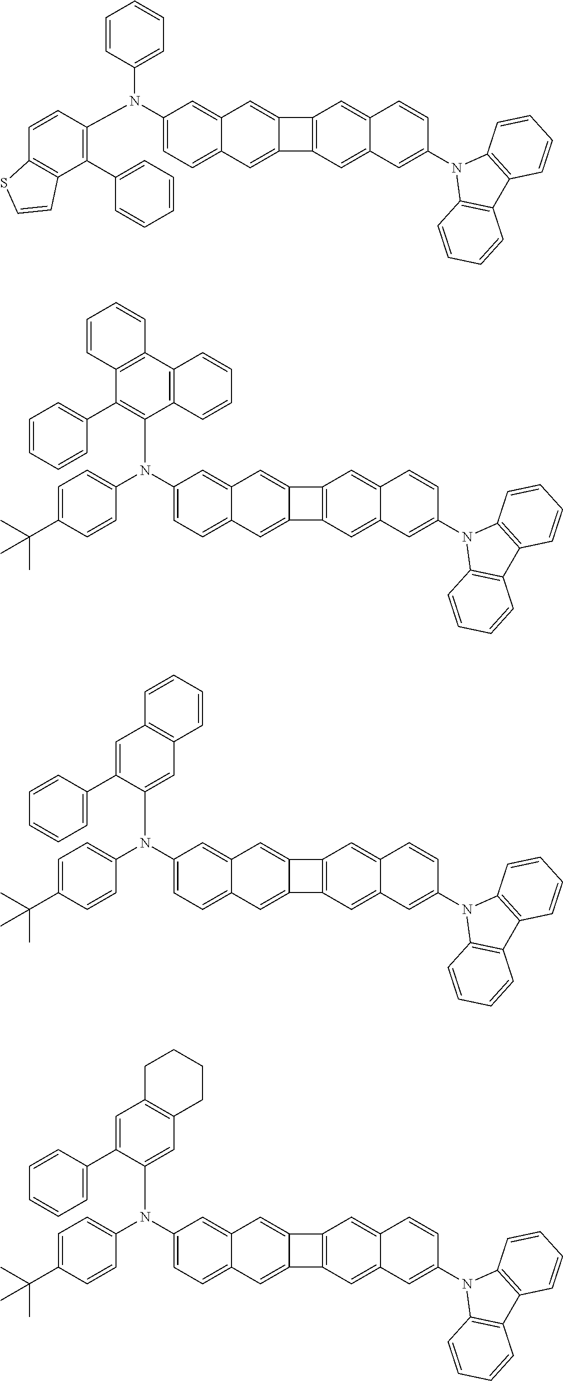 [SMILES: c1ccc(-c2c(N(c3ccccc3)c3ccc4cc5c(cc4c3)-c3cc4ccc(-n6c7ccccc7c7ccccc76)cc4cc3-5)ccc3sccc23)cc1 ; CC(C)(C)c1ccc(N(c2ccc3cc4c(cc3c2)-c2cc3ccc(-n5c6ccccc6c6ccccc65)cc3cc2-4)c2c(-c3ccccc3)c3ccccc3c3ccccc23)cc1 ; CC(C)(C)c1ccc(N(c2ccc3cc4c(cc3c2)-c2cc3ccc(-n5c6ccccc6c6ccccc65)cc3cc2-4)c2cc3ccccc3cc2-c2ccccc2)cc1 ; CC(C)(C)c1ccc(N(c2ccc3cc4c(cc3c2)-c2cc3ccc(-n5c6ccccc6c6ccccc65)cc3cc2-4)c2cc3c(cc2-c2ccccc2)CCCC3)cc1]

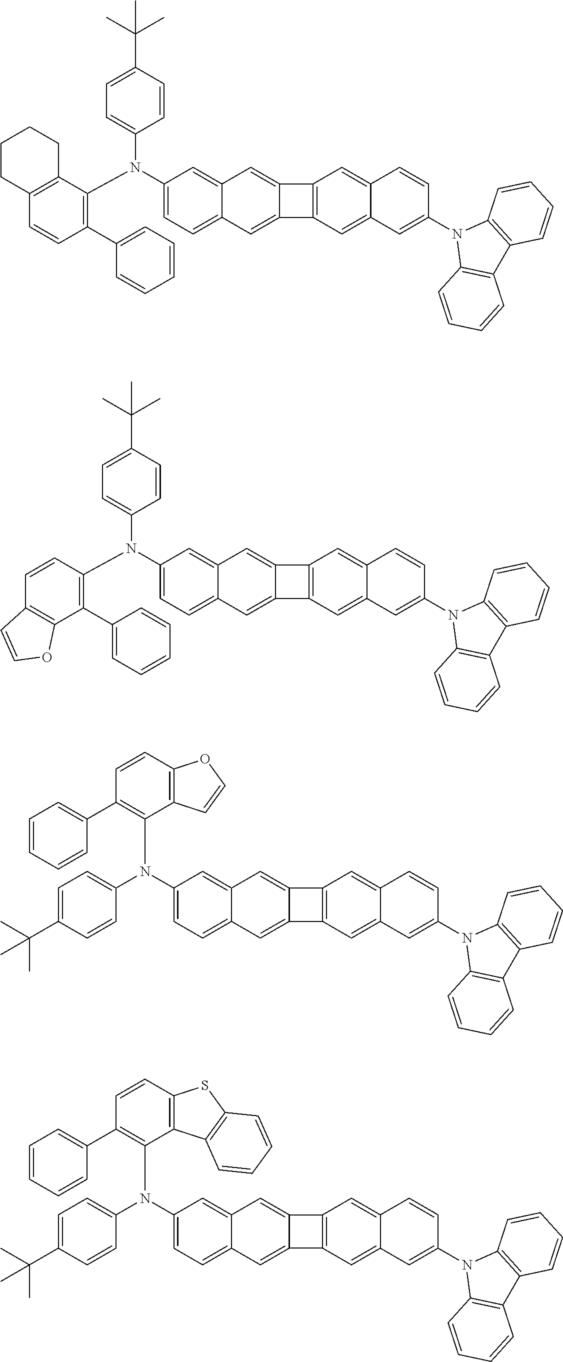 [SMILES: CC(C)(C)c1ccc(N(c2ccc3cc4c(cc3c2)-c2cc3ccc(-n5c6ccccc6c6ccccc65)cc3cc2-4)c2c(-c3ccccc3)ccc3c2CCCC3)cc1 ; CC(C)(C)c1ccc(N(c2ccc3cc4c(cc3c2)-c2cc3ccc(-n5c6ccccc6c6ccccc65)cc3cc2-4)c2ccc3ccoc3c2-c2ccccc2)cc1 ; CC(C)(C)c1ccc(N(c2ccc3cc4c(cc3c2)-c2cc3ccc(-n5c6ccccc6c6ccccc65)cc3cc2-4)c2c(-c3ccccc3)ccc3occc23)cc1 ; CC(C)(C)c1ccc(N(c2ccc3cc4c(cc3c2)-c2cc3ccc(-n5c6ccccc6c6ccccc65)cc3cc2-4)c2c(-c3ccccc3)ccc3sc4ccccc4c23)cc1]

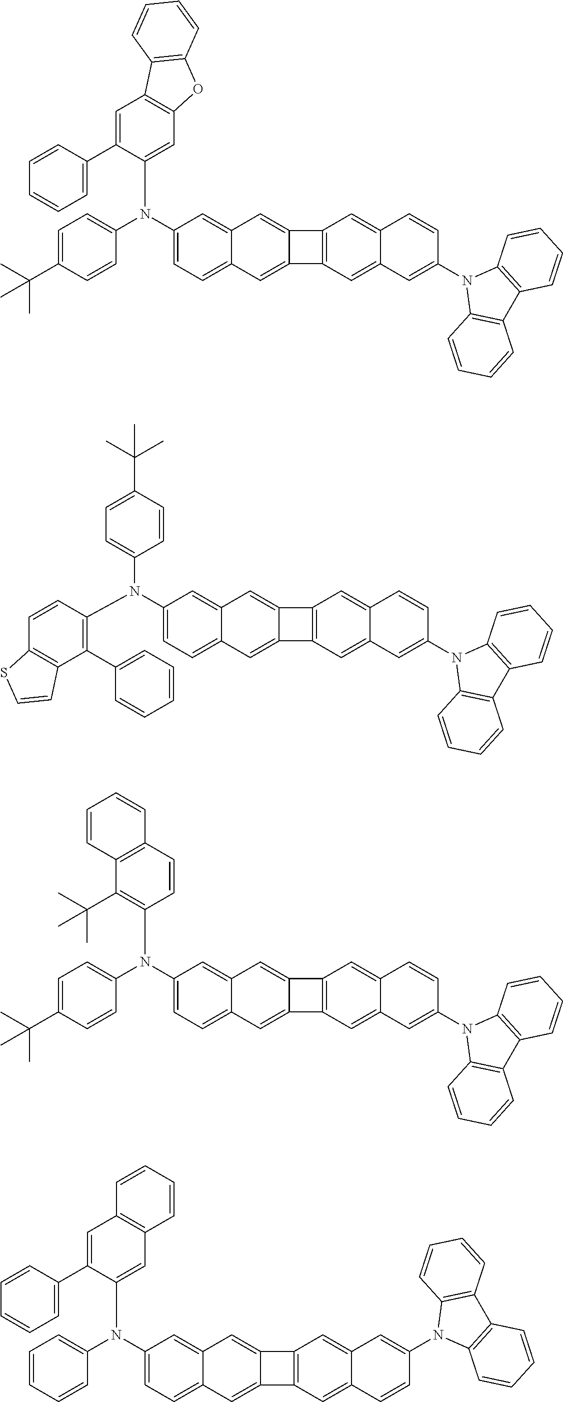 [SMILES: CC(C)(C)c1ccc(N(c2ccc3cc4c(cc3c2)-c2cc3ccc(-n5c6ccccc6c6ccccc65)cc3cc2-4)c2cc3oc4ccccc4c3cc2-c2ccccc2)cc1 ; CC(C)(C)c1ccc(N(c2ccc3cc4c(cc3c2)-c2cc3ccc(-n5c6ccccc6c6ccccc65)cc3cc2-4)c2ccc3sccc3c2-c2ccccc2)cc1 ; CC(C)(C)c1ccc(N(c2ccc3cc4c(cc3c2)-c2cc3ccc(-n5c6ccccc6c6ccccc65)cc3cc2-4)c2ccc3ccccc3c2C(C)(C)C)cc1 ; c1ccc(-c2cc3ccccc3cc2N(c2ccccc2)c2ccc3cc4c(cc3c2)-c2cc3cc(-n5c6ccccc6c6ccccc65)ccc3cc2-4)cc1]

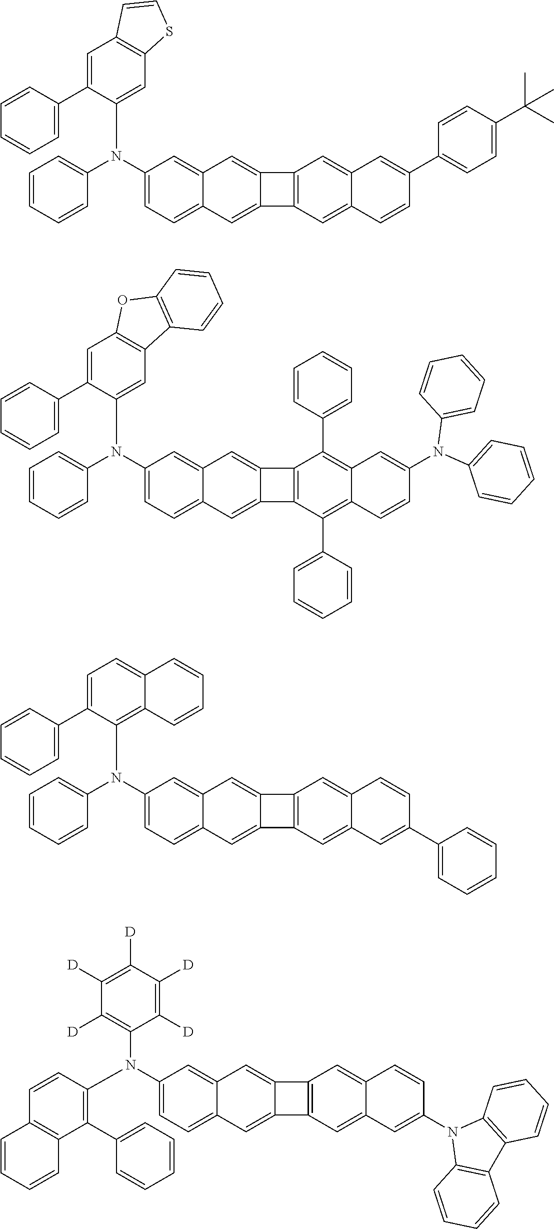

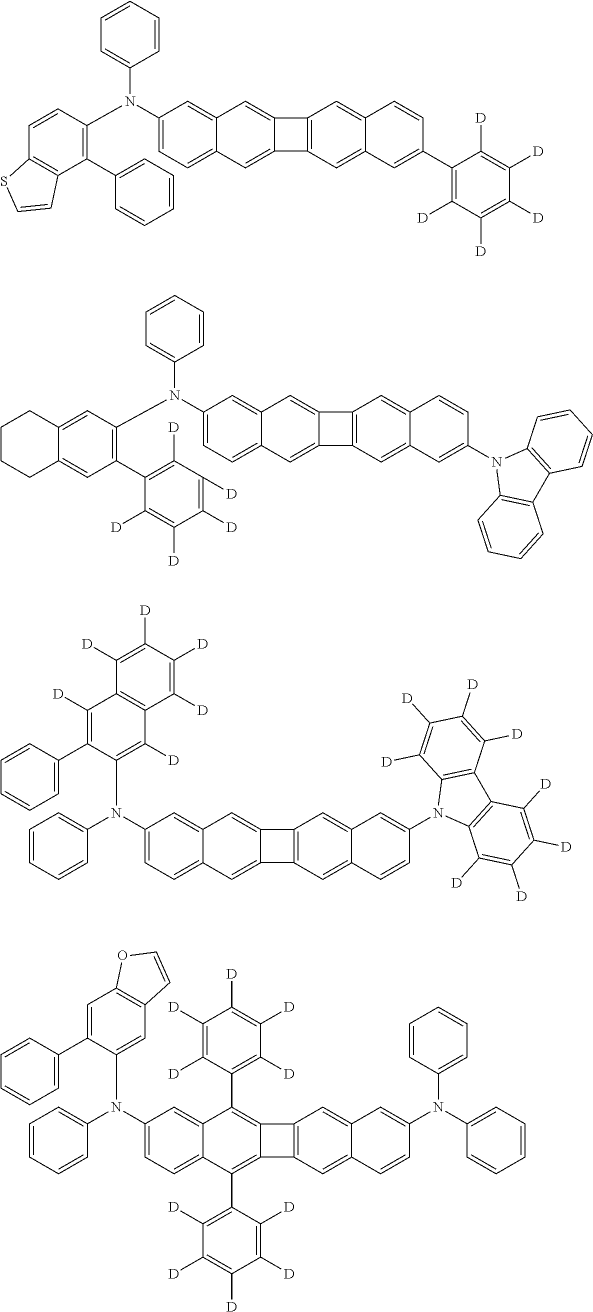

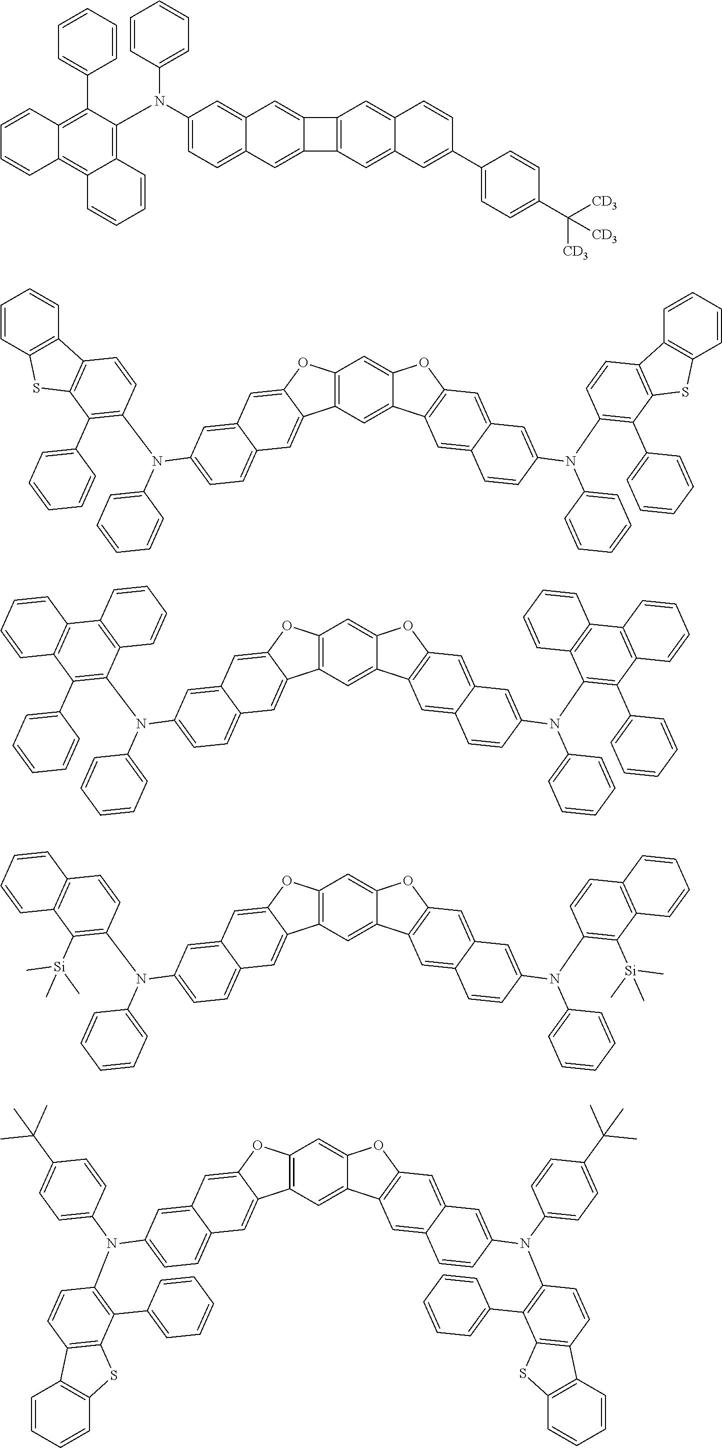

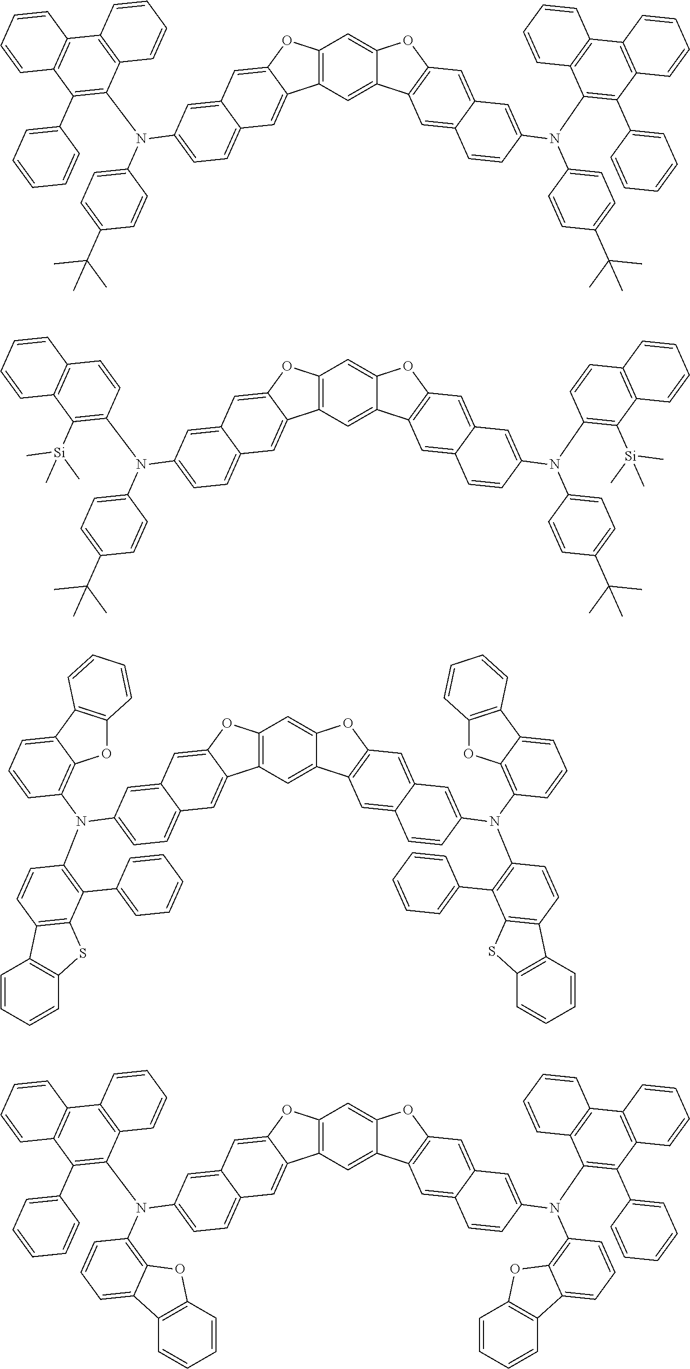

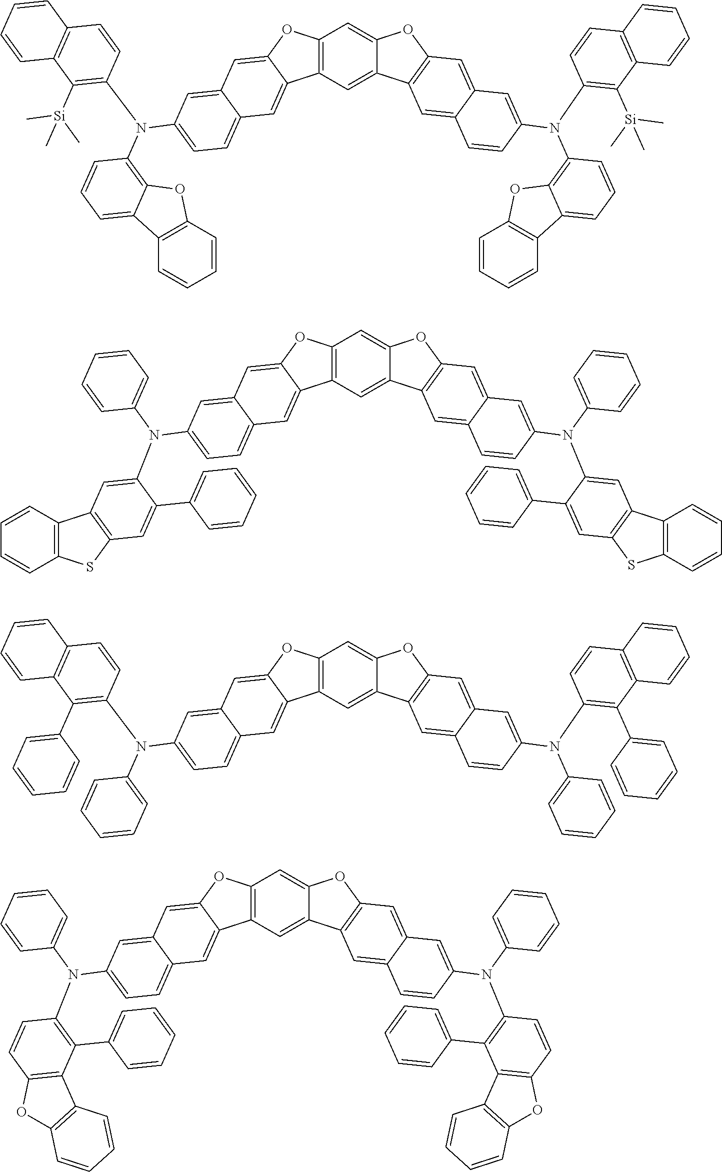

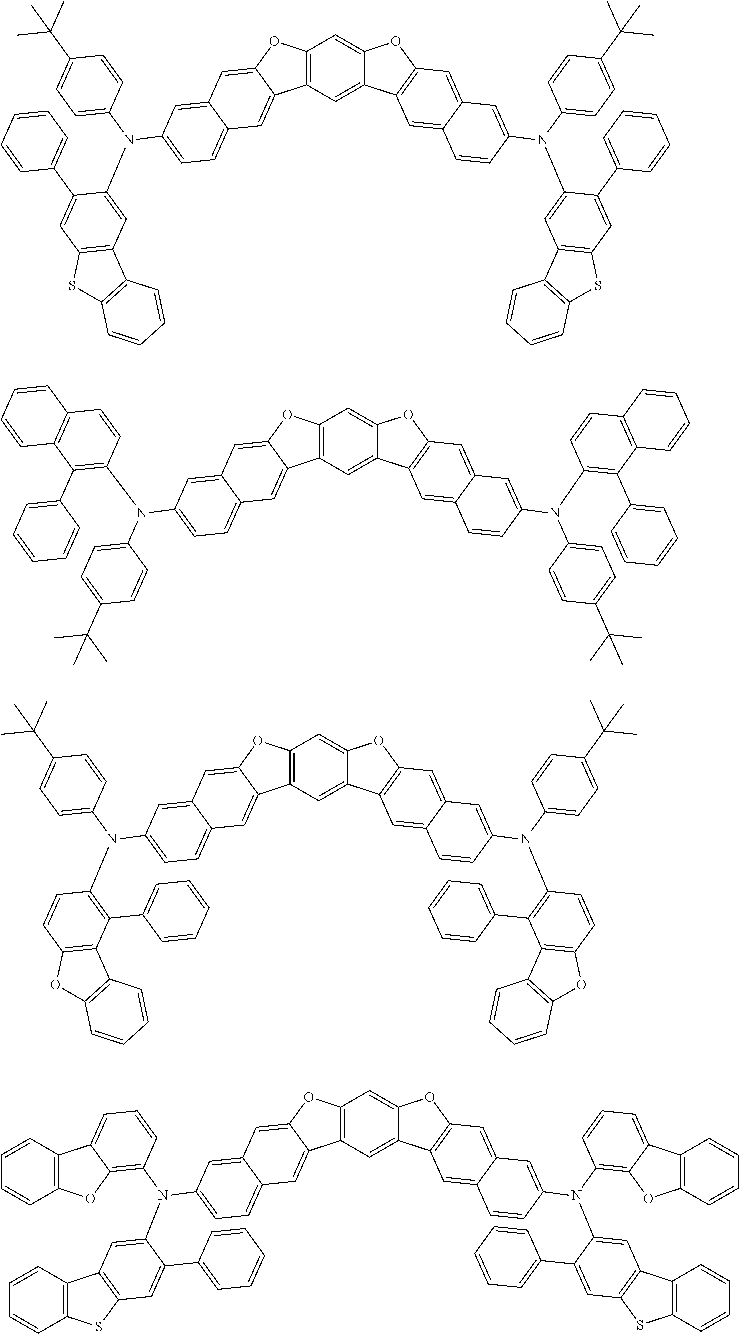

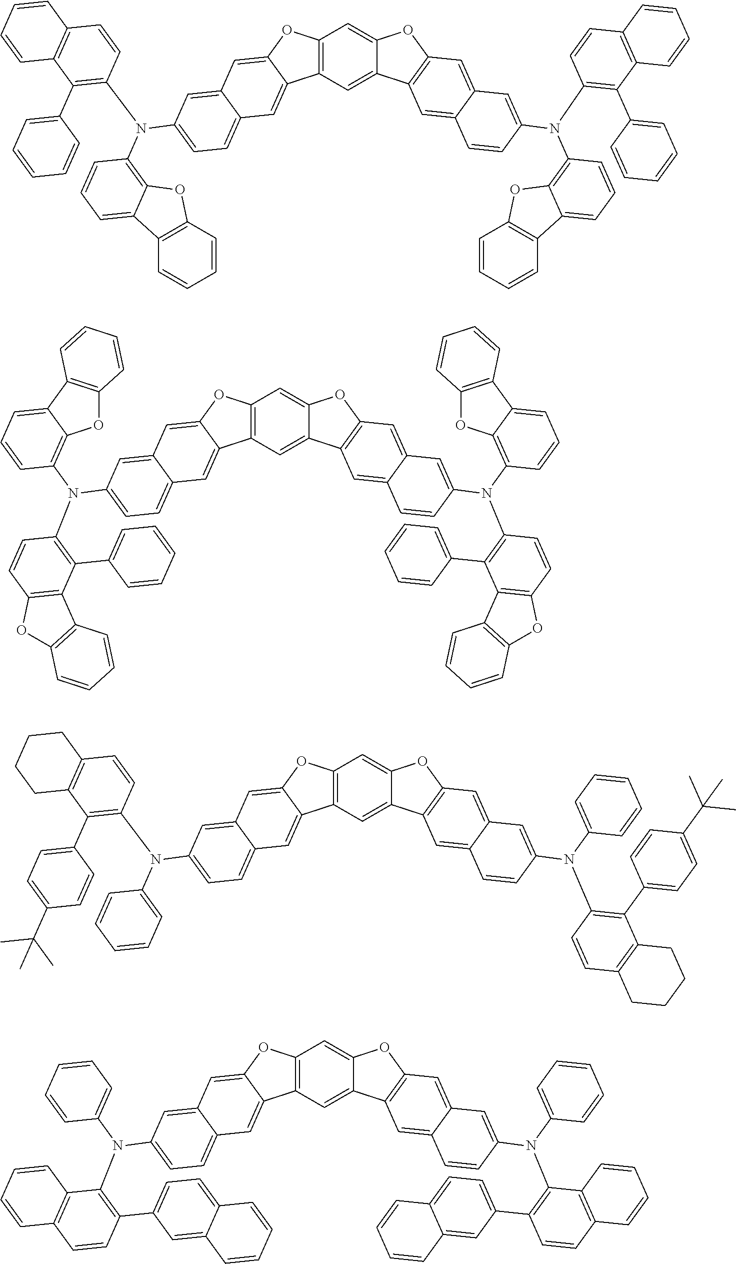

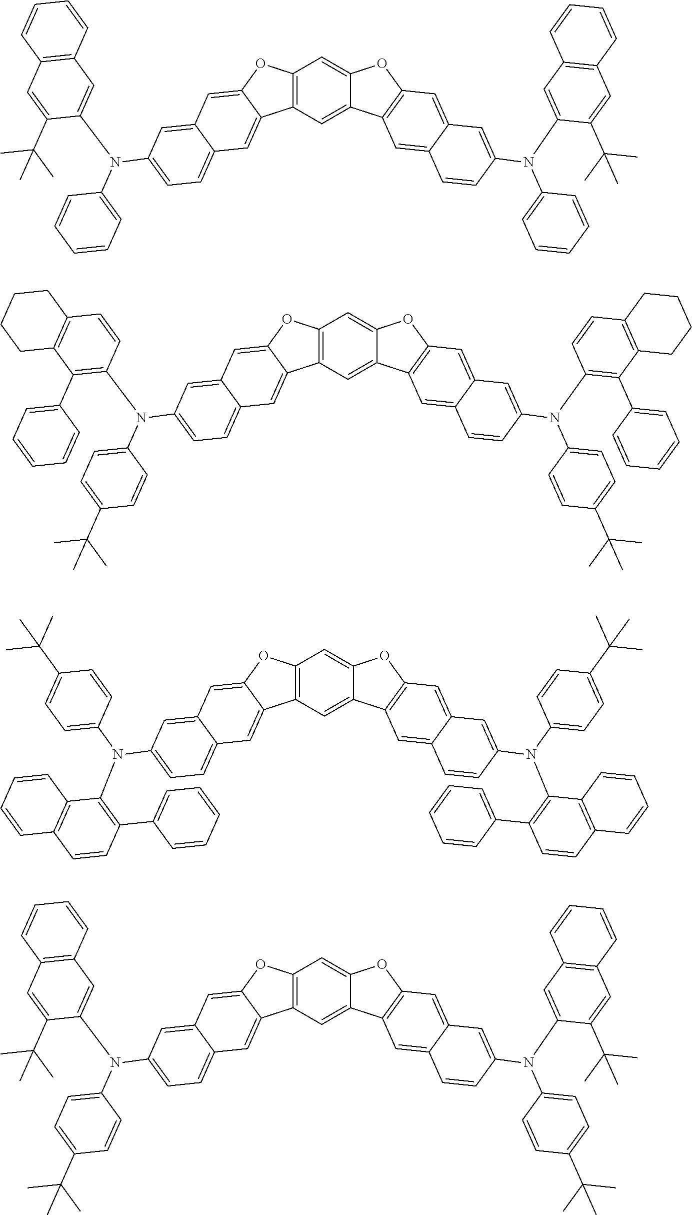

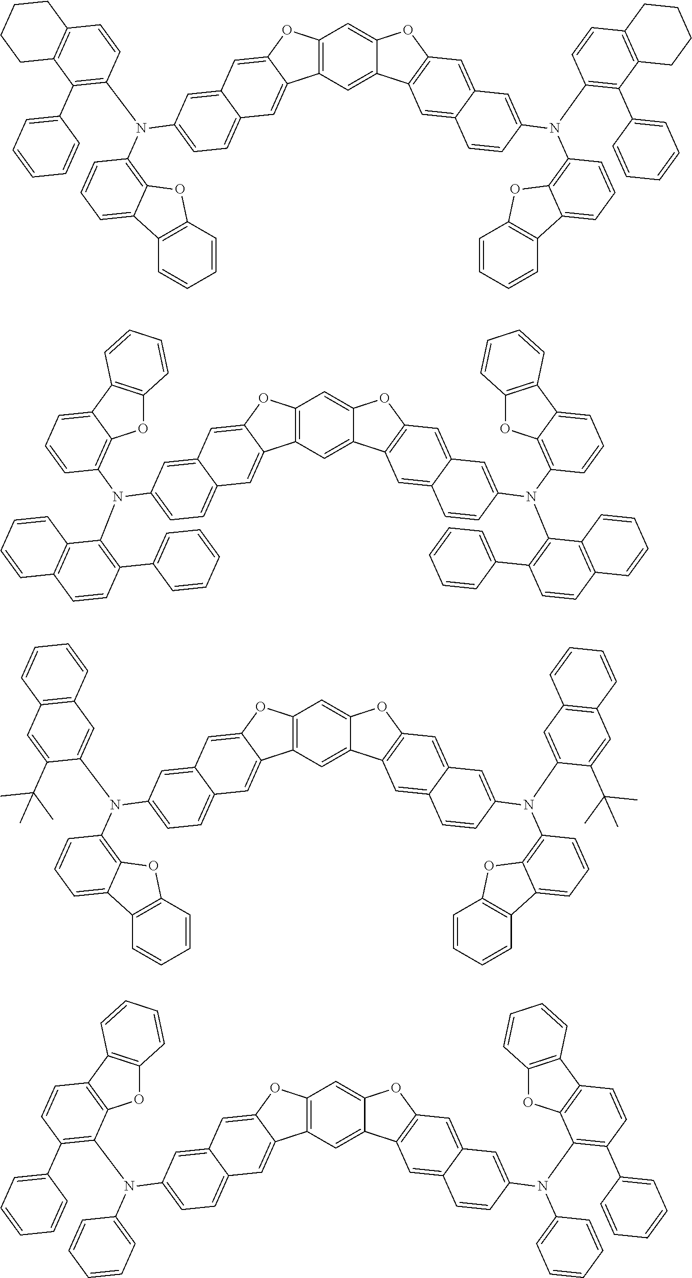

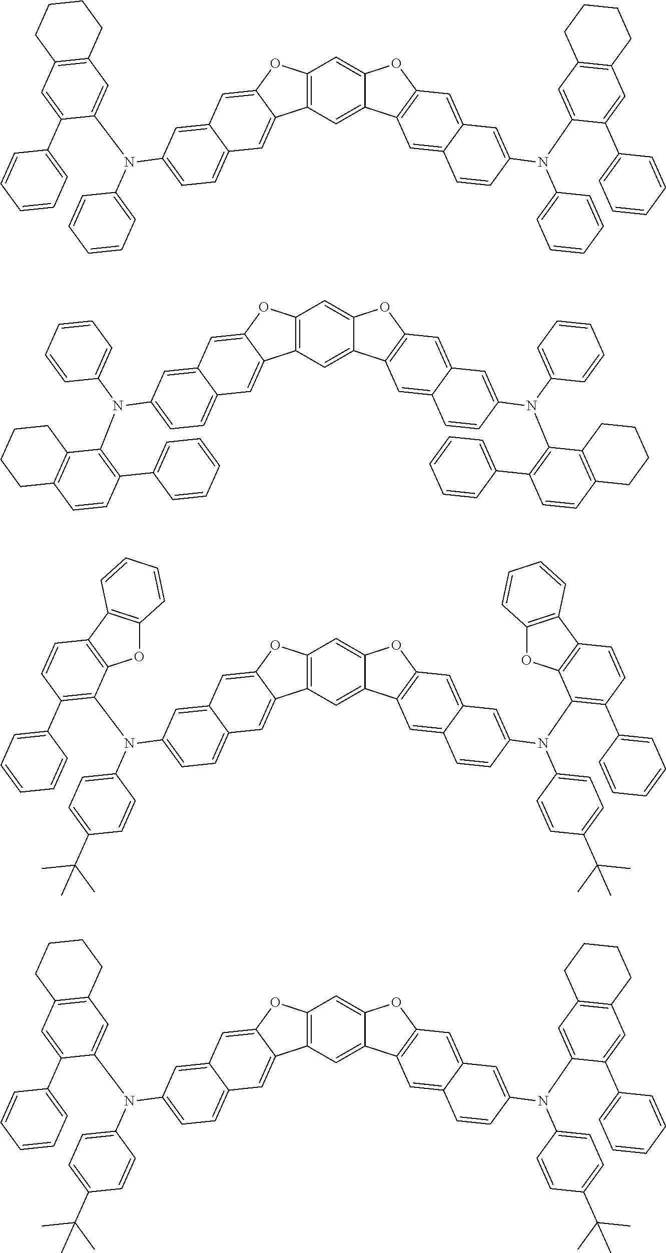

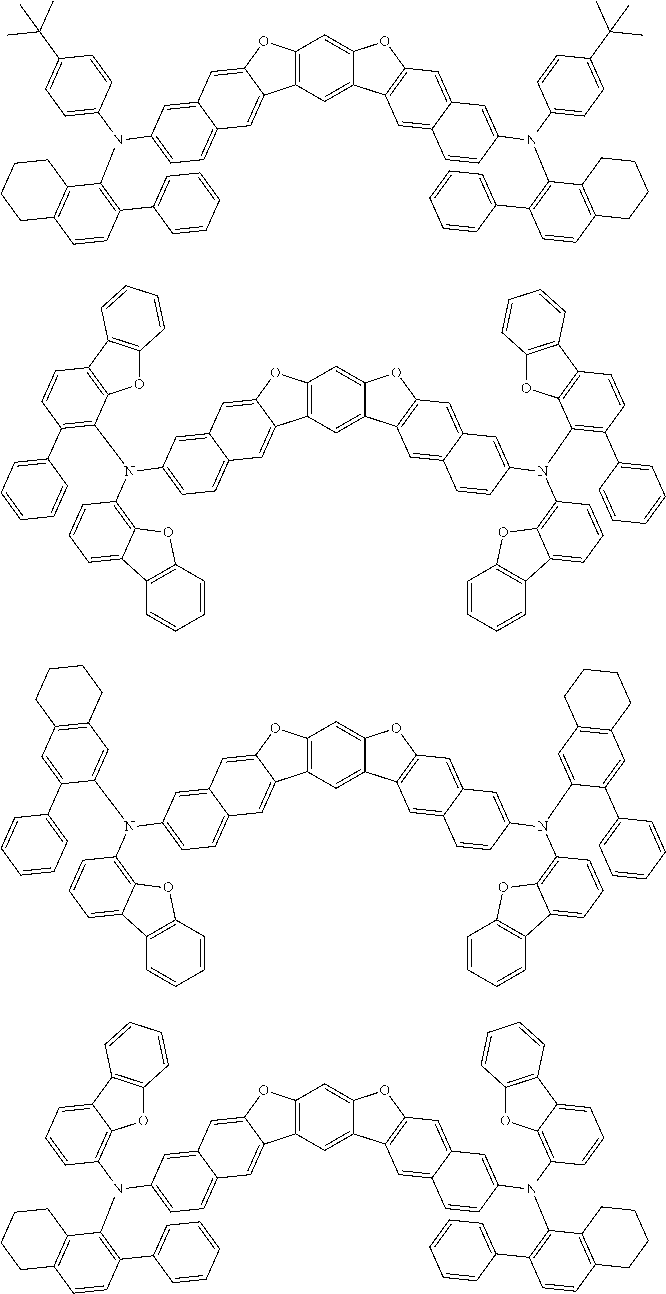

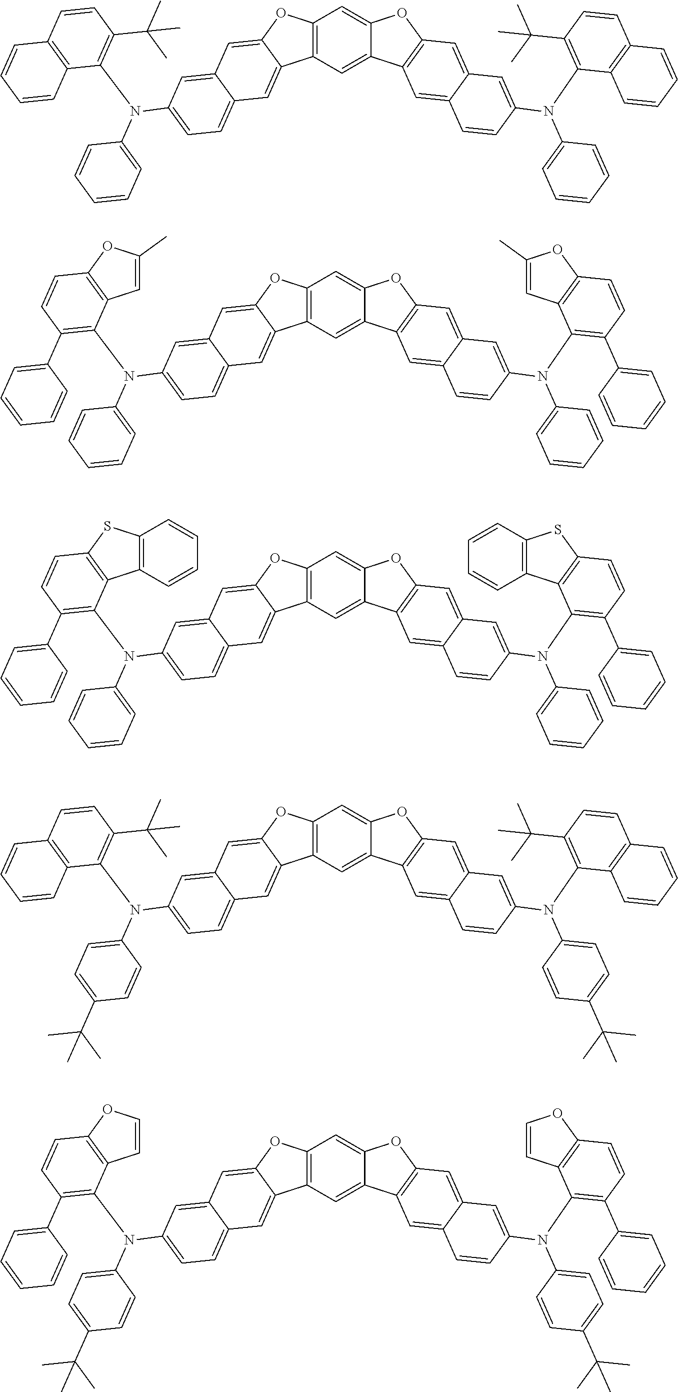

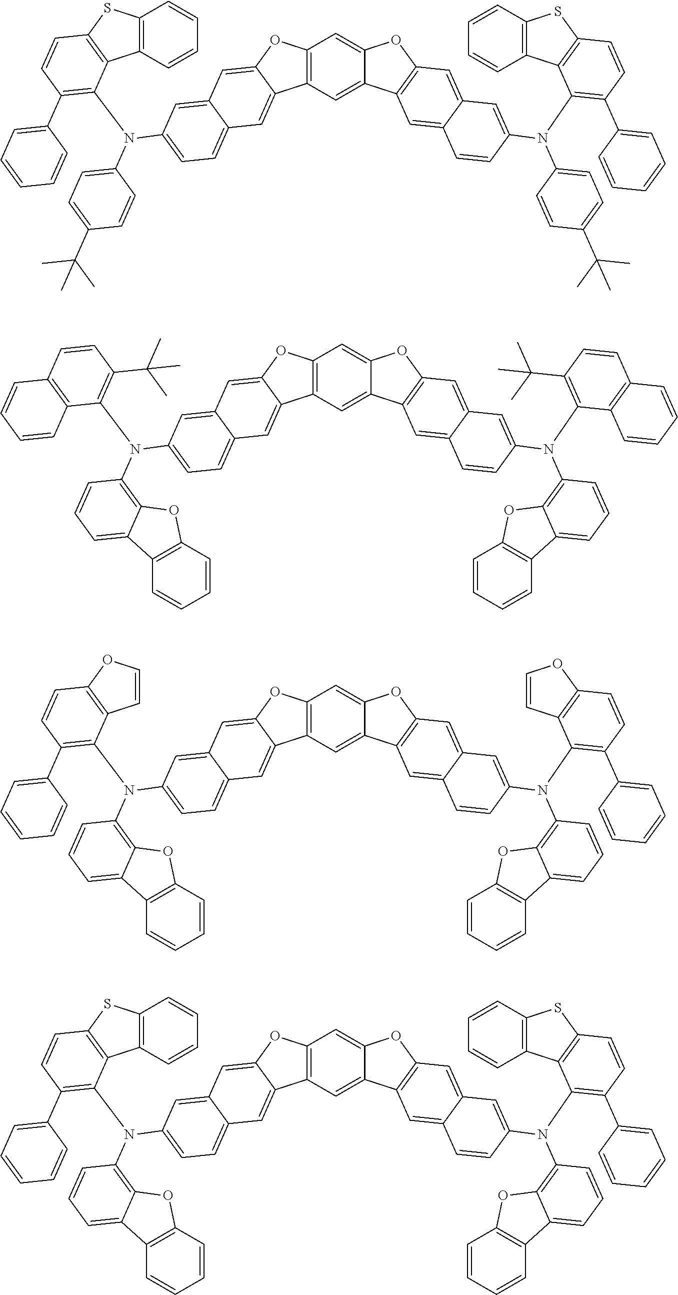

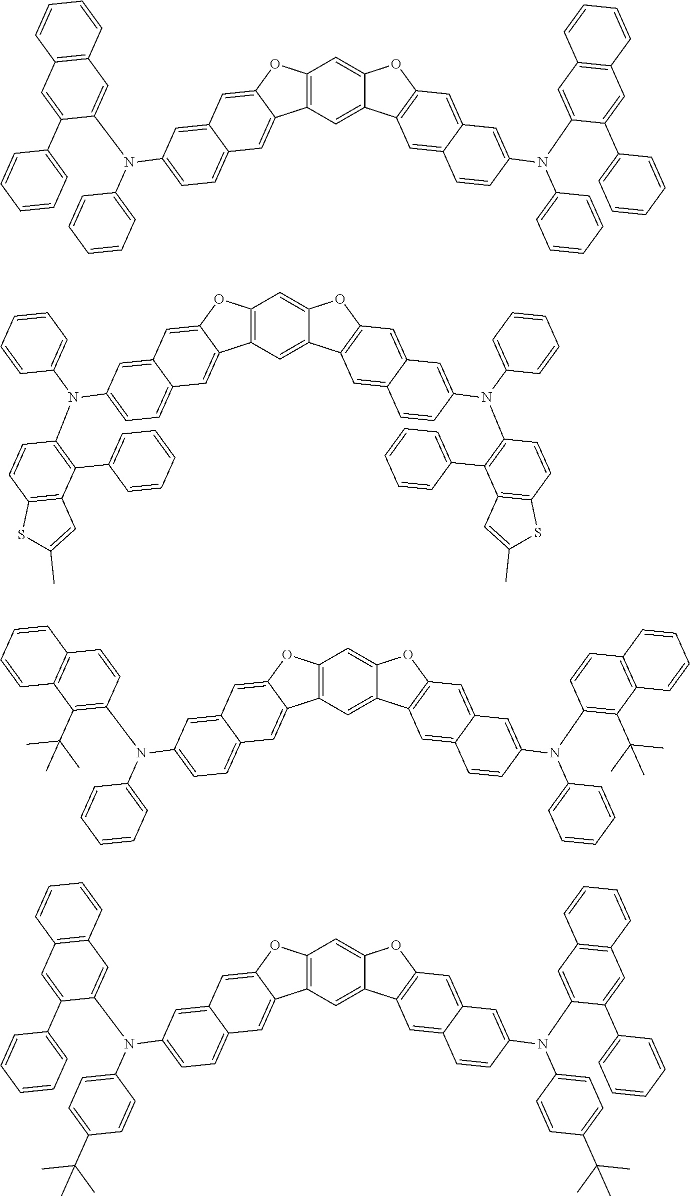

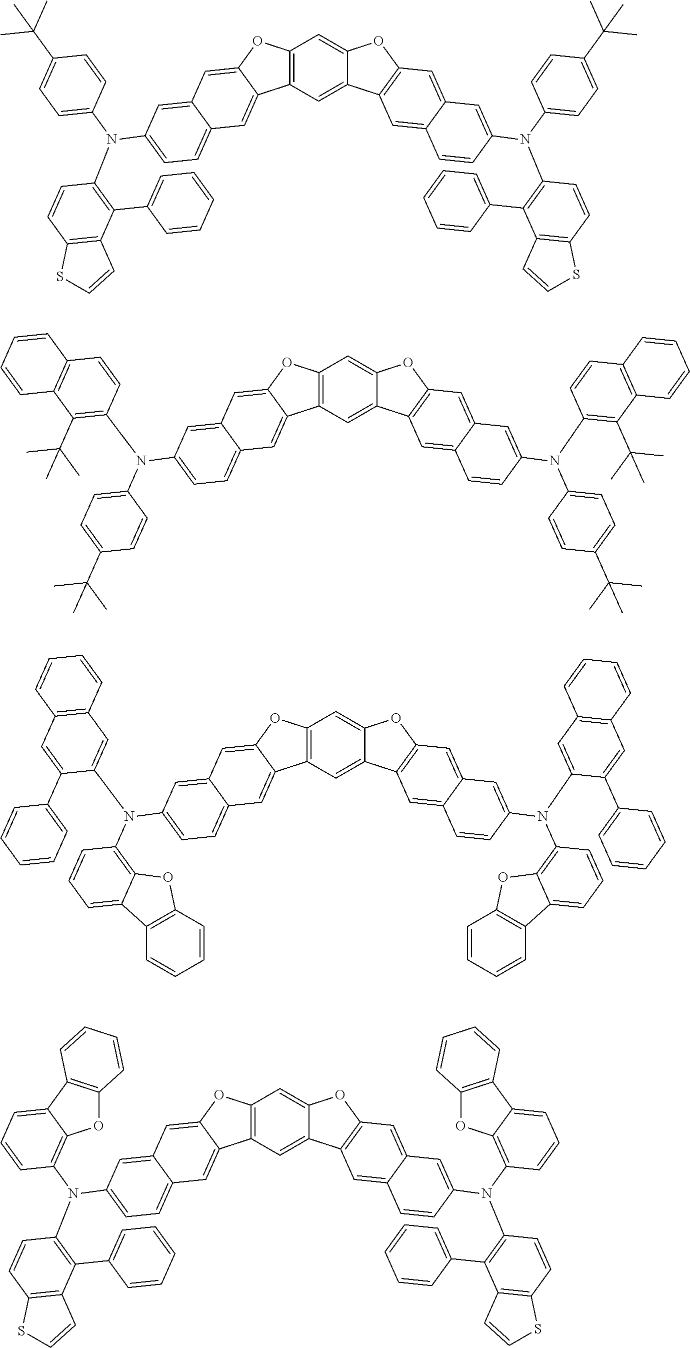

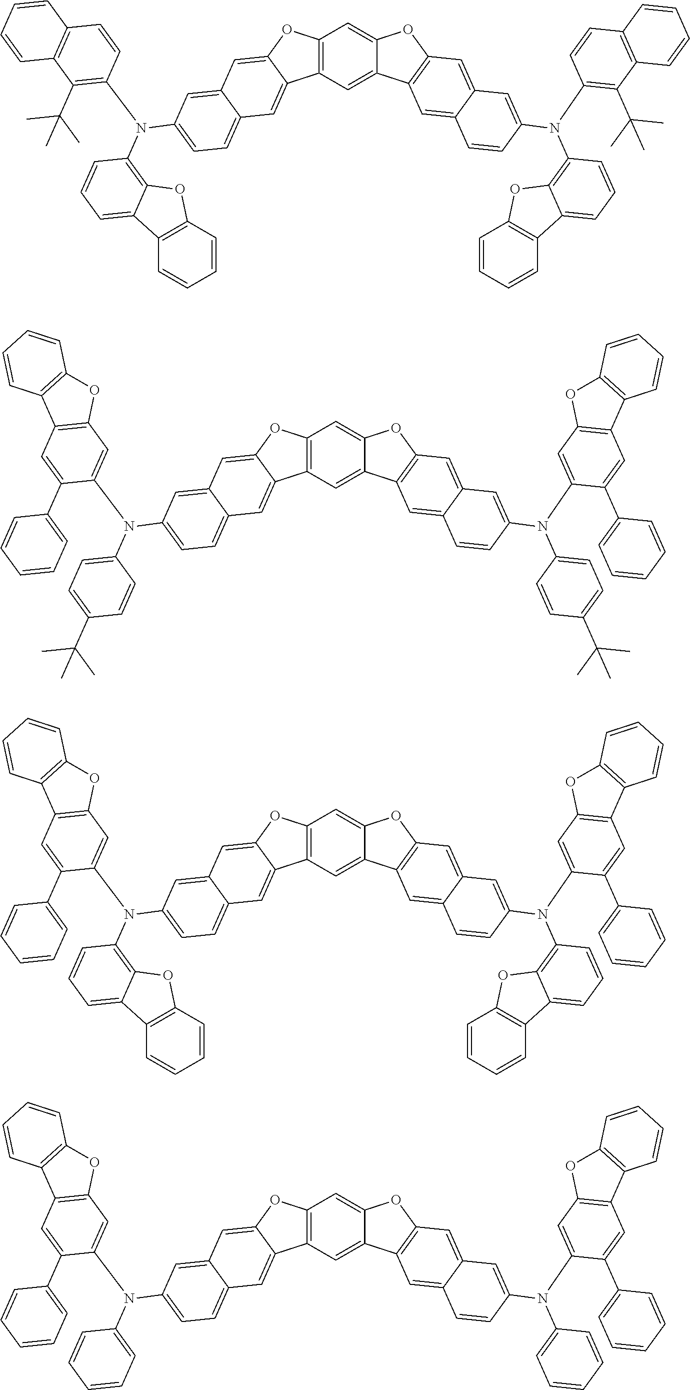

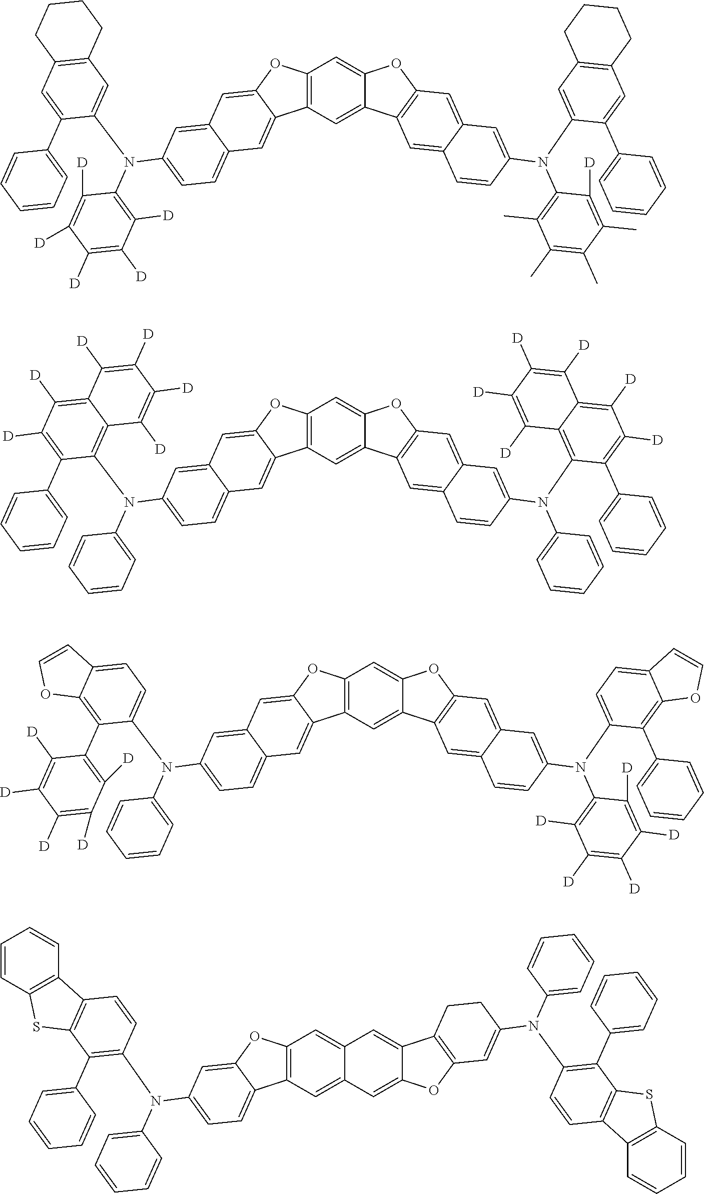

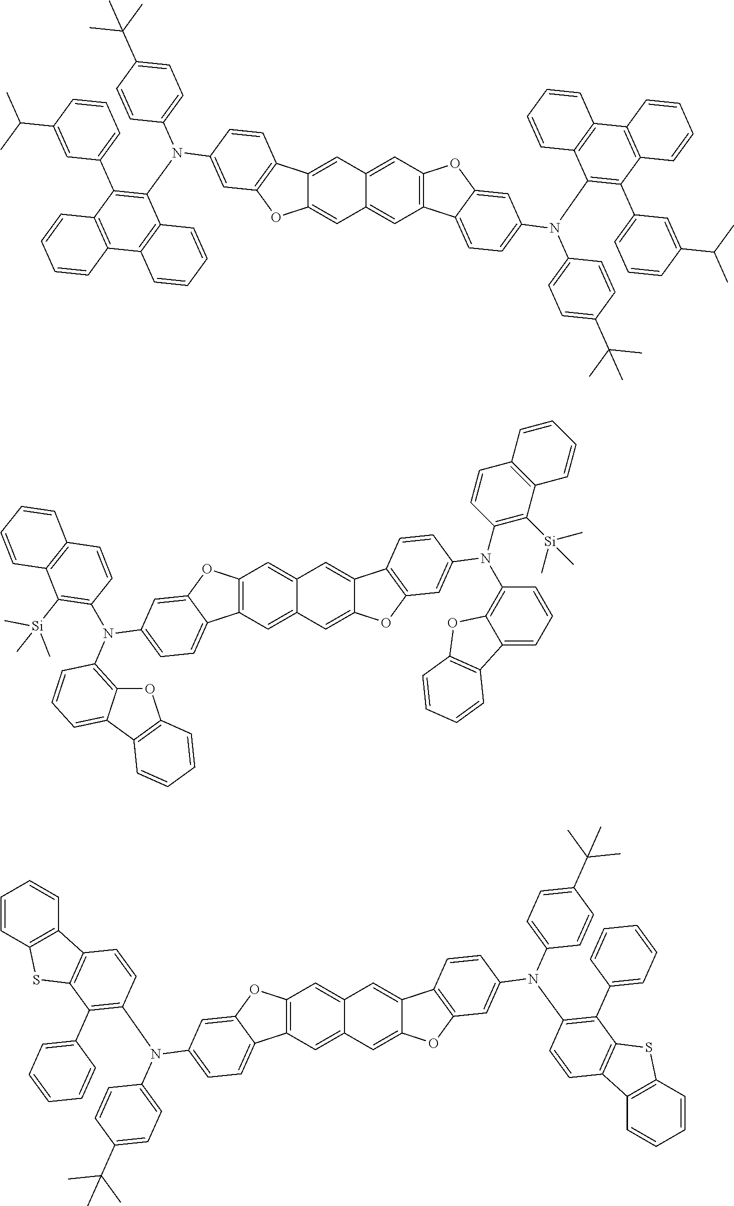 [SMILES: CC(C)c1cccc(-c2c(N(c3ccc(C(C)(C)C)cc3)c3ccc4c(c3)oc3cc5cc6c(cc5cc34)oc3cc(N(c4ccc(C(C)(C)C)cc4)c4c(-c5cccc(C(C)C)c5)c5ccccc5c5ccccc45)ccc36)c3ccccc3c3ccccc23)c1 ; C[Si](C)(C)c1c(N(c2ccc3c(c2)oc2cc4cc5c(cc4cc23)oc2cc(N(c3ccc4ccccc4c3[Si](C)(C)C)c3cccc4c3oc3ccccc34)ccc25)c2cccc3c2oc2ccccc23)ccc2ccccc12 ; CC(C)(C)c1ccc(N(c2ccc3c(c2)oc2cc4cc5c(cc4cc23)oc2cc(N(c3ccc(C(C)(C)C)cc3)c3ccc4c(sc6ccccc64)c3-c3ccccc3)ccc25)c2ccc3c(sc4ccccc43)c2-c2ccccc2)cc1]

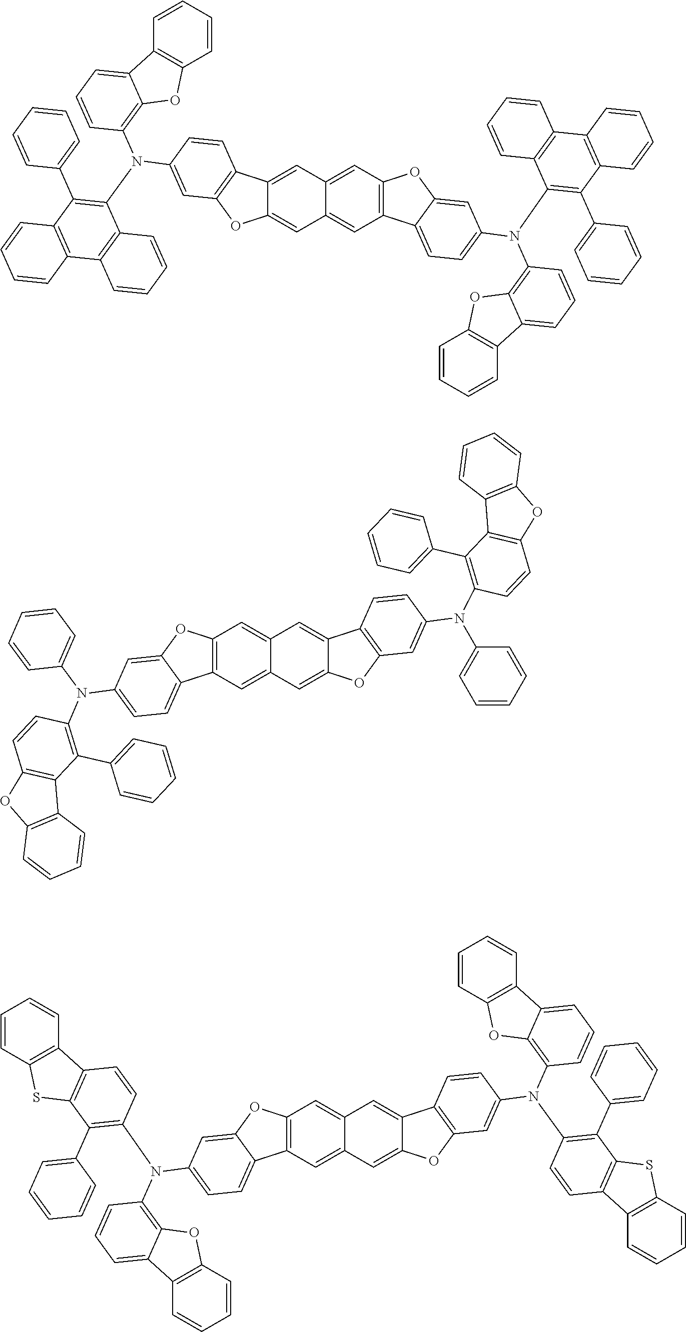

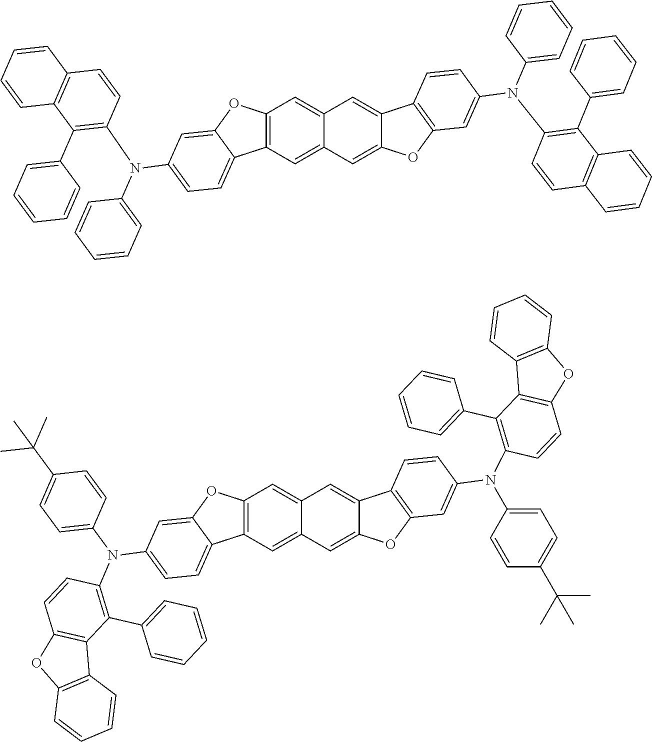 [SMILES: c1ccc(-c2c(N(c3ccccc3)c3ccc4c(c3)oc3cc5cc6c(cc5cc34)oc3cc(N(c4ccccc4)c4ccc5ccccc5c4-c4ccccc4)ccc36)ccc3ccccc23)cc1 ; CC(C)(C)c1ccc(N(c2ccc3c(c2)oc2cc4cc5c(cc4cc23)oc2cc(N(c3ccc(C(C)(C)C)cc3)c3ccc4oc6ccccc6c4c3-c3ccccc3)ccc25)c2ccc3oc4ccccc4c3c2-c2ccccc2)cc1]

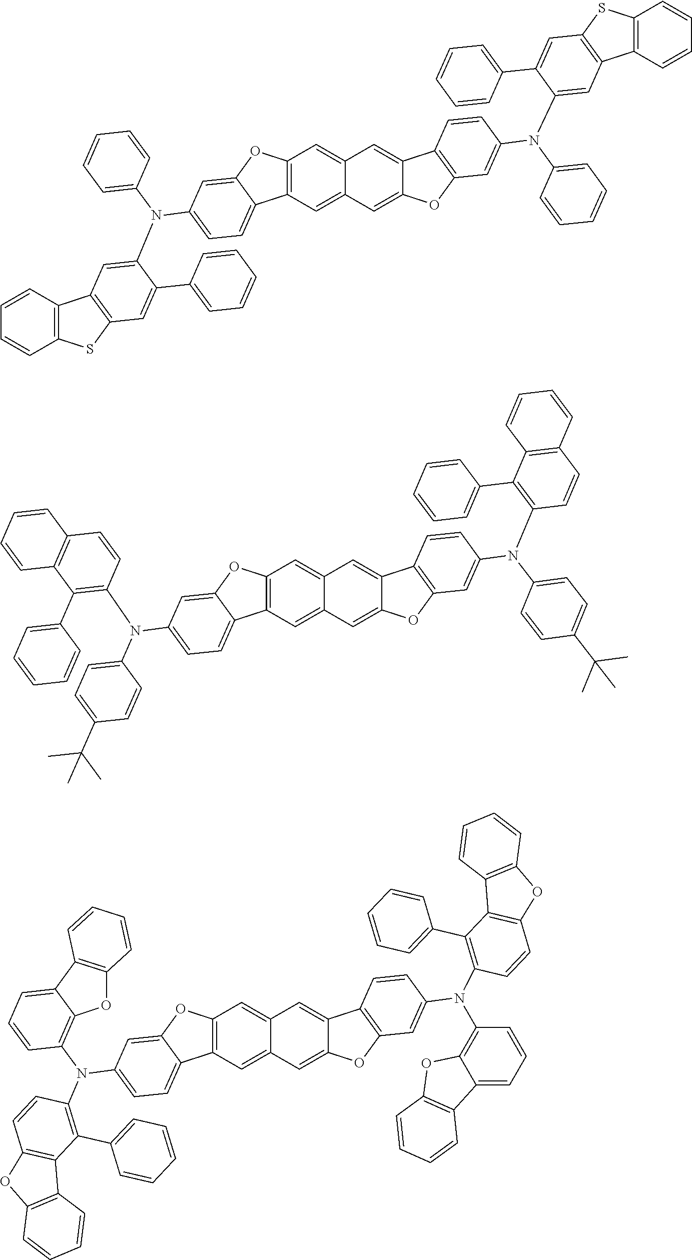 [SMILES: c1ccc(-c2cc3sc4ccccc4c3cc2N(c2ccccc2)c2ccc3c(c2)oc2cc4cc5c(cc4cc23)oc2cc(N(c3ccccc3)c3cc4c(cc3-c3ccccc3)sc3ccccc34)ccc25)cc1 ; CC(C)(C)c1ccc(N(c2ccc3c(c2)oc2cc4cc5c(cc4cc23)oc2cc(N(c3ccc(C(C)(C)C)cc3)c3ccc4ccccc4c3-c3ccccc3)ccc25)c2ccc3ccccc3c2-c2ccccc2)cc1 ; c1ccc(-c2c(N(c3ccc4c(c3)oc3cc5cc6c(cc5cc34)oc3cc(N(c4ccc5oc7ccccc7c5c4-c4ccccc4)c4cccc5c4oc4ccccc45)ccc36)c3cccc4c3oc3ccccc34)ccc3oc4ccccc4c23)cc1]

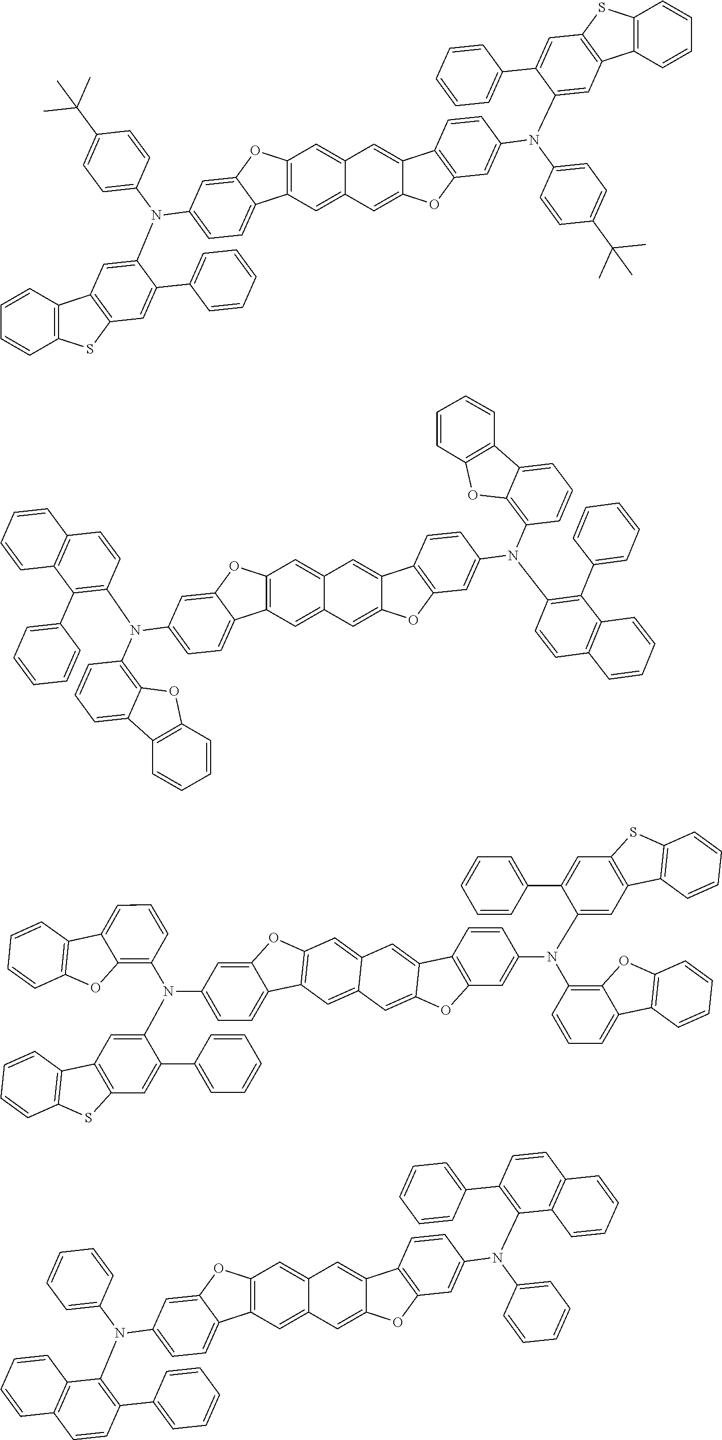

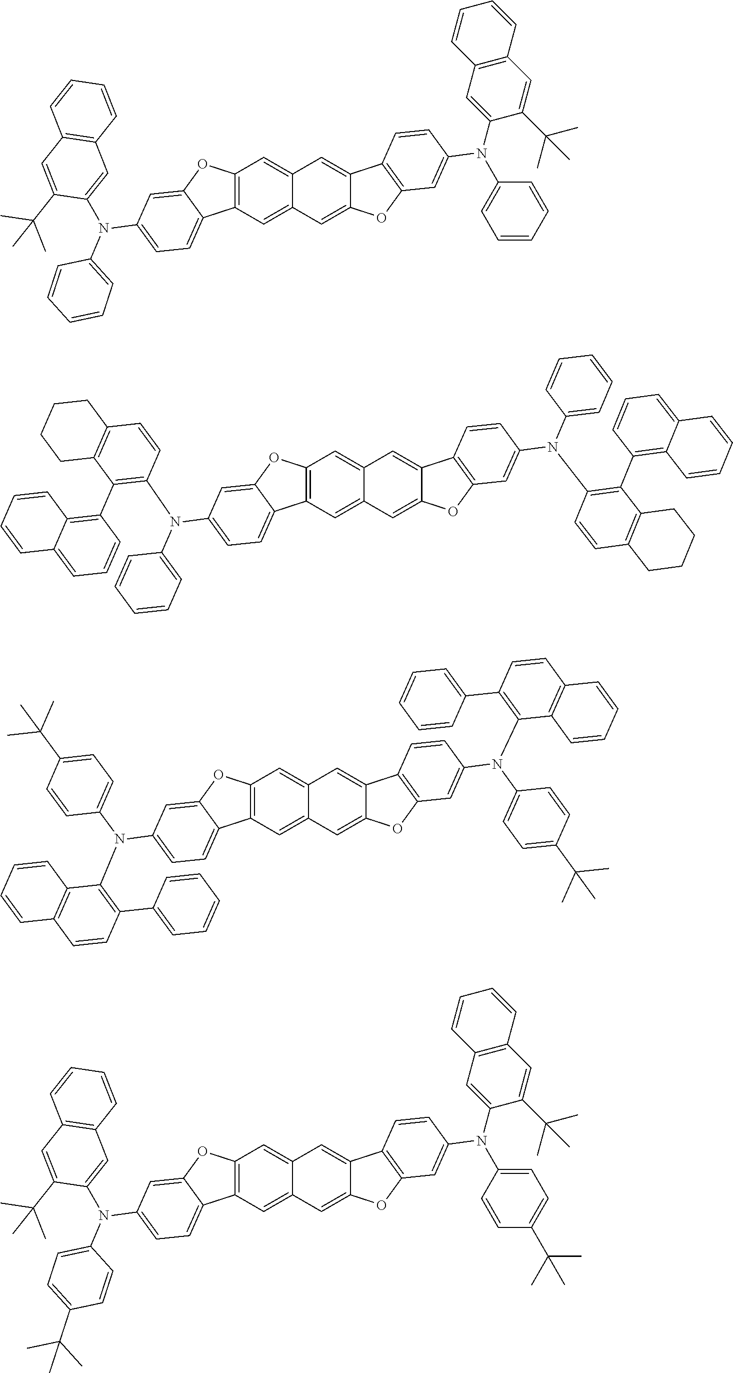

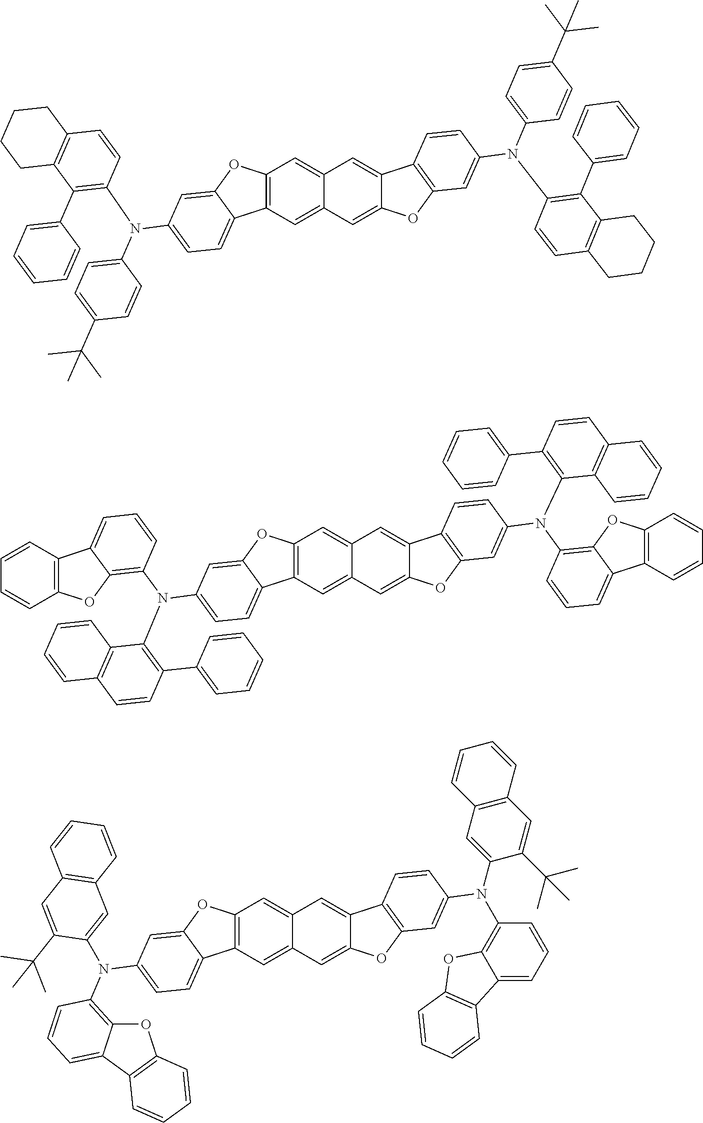 [SMILES: CC(C)(C)c1ccc(N(c2ccc3c(c2)oc2cc4cc5c(cc4cc23)oc2cc(N(c3ccc(C(C)(C)C)cc3)c3ccc4c(c3-c3ccccc3)CCCC4)ccc25)c2ccc3c(c2-c2ccccc2)CCCC3)cc1 ; c1ccc(-c2ccc3ccccc3c2N(c2ccc3c(c2)oc2cc4cc5c(cc4cc23)oc2cc(N(c3c(-c4ccccc4)ccc4ccccc34)c3cccc4c3oc3ccccc34)ccc25)c2cccc3c2oc2ccccc23)cc1 ; CC(C)(C)c1cc2ccccc2cc1N(c1ccc2c(c1)oc1cc3cc4c(cc3cc12)oc1cc(N(c2cc3ccccc3cc2C(C)(C)C)c2cccc3c2oc2ccccc23)ccc14)c1cccc2c1oc1ccccc12]

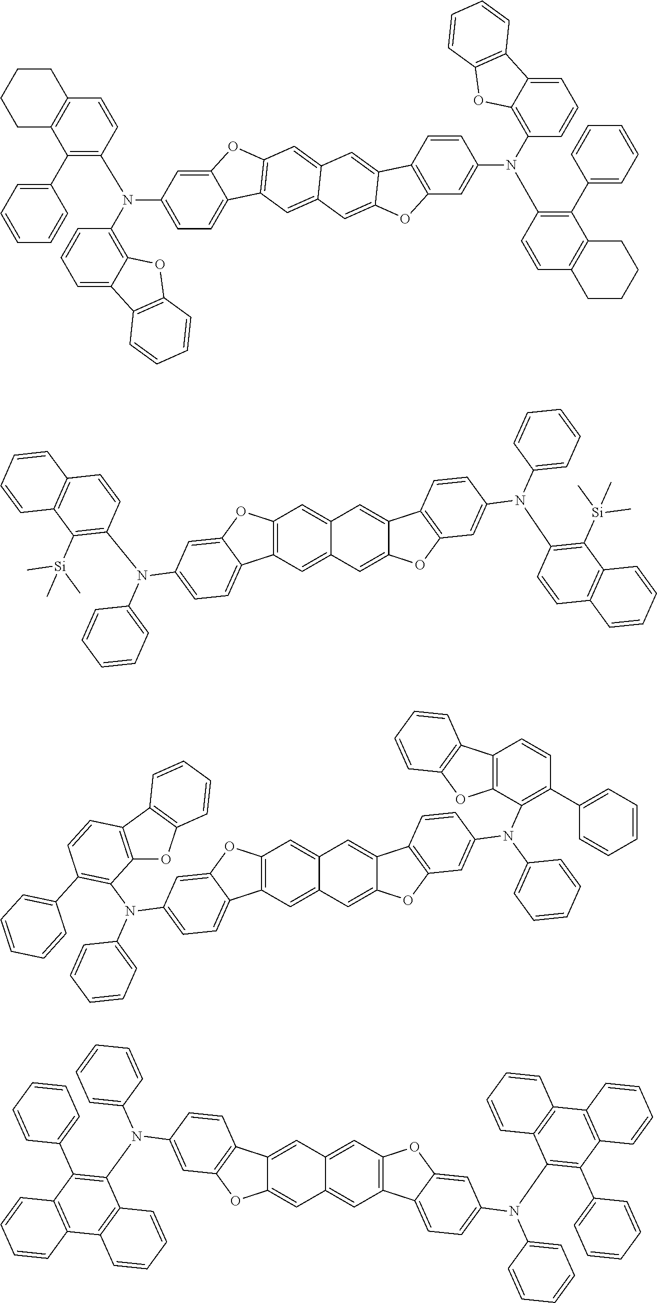

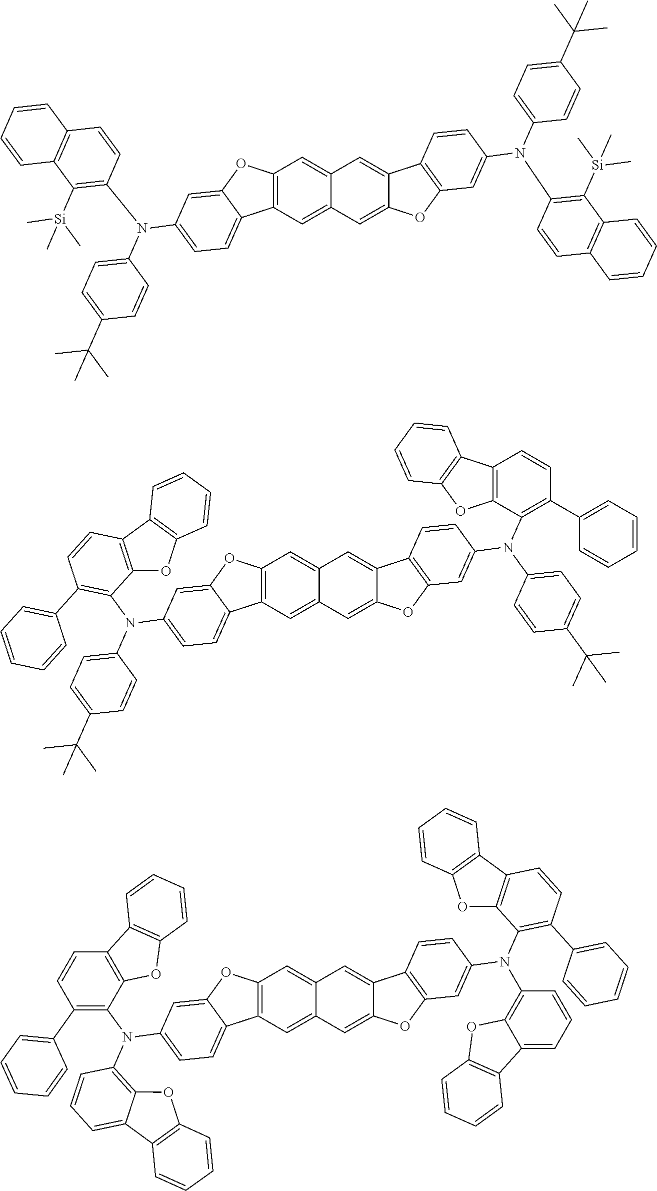 [SMILES: CC(C)(C)c1ccc(N(c2ccc3c(c2)oc2cc4cc5c(cc4cc23)oc2cc(N(c3ccc(C(C)(C)C)cc3)c3ccc4ccccc4c3[Si](C)(C)C)ccc25)c2ccc3ccccc3c2[Si](C)(C)C)cc1 ; CC(C)(C)c1ccc(N(c2ccc3c(c2)oc2cc4cc5c(cc4cc23)oc2cc(N(c3ccc(C(C)(C)C)cc3)c3c(-c4ccccc4)ccc4c3oc3ccccc34)ccc25)c2c(-c3ccccc3)ccc3c2oc2ccccc23)cc1 ; c1ccc(-c2ccc3c(oc4ccccc43)c2N(c2ccc3c(c2)oc2cc4cc5c(cc4cc23)oc2cc(N(c3cccc4c3oc3ccccc34)c3c(-c4ccccc4)ccc4c3oc3ccccc34)ccc25)c2cccc3c2oc2ccccc23)cc1]

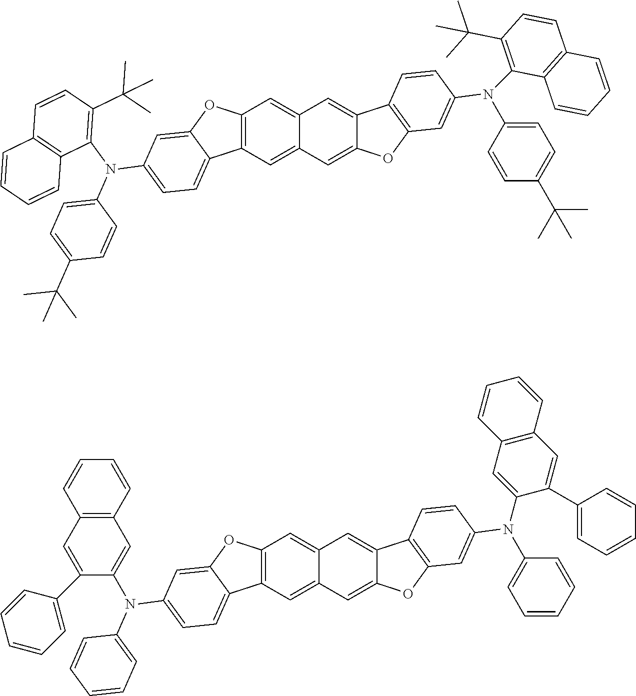 [SMILES: CC(C)(C)c1ccc(N(c2ccc3c(c2)oc2cc4cc5c(cc4cc23)oc2cc(N(c3ccc(C(C)(C)C)cc3)c3c(C(C)(C)C)ccc4ccccc34)ccc25)c2c(C(C)(C)C)ccc3ccccc23)cc1 ; c1ccc(-c2cc3ccccc3cc2N(c2ccccc2)c2ccc3c(c2)oc2cc4cc5c(cc4cc23)oc2cc(N(c3ccccc3)c3cc4ccccc4cc3-c3ccccc3)ccc25)cc1]

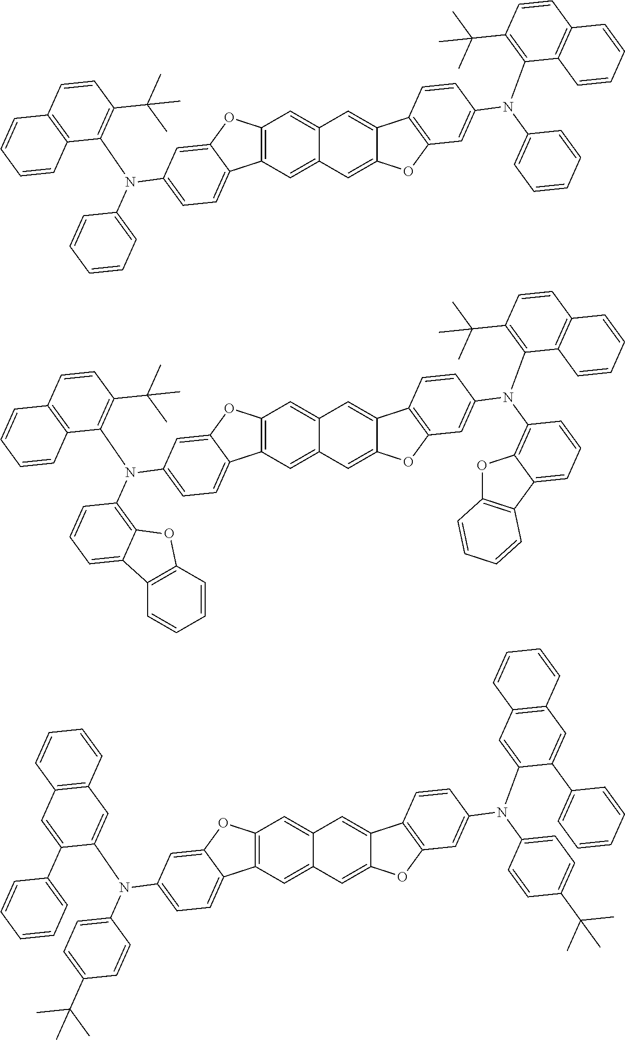 [SMILES: CC(C)(C)c1ccc2ccccc2c1N(c1ccccc1)c1ccc2c(c1)oc1cc3cc4c(cc3cc12)oc1cc(N(c2ccccc2)c2c(C(C)(C)C)ccc3ccccc23)ccc14 ; CC(C)(C)c1ccc2ccccc2c1N(c1ccc2c(c1)oc1cc3cc4c(cc3cc12)oc1cc(N(c2c(C(C)(C)C)ccc3ccccc23)c2cccc3c2oc2ccccc23)ccc14)c1cccc2c1oc1ccccc12 ; CC(C)(C)c1ccc(N(c2ccc3c(c2)oc2cc4cc5c(cc4cc23)oc2cc(N(c3ccc(C(C)(C)C)cc3)c3cc4ccccc4cc3-c3ccccc3)ccc25)c2cc3ccccc3cc2-c2ccccc2)cc1]

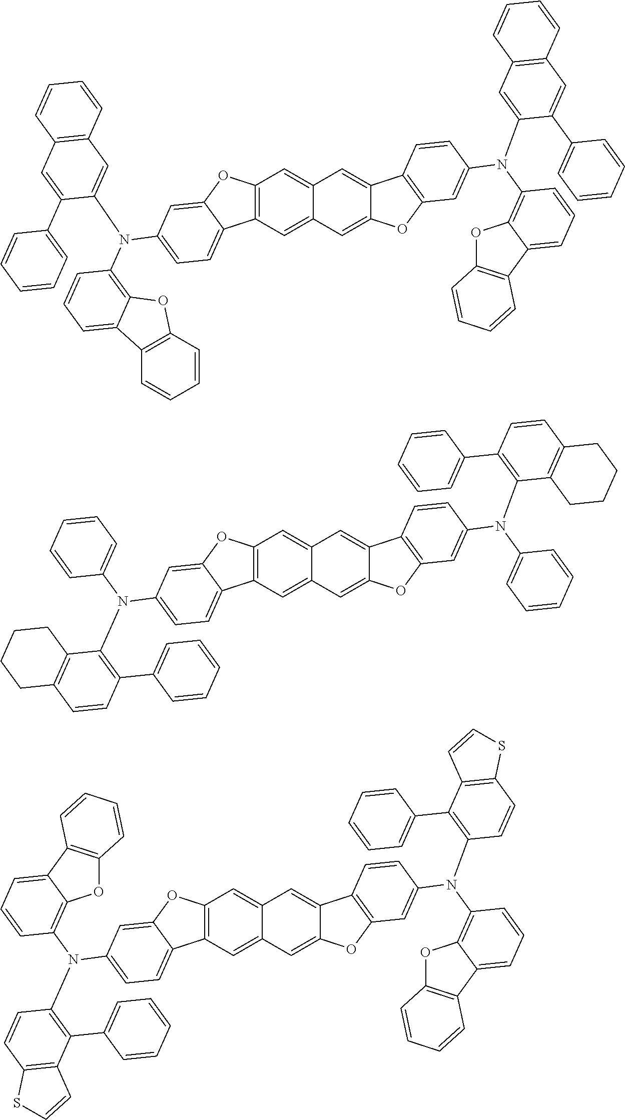 [SMILES: c1ccc(-c2cc3ccccc3cc2N(c2ccc3c(c2)oc2cc4cc5c(cc4cc23)oc2cc(N(c3cc4ccccc4cc3-c3ccccc3)c3cccc4c3oc3ccccc34)ccc25)c2cccc3c2oc2ccccc23)cc1 ; c1ccc(-c2ccc3c(c2N(c2ccccc2)c2ccc4c(c2)oc2cc5cc6c(cc5cc24)oc2cc(N(c4ccccc4)c4c(-c5ccccc5)ccc5c4CCCC5)ccc26)CCCC3)cc1 ; c1ccc(-c2c(N(c3ccc4c(c3)oc3cc5cc6c(cc5cc34)oc3cc(N(c4ccc5sccc5c4-c4ccccc4)c4cccc5c4oc4ccccc45)ccc36)c3cccc4c3oc3ccccc34)ccc3sccc23)cc1]

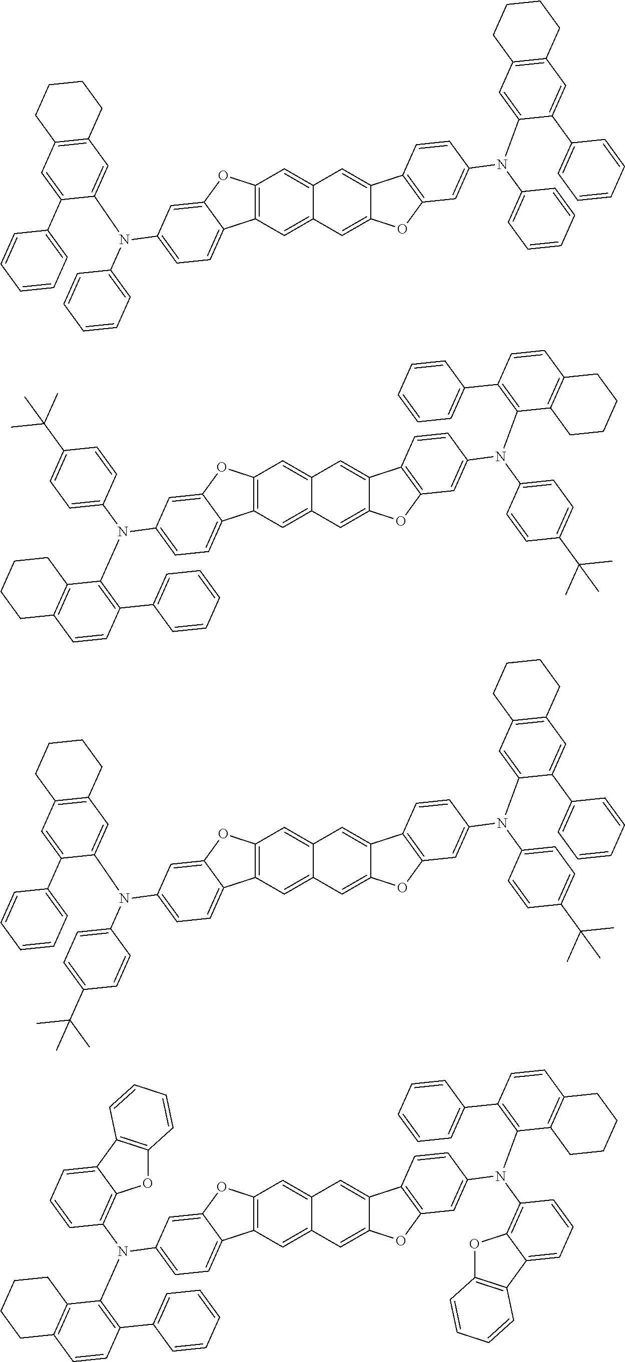

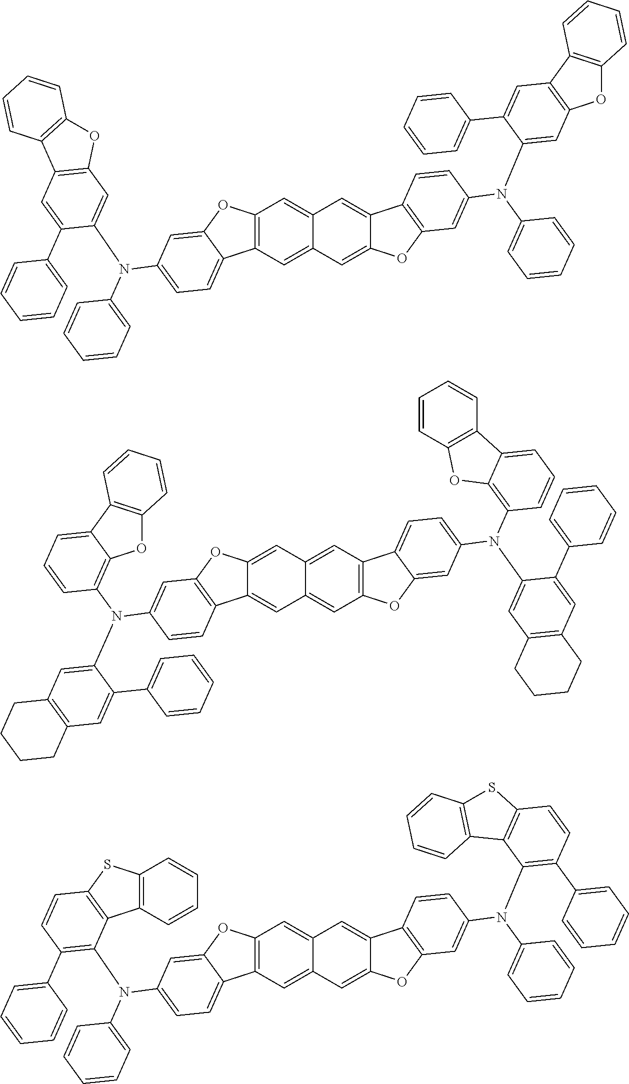 [SMILES: c1ccc(-c2cc3c(cc2N(c2ccccc2)c2ccc4c(c2)oc2cc5cc6c(cc5cc24)oc2cc(N(c4ccccc4)c4cc5oc7ccccc7c5cc4-c4ccccc4)ccc26)oc2ccccc23)cc1 ; c1ccc(-c2cc3c(cc2N(c2ccc4c(c2)oc2cc5cc6c(cc5cc24)oc2cc(N(c4cc5c(cc4-c4ccccc4)CCCC5)c4cccc5c4oc4ccccc45)ccc26)c2cccc4c2oc2ccccc24)CCCC3)cc1 ; c1ccc(-c2ccc3sc4ccccc4c3c2N(c2ccccc2)c2ccc3c(c2)oc2cc4cc5c(cc4cc23)oc2cc(N(c3ccccc3)c3c(-c4ccccc4)ccc4sc6ccccc6c34)ccc25)cc1]

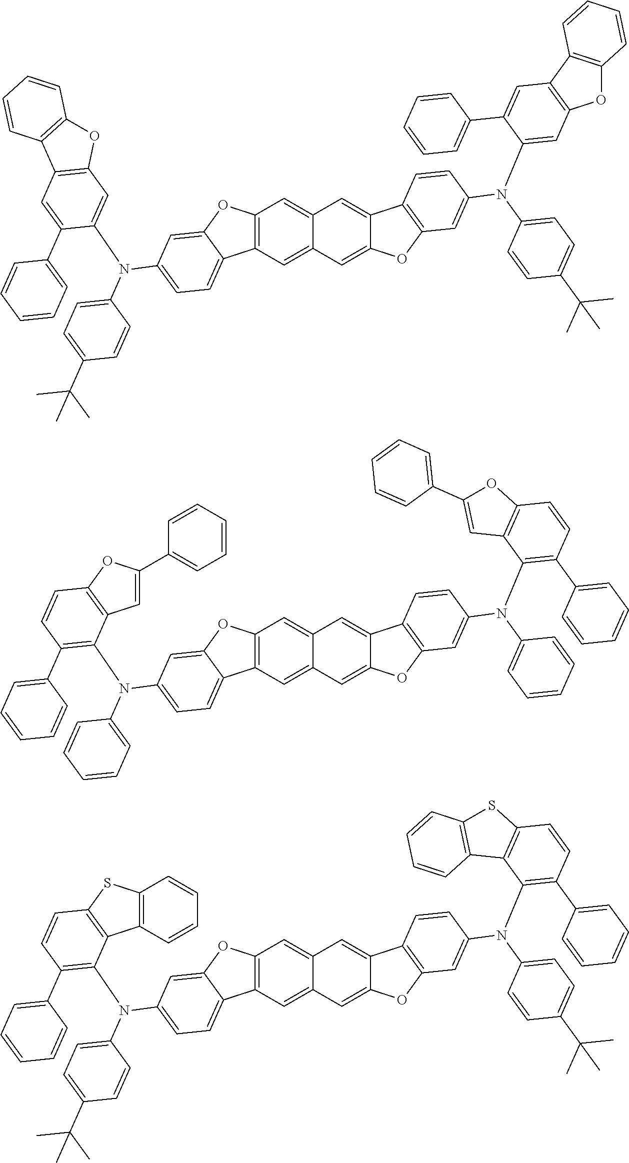 [SMILES: CC(C)(C)c1ccc(N(c2ccc3c(c2)oc2cc4cc5c(cc4cc23)oc2cc(N(c3ccc(C(C)(C)C)cc3)c3cc4oc6ccccc6c4cc3-c3ccccc3)ccc25)c2cc3oc4ccccc4c3cc2-c2ccccc2)cc1 ; c1ccc(-c2cc3c(N(c4ccccc4)c4ccc5c(c4)oc4cc6cc7c(cc6cc45)oc4cc(N(c5ccccc5)c5c(-c6ccccc6)ccc6oc(-c8ccccc8)cc56)ccc47)c(-c4ccccc4)ccc3o2)cc1 ; CC(C)(C)c1ccc(N(c2ccc3c(c2)oc2cc4cc5c(cc4cc23)oc2cc(N(c3ccc(C(C)(C)C)cc3)c3c(-c4ccccc4)ccc4sc6ccccc6c34)ccc25)c2c(-c3ccccc3)ccc3sc4ccccc4c23)cc1]

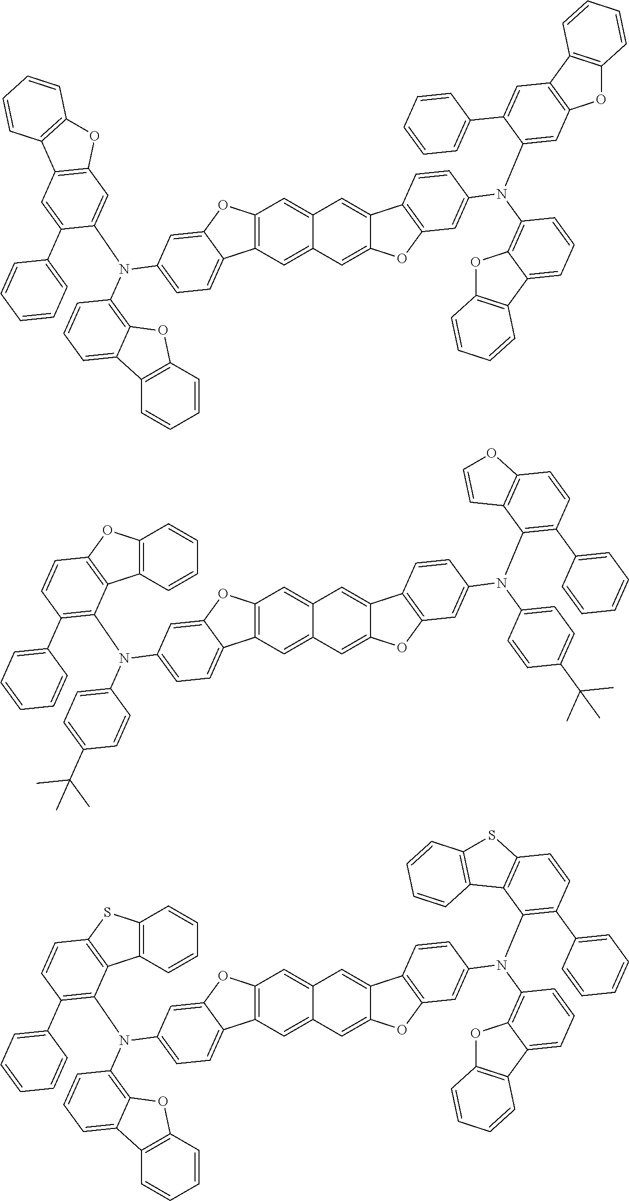 [SMILES: c1ccc(-c2cc3c(cc2N(c2ccc4c(c2)oc2cc5cc6c(cc5cc24)oc2cc(N(c4cc5oc7ccccc7c5cc4-c4ccccc4)c4cccc5c4oc4ccccc45)ccc26)c2cccc4c2oc2ccccc24)oc2ccccc23)cc1 ; CC(C)(C)c1ccc(N(c2ccc3c(c2)oc2cc4cc5c(cc4cc23)oc2cc(N(c3ccc(C(C)(C)C)cc3)c3c(-c4ccccc4)ccc4oc6ccccc6c34)ccc25)c2c(-c3ccccc3)ccc3occc23)cc1 ; c1ccc(-c2ccc3sc4ccccc4c3c2N(c2ccc3c(c2)oc2cc4cc5c(cc4cc23)oc2cc(N(c3cccc4c3oc3ccccc34)c3c(-c4ccccc4)ccc4sc6ccccc6c34)ccc25)c2cccc3c2oc2ccccc23)cc1]

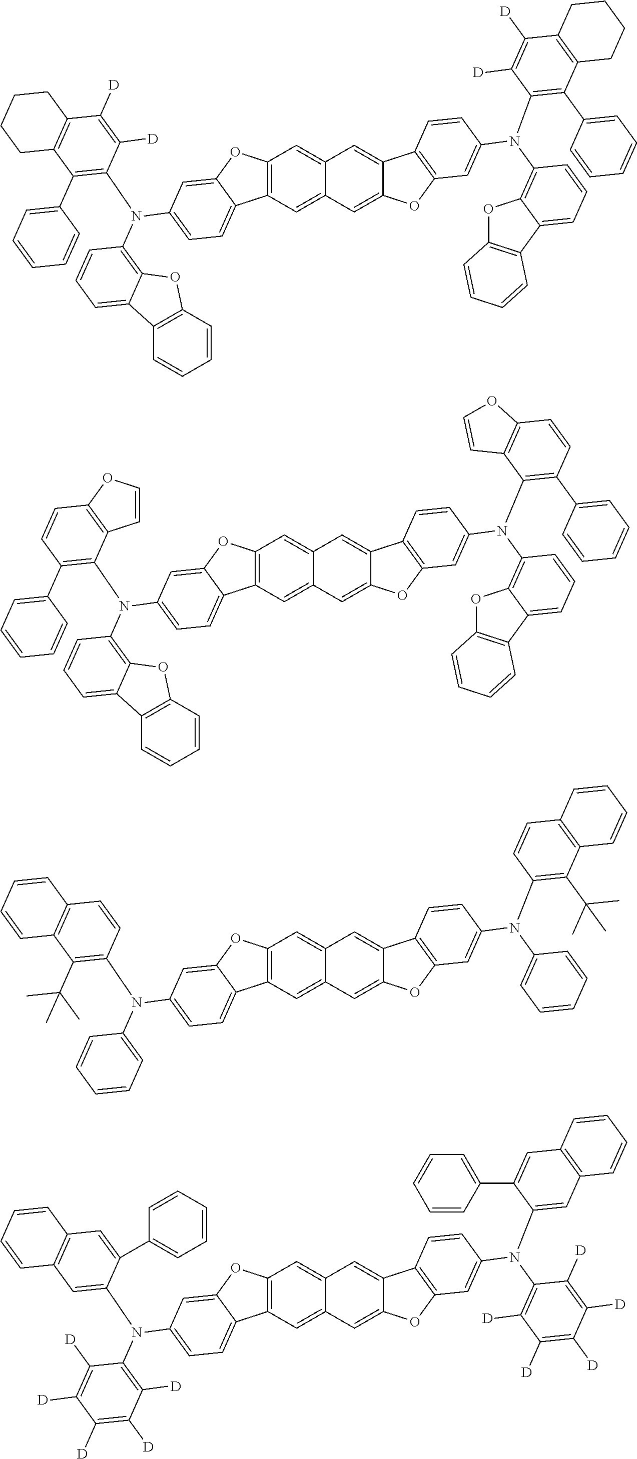

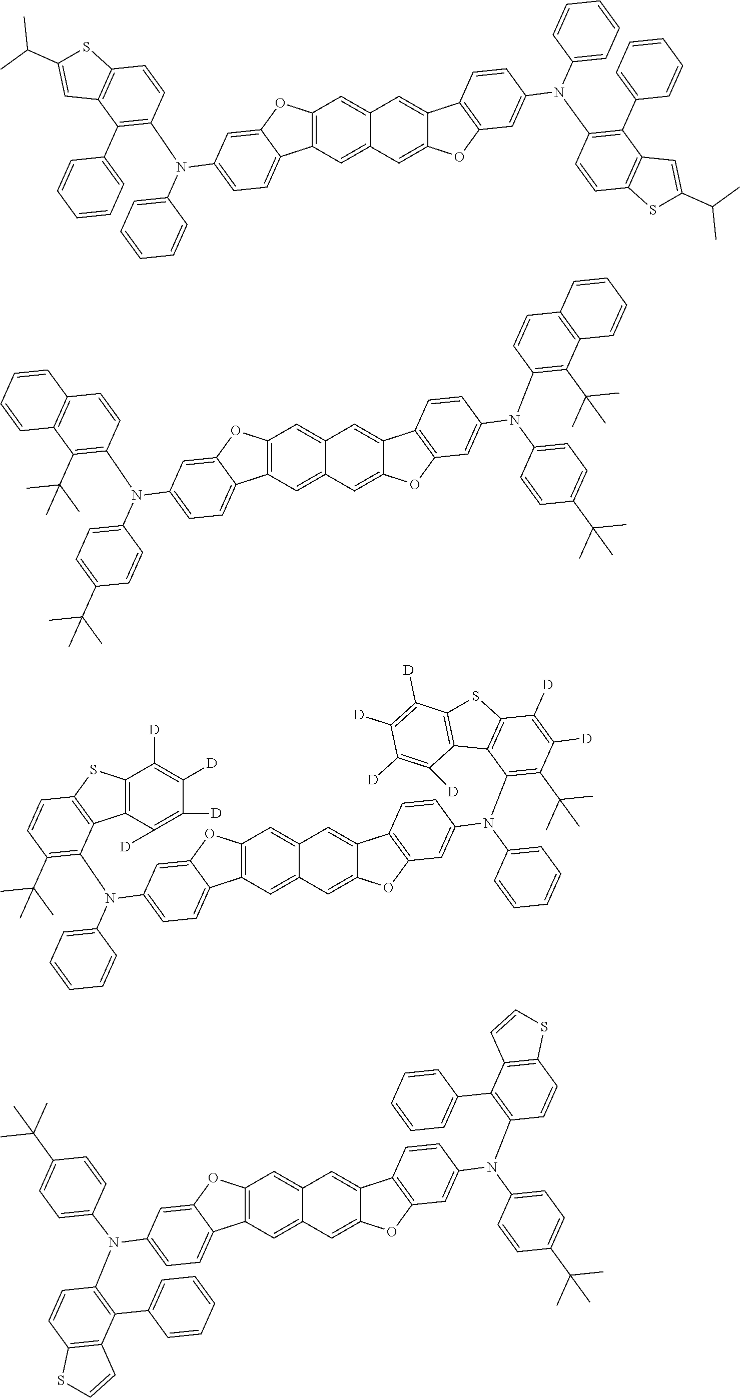

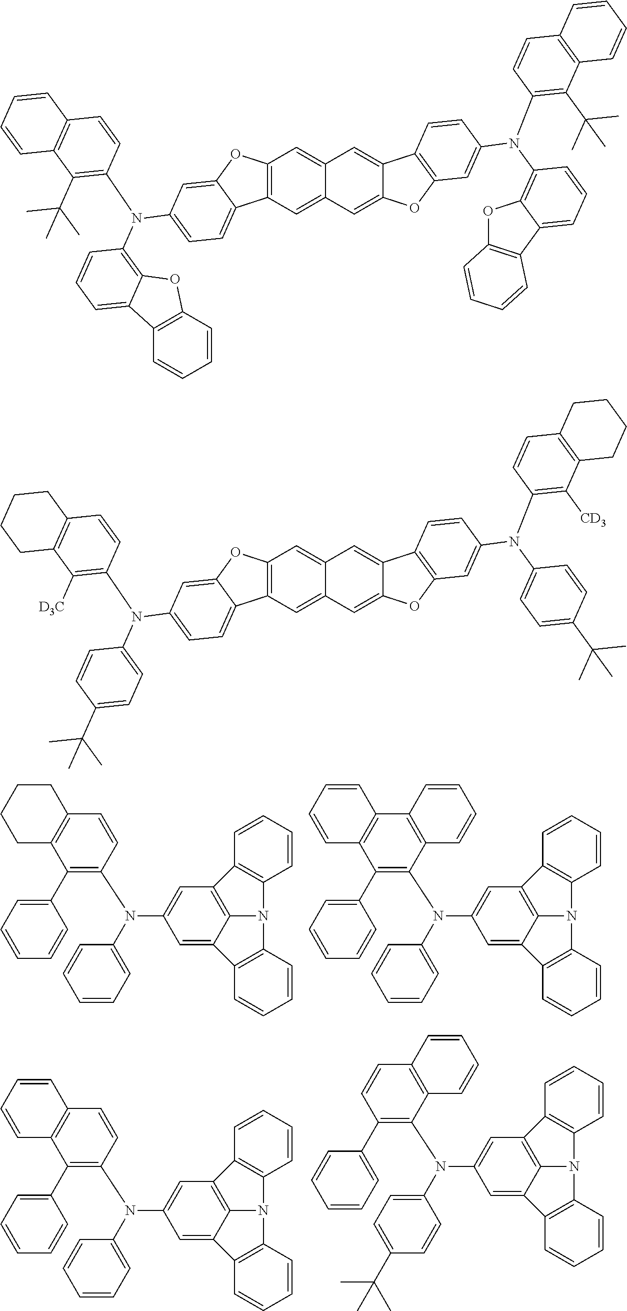

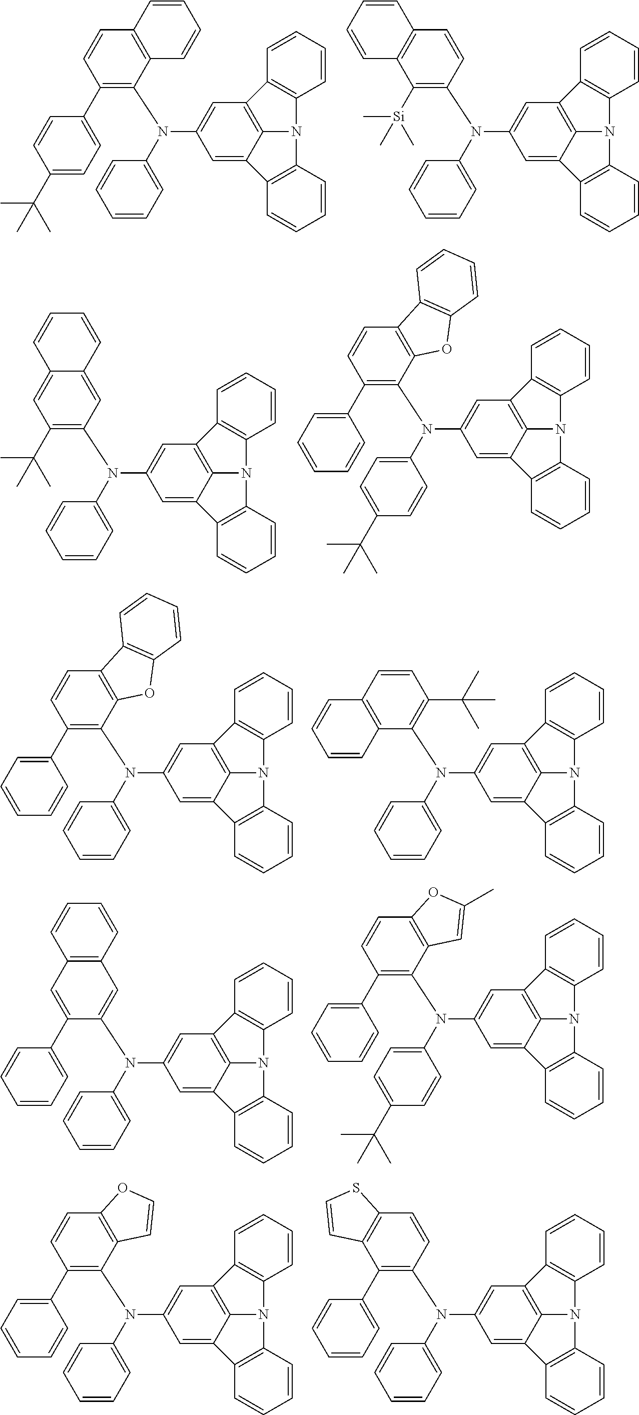

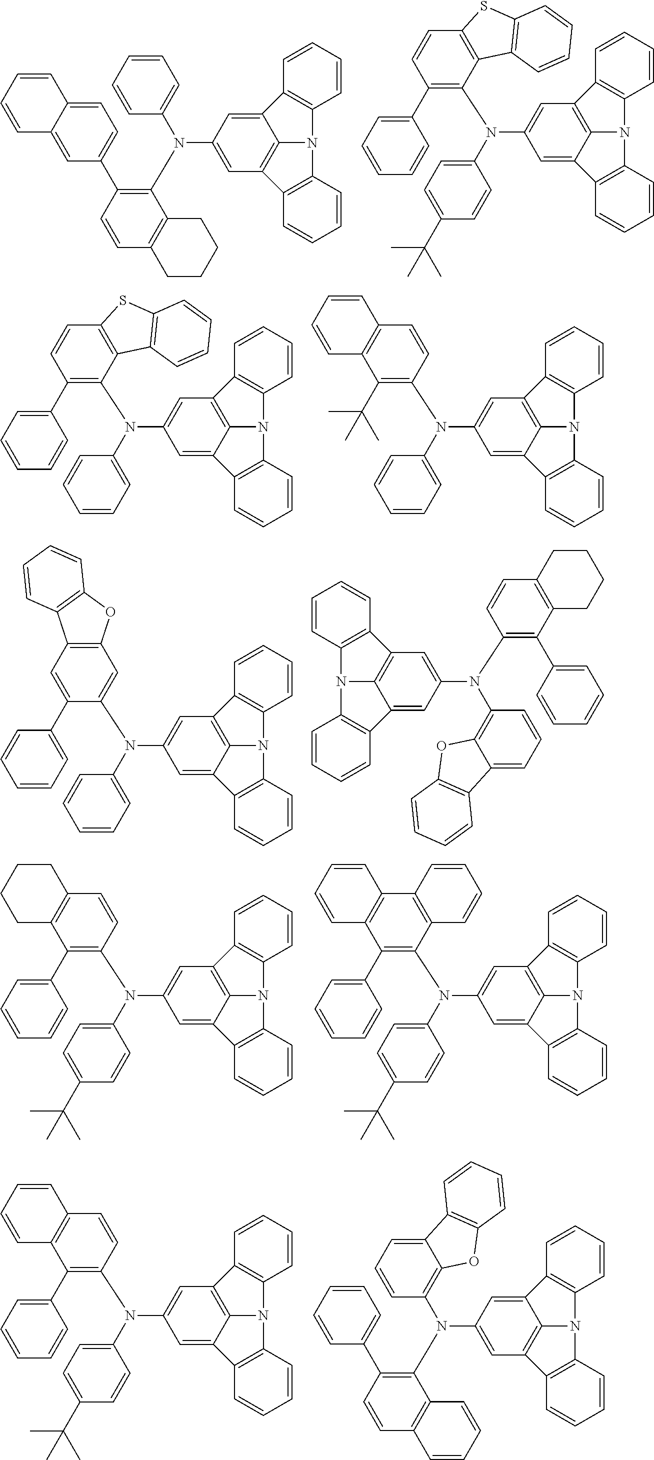

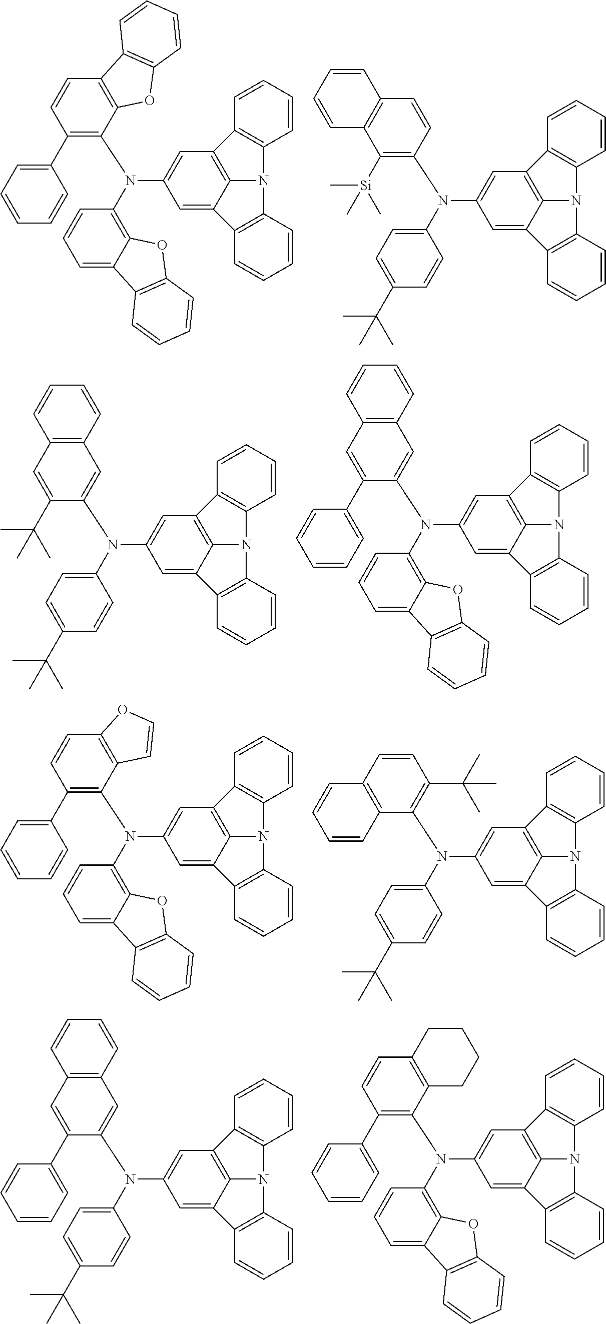

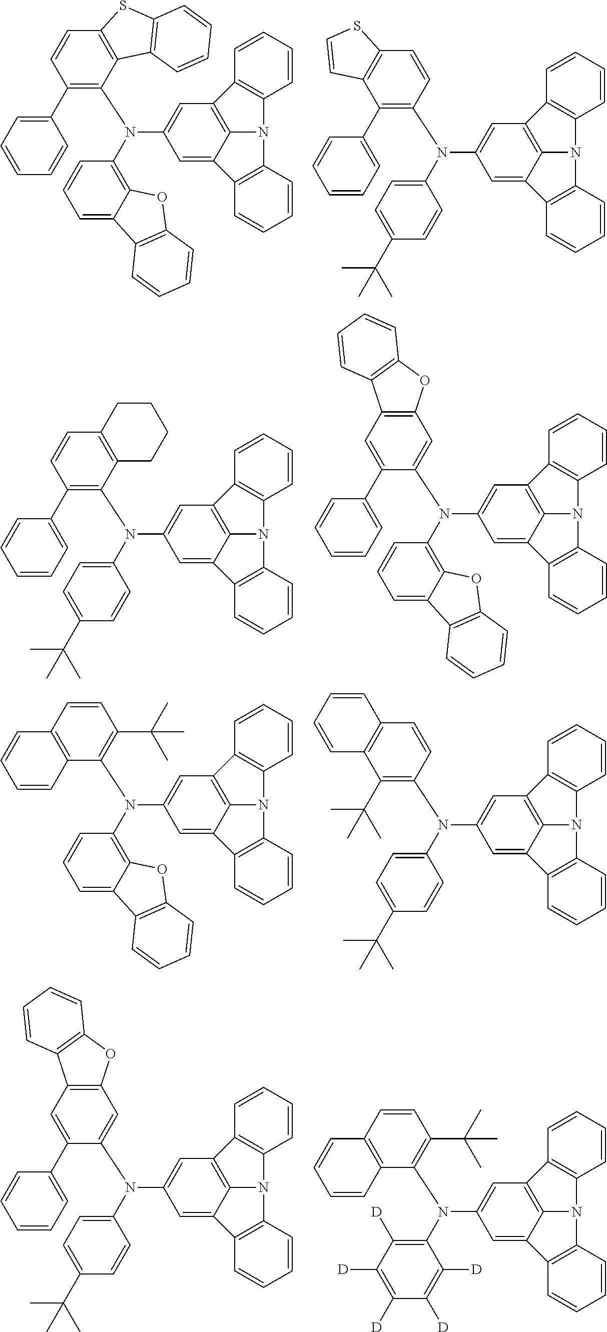

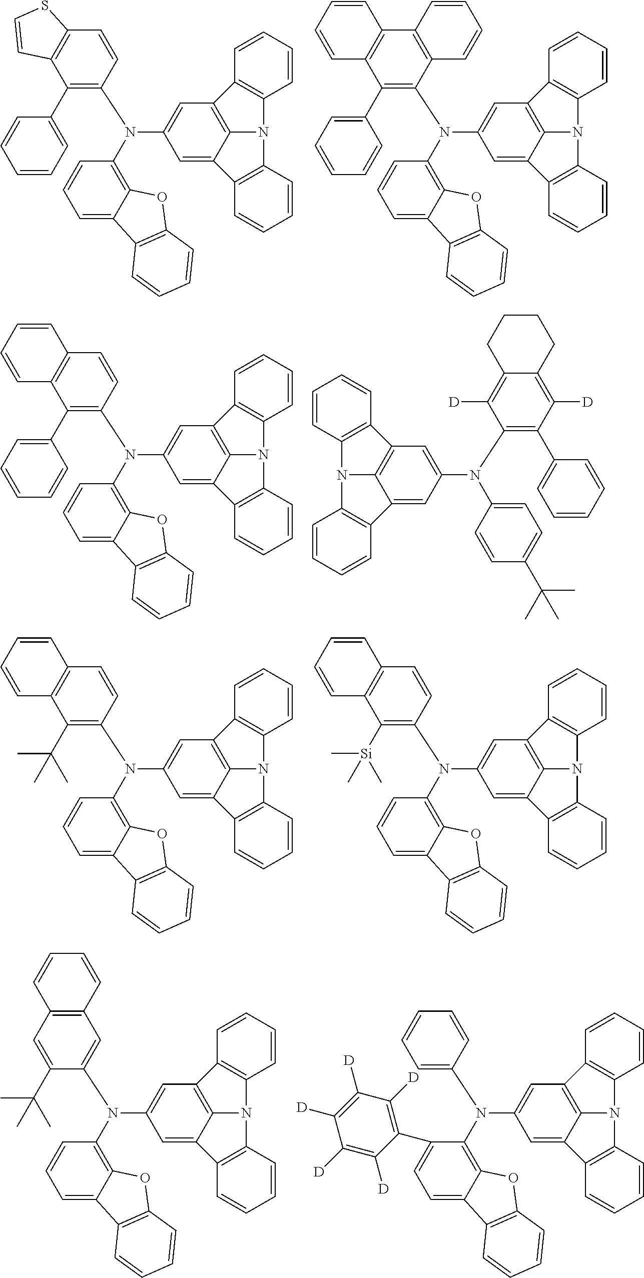

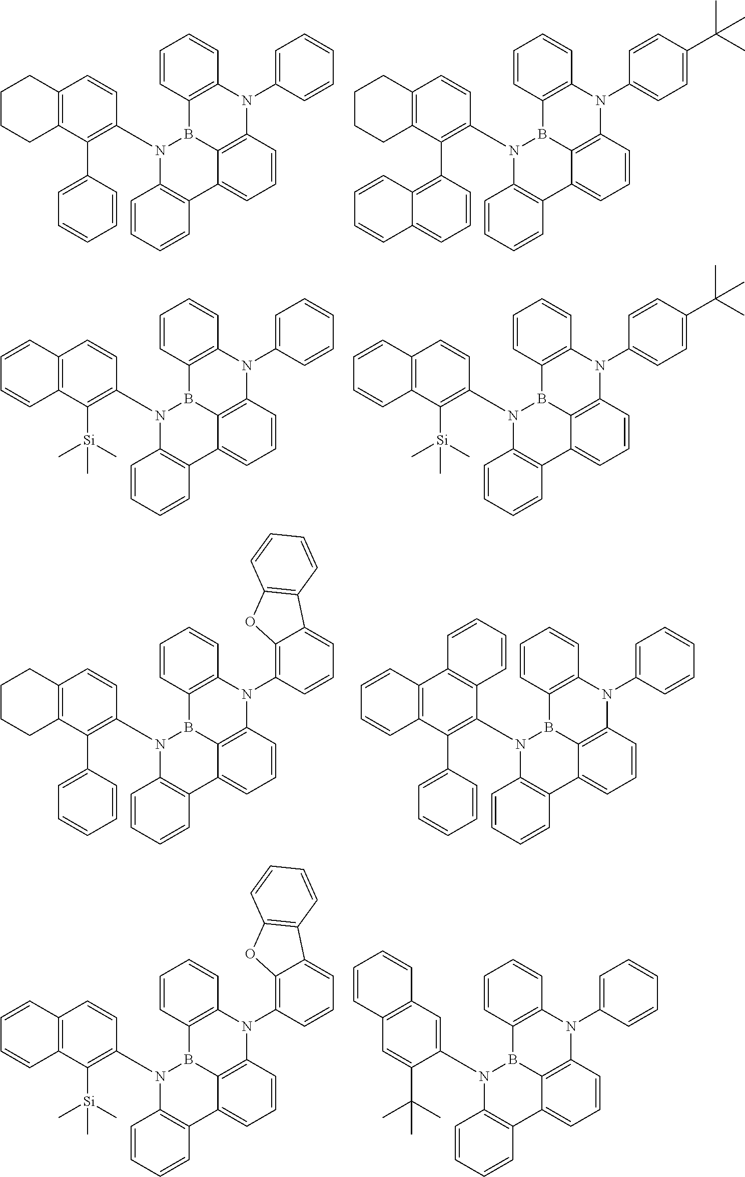

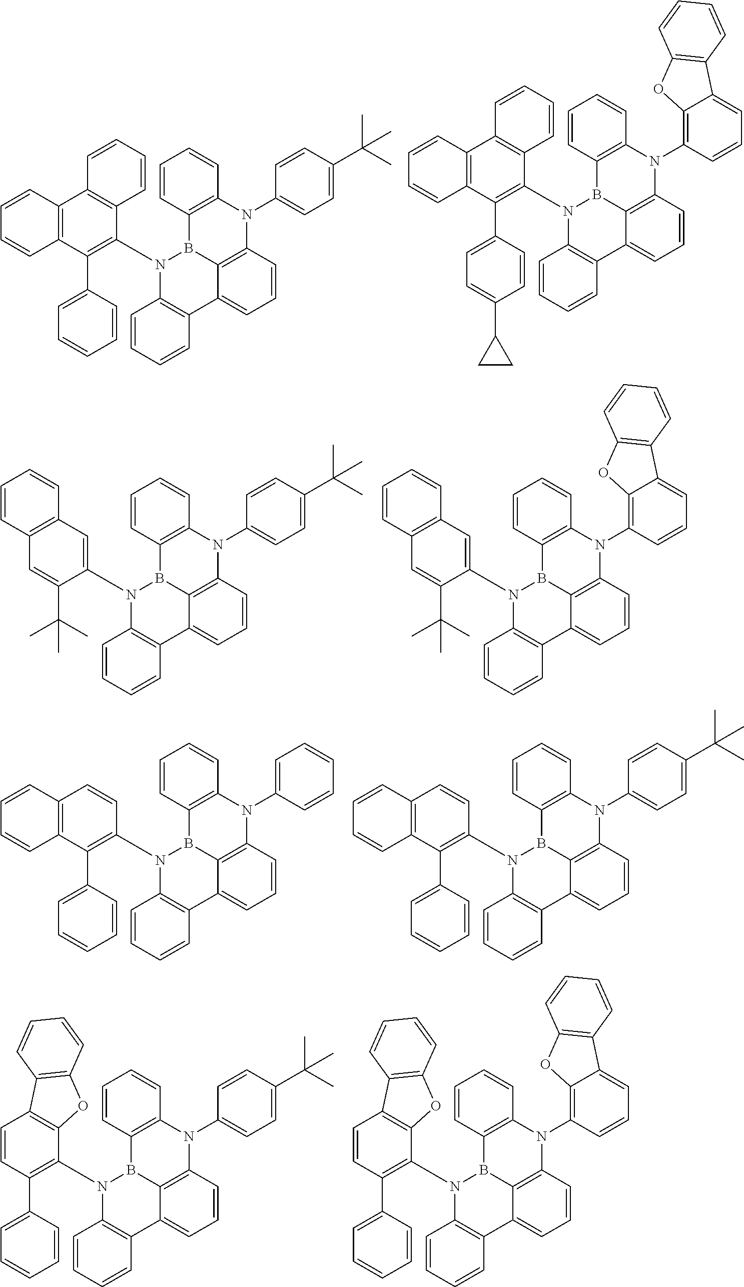

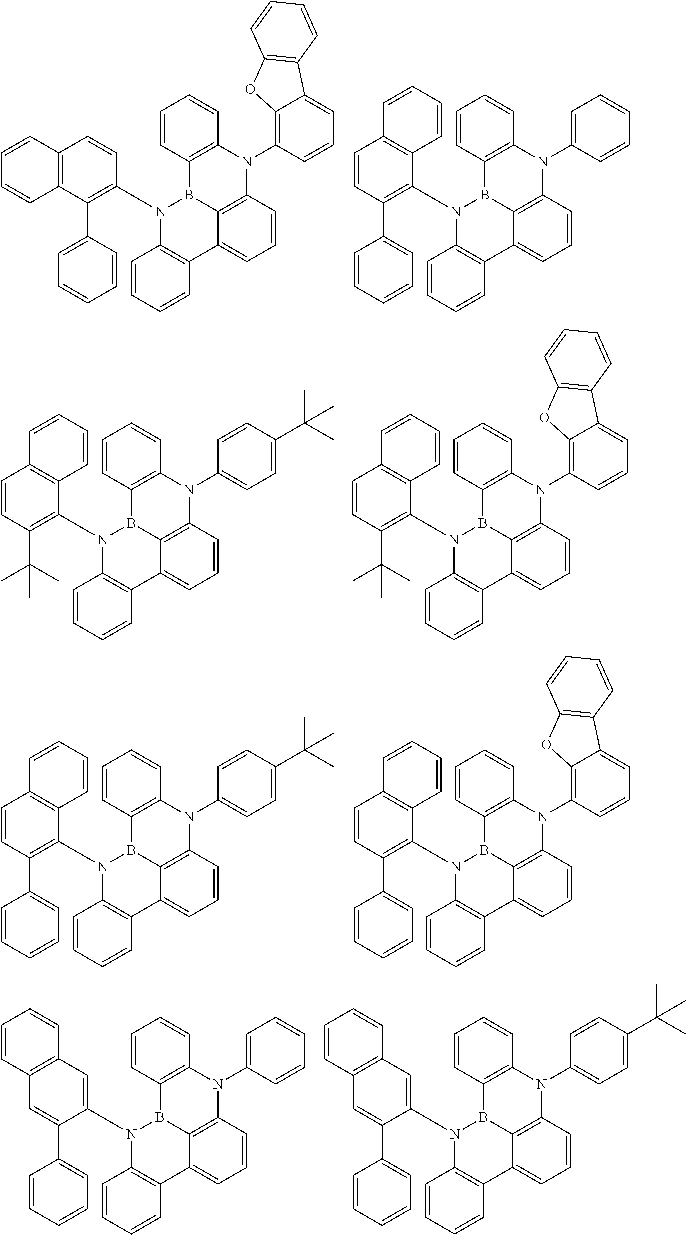

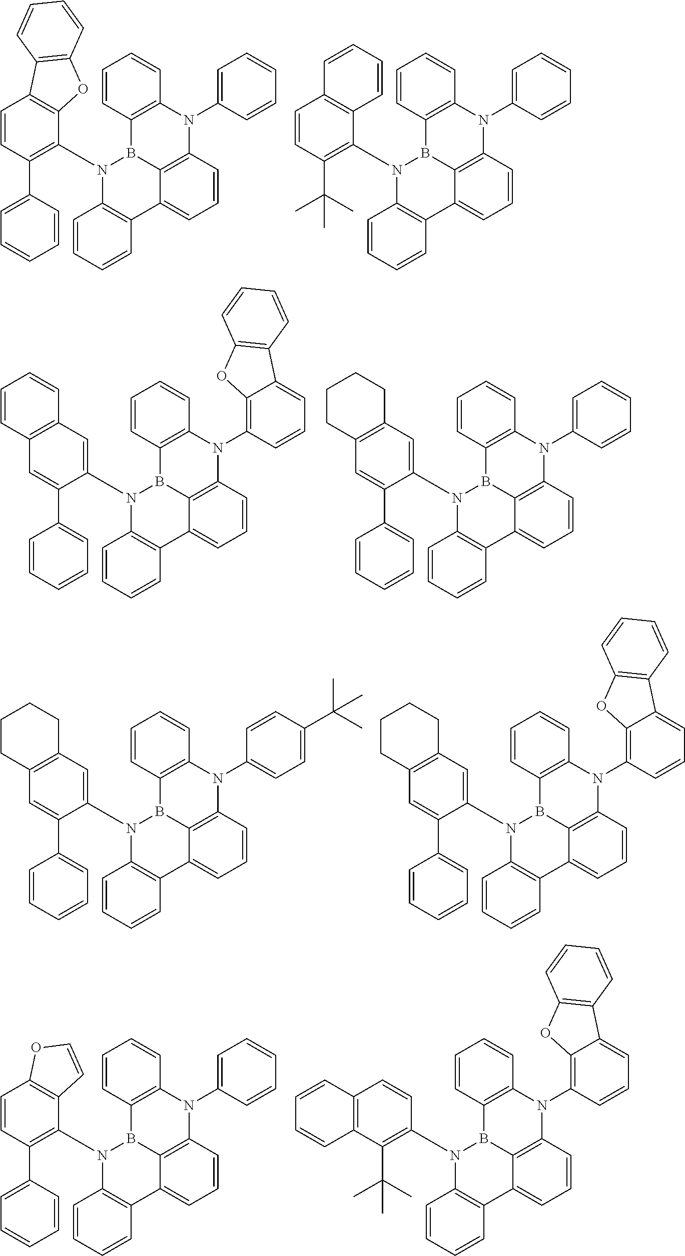

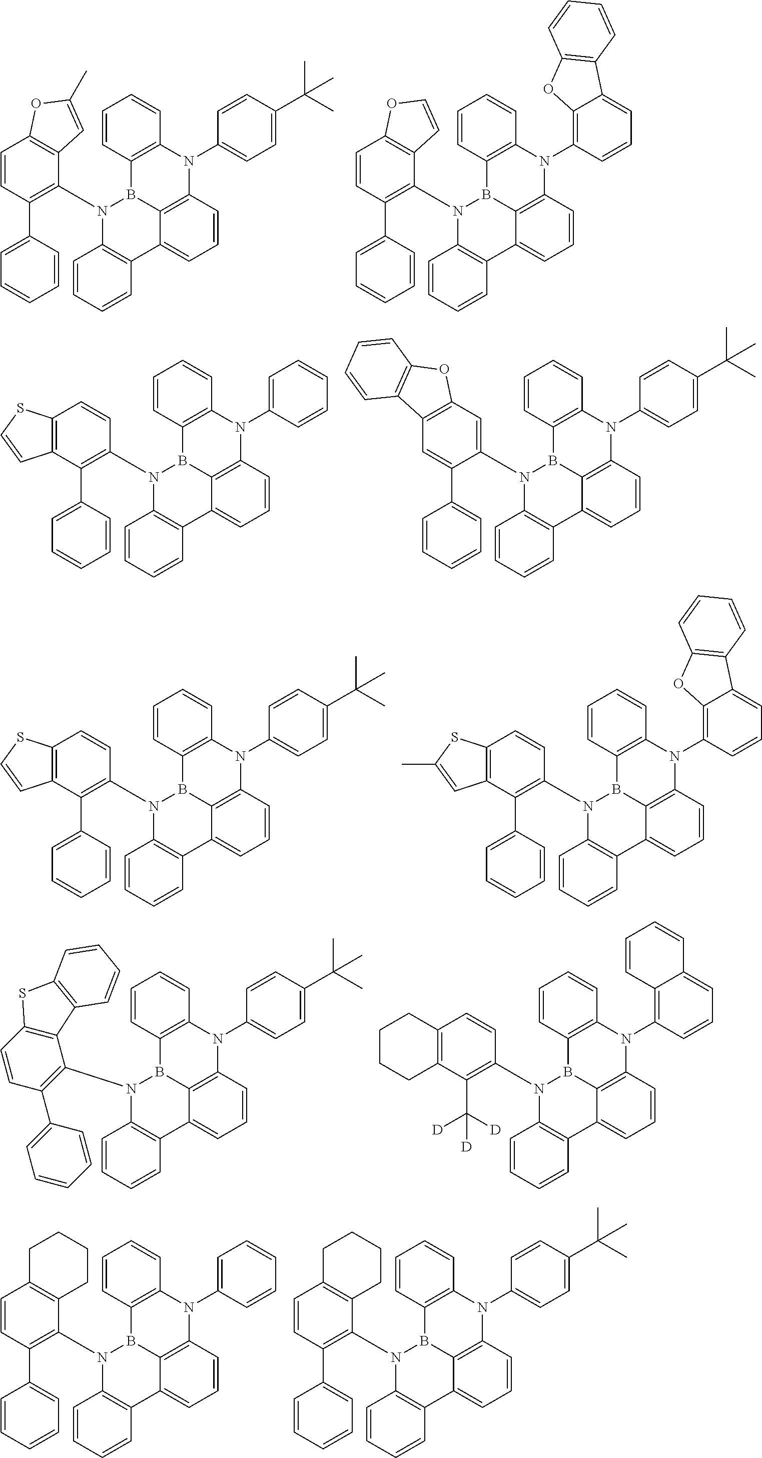

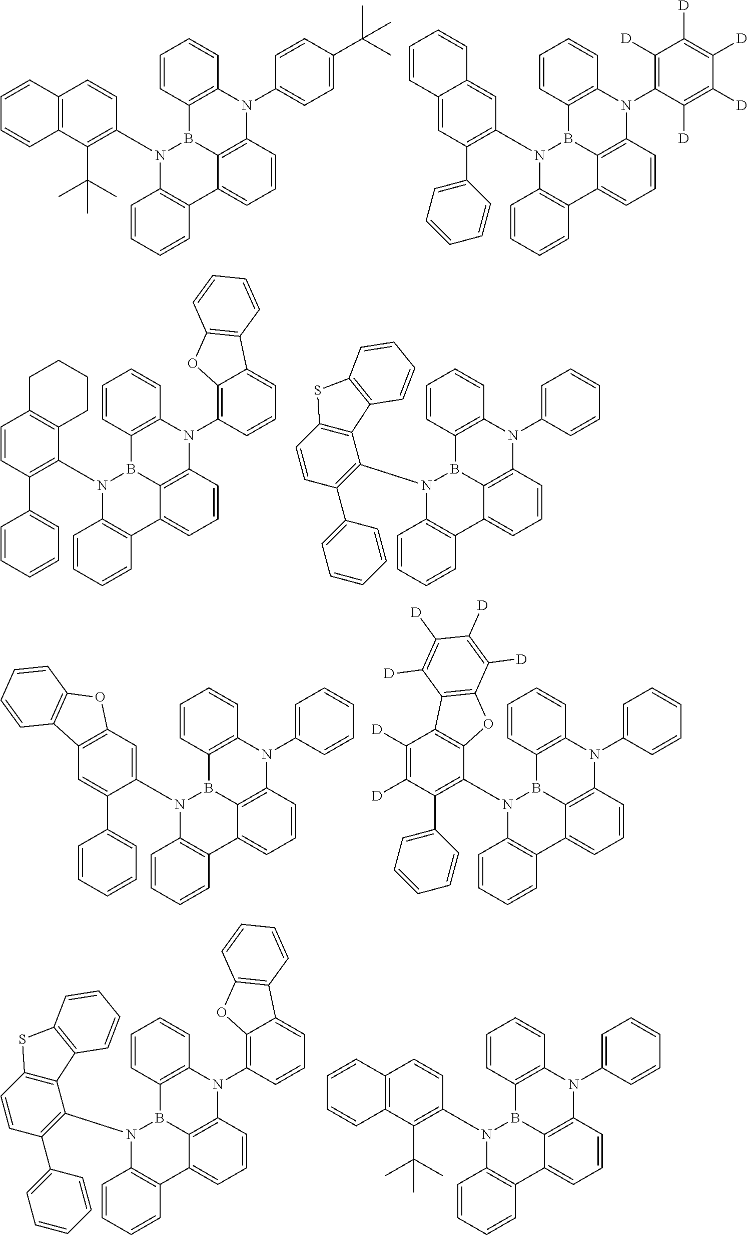

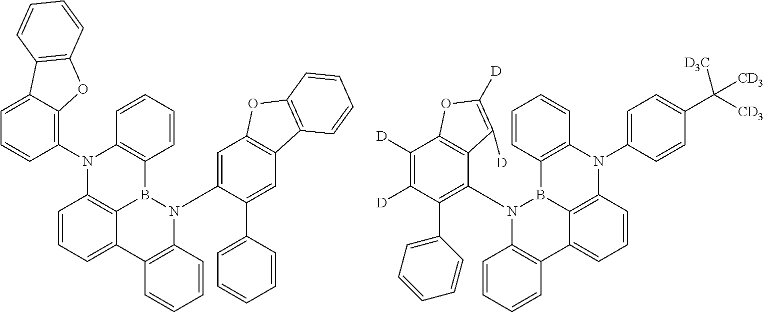

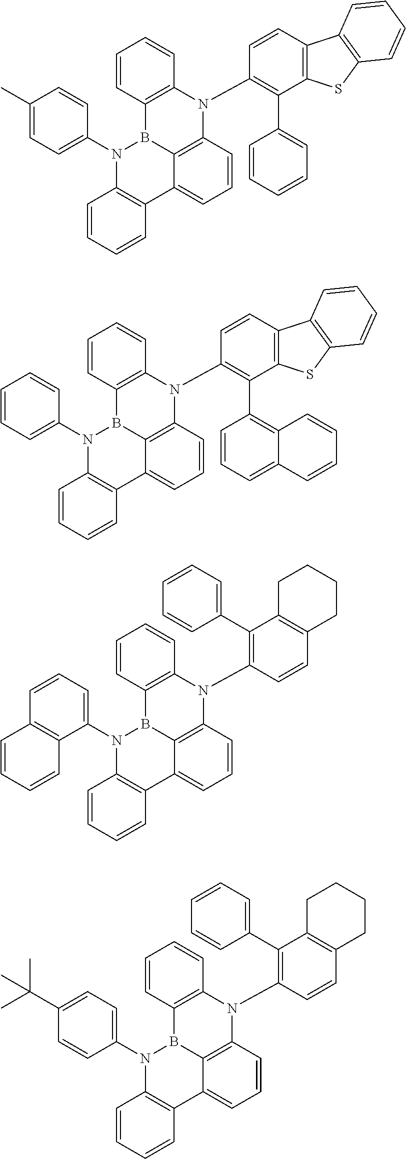 [SMILES: Cc1ccc(N2B3c4ccccc4N(c4ccc5c(sc6ccccc65)c4-c4ccccc4)c4cccc(c43)-c3ccccc32)cc1 ; c1ccc(N2B3c4ccccc4N(c4ccc5c(sc6ccccc65)c4-c4cccc5ccccc45)c4cccc(c43)-c3ccccc32)cc1 ; c1ccc(-c2c(N3c4ccccc4B4c5c(cccc53)-c3ccccc3N4c3cccc4ccccc34)ccc3c2CCCC3)cc1 ; CC(C)(C)c1ccc(N2B3c4ccccc4N(c4ccc5c(c4-c4ccccc4)CCCC5)c4cccc(c43)-c3ccccc32)cc1]

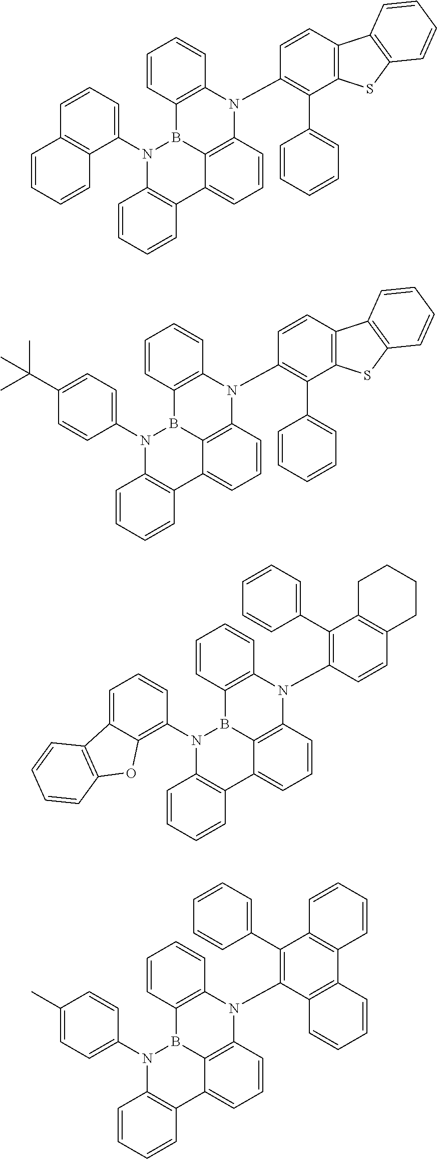 [SMILES: c1ccc(-c2c(N3c4ccccc4B4c5c(cccc53)-c3ccccc3N4c3cccc4ccccc34)ccc3c2sc2ccccc23)cc1 ; CC(C)(C)c1ccc(N2B3c4ccccc4N(c4ccc5c(sc6ccccc65)c4-c4ccccc4)c4cccc(c43)-c3ccccc32)cc1 ; c1ccc(-c2c(N3c4ccccc4B4c5c(cccc53)-c3ccccc3N4c3cccc4c3oc3ccccc34)ccc3c2CCCC3)cc1 ; Cc1ccc(N2B3c4ccccc4N(c4c(-c5ccccc5)c5ccccc5c5ccccc45)c4cccc(c43)-c3ccccc32)cc1]

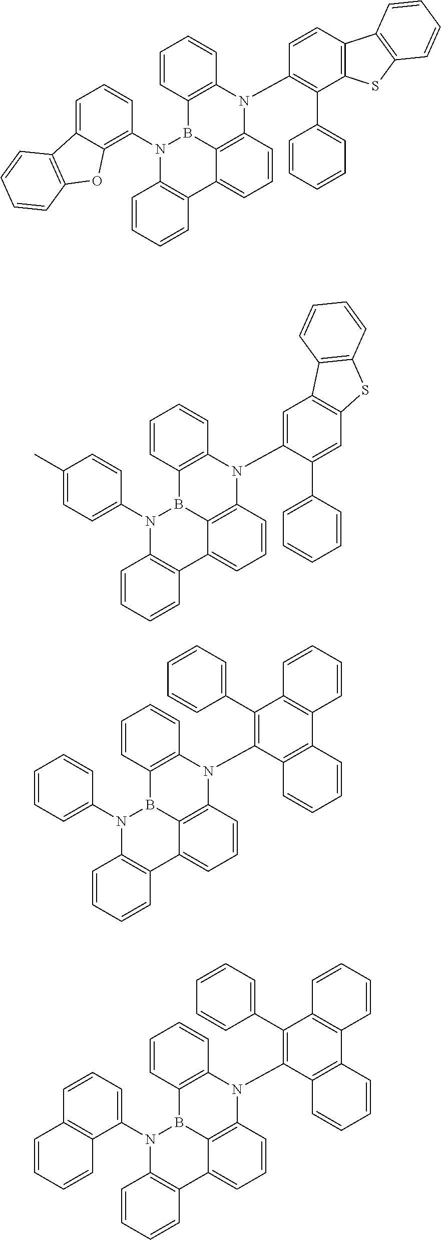

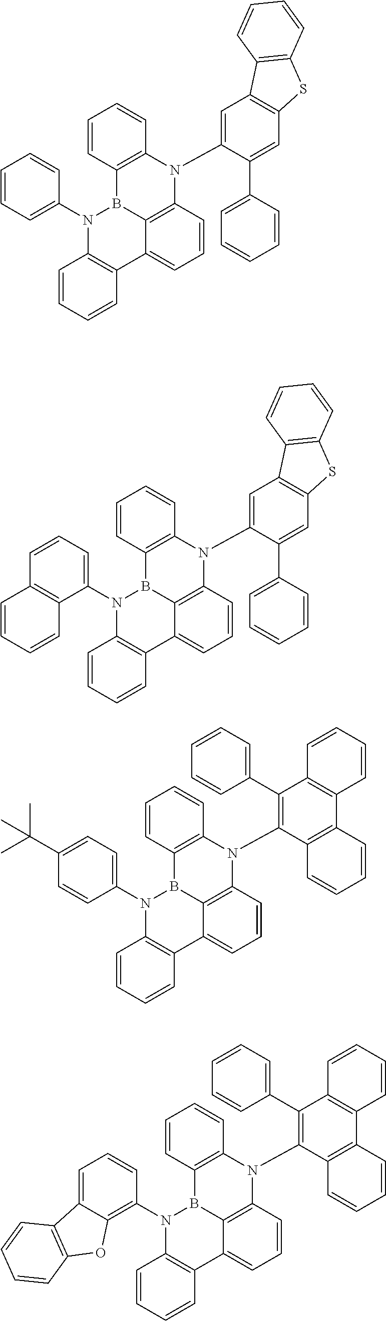 [SMILES: c1ccc(-c2cc3sc4ccccc4c3cc2N2c3ccccc3B3c4c(cccc42)-c2ccccc2N3c2ccccc2)cc1 ; c1ccc(-c2cc3sc4ccccc4c3cc2N2c3ccccc3B3c4c(cccc42)-c2ccccc2N3c2cccc3ccccc23)cc1 ; CC(C)(C)c1ccc(N2B3c4ccccc4N(c4c(-c5ccccc5)c5ccccc5c5ccccc45)c4cccc(c43)-c3ccccc32)cc1 ; c1ccc(-c2c(N3c4ccccc4B4c5c(cccc53)-c3ccccc3N4c3cccc4c3oc3ccccc34)c3ccccc3c3ccccc23)cc1]

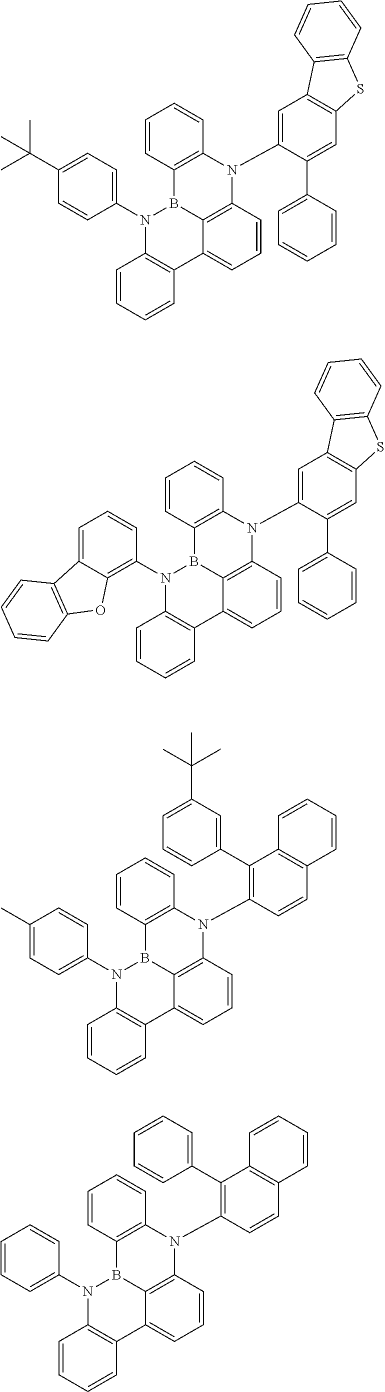 [SMILES: CC(C)(C)c1ccc(N2B3c4ccccc4N(c4cc5c(cc4-c4ccccc4)sc4ccccc45)c4cccc(c43)-c3ccccc32)cc1 ; c1ccc(-c2cc3sc4ccccc4c3cc2N2c3ccccc3B3c4c(cccc42)-c2ccccc2N3c2cccc3c2oc2ccccc23)cc1 ; Cc1ccc(N2B3c4ccccc4N(c4ccc5ccccc5c4-c4cccc(C(C)(C)C)c4)c4cccc(c43)-c3ccccc32)cc1 ; c1ccc(-c2c(N3c4ccccc4B4c5c(cccc53)-c3ccccc3N4c3ccccc3)ccc3ccccc23)cc1]

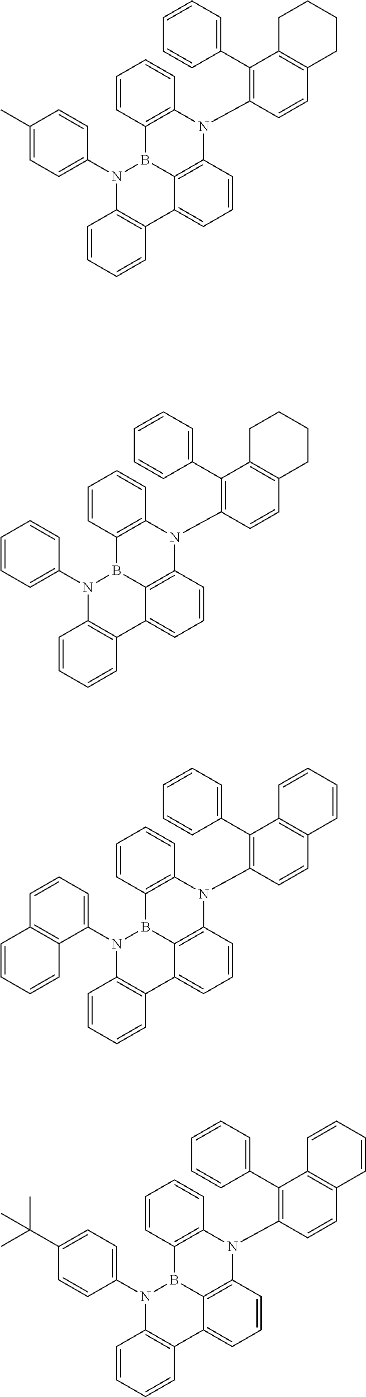 [SMILES: Cc1ccc(N2B3c4ccccc4N(c4ccc5c(c4-c4ccccc4)CCCC5)c4cccc(c43)-c3ccccc32)cc1 ; c1ccc(-c2c(N3c4ccccc4B4c5c(cccc53)-c3ccccc3N4c3ccccc3)ccc3c2CCCC3)cc1 ; c1ccc(-c2c(N3c4ccccc4B4c5c(cccc53)-c3ccccc3N4c3cccc4ccccc34)ccc3ccccc23)cc1 ; CC(C)(C)c1ccc(N2B3c4ccccc4N(c4ccc5ccccc5c4-c4ccccc4)c4cccc(c43)-c3ccccc32)cc1]

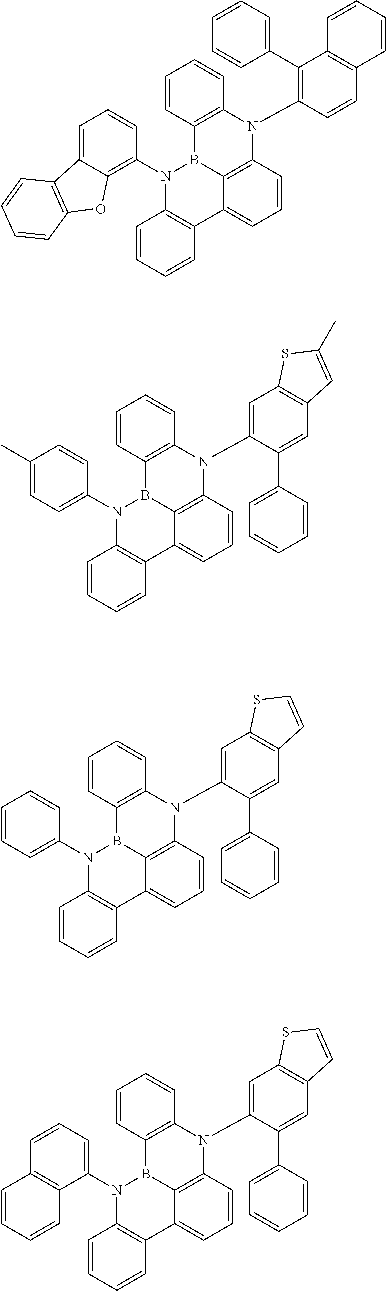

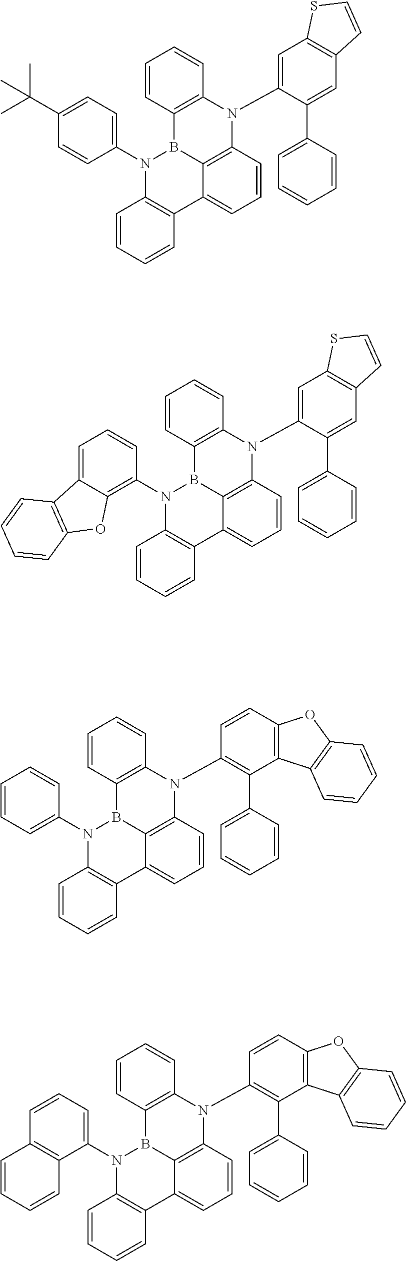 [SMILES: CC(C)(C)c1ccc(N2B3c4ccccc4N(c4cc5sccc5cc4-c4ccccc4)c4cccc(c43)-c3ccccc32)cc1 ; c1ccc(-c2cc3ccsc3cc2N2c3ccccc3B3c4c(cccc42)-c2ccccc2N3c2cccc3c2oc2ccccc23)cc1 ; c1ccc(-c2c(N3c4ccccc4B4c5c(cccc53)-c3ccccc3N4c3ccccc3)ccc3oc4ccccc4c23)cc1 ; c1ccc(-c2c(N3c4ccccc4B4c5c(cccc53)-c3ccccc3N4c3cccc4ccccc34)ccc3oc4ccccc4c23)cc1]

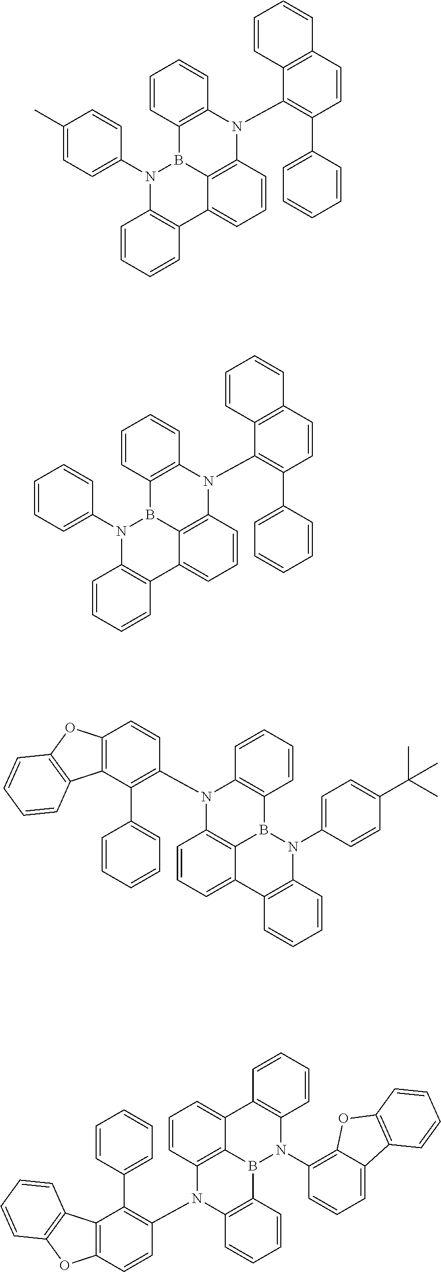 [SMILES: Cc1ccc(N2B3c4ccccc4N(c4c(-c5ccccc5)ccc5ccccc45)c4cccc(c43)-c3ccccc32)cc1 ; c1ccc(-c2ccc3ccccc3c2N2c3ccccc3B3c4c(cccc42)-c2ccccc2N3c2ccccc2)cc1 ; CC(C)(C)c1ccc(N2B3c4ccccc4N(c4ccc5oc6ccccc6c5c4-c4ccccc4)c4cccc(c43)-c3ccccc32)cc1 ; c1ccc(-c2c(N3c4ccccc4B4c5c(cccc53)-c3ccccc3N4c3cccc4c3oc3ccccc34)ccc3oc4ccccc4c23)cc1]

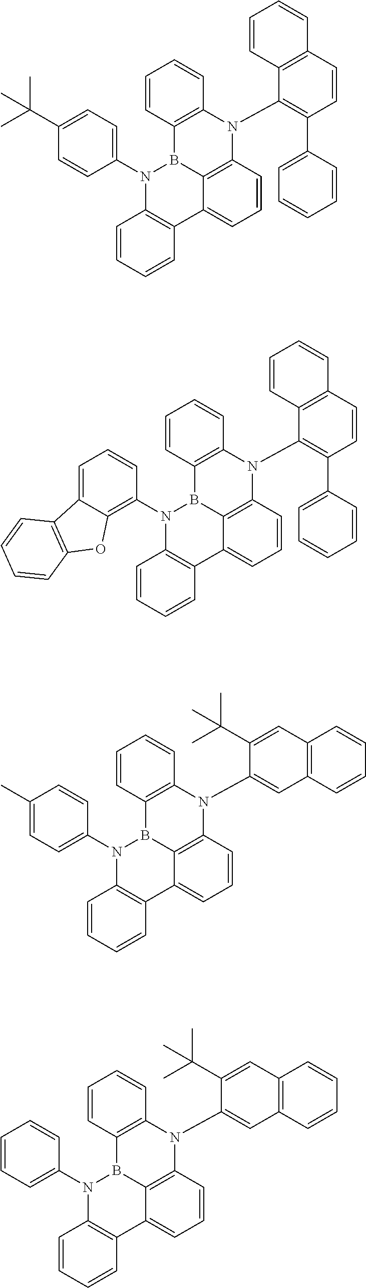 [SMILES: CC(C)(C)c1ccc(N2B3c4ccccc4N(c4c(-c5ccccc5)ccc5ccccc45)c4cccc(c43)-c3ccccc32)cc1 ; c1ccc(-c2ccc3ccccc3c2N2c3ccccc3B3c4c(cccc42)-c2ccccc2N3c2cccc3c2oc2ccccc23)cc1 ; Cc1ccc(N2B3c4ccccc4N(c4cc5ccccc5cc4C(C)(C)C)c4cccc(c43)-c3ccccc32)cc1 ; CC(C)(C)c1cc2ccccc2cc1N1c2ccccc2B2c3c(cccc31)-c1ccccc1N2c1ccccc1]

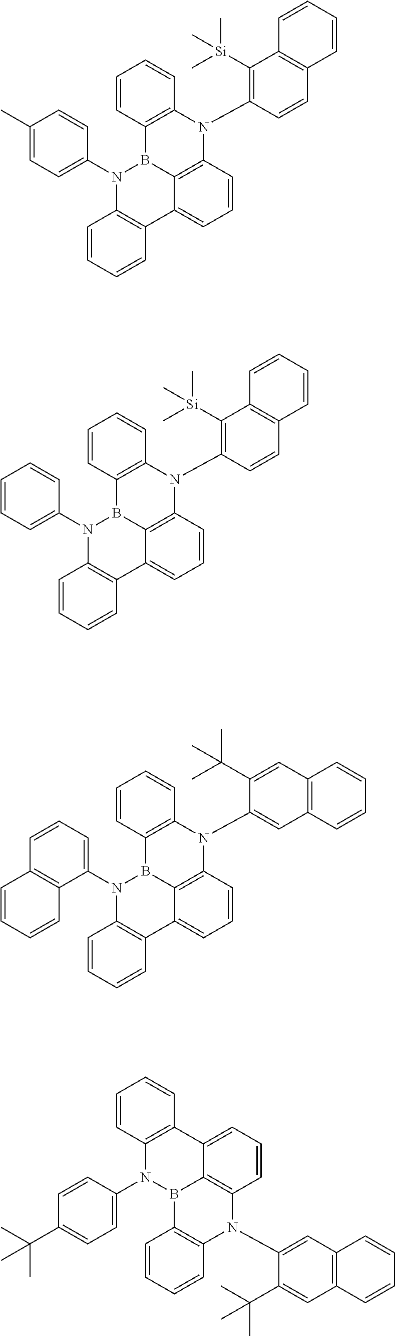 [SMILES: Cc1ccc(N2B3c4ccccc4N(c4ccc5ccccc5c4[Si](C)(C)C)c4cccc(c43)-c3ccccc32)cc1 ; C[Si](C)(C)c1c(N2c3ccccc3B3c4c(cccc42)-c2ccccc2N3c2ccccc2)ccc2ccccc12 ; CC(C)(C)c1cc2ccccc2cc1N1c2ccccc2B2c3c(cccc31)-c1ccccc1N2c1cccc2ccccc12 ; CC(C)(C)c1ccc(N2B3c4ccccc4N(c4cc5ccccc5cc4C(C)(C)C)c4cccc(c43)-c3ccccc32)cc1]

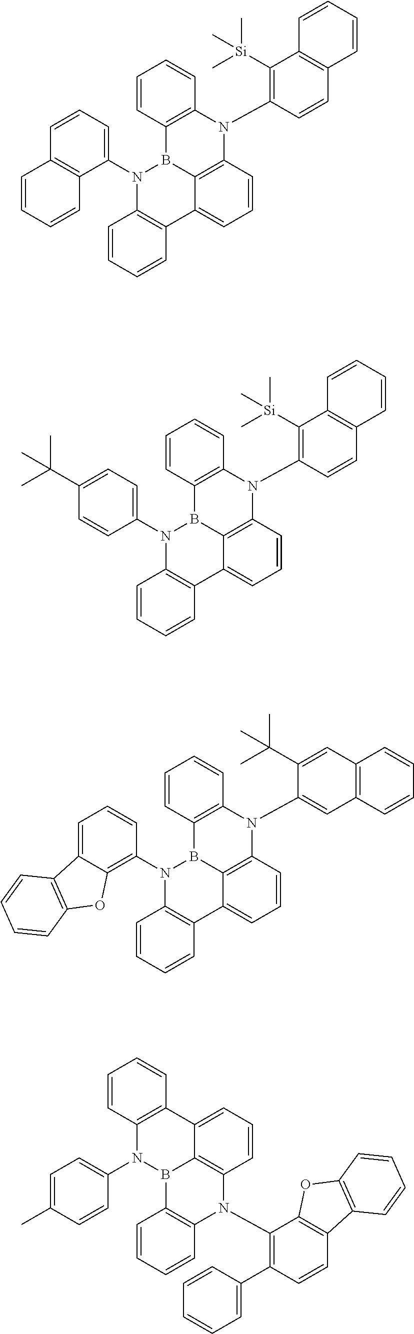 [SMILES: C[Si](C)(C)c1c(N2c3ccccc3B3c4c(cccc42)-c2ccccc2N3c2cccc3ccccc23)ccc2ccccc12 ; CC(C)(C)c1ccc(N2B3c4ccccc4N(c4ccc5ccccc5c4[Si](C)(C)C)c4cccc(c43)-c3ccccc32)cc1 ; CC(C)(C)c1cc2ccccc2cc1N1c2ccccc2B2c3c(cccc31)-c1ccccc1N2c1cccc2c1oc1ccccc12 ; Cc1ccc(N2B3c4ccccc4N(c4c(-c5ccccc5)ccc5c4oc4ccccc45)c4cccc(c43)-c3ccccc32)cc1]

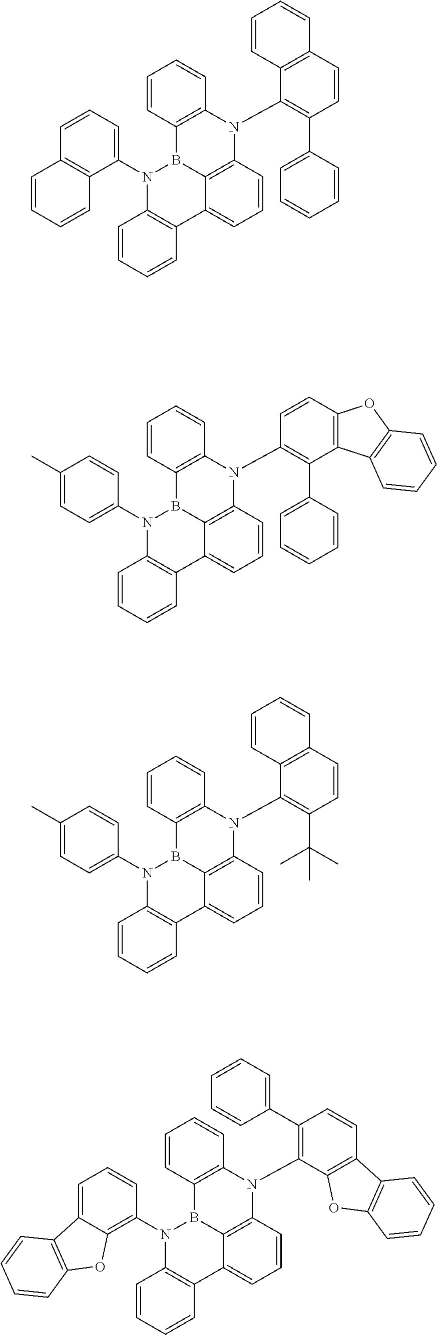 [SMILES: c1ccc(-c2ccc3ccccc3c2N2c3ccccc3B3c4c(cccc42)-c2ccccc2N3c2cccc3ccccc23)cc1 ; Cc1ccc(N2B3c4ccccc4N(c4ccc5oc6ccccc6c5c4-c4ccccc4)c4cccc(c43)-c3ccccc32)cc1 ; Cc1ccc(N2B3c4ccccc4N(c4c(C(C)(C)C)ccc5ccccc45)c4cccc(c43)-c3ccccc32)cc1 ; c1ccc(-c2ccc3c(oc4ccccc43)c2N2c3ccccc3B3c4c(cccc42)-c2ccccc2N3c2cccc3c2oc2ccccc23)cc1]

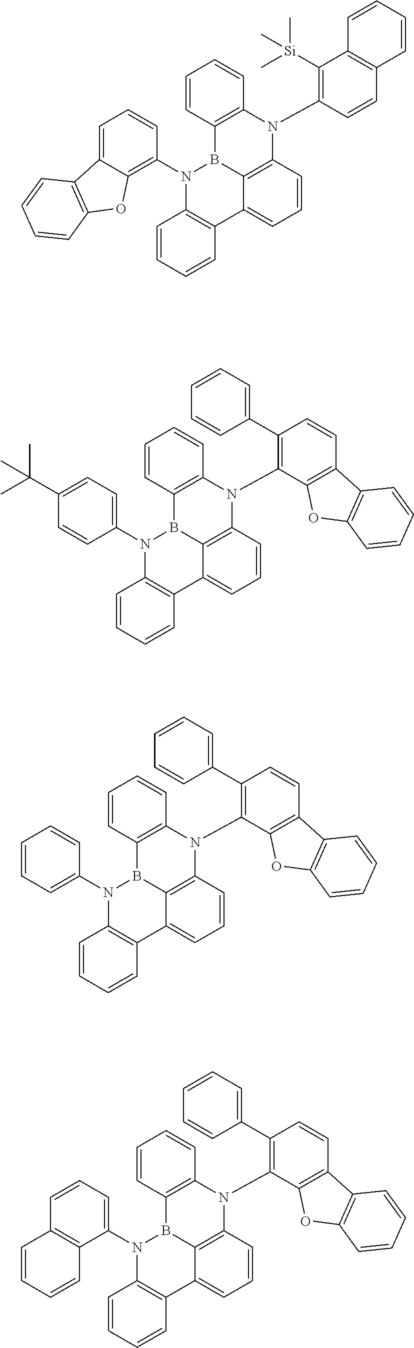 [SMILES: C[Si](C)(C)c1c(N2c3ccccc3B3c4c(cccc42)-c2ccccc2N3c2cccc3c2oc2ccccc23)ccc2ccccc12 ; CC(C)(C)c1ccc(N2B3c4ccccc4N(c4c(-c5ccccc5)ccc5c4oc4ccccc45)c4cccc(c43)-c3ccccc32)cc1 ; c1ccc(-c2ccc3c(oc4ccccc43)c2N2c3ccccc3B3c4c(cccc42)-c2ccccc2N3c2ccccc2)cc1 ; c1ccc(-c2ccc3c(oc4ccccc43)c2N2c3ccccc3B3c4c(cccc42)-c2ccccc2N3c2cccc3ccccc23)cc1]

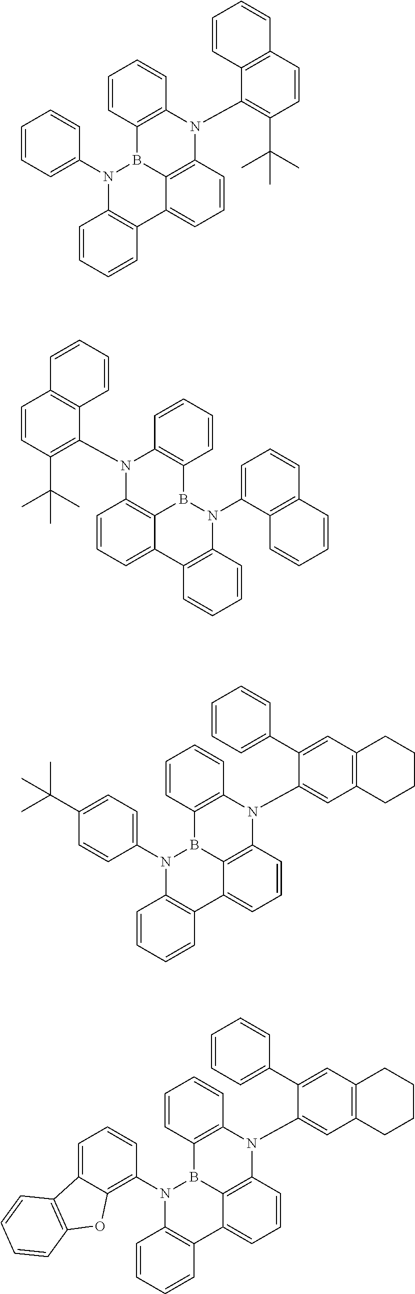

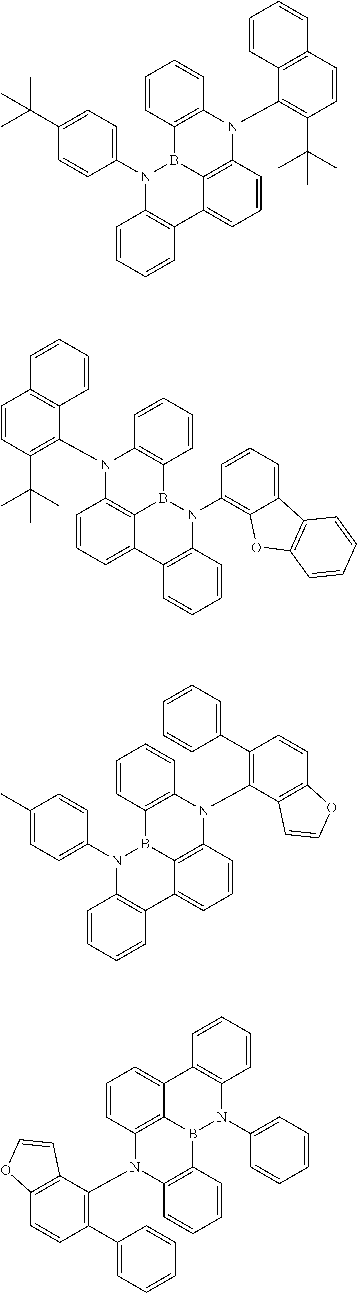

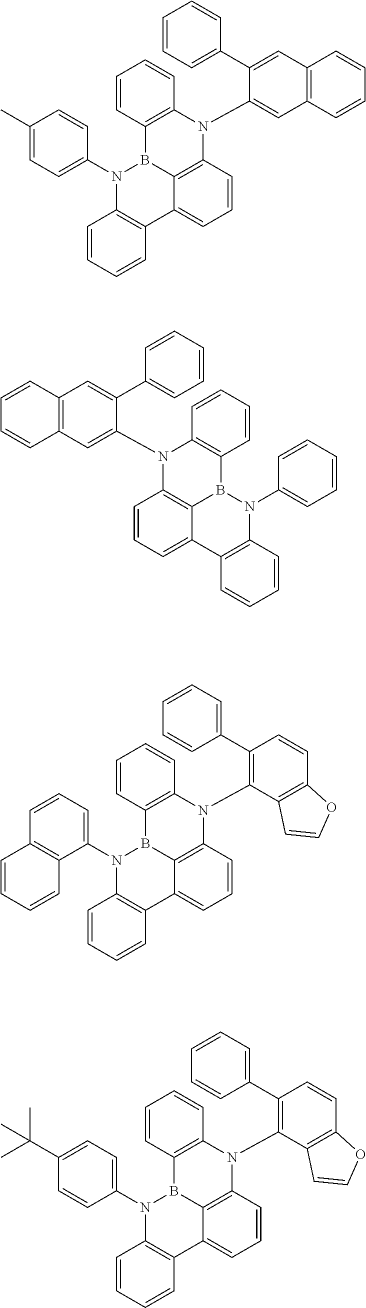 [SMILES: Cc1ccc(N2B3c4ccccc4N(c4cc5ccccc5cc4-c4ccccc4)c4cccc(c43)-c3ccccc32)cc1 ; c1ccc(-c2cc3ccccc3cc2N2c3ccccc3B3c4c(cccc42)-c2ccccc2N3c2ccccc2)cc1 ; c1ccc(-c2ccc3occc3c2N2c3ccccc3B3c4c(cccc42)-c2ccccc2N3c2cccc3ccccc23)cc1 ; CC(C)(C)c1ccc(N2B3c4ccccc4N(c4c(-c5ccccc5)ccc5occc45)c4cccc(c43)-c3ccccc32)cc1]

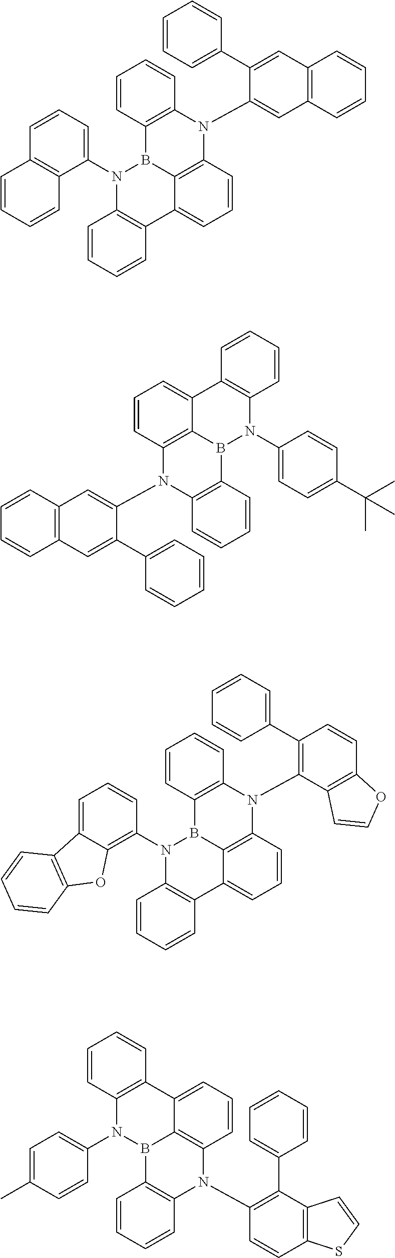

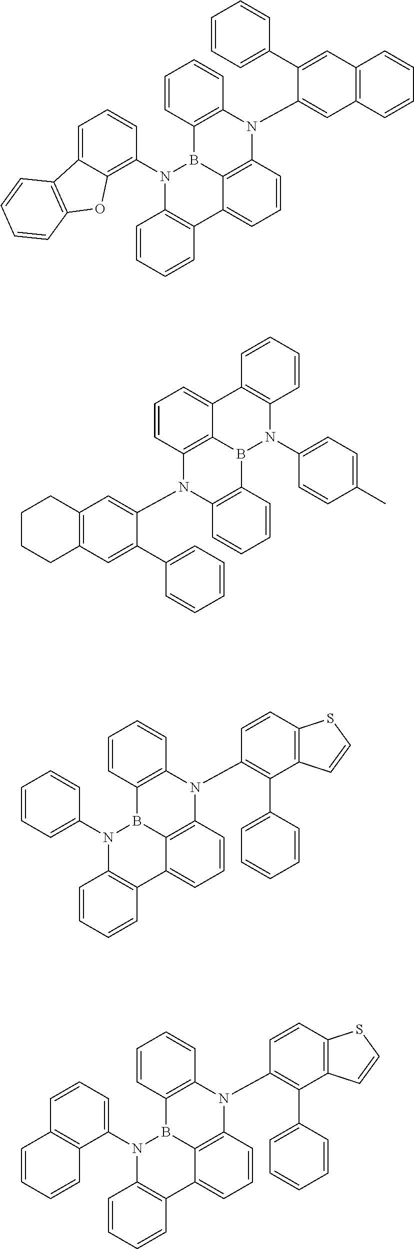 [SMILES: c1ccc(-c2cc3ccccc3cc2N2c3ccccc3B3c4c(cccc42)-c2ccccc2N3c2cccc3c2oc2ccccc23)cc1 ; Cc1ccc(N2B3c4ccccc4N(c4cc5c(cc4-c4ccccc4)CCCC5)c4cccc(c43)-c3ccccc32)cc1 ; c1ccc(-c2c(N3c4ccccc4B4c5c(cccc53)-c3ccccc3N4c3ccccc3)ccc3sccc23)cc1 ; c1ccc(-c2c(N3c4ccccc4B4c5c(cccc53)-c3ccccc3N4c3cccc4ccccc34)ccc3sccc23)cc1]

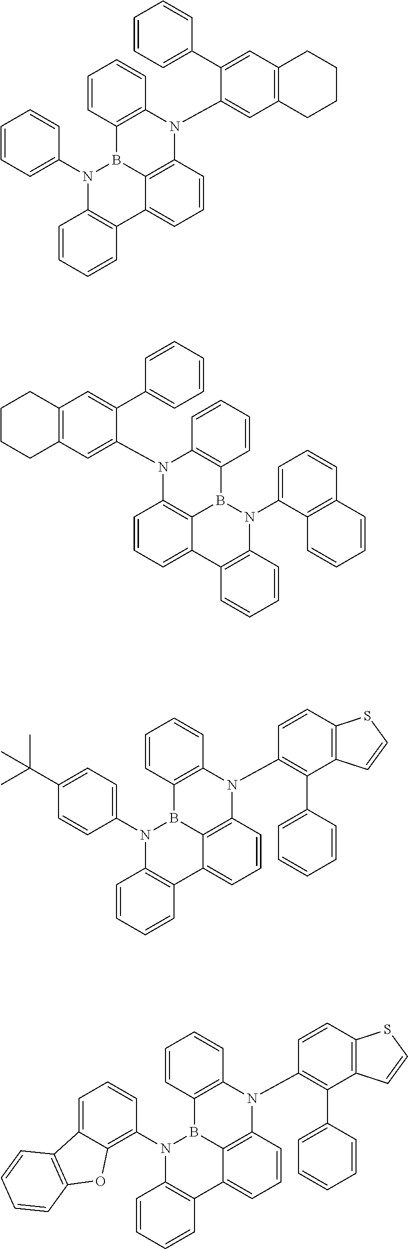 [SMILES: c1ccc(-c2cc3c(cc2N2c4ccccc4B4c5c(cccc52)-c2ccccc2N4c2ccccc2)CCCC3)cc1 ; c1ccc(-c2cc3c(cc2N2c4ccccc4B4c5c(cccc52)-c2ccccc2N4c2cccc4ccccc24)CCCC3)cc1 ; CC(C)(C)c1ccc(N2B3c4ccccc4N(c4ccc5sccc5c4-c4ccccc4)c4cccc(c43)-c3ccccc32)cc1 ; c1ccc(-c2c(N3c4ccccc4B4c5c(cccc53)-c3ccccc3N4c3cccc4c3oc3ccccc34)ccc3sccc23)cc1]

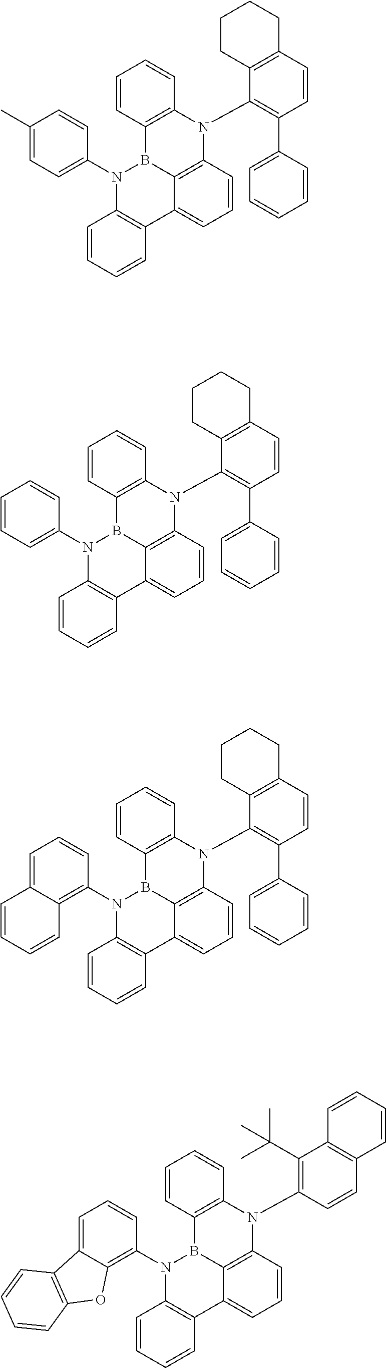 [SMILES: Cc1ccc(N2B3c4ccccc4N(c4c(-c5ccccc5)ccc5c4CCCC5)c4cccc(c43)-c3ccccc32)cc1 ; c1ccc(-c2ccc3c(c2N2c4ccccc4B4c5c(cccc52)-c2ccccc2N4c2ccccc2)CCCC3)cc1 ; c1ccc(-c2ccc3c(c2N2c4ccccc4B4c5c(cccc52)-c2ccccc2N4c2cccc4ccccc24)CCCC3)cc1 ; CC(C)(C)c1c(N2c3ccccc3B3c4c(cccc42)-c2ccccc2N3c2cccc3c2oc2ccccc23)ccc2ccccc12]

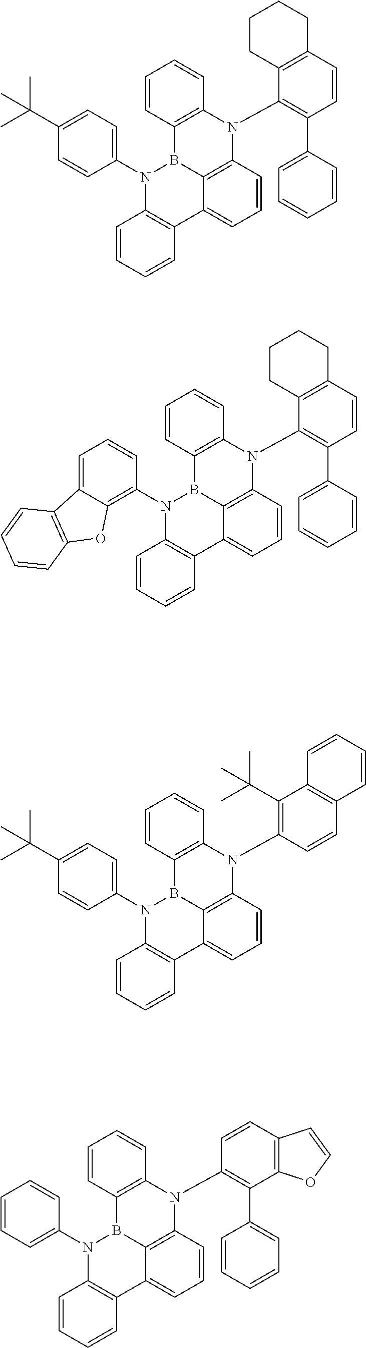 [SMILES: CC(C)(C)c1ccc(N2B3c4ccccc4N(c4c(-c5ccccc5)ccc5c4CCCC5)c4cccc(c43)-c3ccccc32)cc1 ; c1ccc(-c2ccc3c(c2N2c4ccccc4B4c5c(cccc52)-c2ccccc2N4c2cccc4c2oc2ccccc24)CCCC3)cc1 ; CC(C)(C)c1ccc(N2B3c4ccccc4N(c4ccc5ccccc5c4C(C)(C)C)c4cccc(c43)-c3ccccc32)cc1 ; c1ccc(-c2c(N3c4ccccc4B4c5c(cccc53)-c3ccccc3N4c3ccccc3)ccc3ccoc23)cc1]

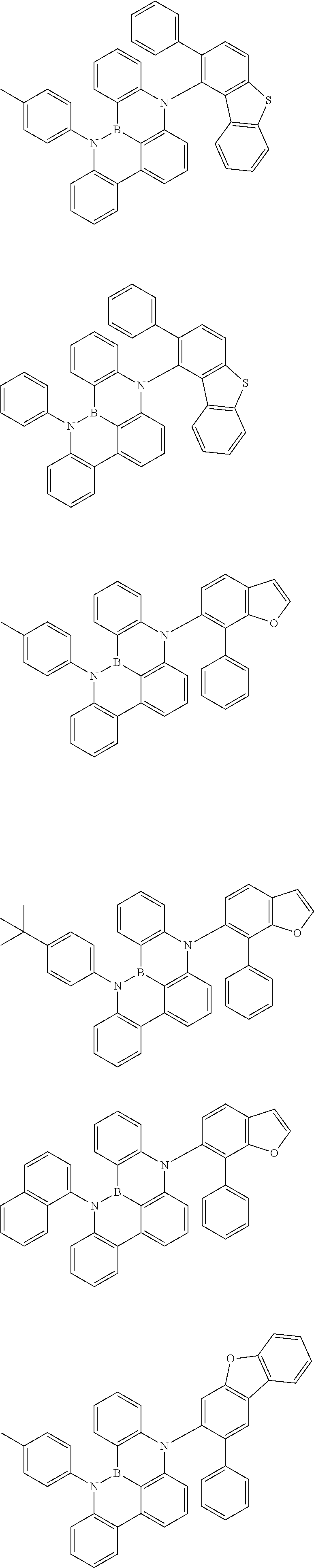 [SMILES: Cc1ccc(N2B3c4ccccc4N(c4c(-c5ccccc5)ccc5sc6ccccc6c45)c4cccc(c43)-c3ccccc32)cc1 ; c1ccc(-c2ccc3sc4ccccc4c3c2N2c3ccccc3B3c4c(cccc42)-c2ccccc2N3c2ccccc2)cc1 ; Cc1ccc(N2B3c4ccccc4N(c4ccc5ccoc5c4-c4ccccc4)c4cccc(c43)-c3ccccc32)cc1 ; CC(C)(C)c1ccc(N2B3c4ccccc4N(c4ccc5ccoc5c4-c4ccccc4)c4cccc(c43)-c3ccccc32)cc1 ; c1ccc(-c2c(N3c4ccccc4B4c5c(cccc53)-c3ccccc3N4c3cccc4ccccc34)ccc3ccoc23)cc1 ; Cc1ccc(N2B3c4ccccc4N(c4cc5oc6ccccc6c5cc4-c4ccccc4)c4cccc(c43)-c3ccccc32)cc1]

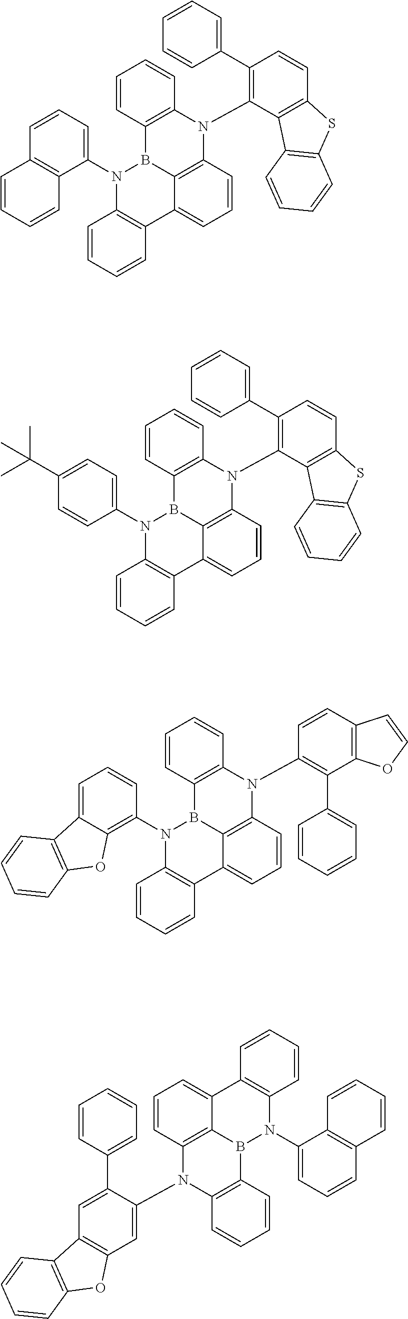 [SMILES: c1ccc(-c2ccc3sc4ccccc4c3c2N2c3ccccc3B3c4c(cccc42)-c2ccccc2N3c2cccc3ccccc23)cc1 ; CC(C)(C)c1ccc(N2B3c4ccccc4N(c4c(-c5ccccc5)ccc5sc6ccccc6c45)c4cccc(c43)-c3ccccc32)cc1 ; c1ccc(-c2c(N3c4ccccc4B4c5c(cccc53)-c3ccccc3N4c3cccc4c3oc3ccccc34)ccc3ccoc23)cc1 ; c1ccc(-c2cc3c(cc2N2c4ccccc4B4c5c(cccc52)-c2ccccc2N4c2cccc4ccccc24)oc2ccccc23)cc1]

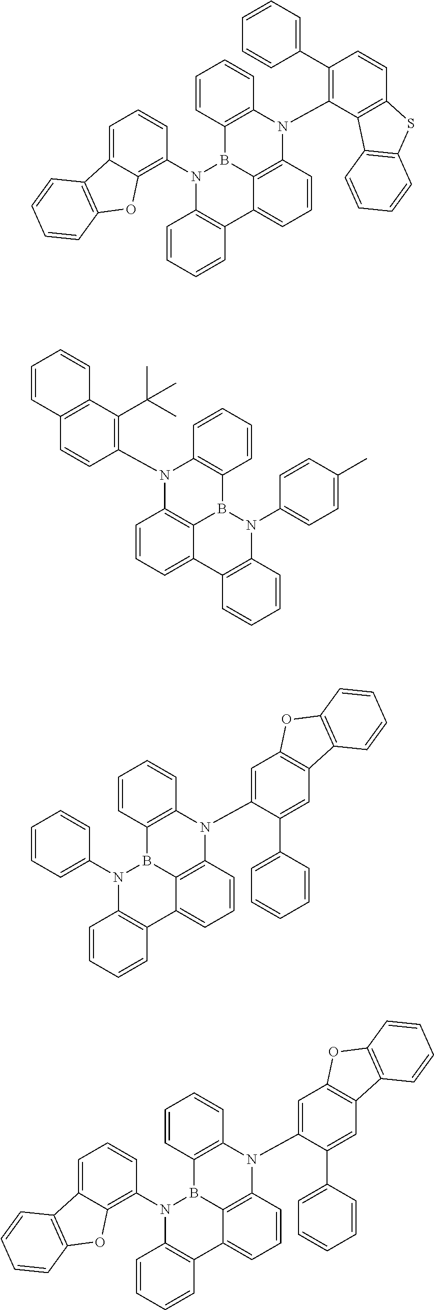 [SMILES: c1ccc(-c2ccc3sc4ccccc4c3c2N2c3ccccc3B3c4c(cccc42)-c2ccccc2N3c2cccc3c2oc2ccccc23)cc1 ; Cc1ccc(N2B3c4ccccc4N(c4ccc5ccccc5c4C(C)(C)C)c4cccc(c43)-c3ccccc32)cc1 ; c1ccc(-c2cc3c(cc2N2c4ccccc4B4c5c(cccc52)-c2ccccc2N4c2ccccc2)oc2ccccc23)cc1 ; c1ccc(-c2cc3c(cc2N2c4ccccc4B4c5c(cccc52)-c2ccccc2N4c2cccc4c2oc2ccccc24)oc2ccccc23)cc1]

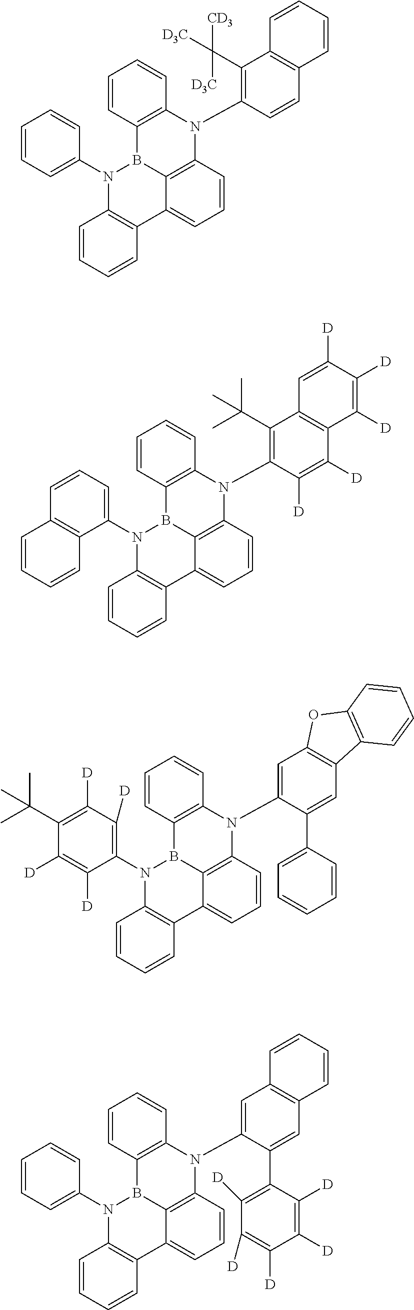 [SMILES: [2H]C([2H])([2H])C(c1c(N2c3ccccc3B3c4c(cccc42)-c2ccccc2N3c2ccccc2)ccc2ccccc12)(C([2H])([2H])[2H])C([2H])([2H])[2H] ; [2H]c1cc2c(C(C)(C)C)c(N3c4ccccc4B4c5c(cccc53)-c3ccccc3N4c3cccc4ccccc34)c([2H])c([2H])c2c([2H])c1[2H] ; [2H]c1c([2H])c(C(C)(C)C)c([2H])c([2H])c1N1B2c3ccccc3N(c3cc4oc5ccccc5c4cc3-c3ccccc3)c3cccc(c32)-c2ccccc21 ; [2H]c1c([2H])c([2H])c(-c2cc3ccccc3cc2N2c3ccccc3B3c4c(cccc42)-c2ccccc2N3c2ccccc2)c([2H])c1[2H]]

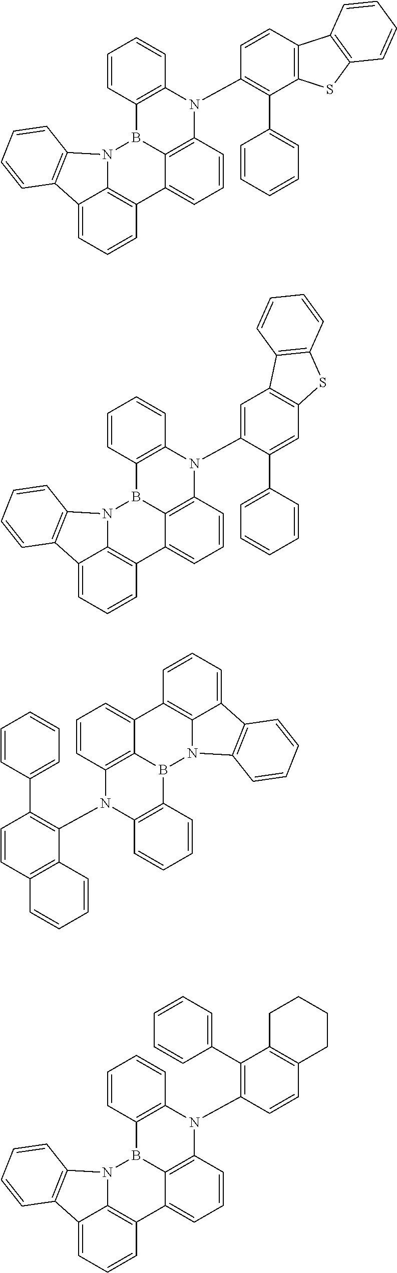 [SMILES: c1ccc(-c2c(N3c4ccccc4B4c5c(cccc53)-c3cccc5c6ccccc6n4c35)ccc3c2sc2ccccc23)cc1 ; c1ccc(-c2cc3sc4ccccc4c3cc2N2c3ccccc3B3c4c(cccc42)-c2cccc4c5ccccc5n3c24)cc1 ; c1ccc(-c2ccc3ccccc3c2N2c3ccccc3B3c4c(cccc42)-c2cccc4c5ccccc5n3c24)cc1 ; c1ccc(-c2c(N3c4ccccc4B4c5c(cccc53)-c3cccc5c6ccccc6n4c35)ccc3c2CCCC3)cc1]

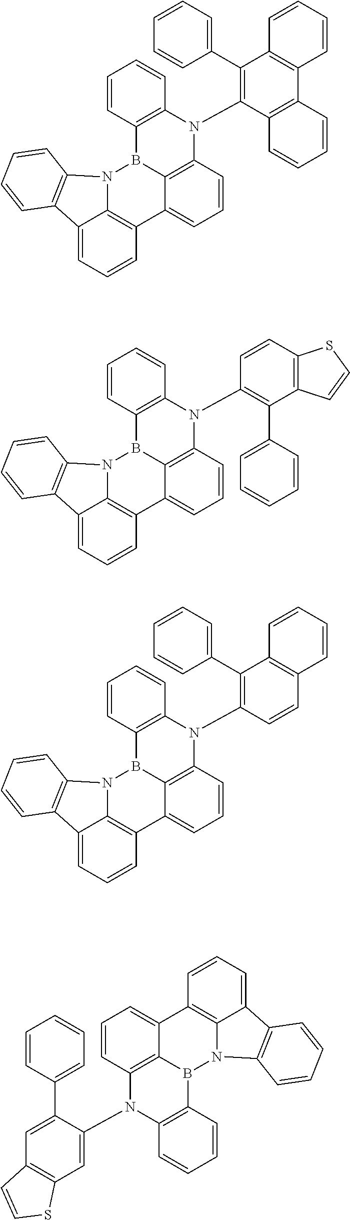 [SMILES: c1ccc(-c2c(N3c4ccccc4B4c5c(cccc53)-c3cccc5c6ccccc6n4c35)c3ccccc3c3ccccc23)cc1 ; c1ccc(-c2c(N3c4ccccc4B4c5c(cccc53)-c3cccc5c6ccccc6n4c35)ccc3sccc23)cc1 ; c1ccc(-c2c(N3c4ccccc4B4c5c(cccc53)-c3cccc5c6ccccc6n4c35)ccc3ccccc23)cc1 ; c1ccc(-c2cc3ccsc3cc2N2c3ccccc3B3c4c(cccc42)-c2cccc4c5ccccc5n3c24)cc1]

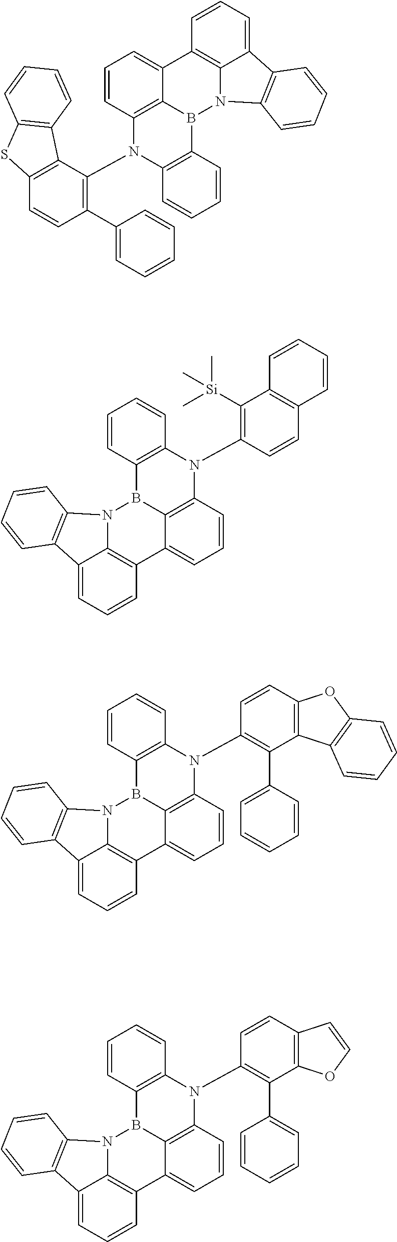 [SMILES: c1ccc(-c2ccc3sc4ccccc4c3c2N2c3ccccc3B3c4c(cccc42)-c2cccc4c5ccccc5n3c24)cc1 ; C[Si](C)(C)c1c(N2c3ccccc3B3c4c(cccc42)-c2cccc4c5ccccc5n3c24)ccc2ccccc12 ; c1ccc(-c2c(N3c4ccccc4B4c5c(cccc53)-c3cccc5c6ccccc6n4c35)ccc3oc4ccccc4c23)cc1 ; c1ccc(-c2c(N3c4ccccc4B4c5c(cccc53)-c3cccc5c6ccccc6n4c35)ccc3ccoc23)cc1]

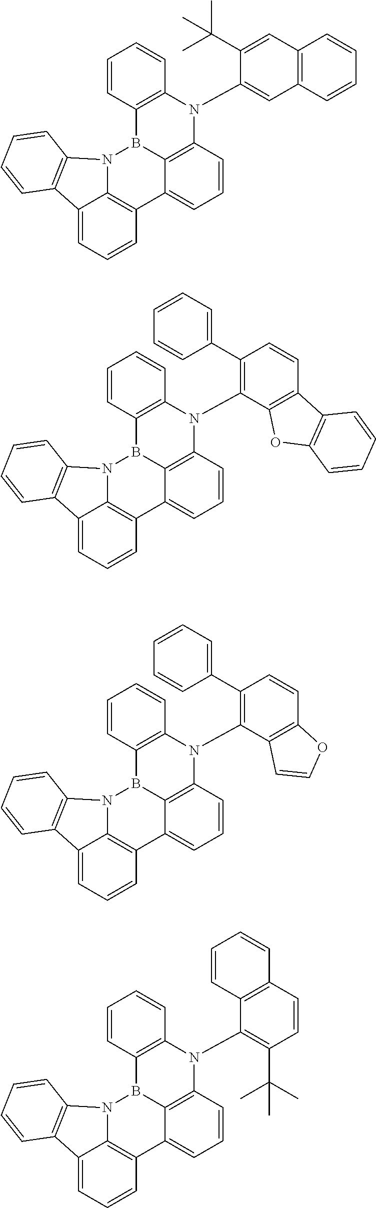 [SMILES: CC(C)(C)c1cc2ccccc2cc1N1c2ccccc2B2c3c(cccc31)-c1cccc3c4ccccc4n2c13 ; c1ccc(-c2ccc3c(oc4ccccc43)c2N2c3ccccc3B3c4c(cccc42)-c2cccc4c5ccccc5n3c24)cc1 ; c1ccc(-c2ccc3occc3c2N2c3ccccc3B3c4c(cccc42)-c2cccc4c5ccccc5n3c24)cc1 ; CC(C)(C)c1ccc2ccccc2c1N1c2ccccc2B2c3c(cccc31)-c1cccc3c4ccccc4n2c13]

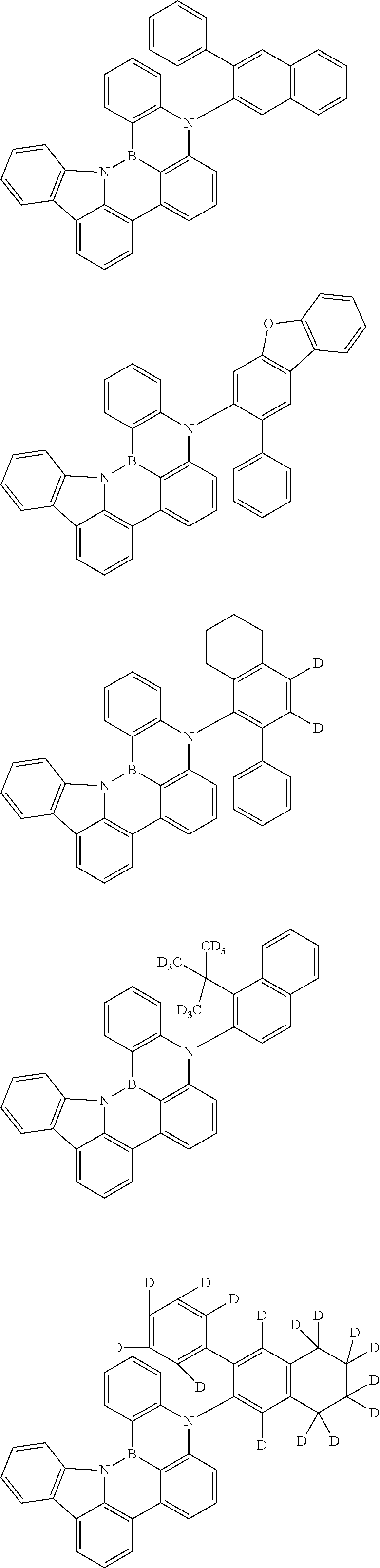

### [Material for Organic EL Device]

The compound according to an aspect of the present invention is useful as a material for an organic EL device, is useful as a material for an emitting layer of an organic EL device, and is particularly useful as a dopant material for an emitting layer.

When the compound according to an aspect of the present invention is used for an emitting layer of an organic EL device, an organic EL device having high chromatic purity and long lifetime without shortening emission wavelength therefrom can be obtained.

### [Organic EL Device]

An organic EL device according to an aspect of the present invention includes a cathode; an anode; and at least one organic layer arranged between the cathode and the anode, wherein at least one layer of the at least one organic layer includes the compound represented by the formula (1).

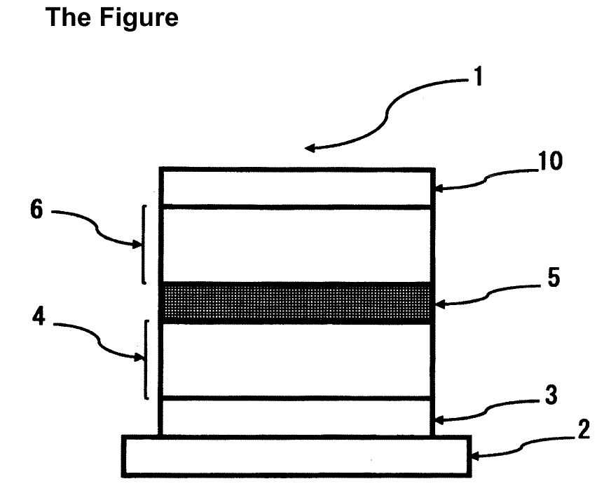

A schematic configuration of the organic EL device according to an aspect of the present invention will be described with reference to FIG. 1.

The organic EL device 1 according to an aspect of the present invention includes a substrate 2, an anode 3, an emitting layer 5 being an organic layer, a cathode 10, an organic layer 4 between the anode 3 and the emitting layer 5, and an organic layer 6 between the emitting layer 5 and the cathode 10.

Each of the organic layer 4 and the organic layer 6 may be a single layer or may composed of a plurality of layers.

Further, the organic layer 4 may include a hole-transporting region. The hole-transporting region may include a hole-injecting layer, a hole-transporting layer, an electron-barrier layer and the like. The organic layer 6 may include an electron-transporting region. The electron-transporting region may include an electron-injecting layer, an electron-transporting layer, a hole-barrier layer and the like.

The compound represented by the formula (1) is contained in the organic layer 4, the emitting layer 5 or the organic layer 6. In one embodiment, the compound represented by the formula (1) is included in the emitting layer 5. The compound represented by the formula (1) can function as a dopant material in the emitting layer 5.

In the organic EL device according to an aspect of the present invention, the at least one layer of the at least one organic layer includes a first compound and a second compound, and the first compound is the compound represented by the formula (1). The first compound and the second compound are different compounds.

In one embodiment, the second compound is a heterocyclic compound or a fused aromatic compound.

In one embodiment, the second compound is an anthracene derivative.

In one embodiment, the second compound is a compound represented by the following formula (10).

### <Compound Represented by the Formula (10) >

A compound represented by formula (10) will be described.

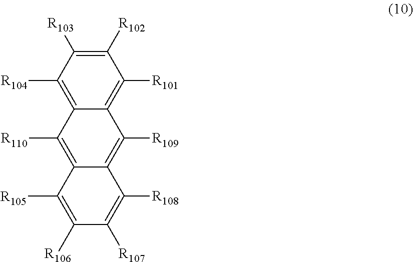

In the formula (10),
one or more sets of the adjacent two or more of R₁₀₁ to R₁₁₀ form a substituted or unsubstituted, saturated or unsaturated ring, or do not form the substituted or unsubstituted, saturated or unsaturated ring;
R₁₀₁ to R₁₁₀ which do not form the substituted or unsubstituted, saturated or unsaturated ring
are independently
a hydrogen atom,
a substituent R, or
a group represented by the following formula (11):

—L₁₀₁-Ar₁₀₁ (11).

In the formula (11),
L₁₀₁ is
a single bond,
a substituted or unsubstituted arylene group having 6 to 50 ring carbon atoms, or
a substituted or unsubstituted divalent heterocyclic group having 5 to 50 ring atoms;
Ar₁₀₁ is
a substituted or unsubstituted aryl group having 6 to 50 ring carbon atoms, or
a substituted or unsubstituted monovalent heterocyclic group having 5 to 50 ring atoms;
the substituent R is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₀)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group having 6 to 50 ring carbon atoms, or
a substituted or unsubstituted monovalent heterocyclic group having 5 to 50 ring atoms;
when two or more substituents R are present, the two or more substituents R may be the same as or different from each other;
R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms.
when two or more of each of R₉₀₁ to R₉₀₇ are present, the two or more of each of R₉₀₁ to R₉₀₇ may be the same as or different from each other;
here, at least one of R₁₀₁ to R₁₁₀ which does not form the substituted or unsubstituted, saturated or unsaturated ring is the group represented by the formula (11); when two or more groups represented by the formula (11) are present, each of the two or more groups represented by the formula (11) may be the same as or different from each other.

The compound represented by the formula (10) may have a deuterium atom as a hydrogen atom.

In one embodiment, at least one of Ar₁₀₁ in the formula (10) is a substituted or unsubstituted aryl group having 6 to 50 ring carbon atoms.

In one embodiment, at least one of Ar₁₀₁ in the formula (10) is a substituted or unsubstituted monovalent heterocyclic group having 5 to 50 ring atoms.

In one embodiment, all of Ar₁₀₁'s in the formula (10) are a substituted or unsubstituted aryl group having 6 to 50 ring carbon atoms. The plurality of Ar₁₀₁'s may be the same as or different from each other.

In one embodiment, one of Ar₁₀₁ in the formula (10) is a substituted or unsubstituted monovalent heterocyclic group having 5 to 50 ring atoms, and the remaining Ar₁₀₁ is a substituted or unsubstituted aryl group having 6 to 50 ring carbon atoms. The plurality of Ar₁₀₁'s may be the same as or different from each other.

In one embodiment, at least one of L₁₀₁ in the formula (10) is a single bond.

In one embodiment, all of L₁₀₁ in the formula (10) are single bonds.

In one embodiment, at least one of L₁₀₁ in the formula (10) is a substituted or unsubstituted arylene group having 6 to 50 ring carbon atoms.

In one embodiment, at least one of L₁₀₁ in the formula (10) is a substituted or unsubstituted phenylene group, or a substituted or unsubstituted naphthyl group.

In one embodiment, the group represented by -L₁₀₁-Ar₁₀₁ in the formula (10) is selected from the group consisting of
a substituted or unsubstituted phenyl group,
a substituted or unsubstituted naphthyl group,
a substituted or unsubstituted biphenyl group,
a substituted or unsubstituted phenanthrenyl group,
a substituted or unsubstituted benzophenanthrenyl group,
a substituted or unsubstituted fluorenyl group,
a substituted or unsubstituted benzofluorenyl group,
a substituted or unsubstituted dibenzofuranyl group,
a substituted or unsubstituted naphthobenzofuranyl group,
a substituted or unsubstituted dibenzothiophenyl group, and
a substituted or unsubstituted carbazolyl group.

In one embodiment, the substituent R in the formula (10) are independently
a substituted or unsubstituted alkyl group having 1 to 50 carbon atoms,
a substituted or unsubstituted cycloalkyl group having 3 to 50 ring carbon atoms,
—Si(R₉₀₀)(R₉₀₂)(R₉₀₃),
—O—(R₉₀₄),
—S—(R₉₀₅),
—N(R₉₀₆)(R₉₀₇),
a halogen atom, a cyano group, a nitro group, or
a substituted or unsubstituted aryl group having 6 to 50 ring carbon atoms.
R₉₀₁ to R₉₀₇ are the same as defined in the formula (10).

In one embodiment, the substituent of “substituted or unsubstituted” in the formula (10) is independently
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₀)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group having 6 to 50 ring carbon atoms, or
a substituted or unsubstituted monovalent heterocyclic group having 5 to 50 ring atoms.
R₉₀₁ to R₉₀₇ are the same as defined in the formula (10).

In one embodiment, the substituent of “substituted or unsubstituted” in the formula (10) is independently
a substituted or unsubstituted alkyl group having 1 to 50 carbon atoms,
a substituted or unsubstituted cycloalkyl group having 3 to 50 ring carbon atoms,
—Si(R₉₀₀)(R₉₀₂)(R₉₀₃),
—O—(R₉₀₄),
—S—(R₉₀₅),
—N(R₉₀₆)(R₉₀₇),
a halogen atom, a cyano group, a nitro group, or
a substituted or unsubstituted aryl group having 6 to 50 ring carbon atoms.
R₉₀₁ to R₉₀₇ are the same as defined in the formula (10).

In one embodiment, the substituent in the case of “substituted or unsubstituted” in the formula (10) is selected from the group consisting of
an alkyl group having 1 to 18 carbon atoms,
an aryl group having 6 to 18 ring carbon atoms, and
a monovalent heterocyclic group having 5 to 18 ring atoms.

In one embodiment, the substituent in the case of “substituted or unsubstituted” in the formula (10) is an alkyl group having 1 to 5 carbon atoms.

In one embodiment, the compound represented by the formula (10) is a compound represented by the following formula (20).

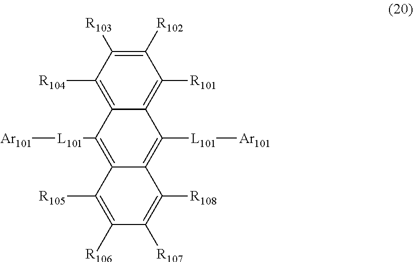

In the formula (20), R₁₀₁ to R₁₀₈, ₁₀₁'s and Ar₁₀ₗ's are the same as defined in the formula (10).

The compound represented by the formula (20) may have a deuterium atom as a hydrogen
atom.

That is, in one embodiment, the compound represented by the formula (10) or the formula (20) has at least two groups represented by the formula (11).

In one embodiment, the compound represented by the formula (10) or the formula (20) has two or three groups represented by the formula (11).

In one embodiment, R₁₀₁ to R₁₁₀ in formulas (10) and (20) do not form the substituted or unsubstituted, saturated or unsaturated ring.

In one embodiment, R₁₀₁ to R₁₁₀ in the formulas (10) and (20) is a hydrogen atom.

In one embodiment, the compound represented by the formula (20) is a compound represented by the following formula (30).

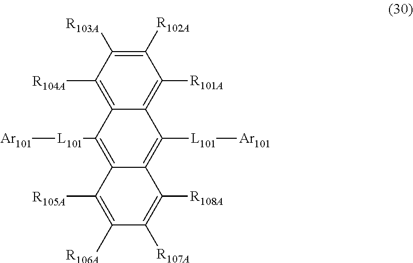

In the formula (30), L₁₀₁'s and Ar₁₀₁'s are the same as defined in the formula (10).

The adjacent two of R_{101A} to R_{108 A} do not form any substituted or unsubstituted, saturated or unsaturated ring.
R_{101A} to R_{108A} are independently
a hydrogen atom, or
a substituent R.

The substituent R is the same as defined in the formula (10).

That is, the compound represented by the formula (30) is a compound having two groups represented by the formula (11).

The compound represented by the formula (30) has substantially only protium atoms as hydrogen atoms.

The expression “having substantially only protium atoms” means the case where the proportion of protium compound based on the total amount of a compound having only protium atoms as hydrogen atoms (protium compound) and a compound having a deuterium atom (deuterium compound), which have the same structure, is 90 mol % or more, 95 mol % or more, or 99 mol % or more.

In one embodiment, the compound represented by the formula (30) is a compound represented by the following formula (31).

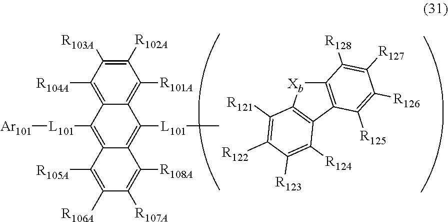

In the formula (31), L₁₀₁'s and Ar₁₀₁ are the same as defined in the formula (10).
R_{101A} to R_{108A} are the same as defined in the formula (30).
X_{b} is O, S, N(R₁₃₁), or C(R₁₃₂)(R₁₃₃).

One of R₁₂₁ to R₁₂₈, and R₁₃₁ to R₁₃₃ is a single bond bonding with L₁₀₁.

One or more sets of the adjacent two or more of R₁₂₁ to R₁₂₈ which are not single bonds bonding with L₁₀₁ form a substituted or unsubstituted, saturated or unsaturated ring, or do not form the substituted or unsubstituted, saturated or unsaturated ring.
R₁₂₁ to R₁₂₈ which are not single bonds bonding with L₁₀₁ and which do not form the substituted or unsubstituted, saturated or unsaturated ring are independently
a hydrogen atom, or
a substituent R.

The substituent R is the same as defined in the formula (10).
R₁₃₁ to R₁₃₃ which are not single bonds bonding with L₁₀₁ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms.

When two or more R₁₃₁ to R₁₃₃ are present, each of the two or more R₁₃₁ to R₁₃₃ may be the same as or different from each other.

In one embodiment, the compound represented by the formula (31) is a compound represented by the following formula (32).

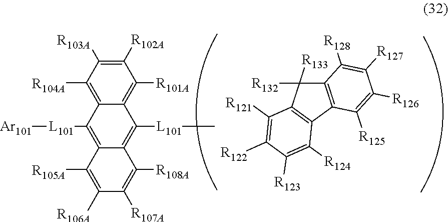

In the formula (32), R_{101A} to R_{108A}, L₁₀₁'s, Ar₁₀₁, R₁₂₁ to R₁₂₈, R₁₃₂ and R₁₃₃ are the same as defined in the formula (31).

In one embodiment, the compound represented by the formula (31) is a compound represented by the following formula (33).

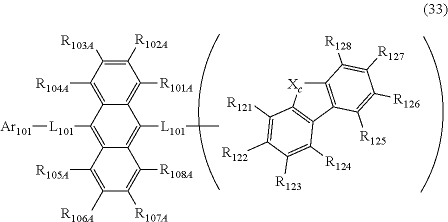

In the formula (33), R_{101A} to R_{108 A}, L₁₀₁'s, Ar₁₀₁, and R₁₂₁ to R₁₂₈ are the same as defined in the formula (31).

X_{c} can be O, S, or NR₁₃₁.

R₁₃₁ is the same as defined in the formula (31).

In one embodiment, the compound represented by the formula (31) is a compound represented by the following formula (34).

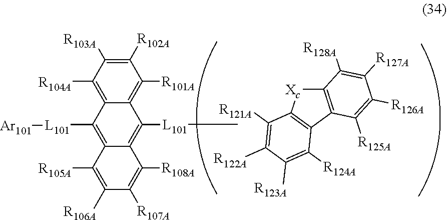

In the formula (34), R_{101A} to R_{108 A}, 1-₁₀₁'s and Ar₁₀₁ are the same as defined in the formula (31).
X_{c} is 0, S or NR₁₃₁.
R₁₃₁ is the same as defined in the formula (31).
One of R_{121A} to R_{128A} is a single bond bonding with L₁₀₁.
One or more sets of the adjacent two or more of R_{121A} to R_{128A} which are not single bonds bonding with L₁₀₁ do not form the substituted or unsubstituted, saturated or unsaturated ring.
R_{121A} to R_{128A} which are not single bonds bonding with L₁₀₁ are independently
a hydrogen atom, or
a substituent R.
The substituent R is the same as defined in the formula (10).

In one embodiment, the compound represented by the formula (31) is a compound represented by the following formula (35).

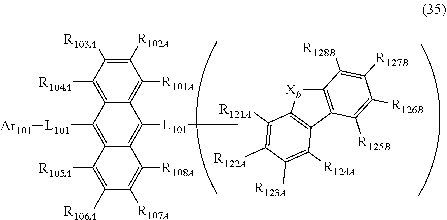

In the formula (35), R_{101A} to R_{108 A}, L₁₀₁'s, Ar₁₀₁ and X_{b} are the same as defined in the formula (31).

One or more sets of the adjacent two or more of R_{121A} to R_{124A} do not form a substituted or unsubstituted, saturated or unsaturated ring by bonding with each other.

Any one set of R_{125B} and R_{126B}, R_{126B} and R_{127B}, and R_{127B} and R_{128B} forms a ring represented by the following formula (35a) or (35b) by bonding with each other.

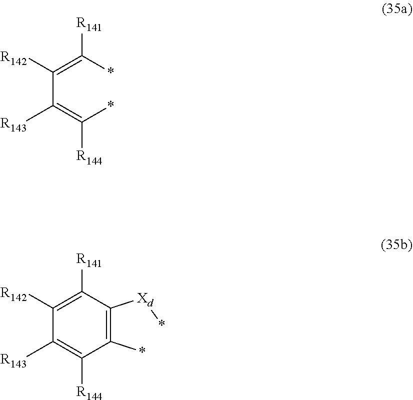

In the formulas (35a) and (35b),
each of two *'s is bonded with each of any one set of R_{125B} and R_{R126B}, R_{126B} and R_{127B}, and R_{127B} and R_{128B}.
R₁₄₁ to R₁₄₄ are independently
a hydrogen atom, or
a substituent R.
The substituent R is the same as defined in the formula (10).
X_{d} is O or S.
One of R_{121A} to R_{124A}, R_{125B} to R_{128B} which do not form the ring represented by the formula (35a) or (35b), and R₁₄₁ to R₁₄₄ is a single bond bonding with L₁₀₁.
R_{121A} to R_{124A} which are not single bonds bonding with L₁₀₁, and R_{125B} to R_{128B} which are not single bonds bonding with L₁₀₁ and which do not form the ring represented by the formula (35a) or (35b) are independently
a hydrogen atom, or
a substituent R.

The substituent R is the same as defined in the formula (10).

In one embodiment, the compound represented by the formula (35) is a compound represented by the following formula (36).

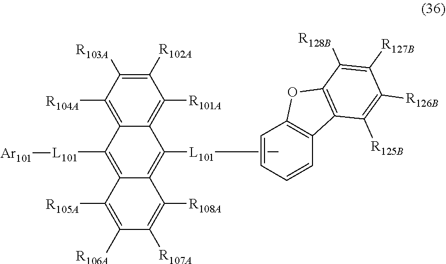

In the formula (36), R_{101A} to R_{108 A}, L₁₀₁'s, Ar₁₀₁, and R_{125B} to R_{128B} are the same as defined in the formula (35).

In one embodiment, the compound represented by the formula (34) is a compound represented by the following formula (37).

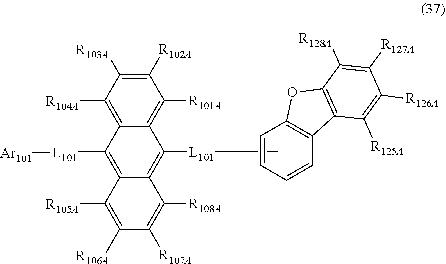

In the formula (37), R_{101A} to R_{108A}, R_{125A} to R_{128A}, L₁₀₁'s and Ar₁O₁ are the same as defined in the formula (34).

In one embodiment, R_{101A} to R_{108 A} in the formulas (30) to (37) are hydrogen atoms.

In one embodiment, the compound represented by the formula (10) is a compound represented by the following formula (40).

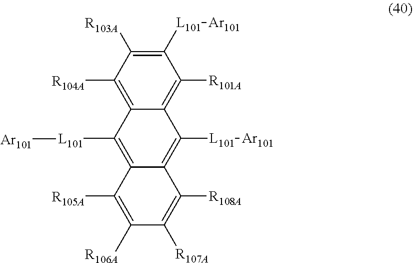

In the formula (40), L₁₀₁'s and Ar₁₀₁'s are the same as defined in the formula (10).
One or more sets of the adjacent two or more of R_{101A}, and R_{103A} to R_{108 A} form a substituted or unsubstituted, saturated or unsaturated ring, or do not form the substituted or unsubstituted, saturated or unsaturated ring.
R_{101A}, and R_{103A} to R_{108 A} which do not form the substituted or unsubstituted, saturated or unsaturated ring are independently
a hydrogen atom, or
a substituent R.
The substituent R is the same as defined in the formula (10).

That is, the compound represented by the formula (40) is a compound having three groups represented by the formula (11). Furthermore, the compound represented by the formula (40) has substantially only protium atoms as hydrogen atoms.

In one embodiment, the compound represented by the formula (40) is represented by the following formula (41).

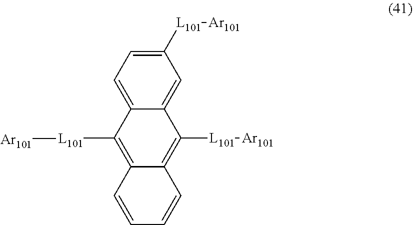

In the formula (41), L₁₀₁'s and Ar₁₀₁'s are the same as defined in the formula (40).

In one embodiment, the compound represented by the formula (40) is a compound represented by any one of the following formulas (42-1) to (42-3).

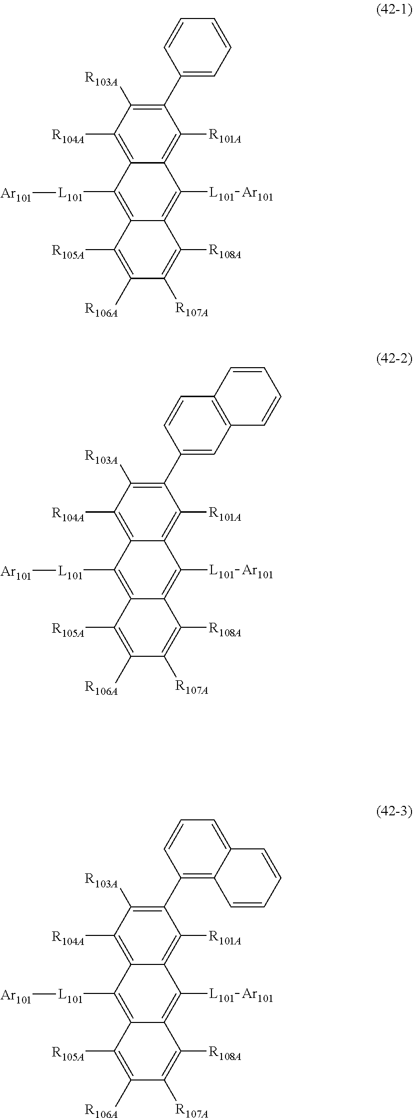

In the formulas (42-1) to (42-3), R_{101A} to R_{108A}, L₁₀₁'s and Ar₁₀₁'s are the same as defined in the formula (40).

In one embodiment, the compounds represented by the formulas (42-1) to (42-3) are a compound represented by any one of the following formulas (43-1) to (43-3).

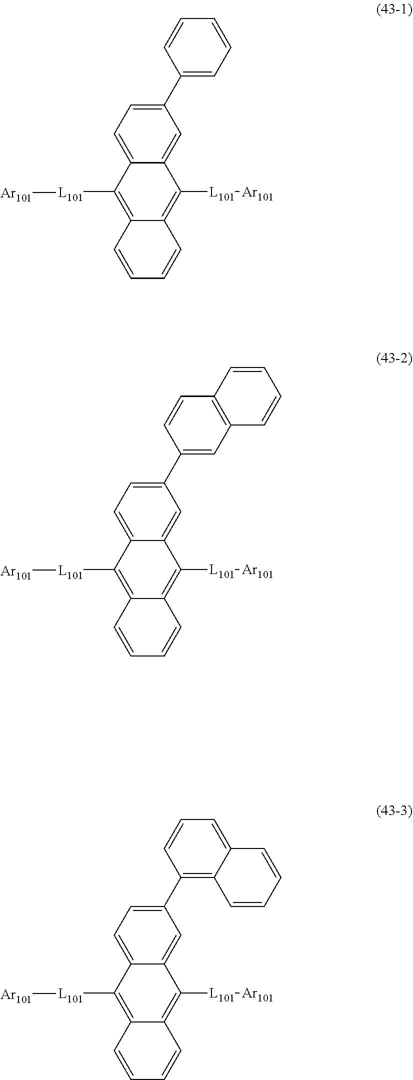

In the formulas (43-1) to (43-3), L₁₀₁'s and Ar₁₀₁'s are the same as defined in the formula (40).

In one embodiment, the group represented by —L₁₀₁-Ar₁₀₁ in the formulas (40), (41), (42-1) to (42-3), and (43-1) to (43-3) is selected from the group consisting of
a substituted or unsubstituted phenyl group,
a substituted or unsubstituted naphthyl group,
a substituted or unsubstituted biphenyl group,
a substituted or unsubstituted phenanthrenyl group,
a substituted or unsubstituted benzophenanthrenyl group,
a substituted or unsubstituted fluorenyl group,
a substituted or unsubstituted benzofluorenyl group,
a substituted or unsubstituted dibenzofuranyl group,
a substituted or unsubstituted naphthobenzofuranyl group,
a substituted or unsubstituted dibenzothiophenyl group, and
a substituted or unsubstituted carbazolyl group.

In one embodiment, the compound represented by the formula (10) or the formula (20) includes a compound in which at least one of the hydrogen atoms possessed by these compounds is a deuterium atom.

In one embodiment, in the formula (20), at least one of,
R₁₀ to R₁₀₈ which are hydrogen atoms,
hydrogen atoms possessed by R₁₀₁ to R₁₀₈ which are the substituents R,
hydrogen atoms possessed by L₁₀₁,
hydrogen atoms possessed by the substituent of L₁₀₁,
hydrogen atoms possessed by Ar₁₀₁, and
hydrogen atoms possessed by the substituent of Ar₁₀₁
is a deuterium atom.

The compounds represented by the formulas (30) to (37) include compounds in which at least one of the hydrogen atoms possessed by these compounds is a deuterium atom.

In one embodiment, at least one of the hydrogen atoms bonding to the carbon atoms constituting the anthracene skeletons in the compounds represented by the formulas (30) to (37) is a deuterium atom.

In one embodiment, the compound represented by the formula (30) is a compound represented by the following formula (30D).

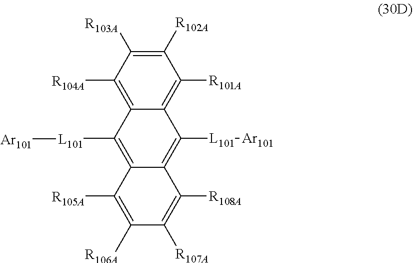

In the formula (30D), R_{101A} to R_{108A}, L₁₀₁'s and Ar₁₀₁'s are the same as defined in the formula (30).

Here, at least one of, R_{101A} to R_{110A} which are hydrogen atoms,
hydrogen atoms possessed by R_{101A} to R_{110A} which are the substituents R,
hydrogen atoms possessed by L₁₀₁,
hydrogen atoms possessed by the substituent of L₁₀₁,
hydrogen atoms possessed by Ar₁₀₁ and
hydrogen atoms possessed by the substituent of Ar₁₀₁
is a deuterium atom.

That is, the compound represented by the formula (30D) is a compound in which at least one of the hydrogen atoms possessed by the compound represented by the formula (30) is a deuterium atom.

In one embodiment, at least one of R_{101A} to R_{108 A} which is a hydrogen atom in the formula (30D) is a deuterium atom.

In one embodiment, the compound represented by the formula (30D) is a compound represented by the following formula (31 D).

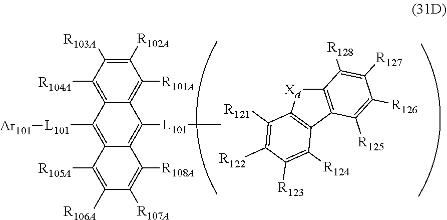

In the formula (31D), R_{101A} to R_{108A}, L₁₀₁'s and Ar₁₀₁ are the same as defined in the formula (30D).
X_{d} is O or S.
One of R₁₂₁ to R₁₂₈ is a single bond bonding with L₁₀₁.
One or more sets of the adjacent two or more of R₁₂₁ to R₁₂₈ which are not single bonds bonding with L₁₀₁ form a substituted or unsubstituted, saturated or unsaturated ring, or do not form the substituted or unsubstituted, saturated or unsaturated ring.
R₁₂₁ to R₁₂₈ which are not a single bond bonding with L₁₀₁ and which do not form the substituted or unsubstituted, saturated or unsaturated ring are independently
a hydrogen atom, or
a substituent R.
The substituent R is the same as defined in the formula (10).
Here, at least one of, R_{101A} to R_{110A} which are hydrogen atoms,
hydrogen atoms possessed by R_{101A} to R_{110A} which are the substituents R,
hydrogen atoms possessed by L₁₀₁,
hydrogen atoms possessed by the substituent of L₁₀₁,
hydrogen atoms possessed by Ar₁₀₁,
hydrogen atoms possessed by the substituent of Ar₁₀₁,
R₁₂₁ to R₁₂₈ which are hydrogen atoms, and
hydrogen atoms possessed by R₁₂₁ to R₁₂₈ which are the substituents R
is a deuterium atom.

In one embodiment, the compound represented by the formula (31D) is a compound represented by the following formula (32D).

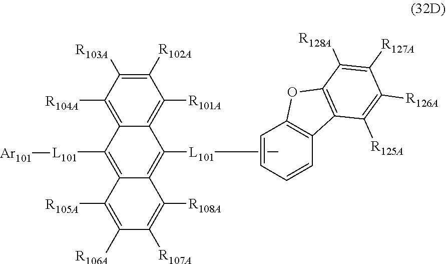

In the formula (32D), R_{101A} to R_{108A}, R_{125A} to R_{128A}, L₁₀₁'s and Ar₁O₁ are the same as defined in the formula (31D).
Here, at least one of,
R_{101A} to R_{108A} which are hydrogen atoms,
hydrogen atoms possessed by R_{101A} to R_{108A} which are the substituents R,
R_{125A} to R_{128A} which are hydrogen atoms,
hydrogen atoms possessed by R_{125A} to R_{128A} which are the substituents R,
hydrogen atoms bonding to the carbon atoms of the dibenzofuran skeleton in the formula (32D),
hydrogen atoms possessed by L₁₀₁,
hydrogen atoms possessed by the substituent of L₁₀₁,
hydrogen atoms possessed by Ar₁₀₁ and
hydrogen atoms possessed by the substituent of Ar₁₀₁
is a deuterium atom.

In one embodiment, the compound represented by the formula (32D) is a compound represented by the following formula (32D-1) or (32D-2).

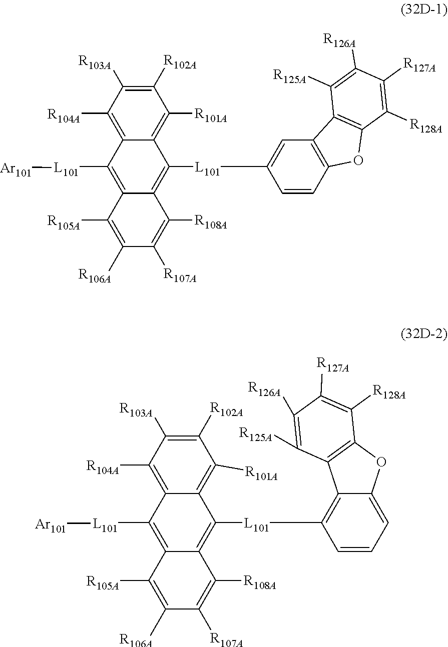

In the formulas (32D-1) and (32D-2), R_{101A} to R_{108A}, R_{125A} to R_{128A}, L₁₀₁'s and Ar₁₀₁ are the same as defined in the formula (32D).

Here, at least one of,
R_{101A} to R_{108A} which are hydrogen atoms,
hydrogen atoms possessed by R_{101A} to R_{108A} which are the substituents R,
R_{125A} to R_{128A} which are hydrogen atoms,
hydrogen atoms possessed by R_{125A} to R_{128A} which are the substituents R,
hydrogen atoms bonding to the carbon atoms of the dibenzofuran skeleton in the formulas (32D-1) and (32D-2),
hydrogen atoms possessed by L₁₀₁,
hydrogen atoms possessed by the substituent of L₁₀₁,
hydrogen atoms possessed by Ar₁₀₁ and
hydrogen atoms possessed by the substituent of Ar₁₀₁
is a deuterium atom.

In one embodiment, at least one of the hydrogen atoms possessed by the compounds represented by the formulas (40), (41), (42-1) to (42-3) and (43-1) to (43-3) is a deuterium atom.

In one embodiment, at least one of the hydrogen atoms (R_{101A} to R_{108 A} which are hydrogen atoms) bonding to the carbon atoms constituting the anthracene skeletons in the compound represented by the formula (41) is a deuterium atom.

In one embodiment, the compound represented by the formula (40) is a compound represented by the following formula (40D).

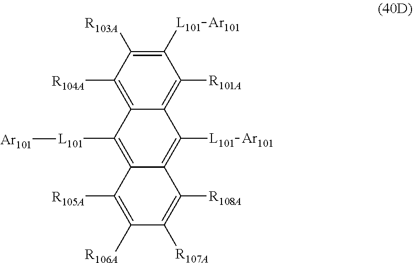

In the formula (40D), L₁₀₁'s and Ar₁₀₁'s are the same as defined in the formula (10).
One or more sets of the adjacent two or more of R_{101A}, and R_{103A} to R_{108 A} do not form the substituted or unsubstituted, saturated or unsaturated ring.
R_{101A}, and R_{103A} to R_{108A} are independent
a hydrogen atom, or
a substituent R.
The substituent R is the same as defined in the formula (10).
Here, at least one of, R_{101A}, and R_{103A} to R_{108A} which are hydrogen atoms,
hydrogen atoms possessed by R_{101A}, and R_{103A} to R_{108 A} which are the substituents R,
hydrogen atoms possessed by L₁₀₁,
hydrogen atoms possessed by the substituent of L₁₀₁,
hydrogen atoms possessed by Ar₁₀₁, and
hydrogen atoms possessed by the substituent of Ar₁₀₁
is a deuterium atom.

In one embodiment, at least one of R_{101A}, and R_{103A} to R_{108A} in the formula (40D) is a deuterium atom.

In one embodiment, the compound represented by the formula (40D) is a compound represented by the following formula (41 D).

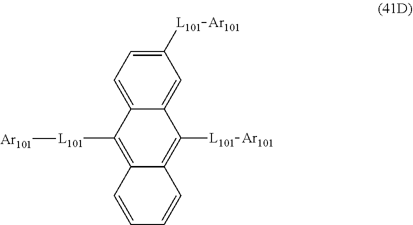

In the formula (41 D), L₁₀₁'s and Ar₁₀₁'s are the same as defined in the formula (40D).
Here, in the formula (41D), at least one of,
hydrogen atoms bonding to the carbon atoms constituting the anthracene skeleton,
hydrogen atoms possessed by L₁₀₁,
hydrogen atoms possessed by the substituent of L₁₀₁,
hydrogen atoms possessed by Ar₁₀₁, and
hydrogen atoms possessed by the substituent of Ar₁₀₁
is a deuterium atom.

In one embodiment, the compound represented by the formula (40D) is a compound represented by any one of the following formulas (42D-1) to (42D-3).

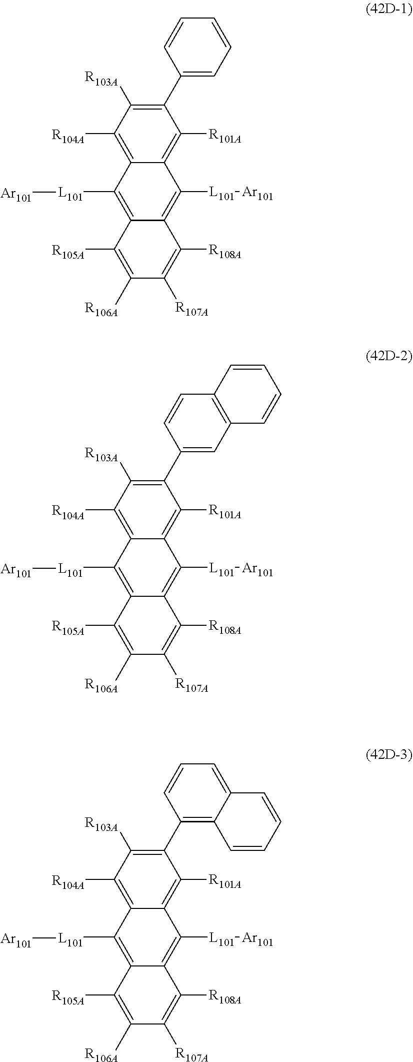

In the formula (42D-1) to (42D-3), R_{101A} to R_{108A}, 101's and Ar₁₀₁'s are the same as defined in the formula (40D).

Here, in the formula (42D-1), at least one of,
R_{101A}, and R_{103A} to R_{108A} which are hydrogen atoms,
hydrogen atoms possessed by R_{101A}, and R_{103A} to R_{108A} which are the substituents R,
hydrogen atoms possessed by L₁₀₁,
hydrogen atoms possessed by the substituent of L₁₀₁,
hydrogen atoms possessed by Ar₁₀₁
hydrogen atoms possessed by the substituent of Ar₁₀₁, and
hydrogen atoms bonding to the carbon atoms constituting the phenyl group in the formula (42D-1) is a deuterium atom.

At least one of, R_{101A}, and R_{103A} to R_{108A} which are hydrogen atoms in the formula (42D-2),
hydrogen atoms possessed by R_{101A}, and R_{103A} to R_{108A} which are the substituents R,
hydrogen atoms possessed by L₁₀₁,
hydrogen atoms possessed by the substituent of L₁₀₁,
hydrogen atoms possessed by Ar₁₀₁,
hydrogen atoms possessed by the substituent of Ar₁₀₁, and
hydrogen atoms bonding to the carbon atoms constituting the naphthyl group in the formula (42D-2) is a deuterium atom.

At least one of, R_{101A}, and R_{103A} to R_{108A} which are hydrogen atoms in the formula (42D-3),
hydrogen atoms possessed by R_{101A}, and R_{103A} to R_{108A} which are the substituents R,
hydrogen atoms possessed by L₁₀₁,
hydrogen atoms possessed by the substituent of L₁₀₁,
hydrogen atoms possessed by Ar₁₀₁,
hydrogen atoms possessed by the substituent of Ar₁₀₁ and
hydrogen atoms bonding to the carbon atoms constituting the naphthyl group in the formula (42D-3)
is a deuterium atom.

In one embodiment, the compounds represented by the formulas (42D-1) to (42D-3) are a compound represented by any one of the following formulas (43D-1) to (43D-3).

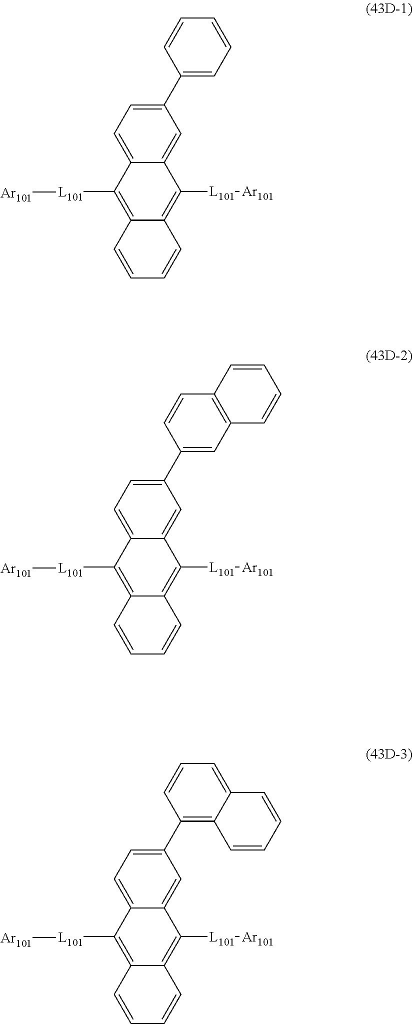

In the formula (43D-1) to (43D-3), L₁₀₁'s and Ar₁₀₁'s are the same as defined in the formula (40D).

Here, at least one of, hydrogen atoms bonding to the carbon atoms constituting the anthracene skeleton in the formula (43D-1),
hydrogen atoms possessed by L₁₀₁,
hydrogen atoms possessed by the substituent of L₁₀₁,
hydrogen atoms possessed by Ar₁₀₁,
hydrogen atoms possessed by the substituent of Ar₁₀₁, and
hydrogen atoms bonding to the carbon atoms constituting the phenyl group in the formula (43D-1) is a deuterium atom.

At least one of, hydrogen atoms bonding to the carbon atoms constituting the anthracene skeleton in the formula (43D-2),
hydrogen atoms possessed by L₁₀₁,
hydrogen atoms possessed by the substituent of L₁₀₁,
hydrogen atoms possessed by Ar₁₀₁,
hydrogen atoms possessed by the substituent of Ar₁₀₁, and
hydrogen atoms bonding to the carbon atoms constituting the naphthyl group in the formula (43D-2) is a deuterium atom.

At least one of, hydrogen atoms bonding to the carbon atoms constituting the anthracene skeleton in the formula (43D-3),
hydrogen atoms possessed by L₁₀₁,
hydrogen atoms possessed by the substituent of L₁₀₁,
hydrogen atoms possessed by Ar₁₀₁,
hydrogen atoms possessed by the substituent of Ar₁₀₁, and
hydrogen atoms bonding to the carbon atoms constituting the naphthyl group in the formula (43D-3)
is a deuterium atom.

In one embodiment, in the compound represented by the formula (20), at least one of Ar₁₀₁'s is a monovalent group having a structure represented by the following formula (50).

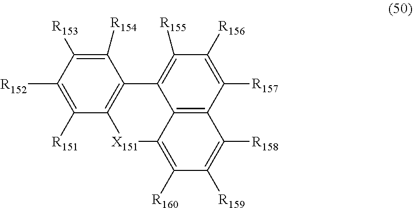

In the formula (50),
X1₅₁ is O, S or C(R₁₆₁)(R₁₆₂).
One of R₁₅₁ to R₁₆₀ is a single bond bonding with L₁₀₁.
One or more sets of, the adjacent two or more of R₁₅₁ to R₁₅₄ and the adjacent two or more of R₁₅₅ to R₁₆₀, which are not single bonds bonding with L₁₀₁, form a substituted or unsubstituted, saturated or unsaturated ring by bonding with each other, or do not form the substituted or unsubstituted, saturated or unsaturated ring.
R₁₆₁ and R₁₆₂ form a substituted or unsubstituted, saturated or unsaturated ring by bonding with each other, or do not form the substituted or unsubstituted, saturated or unsaturated ring.
R₁₆₁ and R₁₆₂ which do not form the substituted or unsubstituted, saturated or unsaturated ring, and R₁₅₁ to R₁₆₀ which are not single bonds bonding with L₁₀₁ and which do not form the substituted or unsubstituted, saturated or unsaturated ring are independently hydrogen atoms or substituents R.
The substituent R is the same as defined in the formula (10).
Ar₁₀₁ which is not the monovalent group having the structure represented by the formula (50) is
a substituted or unsubstituted aryl group having 6 to 50 ring carbon atoms, or
a substituted or unsubstituted divalent heterocyclic group having 5 to 50 ring atoms.

The position of the single bond to L₁₀₁ in the formula (50) is not particularly limited.

In one embodiment, one of R₁₅₁ to R₁₅₄ or one of R₁₅₅ to R₁ₗ₀ in the formula (50) is a single bond that binds to an L₁₀₁.

In one embodiment, Ar₁₀₁ is a monovalent group represented by the formula (50-R₁₅₂), (50-R₁₅₃), (50-R₁₅₄), (50-R₁₅₇) or (50-R₁₅₈).

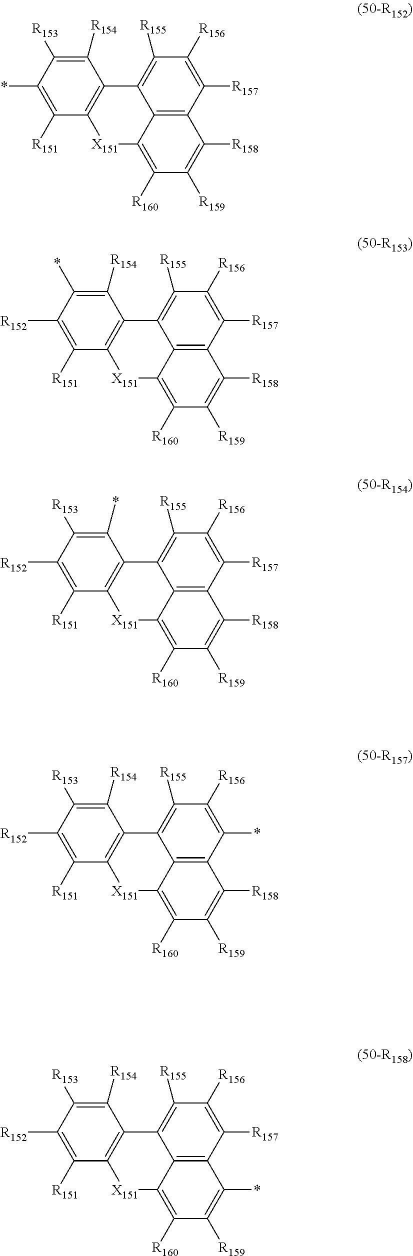

In the formulas (50-R₁₅₂), (50-R₁₅₃), (50-R₁₅₄), (50-R₁₅₇) and (50-R₁₅₈), X₁₅₁, and R₁₅₁ to R_{1c} are the same as defined in the formula (50).
* is bonded with L₁₀₁.

Specific examples of the compound represented by the formula (10) include the following compounds. The compound represented by the formula (10) is not limited to these specific examples. In the following specific examples, “D” represents a deuterium atom.

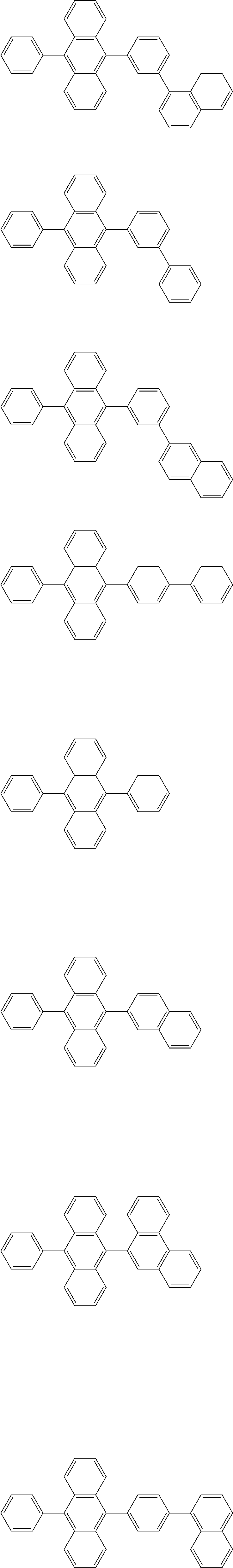

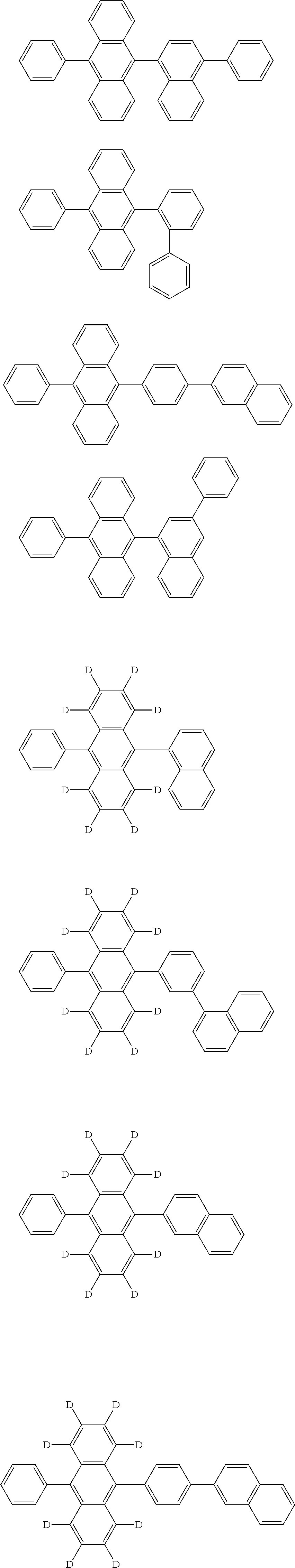

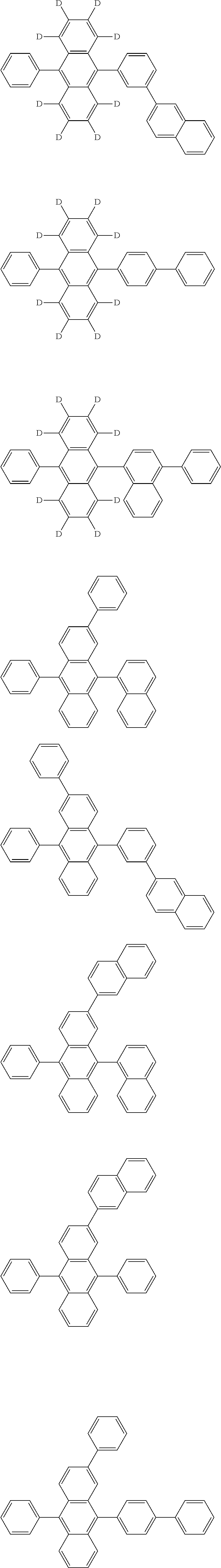

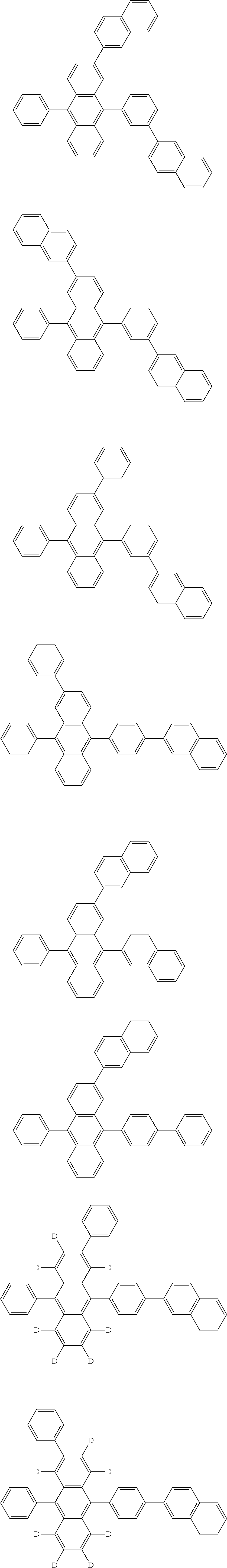

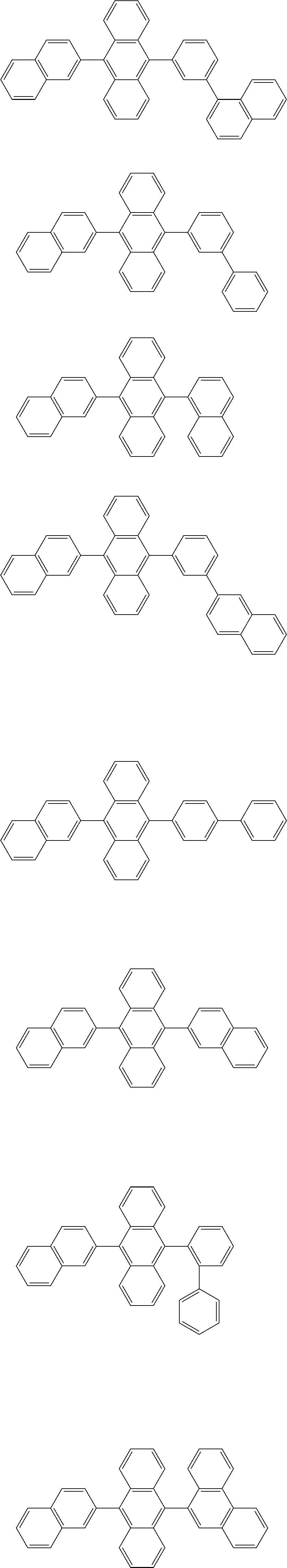

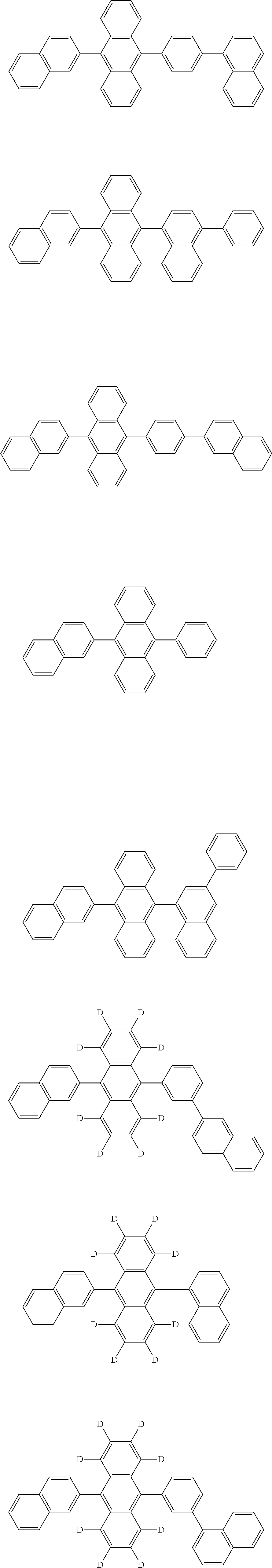

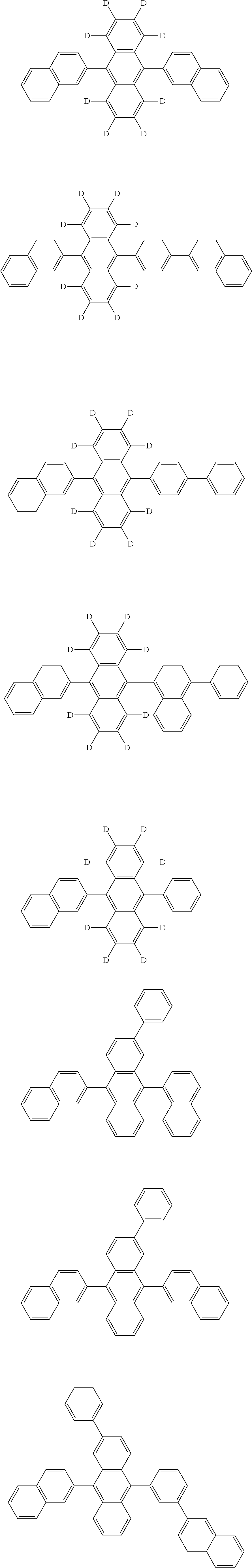

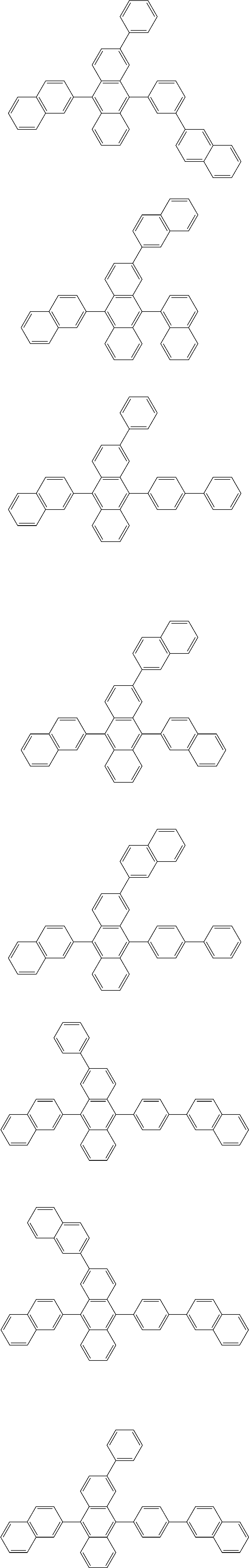

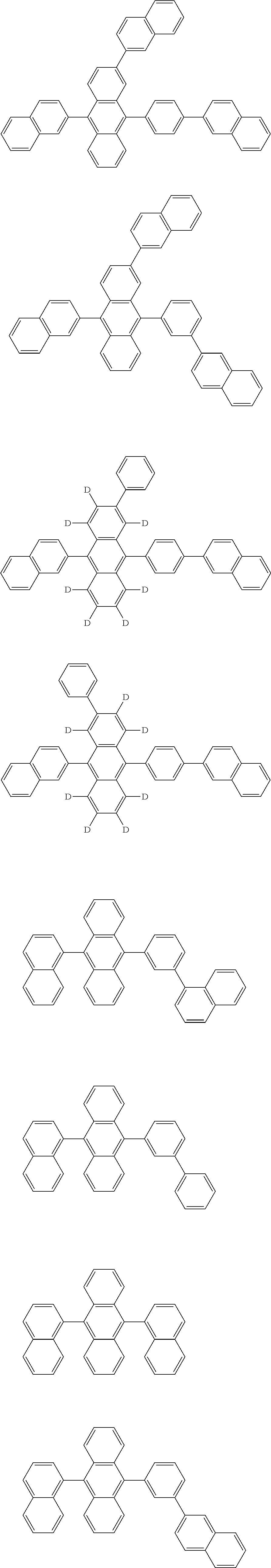

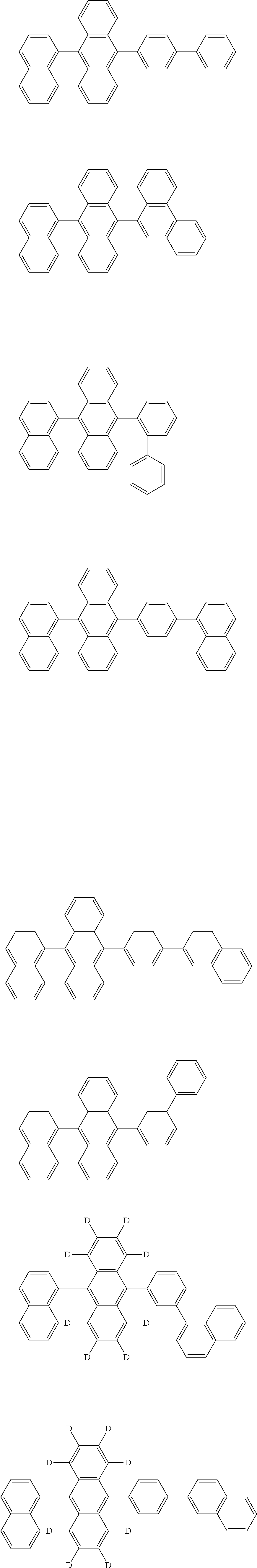

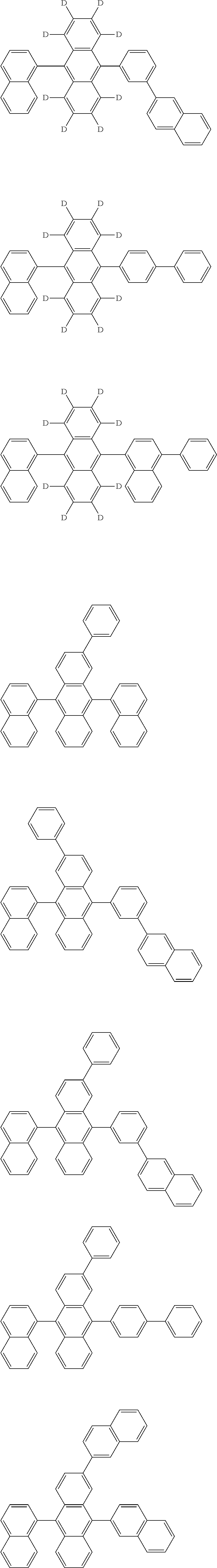

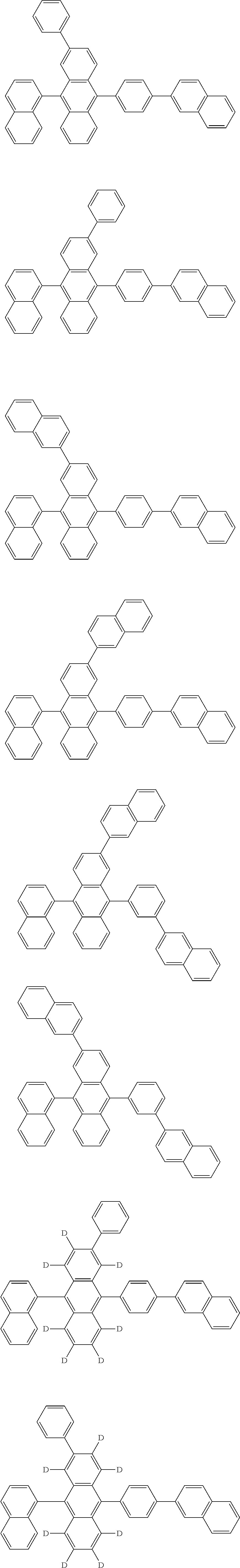

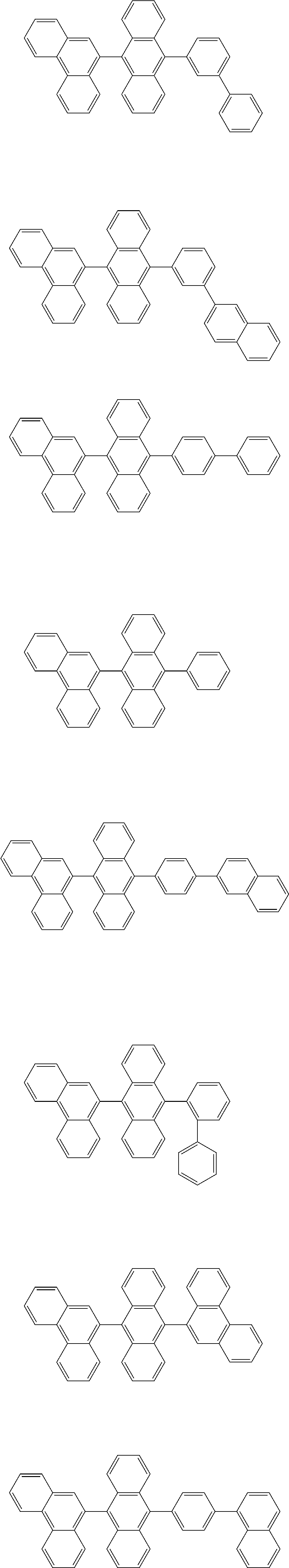

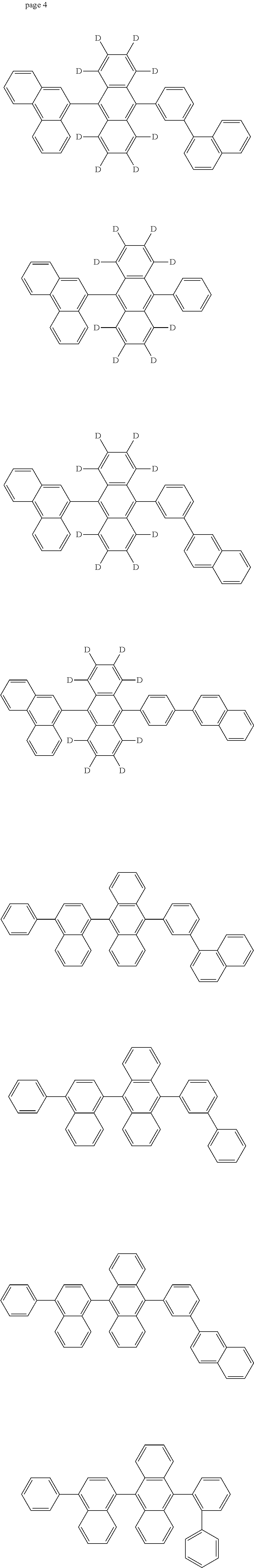

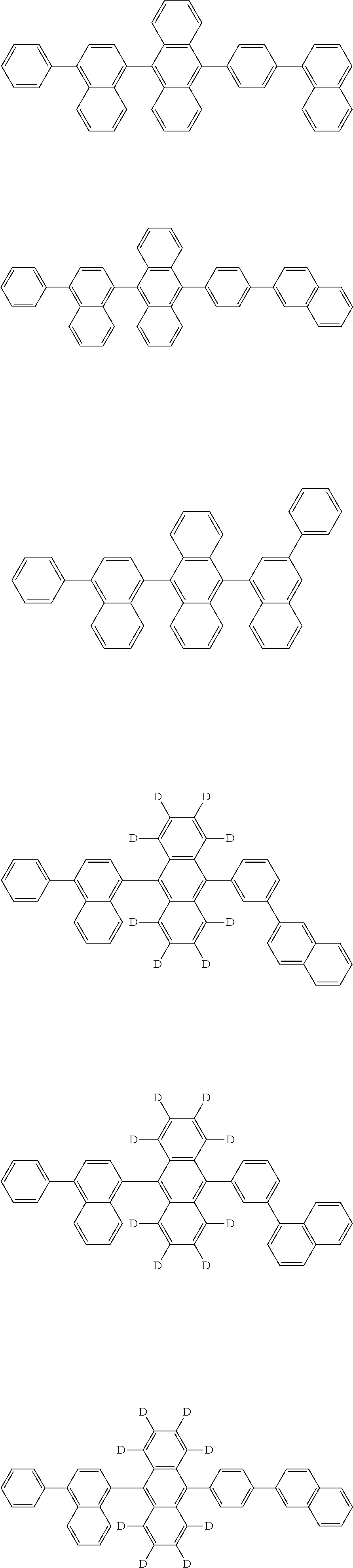

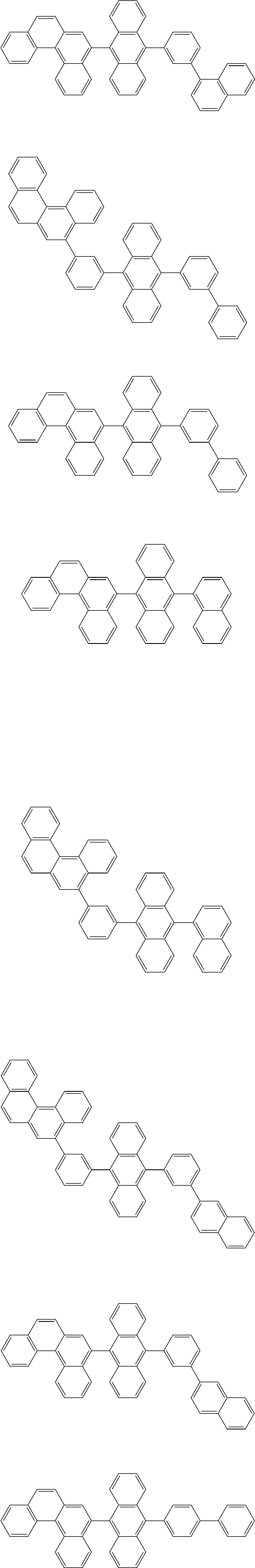

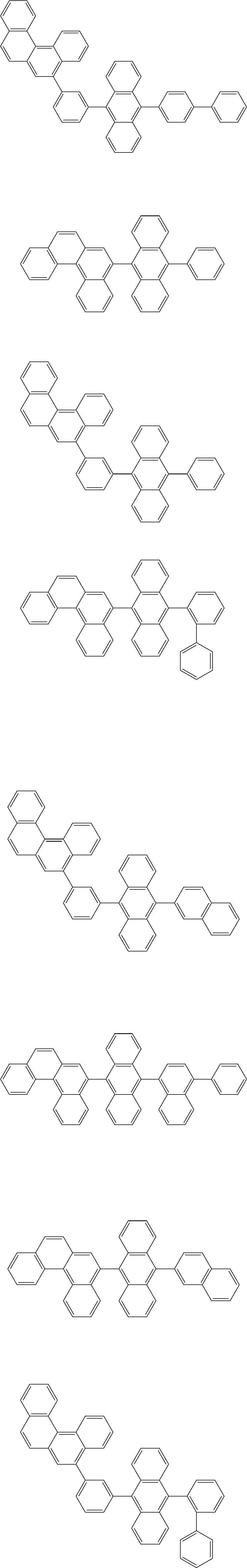

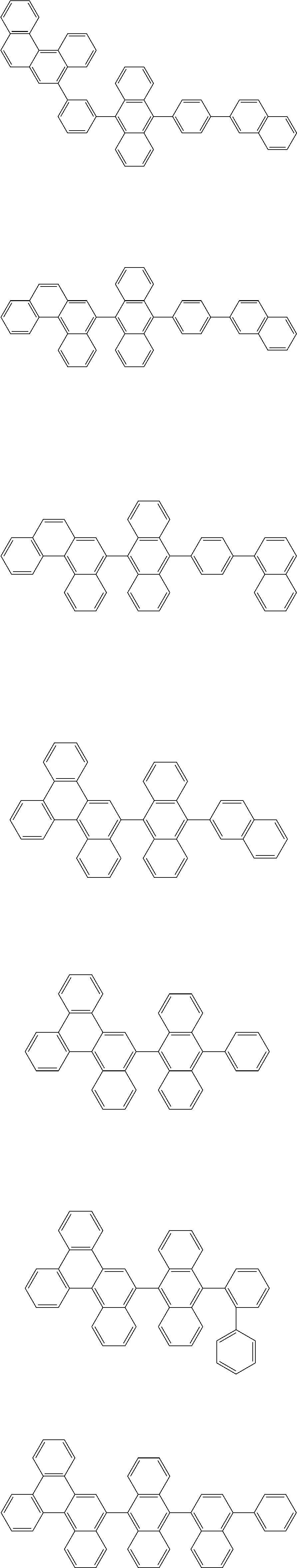

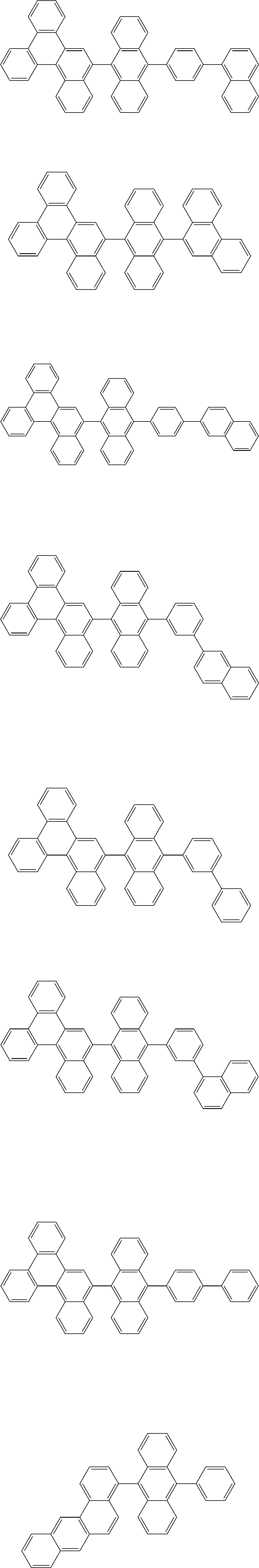

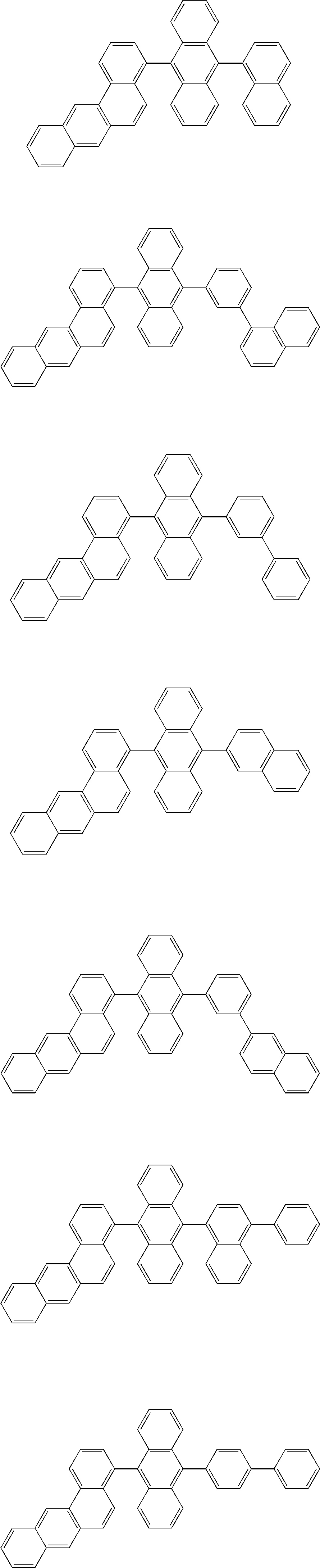

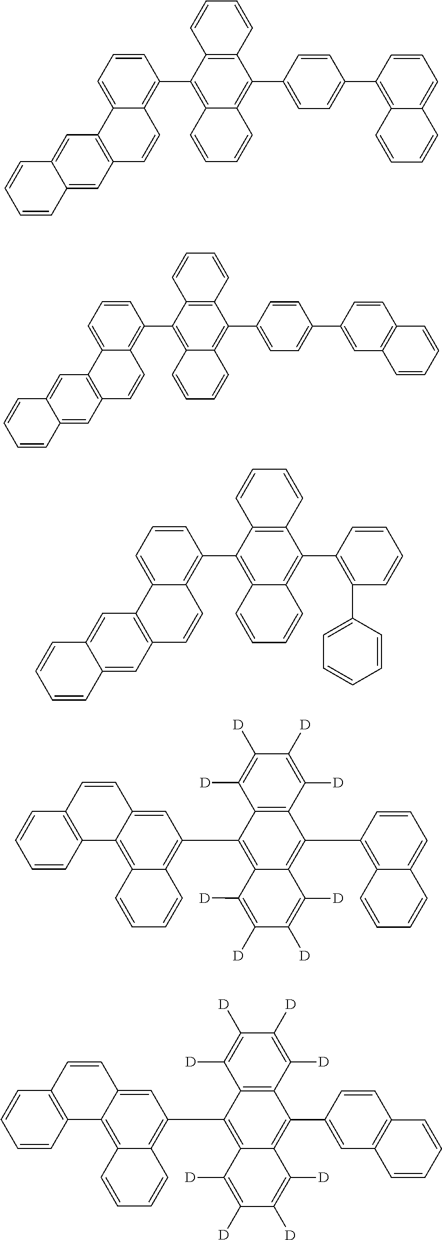

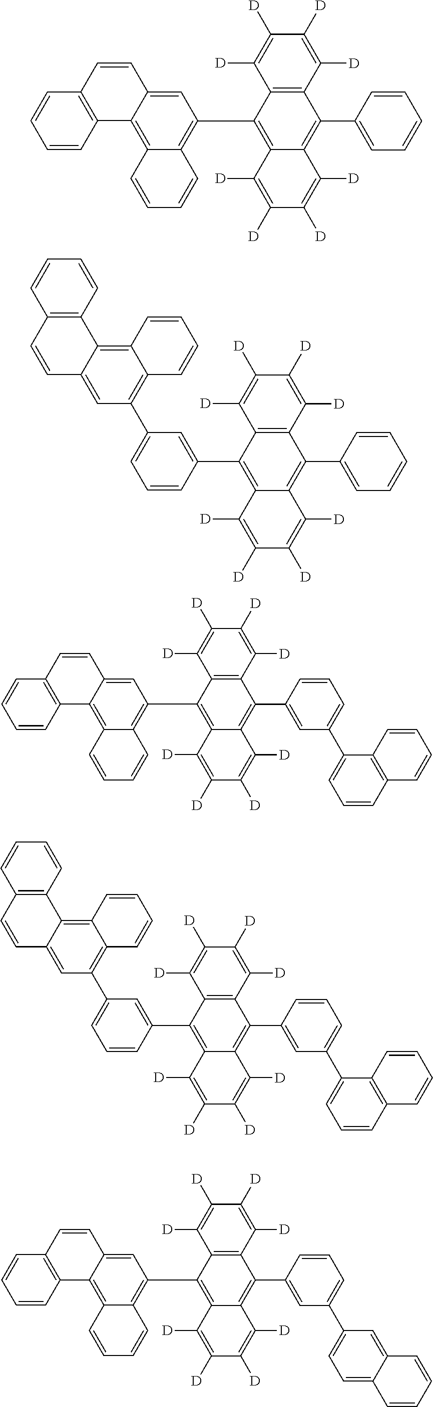

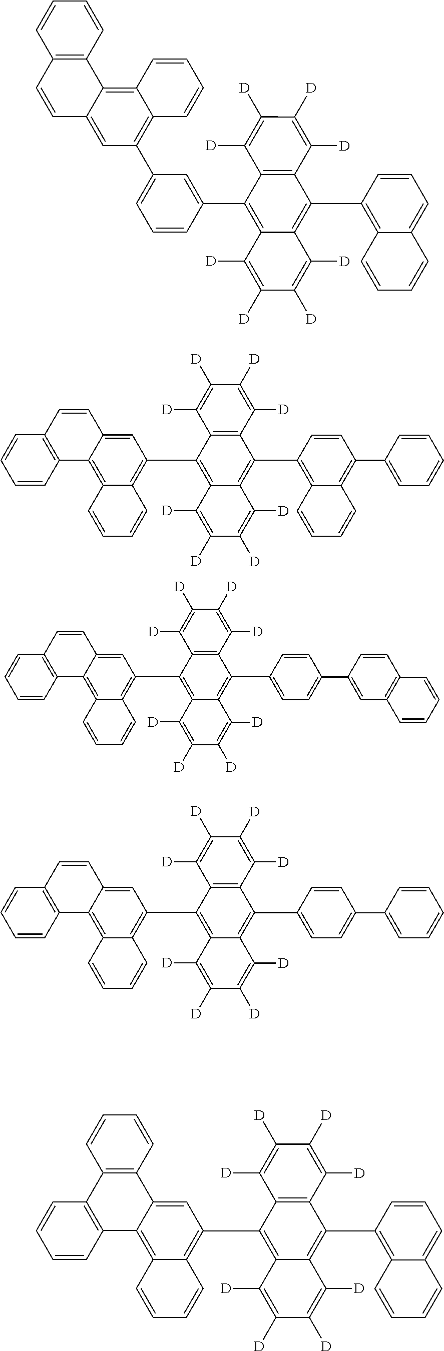

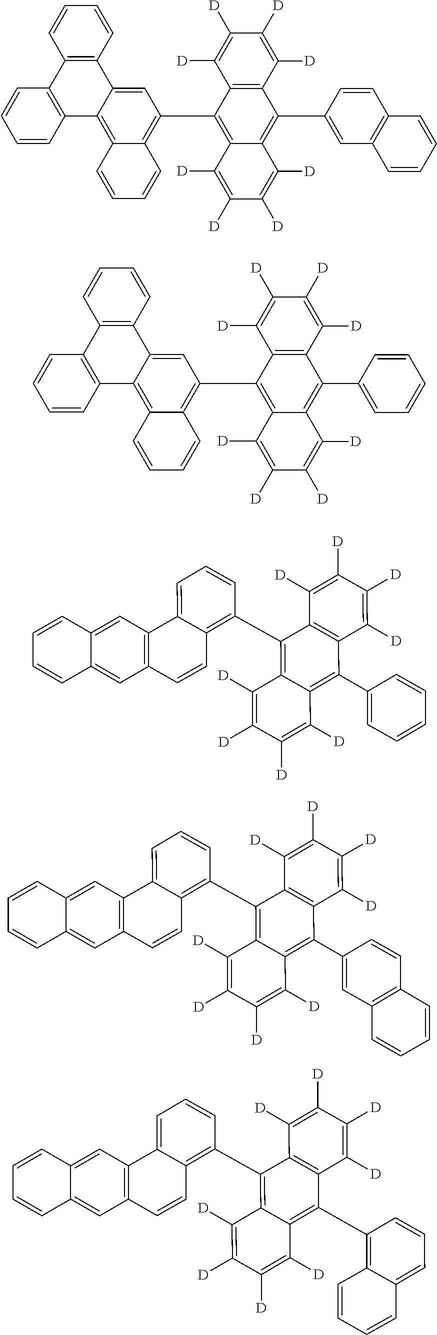

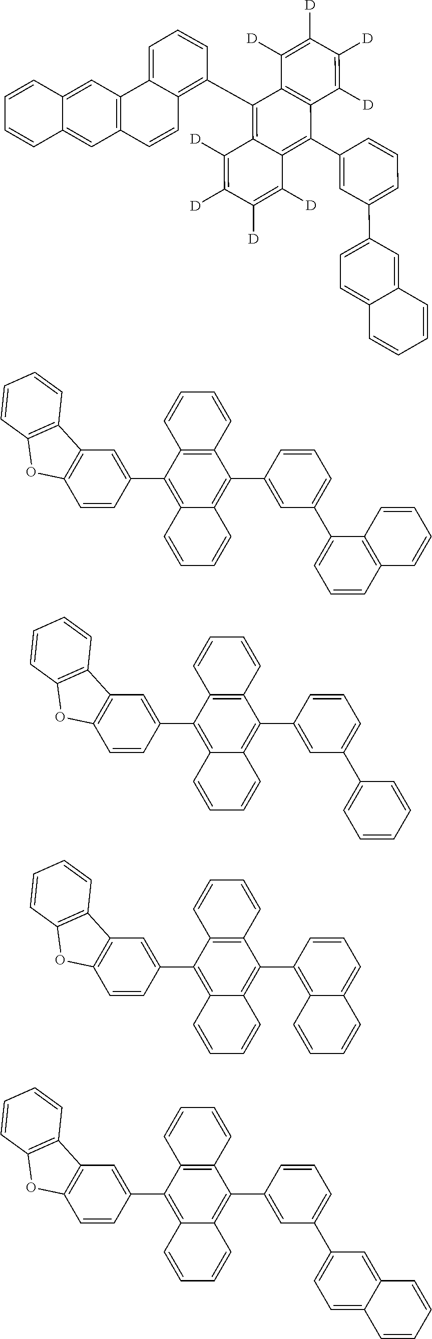 [SMILES: [2H]c1c([2H])c([2H])c2c(-c3cccc4c3ccc3cc5ccccc5cc34)c3c([2H])c([2H])c([2H])c([2H])c3c(-c3cccc(-c4ccc5ccccc5c4)c3)c2c1[2H] ; c1cc(-c2cccc3ccccc23)cc(-c2c3ccccc3c(-c3ccc4oc5ccccc5c4c3)c3ccccc23)c1 ; c1ccc(-c2cccc(-c3c4ccccc4c(-c4ccc5oc6ccccc6c5c4)c4ccccc34)c2)cc1 ; c1ccc2c(-c3c4ccccc4c(-c4ccc5oc6ccccc6c5c4)c4ccccc34)cccc2c1 ; c1cc(-c2ccc3ccccc3c2)cc(-c2c3ccccc3c(-c3ccc4oc5ccccc5c4c3)c3ccccc23)c1]

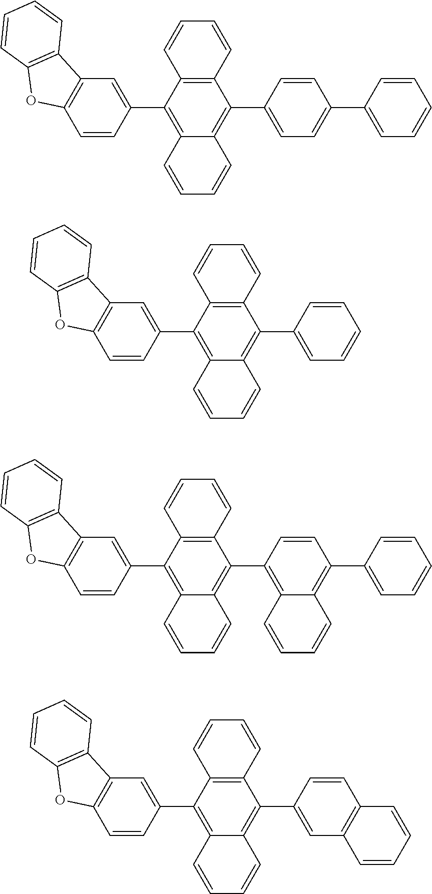 [SMILES: c1ccc(-c2ccc(-c3c4ccccc4c(-c4ccc5oc6ccccc6c5c4)c4ccccc34)cc2)cc1 ; c1ccc(-c2c3ccccc3c(-c3ccc4oc5ccccc5c4c3)c3ccccc23)cc1 ; c1ccc(-c2ccc(-c3c4ccccc4c(-c4ccc5oc6ccccc6c5c4)c4ccccc34)c3ccccc23)cc1 ; c1ccc2cc(-c3c4ccccc4c(-c4ccc5oc6ccccc6c5c4)c4ccccc34)ccc2c1]

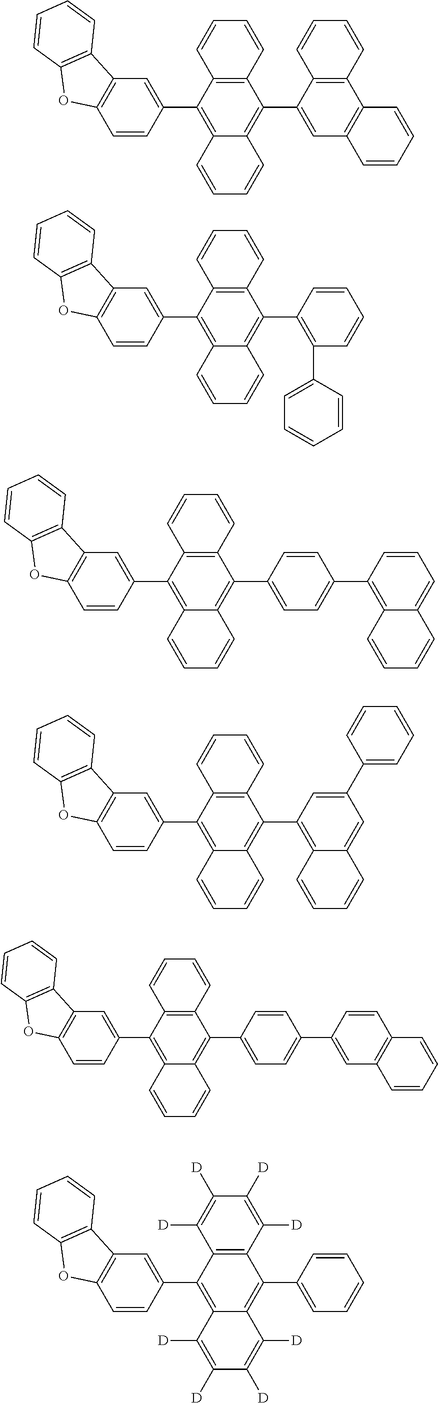

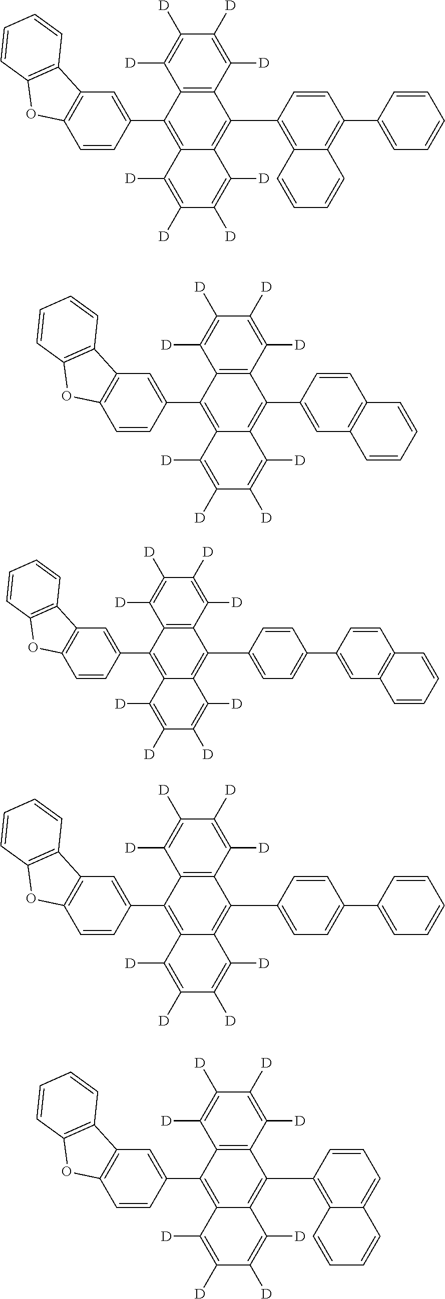

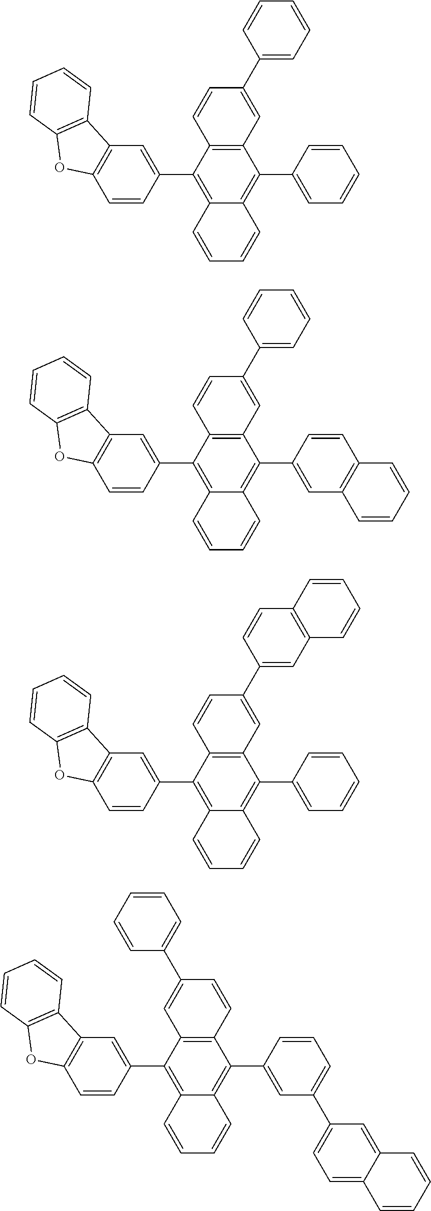

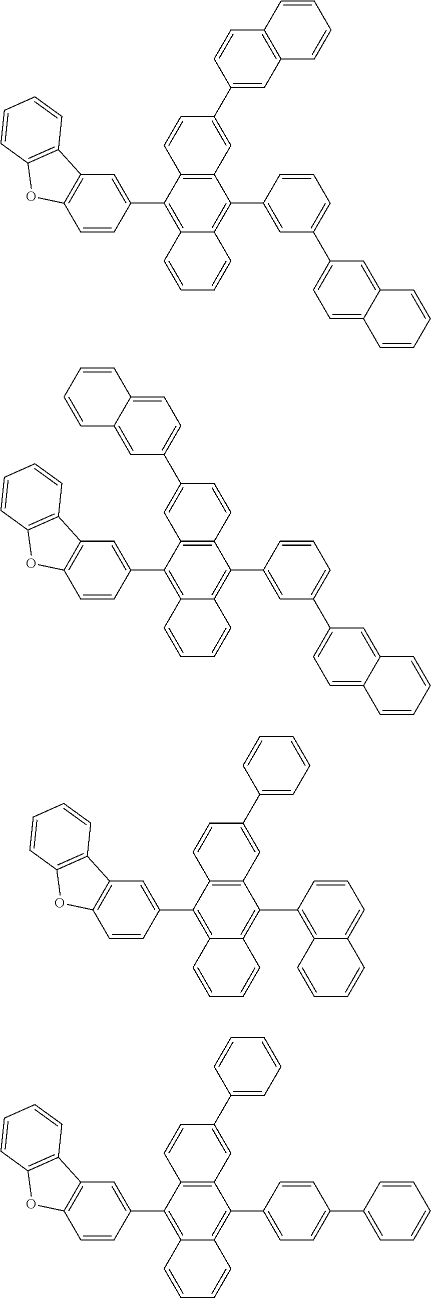

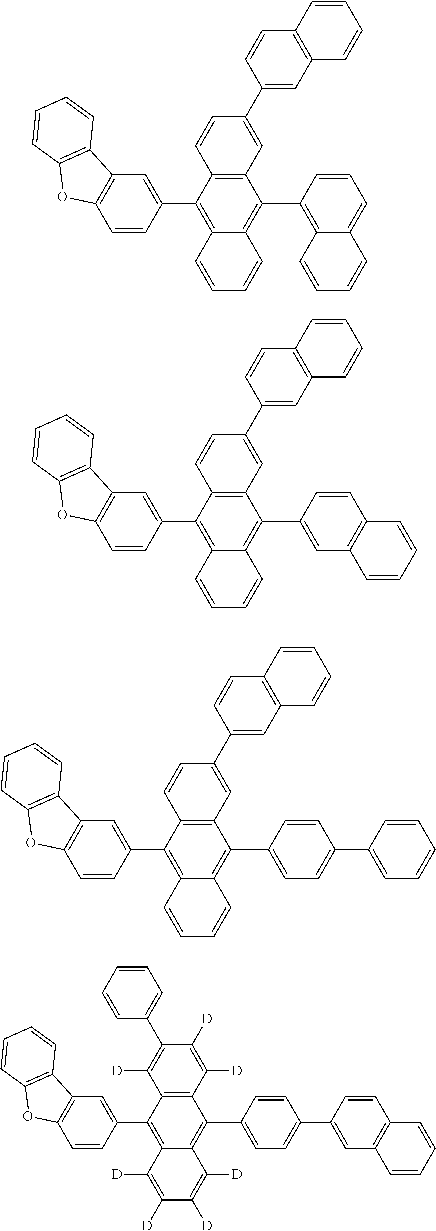

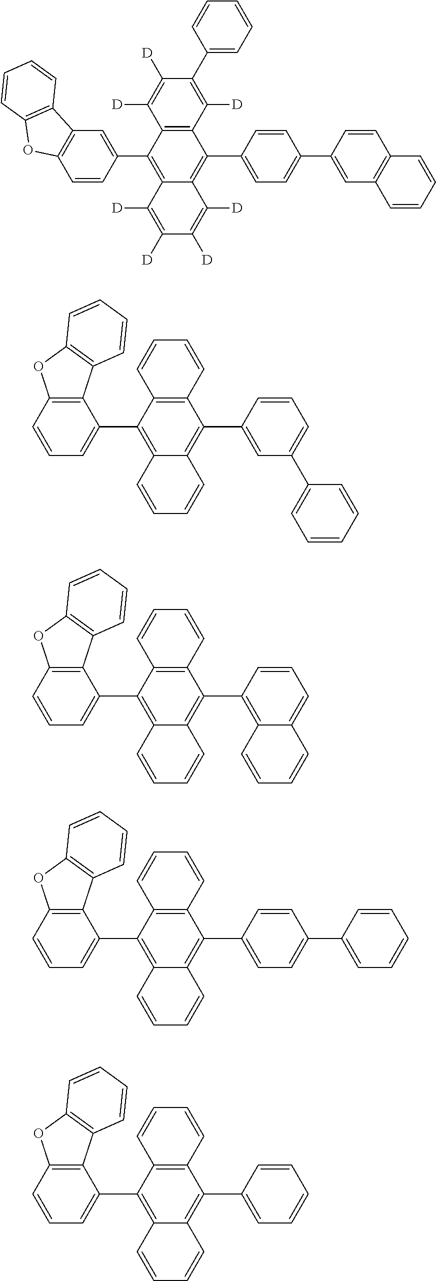 [SMILES: [2H]c1c([2H])c([2H])c2c(-c3ccc(-c4ccc5ccccc5c4)cc3)c3c([2H])c(-c4ccccc4)c([2H])c([2H])c3c(-c3ccc4oc5ccccc5c4c3)c2c1[2H] ; c1ccc(-c2cccc(-c3c4ccccc4c(-c4cccc5oc6ccccc6c45)c4ccccc34)c2)cc1 ; c1ccc2c(-c3c4ccccc4c(-c4cccc5oc6ccccc6c45)c4ccccc34)cccc2c1 ; c1ccc(-c2ccc(-c3c4ccccc4c(-c4cccc5oc6ccccc6c45)c4ccccc34)cc2)cc1 ; c1ccc(-c2c3ccccc3c(-c3cccc4oc5ccccc5c34)c3ccccc23)cc1]

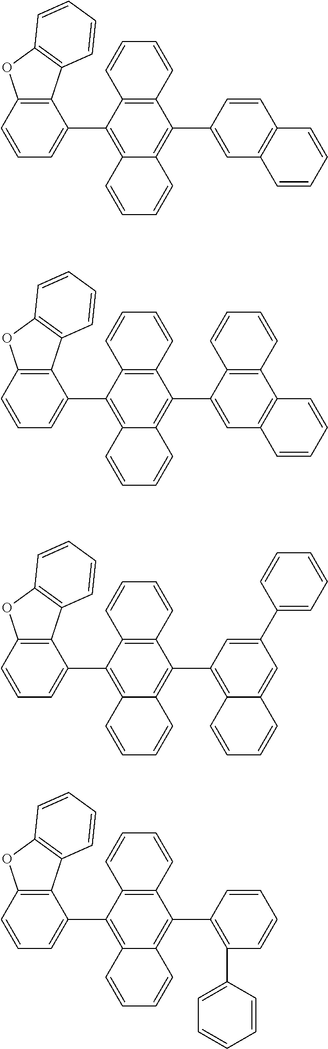 [SMILES: c1ccc2cc(-c3c4ccccc4c(-c4cccc5oc6ccccc6c45)c4ccccc34)ccc2c1 ; c1ccc2c(c1)cc(-c1c3ccccc3c(-c3cccc4oc5ccccc5c34)c3ccccc13)c1ccccc12 ; c1ccc(-c2cc(-c3c4ccccc4c(-c4cccc5oc6ccccc6c45)c4ccccc34)c3ccccc3c2)cc1 ; c1ccc(-c2ccccc2-c2c3ccccc3c(-c3cccc4oc5ccccc5c34)c3ccccc23)cc1]

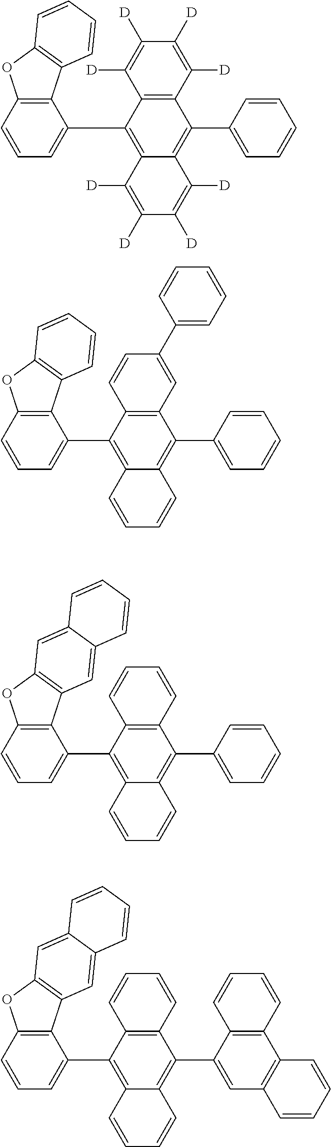

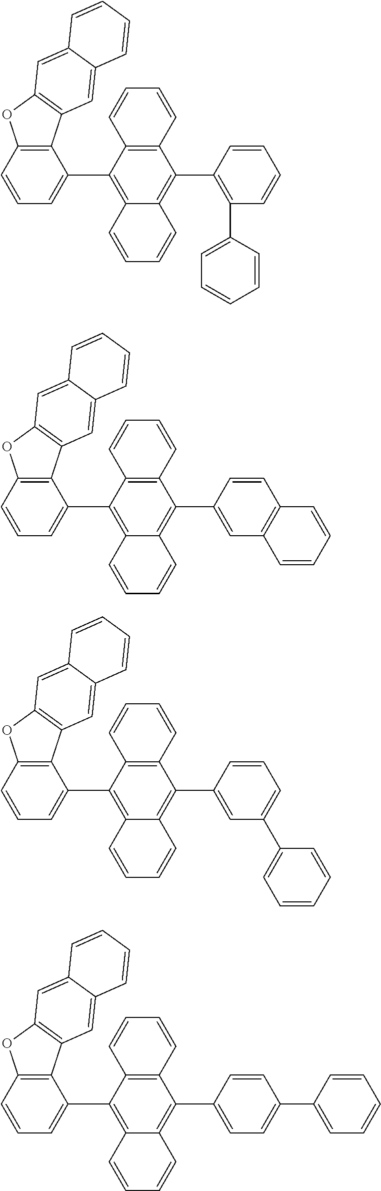

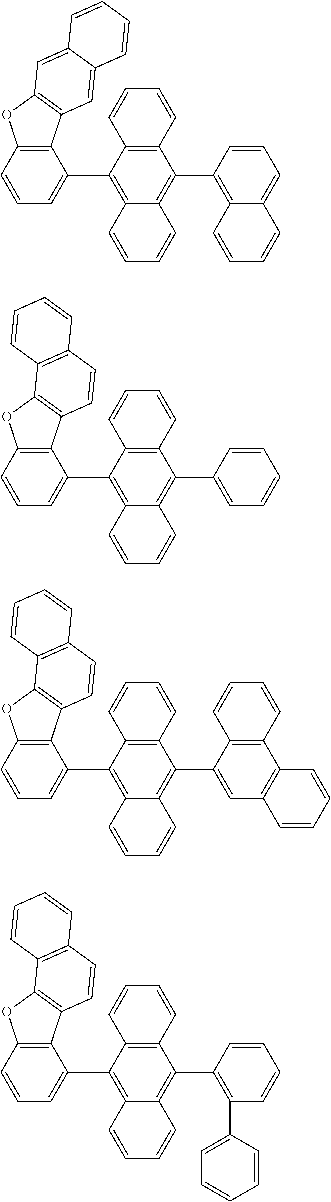 [SMILES: c1ccc2cc3c(cc2c1)oc1cccc(-c2c4ccccc4c(-c4cccc5ccccc45)c4ccccc24)c13 ; c1ccc(-c2c3ccccc3c(-c3cccc4oc5c6ccccc6ccc5c34)c3ccccc23)cc1 ; c1ccc2c(c1)cc(-c1c3ccccc3c(-c3cccc4oc5c6ccccc6ccc5c34)c3ccccc13)c1ccccc12 ; c1ccc(-c2ccccc2-c2c3ccccc3c(-c3cccc4oc5c6ccccc6ccc5c34)c3ccccc23)cc1]

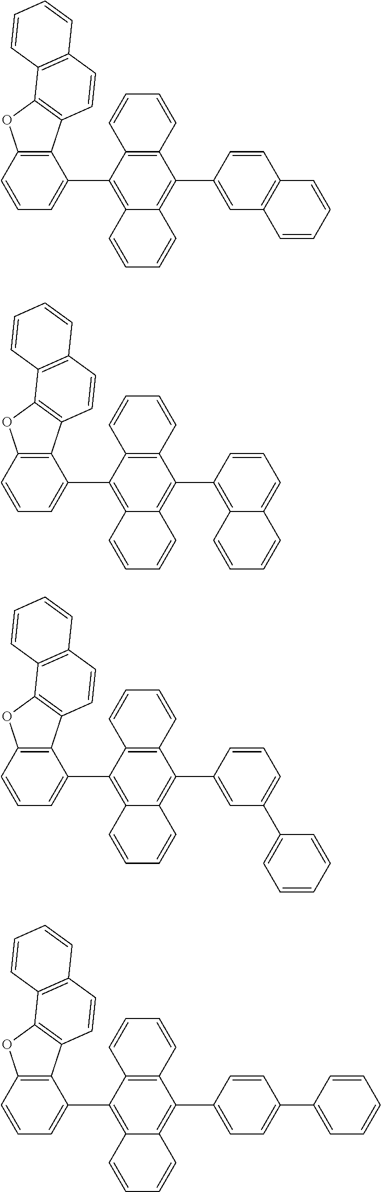 [SMILES: c1ccc2cc(-c3c4ccccc4c(-c4cccc5oc6c7ccccc7ccc6c45)c4ccccc34)ccc2c1 ; c1ccc2c(-c3c4ccccc4c(-c4cccc5oc6c7ccccc7ccc6c45)c4ccccc34)cccc2c1 ; c1ccc(-c2cccc(-c3c4ccccc4c(-c4cccc5oc6c7ccccc7ccc6c45)c4ccccc34)c2)cc1 ; c1ccc(-c2ccc(-c3c4ccccc4c(-c4cccc5oc6c7ccccc7ccc6c45)c4ccccc34)cc2)cc1]

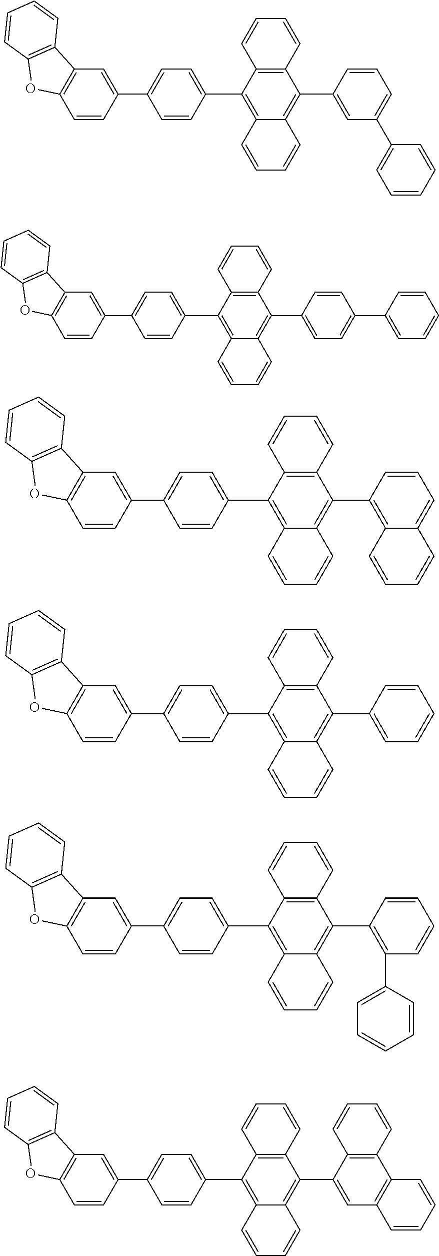

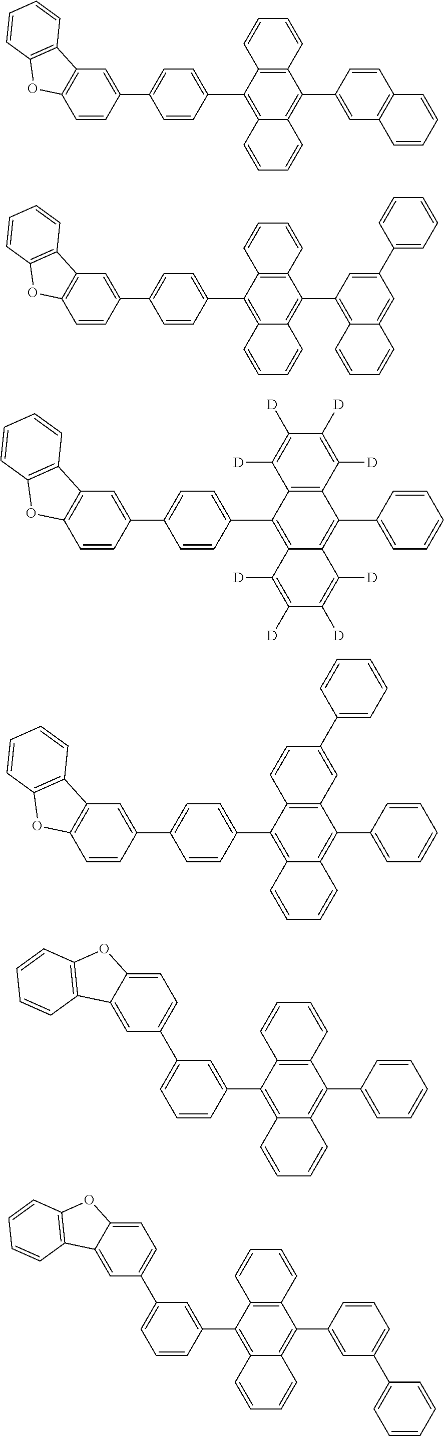

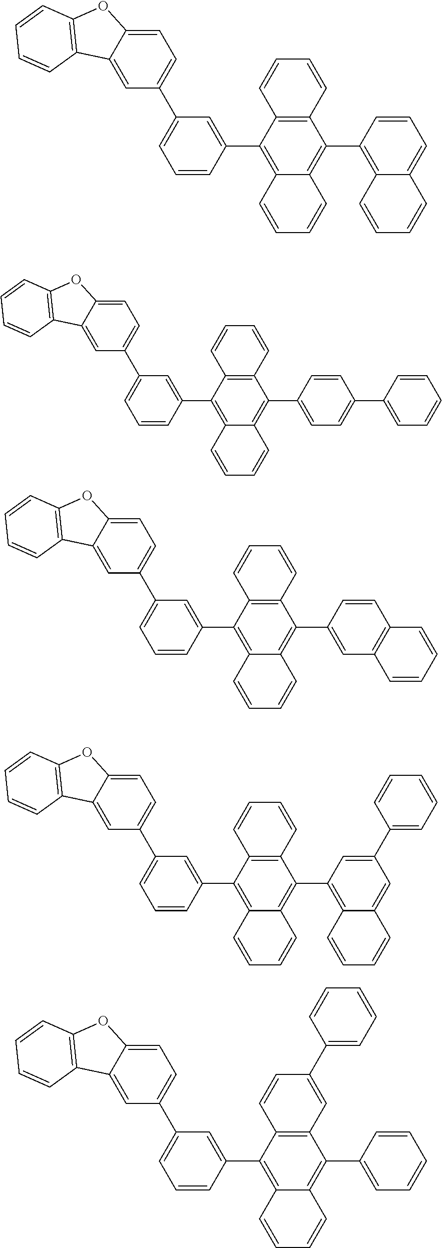

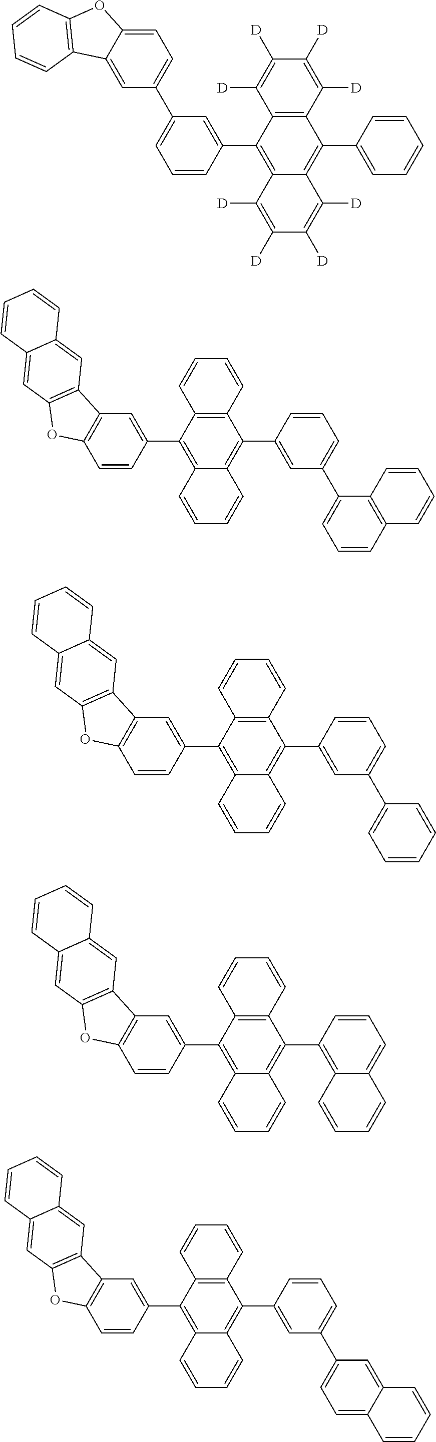 [SMILES: [2H]c1c([2H])c([2H])c2c(-c3cccc(-c4ccc5oc6ccccc6c5c4)c3)c3c([2H])c([2H])c([2H])c([2H])c3c(-c3ccccc3)c2c1[2H] ; c1cc(-c2cccc3ccccc23)cc(-c2c3ccccc3c(-c3ccc4oc5cc6ccccc6cc5c4c3)c3ccccc23)c1 ; c1ccc(-c2cccc(-c3c4ccccc4c(-c4ccc5oc6cc7ccccc7cc6c5c4)c4ccccc34)c2)cc1 ; c1ccc2cc3c(cc2c1)oc1ccc(-c2c4ccccc4c(-c4cccc5ccccc45)c4ccccc24)cc13 ; c1cc(-c2ccc3ccccc3c2)cc(-c2c3ccccc3c(-c3ccc4oc5cc6ccccc6cc5c4c3)c3ccccc23)c1]

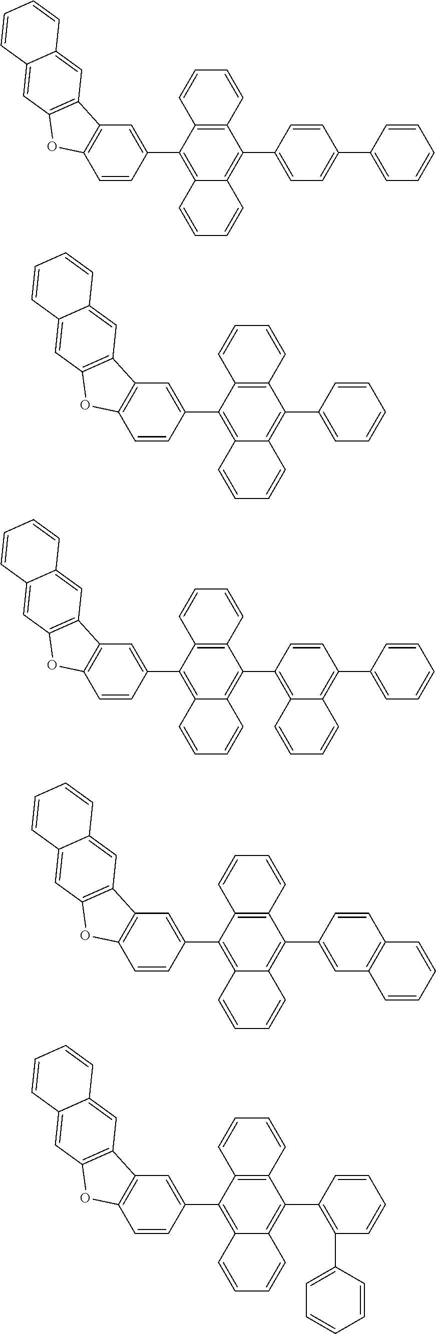 [SMILES: c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc(-c3c4ccccc4c(-c4ccc5oc6cc7ccccc7cc6c5c4)c4ccccc34)c3ccccc23)cc1 ; c1ccc2cc(-c3c4ccccc4c(-c4ccc5oc6cc7ccccc7cc6c5c4)c4ccccc34)ccc2c1 ; c1ccc(-c2ccccc2-c2c3ccccc3c(-c3ccc4oc5cc6ccccc6cc5c4c3)c3ccccc23)cc1]

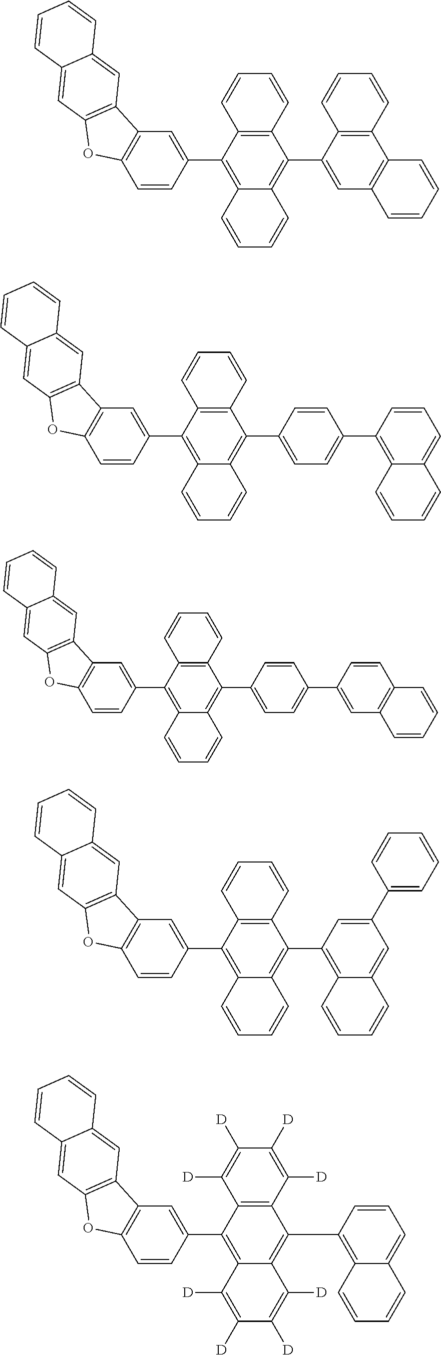 [SMILES: c1ccc2cc3c(cc2c1)oc1ccc(-c2c4ccccc4c(-c4cc5ccccc5c5ccccc45)c4ccccc24)cc13 ; c1ccc2cc3c(cc2c1)oc1ccc(-c2c4ccccc4c(-c4ccc(-c5cccc6ccccc56)cc4)c4ccccc24)cc13 ; c1ccc2cc(-c3ccc(-c4c5ccccc5c(-c5ccc6oc7cc8ccccc8cc7c6c5)c5ccccc45)cc3)ccc2c1 ; c1ccc(-c2cc(-c3c4ccccc4c(-c4ccc5oc6cc7ccccc7cc6c5c4)c4ccccc34)c3ccccc3c2)cc1 ; [2H]c1c([2H])c([2H])c2c(-c3cccc4ccccc34)c3c([2H])c([2H])c([2H])c([2H])c3c(-c3ccc4oc5cc6ccccc6cc5c4c3)c2c1[2H]]

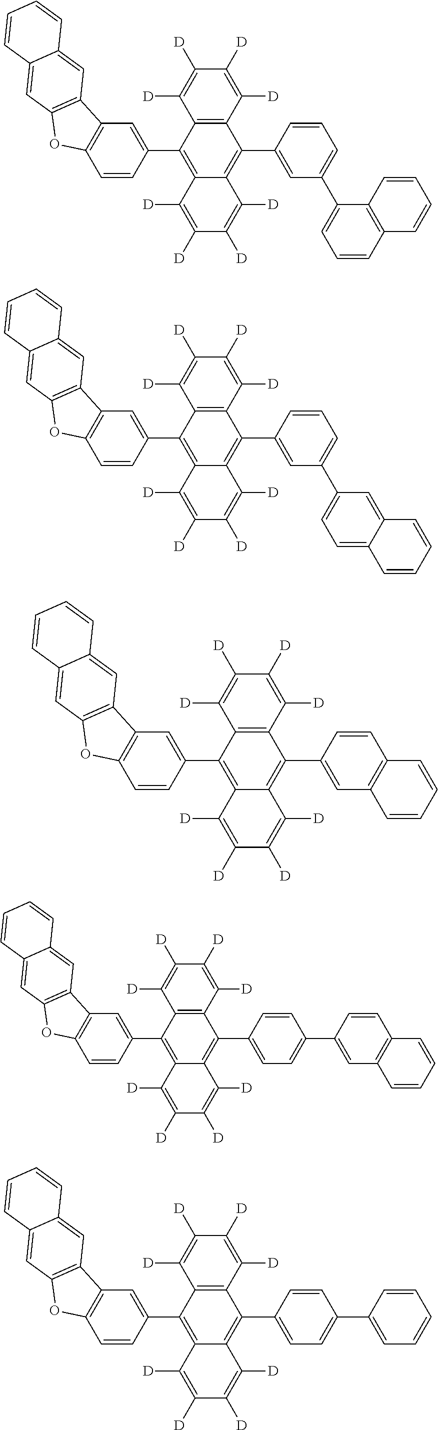

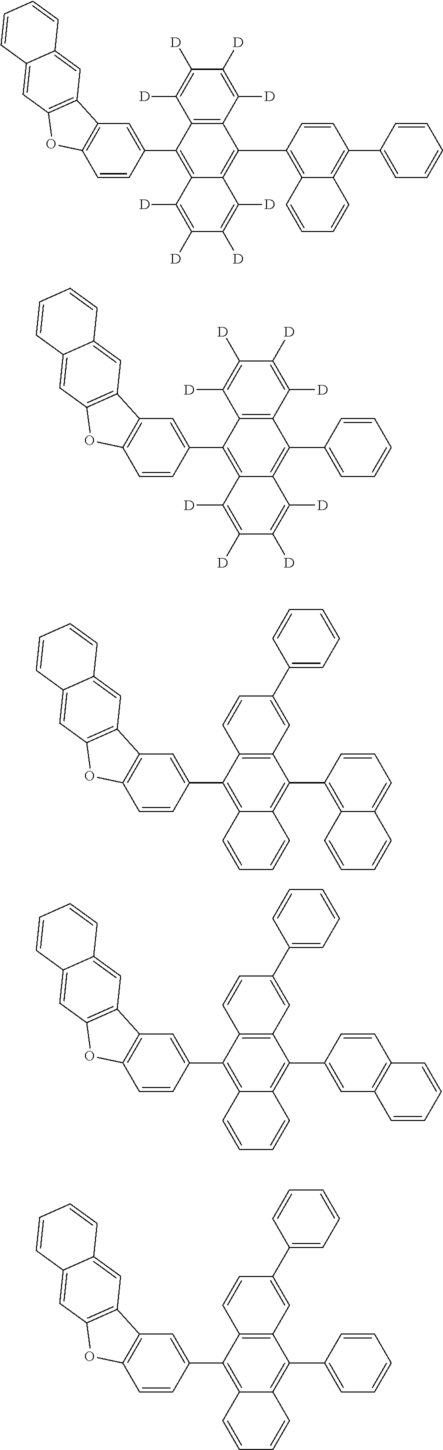

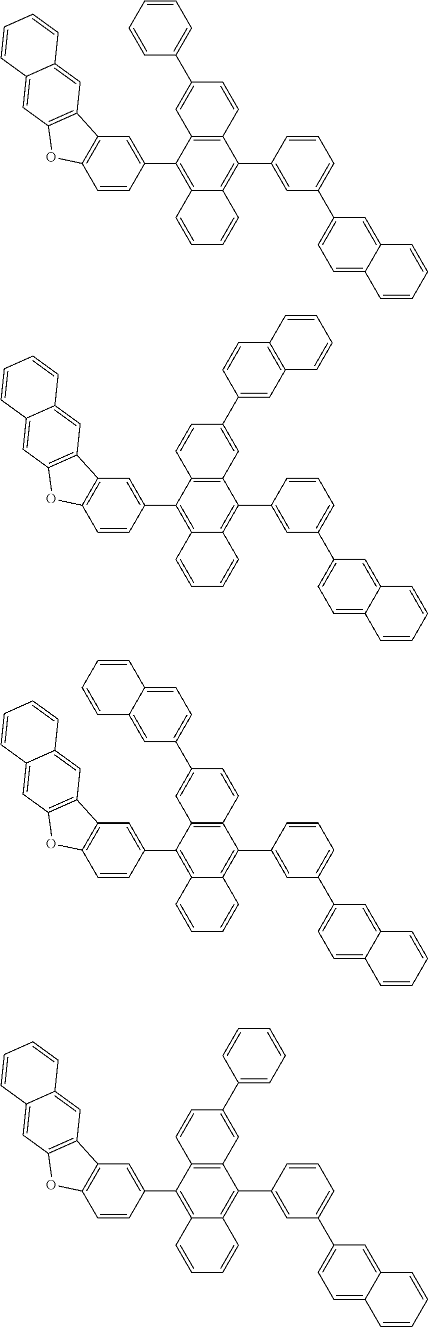 [SMILES: c1ccc(-c2ccc3c(-c4cccc(-c5ccc6ccccc6c5)c4)c4ccccc4c(-c4ccc5oc6cc7ccccc7cc6c5c4)c3c2)cc1 ; c1cc(-c2ccc3ccccc3c2)cc(-c2c3ccccc3c(-c3ccc4oc5cc6ccccc6cc5c4c3)c3ccc(-c4ccc5ccccc5c4)cc23)c1 ; c1cc(-c2ccc3ccccc3c2)cc(-c2c3ccccc3c(-c3ccc4oc5cc6ccccc6cc5c4c3)c3cc(-c4ccc5ccccc5c4)ccc23)c1 ; c1ccc(-c2ccc3c(-c4ccc5oc6cc7ccccc7cc6c5c4)c4ccccc4c(-c4cccc(-c5ccc6ccccc6c5)c4)c3c2)cc1]

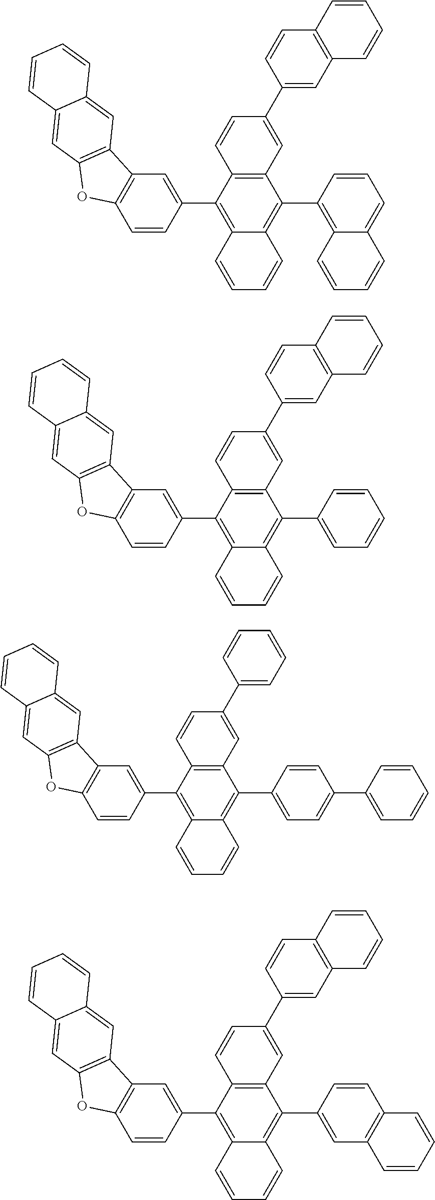

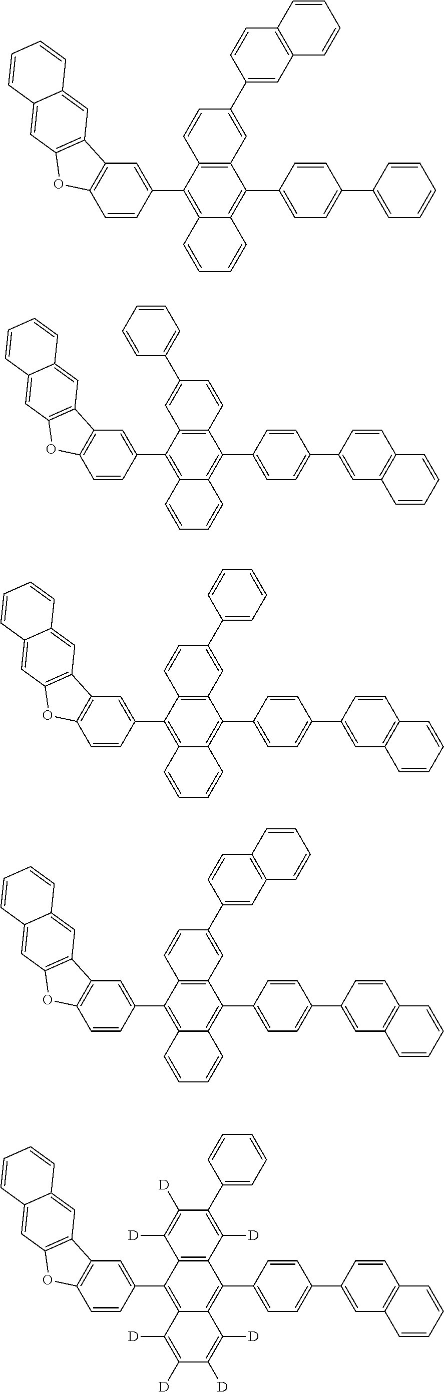 [SMILES: c1ccc(-c2ccc(-c3c4ccccc4c(-c4ccc5oc6cc7ccccc7cc6c5c4)c4ccc(-c5ccc6ccccc6c5)cc34)cc2)cc1 ; c1ccc(-c2ccc3c(-c4ccc(-c5ccc6ccccc6c5)cc4)c4ccccc4c(-c4ccc5oc6cc7ccccc7cc6c5c4)c3c2)cc1 ; c1ccc(-c2ccc3c(-c4ccc5oc6cc7ccccc7cc6c5c4)c4ccccc4c(-c4ccc(-c5ccc6ccccc6c5)cc4)c3c2)cc1 ; c1ccc2cc(-c3ccc(-c4c5ccccc5c(-c5ccc6oc7cc8ccccc8cc7c6c5)c5ccc(-c6ccc7ccccc7c6)cc45)cc3)ccc2c1 ; [2H]c1c([2H])c([2H])c2c(-c3ccc(-c4ccc5ccccc5c4)cc3)c3c([2H])c(-c4ccccc4)c([2H])c([2H])c3c(-c3ccc4oc5cc6ccccc6cc5c4c3)c2c1[2H]]

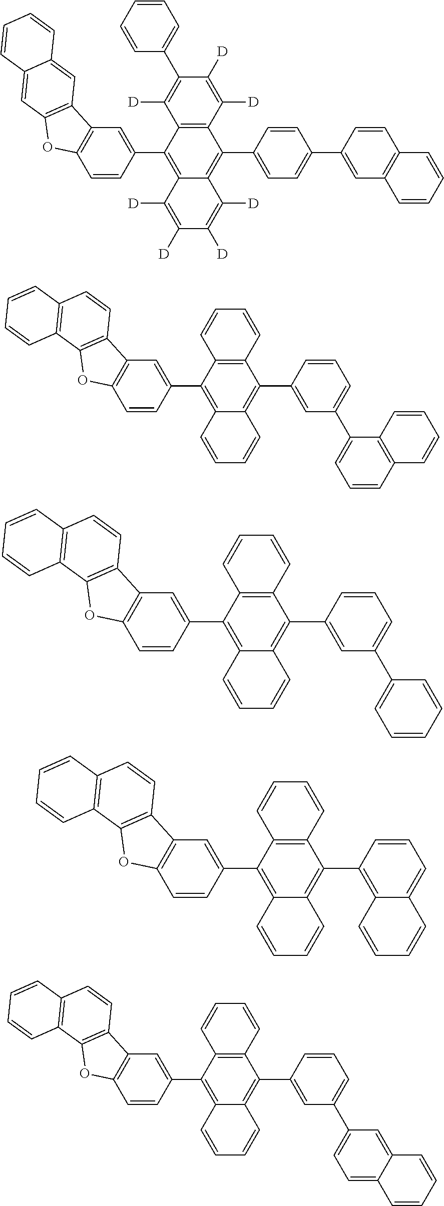 [SMILES: [2H]c1c([2H])c([2H])c2c(-c3ccc4oc5cc6ccccc6cc5c4c3)c3c([2H])c(-c4ccccc4)c([2H])c([2H])c3c(-c3ccc(-c4ccc5ccccc5c4)cc3)c2c1[2H] ; c1cc(-c2cccc3ccccc23)cc(-c2c3ccccc3c(-c3ccc4oc5c6ccccc6ccc5c4c3)c3ccccc23)c1 ; c1ccc(-c2cccc(-c3c4ccccc4c(-c4ccc5oc6c7ccccc7ccc6c5c4)c4ccccc34)c2)cc1 ; c1ccc2c(-c3c4ccccc4c(-c4ccc5oc6c7ccccc7ccc6c5c4)c4ccccc34)cccc2c1 ; c1cc(-c2ccc3ccccc3c2)cc(-c2c3ccccc3c(-c3ccc4oc5c6ccccc6ccc5c4c3)c3ccccc23)c1]

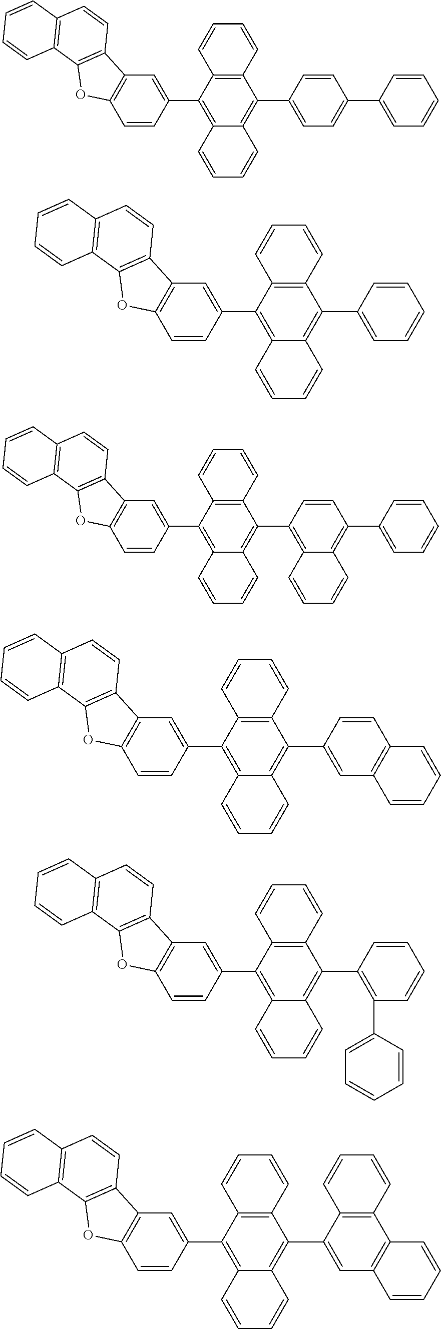 [SMILES: c1ccc(-c2ccc(-c3c4ccccc4c(-c4ccc5oc6c7ccccc7ccc6c5c4)c4ccccc34)cc2)cc1 ; c1ccc(-c2c3ccccc3c(-c3ccc4oc5c6ccccc6ccc5c4c3)c3ccccc23)cc1 ; c1ccc(-c2ccc(-c3c4ccccc4c(-c4ccc5oc6c7ccccc7ccc6c5c4)c4ccccc34)c3ccccc23)cc1 ; c1ccc2cc(-c3c4ccccc4c(-c4ccc5oc6c7ccccc7ccc6c5c4)c4ccccc34)ccc2c1 ; c1ccc(-c2ccccc2-c2c3ccccc3c(-c3ccc4oc5c6ccccc6ccc5c4c3)c3ccccc23)cc1 ; c1ccc2c(c1)cc(-c1c3ccccc3c(-c3ccc4oc5c6ccccc6ccc5c4c3)c3ccccc13)c1ccccc12]

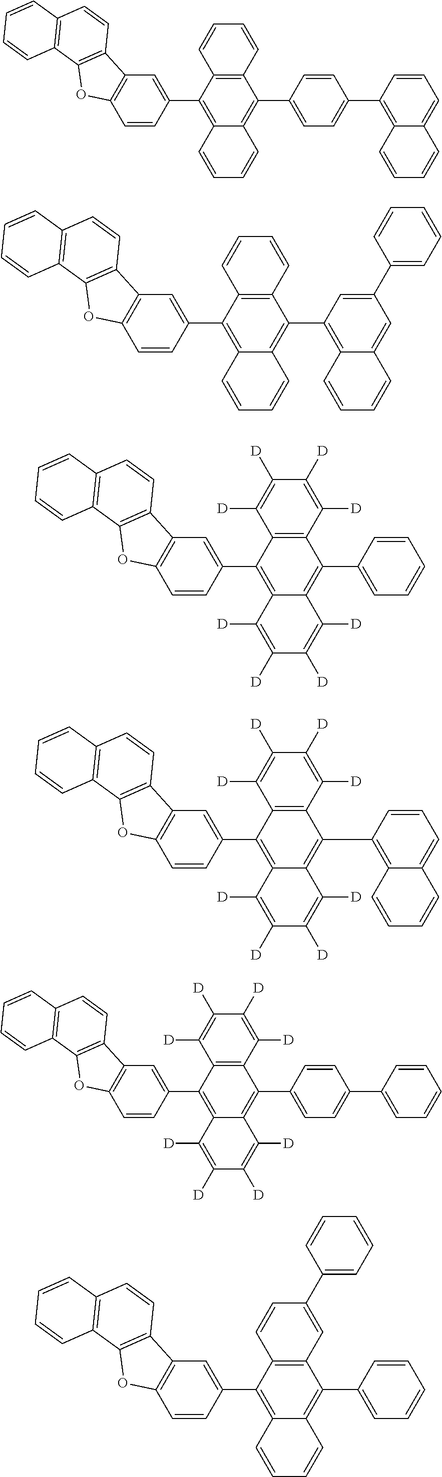

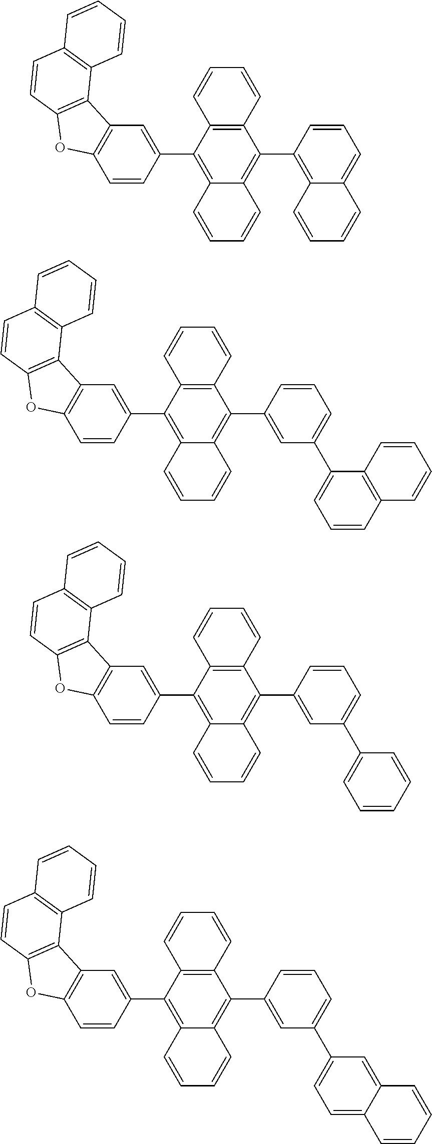 [SMILES: c1ccc2c(-c3c4ccccc4c(-c4ccc5oc6ccc7ccccc7c6c5c4)c4ccccc34)cccc2c1 ; c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1 ; c1cc(-c2ccc3ccccc3c2)cc(-c2c3ccccc3c(-c3ccc4oc5ccc6ccccc6c5c4c3)c3ccccc23)c1]

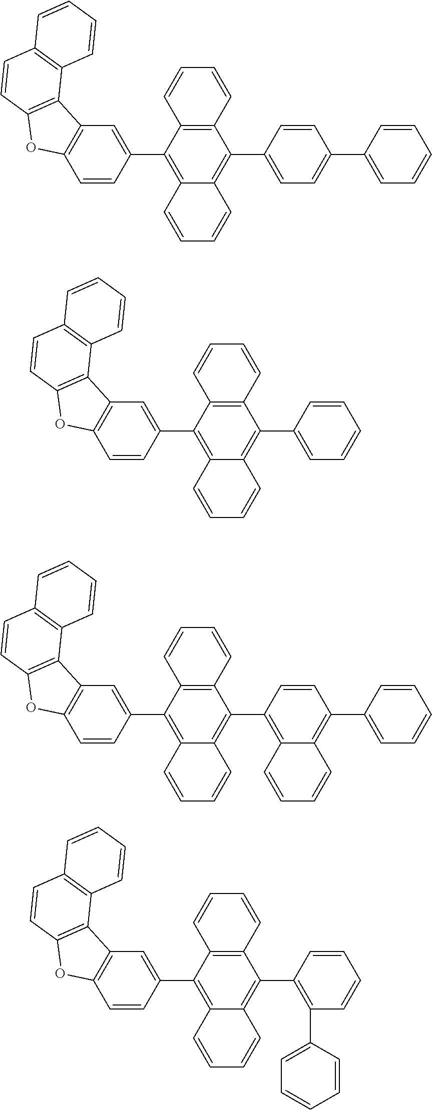 [SMILES: c1ccc(-c2ccc(-c3c4ccccc4c(-c4ccc5oc6ccc7ccccc7c6c5c4)c4ccccc34)cc2)cc1 ; c1ccc(-c2c3ccccc3c(-c3ccc4oc5ccc6ccccc6c5c4c3)c3ccccc23)cc1 ; c1ccc(-c2ccc(-c3c4ccccc4c(-c4ccc5oc6ccc7ccccc7c6c5c4)c4ccccc34)c3ccccc23)cc1 ; c1ccc(-c2ccccc2-c2c3ccccc3c(-c3ccc4oc5ccc6ccccc6c5c4c3)c3ccccc23)cc1]

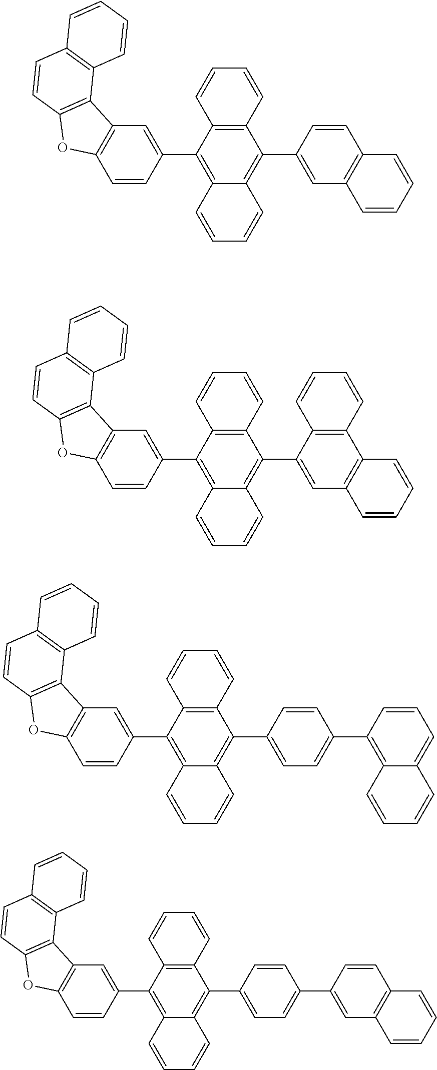 [SMILES: c1ccc2cc(-c3c4ccccc4c(-c4ccc5oc6ccc7ccccc7c6c5c4)c4ccccc34)ccc2c1 ; c1ccc2c(c1)cc(-c1c3ccccc3c(-c3ccc4oc5ccc6ccccc6c5c4c3)c3ccccc13)c1ccccc12 ; c1ccc2c(-c3ccc(-c4c5ccccc5c(-c5ccc6oc7ccc8ccccc8c7c6c5)c5ccccc45)cc3)cccc2c1 ; c1ccc2cc(-c3ccc(-c4c5ccccc5c(-c5ccc6oc7ccc8ccccc8c7c6c5)c5ccccc45)cc3)ccc2c1]

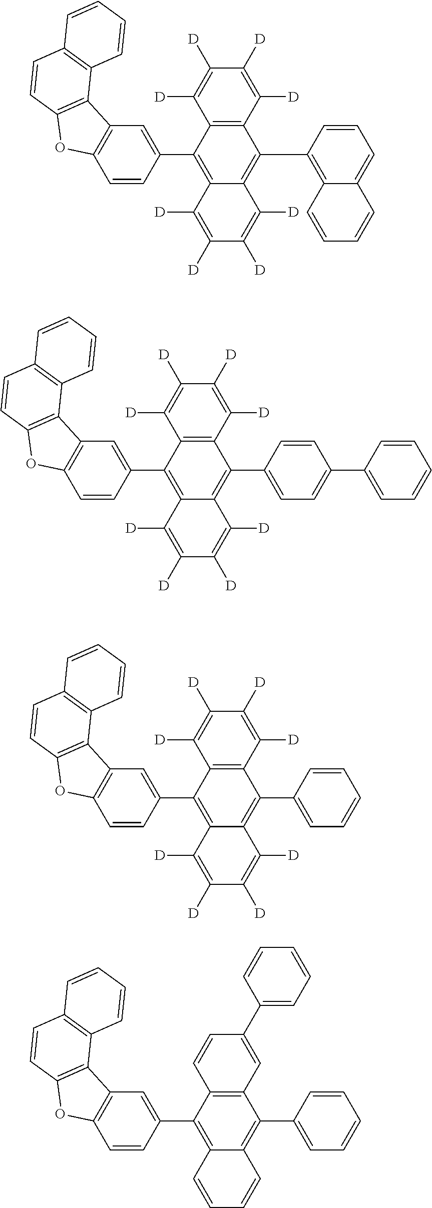

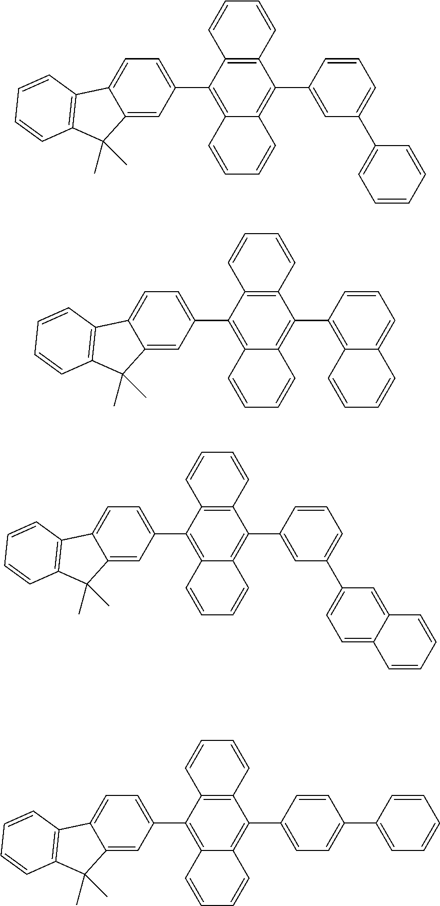

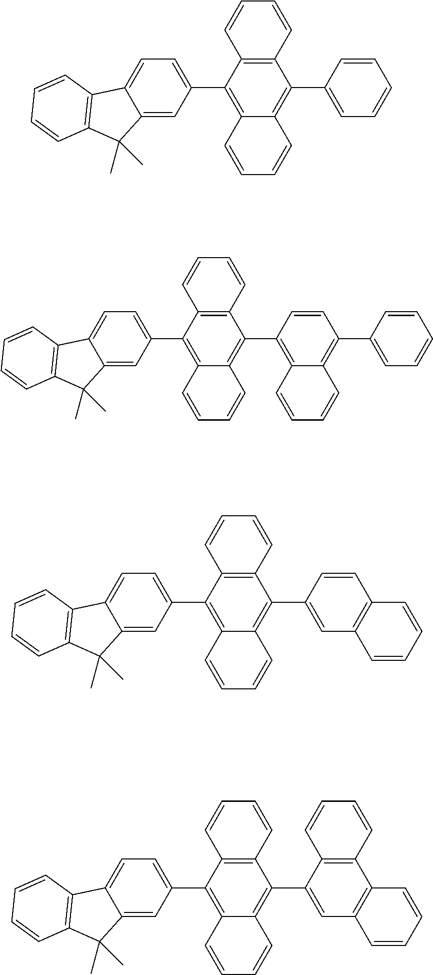

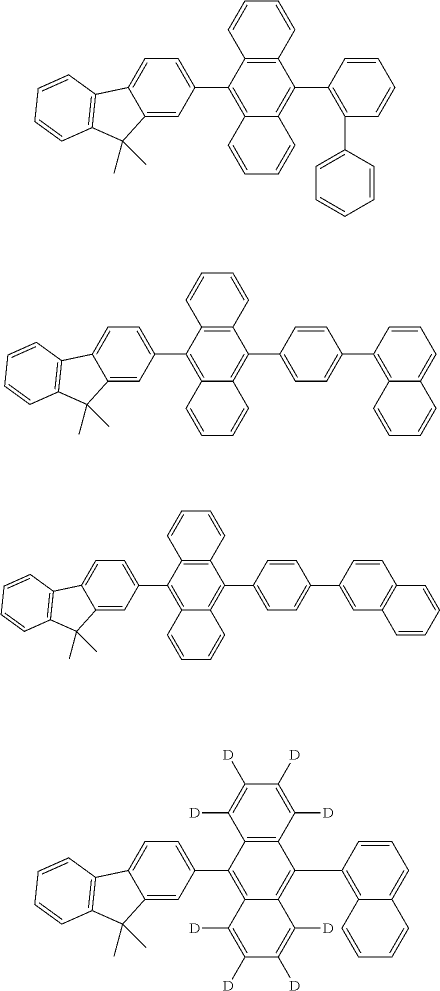

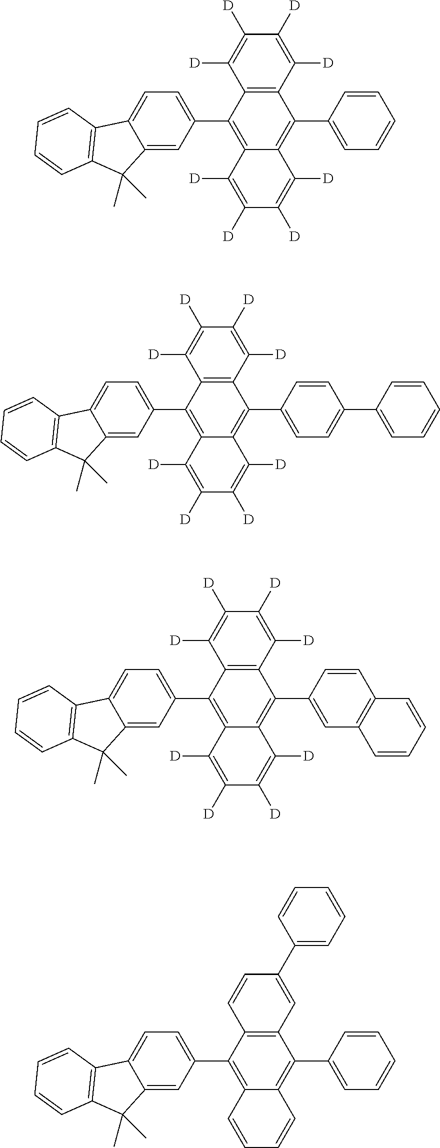

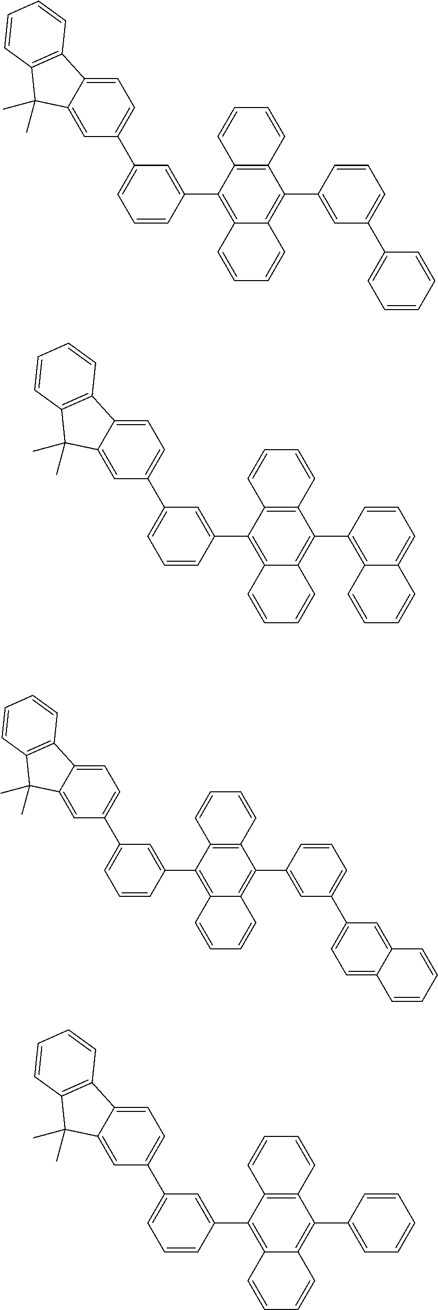

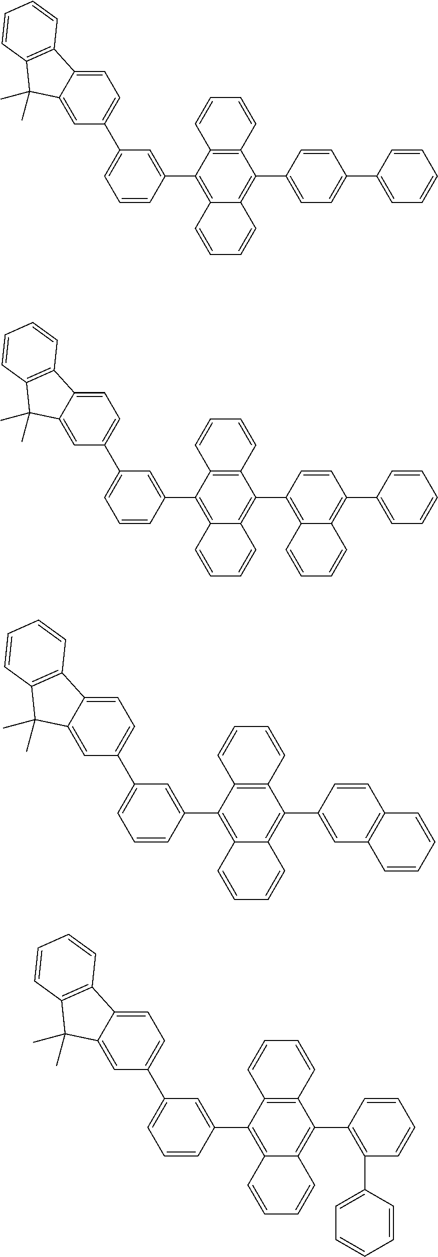

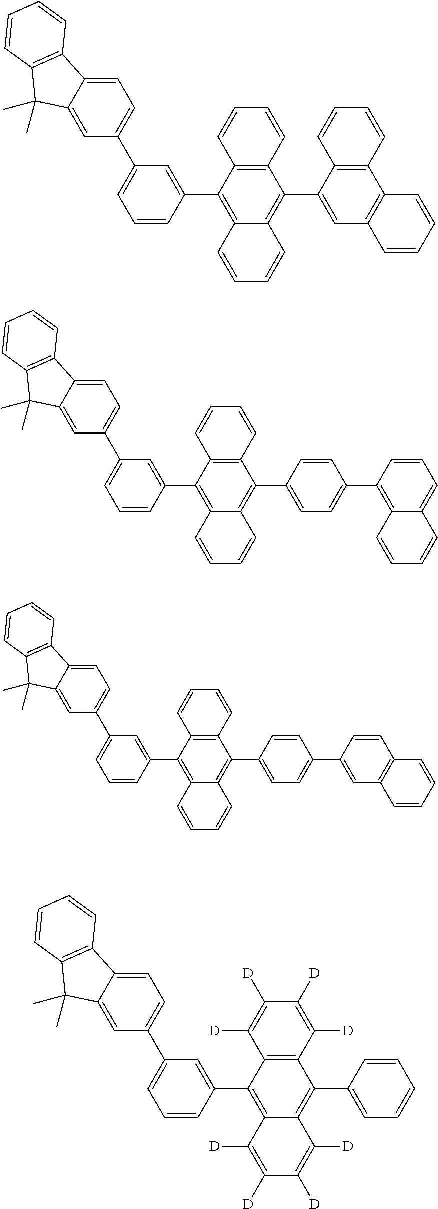

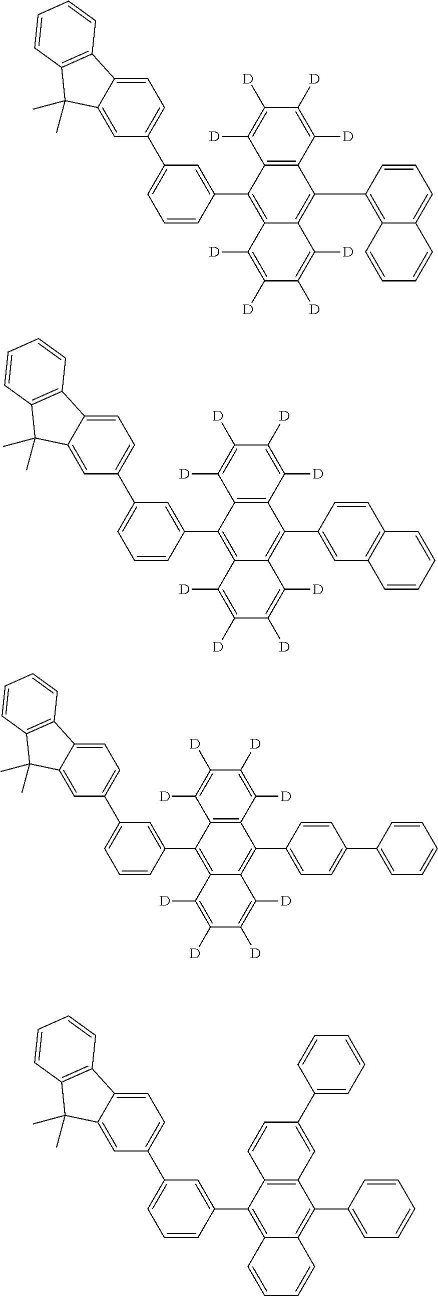

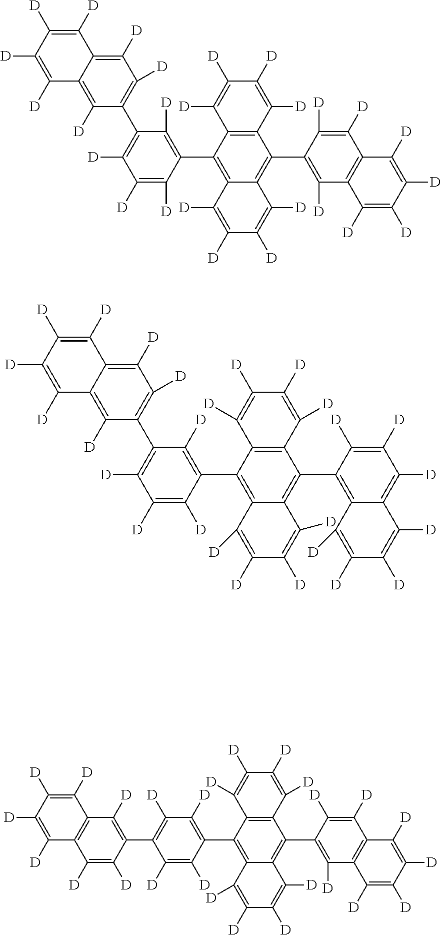

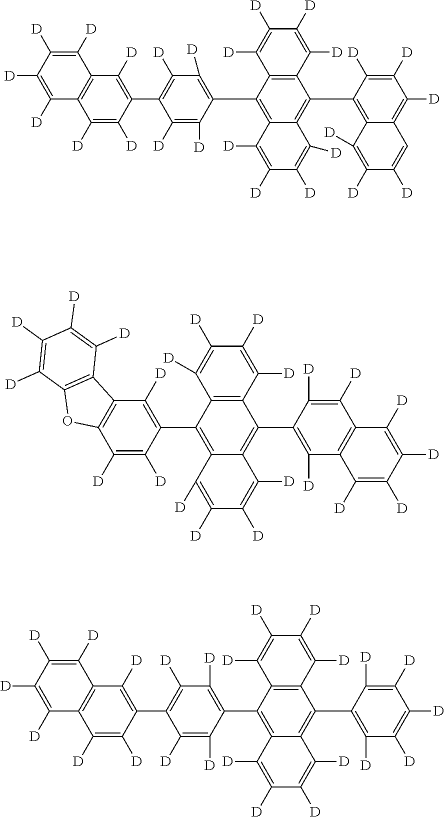

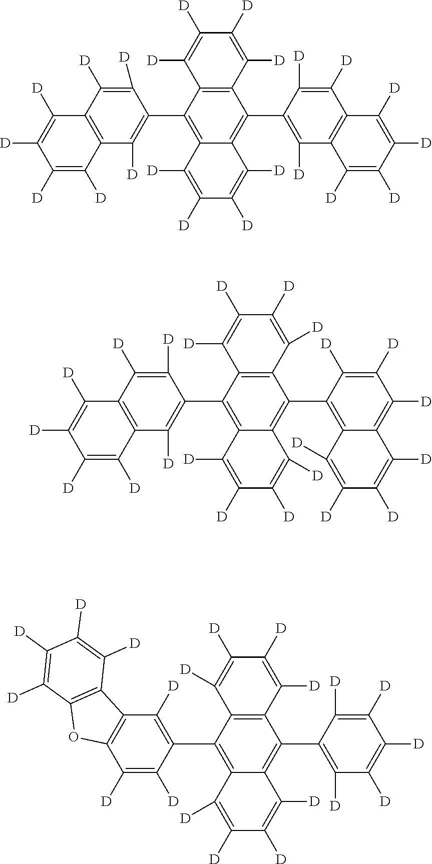

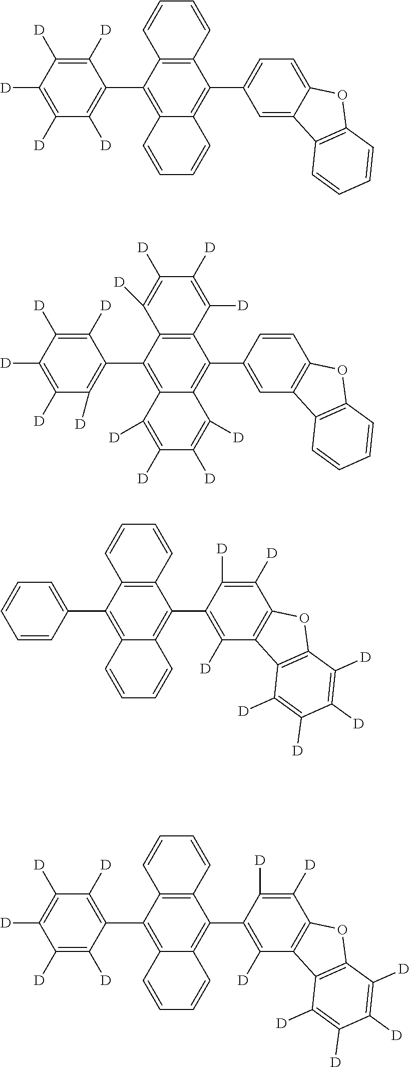 [SMILES: [2H]c1c([2H])c([2H])c(-c2c3ccccc3c(-c3ccc4oc5ccccc5c4c3)c3ccccc23)c([2H])c1[2H] ; [2H]c1c([2H])c([2H])c(-c2c3c([2H])c([2H])c([2H])c([2H])c3c(-c3ccc4oc5ccccc5c4c3)c3c([2H])c([2H])c([2H])c([2H])c23)c([2H])c1[2H] ; [2H]c1c([2H])c([2H])c2c(oc3c([2H])c([2H])c(-c4c5ccccc5c(-c5ccccc5)c5ccccc45)c([2H])c32)c1[2H] ; [2H]c1c([2H])c([2H])c(-c2c3ccccc3c(-c3c([2H])c([2H])c4oc5c([2H])c([2H])c([2H])c([2H])c5c4c3[2H])c3ccccc23)c([2H])c1[2H]]

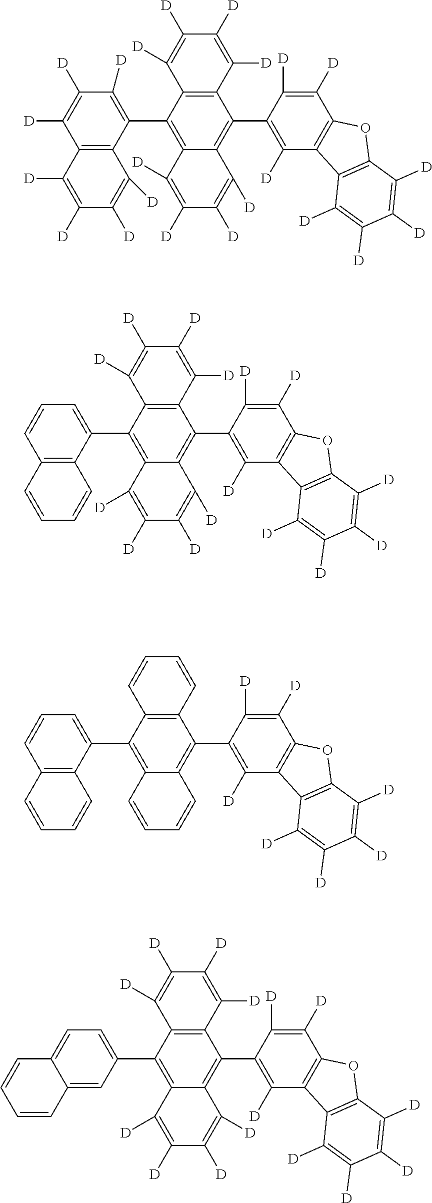

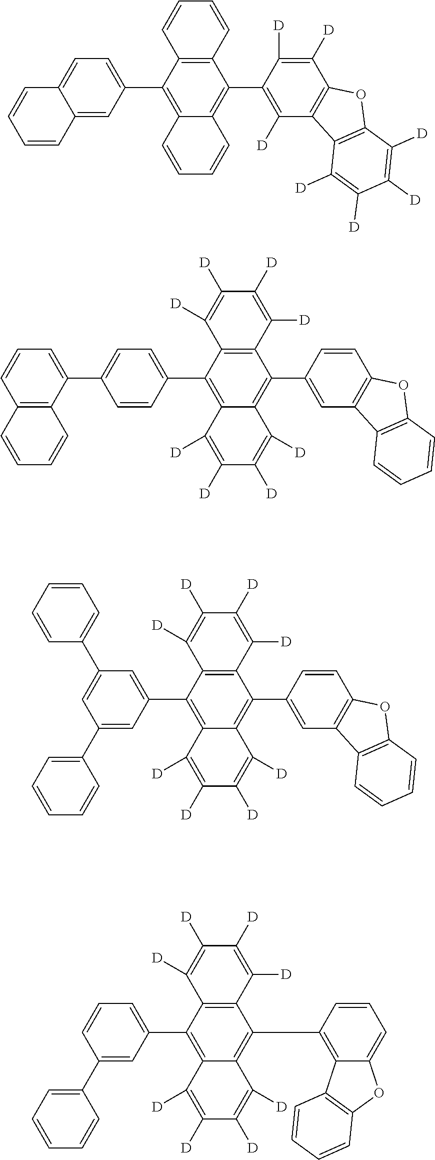 [SMILES: [2H]c1c([2H])c([2H])c2c(oc3c([2H])c([2H])c(-c4c5ccccc5c(-c5ccc6ccccc6c5)c5ccccc45)c([2H])c32)c1[2H] ; [2H]c1c([2H])c([2H])c2c(-c3ccc4oc5ccccc5c4c3)c3c([2H])c([2H])c([2H])c([2H])c3c(-c3ccc(-c4cccc5ccccc45)cc3)c2c1[2H] ; [2H]c1c([2H])c([2H])c2c(-c3ccc4oc5ccccc5c4c3)c3c([2H])c([2H])c([2H])c([2H])c3c(-c3cc(-c4ccccc4)cc(-c4ccccc4)c3)c2c1[2H] ; [2H]c1c([2H])c([2H])c2c(-c3cccc4oc5ccccc5c34)c3c([2H])c([2H])c([2H])c([2H])c3c(-c3cccc(-c4ccccc4)c3)c2c1[2H]]

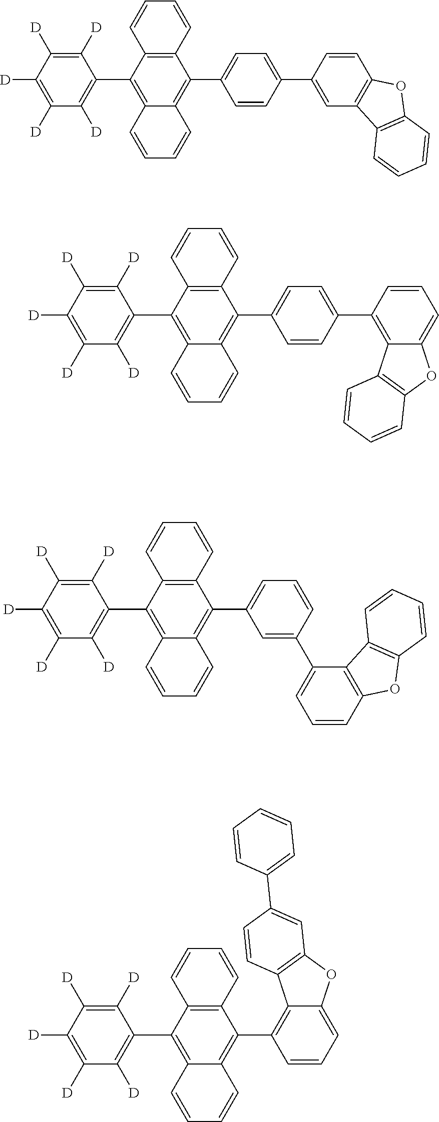 [SMILES: [2H]c1c([2H])c([2H])c(-c2c3ccccc3c(-c3ccc(-c4ccc5oc6ccccc6c5c4)cc3)c3ccccc23)c([2H])c1[2H] ; [2H]c1c([2H])c([2H])c(-c2c3ccccc3c(-c3ccc(-c4cccc5oc6ccccc6c45)cc3)c3ccccc23)c([2H])c1[2H] ; [2H]c1c([2H])c([2H])c(-c2c3ccccc3c(-c3cccc(-c4cccc5oc6ccccc6c45)c3)c3ccccc23)c([2H])c1[2H] ; [2H]c1c([2H])c([2H])c(-c2c3ccccc3c(-c3cccc4oc5cc(-c6ccccc6)ccc5c34)c3ccccc23)c([2H])c1[2H]]

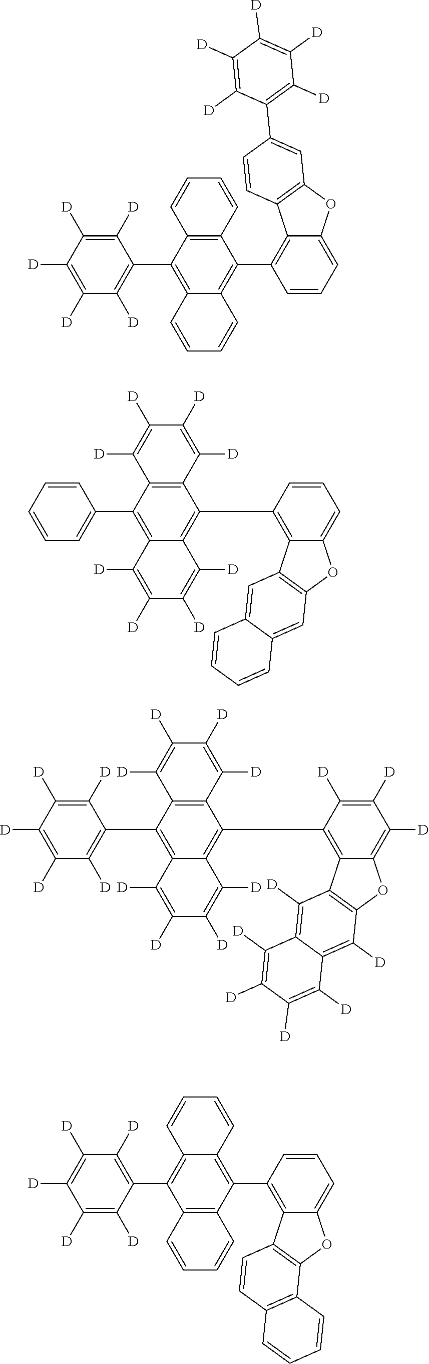

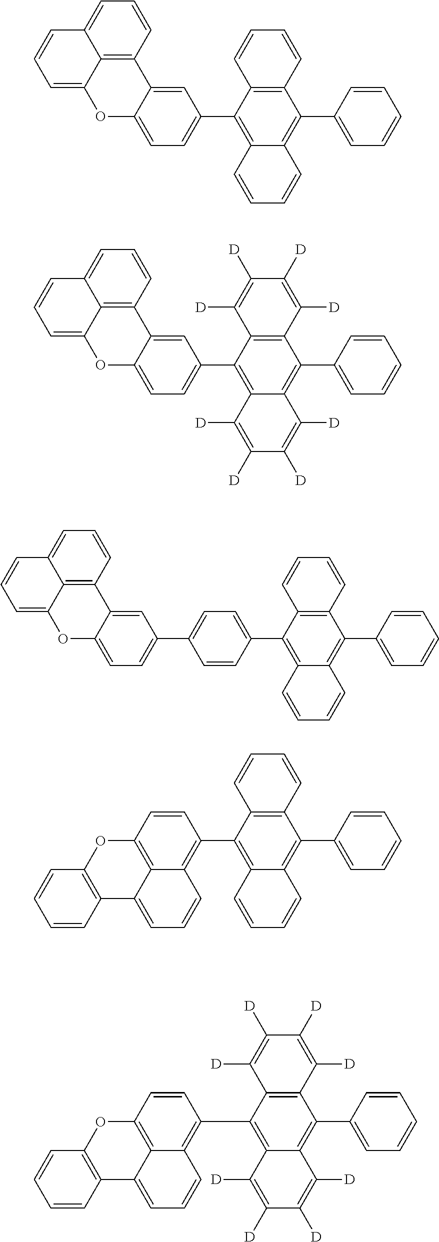

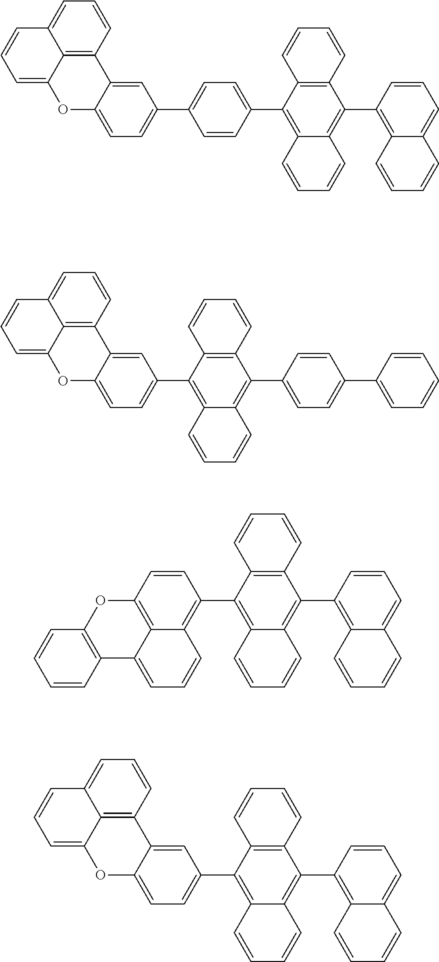 [SMILES: c1ccc2c(-c3c4ccccc4c(-c4ccc(-c5ccc6c(c5)-c5cccc7cccc(c57)O6)cc4)c4ccccc34)cccc2c1 ; c1ccc(-c2ccc(-c3c4ccccc4c(-c4ccc5c(c4)-c4cccc6cccc(c46)O5)c4ccccc34)cc2)cc1 ; c1ccc2c(c1)Oc1ccc(-c3c4ccccc4c(-c4cccc5ccccc45)c4ccccc34)c3cccc-2c13 ; c1ccc2c(-c3c4ccccc4c(-c4ccc5c(c4)-c4cccc6cccc(c46)O5)c4ccccc34)cccc2c1]

As described above, the organic EL device according to an aspect of the present invention includes a cathode, an anode, and an organic layer arranged between the cathode and the anode, wherein the organic layer includes the compound represented by the formula (1); except that, conventionally-known materials and device configurations can be applied, as long as the effect of the present invention is not impaired.

An organic EL device according to a first embodiment includes a cathode, an anode, and an emitting layer arranged between the cathode and the anode, wherein the emitting layer includes the compound represented by the formula (1).

The amount of the compound represented by the formula (1) in the emitting layer is preferably 1% by mass or more and 20% by mass or less based on the entire emitting layer.

In one embodiment, the emitting layer includes a second compound which is not the same as a first compound represented by the formula (1).

In one embodiment, the second compound is a heterocyclic compound or a fused aromatic compound.

In one embodiment, the second compound is an anthracene derivative.

In one embodiment, the second compound is the compound represented by the formula (10).

An organic EL device according to a second embodiment includes a cathode, an anode, and a first emitting layer and a second emitting layer arranged between the cathode and the anode in this order from the anode side, wherein the first emitting layer includes the compound represented by the formula (1).

The amount of the compound represented by the formula (1) in the emitting layer is preferably 1% by mass or more and 20% by mass or less based on the entire first emitting layer.

The first emitting layer includes a first host material and a first dopant material. The second emitting layer includes a second host material and a second dopant material. The first host material and the second host material are different from each other. The first dopant material and the second dopant material are the same as or different from each other.

The organic EL device according to the second embodiment includes at least two emitting layers (first emitting layer and second host material). The first emitting layer according to the second embodiment has the same constitution as that of the emitting layer according to the first embodiment.

The content different from that of the first embodiment will be mainly described below, and the overlap of the content is omitted or simplified.

When the Tripret-Tripret-Annhilation (sometimes referred to as TTA) is used in the organic EL device according to the second embodiment, the long lifetime can be achieved and the luminous efficiency can be improved.

The TTA is a mechanism that a triplet exciton and another triplet exciton are collided to form a singlet exciton. The TTA mechanism is sometimes referred to as the TTF mechanism as described in WO 2010/134350 A1.

The TTF phenomenon will be described. A hole injected from the anode and an electron injected from the cathode are recombined in the emitting layer to form an exciton. The spin state has the ratio of 25% singlet excitons and 75% triplet excitons as conventionally known. In the conventionally-known fluorescent device, the 25% singlet excitons are relieved to the ground state to emit light, but the remaining 75% triplet excitons are returned to the ground state without emitting light through a thermal inactivation process. Accordingly, it has been referred that theoretical limit value of internal quantum efficiency of the conventionally fluorescent device is 25%.

On the other hand, behavior of the triplet exciton formed inside an organic matter has been theoretically investigated. According to S. M. Bachilo et al (J. Phys. Chem. A, 104, 7711 (2000)), in the case that it is assumed that high-order exciton such as a quintet exciton is returned to the triplet exciton soon, when the density of the triplet exciton (hereinafter, described as ³A*) is increased, triplet excitons each other are collided and a reaction like the following formula happens. Here, ¹A represents a ground state, and ¹A* represents a lowest excited singlet exciton.

³A*+³A*→(4/9)¹A+(1/9)¹A*+(13/9)³A*

That is, it is the reaction 5³A*→4¹A+¹A*, and it is predicted that 1/5, that is, 20% of 75% triplet excitons originally formed are changed to singlet excitons. Accordingly, the amount of the singlet exciton contributed as light is 40% in which the value 75%×(1/5)=15% are added to the value 25% originally formed. At this time, the emission proportion derived from the TTF (TTF rate) in the entire emission strength is 15/40, that is, 37.5%. Further, when 75% triplet excitons originally formed are collided each other to form a singlet exciton (one singlet exciton is formed from two triplet excitons), extremely high internal quantum efficiency being 62.5% in which the value 75%×(1/2)=37.5% are added to the 25% singlet excitons originally formed can be obtained. In this case, the TTF rate is the value 37.5/62.5=60%.

In the organic EL device according to the second embodiment, in terms of expressing the TTF mechanism, the triplet energy T₁ (H1) of the first host material and the triplet energy T₁ (H2) of the second host material preferably satisfy the relationship of the following numerical formula (Numerical formula 1), and more preferably the relationship of the following numerical formula (Numerical formula 2).

T₁(H2)>T₁(H1) (Numerical formula 1)

T₁(H2)−T₁(H1)>0.03 eV (Numerical formula 2)

When the relationship of the numerical formula (Numerical formula 1) is satisfied in the organic EL device according to the second embodiment, regarding the triplet excitons generated by recombination of holes and electrons in the second emitting layer, even when excess carriers exist at the interface between the second emitting layer and the organic layer that is in direct contact therewith, it is considered that the triplet excitons present at the interface between the second emitting layer and the organic layer are less likely to be quenched. For example, quenching by excess electrons is possible when recombination regions are locally present at the interface between the second emitting layer and the hole-transporting layer or electron-barrier layer. On the other hand, when a recombination region exists locally at the interface between the first emitting layer and the electron-transporting layer or the hole-barrier layer, quenching by excess holes is possible.

The organic EL device according to the second embodiment includes the first emitting layer and the second emitting layer so as to satisfy the relationship of the numerical formula (Numerical formula 1), so that the triplet excitons generated in the second emitting layer move to the first emitting layer without being quenched by excess carriers, and can be prevented from migrating back from the first emitting layer to the second emitting layer. As a result, the TTF mechanism is developed in the first emitting layer, singlet excitons are efficiently generated, and the luminous efficiency is improved.

In this way, the organic EL device includes the second emitting layer that mainly generates triplet excitons, and the first emitting layer that mainly expresses the TTF mechanism by utilizing the triplet excitons that have moved from the second emitting layer as different regions, using a compound having a lower triplet energy than the second host material in the second emitting layer as the first host material in the first emitting layer, the luminous efficiency is improved by providing a difference in triplet energy.

When the organic EL device according to the second embodiment selects a combination of host materials that satisfy the relationship of the numerical formula (Numerical formula 1), and the first emitting layer includes the first compound (compound represented by the formula (1)) according to the first embodiment, the lifetime of the device can be extended and further, the luminous efficiency can be improved.

### (Emission Wavelength of Organic EL Device)

The organic EL device according to the second embodiment preferably emits light having the maximum peak wavelength of 500 nm or less in driving the device, and more preferably emits light of 420 nm or more and 480 nm or less. The measurement of the maximum peak wavelength of the light emitted by the organic EL device in driving the device can be conducted using the method described in the Examples.

### (First Emitting Layer)

The first emitting layer includes the first host material and the first dopant material. The first host material is different from the second host material included in the second emitting layer.

In the organic EL device according to the second embodiment, the first emitting layer preferably emits light having the maximum peak wavelength of 500 nm or less in driving the device.

The measurement of the maximum peak wavelength of the light emitted by the emitting layer in driving the device can be conducted using the method described as follows.

Maximum peak wavelength λp of light emitted from emitting layer in driving device

Regarding the Maximum peak wavelength λp₁ of the light emitted from the first emitting layer in the driving device, the organic EL device is fabricated using the same material as that of the first emitting layer for forming the second emitting layer, and when a voltage is applied to the device so that the current density of the organic EL device became 10 mA/cm², the spectral emission luminance spectrum is measured by using Spectroradiometer CS-2000 (manufactured by KONICA MINOLTA, INC.). The maximum peak wavelength λp₁ (unit: nm) is calculated from the obtained spectral emission luminance spectrum.

Regarding the Maximum peak wavelength λp₂ of the light emitted from the second emitting layer in the driving device, the organic EL device is fabricated using the same material as that of the second emitting layer for forming the first emitting layer, and when a voltage is applied to the device so that the current density of the organic EL device became 10 mA/cm², the spectral emission luminance spectrum is measured by using Spectroradiometer CS-2000 (manufactured by KONICA MINOLTA, INC.). The maximum peak wavelength λp₂ (unit: nm) is calculated from the obtained spectral emission luminance spectrum.

In the organic EL device according to the second embodiment, the half width FWHM of the maximum peak of the first dopant material is preferably 1 nm or more and 20 nm or less.

In the organic EL device according to the second embodiment, the Stokes shift of the first dopant material preferably exceeds 7 nm.

When the Stokes shift of the first dopant material exceeds 7 nm, a decrease in the luminous efficiency due to self-absorption is likely to be prevented.

The self-absorption is a phenomenon in which the same compound absorbs the emitted light, and a phenomenon in which a decrease in luminous efficiency is caused. Since the self-absorption is conspicuously observed in a compound with a small Stokes shift (that is, the overlap between an absorption spectrum and a fluorescence spectrum is large), a compound with a large Stokes shift (the overlap between an absorption spectrum and a fluorescence spectrum is small) is preferably used to suppress the self-absorption. The Stokes shift can be measured using the method described as follows.

A compound being a measurement target is dissolved in toluene at a concentration of 2.0×10⁵ mol/L to prepare a sample for measurement. A measurement sample placed in a quartz cell is irradiated with continuous light in the ultraviolet-visible region at room temperature (300 K) to measure an absorption spectrum (vertical axis: absorbance, horizontal axis: wavelength). A spectrophotometer can be used for absorption spectrum measurement, and for example, the Spectrophotometer U-3900/3900H manufactured by Hitachi High-Tech Science Corporation can be used. Further, a compound being a measurement target is dissolved in toluene at a concentration of 4.9×10⁻⁶ mol/L to prepare a sample for measurement. A measurement sample placed in a quartz cell was irradiated with excitation light at room temperature (300 K) to measure a fluorescence spectrum (vertical axis: fluorescence intensity, horizontal axis: wavelength). A spectrophotometer can be used for fluorescence spectrum measurement, and for example, the Fluorescence Spectrophotometer F-7000 manufactured by Hitachi High-Tech Science Corporation can be used.

The difference between the maximum absorption wavelength and the maximum fluorescence wavelength is calculated using these absorption spectra and fluorescence spectra to determine the Stokes shift (SS). The unit of Stokes shift SS is nm.

In the organic EL device according to the second embodiment, the triplet energy T₁ (D1) of the first dopant material and the triplet energy T₁ (H1) of the first host material preferably satisfy the relationship of the following numerical formula (Numerical formula 4A).

T₁(D1)>T₁(H1) (Numerical formula 4A)

When the first dopant material and the first host material satisfy the relationship of the following numerical formula (Numerical formula 4A) in the organic EL device according to the second embodiment, in the case where the triplet excitons generated in the second emitting layer move to the first emitting layer, energy thereof is transferred to the molecule of the first host material rather than to the first dopant material having a higher triplet energy. Further, a triplet exciton generated by recombination of a hole and an electron on the first host material do not move to the first dopant material with higher triplet energy. The triplet exciton generated by recombination on the molecule of the first dopant material quickly transfer energy to the molecule of the first host material.

When triplet excitons efficiently collide with each other on the first host material by the TTF phenomenon without the triplet excitons of the first host material migrating to the first dopant material, a singlet exciton is produced.

In the organic EL device according to the second embodiment, the singlet energy S₁ (H1) of the first host material and the singlet energy S₁ (D1) of the first dopant material preferably satisfy the relationship of the following numerical formula (Numerical formula 4).

S₁(H1)>S₁(D1) (Numerical formula 4)

When the first dopant material and the first host material satisfy the relationship of the numerical formula (Numerical formula 4) in the organic EL device according to the second embodiment, since the singlet energy of the first dopant material is less than the singlet energy of the first host material, the singlet exciton generated by the TTF phenomenon transfer energy from the first host material to the first dopant material to contribute to the emission (preferably fluorescent emission) of the first dopant material.

### (Singlet Energy S₁)

As a method for measuring singlet energy S₁ using a solution (sometimes referred to as a solution method), the following methods can be given.

10⁵ mol/L or more and 10⁻⁴ mol/L or less of a toluene solution of compound being a measurement target is prepared as a sample, it is put in a quartz cell, and then an absorption spectrum of the sample is measured at room temperature (300 K) (vertical axis: absorption strength and horizontal axis: wavelength). A tangent line is drawn to the fall on the long wavelength side of this absorption spectrum, and then the wavelength value λedge [nm] at the intersection of the tangent line and the horizontal axis is substituted into the following conversion formula (F2) to calculate the singlet energy.

S₁[eV]=1239.85/λedge Conversion formula (F2)

As an absorption spectrum measuring apparatus, for example, a spectrophotometer (apparatus name: U3310) manufactured by Hitachi High-Tech Science Corporation can be used, but it is not limited to this.

A tangent to the fall on the long wavelength side of the absorption spectrum is drawn as follows. Among the maximum values of the absorption spectrum, the tangent line at each point on the curve is considered when moving from the maximum value on the longest wavelength side to the long wavelength direction on the spectrum curve. Regarding this tangent line, the slope is decreased, and then it is increased as the curve falls (that is, as the vertical value decreases). The tangent line drawn at the point where the value of the slope takes the minimum value on the long wavelength side (provided that the case where the absorbance is 0.1 or less is excluded) is taken as the tangent line to the falling edge on the long wavelength side of the absorption spectrum.

A maximum point with an absorbance value of 0.2 or less is not included in the maximum value on the longest wavelength side.

In the organic EL device according to the second embodiment, when the stacking order of the first emitting layer and the second emitting layer is the order of the second emitting layer and the first emitting layer from the anode side, the electron mobility μe (H2) of the second host material and the electron mobility μe (H1) of the first host material preferably satisfy the relationship of the following numerical formula (Numerical formula 3). When the first host material and the second host material satisfy the relationship of the following numerical formula (Numerical formula 3), the ability to recombine a hole and an electron in the second emitting layer is improved.

μe(H1)>μe(H2) (Numerical formula 3)

In the organic EL device according to the second embodiment, when the stacking order of the first emitting layer and the second emitting layer is the order of the second emitting layer and the first emitting layer from the anode side, the hole mobility μh (H2) of the second host material and the hole mobility μh (H1) of the first host material also preferably satisfy the relationship of the following numerical formula (Numerical formula 31).

μh(H2)>μh(H1) (Numerical formula 31)

In the organic EL device according to the second embodiment, when the stacking order of the first emitting layer and the second emitting layer is the order of the second emitting layer and the first emitting layer from the anode side, the hole mobility μh (H2) of the second host material, the electron mobility μe (H2) of the second host material, the hole mobility μh (H1) of the first host material and the electron mobility μe (H1) of the first host material also preferably satisfy the relationship of the following numerical formula (Numerical formula 32).

(μe(H1)/μh(H1))>(μe(H2)/μh(H2)) (Numerical formula 32)

The electron mobility can be measured by the following method using impedance spectroscopy.

A measurement target layer with a thickness of 100 nm to 200 nm is sandwiched between the anode and the cathode, and a minute alternate current voltage of 100 mV or less is applied while applying a bias DC voltage. The value of alternate current (absolute value and phase) flowing at this time is measured. This measurement is performed while changing the frequency of the AC voltage, and a complex impedance (Z) is calculated using the current value and the voltage value. At this time, the frequency dependence of the imaginary part (ImM) of the modulus M=iwZ (i: imaginary unit, w: angular frequency) is obtained, and the reciprocal of the frequency ω at which the ImM reaches the maximum value is defined as the response time of an electron conducting in the measurement target layer. Then, the electron mobility is calculated using the following formula.

Electron mobility=(Thickness of measurement target layer)²/(Response time−Voltage)

The hole mobility can be measured in the same manner as the electron mobility using impedance spectroscopy.

The hole mobility is calculated using the following formula.

Hole mobility=(Thickness of measurement target layer)²/(Response time−Voltage)

In the organic EL device according to the second embodiment, the amount of the first dopant material in the first emitting layer is preferably the same range as the amount of the first dopant material described in the first embodiment.

In the organic EL device according to the second embodiment, the amount of the first host material in the first emitting layer is preferably the same range as the amount of the first host material described in the first embodiment.

In the organic EL device according to the second embodiment, the first emitting layer preferably has the thickness of 5 nm or more, and more preferably 15 nm or more. When the first emitting layer has the thickness of 5 nm or more, it is likely to be suppressed that a triplet exciton moved from the second emitting layer to the first emitting layer is returned to the second emitting layer again. Further, when the first emitting layer has the thickness of 5 nm or more, the triplet exciton can sufficiently be separated from the recombination moiety in the second emitting layer.

In the organic EL device according to the second embodiment, the first emitting layer preferably has the thickness of 20 nm or less. When the first emitting layer has the thickness of 20 nm or less, the TTF phenomenon can be likely to further happen by improving the density of the triplet exciton in the first emitting layer.

In the organic EL device according to the second embodiment, the first emitting layer preferably has the thickness of 5 nm or more and 20 nm or less.

(Second emitting layer) The second emitting layer includes the second host material and the second dopant material.

The second host material is different from the first host material included in the first emitting layer.

The second dopant material is preferably a compound in which the maximum peak wavelength represents an emission of 500 nm or less. The second dopant material is more preferably a compound in which the maximum peak wavelength represents a fluorescent emission of 500 nm or less.

A method for measuring the maximum peak wavelength of the compound is the same as described above.

In the organic EL device according to the second embodiment, the second dopant material and the first dopant material are the same or different compounds.

In the organic EL device according to the second embodiment, the second emitting layer does not preferably include a metal complex. Further, in the organic EL device according to the present embodiment, it is also preferable that the first emitting layer do not include a boron-containing complex.

In the organic EL device according to the second embodiment, the second emitting layer does not preferably include a fluorescent emitting material (dopant material).

Further, the second emitting layer does not preferably include a heavy metal complex and a phosphorescent emitting rare earth metal complex.

In the emission spectrum of the second dopant material, when a peak of the emission strength with the maximum is defined as the maximum peak and the height of the maximum peak is defined as 1, the height of another peak found in the emission spectrum is preferably less than 0.6.

The peak of the emission spectrum is used as the local maximal value.

Further, in the emission spectrum of the second emitting compound, the number of the peaks is preferably less than 3.

In the organic EL device according to the present embodiment, the second emitting layer preferably emits light having the maximum peak wavelength of 500 nm or less in driving the device.

(Second host material) As the second host material, for example, 1) a fused aromatic compound such as an anthracene derivative, a phenanthrene derivative, a pyrene derivative, a benzanthracene derivative, a fluorene derivative, a fluoranthene derivative, and a chrysene derivative, 2) a heterocyclic compound such as a carbazole derivative, a dibenzofuran derivative, a dibenzothiophene derivative, and a benzoxanthene derivative can be given.

The second host material is preferably a fused aromatic compound, and more preferably a pyrene derivative (a compound represented by the general formula (100) described later).

Further, the second host material is also preferably a benzanthracene derivative (a compound represented by the general formula (1X) described later) or a benzoxanthene derivative (a compound represented by the general formula (14X) described later).

When the second host material is a pyrene derivative, the second host material is preferably a compound represented by the following general formula (100).

(Compound represented by the general formula (100))

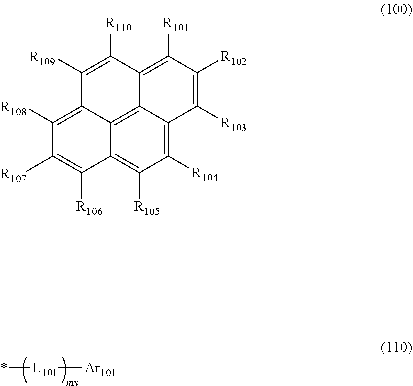

In the general formula (100),
R₁₀₁ to R₁₁₀ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms,
a substituted or unsubstituted heterocyclic group having 5 to 50 ring atoms, or
the group represented by the general formula (110);
provided that at least one of R₁₀₁ to R₁₁₀ is the group represented by the general formula (110);
when a plurality of groups represented by the general formula (110) is present, the plurality of groups represented by the general formula (110) is the same as or different from each other;
L₁₀₁ is
a single bond,
a substituted or unsubstituted arylene group having 6 to 50 ring carbon atoms, or
a substituted or unsubstituted divalent heterocyclic group having 5 to 50 ring atoms;
Ar₁₀₁ is
a substituted or unsubstituted aryl group having 6 to 50 ring carbon atoms, or
a substituted or unsubstituted heterocyclic group having 5 to 50 ring atoms;
mx is 0, 1, 2, 3, 4 or 5;
when two or more L₁₀₁'s are present, the two or more L₁₀₁'s are the same as or different from each other;
when two or more Ar₁₀₁'s are present, the two or more Ar₁₀₁'s are the same as or different from each other; and
* in the general formula (110) represents a bonding position with the pyrene ring in the general formula (100).

In the general formula (100), R₉₀₁, R₉₀₂, R₉₀₃, R₉₀₄, R₉₀₅, R₉₀₆, R₉₀₇, R₈₀₁ and R₈₀₂ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms;
when a plurality of R₉₀ₗ's is present, the plurality of R₉₀ₗ's is the same as or different from each other;
when a plurality of R₉₀₂'s is present, the plurality of R₉₀₂'s is the same as or different from each other;
when a plurality of R₉₀₃'s is present, the plurality of R₉₀₅'s is the same as or different from each other;
when a plurality of R₉₀₄'s is present, the plurality of R₉₀₄'s is the same as or different from each other;
when a plurality of R₉₀₅'s is present, the plurality of R₉₀₅'s is the same as or different from each other;
when a plurality of R₉₀₆'s is present, the plurality of R₉₀₆'s is the same as or different from each other;
when a plurality of R₉₀₇'s is present, the plurality of R₉₀₇'s is the same as or different from each other;
when a plurality of R₈₀₁'s is present, the plurality of R₈₀₁'s is the same as or different from each other; and
when a plurality of R₈₀₂'s is present, the plurality of R₈₀₂'s is the same as or different from each other.

In the organic EL device according to the present embodiment, the group represented by the general formula (110) is preferably a group represented by the following general formula (111).

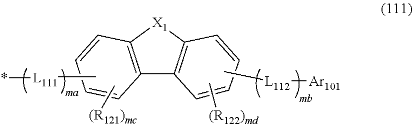

In the general formula (111),
X1 is CR₁₂₃R₁₂₄, an oxygen atom, a sulfur atom, or NR₁₂₅;
L₁₁₁ and L₁₁₂ are independently
a single bond,
a substituted or unsubstituted arylene group having 6 to 50 ring carbon atoms, or
a substituted or unsubstituted divalent heterocyclic group having 5 to 50 ring atoms;
ma is 0, 1, 2, 3 or 4;
mb is 0, 1, 2, 3 or 4;
ma+mb is 0, 1, 2, 3 or 4;
Ar₁₀₁ is the same as defined for Ar₁₀₁ in the general formula (110);
R₁₂₁, R₁₂₂, R₁₂₃, R₁₂₄ and R₁₂₅ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
mc is 3;
the three R₁₂₁'s are the same as or different from each other;
md is 3; and
the three R₁₂₂'s are the same as or different from each other.

L₁₁₁ is bonded with any one position of carbon atoms *1 to *4 in positions of carbon atoms *1 to *8 in a ring structure represented by the following general formula (111a) of the group represented by the general formula (111), R₁₂₁ is bonded with the remaining three positions of carbon atoms *1 to *4, L₁₁₂ is bonded with any one position of carbon atoms *5 to *8 and R₁₂₂ is bonded with the remaining three positions of carbon atoms *5 to *8.

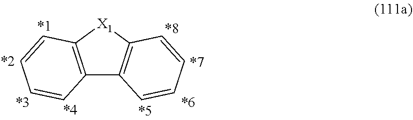

For example, in the group represented by the general formula (111), when L₁₁₁ is bonded with the position of the carbon atom *2 in the ring structure represented by the general formula (111a) and L₁₁₂ is bonded with the position of the carbon atom *7 in the ring structure represented by the general formula (111a), the group represented by the general formula (111) is represented by the following general formula (111b).

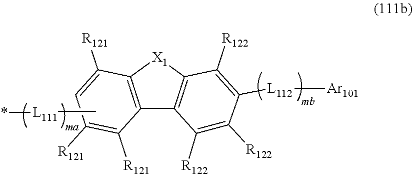

In the general formula (111b),
X₁, L₁₁₁, L₁₁₂, ma, mb, Ar₁₀₁, R₁₂₁, R₁₂₂, R₁₂₃, R₁₂₄ and R₁₂₅ are independently the same as defined for X1, L₁₁₁, L₁₁₂, ma, mb, Ar₁₀₁, R₁₂₁, R₁₂₂, R₁₂₃, R₁₂₄ and R₁₂₅ in the general formula (111);
a plurality of R₁₂₁'s is the same as or different from each other; and
a plurality of R₁₂₂'s is the same as or different from each other.

In the organic EL device according to the present embodiment, the group represented by the general formula (111) is preferably the group represented by the general formula (111b).

In the organic EL device according to the present embodiment,
it is preferable that ma be 0, 1 or 2; and
mb be 0, 1 or 2.

In the organic EL device according to the present embodiment,
it is preferable that ma be 0 or 1; and
mb be 0 or 1.

In the organic EL device according to the present embodiment, Ar₁₀₁ is preferably a substituted or unsubstituted aryl group having 6 to 50 ring carbon atoms.

In the organic EL device according to the present embodiment,
Ar₁₀₁ is preferably
a substituted or unsubstituted phenyl group,
a substituted or unsubstituted naphthyl group,
a substituted or unsubstituted biphenyl group,
a substituted or unsubstituted terphenyl group,
a substituted or unsubstituted pyrenyl group,
a substituted or unsubstituted phenanthryl group, or
a substituted or unsubstituted fluorenyl group.

In the organic EL device according to the present embodiment,
Ar₁₀₁ is also preferably a group represented by the following general formula (120), general formula (130) or general formula (140).

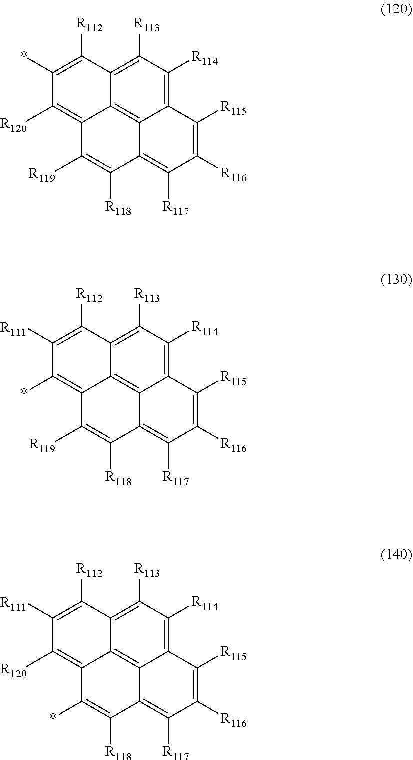

In the general formula (120), the general formula (130) and the general formula (140),
R₁₁₁ to R₁₂₀ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a group represented by —N(R₉₀₆)(R₉₀₇),
a substituted or unsubstituted aralkyl group having 7 to 50 carbon atoms,
a group represented by —C(═O)R₁₂₄,
a group represented by —COOR₁₂₅,
a halogen atom,
a cyano group,
a nitro group,
a substituted or unsubstituted aryl group having 6 to 50 ring carbon atoms, or
a substituted or unsubstituted heterocyclic group having 5 to 50 ring atoms; and
* in the general formula (120), the general formula (130) and the general formula (140) represents a bonding position with L₁₀₁ in the general formula (110), or a bonding position with L₁₁₂ in the general formula (111) or the general formula (111b).

In the organic EL device according to the present embodiment,
the second host material is preferably represented by the following general formula (101).

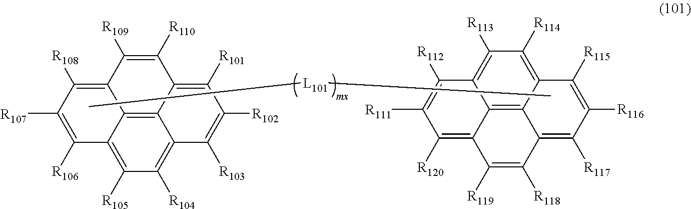

In the general formula (101),
R₁₀₁ to R₁₂₀ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
provided that one of R₁₀₁ to R₁₁₀ is represents a bonding position with L₁₀₁, and one of R₁₁₁ to R₁₂₀ is represents a bonding position with L₁₀₁;
L₁₀₁ is
a single bond,
a substituted or unsubstituted arylene group having 6 to 50 ring carbon atoms, or
a substituted or unsubstituted divalent heterocyclic group having 5 to 50 ring atoms;
mx is 0, 1, 2, 3, 4 or 5; and
when two or more L₁₀₁'s are present, the two or more L₁₀₁'s are the same as or different from each other.

In the organic EL device according to the present embodiment,
L₁₀₁ is preferably
a single bond, or
a substituted or unsubstituted arylene group having 6 to 50 ring carbon atoms.

In the organic EL device according to the present embodiment, the second host material is preferably represented by the following general formula (102).

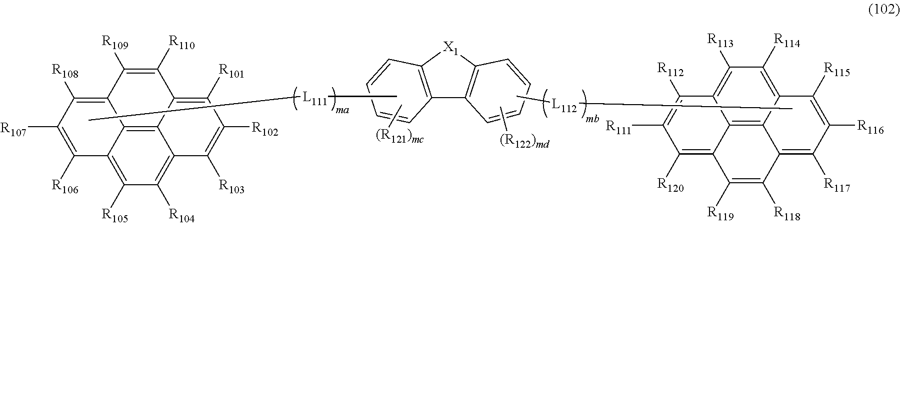

In the general formula (102),
R₁₀₁ to R₁₂₀ are independently the same as defined for R₁₀₁ to R₁₂₀ in the general formula (101);
provided that one of R₁₀₁ to R₁₁₀ is represents a bonding position with L₁₁₁, and one of R₁₁₁ to R₁₂₀ is represents a bonding position with L₁₁₂;
X1 is CR₁₂₃R₁₂₄, an oxygen atom, a sulfur atom, or NR₁₂₅;
L₁₁₁ and L₁₁₂ are independently
a single bond,
a substituted or unsubstituted arylene group having 6 to 50 ring carbon atoms, or
a substituted or unsubstituted divalent heterocyclic group having 5 to 50 ring atoms;
ma is 0, 1, 2, 3 or 4;
mb is 0, 1, 2, 3 or 4;
ma+mb is 0, 1, 2, 3 or 4;
R₁₂₁, R₁₂₂, R₁₂₃, R₁₂₄ and R₁₂₅ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
mc is 3;
the three R₁₂₁'s are the same as or different from each other;
md is 3; and
the three R₁₂₂'s are the same as or different from each other.

In the compound represented by the general formula (102),
it is preferable that ma be 0, 1 or 2, and
mb be 0, 1 or 2.

In the compound represented by the general formula (102),
it is preferable that ma be 0 or 1, and
mb be 0 or 1.

In the organic EL device according to the present embodiment,
two or more of R₁₀₁ to R₁₁₀ are preferably the group represented by the general formula (110).

In the organic EL device according to the present embodiment,
it is preferable that two or more of R₁₀₁ to R₁₁₀ be the group represented by the general formula (110), and Ar₁₀₁ is a substituted or unsubstituted aryl group having 6 to 50 ring carbon atoms.

In the organic EL device according to the present embodiment,
it is preferable that Ar₁₀₁ be not a substituted or unsubstituted pyrenyl group,
L₁₀₁ be not a substituted or unsubstituted pyrenylene group, and
a substituted or unsubstituted aryl group having 6 to 50 ring carbon atoms for R₁₀₁ to R₁₁₀ which are not the group represented by the general formula (110) be not a substituted or unsubstituted pyrenyl group.

In the organic EL device according to the present embodiment,
it is preferable that R₁₀₁ to R₁₁₀ which are not the group represented by the general formula (110) be independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms.

In the organic EL device according to the present embodiment,
it is preferable that R₁₀₁ to R₁₁₀ which are not the group represented by the general formula (110) be independently
a hydrogen atom,
a substituted or unsubstituted alkyl group having 1 to 50 carbon atoms, or
a substituted or unsubstituted cycloalkyl group having 3 to 50 ring carbon atoms.

In the organic EL device according to the present embodiment, R₁₀₁ to R₁₁₀ which are not the group represented by the general formula (110) are preferably hydrogen atoms.

In the compound represented by the general formula (100), all groups described as “substituted or unsubstituted” are preferably an “unsubstituted” group.

The compound represented by the general formula (100) can be produced by a known method.

(Specific examples of compound represented by the general formula (100))

Specific examples of the compound represented by the general formula (100) include the following compounds. Here, the compound represented by the general formula (100) is not limited to the following specific examples.

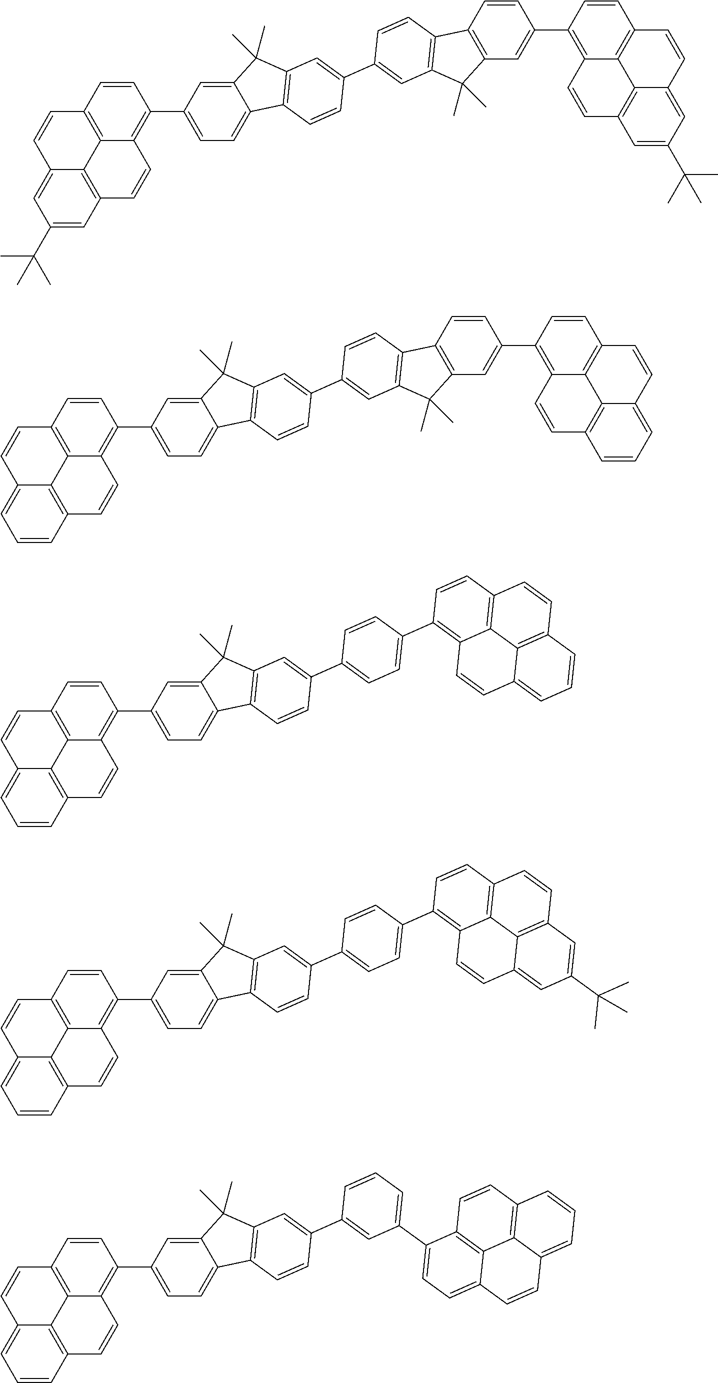

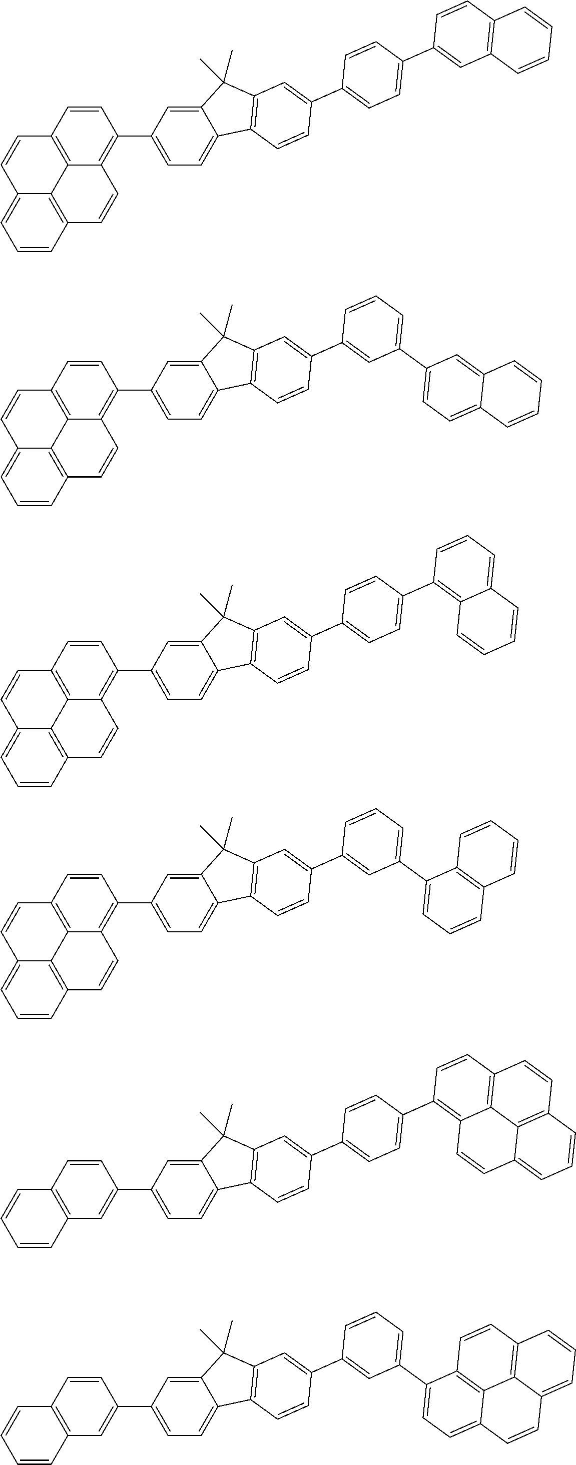

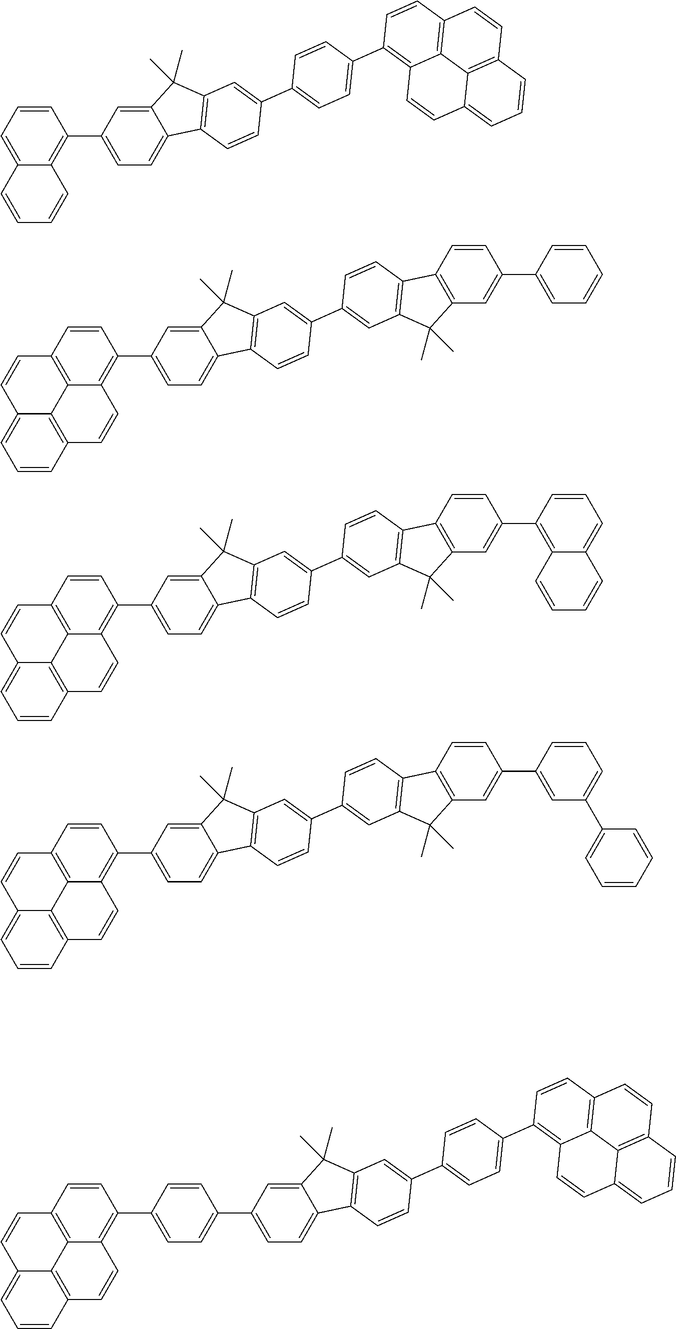

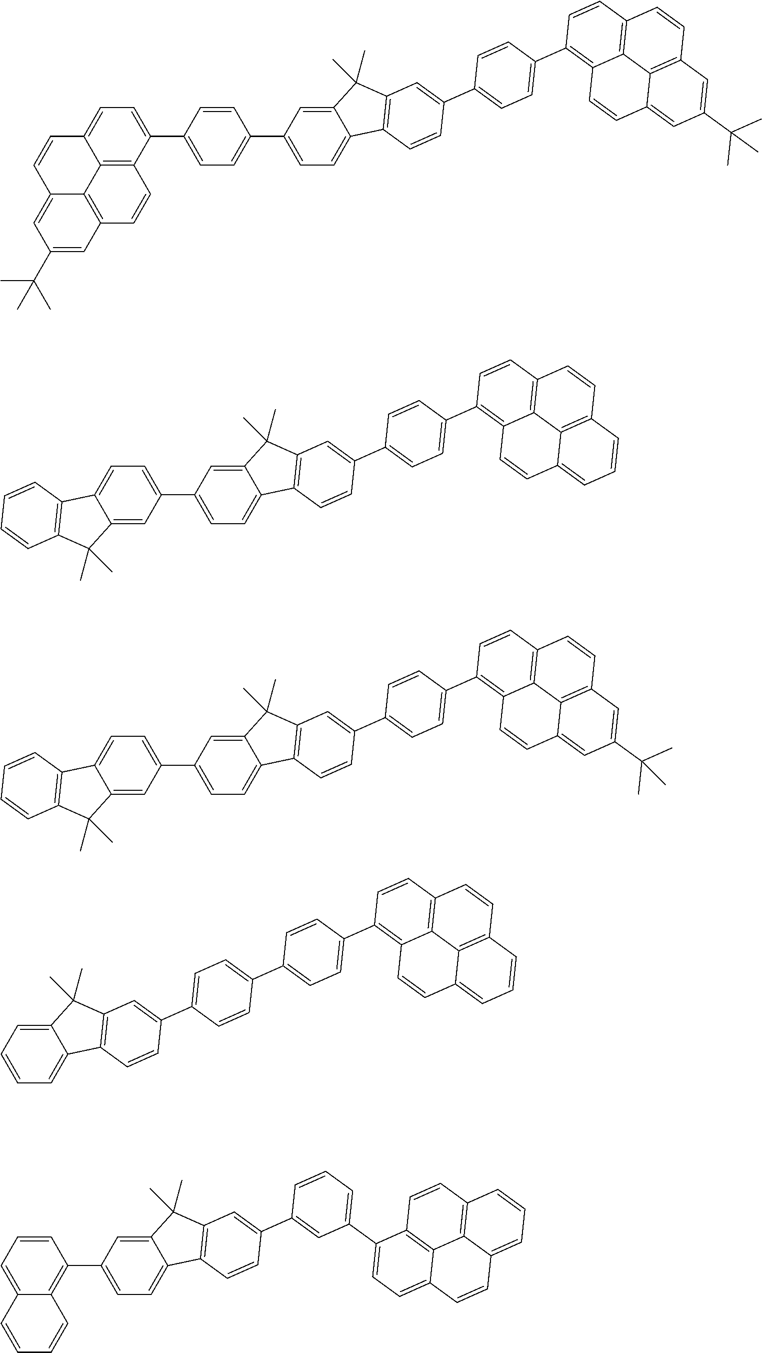

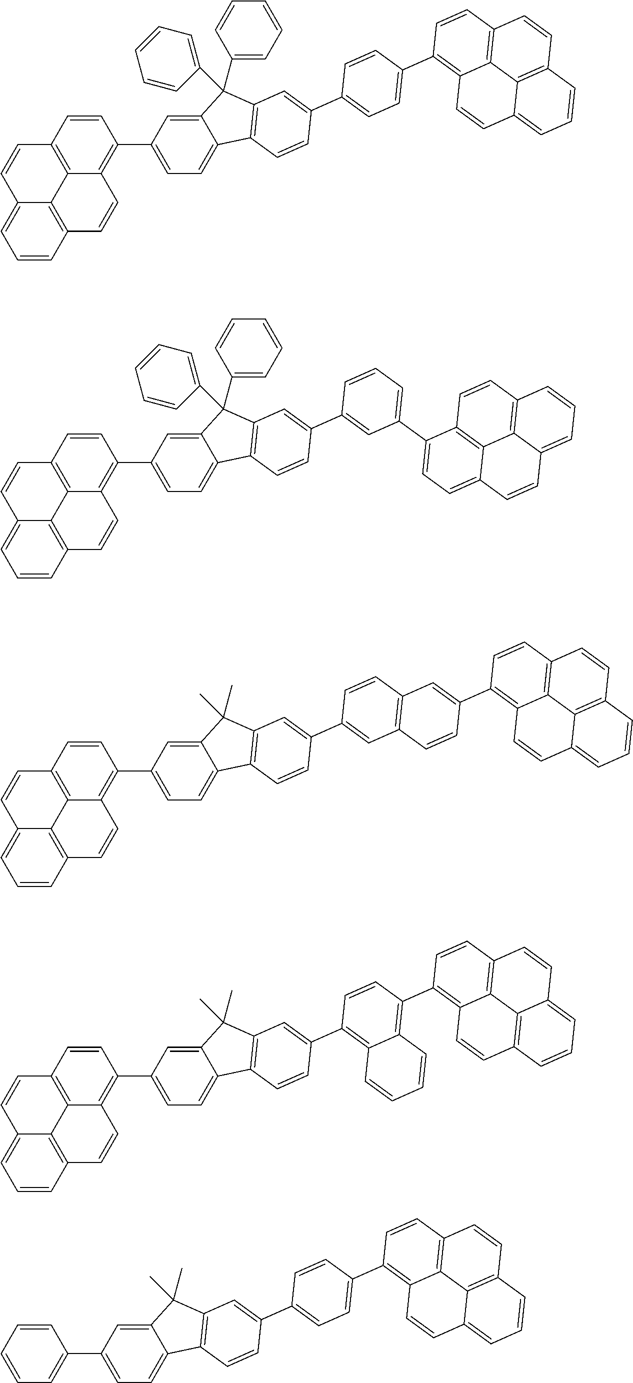

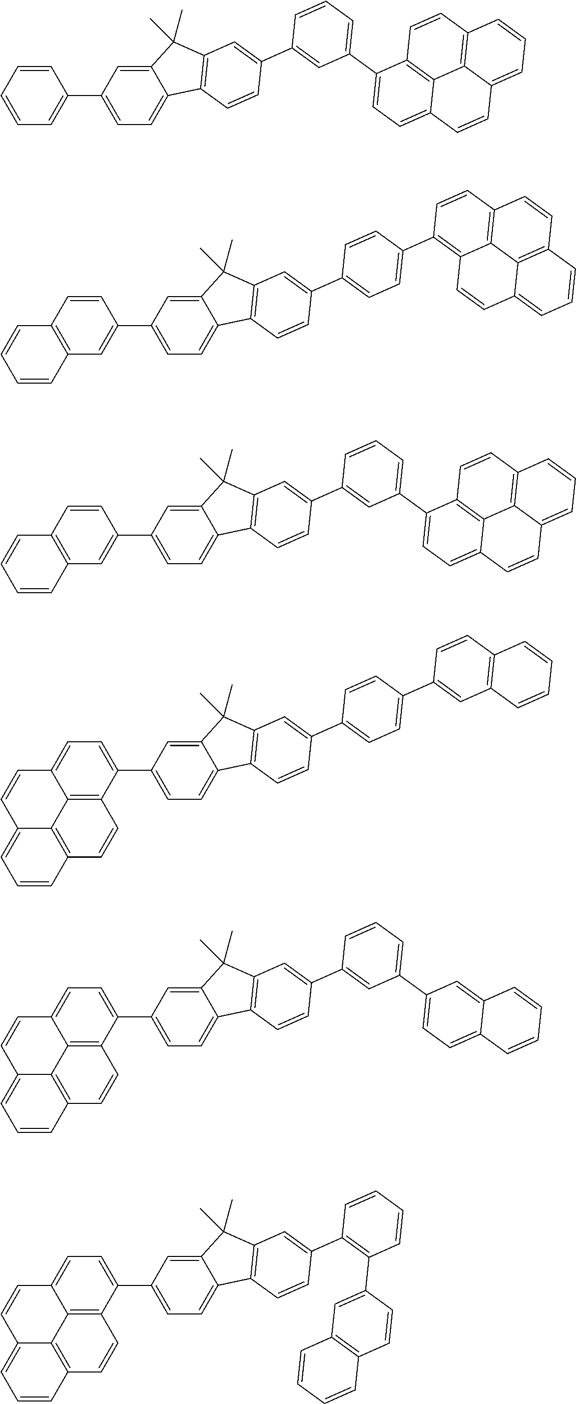

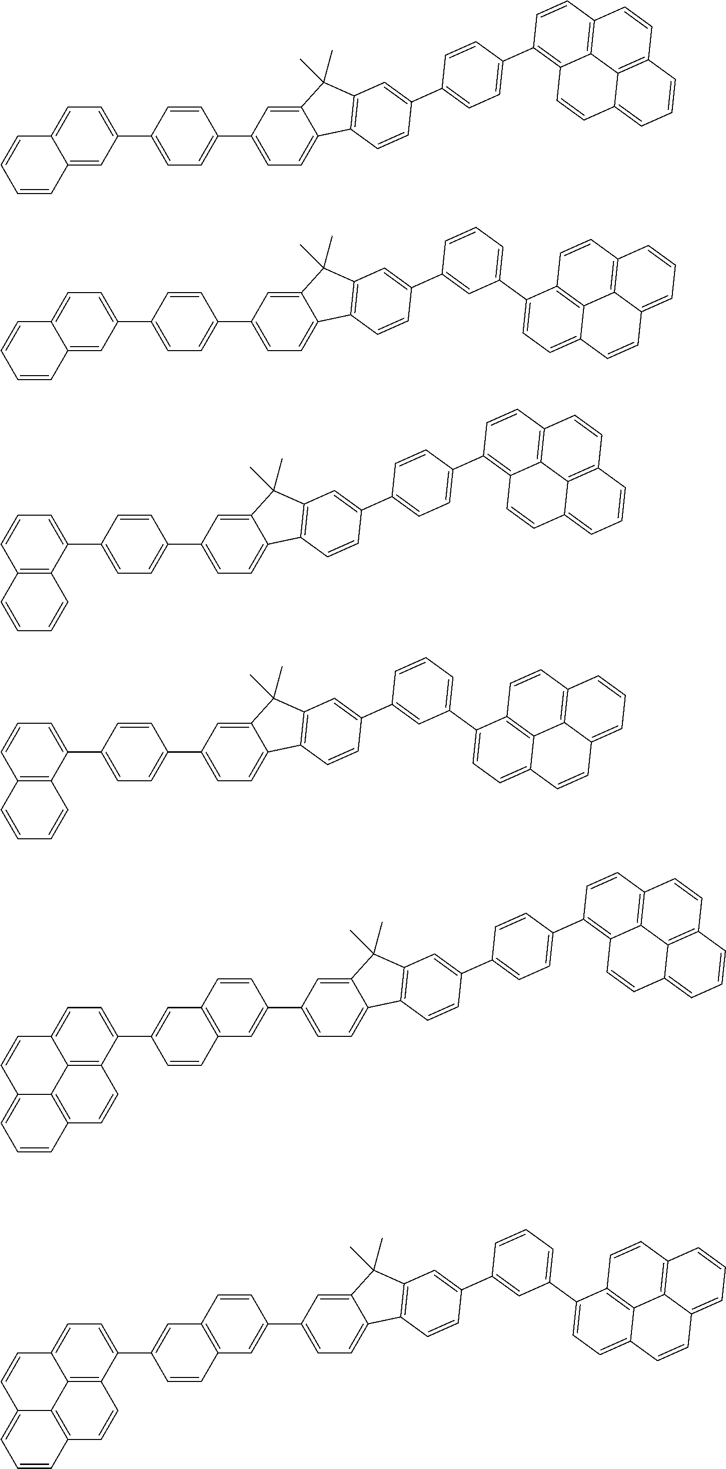

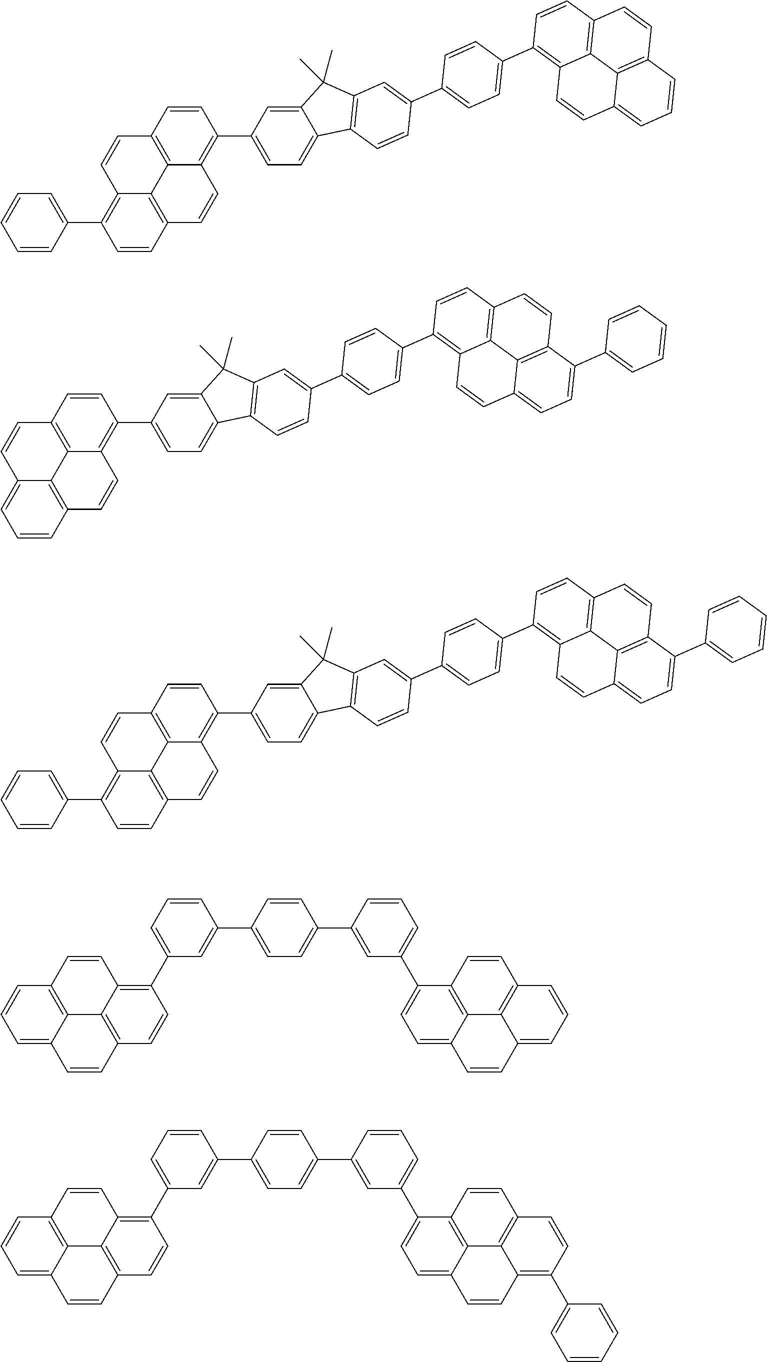

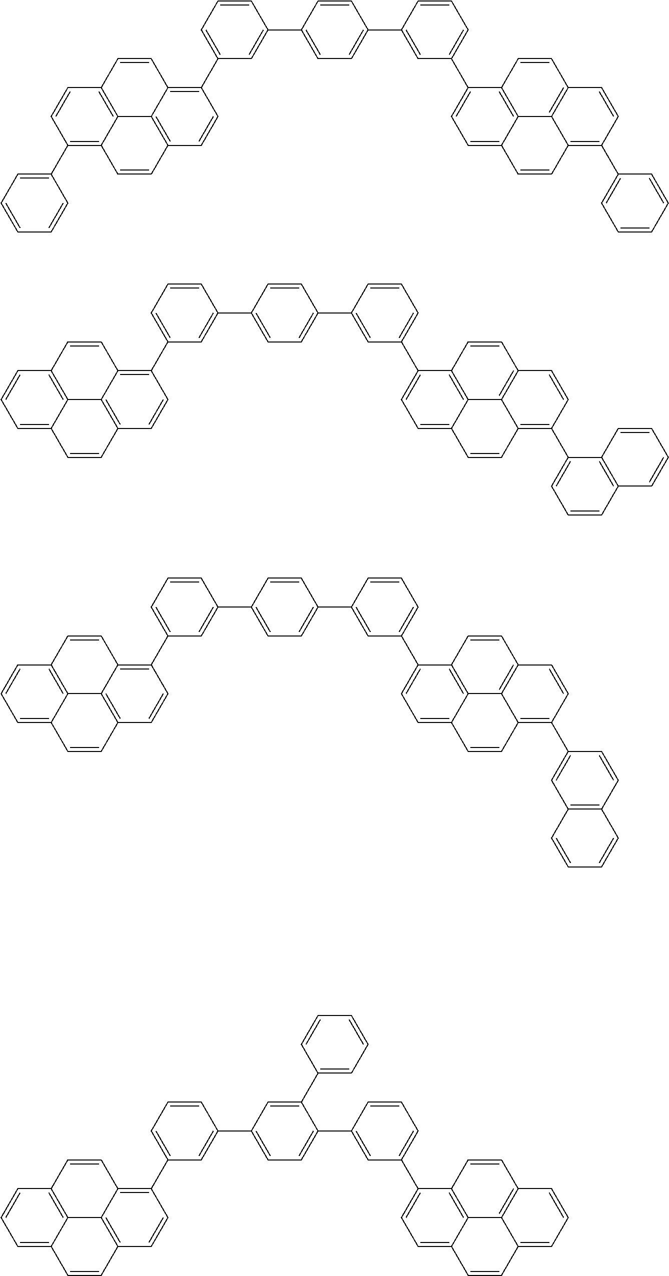

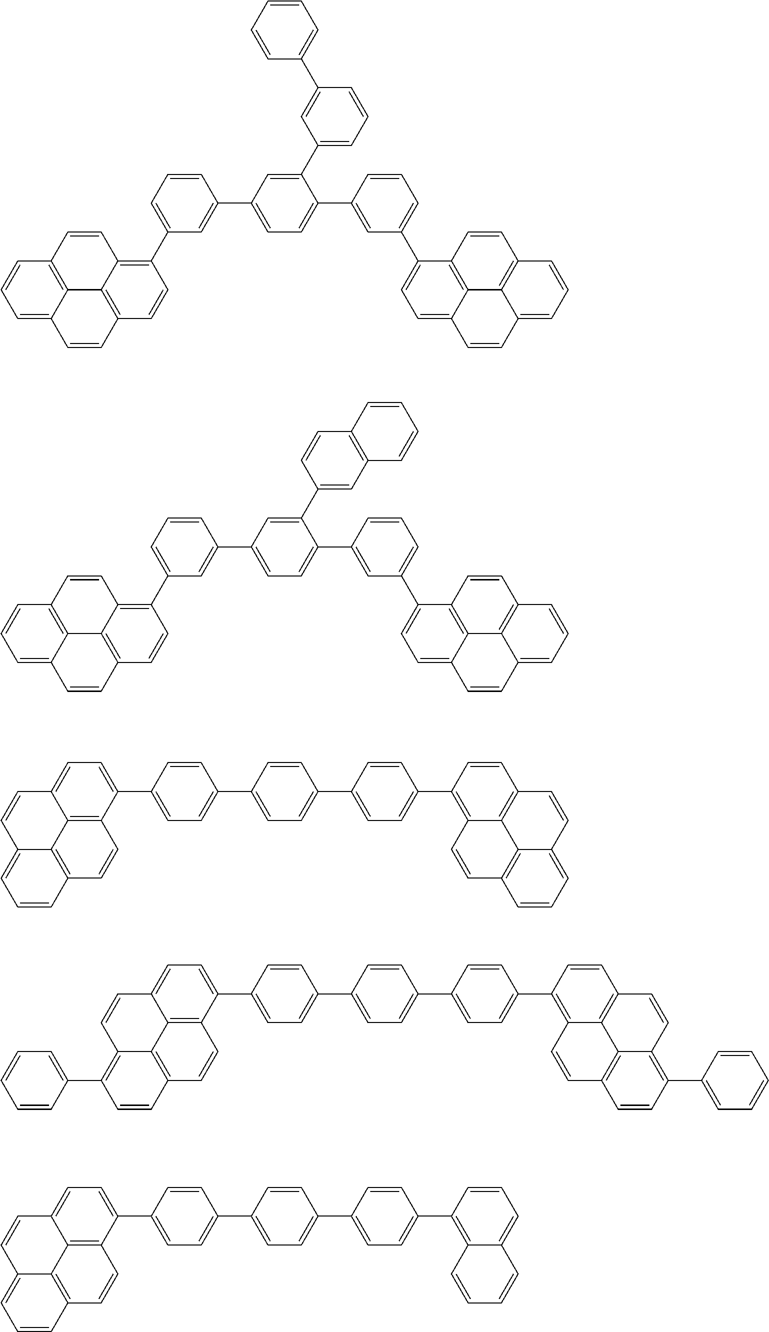

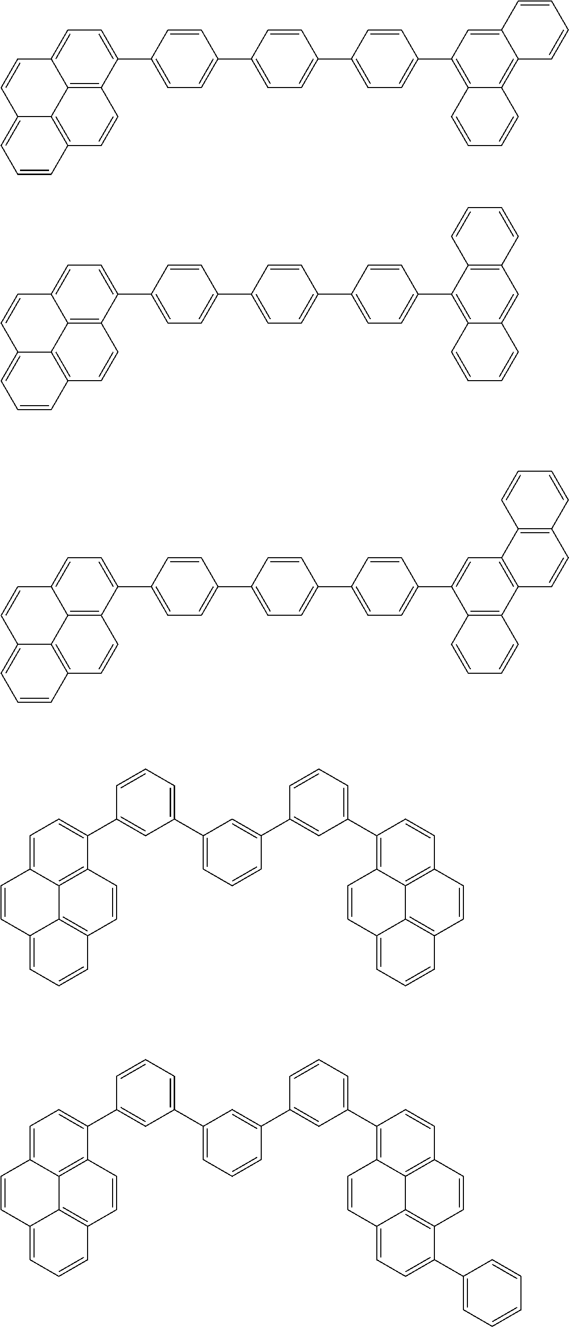

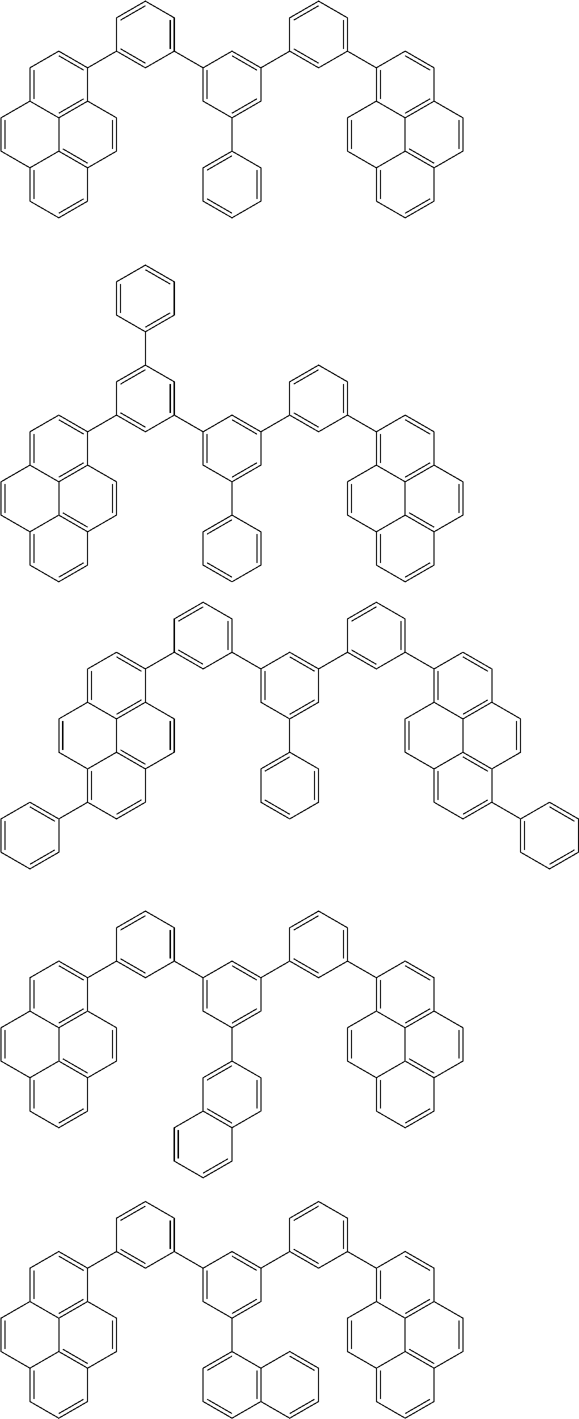

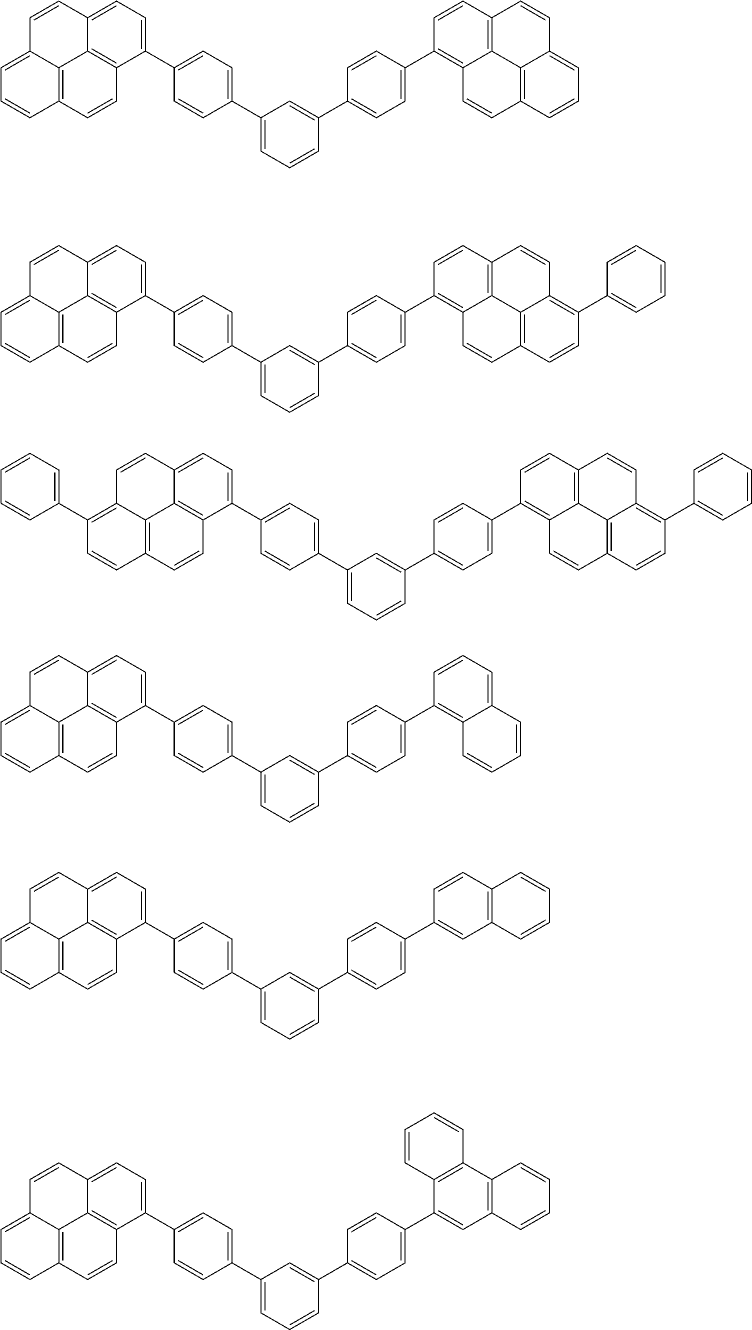

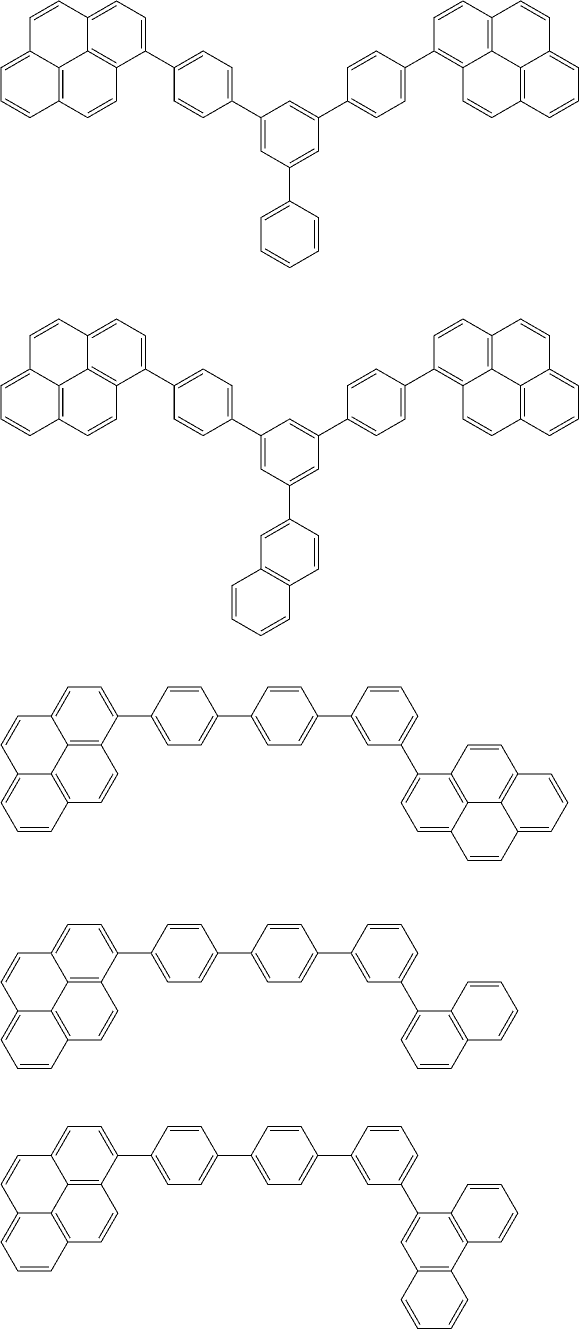

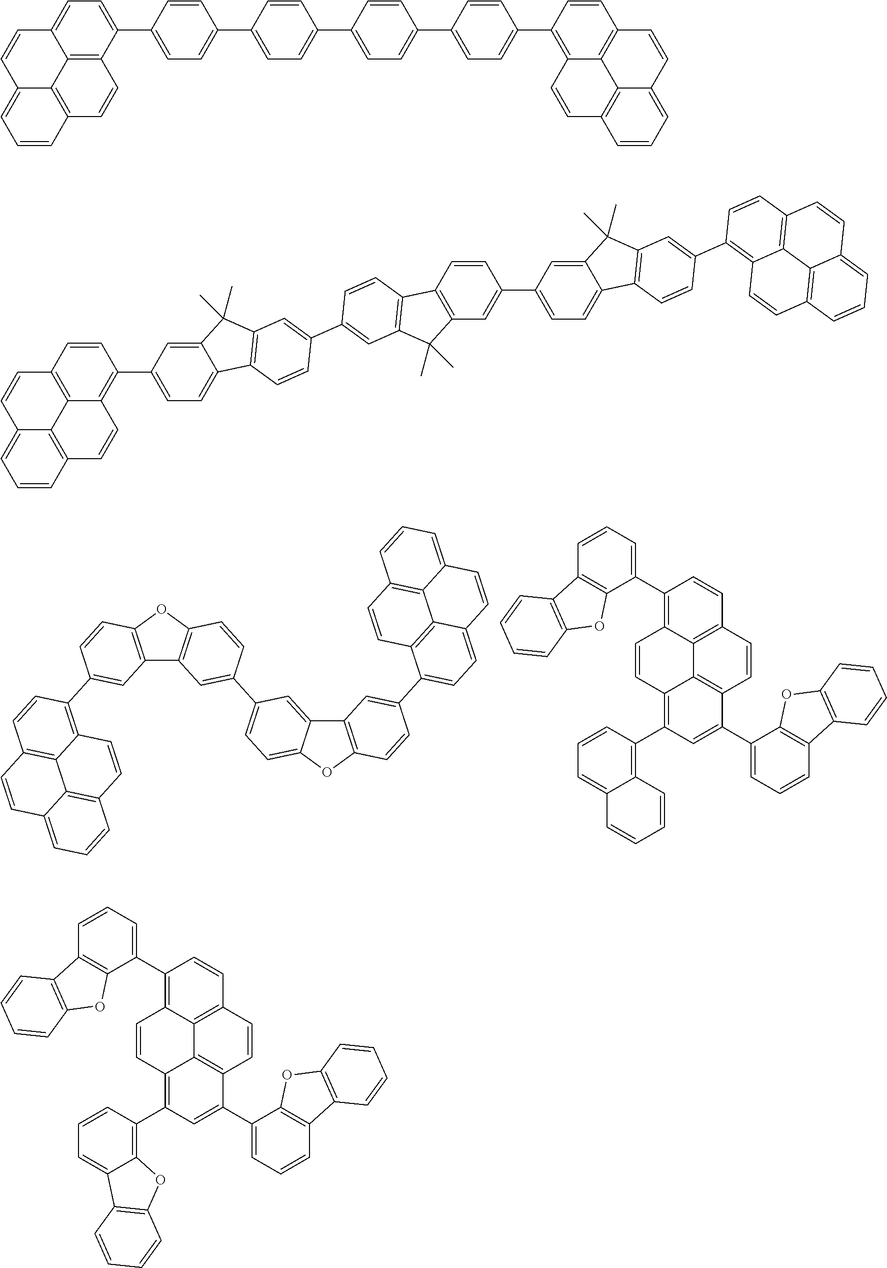

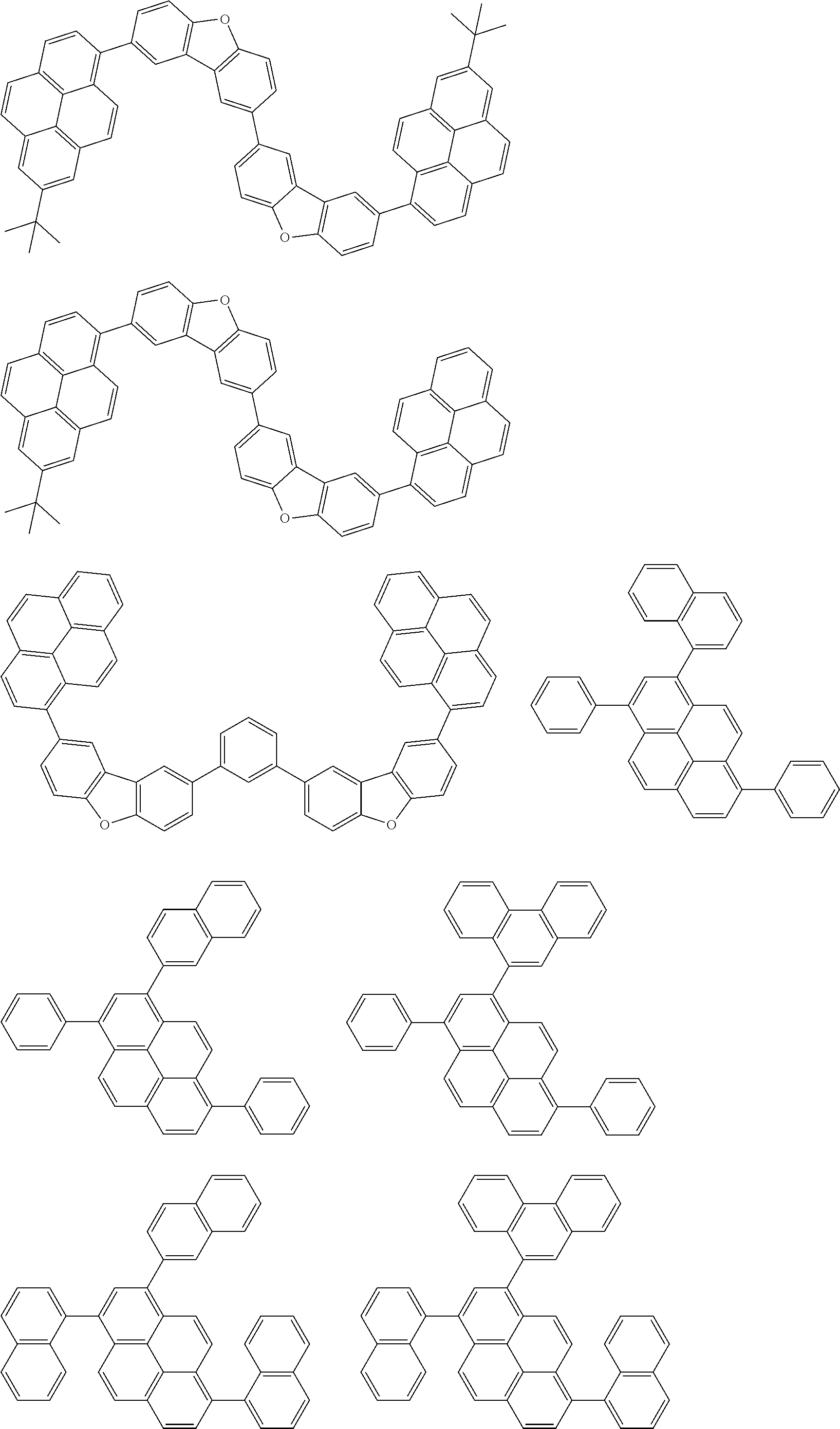

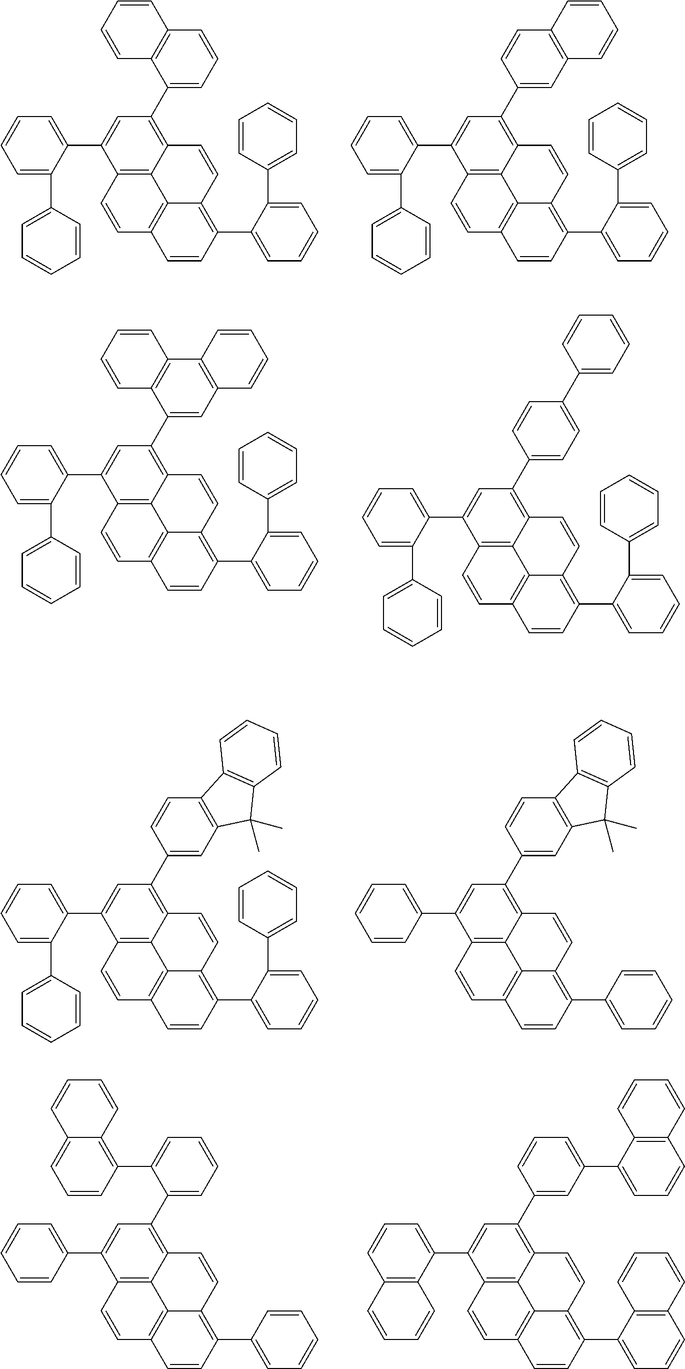

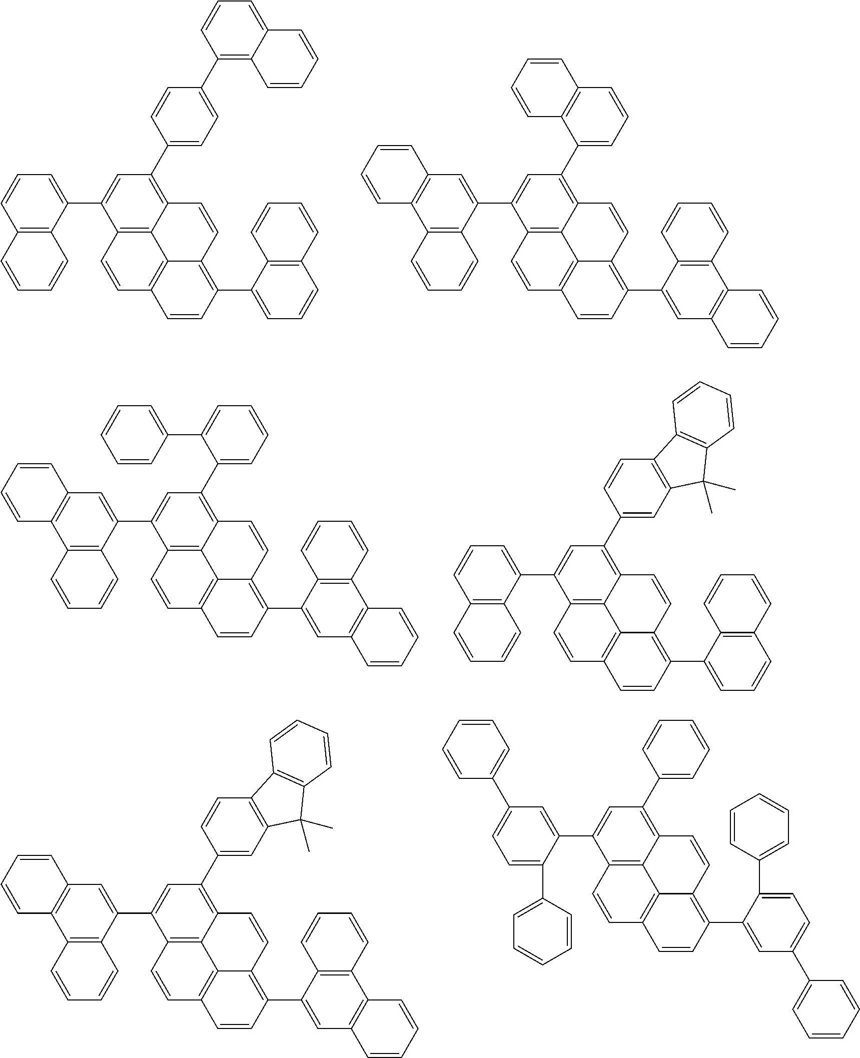

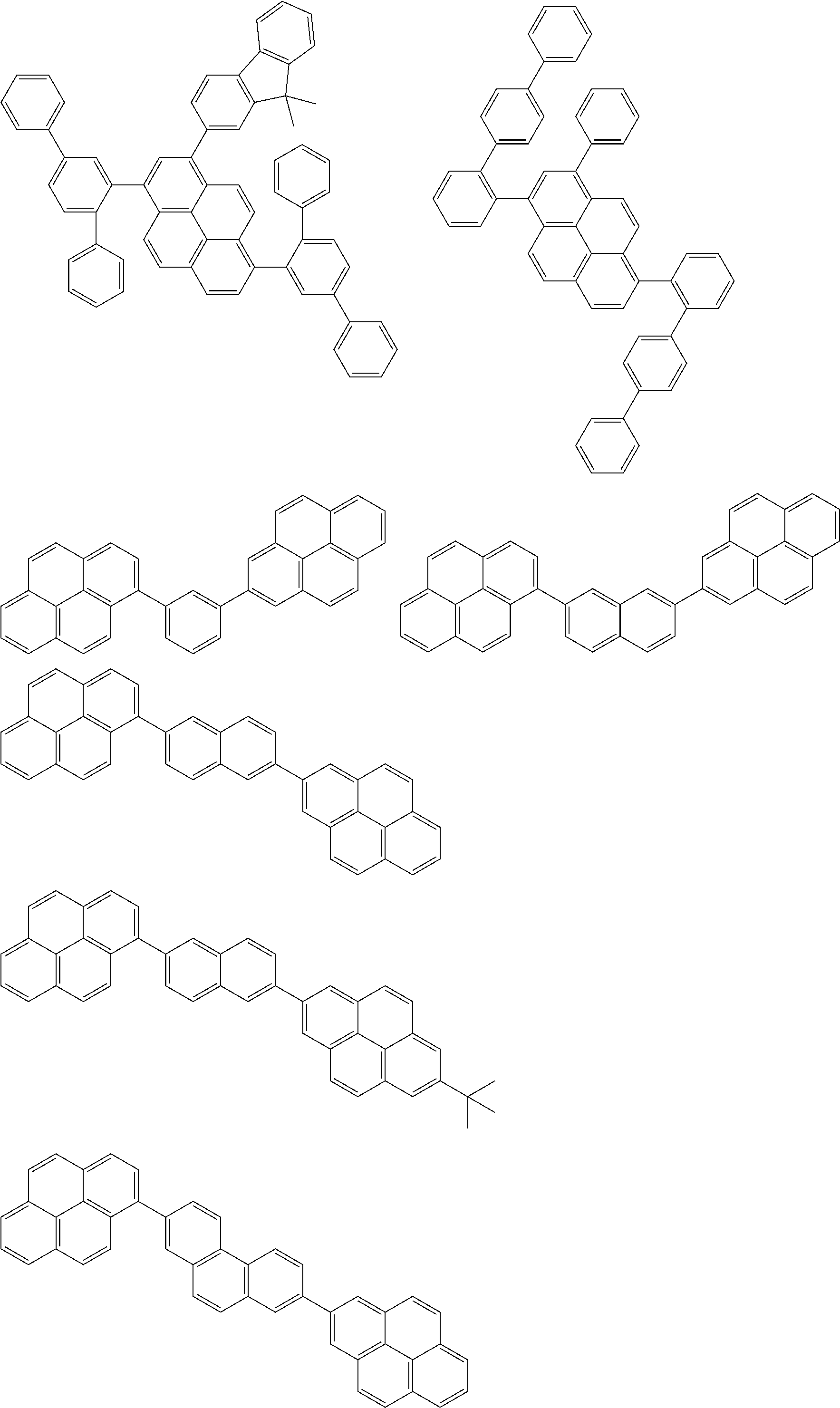

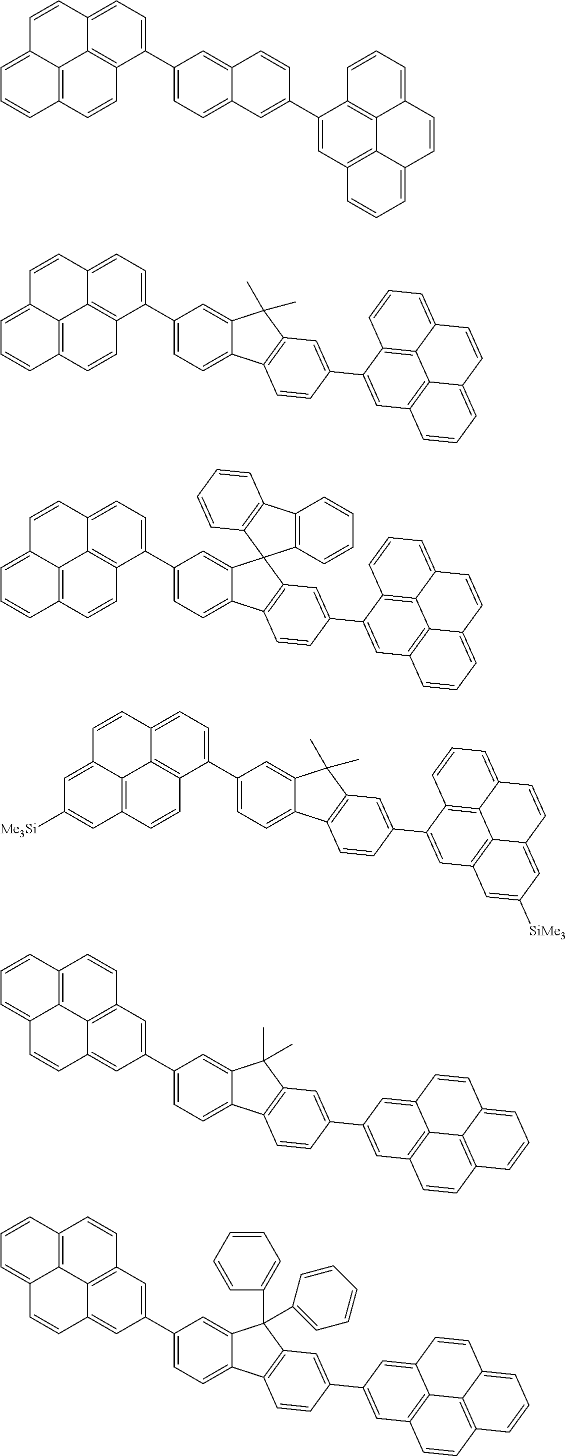

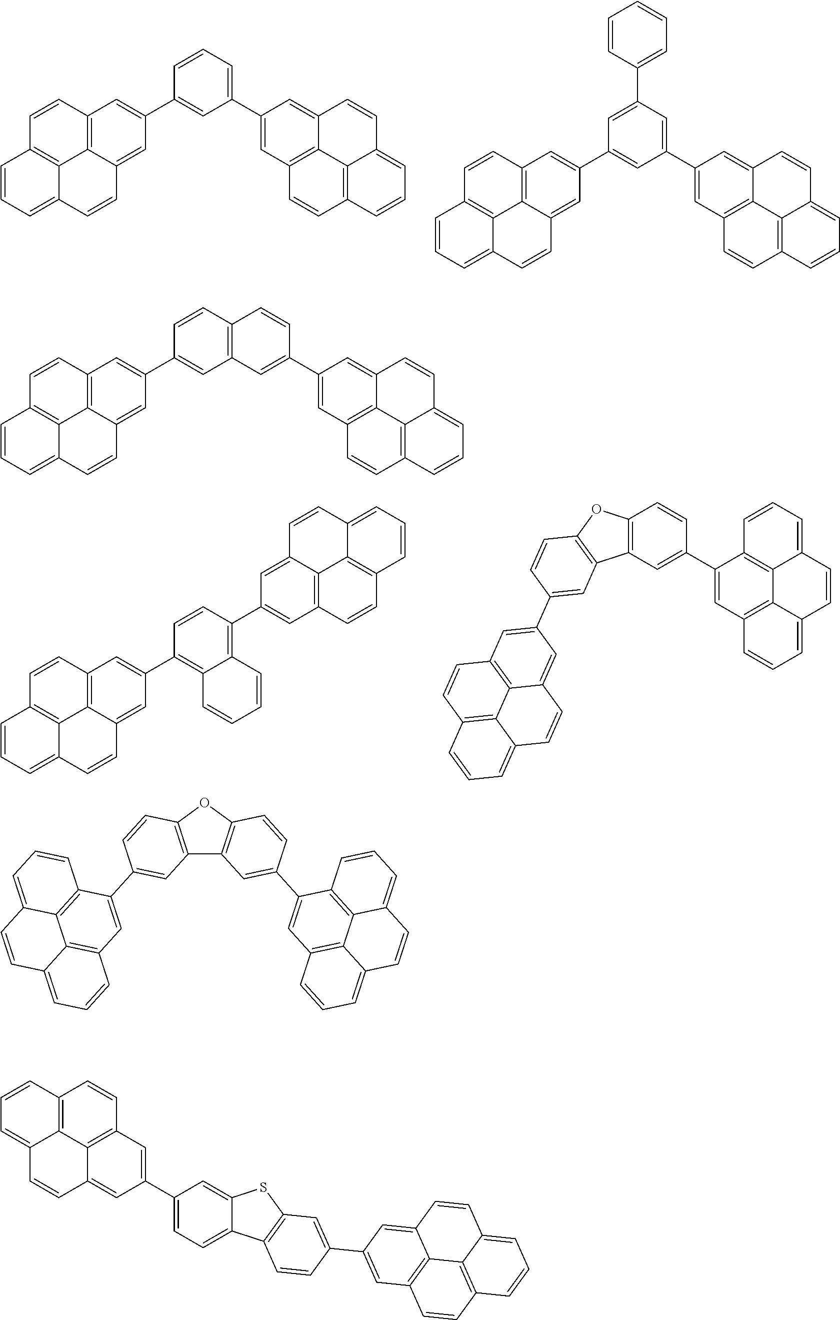

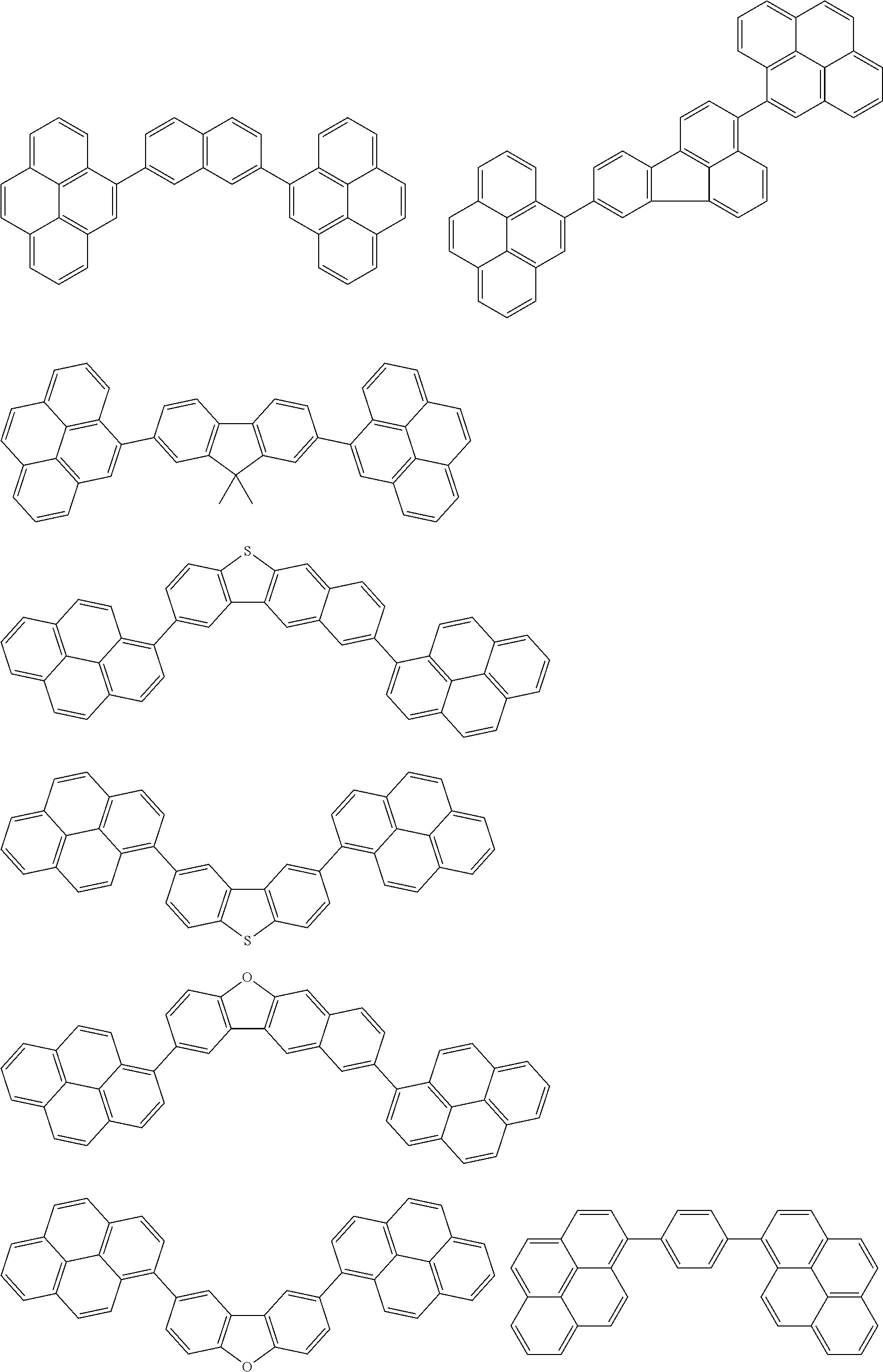

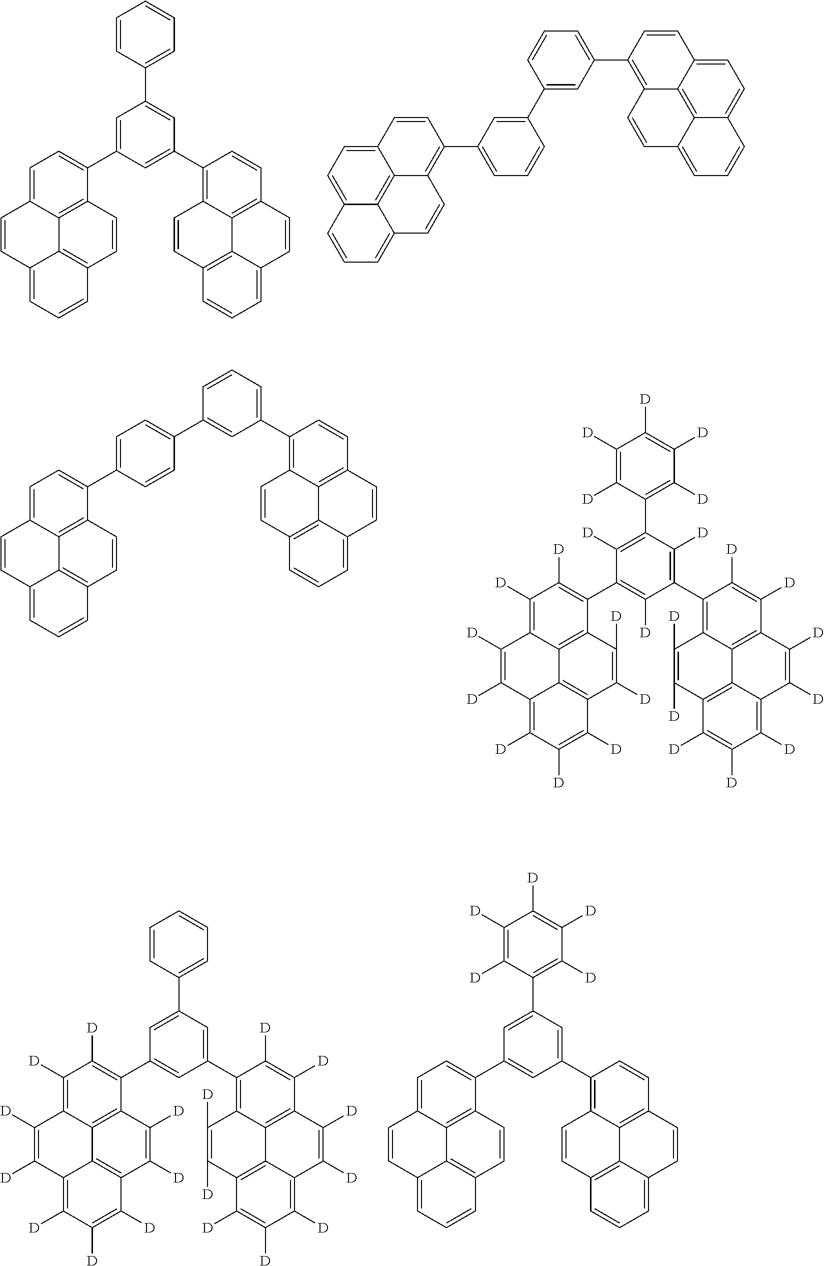

When the second host material is a benzanthracene derivative, the second host material is preferably a compound represented by the following general formula (1X).

(Compound represented by the general formula (1X))

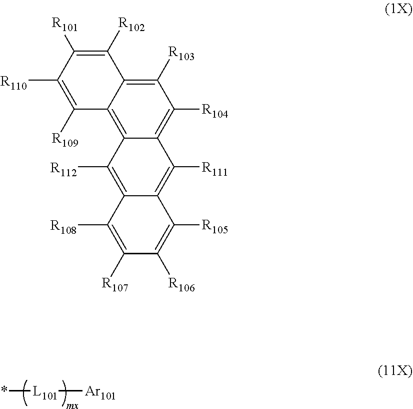

In the general formula (1X),
R₁₀₁ to R₁₁₂ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms,
a substituted or unsubstituted heterocyclic group having 5 to 50 ring atoms, or
the group represented by the general formula (11X);
provided that at least one of R₁₀₁ to R₁₁₂ is the group represented by the general formula (11X);
when a plurality of groups represented by the general formula (11X) is present, the plurality of groups represented by the general formula (11X) is the same as or different from each other;
L₁₀₁ is
a single bond,
a substituted or unsubstituted arylene group having 6 to 50 ring carbon atoms, or
a substituted or unsubstituted divalent heterocyclic group having 5 to 50 ring atoms;
Ar₁₀₁ is
a substituted or unsubstituted aryl group having 6 to 50 ring carbon atoms, or
a substituted or unsubstituted heterocyclic group having 5 to 50 ring atoms;
mx is 1, 2, 3, 4 or 5;
when two or more L₁₀₁'s are present, the two or more L₁₀₁'s are the same as or different from each other;
when two or more Ar₁₀₁'s are present, the two or more Ar₁₀₁'s are the same as or different from each other; and
* in the general formula (11X) represents a bonding position with the benz[a]anthracene ring in the general formula (1X).

In the organic EL device according to the present embodiment, the group represented by the general formula (11X) is preferably a group represented by the following general formula (111X).

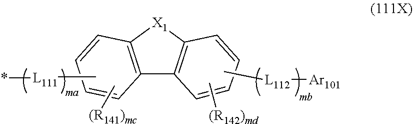

In the general formula (111X),
X1 is CR₁₄₃R₁₄₄, an oxygen atom, a sulfur atom, or NR₁₄₅;
L₁₁₁ and L₁₁₂ are independently
a single bond,
a substituted or unsubstituted arylene group having 6 to 50 ring carbon atoms, or
a substituted or unsubstituted divalent heterocyclic group having 5 to 50 ring atoms;
ma is 1, 2, 3 or 4;
mb is 1, 2, 3 or 4;
ma+mb is 2, 3 or 4;
Ar₁₀₁ is the same as defined for Ar₁₀₁ in the general formula (11X);
R₁₄₁, R₁₄₂, R₁₄₃, R₁₄₄ and R₁₄₅ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
mc is 3;
the three R₁₄₁'s are the same as or different from each other;
md is 3; and
the three R₁₄₂'s are the same as or different from each other.

L₁₁₁ is bonded with any one position of carbon atoms *1 to *4 in positions of carbon atoms *1 to *8 in a ring structure represented by the following general formula (111aX) of the group represented by the general formula (111X), R₁₄₁ is bonded with the remaining three positions of carbon atoms *1 to *4, L₁₁₂ is bonded with any one position of carbon atoms *5 to *8 and R₁₄₂ is bonded with the remaining three positions of carbon atoms *5 to *8.

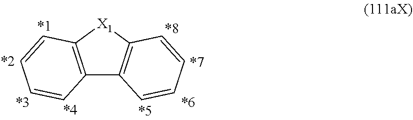

For example, in the group represented by the general formula (111X), when L₁₁₁ is bonded with the position of the carbon atom *2 in the ring structure represented by the general formula (111aX) and L₁₁₂ is bonded with the position of the carbon atom *7 in the ring structure represented by the general formula (111aX), the group represented by the general formula (111X) is represented by the following general formula (111bX).

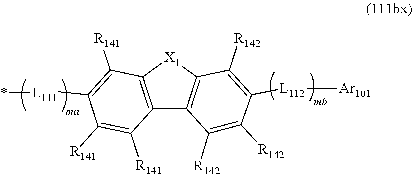

In the general formula (111bX),
X₁, L₁₁₁, L₁₁₂, ma, mb, Ar₁₀₁, R₁₄₁, R₁₄₂, R₁₄₃, R₁₄₄ and R₁₄₅ are independently the same as defined for X₁, L₁₁₁, L₁₁₂, ma, mb, Ar₁₀₁, R₁₄₁, R₁₄₂, R₁₄₃, R₁₄₄ and R₁₄₅ in the general formula (111X);
a plurality of R₁₄₁'s is the same as or different from each other; and
a plurality of R₁₄₂'s is the same as or different from each other.

In the organic EL device according to the present embodiment, the group represented by the general formula (111X) is preferably the group represented by the general formula (111bX).

In the compound represented by the general formula (1X), it is preferable that ma be 1 or 2, and mb be 1 or 2.

In the compound represented by the general formula (1X), it is preferable that ma be 1, and mb be 1.

In the compound represented by the general formula (1X), Ar₁₀₁ is preferably a substituted or unsubstituted aryl group having 6 to 50 ring carbon atoms.

In the compound represented by the general formula (1X), Ar₁₀₁ is preferably
a substituted or unsubstituted phenyl group,
a substituted or unsubstituted naphthyl group,
a substituted or unsubstituted biphenyl group,
a substituted or unsubstituted terphenyl group,
a substituted or unsubstituted benz[a]anthryl group,
a substituted or unsubstituted pyrenyl group,
a substituted or unsubstituted phenanthryl group, or
a substituted or unsubstituted fluorenyl group.

The compound represented by the general formula (1X) is also preferably represented by the following general formula (101X).

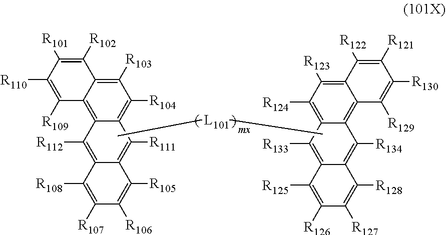

In the general formula (101X),
one of R₁₁₁ and R₁₁₂ is represents a bonding position with L₁₀₁, and one of R₁₃₃ and R₁₃₄ is represents a bonding position with L₁₀₁;
R₁₀₁ to R₁₁₀₁R₁₂₁ to R₁₃₀, R₁₁₁ or R₁₁₂ which is not the bonding position with L₁₀₁, and R₁₃₃ or R₁₃₄ which is not the bonding position with L₁₀₁ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
L₁₀₁ is
a single bond,
a substituted or unsubstituted arylene group having 6 to 50 ring carbon atoms, or
a substituted or unsubstituted divalent heterocyclic group having 5 to 50 ring atoms;
mx is 1, 2, 3, 4 or 5; and
when two or more L₁₀₁'s are present, the two or more L₁₀₁'s are the same as or different from each other.

In the compound represented by the general formula (1X), L₁₀₁ is preferably a single bond, or a substituted or unsubstituted arylene group having 6 to 50 ring carbon atoms.

The compound represented by the general formula (1X) is also preferably represented by the following general formula (102X).

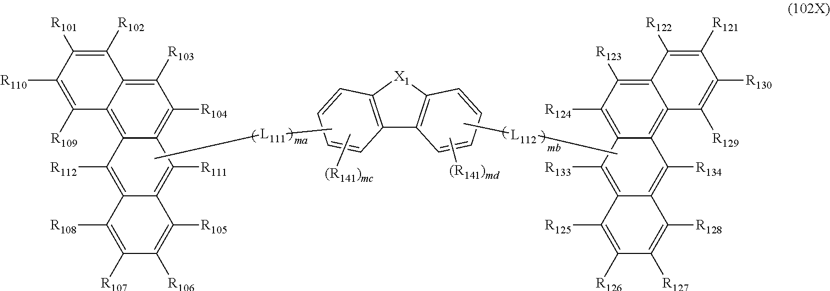

In the general formula (102X),
one of R₁₁₁ and R₁₁₂ is represents a bonding position with L₁₁₁, and one of R₁₃₃ and R₁₃₄ is represents a bonding position with 111₂;
R₁₀₁ to R₁₁₀, R₁₂₁ to R₁₃₀, R₁₁₁ or R₁₁₂ which is not the bonding position with L₁₁₁, and R₁₃₃ or R₁₃₄ which is not the bonding position with —L₁₁₂ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
X₁ is CR₁₄₃R₁₄₄, an oxygen atom, a sulfur atom, or NR₁₄₅;
L₁₁₁ and L₁₁₂ are independently
a single bond,
a substituted or unsubstituted arylene group having 6 to 50 ring carbon atoms, or
a substituted or unsubstituted divalent heterocyclic group having 5 to 50 ring atoms;
ma is 1, 2, 3 or 4;
mb is 1, 2, 3 or 4;
ma+mb is 2, 3, 4 or 5;
R₁₄₁, R₁₄₂, R₁₄₃, R₁₄₄ and R₁₄₅ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
mc is 3;
the three R₁₄₁'s are the same as or different from each other;
md is 3; and
the three R₁₄₂'s are the same as or different from each other.

In the compound represented by the general formula (1X), it is preferable that ma in the general formula (102X) be 1 or 2, and mb be 1 or 2.

In the compound represented by the general formula (1X), it is preferable that ma in the general formula (102X) be 1, and mb be 1.

In the compound represented by the general formula (1X), the group represented by the general formula (11X) is also preferably a group represented by the following general formula (11AX), or a group represented by the following general formula (11 BX).

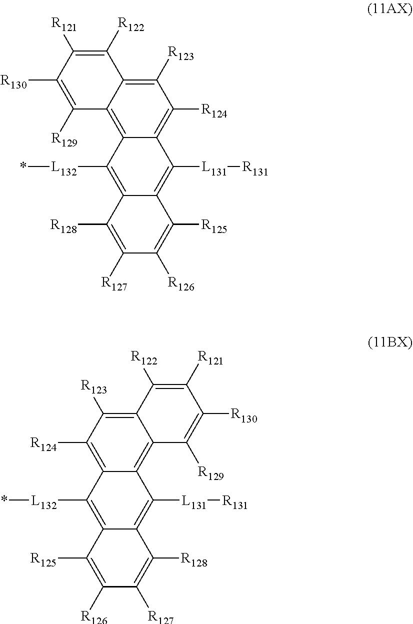

In the general formula (11AX) and the general formula (11BX),
R₁₂₁ to R₁₃₁ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms, or
a substituted or unsubstituted heterocyclic group having 5 to 50 ring atoms;
when a plurality of groups represented by the general formula (11AX) is present, the plurality of groups represented by the general formula (11AX) is the same as or different from each other;
when a plurality of groups represented by the general formula (11 BX) is present, the plurality of groups represented by the general formula (11 BX) is the same as or different from each other;
L₁₃₁ and L₁₃₂ are independently
a single bond,
a substituted or unsubstituted arylene group having 6 to 50 ring carbon atoms, or
a substituted or unsubstituted divalent heterocyclic group having 5 to 50 ring atoms; and
each of *'s in the general formula (11AX) and in the general formula (11BX) represents a bonding position with the benz[a]anthracene ring in the general formula (1X).

The compound represented by the general formula (1X) is also preferably represented by the following general formula (103X).

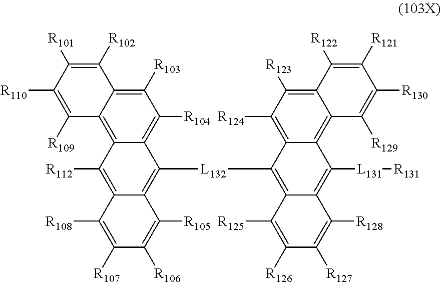

In the general formula (103X),
R₁₀₁ to R₁₁₀ and R₁₁₂ are independently the same as defined for R₁₀₁ to R₁₁₀ and R₁₁₂ in the general formula (1X); and
R₁₂₁ to R₁₃₁, L₁₃₁ and L₁₃₂ are independently the same as defined for R₁₂₁ to R₁₃₁, L₁₃₁ and L₁₃₂ in the general formula (11BX).

In the compound represented by the general formula (1X), L₁₃₁ is also preferably a substituted or unsubstituted arylene group having 6 to 50 ring carbon atoms.

In the compound represented by the general formula (1X), L₁₃₂ is also preferably a substituted or unsubstituted arylene group having 6 to 50 ring carbon atoms.

In the compound represented by the general formula (1X), two or more of R₁₀₁ to R₁₁₂ are also preferably the group represented by the general formula (11).

In the compound represented by the general formula (1X), it is preferable that two or more of R₁₀₁ to R₁₁₂ be the group represented by the general formula (11X), and Ar₁₀₁ in the general formula (11X) is a substituted or unsubstituted aryl group having 6 to 50 ring carbon atoms.

In the compound represented by the general formula (1X),
it is also preferable that Ar₁₀₁ be not a substituted or unsubstituted benz[a]anthryl group,
L₁₀₁ be not a substituted or unsubstituted benz[a]anthrylene group, and
a substituted or unsubstituted aryl group having 6 to 50 ring carbon atoms for R₁₀₁ to R₁₁₀ which are not the group represented by the general formula (11X) be not a substituted or unsubstituted benz[a]anthryl group.

In the compound represented by the general formula (1X), it is preferable that R₁₀₁ to R₁₁₂ which are not the group represented by the general formula (11X) be independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the compound represented by the general formula (1X), it is preferable that R₁₀₁ to R₁₁₂ which are not the group represented by the general formula (11X) be independently a hydrogen atom, a substituted or unsubstituted alkyl group having 1 to 50 carbon atoms, or a substituted or unsubstituted cycloalkyl group having 3 to 50 ring carbon atoms.

In the compound represented by the general formula (1X), R₁₀₁ to R₁₁₂ which are not the group represented by the general formula (11X) are preferably hydrogen atoms.

The compound represented by the general formula (1X) can be produced by a known method.

### (Specific Examples of Compound Represented by the General Formula (1X))

Specific examples of the compound represented by the general formula (1X) include the following compounds. Here, the compound represented by the general formula (1X) is not limited to the following specific examples.

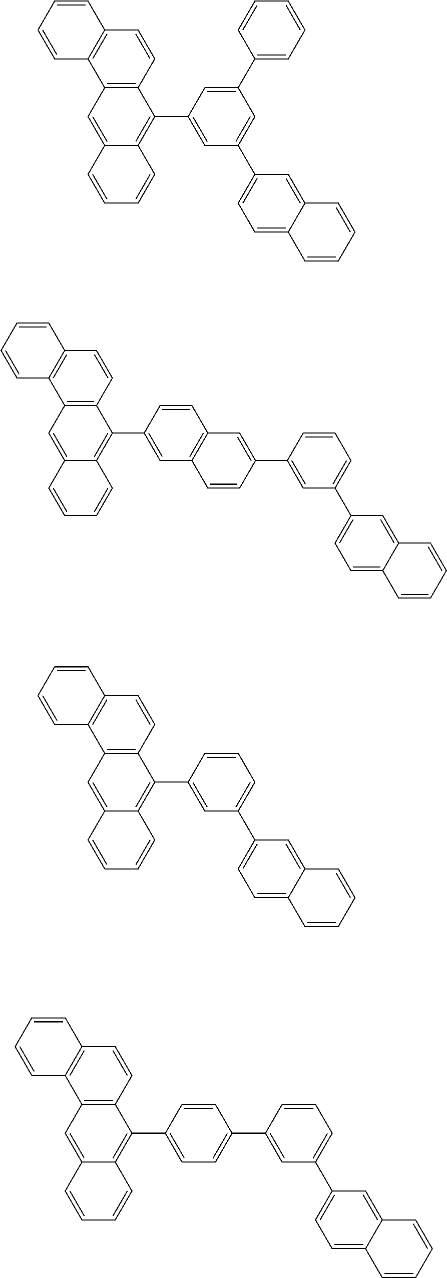

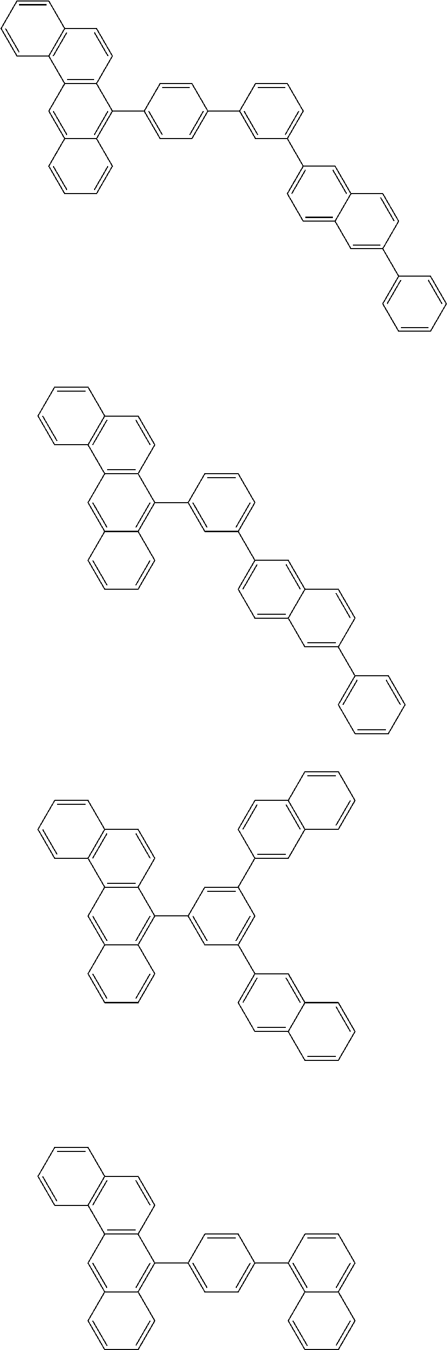

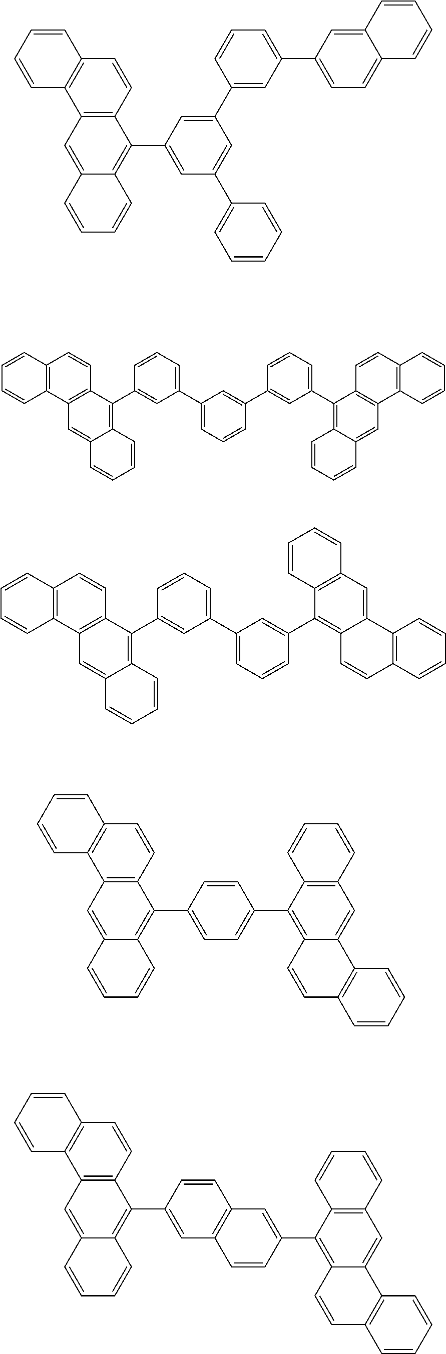

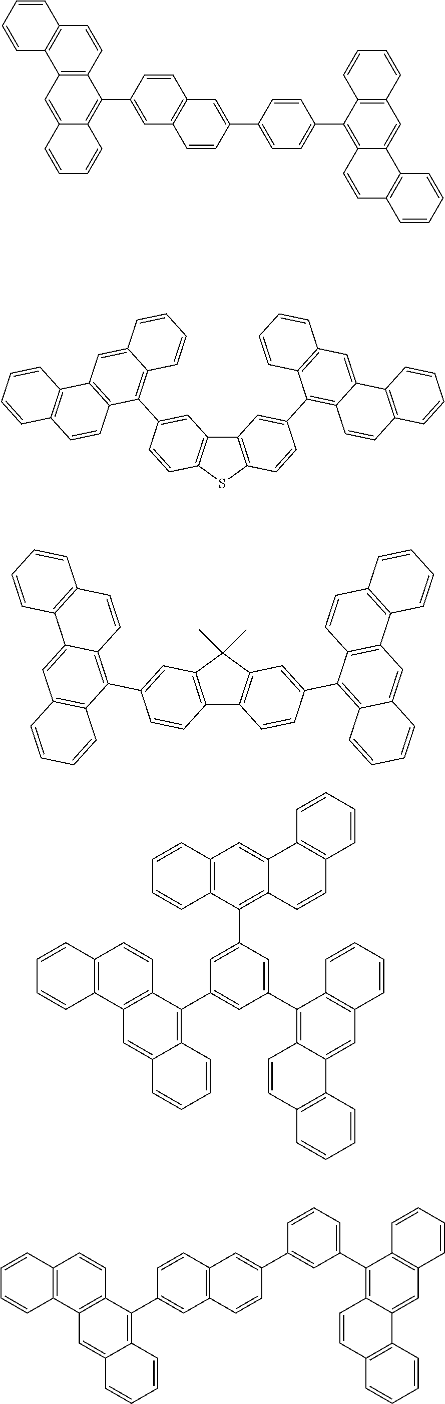

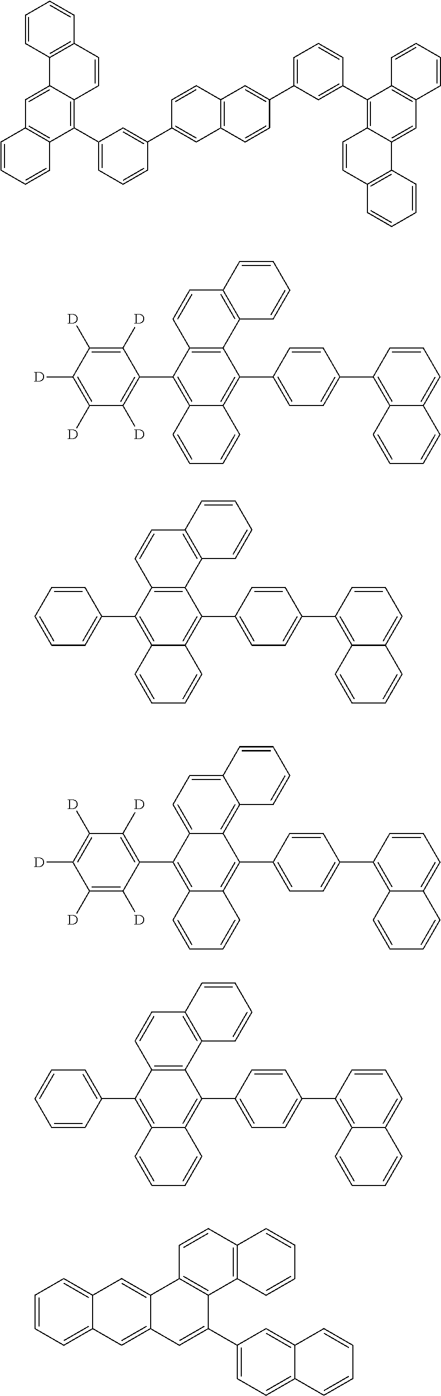

When the second host material is a benzoxanthene derivative, the second host material is preferably a compound represented by the following general formula (14X).

### (Compound Represented by the General Formula (14X))

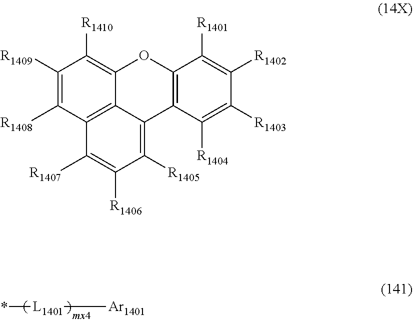

In the general formula (14X),
R₁₄₀₁ to R₁₄₁₀ are independently
a hydrogen atom,
a substituted or unsubstituted alkyl group having 1 to 50 carbon atoms,
a substituted or unsubstituted haloalkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a substituted or unsubstituted aralkyl group having 7 to 50 carbon atoms,
a group represented by —C(═O)R₈₀₁,
a group represented by —COOR₈₀₂,
a halogen atom,
a cyano group,
a nitro group,
a substituted or unsubstituted aryl group having 6 to 50 ring carbon atoms,
a substituted or unsubstituted heterocyclic group having 5 to 50 ring atoms, or
the group represented by the general formula (141);
provided that at least one of R₁₄₀₁ to R₁₄₁₀ is the group represented by the general formula (141);
when a plurality of groups represented by the general formula (141) is present, the plurality of groups represented by the general formula (141) is the same as or different from each other;
L₁₄₀₁ is
a single bond,
a substituted or unsubstituted arylene group having 6 to 50 ring carbon atoms, or
a substituted or unsubstituted divalent heterocyclic group having 5 to 50 ring atoms;
Ar₁₄O₁ is
a substituted or unsubstituted aryl group having 6 to 50 ring carbon atoms, or
a substituted or unsubstituted heterocyclic group having 5 to 50 ring atoms;
mx4 is 0, 1, 2, 3, 4 or 5;
when two or more L₁₄₀₁'s are present, the two or more L₁₄₀₁'s are the same as or different from each other;
when two or more Ar₁₄₀₁'s are present, the two or more Ar₁₄₀₁'s are the same as or different from each other; and
* in the general formula (141) represents a bonding position with the ring represented by the general formula (14X).

The compound represented by the general formula (14X) can be produced by a known method.

### (Specific Examples of Compound Represented by the General Formula (14X))

Specific examples of the compound represented by the general formula (14X) include the following compounds. Here, the compound represented by the general formula (14X) is not limited to the following specific examples.

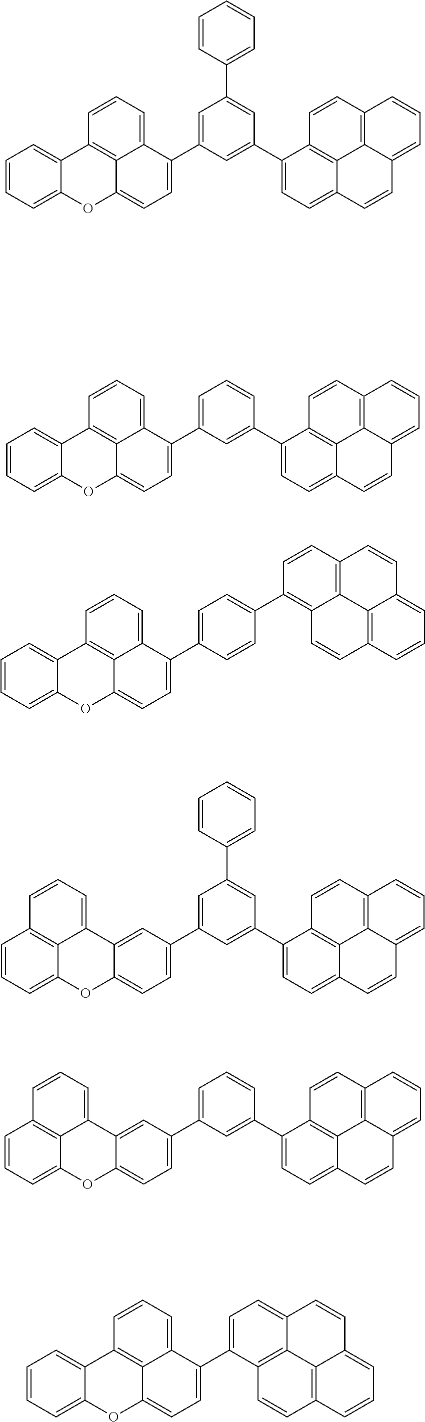

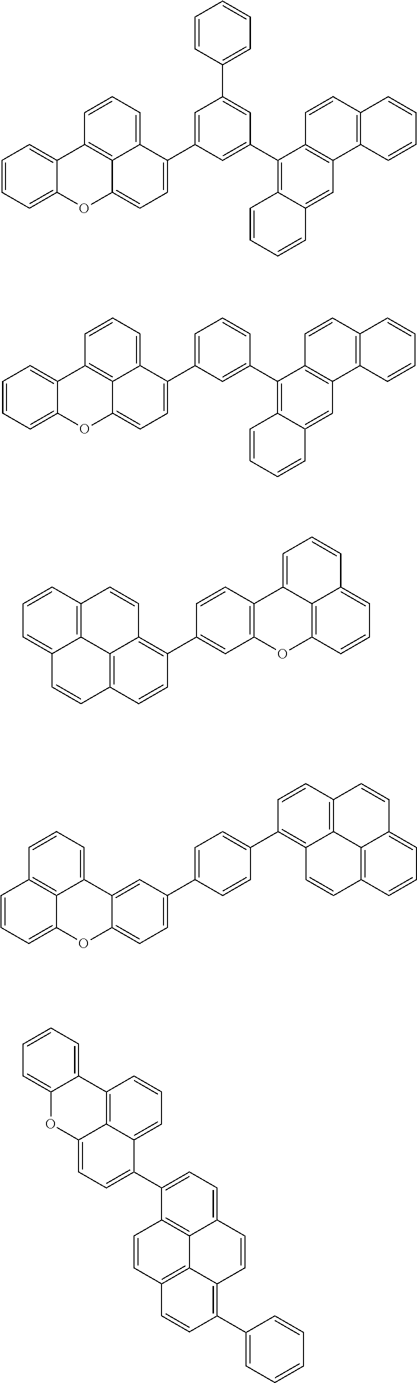

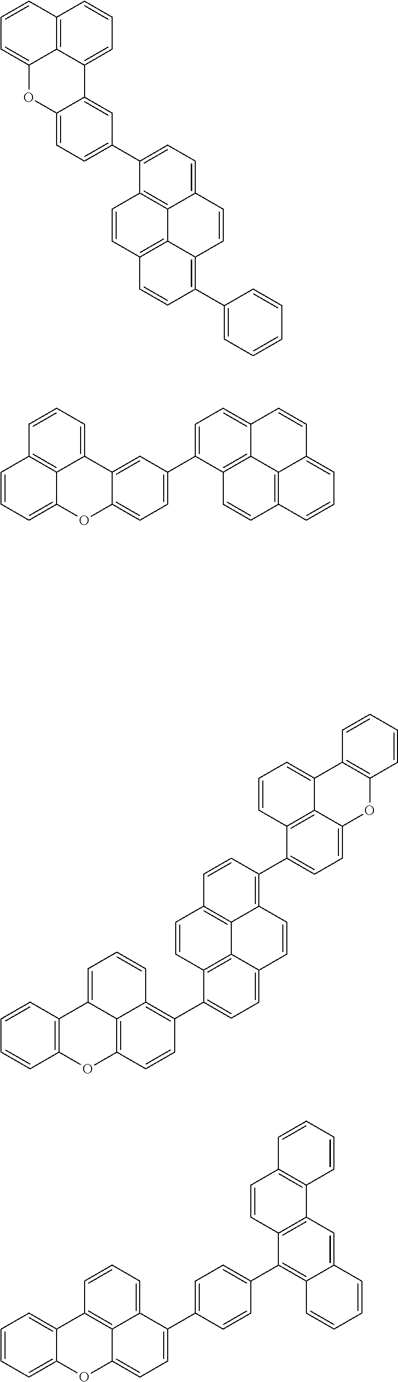

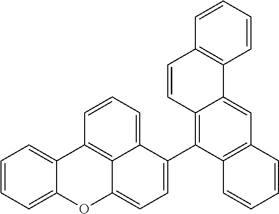

### (Second Dopant Material)

Examples of a second dopant material include the first compound represented by the formula (1), pyrene derivatives, styrylamine derivatives, chrysene derivatives, fluoranthene derivatives, fluorene derivatives, diamine derivatives, triarylamine derivatives, aromatic amine derivatives, and tetracene derivatives.

The second dopant material is preferably the first compound represented by the formula (1), a compound represented by the following general formula (5), or a compound represented by the following general formula (6).

### (Compound Represented by the General Formula (5))

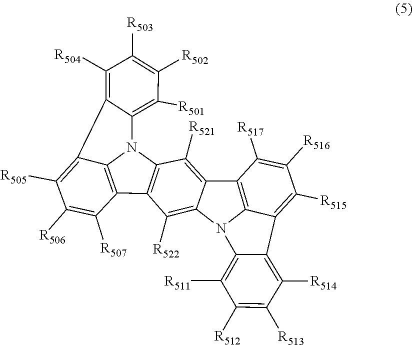

In the general formula (5),
one or more sets of the adjacent two or more of R₅₀₁ to R₅₀₇ and R₅₁₁ to R₅₁₇
form a substituted or unsubstituted single ring by bonding with each other,
form a substituted or unsubstituted fused ring by bonding with each other, or
do not bond with each other;
R₅₀₁ to R₅₀₇ and R₅₁₁ to R₅₁₇ which do not form the single ring and which do not form the fused ring are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a group represented by —N(R₉₀₆)(R₉₀₇),
a halogen atom,
a cyano group,
a nitro group,
a substituted or unsubstituted aryl group having 6 to 50 ring carbon atoms, or
a substituted or unsubstituted heterocyclic group having 5 to 50 ring atoms; and
R₅₂₁ and R₅₂₂ are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a group represented by —N(R₉₀₆)(R₉₀₇),
a halogen atom,
a cyano group,
a nitro group,
a substituted or unsubstituted aryl group having 6 to 50 ring carbon atoms, or
a substituted or unsubstituted heterocyclic group having 5 to 50 ring atoms.

The expression “one set of the adjacent two or more of R₅₀₁ to R₅₀₇ and R₅₁₁ to R₅₁₇” is, for example, a combination such as a set of R₆₀ₛ and R₅₀₂, a set of R₅₀₂ and R₅₀₃, a set of R₅₀₃ and R₅₀₄, a set of R₆₀ₛ and R₉₀₅, a set of R₆₀ₛ and R₅₀₇ and a set of R₅₀₁, R₅₀₂ and R₅₀₃.

In one embodiment, at least one of, preferably two of, R₅₀₁ to R₅₀₇ and R₅₁₁ to R₅₁₇ is a group represented by —N(R₉₀₅)(R₉₀₇).

In one embodiment, R₅₀₁ to R₅₀₇ and R₅₁₁ to R₅₁₇ are independently
a hydrogen atom,
a substituted or unsubstituted aryl group having 6 to 50 ring carbon atoms, or
a substituted or unsubstituted heterocyclic group having 5 to 50 ring atoms.

In one embodiment, the compound represented by the general formula (5) is a compound represented by the following general formula (52).

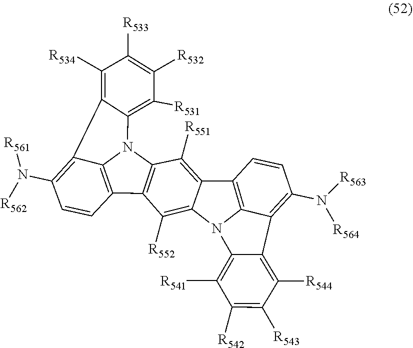

In the general formula (52),
one or more sets of the adjacent two or more of R₅₃₁ to R₅₃₄ and R₅₄₁ to R₅₄₄
form a substituted or unsubstituted single ring by bonding with each other,
form a substituted or unsubstituted fused ring by bonding with each other, or
do not bond with each other;
R₅₃₁ to R₅₃₄ and R₅₄₁ to R₅₄₄ which do not form the single ring and which do not form the fused ring, and R₅₅₁ and R₅₅₂ are independently
a hydrogen atom,
a substituted or unsubstituted aryl group having 6 to 50 ring carbon atoms, or
a substituted or unsubstituted heterocyclic group having 5 to 50 ring atoms;
R₅₆₁ to R₅₆₄ are independently
a substituted or unsubstituted aryl group having 6 to 50 ring carbon atoms, or
a substituted or unsubstituted heterocyclic group having 5 to 50 ring atoms.

In one embodiment, the compound represented by the general formula (5) is a compound represented by the following general formula (53).

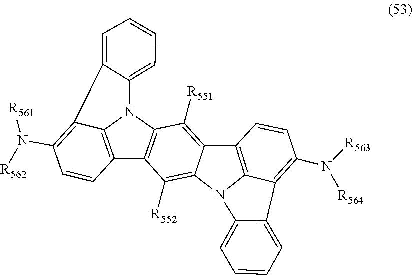

In the general formula (53), R₅₅₁, R₅₅₂ and R₅₆₁ to R₅₆₄ are independently the same as defined for R₅₅₁, R₅₅₂ and R₅₆₁ to R₅₉₄ in the general formula (52).

In one embodiment, R₅₆₁ to R₅₉₄ in the general formula (52) and the general formula (53) are independently a substituted or unsubstituted aryl group having 6 to 50 ring carbon atoms (preferably a phenyl group).

In one embodiment, R₅₂₁ and R₅₂₂ in the general formula (5), and R₅₅₂ and R₅₅₂ in the general formula (52) and the general formula (53) are hydrogen atoms.

In one embodiment, a substituent in the case of “substituted or unsubstituted” in the general formula (5), the general formula (52) and the general formula (53) is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms.

The compound represented by the general formula (5) can be produced by a known method.

### (Specific Examples of Compound Represented by the General Formula (5))

Specific examples of the compound represented by the general formula (5) include the following compounds. Here, the compound represented by the general formula (5) is not limited to

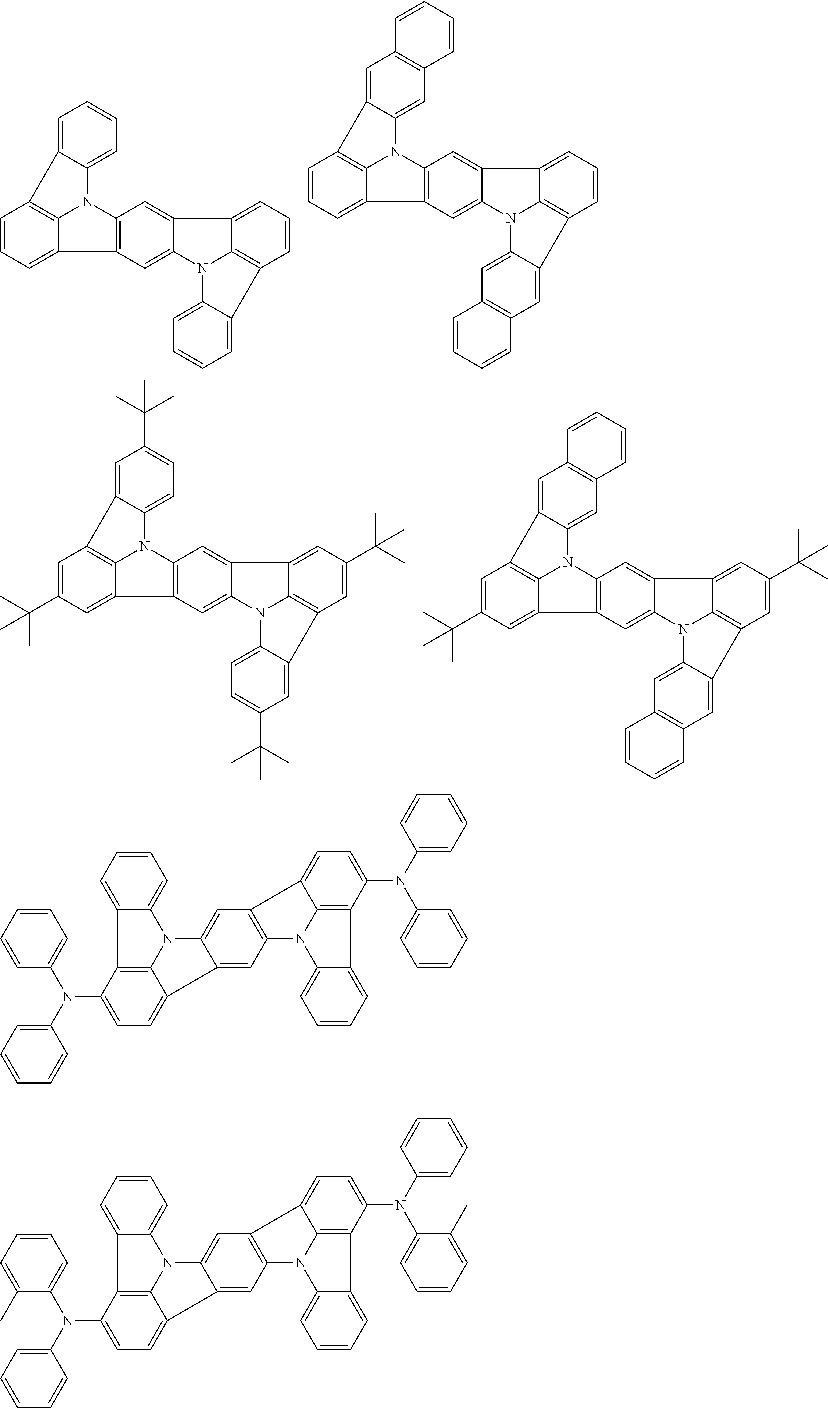

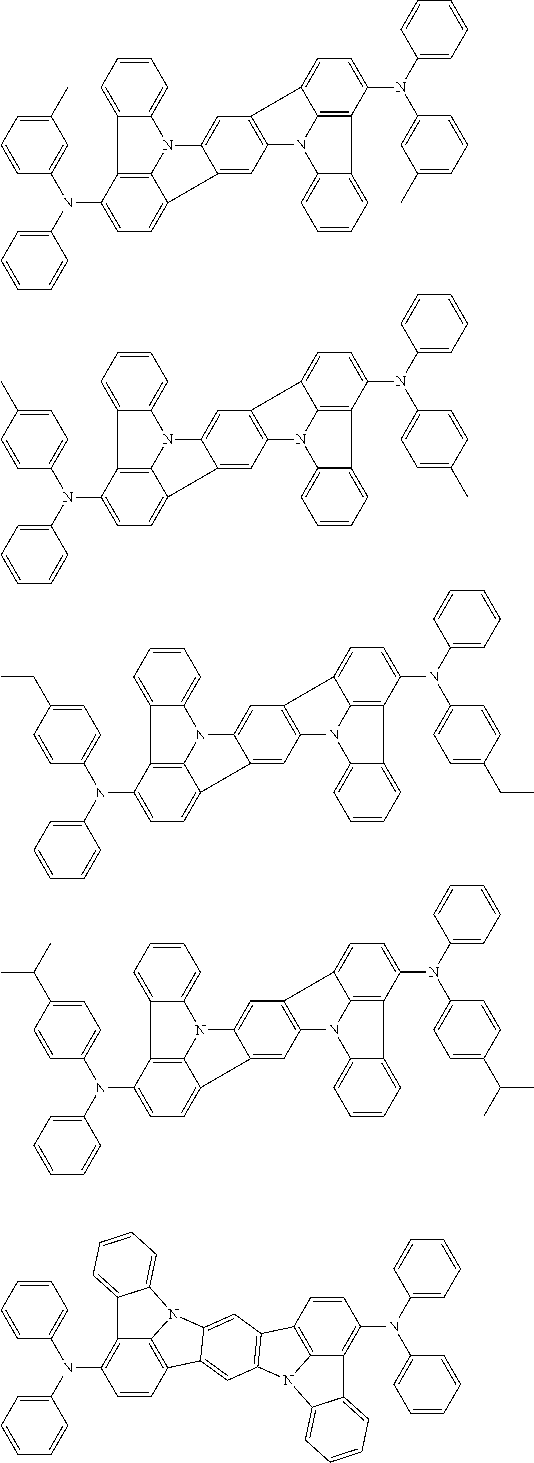

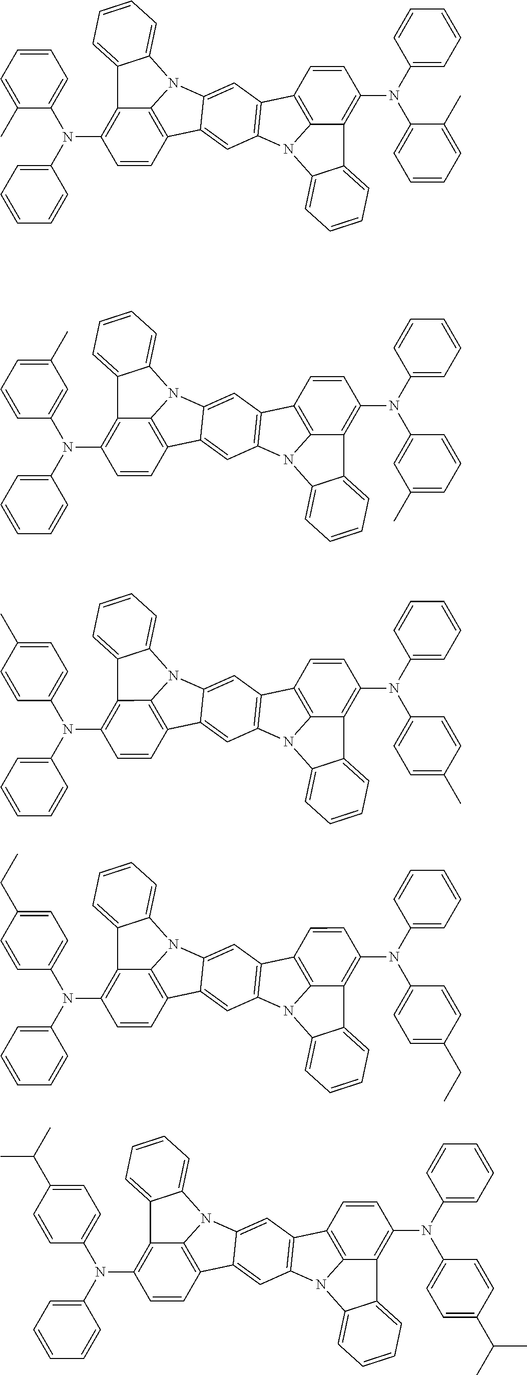

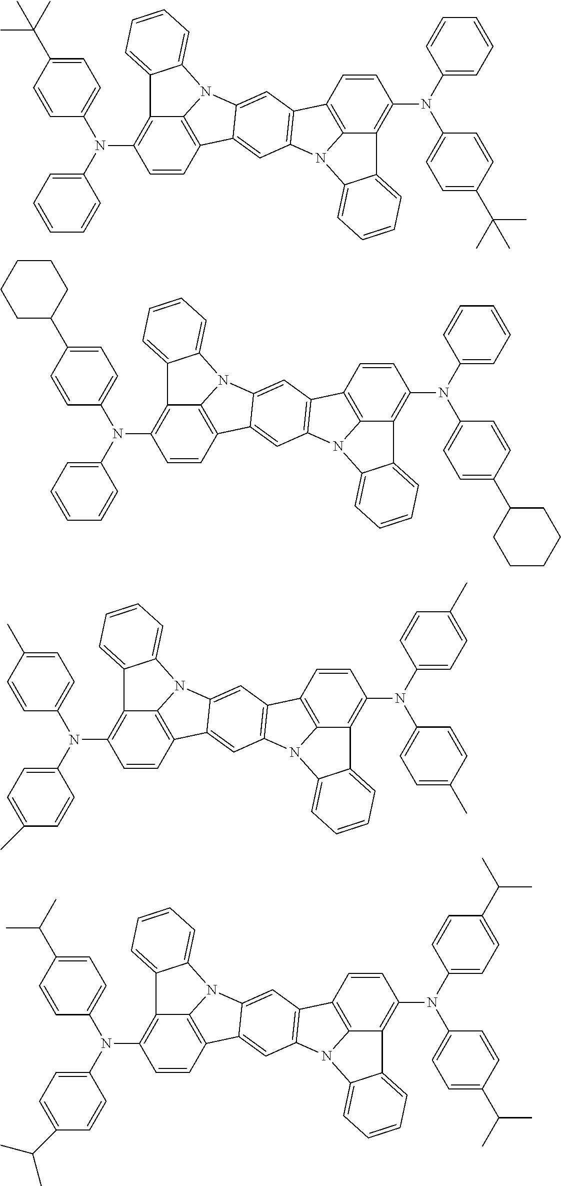

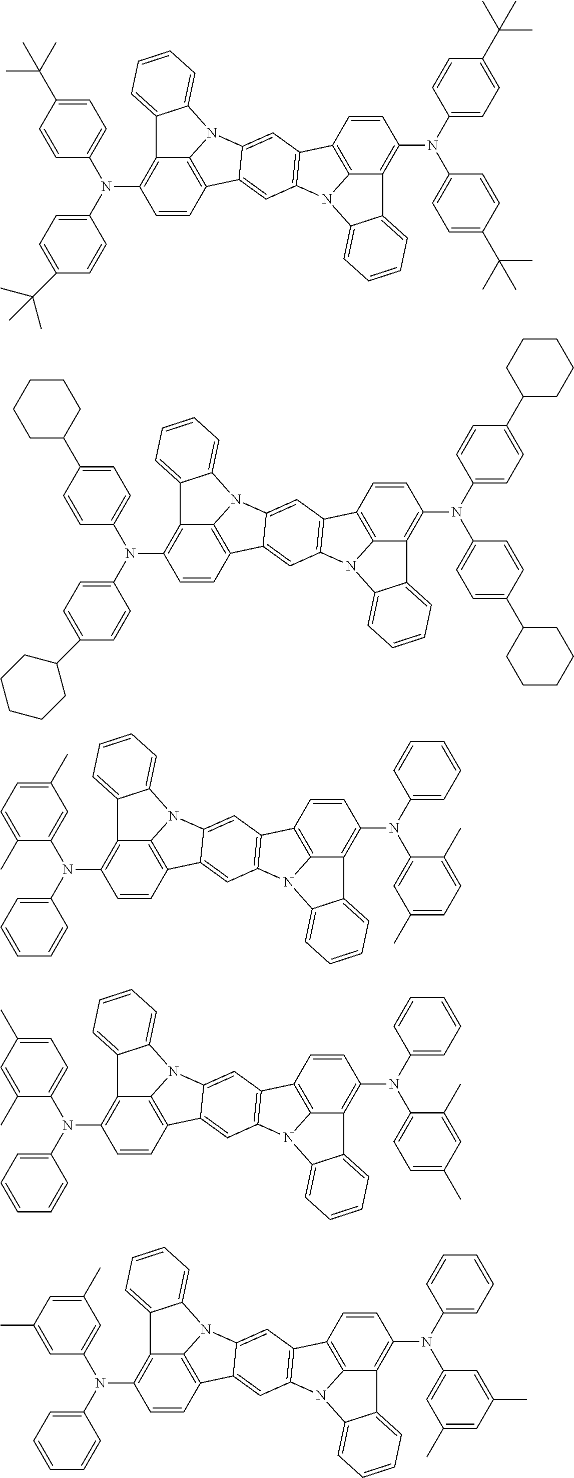

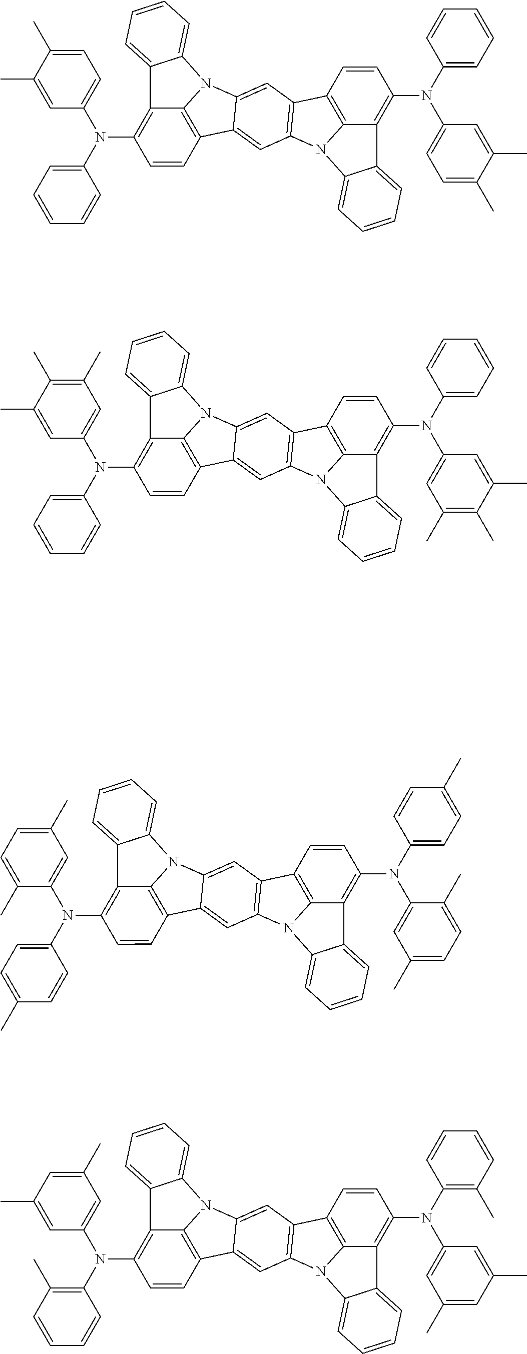

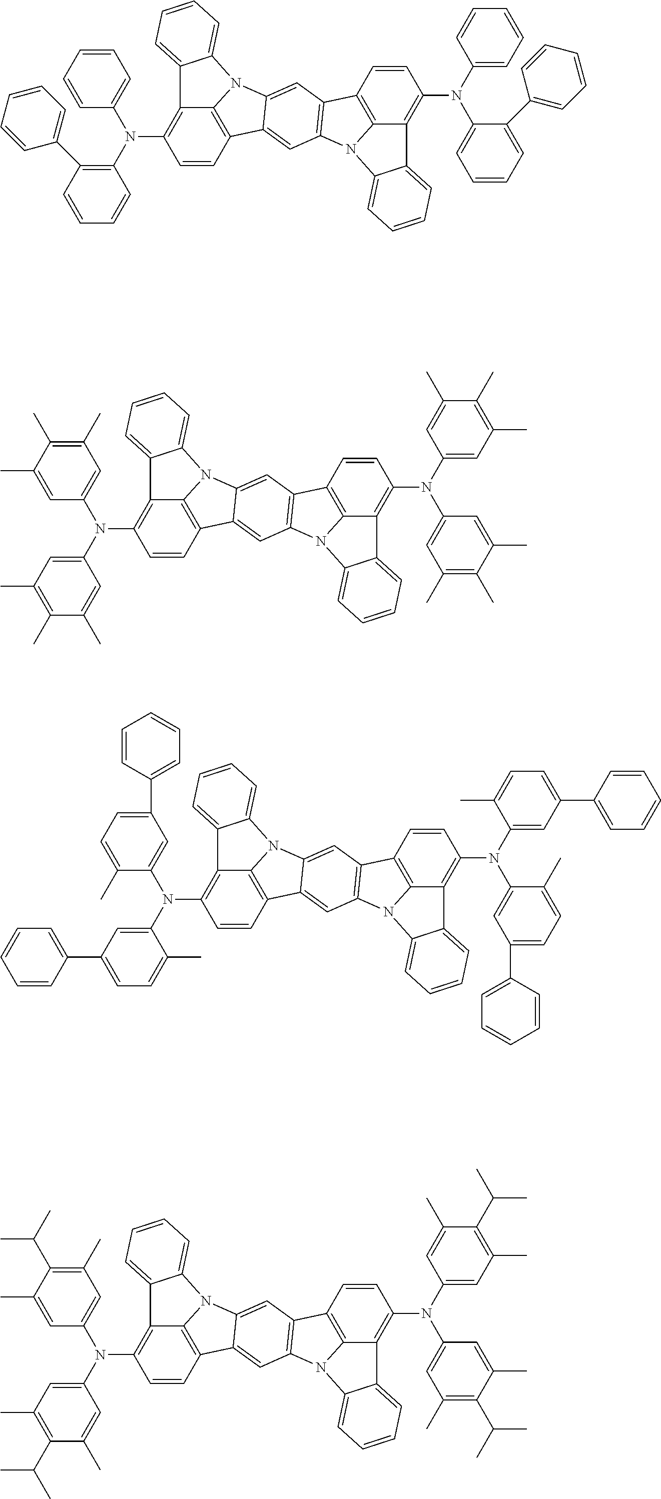

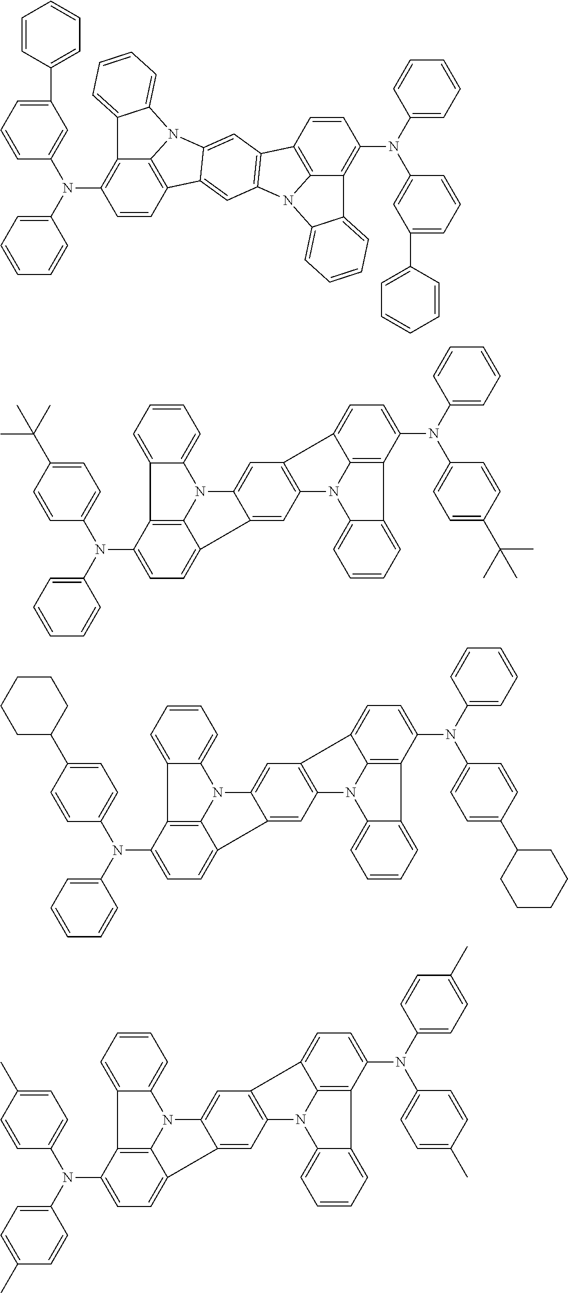

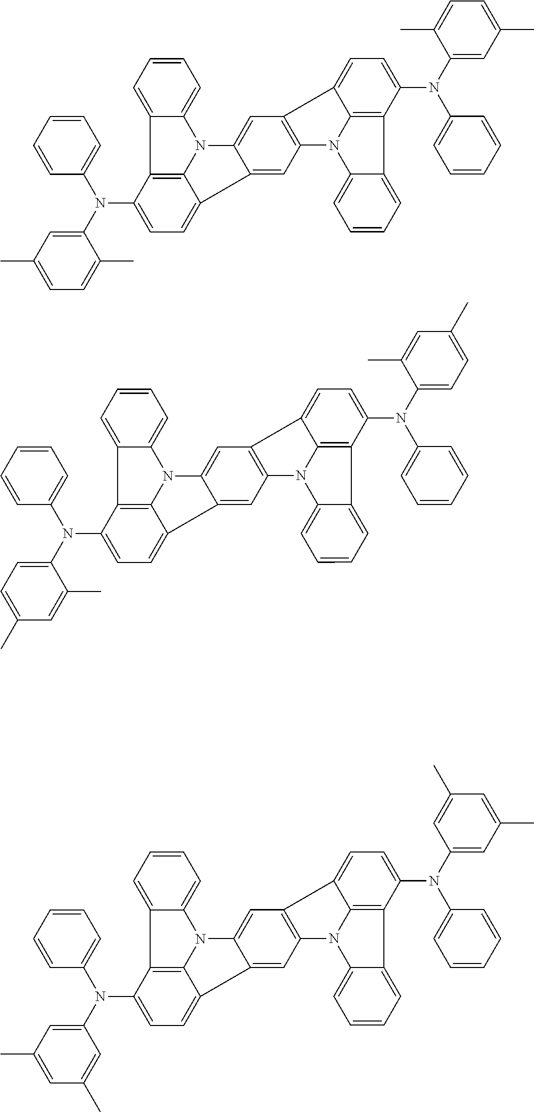

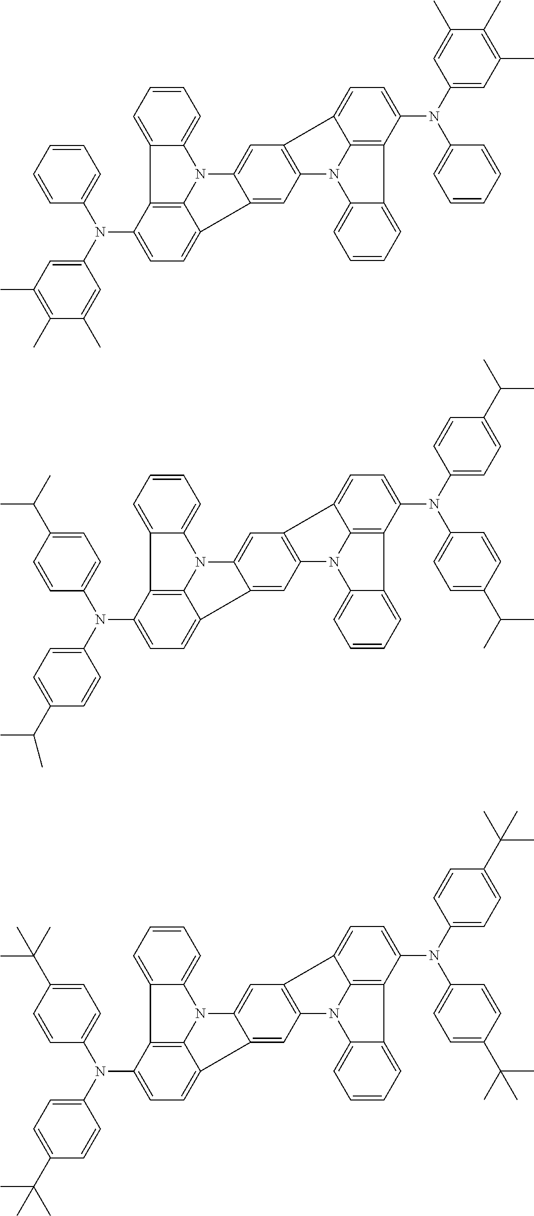

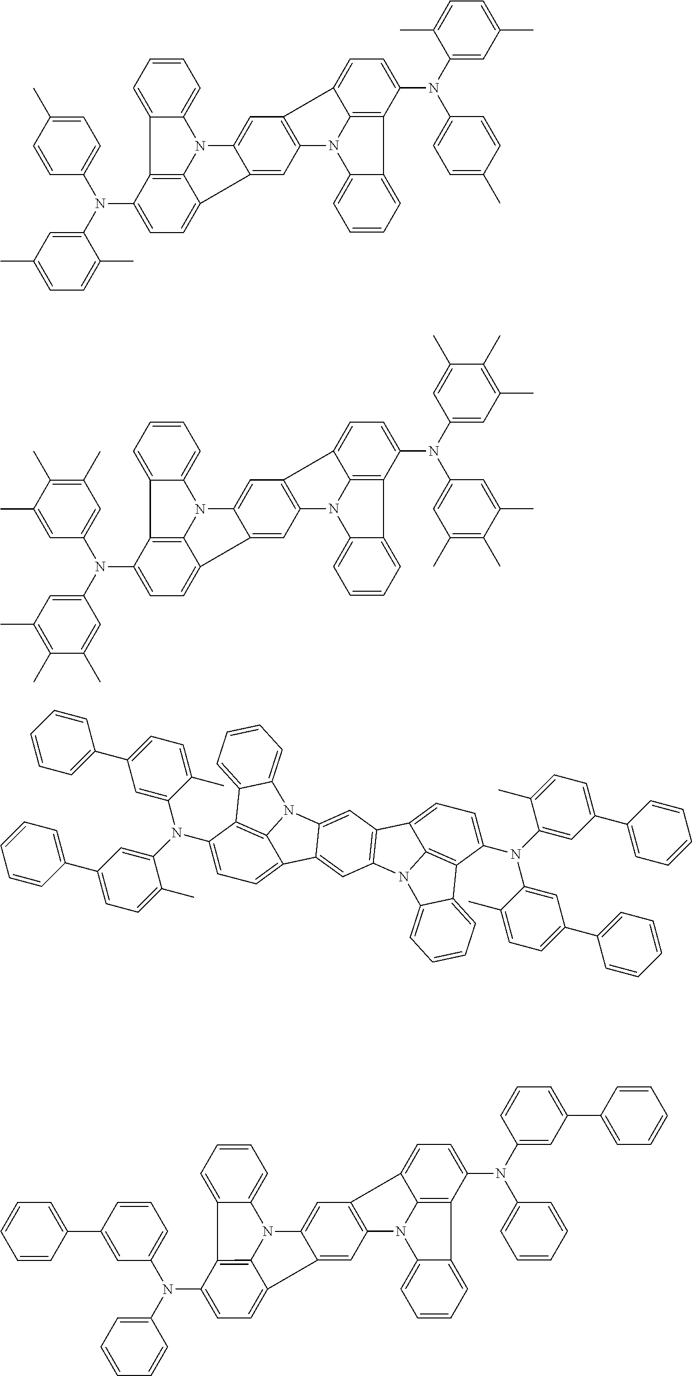

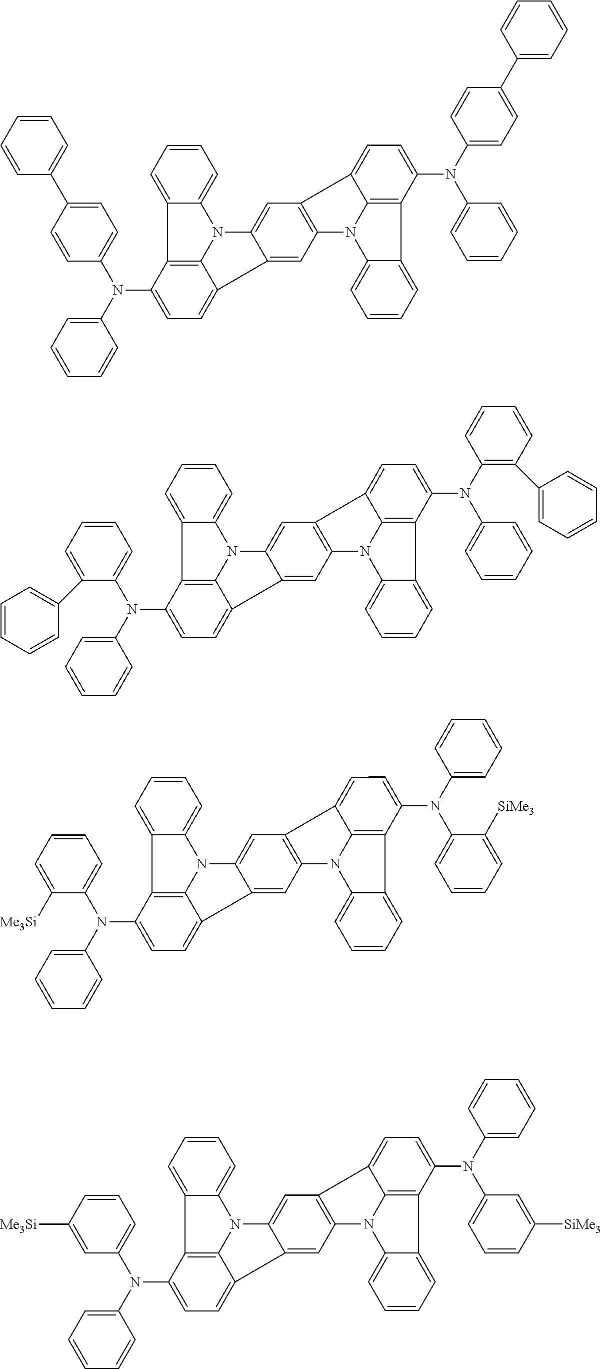

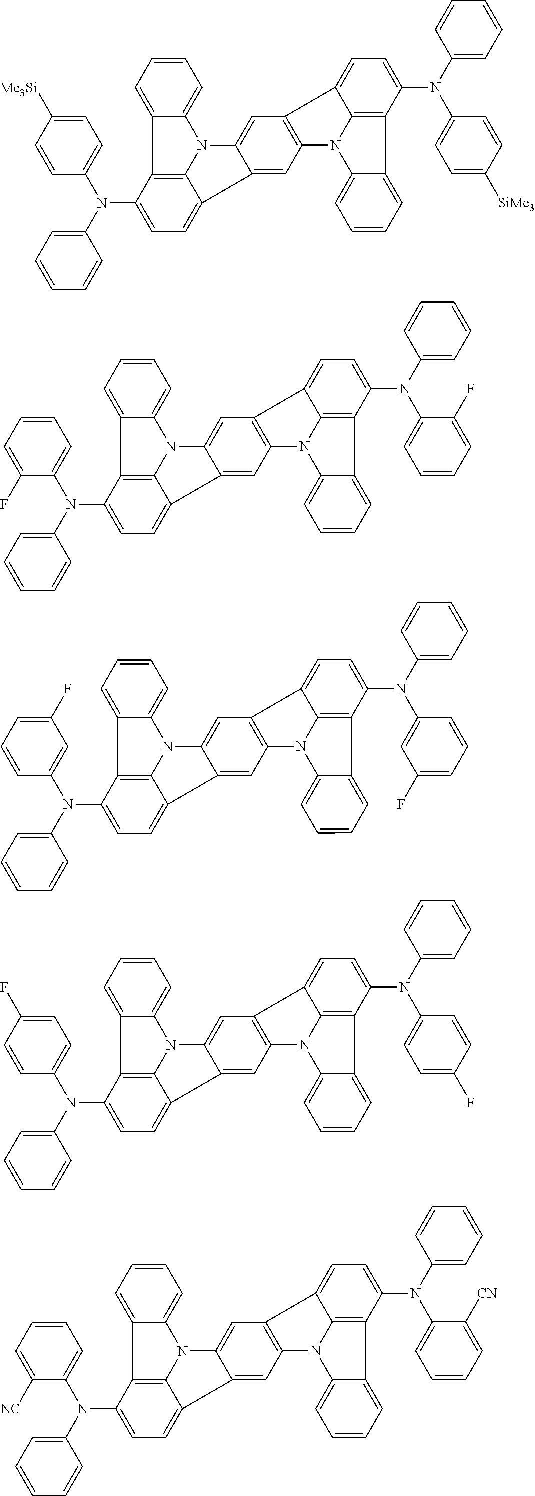

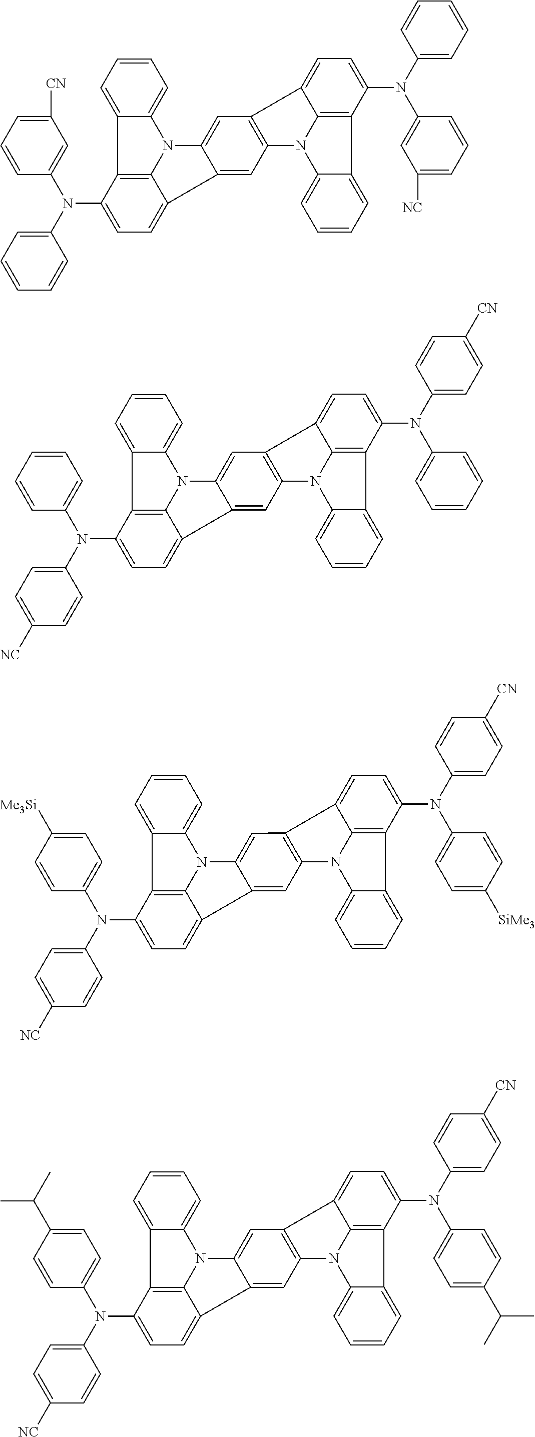

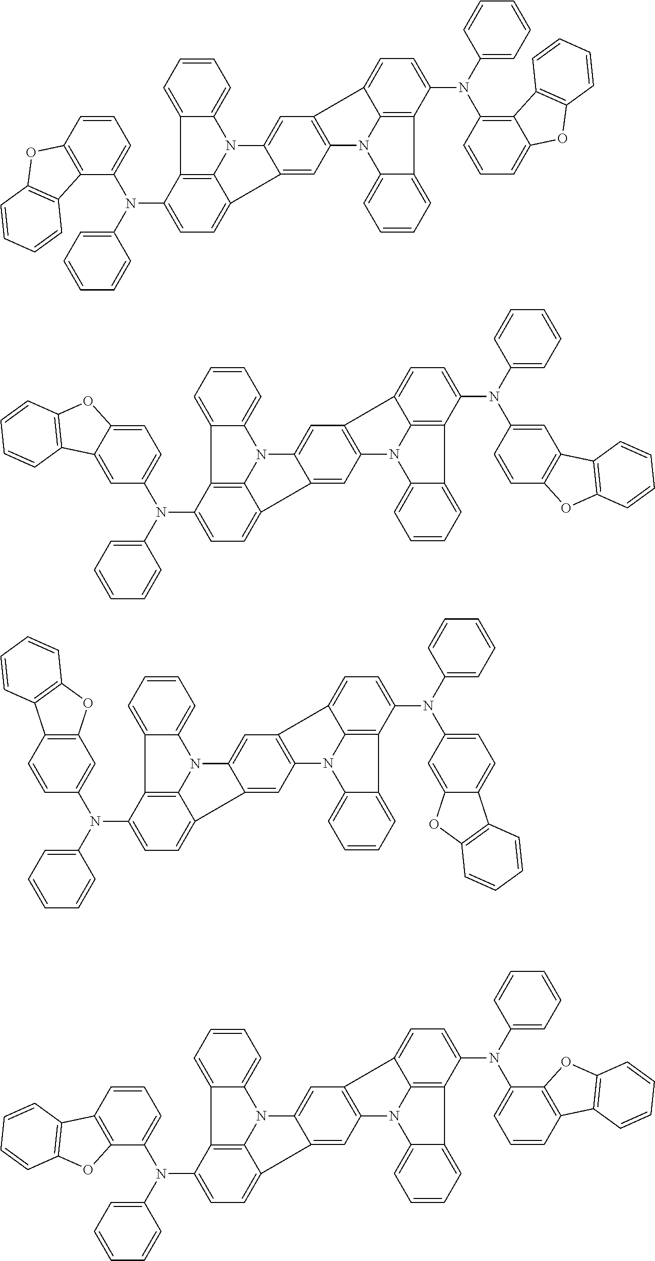

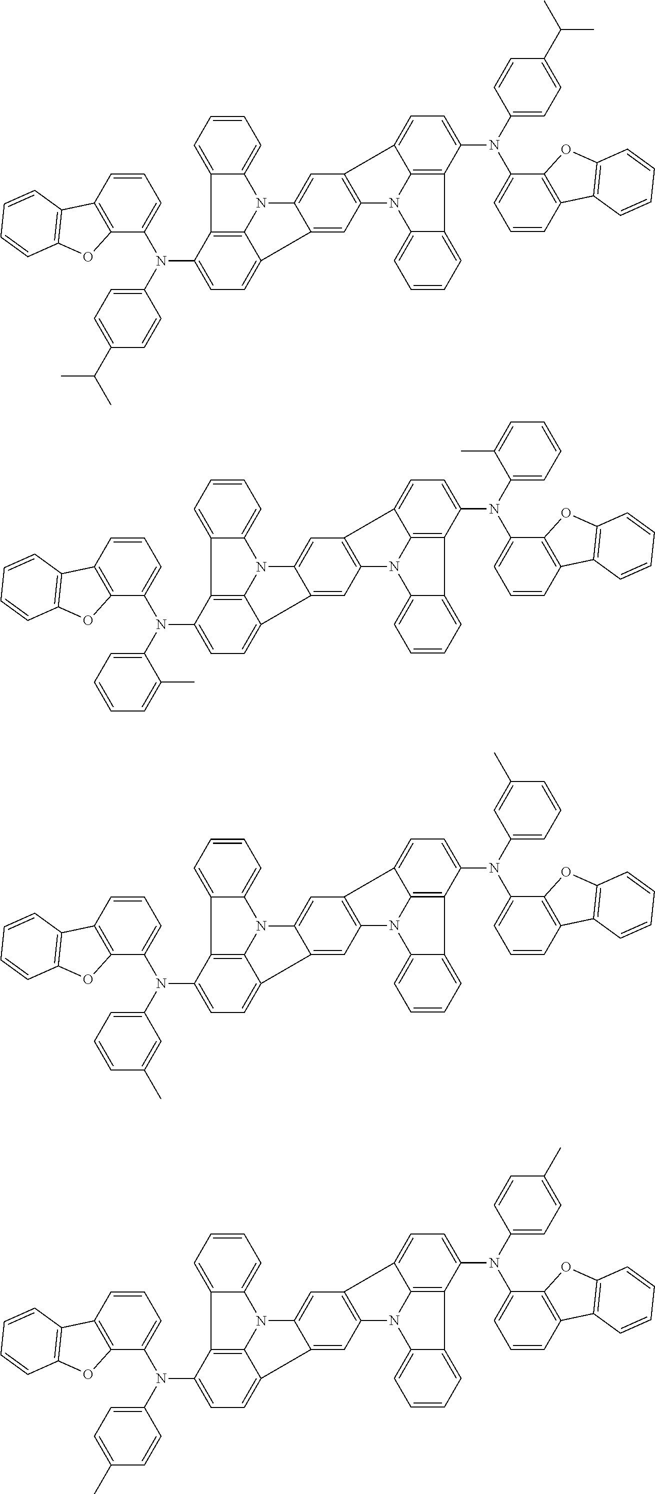

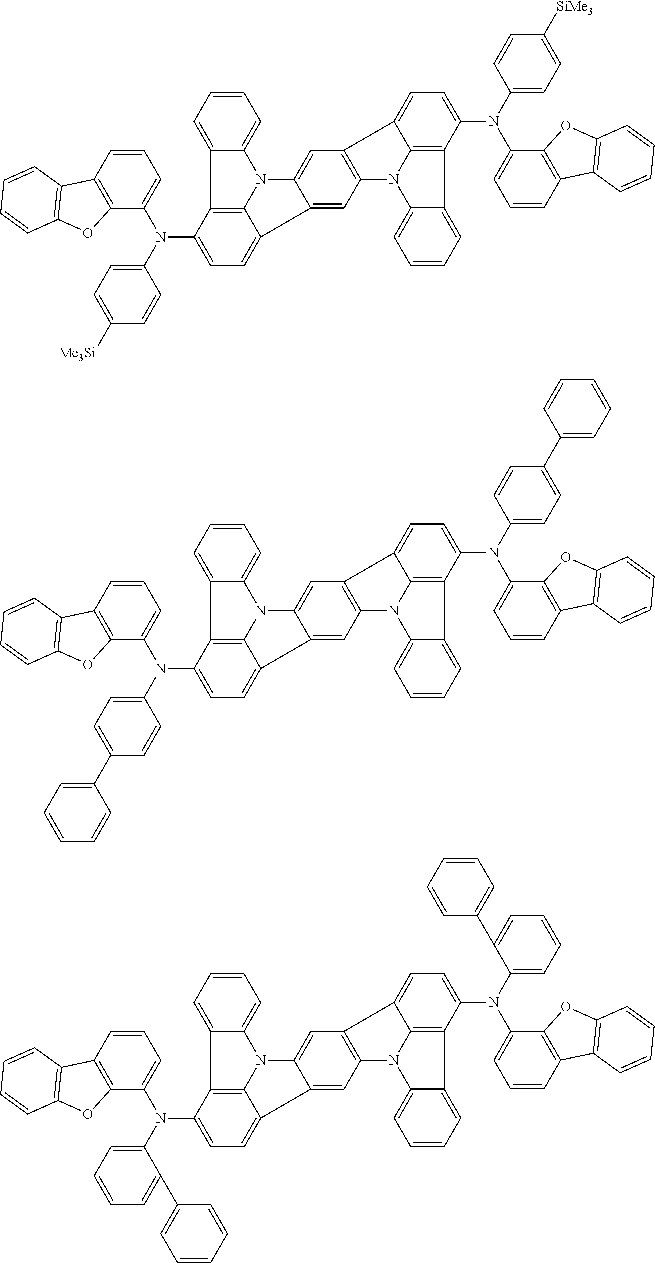

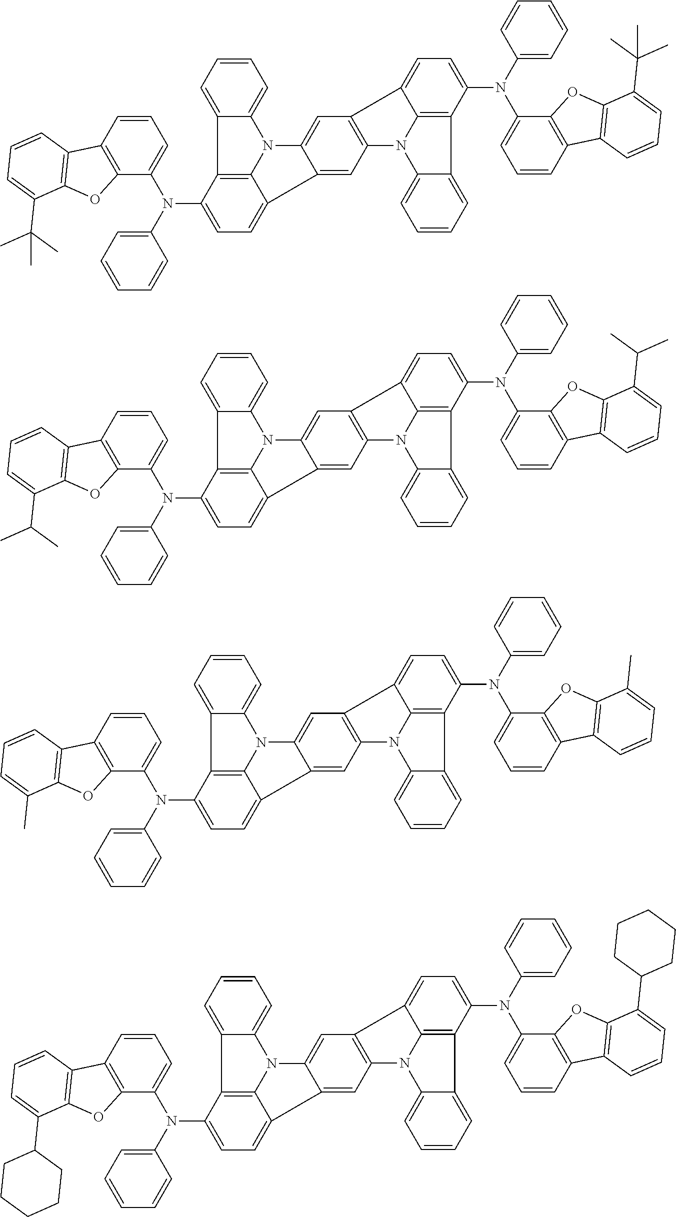

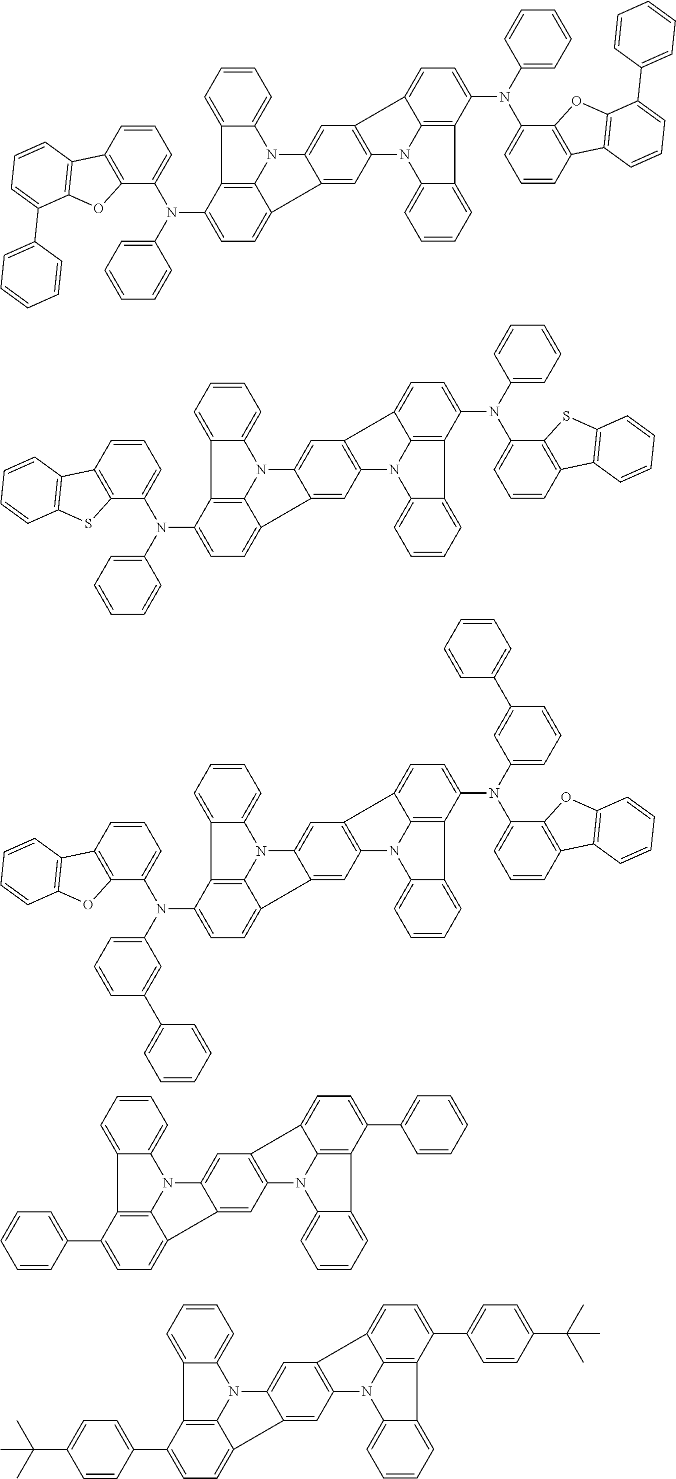

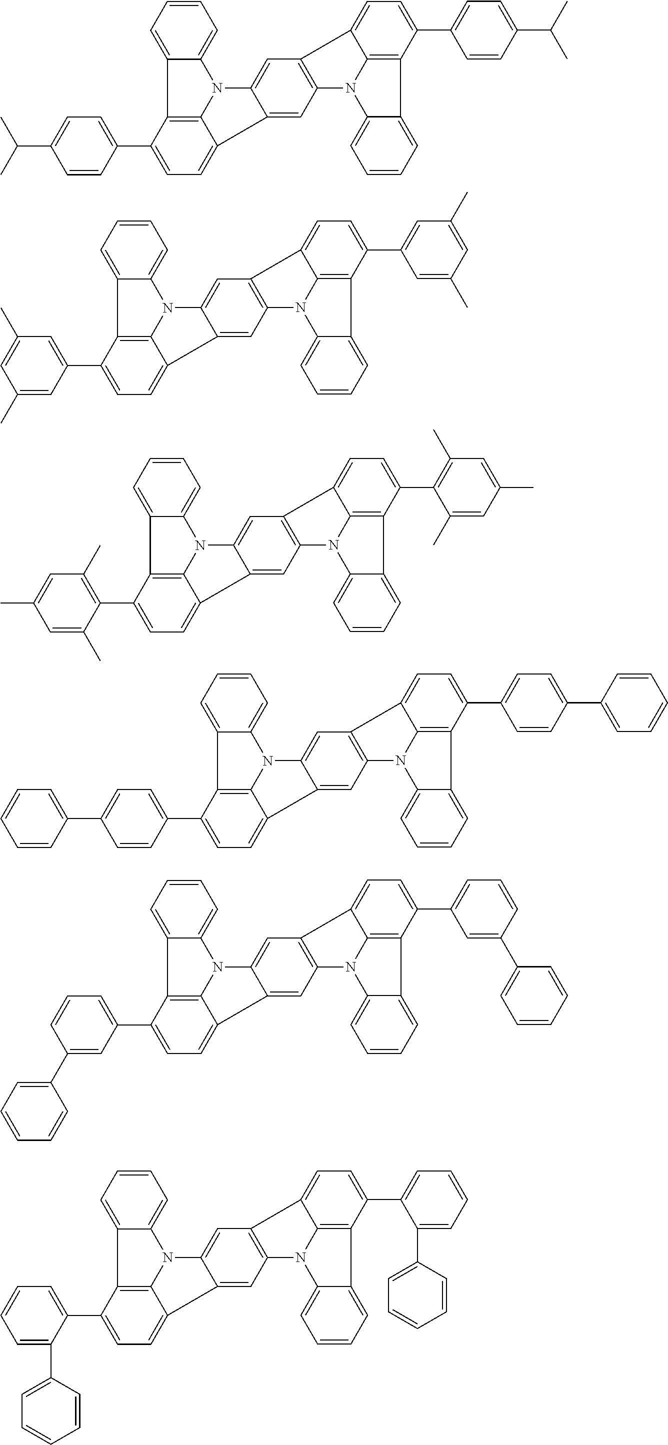

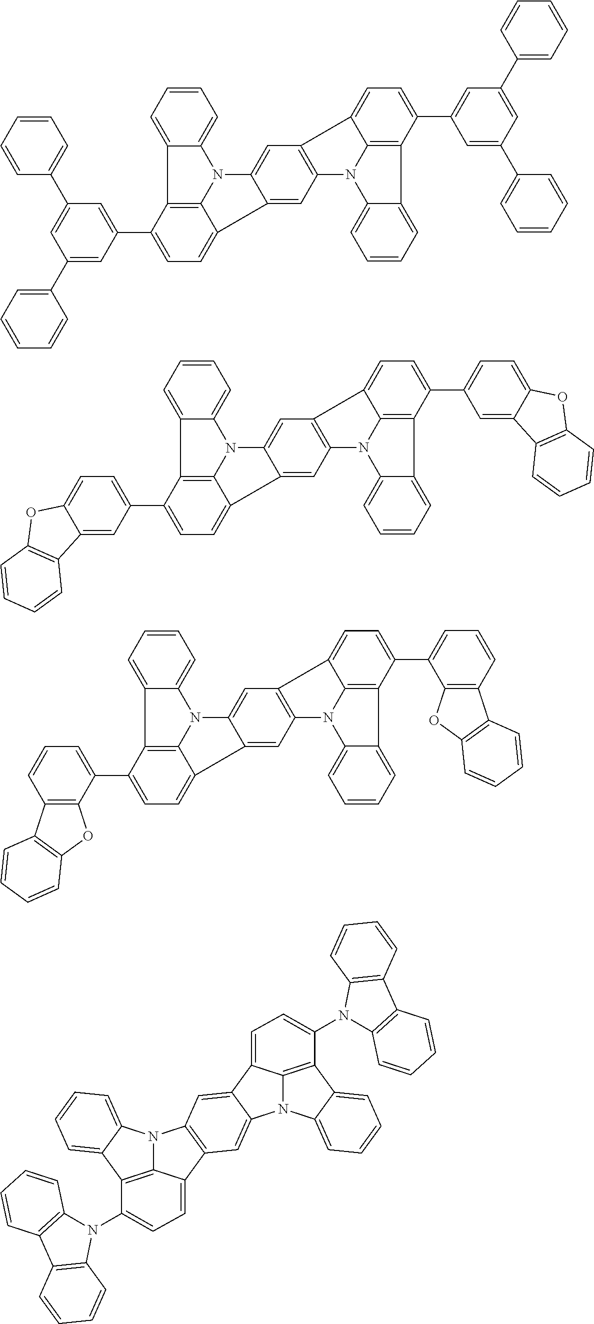

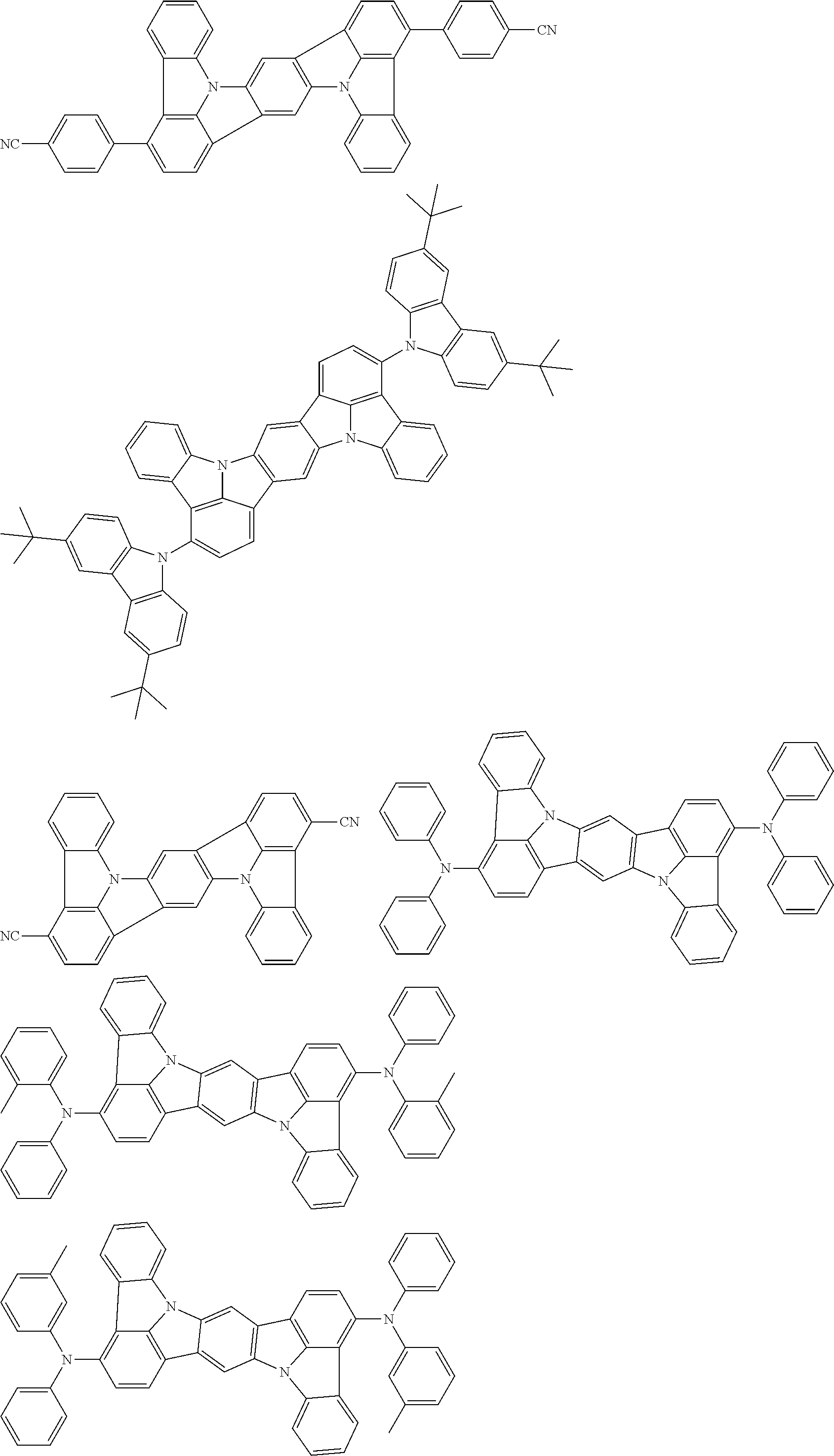

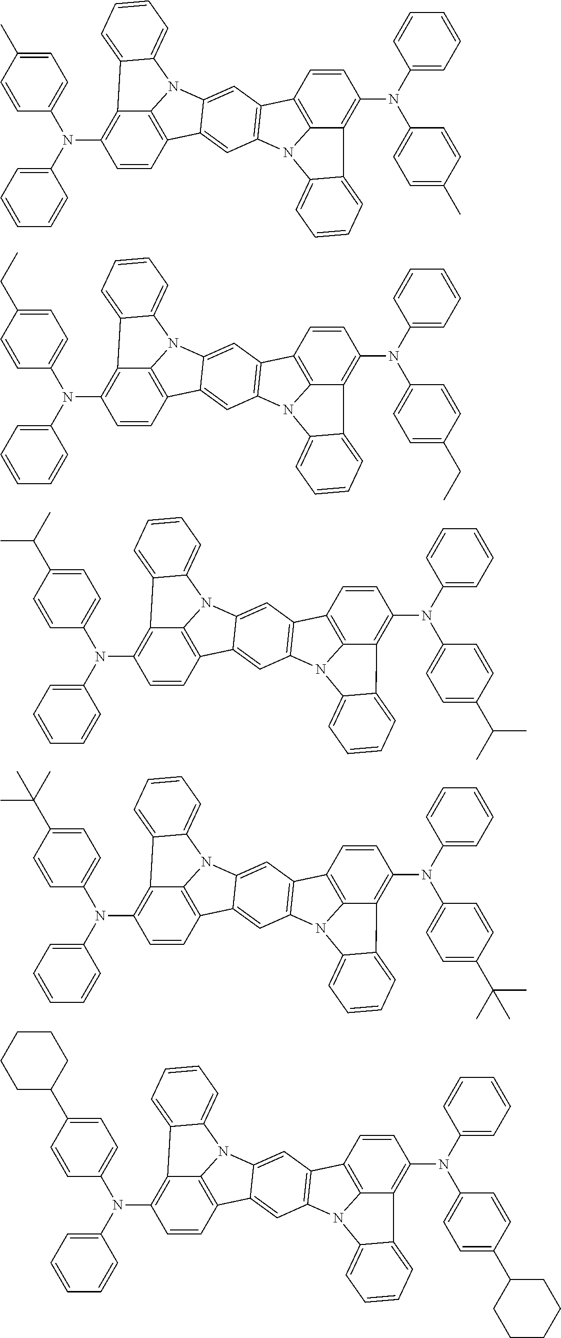

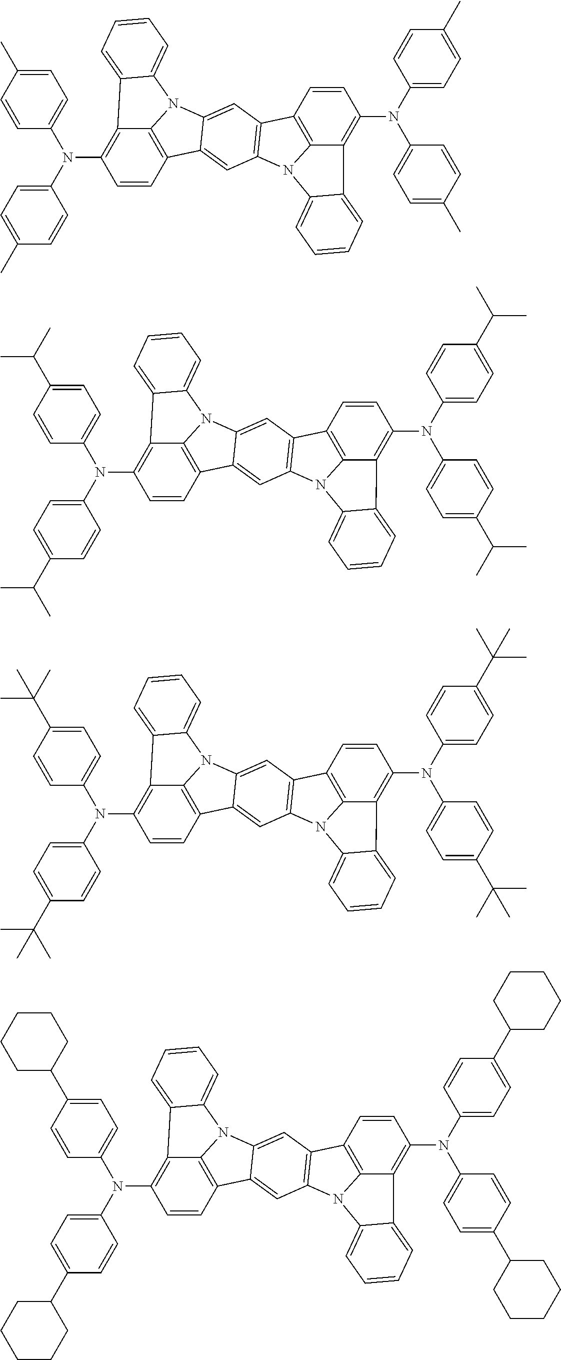

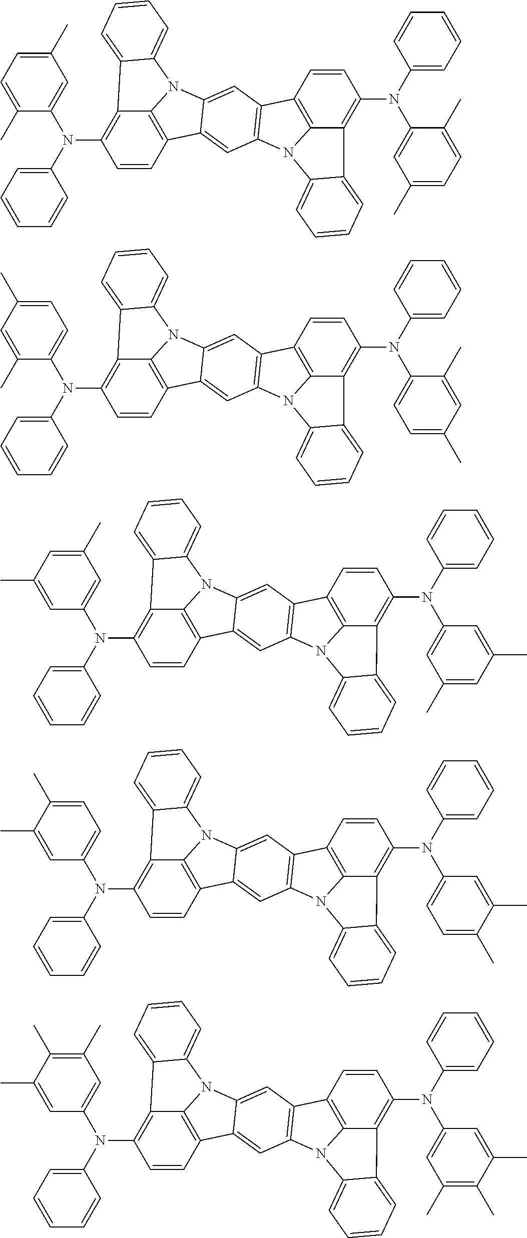

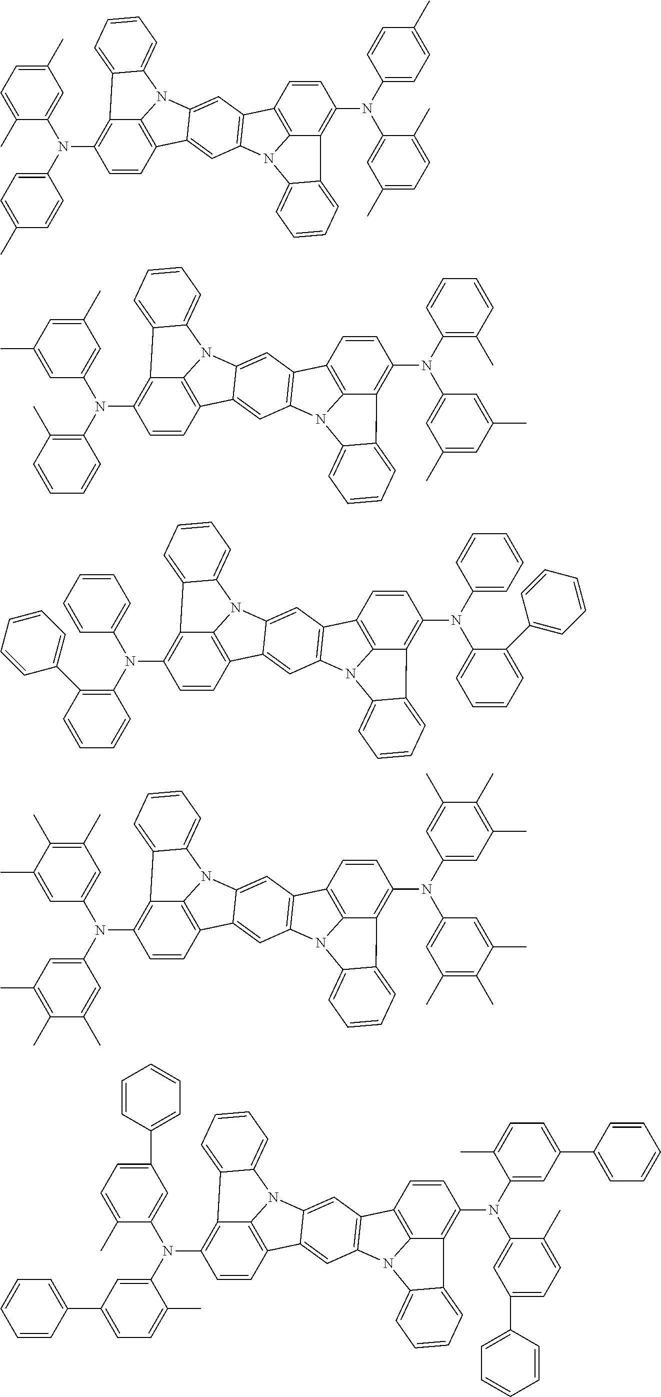

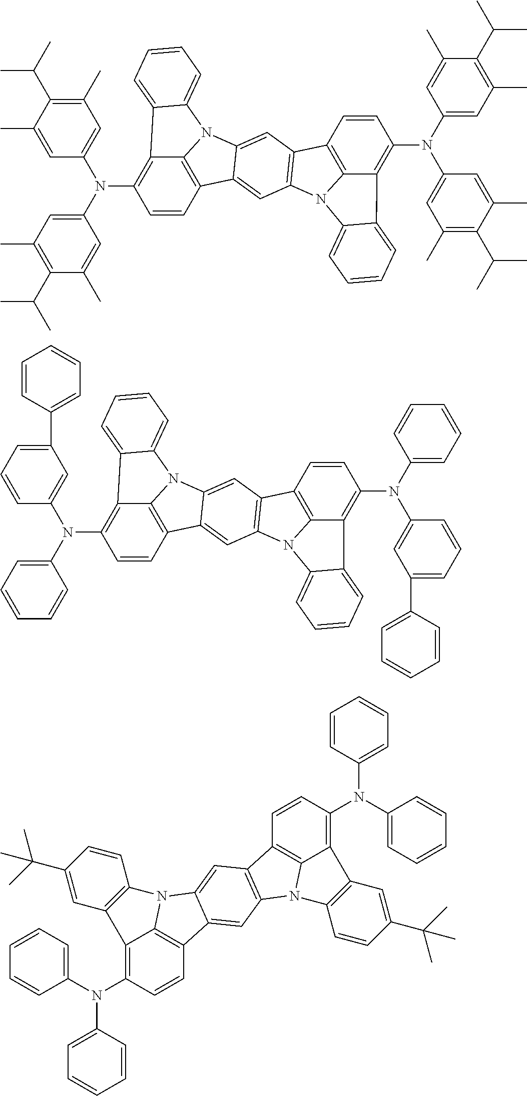

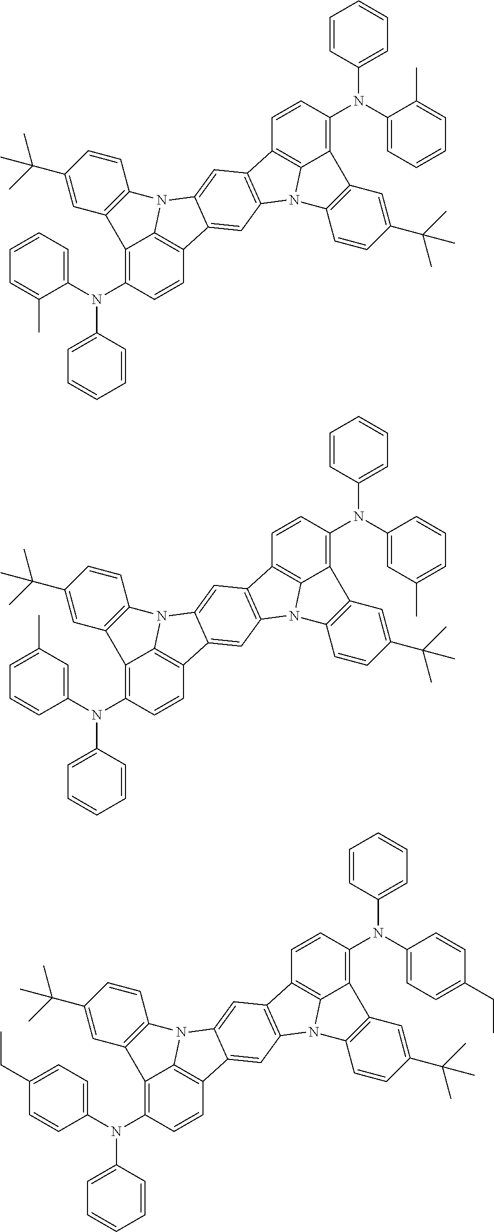

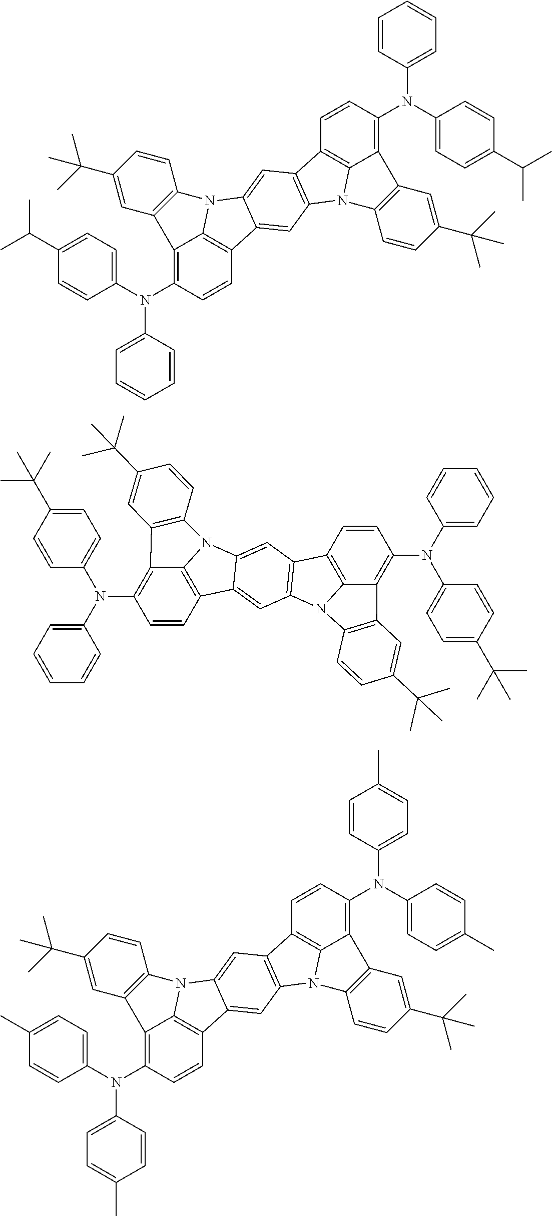

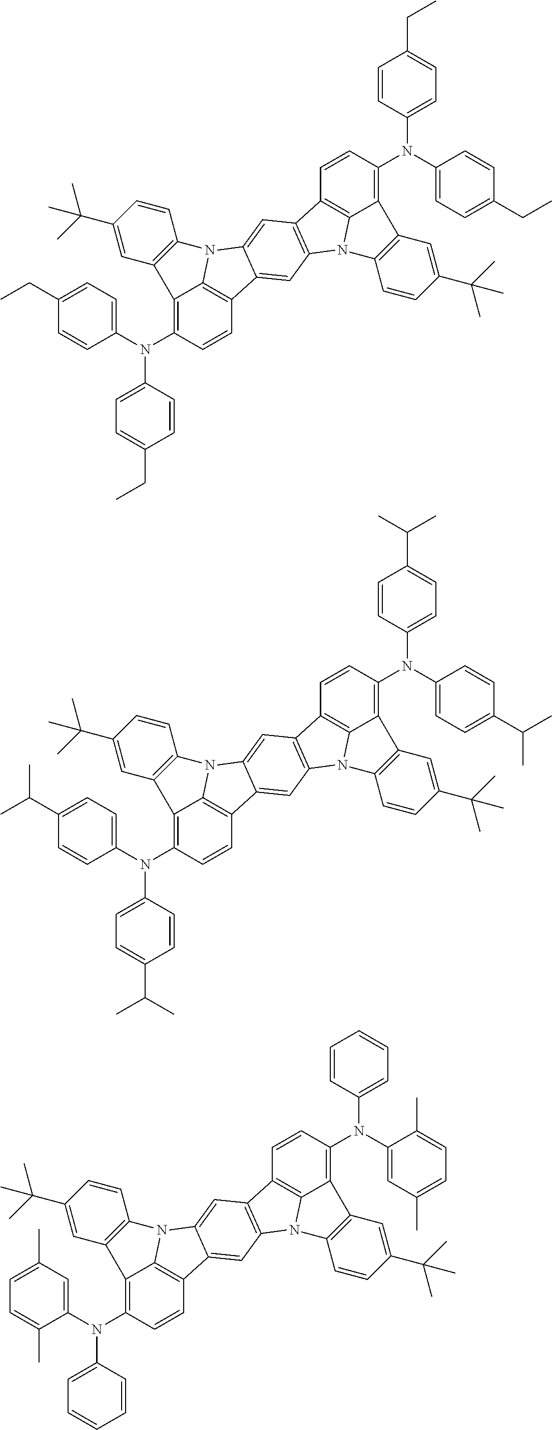

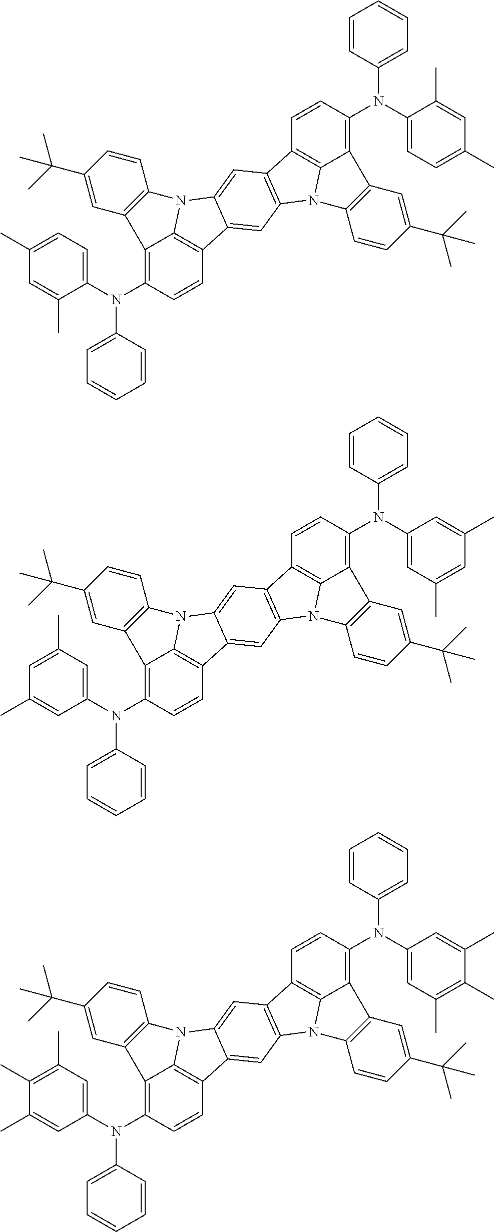

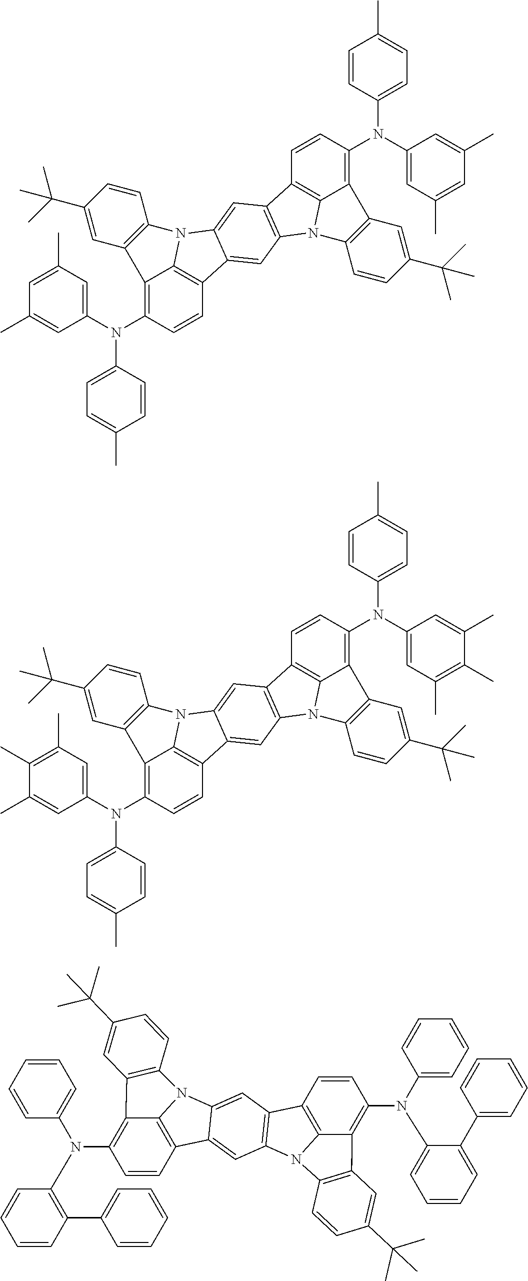

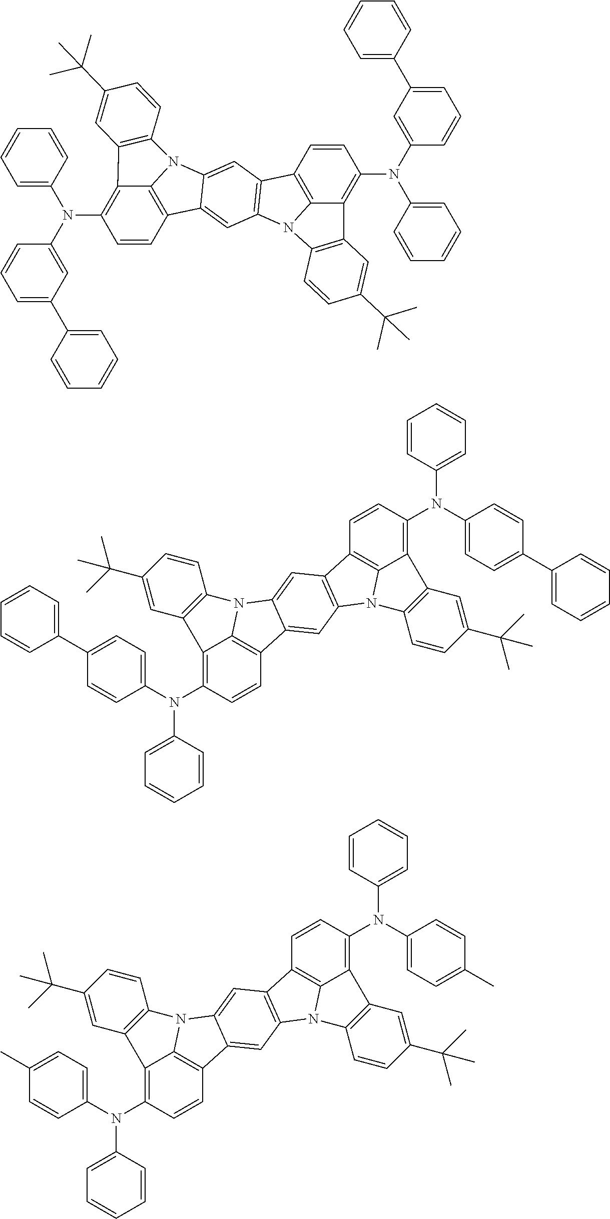

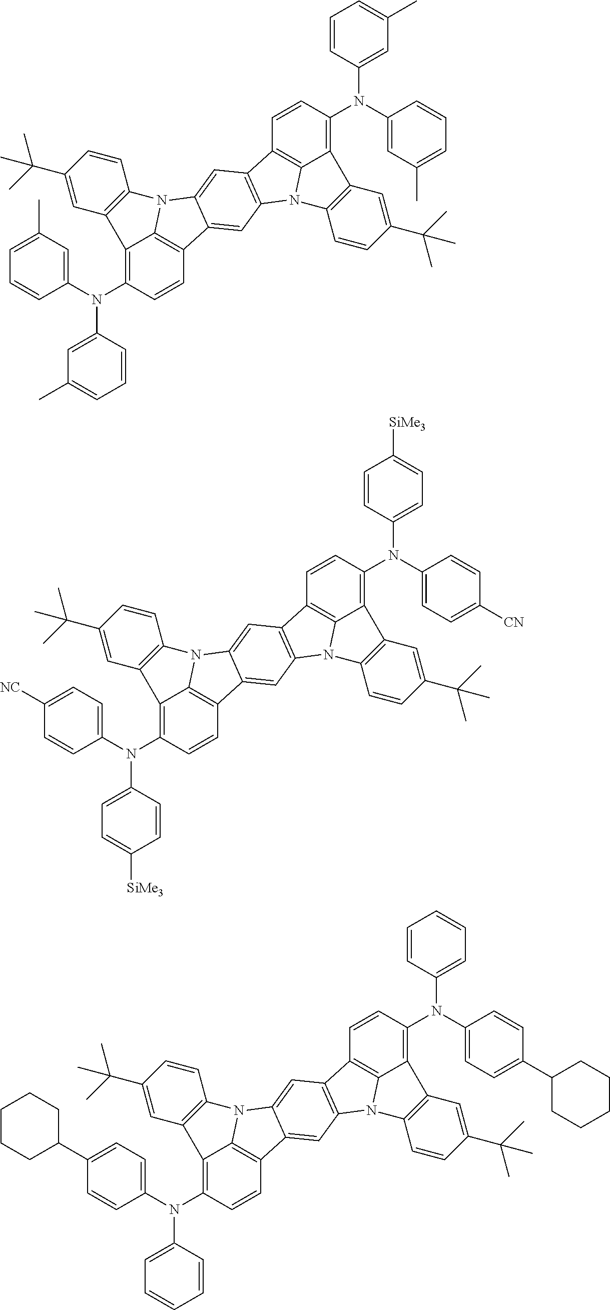

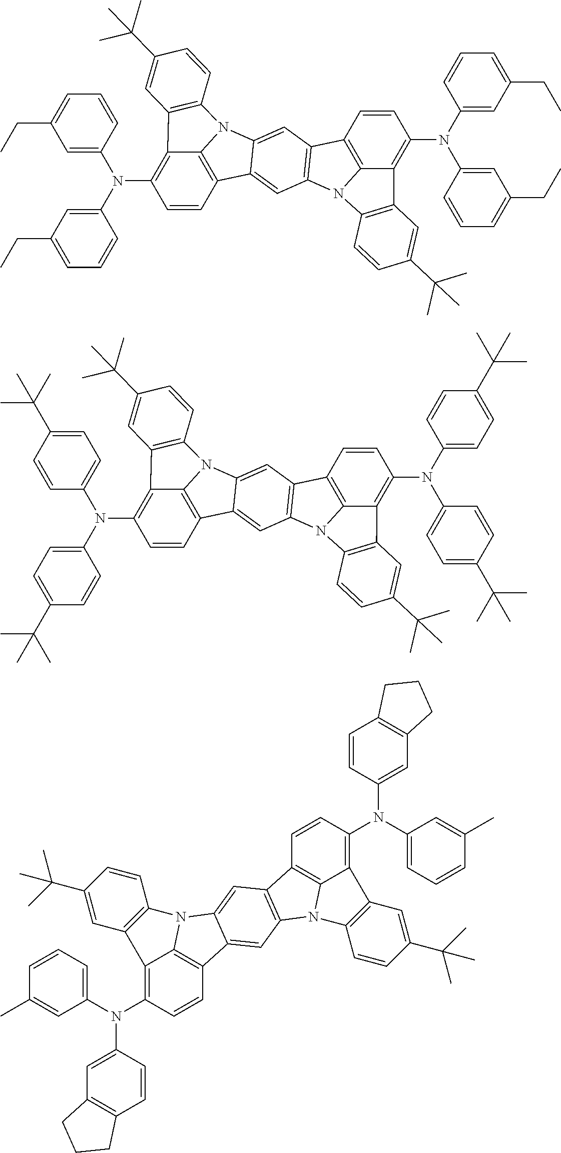

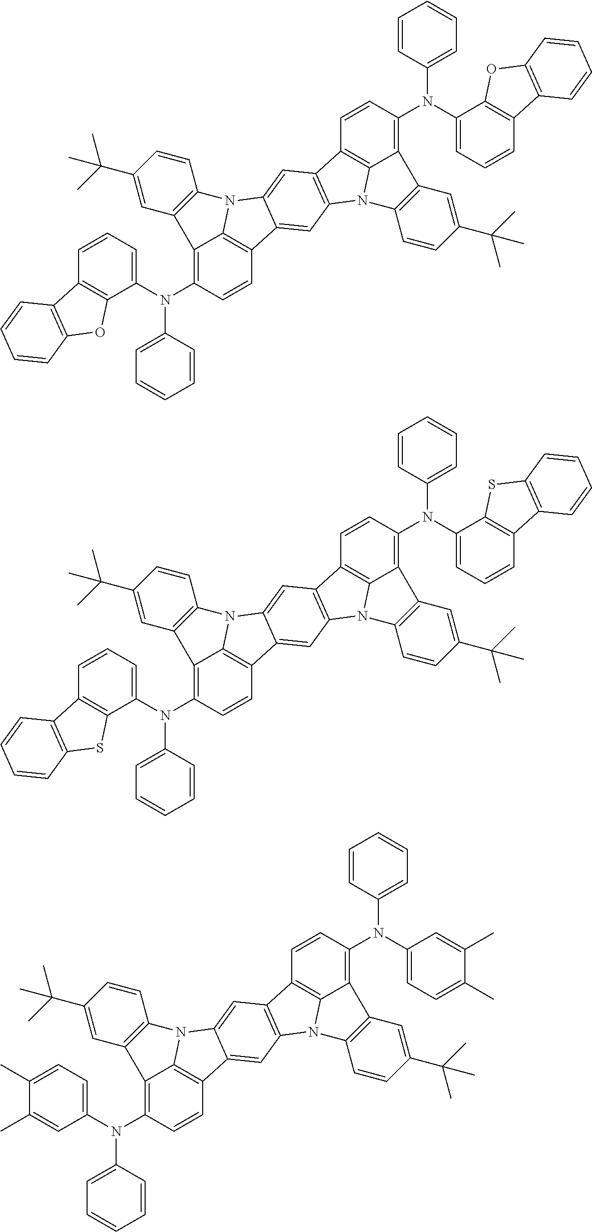

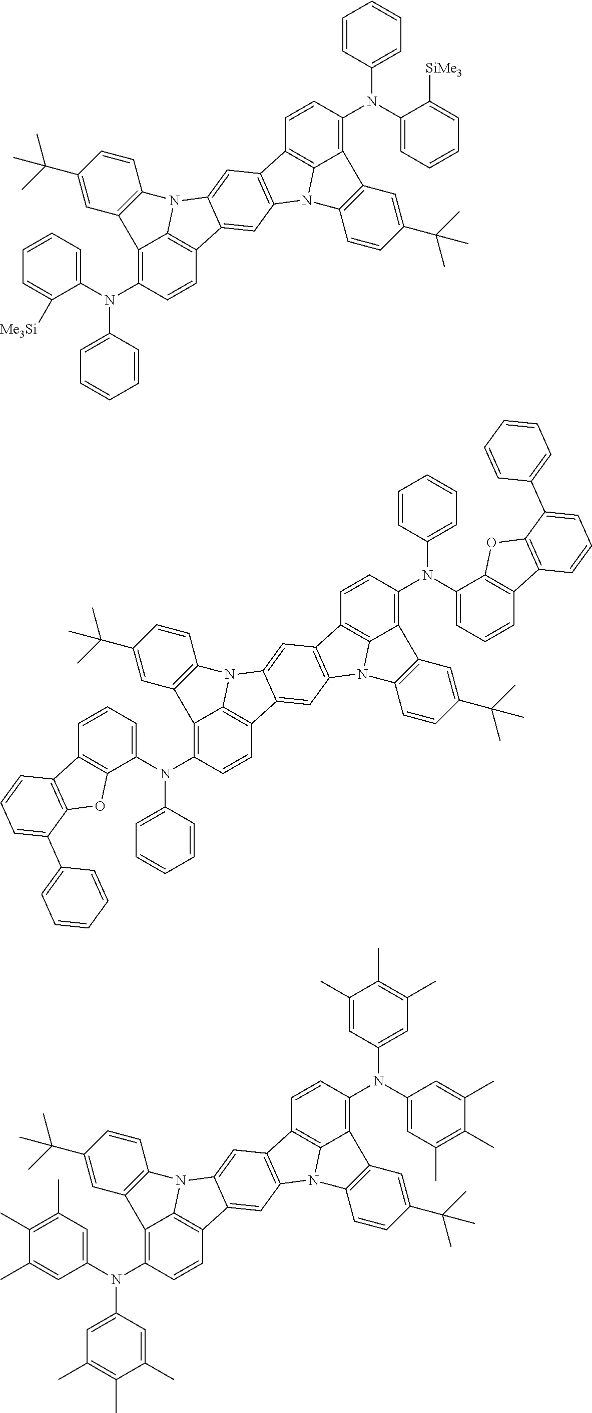

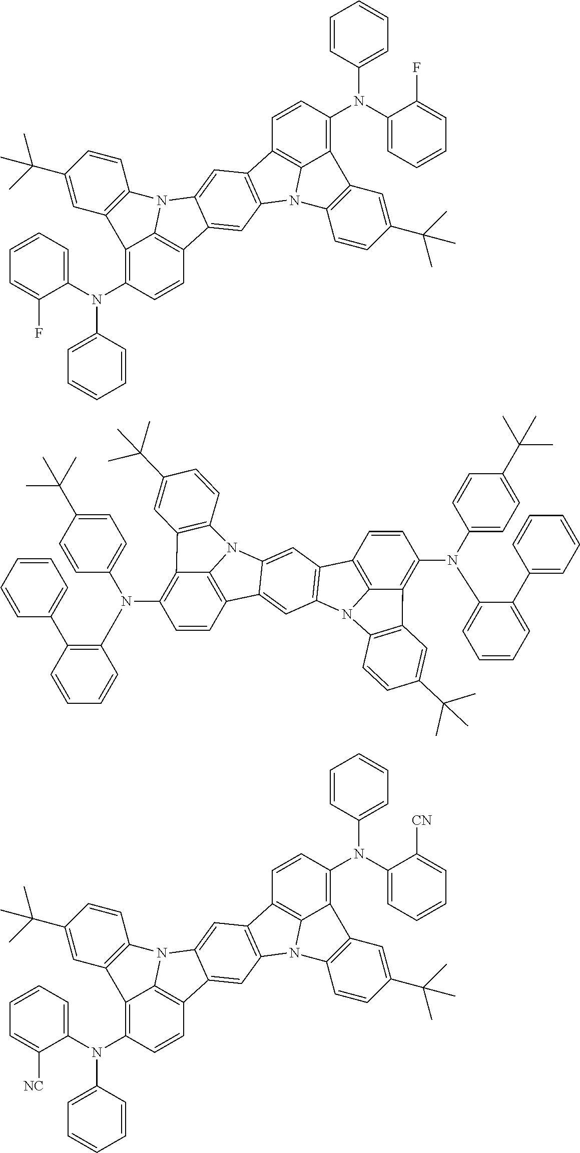

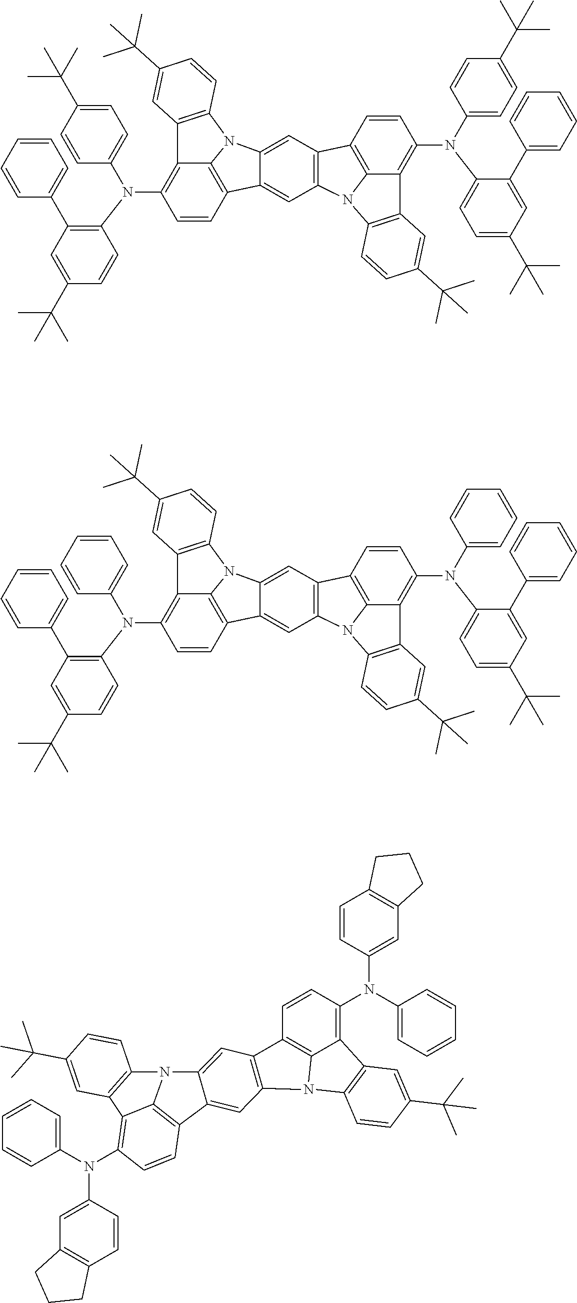

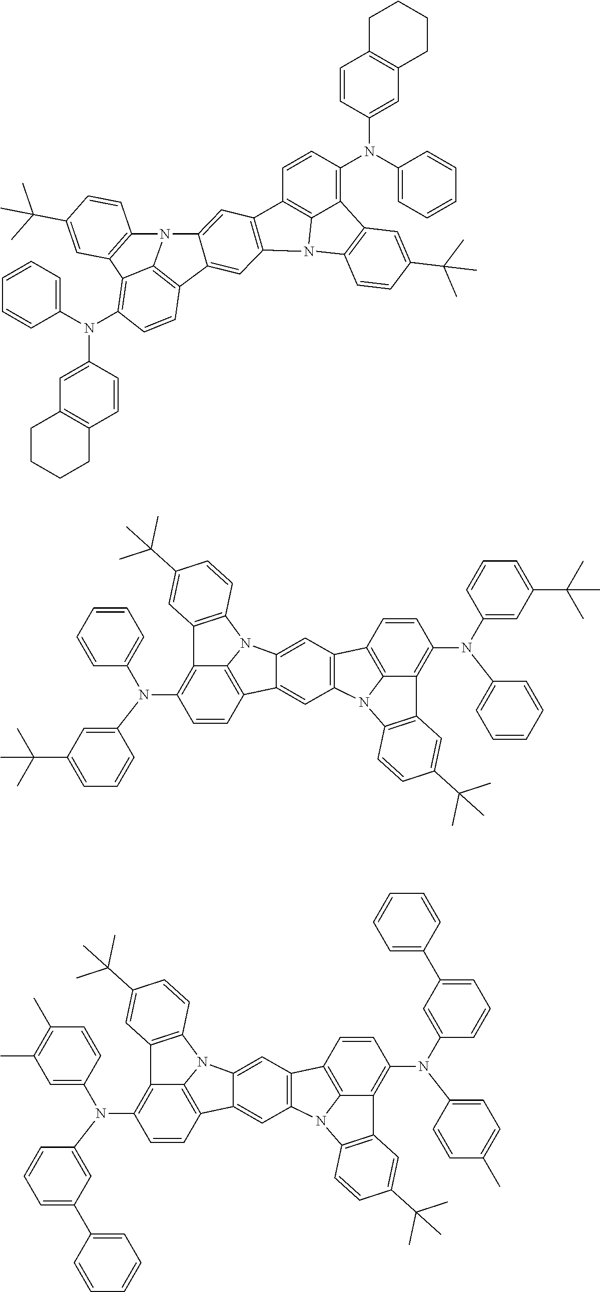

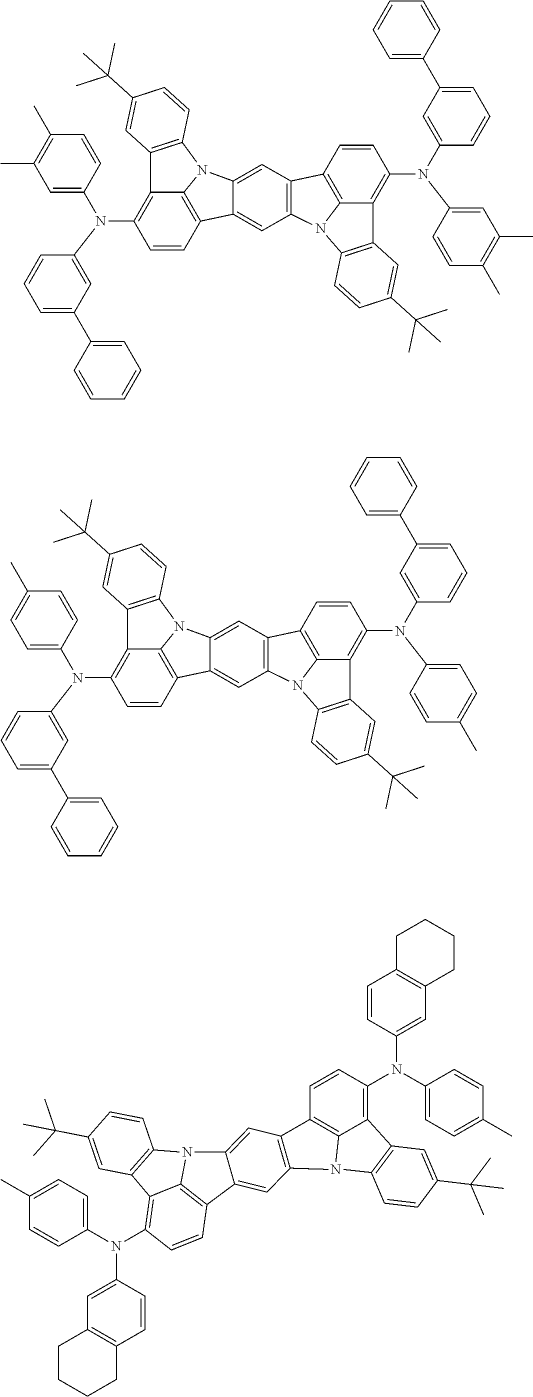

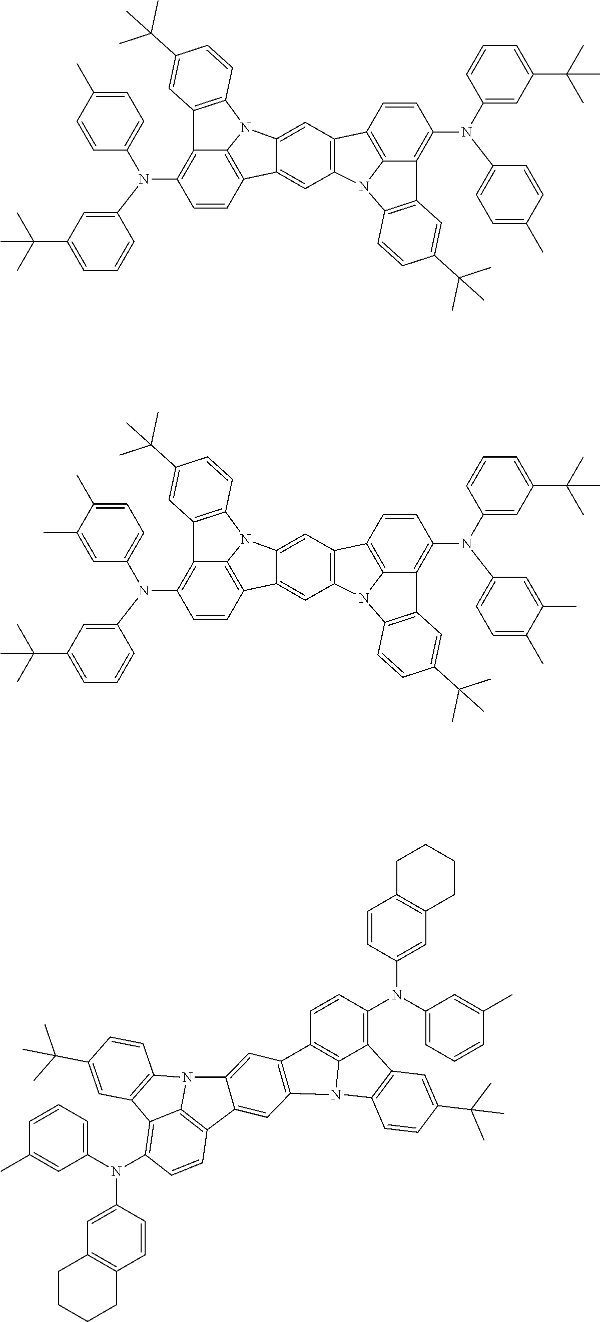

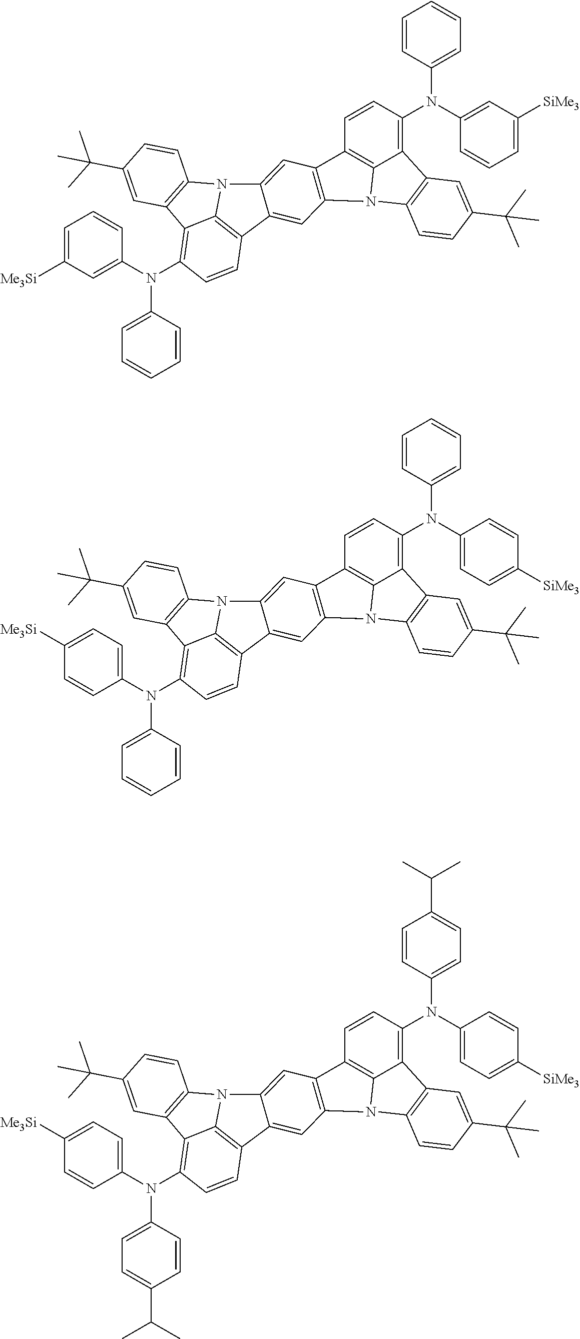

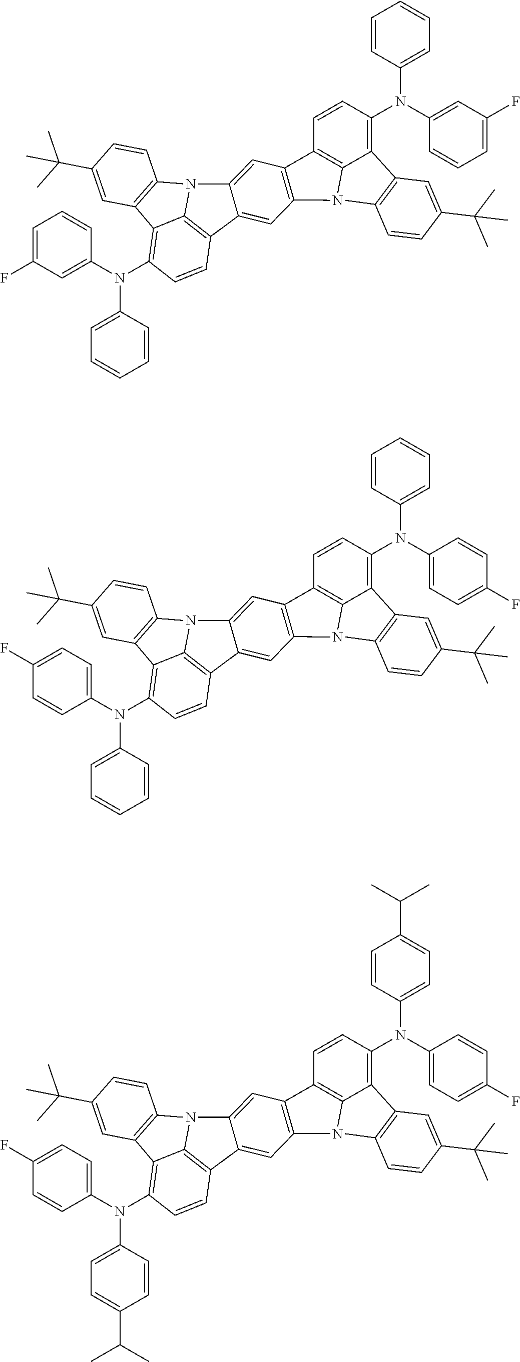

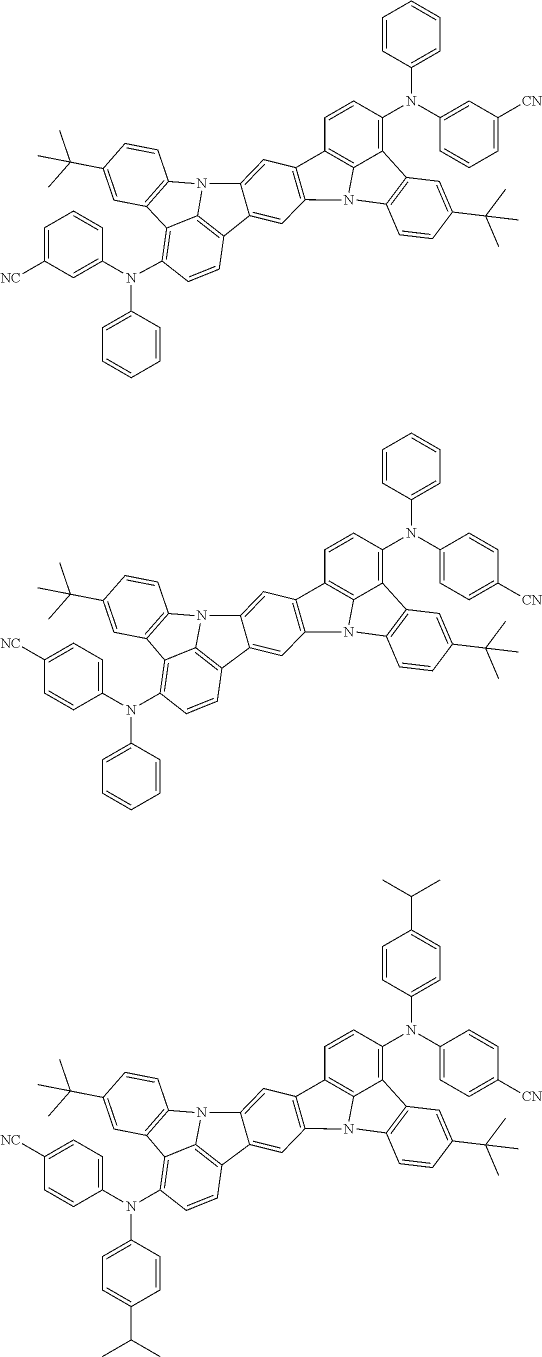

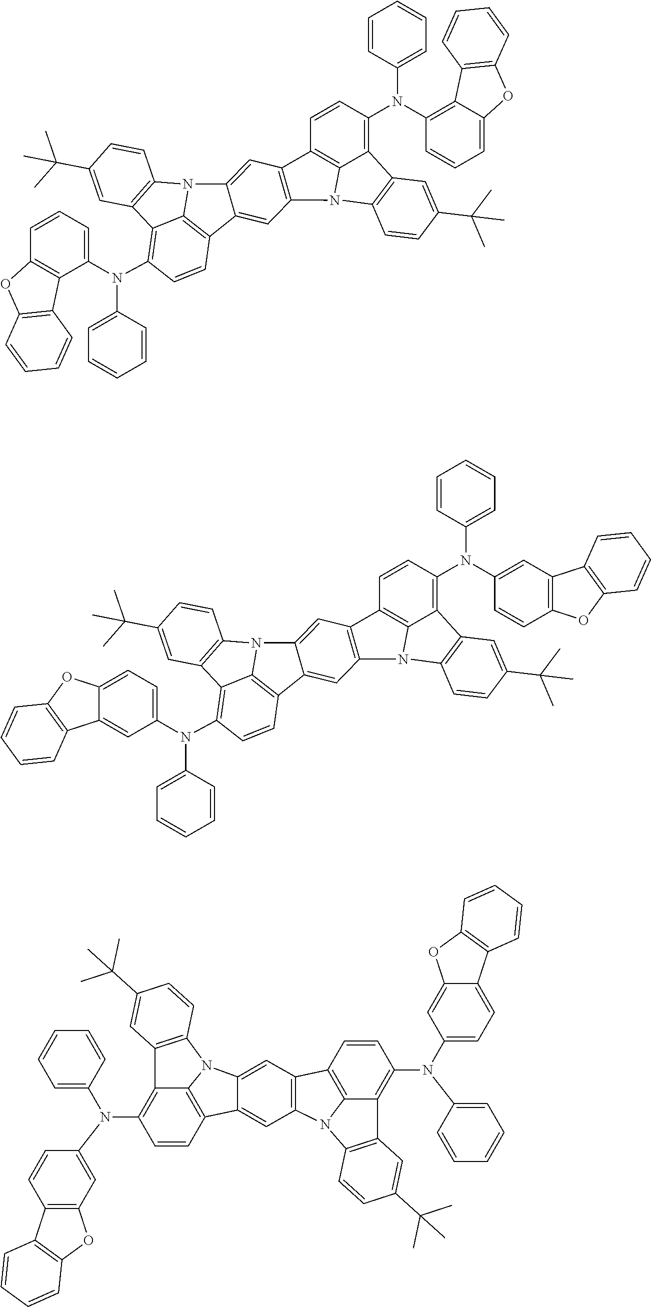

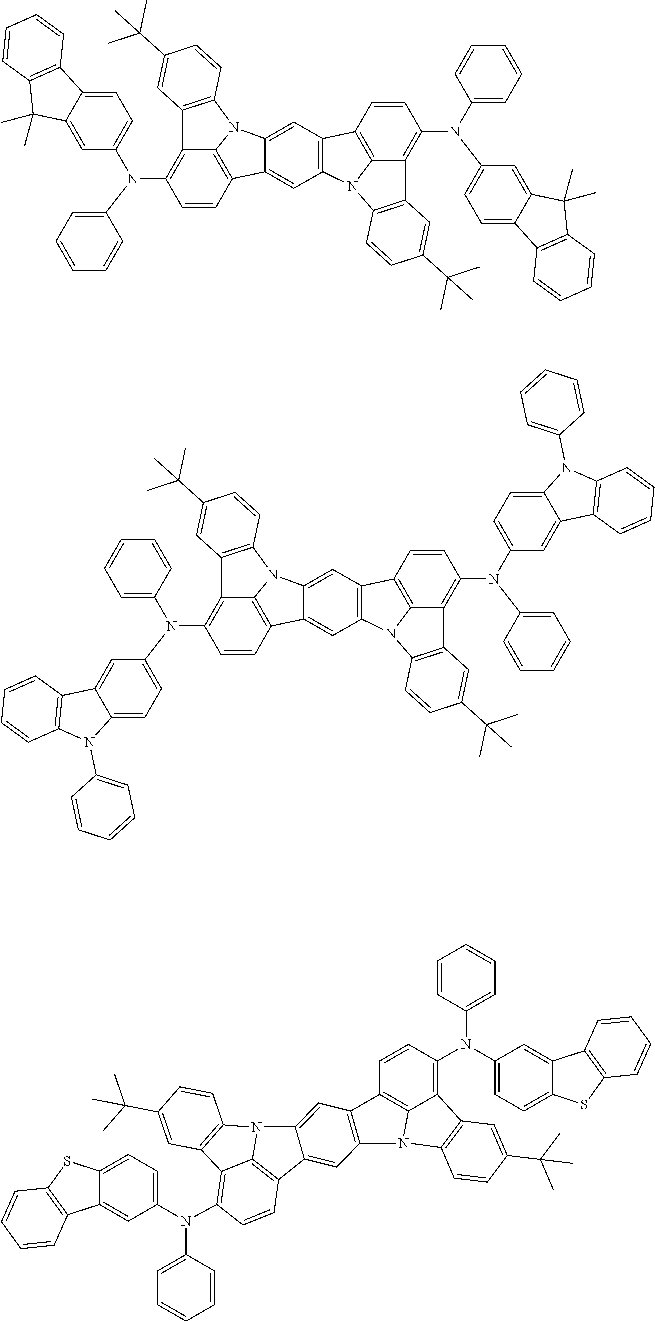

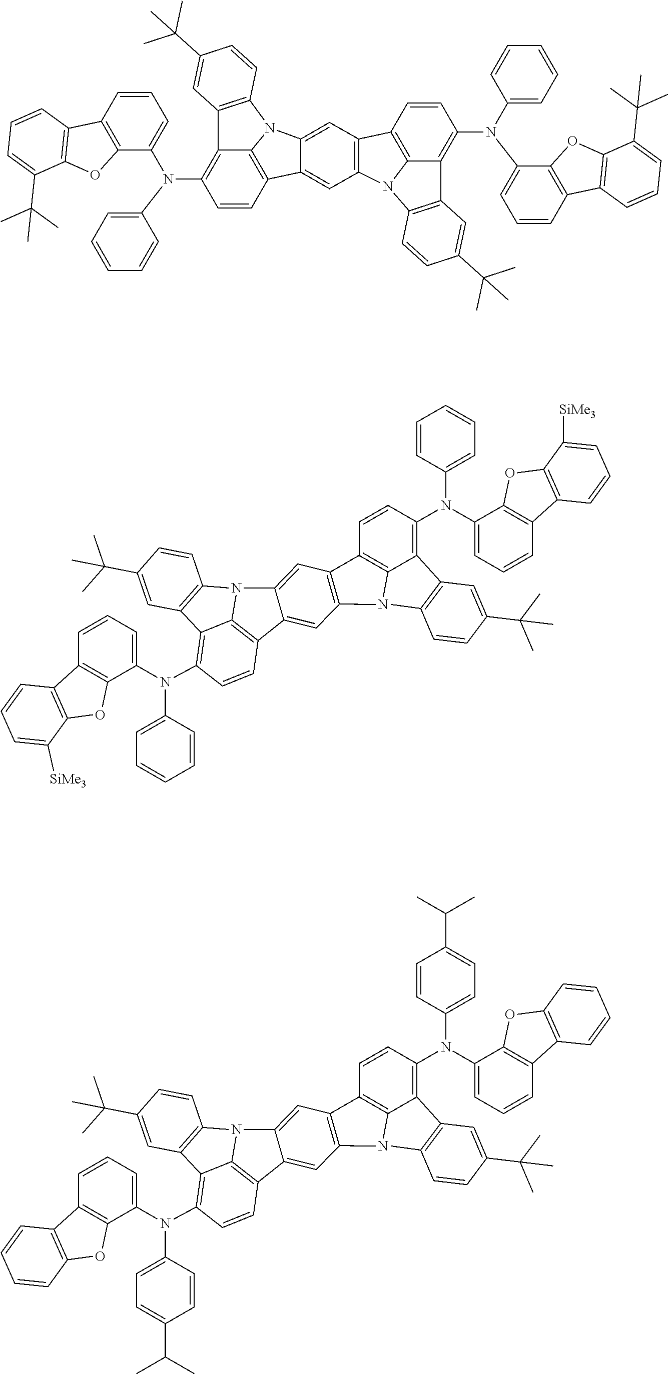

(Compound represented by the general formula (6))

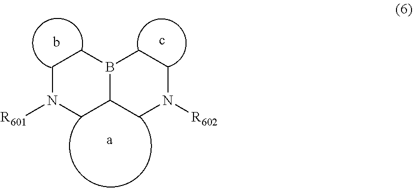

In the general formula (6),
a ring a, a ring b and a ring c are independently
a substituted or unsubstituted aromatic hydrocarbon ring having 6 to 50 ring carbon atoms, or
a substituted or unsubstituted heterocycle having 5 to 50 ring atoms;
R₆₀₁ and R₆₀₂ independently form a substituted or unsubstituted heterocycle by bonding with the ring a, the ring b or the ring c, or do not form the substituted or unsubstituted heterocycle;
R₆₀₁ and R₆₀₂ which do not form the substituted or unsubstituted heterocycle are independently
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms.

The ring a, the ring b and the ring c are a ring (a substituted or unsubstituted aromatic hydrocarbon ring having 6 to 50 ring carbon atoms, or a substituted or unsubstituted heterocycle having 5 to 50 ring atoms) fused with the fused two-ring structure constituted of a boron atom and two nitrogen atoms in the middle of the general formula (6).

The “aromatic hydrocarbon ring” of the ring a, the ring b and the ring c is the same structure as a compound in which hydrogen atoms are introduced in the above-mentioned “aryl group”.

The “aromatic hydrocarbon ring” of the ring a includes three carbon atoms in the fused two-ring structure in the middle of the general formula (6) as ring atoms.

The “aromatic hydrocarbon ring” of the ring b and the ring c includes two carbon atoms in the fused two-ring structure in the middle of the general formula (6) as ring atoms.

Specific examples of the “substituted or unsubstituted aromatic hydrocarbon ring having 6 to 50 ring carbon atoms” include a compound in which hydrogen atoms are introduced in the “aryl group” described in the specific example group G1, and the like.

The “heterocycle” of the ring a, the ring b and the ring c is the same structure as a compound in which hydrogen atoms are introduced in the above-mentioned “heterocyclic group”.

The “heterocycle” of the ring a includes three carbon atoms in the fused two-ring structure in the middle of the general formula (6) as ring atoms. The “heterocycle” of the ring b and the ring c includes two carbon atoms in the fused two-ring structure in the middle of the general formula (6) as ring atoms. Specific examples of the “substituted or unsubstituted heterocycle having 5 to 50 ring atoms” include a compound in which hydrogen atoms are introduced in the “heterocyclic group” described in the specific example group G2, and the like.

R₆₀₁ and R₆₀₂ independently may form a substituted or unsubstituted heterocycle by bonding with the ring a, the ring b or the ring c. The heterocycle in this case includes a nitrogen atom in the fused two-ring structure in the middle of the general formula (6). The heterocycle in this case may include a heteroatom other than the nitrogen atom. The expression “R₆₀₁ and R₆₀₂ bond with the ring a, the ring b or the ring c” means that specifically, an atom constituting the ring a, the ring b or the ring c and an atom constituting R₆₀₁ and R₆₀₂ are bonded. For example, R₆₀₁ may be bonded with the ring a to form a two-ring-fused (or three or more-ring-fused) nitrogen-containing heterocycle in which a ring containing R₆₀₁ and the ring a are fused. Specific examples of the nitrogen-containing heterocycle include a compound corresponding to a heterocyclic group containing a nitrogen atom and fusing two or more rings, and the like in the specific example group G2.

The case where R₆₀₁ is bonded with the ring b, the case where R₆₀₂ is bonded with the ring a, and the case where R₆₀₂ is bonded with the ring c are also the same as the case described above.

In one embodiment, the ring a, the ring b and the ring c in the general formula (6) are independently a substituted or unsubstituted aromatic hydrocarbon ring having 6 to 50 ring carbon atoms.

In one embodiment, the ring a, the ring b and the ring c in the general formula (6) are independently a substituted or unsubstituted benzene ring or naphthalene ring.

In one embodiment, R₆₀₁ and R₆₀₂ in the general formula (6) are independently
a substituted or unsubstituted aryl group having 6 to 50 ring carbon atoms, or
a substituted or unsubstituted heterocyclic group having 5 to 50 ring atoms,
and preferably a substituted or unsubstituted aryl group having 6 to 50 ring carbon atoms.

In one embodiment, the compound represented by the general formula (6) is a compound represented by the following general formula (62).

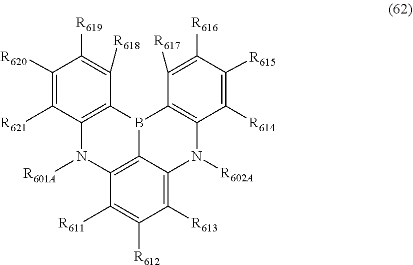

In the general formula (62),
R_{601A} forms a substituted or unsubstituted heterocycle by bonding with one or more groups selected from the group consisting of R₆₁₁ and R₆₂₁, or does not form the substituted or unsubstituted heterocycle;
R_{602A} forms a substituted or unsubstituted heterocycle by bonding with one or more groups selected from the group consisting of R₆₁₃ and R₆₁₄, or does not form the substituted or unsubstituted heterocycle;
R_{601A} and R_{602A} which do not form the substituted or unsubstituted heterocycle are independently
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms;
one or more sets of the adjacent two or more of R₆₁₁ to R₆₂₁
form a substituted or unsubstituted single ring by bonding with each other,
form a substituted or unsubstituted fused ring by bonding with each other, or
do not bond with each other;
R₆₁₁ to R₆₂₁ which do not form the substituted or unsubstituted heterocycle, which do not form the single ring and which do not form the fused ring are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a group represented by —N(R₉₀₆)(R₉₀₇),
a halogen atom,
a cyano group,
a nitro group,
a substituted or unsubstituted aryl group having 6 to 50 ring carbon atoms, or
a substituted or unsubstituted heterocyclic group having 5 to 50 ring atoms.

R_{601A} and R_{602A} in the general formula (62) are independently a group corresponding to R₆₀₁ and R₆₀₂ in the general formula (6).

For example, R_{601A} and R₆₁₁ may be bonded to form a two-ring-fused (or three or more-ring-fused) nitrogen-containing heterocycle in which a ring containing them and a benzene ring corresponding to the ring a are fused. Specific examples of the nitrogen-containing heterocycle include a compound corresponding to a heterocyclic group containing a nitrogen atom and fusing two or more rings, and the like in the specific example group G2. The case where R_{601A} and R₆₂₁ are bonded, the case where R_{602A} and R₆₁₃ are bonded, and the case where R_{602A} and R₆₁₄ are bonded are also the same as the case described above.
one or more sets of the adjacent two or more of R₆₁₁ to R₆₂₁ may
form a substituted or unsubstituted single ring by bonding with each other, or
form a substituted or unsubstituted fused ring by bonding with each other.

For example, R₆₁₁ and R₆₁₂ may be bonded in terms of a six-membered ring in which they are bonded to form a structure in which a benzene ring, an indole ring, a pyrrole ring, a benzofuran ring, a benzothiophene ring, or the like are fused, and the formed fused ring is a naphthalene ring, a carbazole ring, an indole ring, a dibenzofuran ring or a dibenzothiophene ring.

In one embodiment, R₆₁₁ to R₆₂₁ which do not form the ring are independently
a hydrogen atom,
a substituted or unsubstituted alkyl group having 1 to 50 carbon atoms,
a substituted or unsubstituted aryl group having 6 to 50 ring carbon atoms, or
a substituted or unsubstituted heterocyclic group having 5 to 50 ring atoms.

In one embodiment, R₆₁₁ to R₆₂₁ which do not form the ring are independently
a hydrogen atom,
a substituted or unsubstituted aryl group having 6 to 50 ring carbon atoms, or
a substituted or unsubstituted heterocyclic group having 5 to 50 ring atoms.

In one embodiment, R₆₁₁ to R₆₂₁ which do not form the ring are independently
a hydrogen atom, or
a substituted or unsubstituted alkyl group having 1 to 50 carbon atoms.

In one embodiment, R₆₁₁ to R₆₂₁ which do not form the ring are independently
a hydrogen atom, or
a substituted or unsubstituted alkyl group having 1 to 50 carbon atoms, and
at least one of R₆₁₁ to R₆₂₁ is a substituted or unsubstituted alkyl group having 1 to 50 carbon atoms.

In one embodiment, the compound represented by the general formula (62) is a compound represented by the following general formula (63).

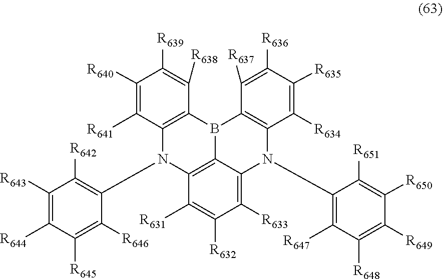

In the general formula (63),
R₆₃₁ forms a substituted or unsubstituted heterocycle by bonding with R₆₄₆, or does not form the substituted or unsubstituted heterocycle;
R₆₃₃ forms a substituted or unsubstituted heterocycle by bonding with R₆₄₇, or does not form the substituted or unsubstituted heterocycle;
R₆₃₄ forms a substituted or unsubstituted heterocycle by bonding with R₆₅₁, or does not form the substituted or unsubstituted heterocycle;
R₆₄₁ forms a substituted or unsubstituted heterocycle by bonding with R₆₄₂, or does not form the substituted or unsubstituted heterocycle;
one or more sets of the adjacent two or more of R₆₃₁ to R₆₅₁
form a substituted or unsubstituted single ring by bonding with each other,
form a substituted or unsubstituted fused ring by bonding with each other, or
do not bond with each other;
R₆₃₁ to R₆₅₁ which do not form the substituted or unsubstituted heterocycle, which do not form the single ring and which do not form the fused ring are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —S₁(R₉₀₀)(R₉₀₂)(R₉₀₃),
a group represented by —O—(R₉₀₄),
a group represented by —S—(R₉₀₅),
a group represented by —N(R₉₀₆)(R₉₀₇),
a halogen atom,
a cyano group,
a nitro group,
a substituted or unsubstituted aryl group having 6 to 50 ring carbon atoms, or
a substituted or unsubstituted heterocyclic group having 5 to 50 ring atoms.

R₆₃₁ may form a substituted or unsubstituted heterocycle by bonding with R₆₄₆. For example, R₆₃₁ and R₆₄₆ may be bonded to form a three or more-ring-fused nitrogen-containing heterocycle in which a benzene ring which R₆₄₆ is bonded with, a ring containing N and a benzene ring corresponding to the ring a are fused. Specific examples of the nitrogen-containing heterocycle include a compound corresponding to a heterocyclic group containing a nitrogen atom and fusing three or more rings, and the like in the specific example group G2. The case where R₆₃₃ and R₆₄₇ are bonded, the case where R₆₃₄ and R₆₅₁ are bonded, and the case where R₆₄₁ and R₆₄₂ are bonded are also the same as the case described above.

In one embodiment, R₆₃₁ to R₆₅₁ which do not form the ring are independently
a hydrogen atom,
a substituted or unsubstituted alkyl group having 1 to 50 carbon atoms,
a substituted or unsubstituted aryl group having 6 to 50 ring carbon atoms, or
a substituted or unsubstituted heterocyclic group having 5 to 50 ring atoms.

In one embodiment, R₆₃₁ to R₆₅₁ which do not form the ring are independently
a hydrogen atom,
a substituted or unsubstituted aryl group having 6 to 50 ring carbon atoms, or
a substituted or unsubstituted heterocyclic group having 5 to 50 ring atoms.

In one embodiment, R₆₃₁ to R₆₅₁ which do not form the ring are independently
a hydrogen atom, or
a substituted or unsubstituted alkyl group having 1 to 50 carbon atoms.

In one embodiment, R₆₃₁ to R₆₅₁ which do not form the ring are independently
a hydrogen atom, or
a substituted or unsubstituted alkyl group having 1 to 50 carbon atoms, and
at least one of R₆₃₁ to R₆₅₁ is a substituted or unsubstituted alkyl group having 1 to 50 carbon atoms.

In one embodiment, the compound represented by the general formula (63) is a compound represented by the following general formula (63A).

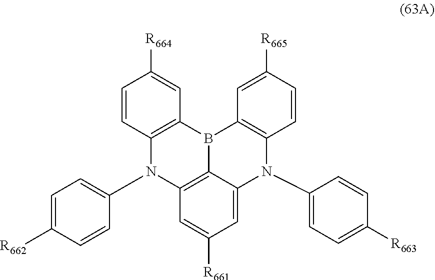

In the general formula (63A),
R₆₆₁ is
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms, or
a substituted or unsubstituted aryl group having 6 to 50 ring carbon atoms;
R₆₆₂ to R₆₆₅ are independently
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms, or
a substituted or unsubstituted aryl group having 6 to 50 ring carbon atoms.

In one embodiment, R₆₆₁ to R₆₆₅ are independently
a substituted or unsubstituted alkyl group having 1 to 50 carbon atoms, or
a substituted or unsubstituted aryl group having 6 to 50 ring carbon atoms.

In one embodiment, R₆₆₁ to R₆₆₅ are independently a substituted or unsubstituted alkyl group having 1 to 50 carbon atoms.

In one embodiment, the compound represented by the general formula (63) is a compound represented by the following general formula (63B).

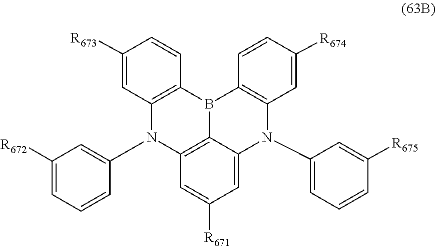

In the general formula (63B),
R₆₇₁ and R₆₇₂ are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —N(R₉₀₆)(R₉₀₇), or
a substituted or unsubstituted aryl group having 6 to 50 ring carbon atoms; and
R₆₇₃ to R₆₇₅ are independently
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —N(R₉₀₆)(R₉₀₇), or
a substituted or unsubstituted aryl group having 6 to 50 ring carbon atoms.

In one embodiment, the compound represented by the general formula (63) is a compound represented by the following general formula (63B′).

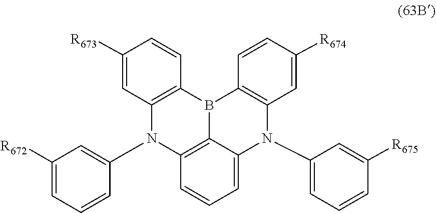

In the general formula (63B′), R₆₇₂ to R₆₇₅ are independently the same as defined for R₆₇₂ to R₆₇₅ in the general formula (63B).

In one embodiment, at least one of R₆₇₁ to R₆₇₅ is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a group represented by —N(R₉₀₆)(R₉₀₇), or
a substituted or unsubstituted aryl group having 6 to 50 ring carbon atoms.

In one embodiment,
R₆₇₂ is
a hydrogen atom,
a substituted or unsubstituted alkyl group having 1 to 50 carbon atoms,
a group represented by —N(R₉₀₆)(R₉₀₇), or
a substituted or unsubstituted aryl group having 6 to 50 ring carbon atoms; and
R₆₇₁ and R₆₇₃ to R₆₇₅ are independently
a substituted or unsubstituted alkyl group having 1 to 50 carbon atoms,
a group represented by —N(R₉₀₆)(R₉₀₇), or
a substituted or unsubstituted aryl group having 6 to 50 ring carbon atoms.

In one embodiment, the compound represented by the general formula (63) is a compound represented by the following general formula (63C).

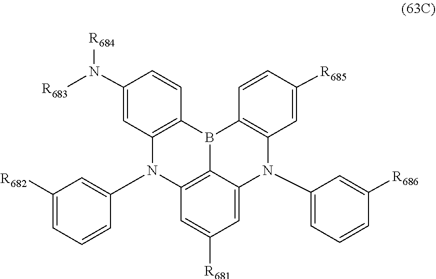

In the general formula (63C),
R₆₈₁ and R₆₈₂ are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms, or
a substituted or unsubstituted aryl group having 6 to 50 ring carbon atoms;
R₆₈₃ to R₆₈₆ are independently
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms, or
a substituted or unsubstituted aryl group having 6 to 50 ring carbon atoms.

In one embodiment, the compound represented by the general formula (63) is a compound represented by the following general formula (63C′).

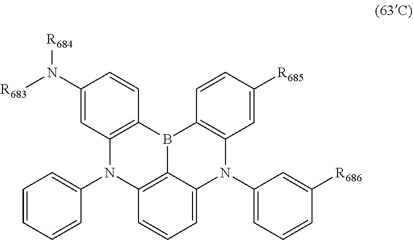

In the general formula (63C′), R₆₃₃ to R₆₈₆ are independently the same as defined for R₆₃₃ to R₆₈₆ in the general formula (63C).

In one embodiment, R₆₈₁ to R₆₈₆ are independently
a substituted or unsubstituted alkyl group having 1 to 50 carbon atoms, or
a substituted or unsubstituted aryl group having 6 to 50 ring carbon atoms.

In one embodiment, R₆₈₁ to R₆₈₆ are independently a substituted or unsubstituted aryl group having 6 to 50 ring carbon atoms.

In the compound represented by the general formula (6), first, the ring a, the ring b and the ring c can be bonded via a linking group (a group containing N—R₆₀₁ and a group containing N—R₆₀₂) to produce an intermediate (first reaction), and the ring a, the ring b and the ring c can be bonded via a linking group (a group containing a boron atom) to produce the final product (second reaction). An amination reaction such as Bachburt-Hartwig reaction can be used for the first reaction. A tandem hetero-Friedel-Crafts reaction, and the like can be used for the second reaction.

The compound represented by the general formula (6) can be produced by a known method.

### (Specific Examples of Compound Represented by the General Formula (6))

Specific examples of the compound represented by the general formula (6) include the following compounds. Here, the compound represented by the general formula (6) is not limited to the following specific examples.

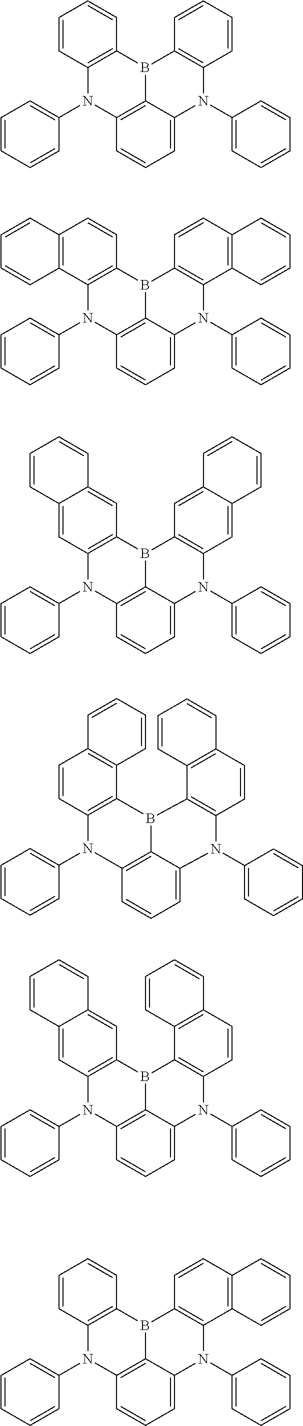

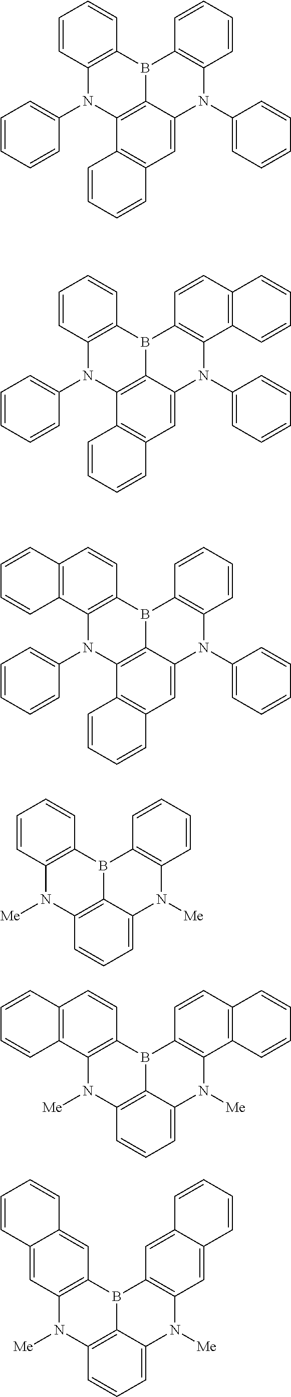

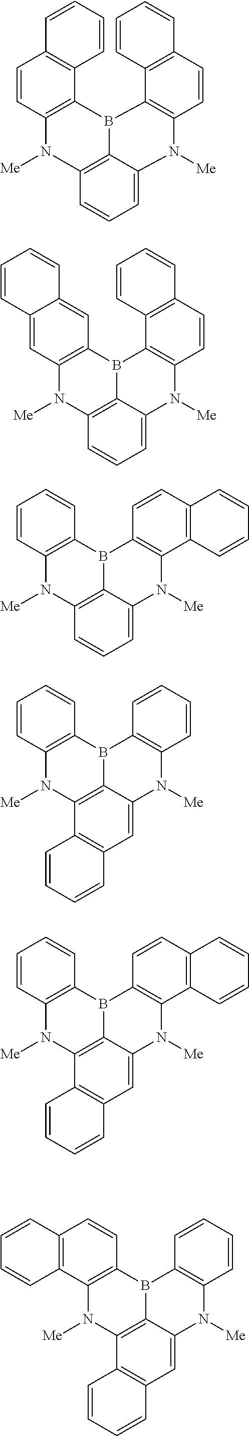

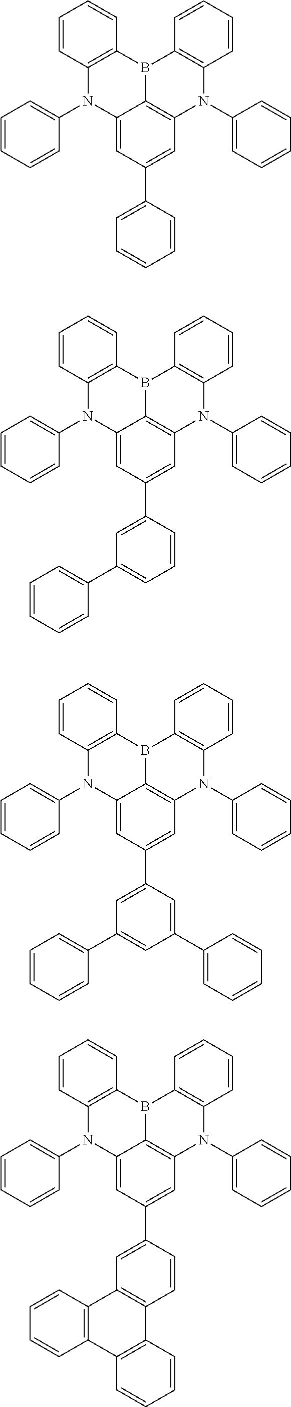

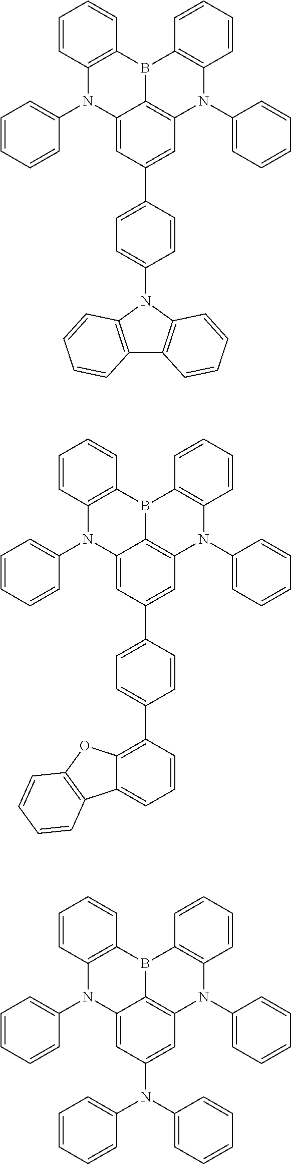

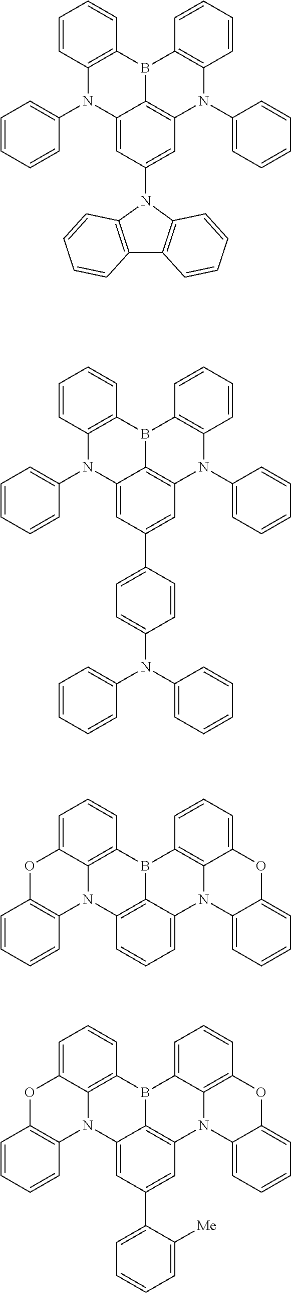

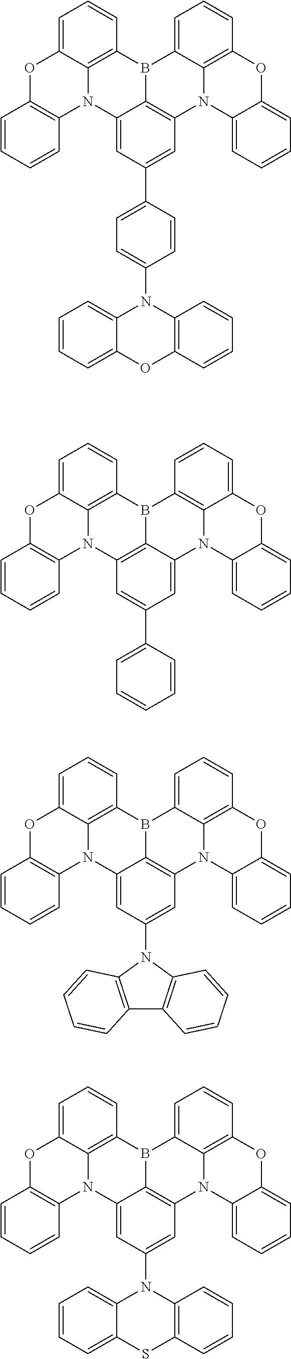

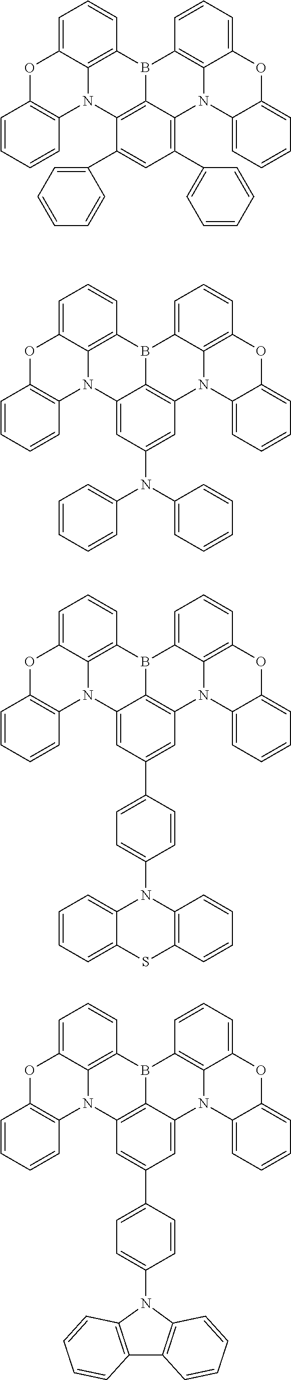

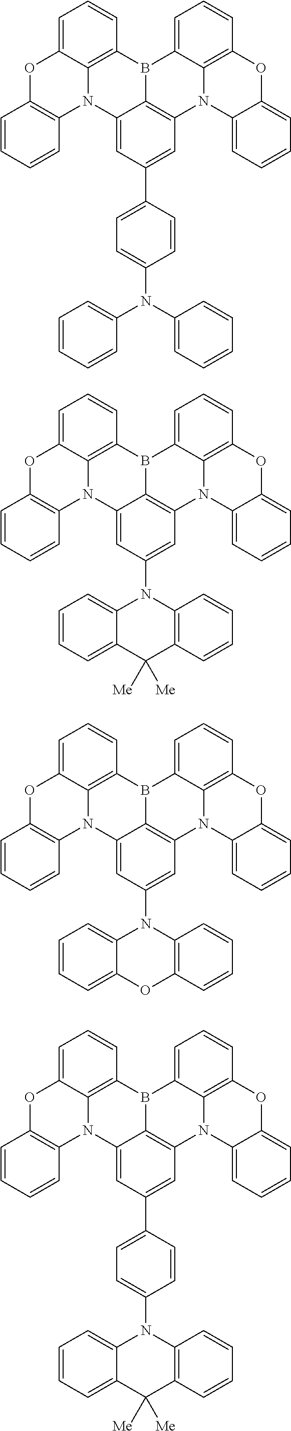

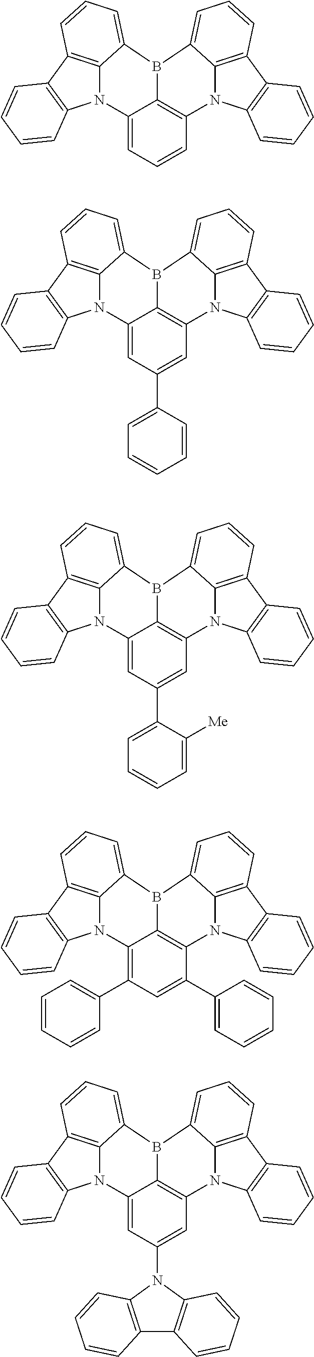

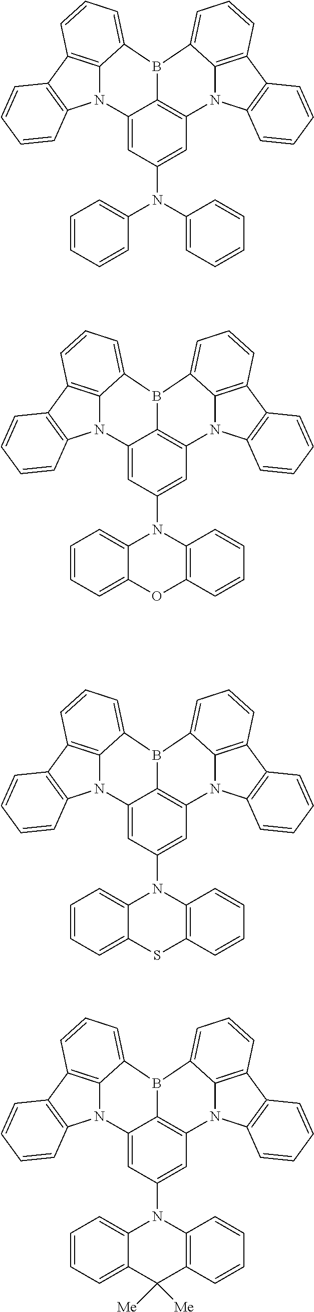

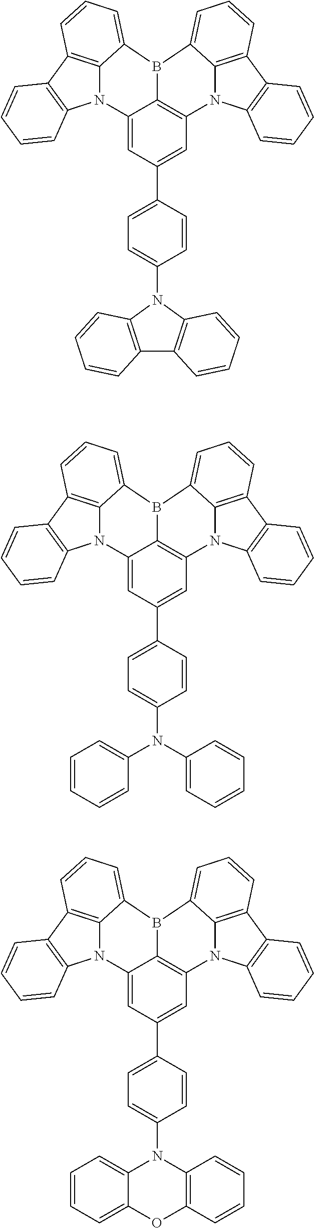

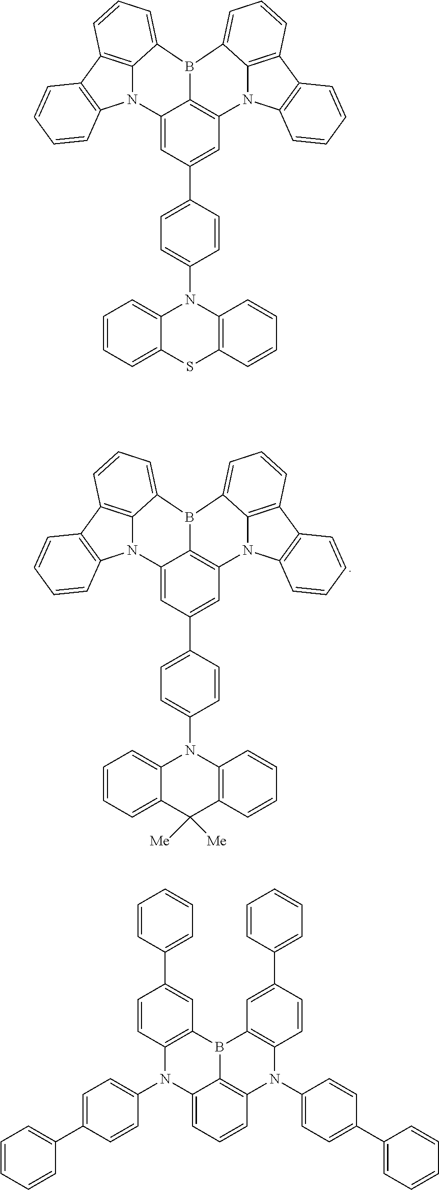

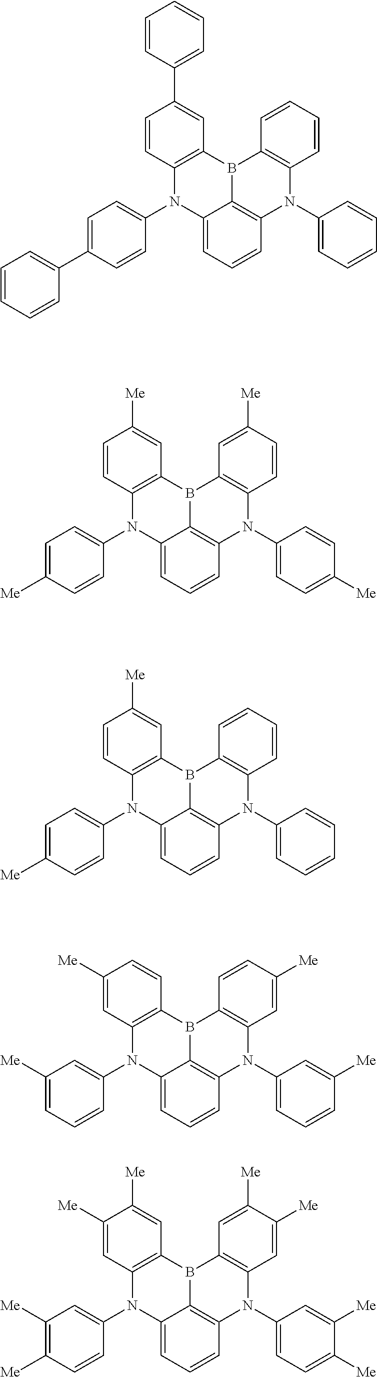

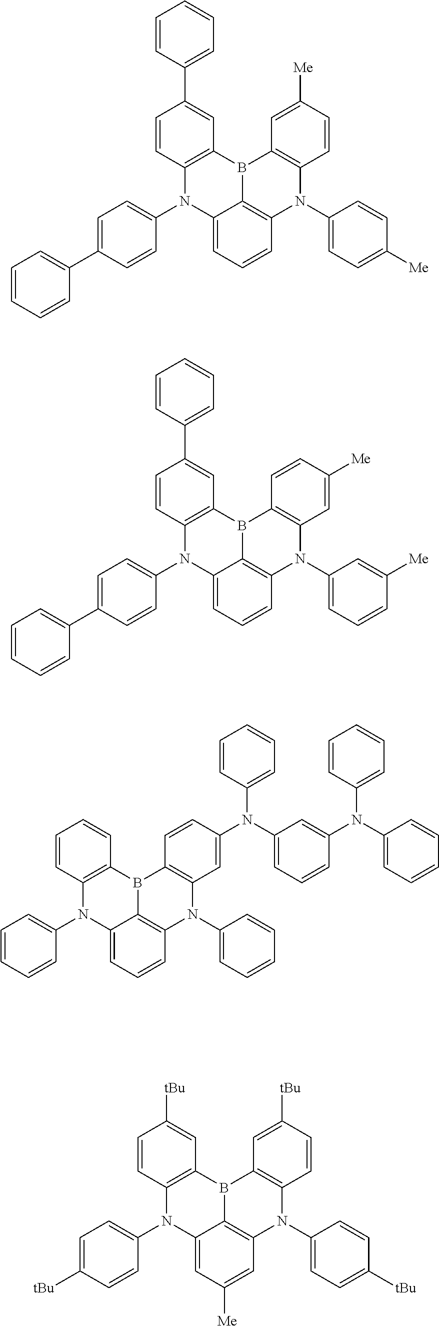

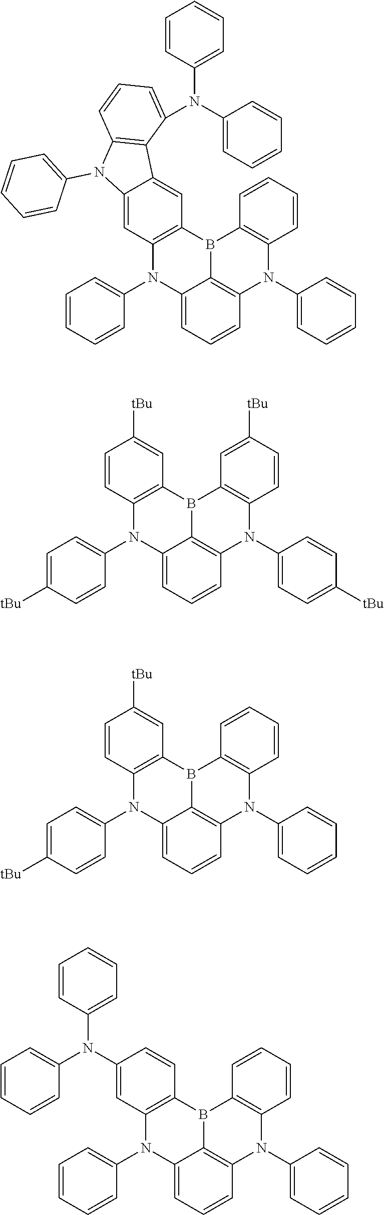

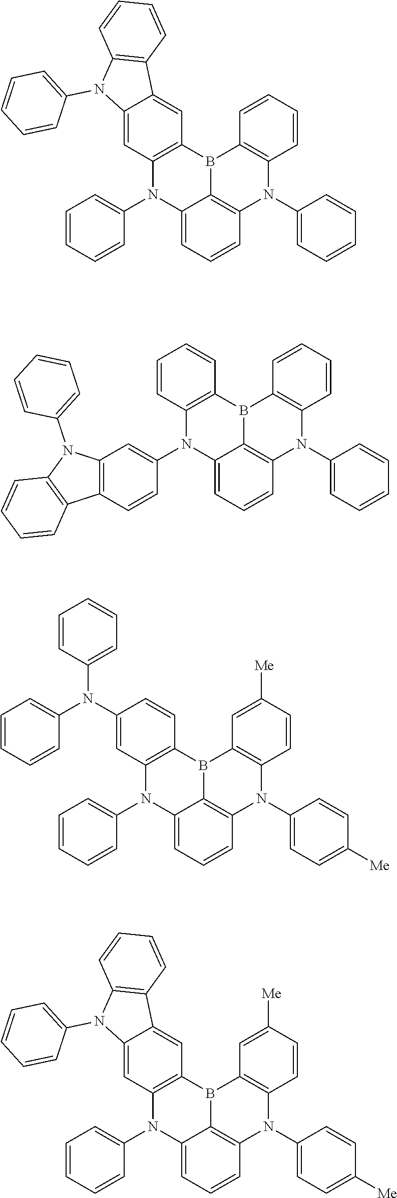

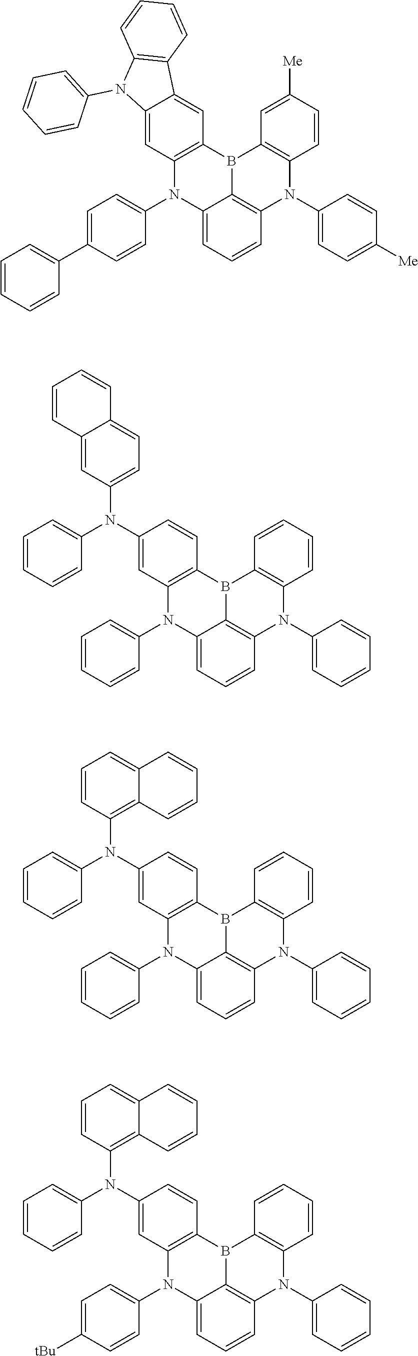

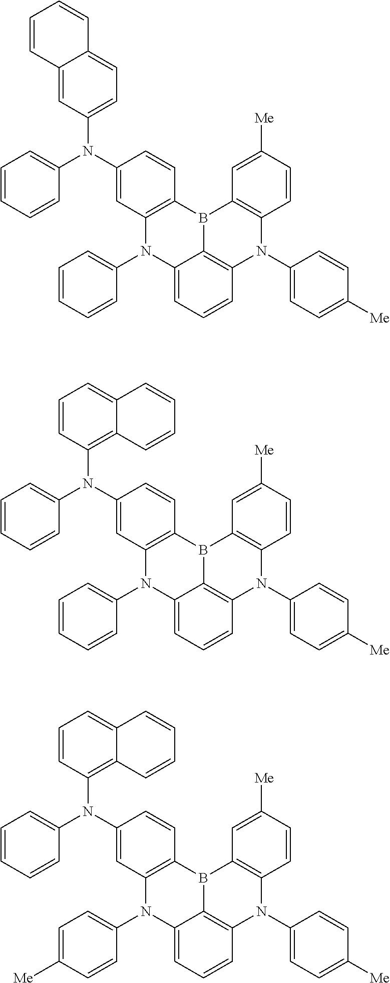

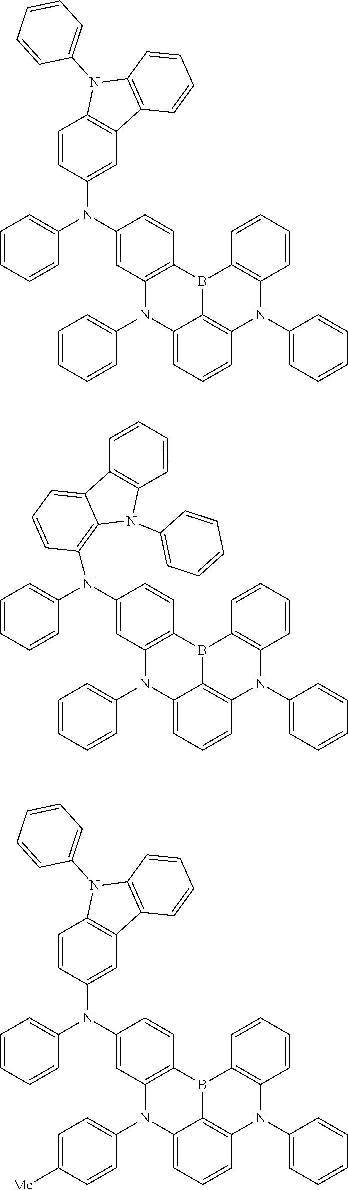

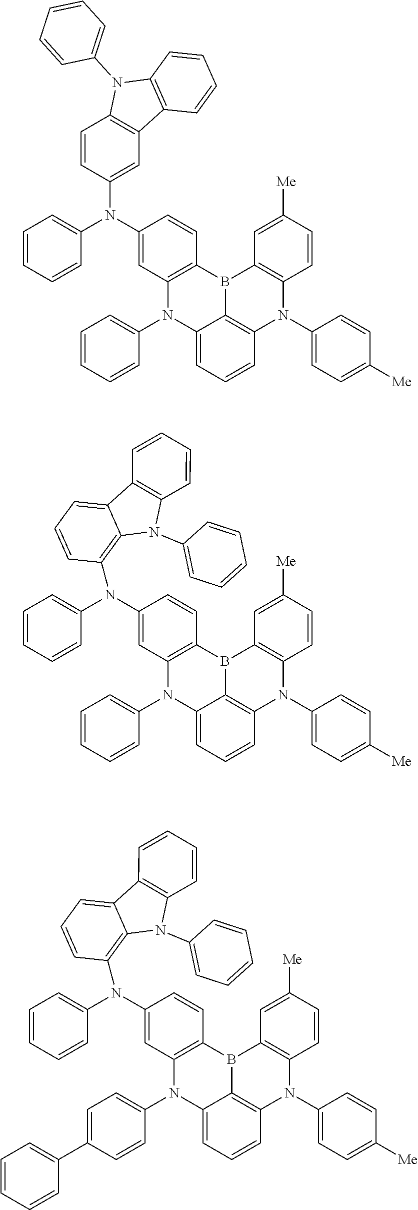

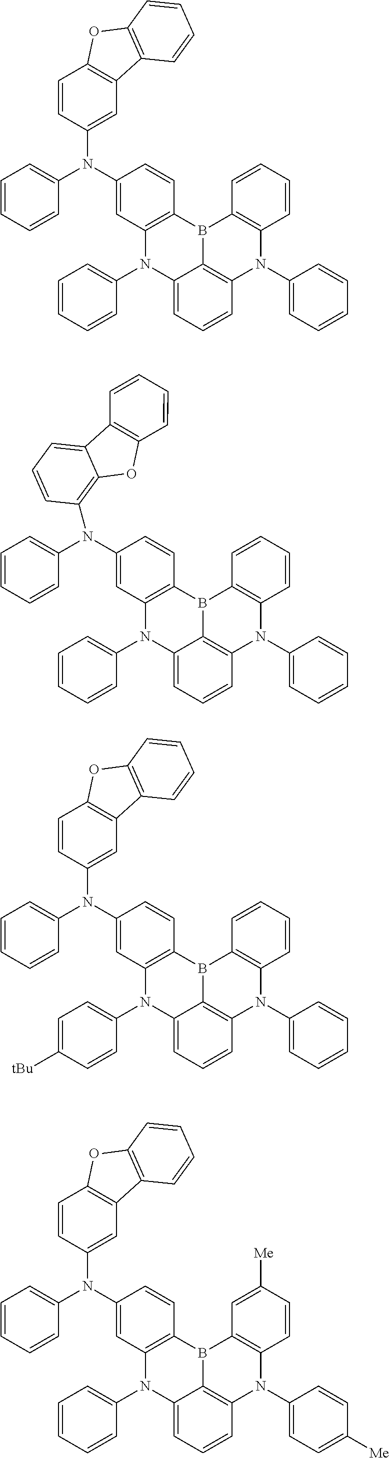

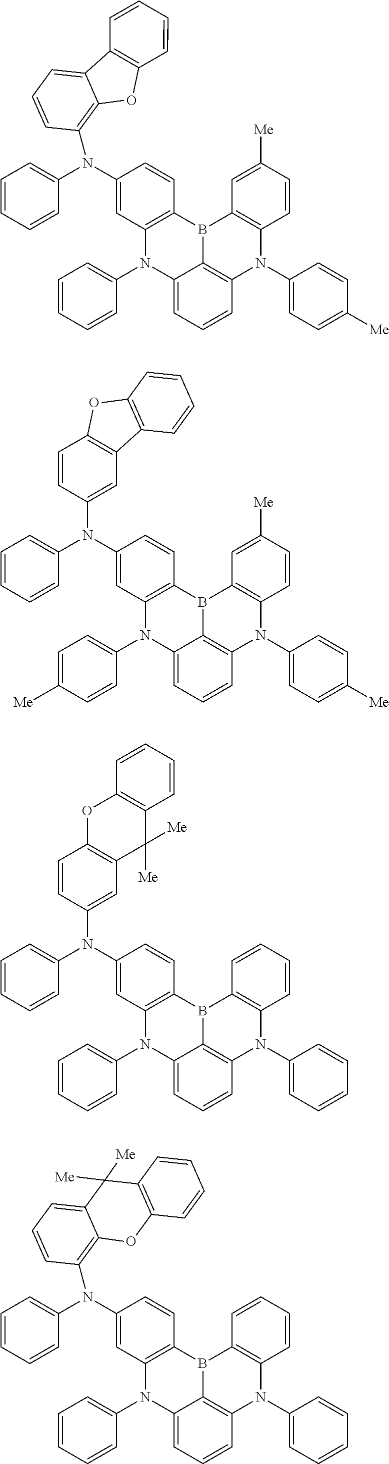

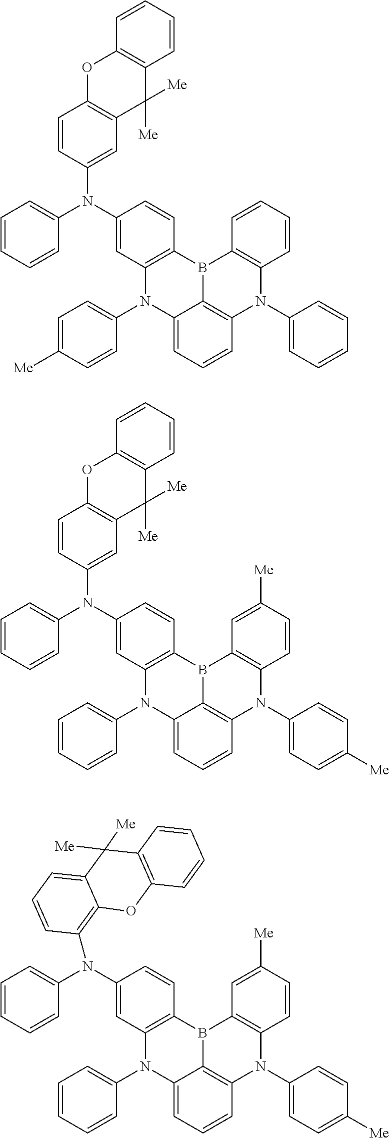

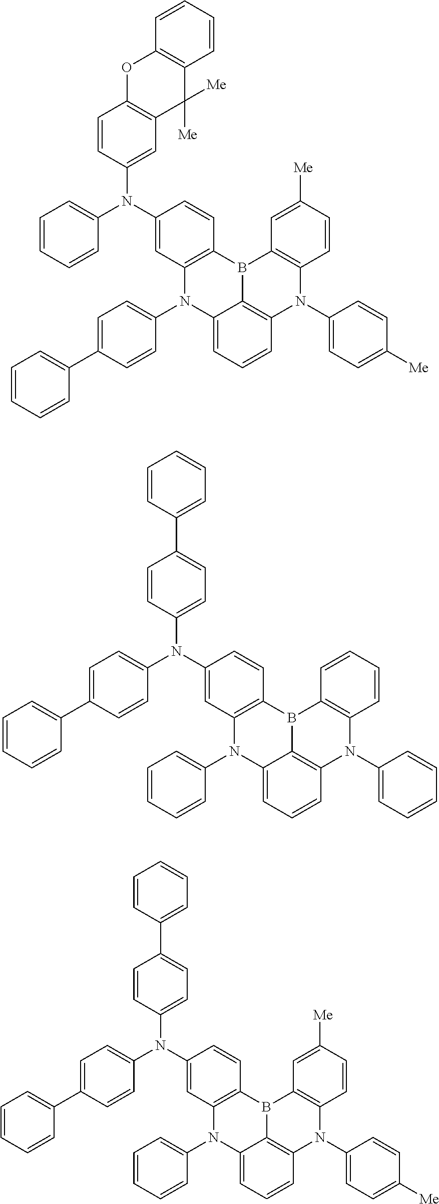

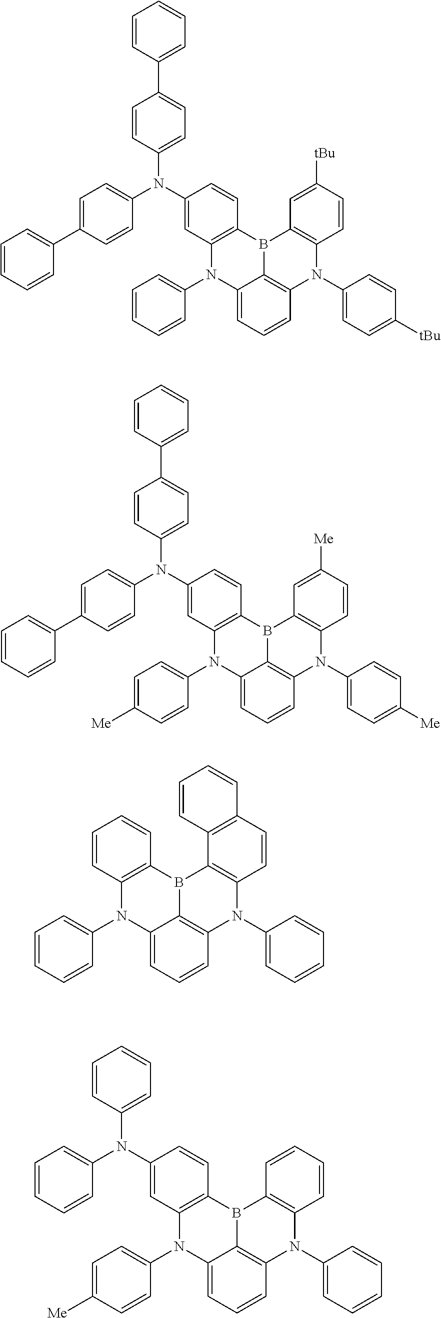

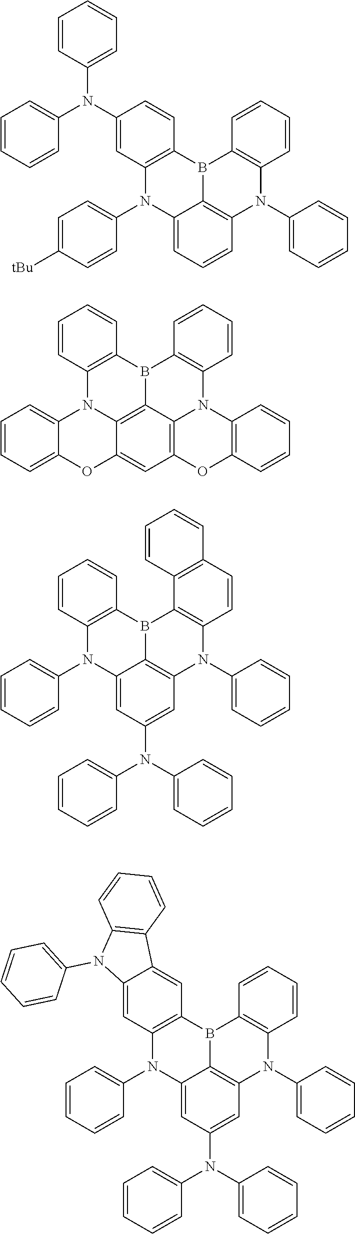

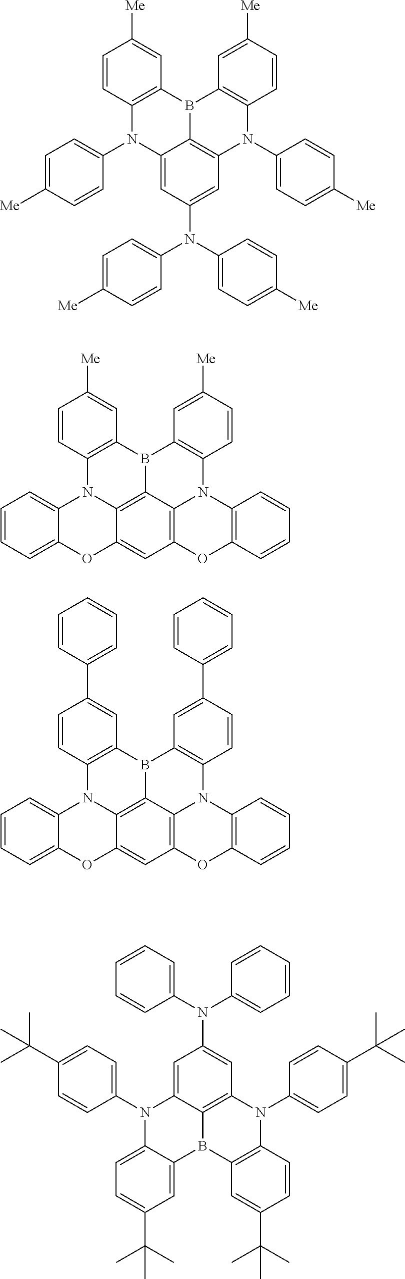

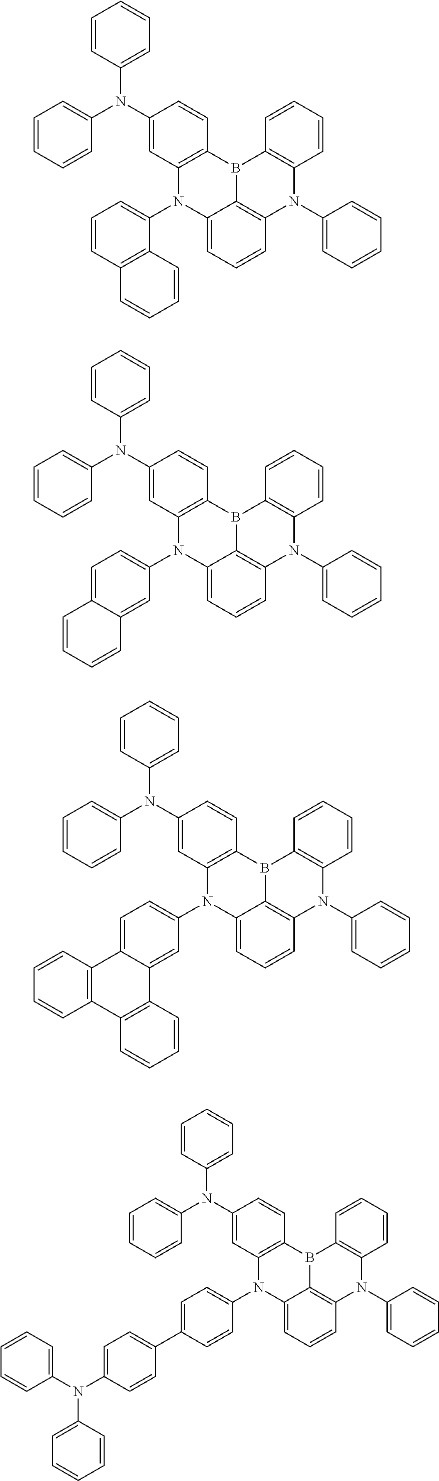

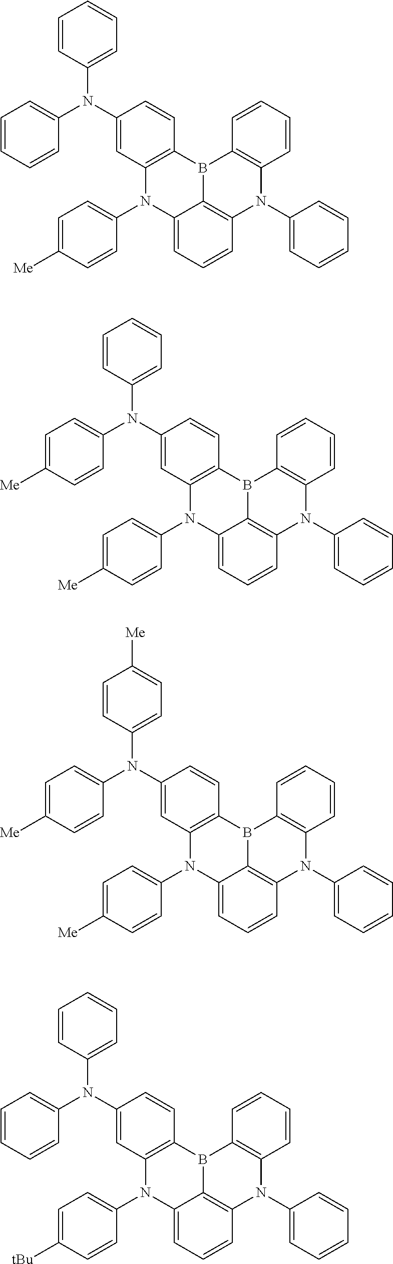

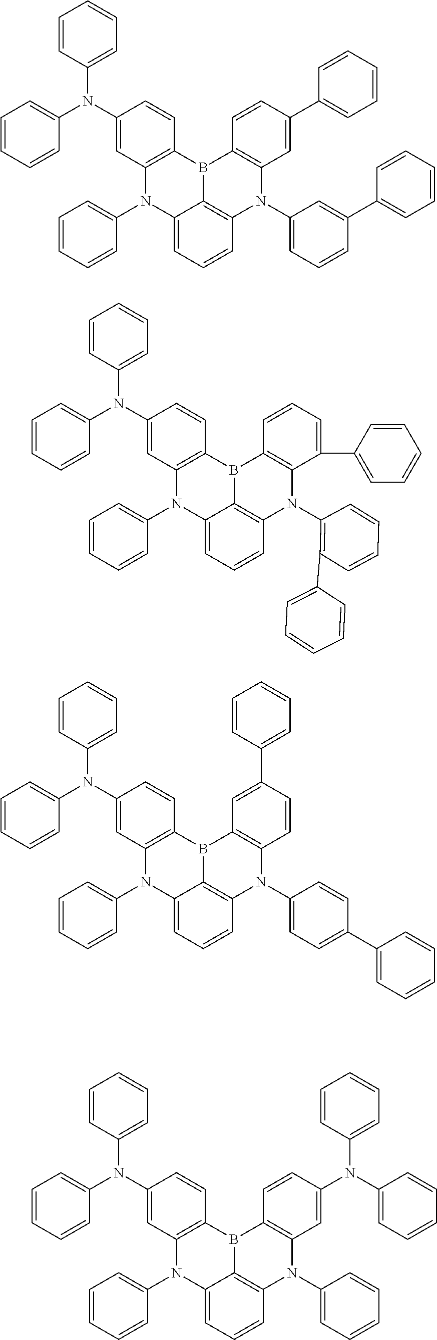

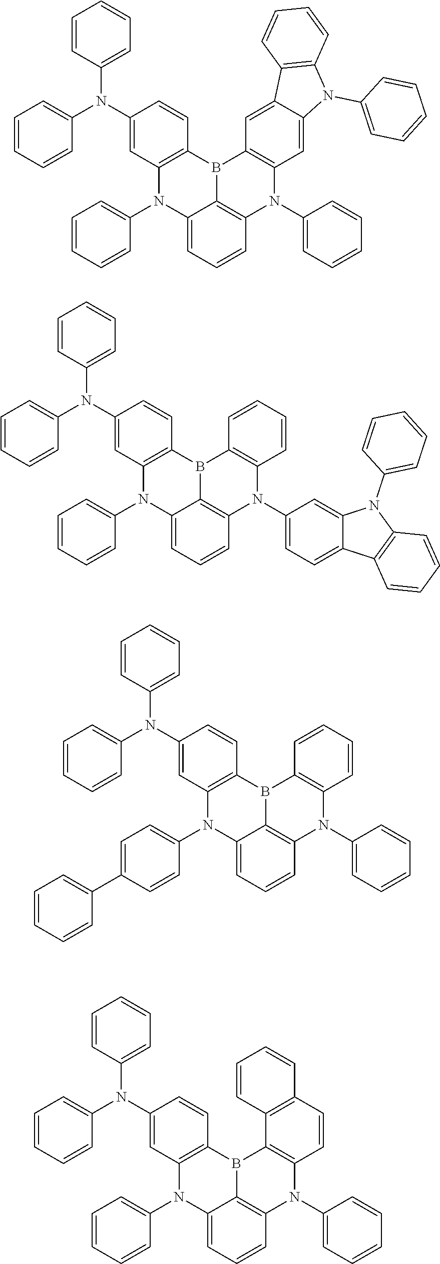

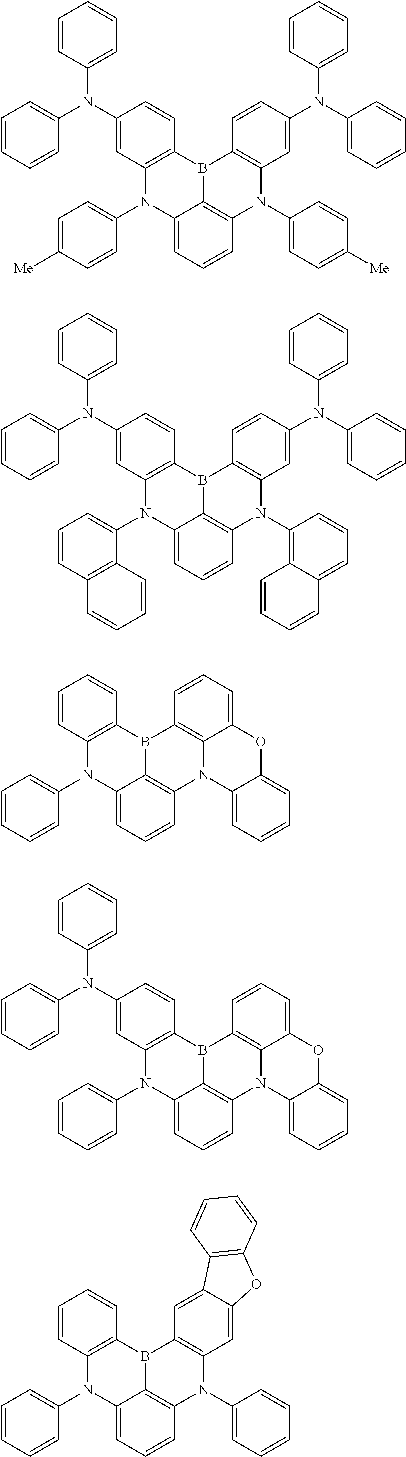

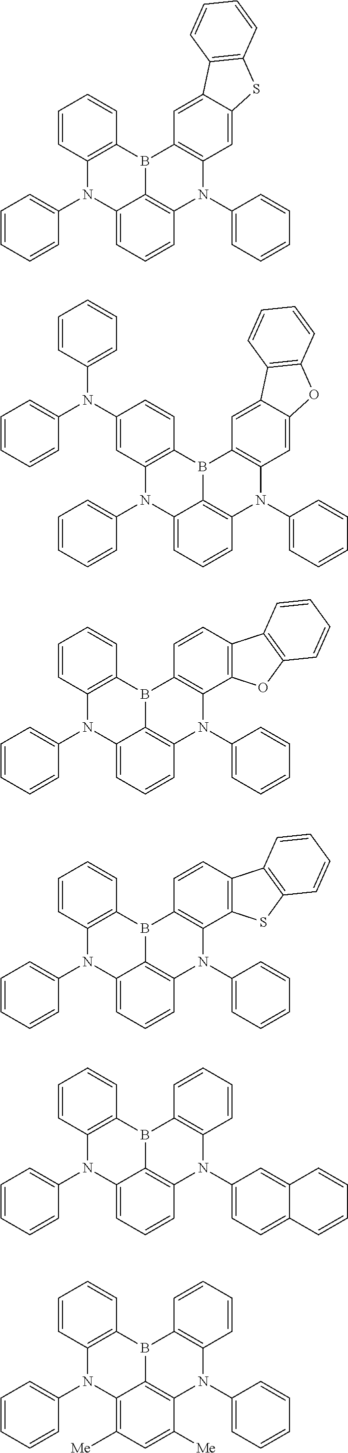

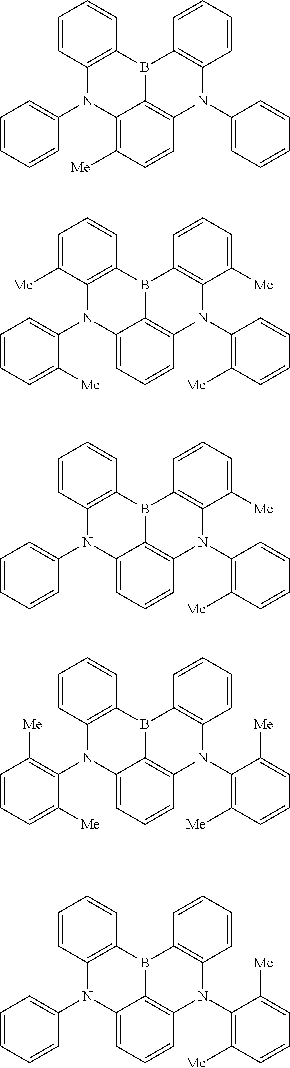

In the organic EL device according to the second embodiment, the singlet energy S, (H2) of the second host material and the singlet energy S₁ (D2) of the second dopant material preferably satisfy the relationship of the following numerical formula (Numerical formula 20).

S₁(H2)>S₁(D2) (Numerical formula 20)

When the second host material and the second dopant material satisfy the relationship of the numerical formula (Numerical formula 20), a singlet exciton generated on the second host material is likely to transfer energy from the second host material to the second dopant material to contribute to the emission (preferably fluorescent emission) of the second dopant material.

In the organic EL device according to the second embodiment, the triplet energy T₁ (H2) of the second host material and the triplet energy T₁ (D2) of the second dopant material preferably satisfy the relationship of the following numerical formula (Numerical formula 20A).

T₁(D2)>T₁(H2) (Numerical formula 20A)

When the second host material and the second dopant material satisfy the relationship of the numerical formula (Numerical formula 20A), since a triplet exciton generated in the second emitting layer is transferred on the second host material rather than on the second dopant material having a higher triplet energy, it is likely to be transferred to the first emitting layer.

In the organic EL device according to the second embodiment, the second dopant material is preferably included in the second emitting layer in an amount of 0.5% by mass or more based on the total mass of the second emitting layer, more preferably more than 1.1% by mass based on the total mass of the second emitting layer, still more preferably 1.2% by mass or more based on the total mass of the second emitting layer, and most preferably 1.5% by mass or more based on the total mass of the second emitting layer.

The second dopant material is preferably included in the second emitting layer in an amount of 10% by mass or less based on the total mass of the second emitting layer, more preferably 7% by mass or less based on the total mass of the second emitting layer, and still more preferably 5% by mass or less based on the total mass of the second emitting layer.

In the organic EL device according to the second embodiment, the second host material is preferably included in the second emitting layer in an amount of 60% by mass or more based on the total mass of the second emitting layer, more preferably 70% by mass or more based on the total mass of the second emitting layer, still more preferably 80% by mass or more based on the total mass of the second emitting layer, still more preferably 90% by mass or more based on the total mass of the second emitting layer, and most preferably 95% by mass or more based on the total mass of the second emitting layer.

The second host material is preferably included in the second emitting layer in an amount of 99% by mass or less based on the total mass of the second emitting layer.

Here, when the second host material and the second dopant material are included in the second emitting layer, the upper limit of the total amount of the second host material and the second dopant material is 100% by mass.

In the second embodiment, it is excluded that a material other than the second host material and the second dopant material is included in the first emitting layer.

The second emitting layer may include one kind of the second host material alone, or may include two or more kinds thereof in combination. The second emitting layer may include one kind of the second dopant material alone, or may include two or more kinds thereof in combination.

In the organic EL device according to the second embodiment, the second emitting layer preferably has the thickness of 3 nm or more, and more preferably 5 nm or more. When the second emitting layer has the thickness of 3 nm or more, the thickness in the second emitting layer is sufficient to cause recombination of a hole and an electron.

In the organic EL device according to the second embodiment, the second emitting layer preferably has the thickness of 15 nm or less, and more preferably 10 nm or less. When the second emitting layer has the thickness of 15 nm or less, the thickness is thin enough for a triplet exciton to migrate to the first emitting layer.

In the organic EL device according to the second embodiment, the second emitting layer more preferably has the thickness of 3 nm or more and 15 nm or less.

### (Other Layers of Organic EL Device)

The organic EL device according to the second embodiment may include one or more organic layers in addition to the first emitting layer and the second emitting layer. As the organic layer, for example, at least any one layer selected from the group consisting of the hole-injecting layer, the hole-transporting layer, the emitting layer, the electron-injecting layer, the electron-transporting layer, the hole-barrier layer and the electron-barrier layer can be given.

The organic EL device according to a second embodiment may include, for example, the anode, the second emitting layer, the first emitting layer, the cathode in this order, or may include the anode, the first emitting layer, the second emitting layer, the cathode by reversing the order of the second emitting layer and the first emitting layer. In any order of the first emitting layer and the second emitting layer, when a combination of the host materials satisfying the relationship of the numerical formula (Numerical formula 1) is selected and the first compound (the compound represented by the general formula (1)) according to the first embodiment is included in the first emitting layer, effects (effect extending the device lifetime and effect improving the luminous efficiency thereof) are expected by using a stacking configuration for the emitting layer described above.

In the organic EL device according to the second embodiment, it may be configured only using the first emitting layer and the second emitting layer, and for example, it may further include at least any one layer selected from the group consisting of the hole-injecting layer, the hole-transporting layer, the electron-injecting layer, the electron-transporting layer, the hole-barrier layer, the electron-barrier layer, and the like.

The organic EL device according to the second embodiment preferably includes the first emitting layer arranged between the anode and the cathode, and includes the second emitting layer arranged between the first emitting layer and the anode.

The organic EL device according to the present embodiment also preferably includes the first emitting layer arranged between the anode and the cathode, and includes the second emitting layer arranged between the first emitting layer and the cathode.

In the organic EL device according to the second embodiment, the hole-transporting layer is preferably included between the emitting layer and the anode.

In the organic EL device according to the second embodiment, the electron-transporting layer is preferably included between the emitting layer and the cathode.

### (Third Emitting Layer)

The organic EL device according to the second embodiment may further include a third emitting layer.

The third emitting layer includes a third host material, wherein the first host material, the second host material and the third host material are different from each other, the third emitting layer includes at least third dopant material, wherein the first dopant material, the second dopant material and the third dopant material are the same as or different from each other, and the triplet energy T₁ (H2) of the second host material and the triplet energy T₁ (H3) of the third host material preferably satisfy the relationship of the following numerical formula (Numerical formula 5).

T₁(H2)>T₁(H3) (Numerical formula 5)

The third dopant material is preferably a compound in which the maximum peak wavelength represents an emission of 500 nm or less, and more preferably a compound in which the maximum peak wavelength represents a fluorescent emission of 500 nm or less.

When the organic EL device according to the second embodiment includes the third emitting layer, the triplet energy T₁ (H1) of the first host material and the triplet energy T₁ (H3) of the third host material preferably satisfy the relationship of the following numerical formula (Numerical formula 6.

T₁(H1)>T₁(H3) (Numerical formula 6)

The third host material is not particularly limited, and for example, a host material described as the first host material and the second host material in the embodiment can be used.

The third dopant material is not particularly limited, and for example, a dopant material described as the first dopant material and the second dopant material in the embodiment can be used.

In the organic EL device according to the second embodiment, the first emitting layer and the second emitting layer are preferably directly in contact with each other.

In the present specification, a layer structure of the expression “the first emitting layer and the second emitting layer are directly in contact with each other” can include, for example, any one aspect of the following aspects (LS1), (LS2) and (LS3).

Aspect (LS1) in which a region where both of the first host material and the second host material are mixed is generated in the process through depositing compounds for the first emitting layer and depositing compounds for the second emitting layer, and the region is present at the interface between the first emitting layer and the second emitting layer

Aspect (LS2) in which in the case where the first emitting layer and the second emitting layer include an emitting compound (dopant material), a region where the first host material, the second host material and emitting compound are mixed is generated in the process through depositing compounds for the first emitting layer and depositing compounds for the second emitting layer, and the region is present at the interface between the first emitting layer and the second emitting layer

Aspect (LS3) in which in the case where the first emitting layer and the second emitting layer include an emitting compound, a region composed of the emitting compound, a region composed of the first host material, or a region composed of the second host material is generated in the process through depositing compounds for the first emitting layer and depositing compounds for the second emitting layer, and the region is present at the interface between the first emitting layer and the second emitting layer

When the organic EL device according to the second embodiment includes the third emitting layer, it is preferable that the first emitting layer and the second emitting layer be directly in contact with each other, and the first emitting layer and the third emitting layer be directly in contact with each other.

In the present specification, a layer structure of the expression “the first emitting layer and the third emitting layer are directly in contact with each other” can include, for example, any one aspect of the following aspects (LS4), (LS5) and (LS6).

Aspect (LS4) in which a region where both of the first host material and the third host material are mixed is generated in the process through depositing compounds for the first emitting layer and depositing compounds for the third emitting layer, and the region is present at the interface between the first emitting layer and the third emitting layer

Aspect (LS5) in which in the case where the first emitting layer and the third emitting layer include an emitting compound (dopant material), a region where the first host material, the third host material and emitting compound are mixed is generated in the process through depositing compounds for the first emitting layer and depositing compounds for the third emitting layer, and the region is present at the interface between the first emitting layer and the third emitting layer

Aspect (LS6) in which in the case where the first emitting layer and the third emitting layer include an emitting compound, a region composed of the emitting compound, a region composed of the first host material, or a region composed of the third host material is generated in the process through depositing compounds for the first emitting layer and depositing compounds for the third emitting layer, and the region is present at the interface between the first emitting layer and the third emitting layer

When the organic EL device according to the second embodiment includes an intermediate layer, the intermediate layer is preferably arranged between the first emitting layer and the second emitting layer.

The intermediate layer is preferably a non-doped layer. The intermediate layer is preferably a layer not containing the emitting compound (dopant material). It is preferable that the intermediate layer do not include a metal atom.

The intermediate layer includes an intermediate layer material. It is preferable that the intermediate layer material is not the emitting compound.

The intermediate layer material is not particularly limited, and it is preferably a material other than the emitting compound.

As the intermediate layer material, for example, 1) a heterocyclic compound such as an oxadiazole derivative, a benzimidazole derivative, and a phenanthroline derivative, 2) a fused aromatic compound such as a carbazole derivative, an anthracene derivative, a phenanthrene derivative, a pyrene derivative, and a chrysene derivative, and 3) an aromatic amine compound such as a triarylamine derivative and a fused polycyclic aromatic amine derivative can be given.

The intermediate layer material may be one of the first host material included in the first emitting layer and the second host material included in the second emitting layer, or may be both of the materials.

When the intermediate layer includes a plurality of intermediate layer materials, the amount of each of the intermediate layer materials is preferably 10% by mass or more based on the total mass of the intermediate layer.

The intermediate layer material is preferably included in the intermediate layer in an amount of 60% by mass or more based on the total mass of the intermediate layer, more preferably 70% by mass or more based on the total mass of the intermediate layer, still more preferably 80% by mass or more based on the total mass of the intermediate layer, still more preferably 90% by mass or more based on the total mass of the intermediate layer, and most preferably 95% by mass or more based on the total mass of the intermediate layer.

The intermediate layer may include one kind of the intermediate layer material alone, or may include two or more kinds thereof in combination.

When the intermediate layer includes two or more kinds of intermediate layer materials, the upper limit of the total amount of the two or more kinds of intermediate layer materials is 100% by mass.

In the second embodiment, it is excluded that a material other than the intermediate layer material is included in the intermediate layer.

The intermediate layer may be constituted of a single layer or of two or more stacked layers.

The thickness of the intermediate layer is not particularly limited, and the thickness per layer is preferably 3 nm or more and 15 nm or less, and more preferably 5 nm or more and 10 nm or less.

Configurations of the organic EL device will be further described. They are configurations common to the first embodiment and the second embodiment. Hereinafter, the description for the symbol is frequently abbreviated.

As the representative device configuration of the organic EL device of the present invention, the following structures may be given:
(1) an anode/an emitting layer/a cathode,
(2) an anode/a hole-injecting layer/an emitting layer/a cathode,
(3) an anode/an emitting layer/an electron-injecting-transporting layer/a cathode,
(4) an anode/a hole-injecting layer/an emitting layer/an electron-injecting-transporting layer / a cathode,
(5) an anode/an organic semiconductor layer/an emitting layer/a cathode,
(6) an anode/an organic semiconductor layer/an electron-barrier layer/an emitting layer/a cathode,
(7) an anode/an organic semiconductor layer/an emitting layer/an adhesion improving layer / a cathode,
(8) an anode/a hole-injecting-transporting layer/an emitting layer/an electron-injecting-transporting layer/a cathode,
(9) an anode/an insulating layer/an emitting layer/an insulating layer/a cathode,
(10) an anode/an inorganic semiconductor layer/an insulating layer/an emitting layer/an insulating layer/a cathode,
(11) an anode/an organic semiconductor layer/an insulating layer/an emitting layer/an insulating layer/a cathode,
(12) an anode/an insulating layer/a hole-injecting-transporting layer/an emitting layer/an insulating layer/a cathode, and
(13) an anode/an insulating layer/a hole-injecting-transporting layer/an emitting layer/an electron-injecting-transporting layer/a cathode.

Among the above-described structures, the configuration of (8) is preferably used, but the device configuration of the organic EL device is not limited thereto.

In the present specification, the term “hole-injecting-transporting layer” means “at least one of the hole-injecting layer and the hole-transporting layer”, and the term “electron-injecting-transporting layer” means “at least one of the electron-injecting layer and the electron-transporting layer”.

Members which can be used in the organic EL device according to an aspect of the present invention, materials for forming each layer, other than the above-mentioned compounds, and the like, will be described below.

### (Substrate)

The substrate is used as a support of an emitting device. As the substrate, glass, quartz, plastic or the like can be used, for example. Further, a flexible substrate may be used. The term “flexible substrate” means a bendable (flexible) substrate, and specific examples thereof include a plastic substrate formed of polycarbonate, polyvinyl chloride or the like.

### (Anode)

For the anode formed on the substrate, metals, alloys, electrically conductive compounds, mixtures thereof, and the like, which have large work function (specifically 4.0 eV or more) are preferably used. Specific examples thereof include indium oxide-tin oxide (ITO: Indium Tin Oxide), indium oxide-tin oxide containing silicon or silicon oxide, indium oxide-zinc oxide, tungsten oxide, indium oxide containing zinc oxide, graphene, and the like. In addition thereto, specific examples thereof include gold (Au), platinum (Pt), a nitride of a metallic material (for example, titanium nitride), or the like.

### (Hole-Injecting Layer)

The hole-injecting layer is a layer containing a substance having high hole-injecting property. As the substance having high hole-injecting property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, a polymer compound (oligomers, dendrimers, polymers, and the like), or the like can be given.

### (Hole-Transporting Layer)

The hole-transporting layer is a layer containing a substance having high hole-transporting property. For the hole-transporting layer, an aromatic amine compound, a carbazole derivative, an anthracene derivative, or the like can be used. A polymer compound such as poly(N-vinylcarbazole) (abbreviation: PVK) and poly(4-vinyltriphenylamine) (abbreviation: PVTPA) can also be used. Provided that a substance other than the above-described substances may be used as long as the substance has higher hole-transporting property than electron-transporting property. The layer containing the substance having high hole-transporting property may be not only a single layer, but also layers in which two or more layers formed of the above-described substances are stacked.

### (Emitting Layer)

The emitting layer of the organic EL device may be any of an emitting layer of a fluorescent type, an emitting layer of a phosphorescent type or an emitting layer using Thermally Activated Delayed Fluorescence mechanism.

Further, the organic EL device may be a monochromatic emitting device of a fluorescent type, of a phosphorescent type and using Thermally Activated Delayed Fluorescence mechanism, or may be a white emitting device of a hybrid type of these, and it may be a simple type including a single emitting unit, or may be a tandem type including a plurality of emitting units. Here, the “emitting unit” refers to the smallest unit which includes one or more organic layers, in which one of them is an emitting layer, and which can emit light by recombination of injected holes and electrons.

The “emitting layer” described in the present specification is an organic layer having an emitting function. The emitting layer is, for example, a phosphorescent emitting layer, a fluorescent emitting layer, or the like, and may be a single layer or a plurality of layers.

### (Guest (Dopant) Material of Emitting Layer)

The emitting layer is a layer containing a substance having high luminous property, and various materials can be used in addition to the material (the compound represented by the formula (1)) used in the present invention described above. For example, as the substance having high emitting property, a fluorescent compound which emits fluorescence or a phosphorescent compound which emits phosphorescence can be used. The fluorescent compound is a compound which can emit from a singlet excited state, and the phosphorescent compound is a compound which can emit from a triplet excited state.

As a blue fluorescent emitting material which can be used for the emitting layer, pyrene derivatives, styrylamine derivatives, chrysene derivatives, fluoranthene derivatives, fluorene derivatives, diamine derivatives, triarylamine derivatives, and the like can be used. As a green fluorescent emitting material which can be used for the emitting layer, aromatic amine derivatives and the like can be used. As a red fluorescent emitting material which can be used for the emitting layer, tetracene derivatives, diamine derivatives and the like can be used.

As a blue phosphorescent emitting material which can be used for the emitting layer, metal complexes such as iridium complexes, osmium complexes and platinum complexes are used. As a green phosphorescent emitting material which can be used for the emitting layer, iridium complexes and the like are used. As a red phosphorescent emitting material which can be used for the emitting layer, metal complexes such as iridium complexes, platinum complexes, terbium complexes and europium complexes are used.

### (Host Material for Emitting Layer)

The emitting layer may have a constitution in which the substance having high emitting property (guest material) is dispersed in another substance (host material). As a substance for dispersing the substance having high emitting property, a variety of substances can be used in addition to the material (the compound represented by the formula (10)) used in the present invention described above, and it is preferable to use a substance having a higher lowest unoccupied molecular orbital level (LUMO level) and a lower highest occupied molecular orbital level (HOMO level) than a substance having high emitting property.

As a substance (host material) for dispersing the substance having high emitting property, 1) a metal complex such as an aluminum complex, a beryllium complex, and a zinc complex, 2) a heterocyclic compound such as an oxadiazole derivative, a benzimidazole derivative, and a phenanthroline derivative, 3) a fused aromatic compound such as a carbazole derivative, an anthracene derivative, a phenanthrene derivative, a pyrene derivative, a naphthacene derivative, a fluoranthene derivative, a triphenylene derivative, a fluorene derivative, and a chrysene derivative, and 4) an aromatic amine compound such as a triarylamine derivative and a fused polycyclic aromatic amine derivative are used.

A compound having delayed fluorescence (thermally activated delayed fluorescence) can also be used as the host material. It is also preferable that the emitting layer include the material used in the present invention described above and the host compound having delayed fluorescence.

### (Electron-Transporting Layer)

The electron-transporting layer is a layer containing a substance having high electron-transporting property. For the electron-transporting layer, 1) a metal complex such as an aluminum complex, a beryllium complex, and a zinc complex; 2) a heteroaromatic compound such as an imidazole derivative, a benzimidazole derivative, an azine derivative, carbazole derivative, and a phenanthroline derivative; and 3) a polymer compound can be used.

### (Electron-Injecting Layer)

The electron-injecting layer is a layer containing a substance having high electron-injecting property. For the electron-injecting layer, lithium (Li), ytterbium (Yb), lithium fluoride (LiF), cesium fluoride (CsF), calcium fluoride (CaF₂), a metal complex compound such as 8-hydroxyquinolinolato-lithium (Liq), an alkali metal such as lithium oxide (LiOₓ), an alkaline earth metal, or a compound thereof can be used.

### (Cathode)

For the cathode, metals, alloys, electrically conductive compounds, mixtures thereof, and the like, which have small work function (specifically 3.8 eV or less) are preferably used. Specific examples of such a cathode material include an element belonging to Group 1 or Group 2 of the Periodic Table of the Elements, i.e., an alkali metal such as lithium (Li) and cesium (Cs), an alkaline earth metal such as magnesium (Mg), calcium (Ca) and strontium (Sr), and an alloy containing these (e.g., MgAg and AILi); a rare earth metal such as europium (Eu) and ytterbium (Yb), and an alloy containing these.

In the organic EL device according to an aspect of the present invention, the method for forming each layer is not particularly limited. A conventionally-known method for forming each layer such as a vacuum deposition process and a spin coating process can be used. Each layer such as the emitting layer can be formed by a known method such as a vacuum deposition process, a molecular beam deposition process (MBE process), or an application process such as a dipping process, a spin coating process, a casting process, a bar coating process and a roll coating process, using a solution prepared by dissolving the material in a solvent.

In the organic EL device according to an aspect of the present invention, the thickness of each layer is not particularly limited, but is normally preferable several nm to 1 μm generally in order to suppress defects such as pinholes, to suppress applied voltages to be low, and to improve luminous efficiency.

### [Electronic Apparatus]

An electronic apparatus according to an aspect of the present invention is characterized by including the organic EL device according to an aspect of the present invention.

Specific examples of the electronic apparatus include display components such as an organic EL panel module; display devices for a television, a cellular μhone and a μersonal computer; and emitting devices such as a light and a vehicular lamp; and the like.

### EXAMPLES

Hereinafter, Examples according to the present invention will be described. The present invention is not limited to these Examples.

### <Compound>

Compounds represented by the formula (1) used in Examples are shown below.

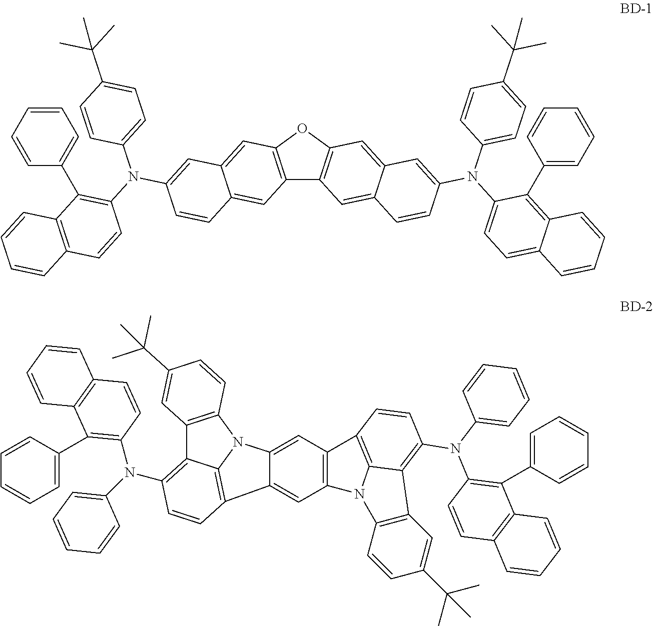 [SMILES: CC(C)(C)c1ccc(N(c2ccc3cc4c(cc3c2)oc2cc3cc(N(c5ccc(C(C)(C)C)cc5)c5ccc6ccccc6c5-c5ccccc5)ccc3cc24)c2ccc3ccccc3c2-c2ccccc2)cc1 ; CC(C)(C)c1ccc2c(c1)c1c(N(c3ccccc3)c3ccc4ccccc4c3-c3ccccc3)ccc3c4cc5c(cc4n2c31)c1ccc(N(c2ccccc2)c2ccc3ccccc3c2-c2ccccc2)c2c3cc(C(C)(C)C)ccc3n5c12]

Comparative compounds used in Comparative Examples are shown below.

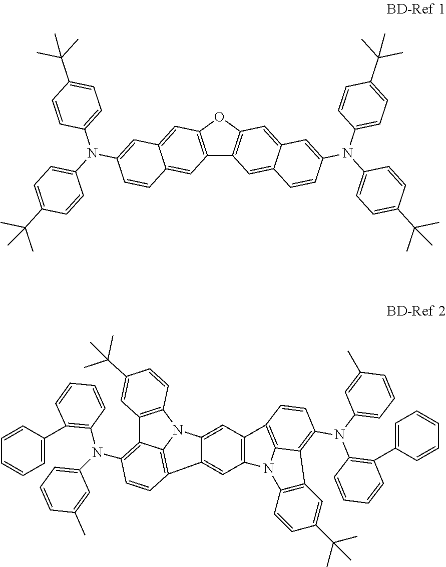 [SMILES: CC(C)(C)c1ccc(N(c2ccc(C(C)(C)C)cc2)c2ccc3cc4c(cc3c2)oc2cc3cc(N(c5ccc(C(C)(C)C)cc5)c5ccc(C(C)(C)C)cc5)ccc3cc24)cc1 ; Cc1cccc(N(c2ccccc2-c2ccccc2)c2ccc3c4cc5c(cc4n4c6ccc(C(C)(C)C)cc6c2c34)c2ccc(N(c3cccc(C)c3)c3ccccc3-c3ccccc3)c3c4cc(C(C)(C)C)ccc4n5c23)c1]

Other compounds used in Example 1 and Comparative Example 1 are shown below.

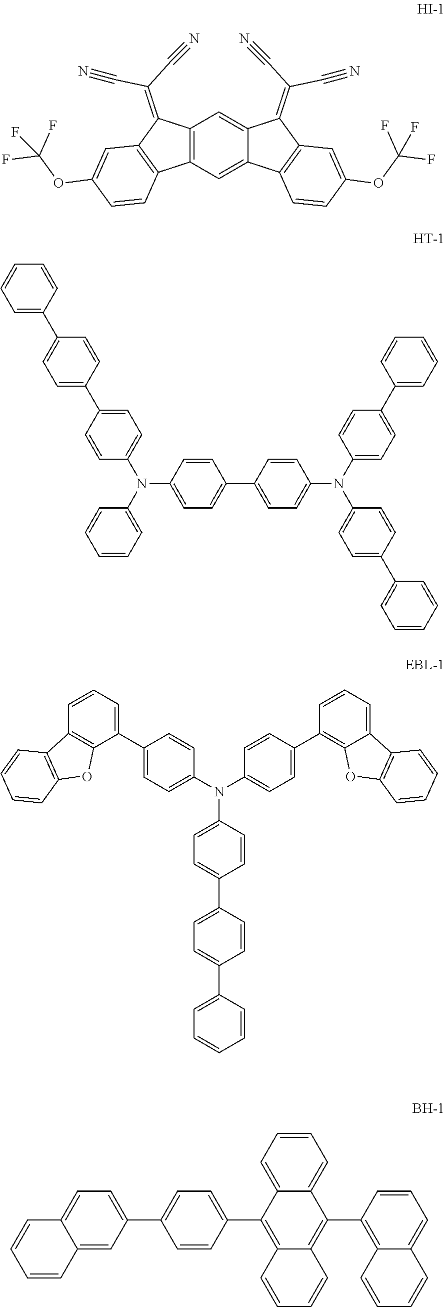

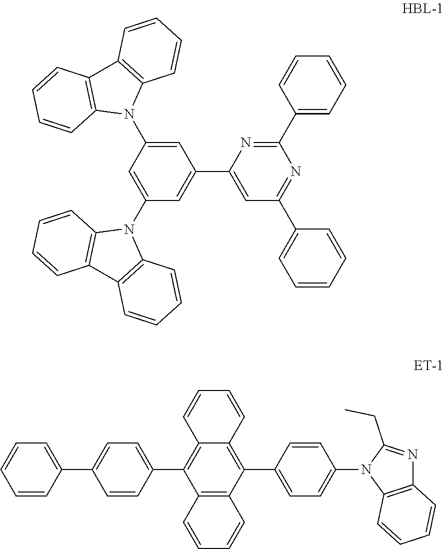 [SMILES: c1ccc(-c2cc(-c3cc(-n4c5ccccc5c5ccccc54)cc(-n4c5ccccc5c5ccccc54)c3)nc(-c3ccccc3)n2)cc1 ; CCc1nc2ccccc2n1-c1ccc(-c2c3ccccc3c(-c3ccc(-c4ccccc4)cc3)c3ccccc23)cc1]

Other compounds used in Example 2 and Comparative Example 2 are shown below.

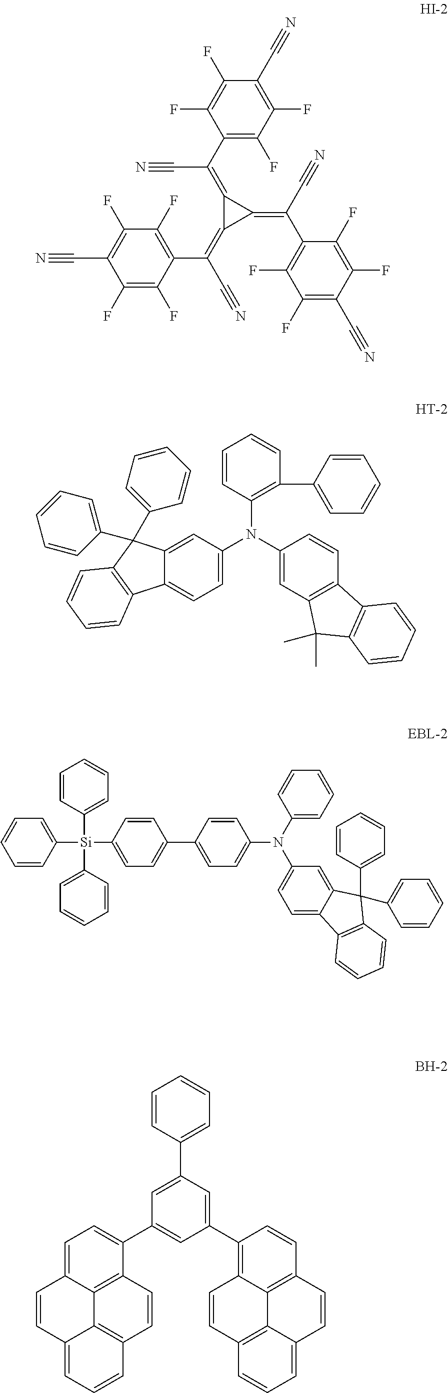

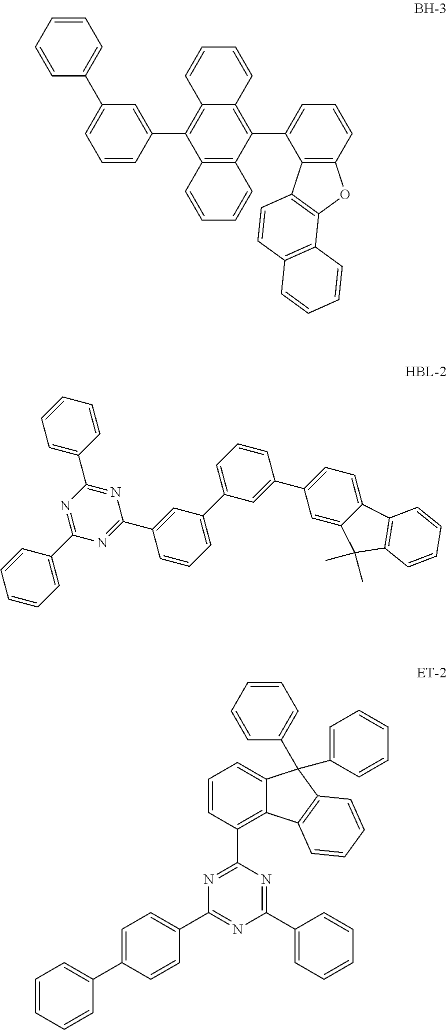 [SMILES: c1ccc(-c2cccc(-c3c4ccccc4c(-c4cccc5oc6c7ccccc7ccc6c45)c4ccccc34)c2)cc1 ; CC1(C)c2ccccc2-c2ccc(-c3cccc(-c4cccc(-c5nc(-c6ccccc6)nc(-c6ccccc6)n5)c4)c3)cc21 ; c1ccc(-c2ccc(-c3nc(-c4ccccc4)nc(-c4cccc5c4-c4ccccc4C5(c4ccccc4)c4ccccc4)n3)cc2)cc1]

### Example 1

<Fabrication of Organic EL Device>

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The ITO has the film thickness of 130 nm.

The glass substrate with the transparent electrode after being cleaned was mounted onto a substrate holder in a vacuum vapor deposition apparatus. First, a compound HI-1 was deposited on the surface on the side where the transparent electrode was formed so as to cover the transparent electrode to form a compound HI-1 film having the thickness of 5 nm. The HI-1 film functions as a hole-injecting layer.

Subsequent to the formation of the HI-1 film, a compound HT-1 was deposited thereon to form an HT-1 film having the thickness of 80 nm on the HI-1 film. The HT-1 film functions as a first hole-transporting layer.

Following the formation of the HT-1 film, a compound EBL-1 was deposited thereon to form an EBL-1 film having the thickness of 10 nm on the HT-1 film. The EBL-1 film functions as a second hole-transporting layer.

A BH-1 (host material) and a BD-1 (dopant material) were co-deposited on the EBL-1 film to be 2% in a proportion (weight ratio) of the compound BD-1 to form an emitting layer having the thickness of 25 nm.

A compound HBL-1 was deposited on the emitting layer to form an electron-transporting layer having the thickness of 10 nm. A compound ET-1 being an electron-injecting material was deposited on the electron-transporting layer to form an electron-injecting layer having the thickness of 15 nm. LiF was deposited on the electron-injecting layer to form a LiF film having the thickness of 1 nm. Metal Al was deposited on the LiF film to form a metal cathode having the thickness of 80 nm.

The device configuration of the organic EL device of Example 1 is schematically shown as follows.
ITO(130)/HI-1(5)/HT-1(80)/EBL-1(10)/BH-1:BD-1(25:2%)/HBL-1(10)/ET-1(15)/LiF(1)/AI(80)

The numerical values in parentheses indicate the film thickness (unit: nm).

### <Evaluation of Organic EL Device>

(Device lifetime)

Regarding the obtained organic EL device, a voltage was applied to the obtained organic EL device at room temperature so that the current density became 50 mA/cm², and the time until the luminance became 95% of the initial luminance (LT95 (unit: hours)) was measured. The results are shown in Table 1. The numerical values in the table are relative values when Comparative Example 1 described later is 100%.

### (Emission Peak Wavelength)

A voltage was applied to the device so that the current density became 10 mA/cm², and at that time, the spectral emission luminance spectrum was measured by using Spectroradiometer CS-2000 (manufactured by KONICA MINOLTA, INC.).

The spectral emission luminance spectrum obtained above was used to obtain an emission peak wavelength. The results are shown in Table 1.

### (Half Width)

The spectral emission luminance spectrum obtained above was used to calculate the half width of the emission peak. The results are shown in Table 1. The numerical values in the Table are relative values when Comparative Example 1 described later is 100%.

### (Second Peak Intensity)

The spectral emission luminance spectrum obtained above was used to calculate the intensity at the wavelength obtained by subtracting the energy of 1450 cm⁻¹ from the emission peak wavelength. The results are shown in Table 1. The numerical values in the Table are relative values when Comparative Example 1 described later is 100%.

### (CIE Chromaticity Coordinate)

A voltage was applied to the device so that the current density became 10 mA/cm², and at that time, CIE1931 chromaticity coordinates (x, y) was measured by using Spectroradiometer CS-2000 (manufactured by KONICA MINOLTA, INC.). The results are shown in Table 1.

### Comparative Example 1

An organic EL device was fabricated and evaluated in the same manner as in Example 1, except that BD-Ref1 was used as the dopant material for the emitting layer. The results are shown in Table 1.

TABLE 1

| | | | LT95 | Emission | | Second | |
|---|---|---|---|---|---|---|---|
| | | | relative | peak | Half | peak | CIE |
| | | | value | wavelength | width | intensity | chromaticity |
| | BH | BD | (%) | (nm) | (%) | (%) | coordinate |
| Example 1 | BH-1 | BD-1 | 150 | 459 | 86 | 91 | (0.136, 0.106) |
| Comparative | | BD-Ref1 | 100 | 461 | 100 | 100 | (0.134, 0.122) |
| Example 1 | | | | | | | |

As seen from the results shown in Table 1, it was found that the device of Example 1 in which the compound BD-1 was used as the dopant material for the emitting layer exhibits high color purity and long lifetime with approximately the same emission peak wavelength as compared to the device of Comparative Example 1.

### Example 2

### <Fabrication of Organic EL Device>

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The ITO has the film thickness of 130 nm.

The glass substrate with the transparent electrode after being cleaned was mounted onto a substrate holder in a vacuum vapor deposition apparatus. First, an HT-2 and an HI-2 were co-deposited on the surface on the side where the transparent electrode was formed so as to cover the transparent electrode to be 10% in a proportion (weight ratio) of the compound HI-2 to form a compounds HT-2:HI-2 film having the thickness of 10 nm. The HT-2:HI-2 film functions as a hole-injecting layer.

Subsequent to the formation of the HT-2:HI-2 film, the compound HT-2 was deposited thereon to form an HT-2 film having the thickness of 85 nm on the HT-2:HI-2 film. The HT-2 film functions as a first hole-transporting layer.

Following the formation of the HT-2 film, a compound EBL-2 was deposited thereon to form an EBL-2 film having the thickness of 5 nm on the HT-2 film. The EBL-2 film functions as a second hole-transporting layer.

A BH-2 (host material) and a BD-2 (dopant material) were co-deposited on the EBL-2 film to be 1% in a proportion (weight ratio) of the compound BD-2 to form a second emitting layer having the thickness of 5 nm.

A BH-3 (host material) and a BD-2 (dopant material) were co-deposited on the second emitting layer to be 1% in a proportion (weight ratio) of the compound BD-2 to form a first emitting layer having the thickness of 15 nm.

A compound HBL-2 was deposited on the first emitting layer to form a first electron-transporting layer having the thickness of 5 nm. A compound ET-2 and an Liq were co-deposited on the first electron-transporting layer to be 50% in a proportion (weight ratio) of the Liq to form a second electron-transporting layer having the thickness of 25 nm.

Yb was deposited on the second electron-transporting layer to form an electron-injecting layer having the thickness of 1 nm. A metal Al was deposited on the Yb film to form a metal cathode having the thickness of 80 nm.

The device configuration of the organic EL device of Example 2 is schematically shown as follows.

ITO(130)/HT-2:HI-2(10:10%)/HT-2(85)/EBL-2(5)/BH-2:BD-2(5:1%)/BH-3:BD-2(15:1%)/HBL-2(5)/ET-2:Liq(25:50%)/Yb(1)/AI(80)

The numerical values in parentheses indicate the film thickness (unit: nm).

### <Evaluation of Organic EL Device>

Regarding the obtained organic EL device, the device lifetime, the emission peak wavelength, the half width, the second peak intensity, and CIE chromaticity coordinate were measured in the same manner as in Example 1.

The results are shown in Table 2. Here, the numerical values of the Table in the device lifetime, the half width and the second peak intensity are relative values when Comparative Example 2 described later is 100%.

### Comparative Example 2

An organic EL device was fabricated and evaluated in the same manner as in Example 2, except that BD-Ref2 was used as the dopant material for the first emitting layer and the second emitting layer instead of the BD-2. The results are shown in Table 2.

TABLE 2

| | | | LT95 | Emission | | Second | |
|---|---|---|---|---|---|---|---|
| | First | | relative | peak | Half | peak | CIE |
| | emitting layer | | value | wavelength | width | intensity | chromaticity |
| | BH | BD | (%) | (nm) | (%) | (%) | coordinate |
| Example 2 | BH-3 | BD-2 | 121 | 455 | 100 | 97 | (0.140,0.083) |
| Comparative | | BD-Ref2 | 100 | 452 | 100 | 100 | (0.143,0.076) |
| Example 2 | | | | | | | |

As seen from the results shown in Table 2, it was found that the device of Example 2 in which the compound BD-2 was used as the dopant material for the first emitting layer exhibits high color purity and long lifetime with approximately the same emission peak wavelength as compared to the device of Comparative Example 2.

### <Synthesis of Compound>

### Synthesis of BD-1

The compound BD-1 was synthesized through the synthetic route described below.

### Synthesis of Intermediate a

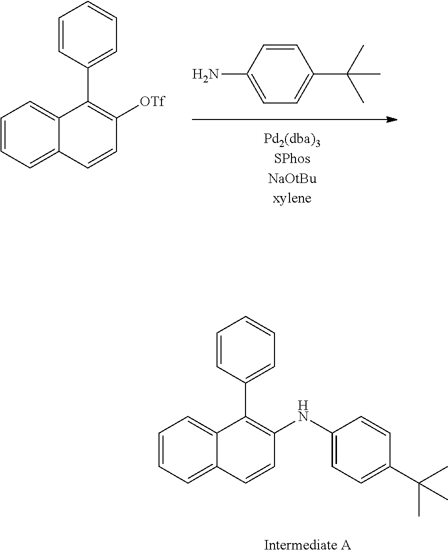 [SMILES: O=S(=O)(Oc1ccc2ccccc2c1-c1ccccc1)C(F)(F)F ; CC(C)(C)c1ccc(N)cc1 ; CC(C)(C)c1ccc(Nc2ccc3ccccc3c2-c2ccccc2)cc1]

1-phenylnaphthalenyl-2-trifluoromethanesulfonate (synthesized by using the method described in Chem. Commun., 2019, 55, 9267) (5.9 g), 4-tert-butylaniline (manufactured by Tokyo Chemical Industry Co., Ltd.) (4.0 mL), tris(dibenzylideneacetone) dipalladium (0) (manufactured by Sigma-Aldrich Co. LLC) (46 mg), 2-dicyclohexylphosphino-2′,6′-dimethoxybiphenyl (Sphos) (manufactured by Sigma-Aldrich Co. LLC) (140 mg), sodium butoxide (2.4 g) and xylene (100 mL) were added in 300 mL of three-necked eggplant flask, and they were stirred at 55° C. for 5 hours under an argon atmosphere. The reaction solution was cooled to room temperature, and then silica gel column chromatography was conducted to obtain a brown solid (3.2 g, 54% yield). The molecular weight of Intermediate A was 351, and the mass spectrum of the obtained compound was analyzed as m/z (ratio of mass to charge)=351, thereby identifying the compound as Intermediate A.

### —Synthesis of BD-1

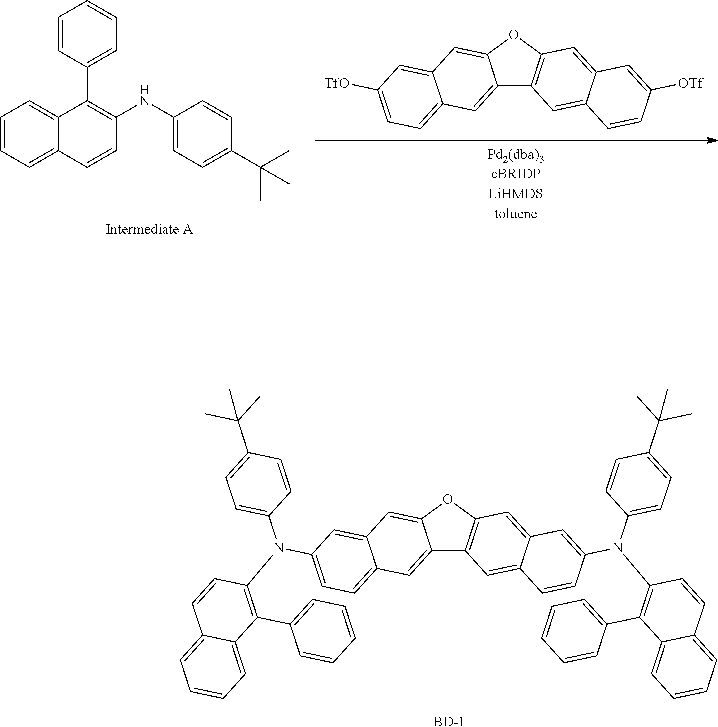 [SMILES: CC(C)(C)c1ccc(Nc2ccc3ccccc3c2-c2ccccc2)cc1 ; O=S(=O)(Oc1ccc2cc3c(cc2c1)oc1cc2cc(OS(=O)(=O)C(F)(F)F)ccc2cc13)C(F)(F)F ; CC(C)(C)c1ccc(N(c2ccc3cc4c(cc3c2)oc2cc3cc(N(c5ccc(C(C)(C)C)cc5)c5ccc6ccccc6c5-c5ccccc5)ccc3cc24)c2ccc3ccccc3c2-c2ccccc2)cc1 ; "BD-1"]

1,1′-dinaphtho[2,3-b:2′,3′-d]furan-3,9-bistrifluoromethanesulfonate (synthesized according to Example 1 of WO 2018/235953 A1) (2.2 g), Intermediate A (3.0 g), tris(dibenzylideneacetone) dipalladium (0) (Pd₂(dba)₃) (71 mg), and di-tert-butyl(1-methyl-2,2-diphenylcyclopropyl) phosphine (0.11 g) were added in a three-necked eggplant flask, and dehydrated xylene (80 mL) was added thereto. The solution was heated to 70° C. under an argon atmosphere and stirred for 30 minutes, and 10 mL of a toluene solution of lithium (bistrimethylsilyl) amide (LiHMDS) (1 mol/L) was added dropwise into the system, followed by stirring for 6 hours. The solution was allowed to cool to room temperature, and then subjected to silica gel column chromatography to obtain a yellow solid. The yield was 1.5 g (40% yield). The molecular weight of BD-1 was 967, and the mass spectrum of the obtained compound was analyzed as m/z (ratio of mass to charge)=967, thereby identified as BD-1.

### Synthesis of BD-2

The compound BD-2 was synthesized through the synthetic route described below.

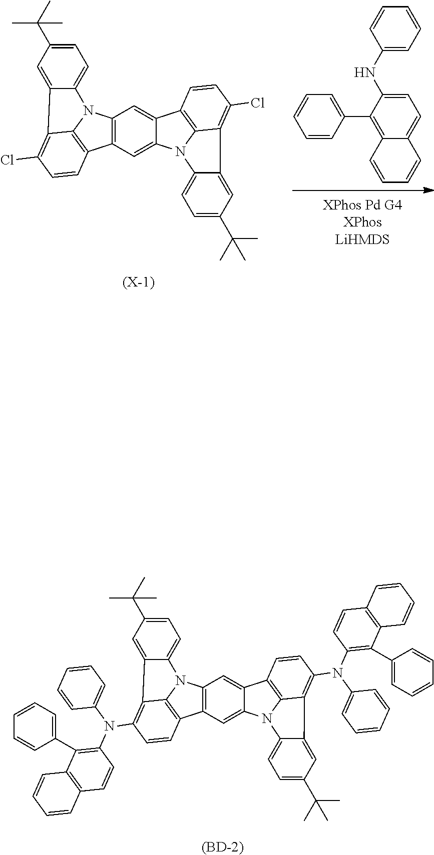

Intermediate X-1 prepared by using the method described in WO 2020/250961 A1 (4.7 g), N-1-diphenyl-2-naphthaleneamine (5.93 g), 2-dicyclohexylphosphino-2′,4′,6′-triisopropylbiphenyl (XPhos) (383 mg), and XPhos Pd G4 (363 mg) were added to toluene (380 mL), and the temperature thereof was increased to 100° C. Atoluene solution of lithium bis(trimethylsilyl) amide (LiHMDS) (1 M, 16.86 mL) was added dropwise into the suspended solution, and it was stirred at 100° C. for two hours under an argon atmosphere. The reaction mixture was naturally cooled to room temperature, and then the generated solid was collected by filtration, followed by being washed with toluene, water, and methanol to obtain a compound BD-2 (3.83g, 43% yield). The molecular weight of the compound BD-2 was 1102.5, and the mass spectrum of the obtained compound was analyzed as m/e=1102.1.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification and the specification of Japanese application(s) on the basis of which the present application claims Paris convention priority are incorporated herein by reference in its entirety.

1. A compound represented by the following formula (1): 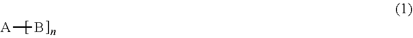
wherein in the formula (1),
A is
a substituted or unsubstituted aromatic hydrocarbon ring group containing a nitrogen atom as a part of a substituent and having a fused ring composed of 5 or more rings, or
a substituted or unsubstituted heterocyclic group containing a nitrogen atom as a part of the skeleton or a substituent and having a fused ring composed of 5 or more rings;
B is a monovalent group represented by the following formula (b1): 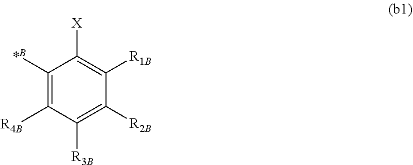
wherein in the formula (b1),
*B is bonded with the nitrogen atom contained as a part of the skeleton of A or a substituent via a single bond;
one or more sets of the adjacent two or more of R_{1B} to R_{4B} form an unsubstituted saturated ring, or a substituted or unsubstituted, unsaturated ring by bonding with each other;
R_{1B} to R_{4B} which do not form the unsubstituted saturated ring, or the substituted or unsubstituted, unsaturated ring are independently a hydrogen atom or a substituent R;
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—S₁(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms;
when two or more substituents R are present, the two or more substituents R may be the same as or different from each other;
X is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—S₁(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group, and
a substituted or unsubstituted aryl group having 6 to 50 ring carbon atoms;
R₉₀₁ to R₉₀₇ are the same as defined in the substituent R; and
n is an integer of 1 to 5; when n is 2 or more, two or more B's may be the same as or different from each other.

2. The compound according to claim 1, wherein n is 1 or 2.

3. The compound according to claim 1, wherein A is a group selected from groups represented by the following formulas (a-1) to (a-9): 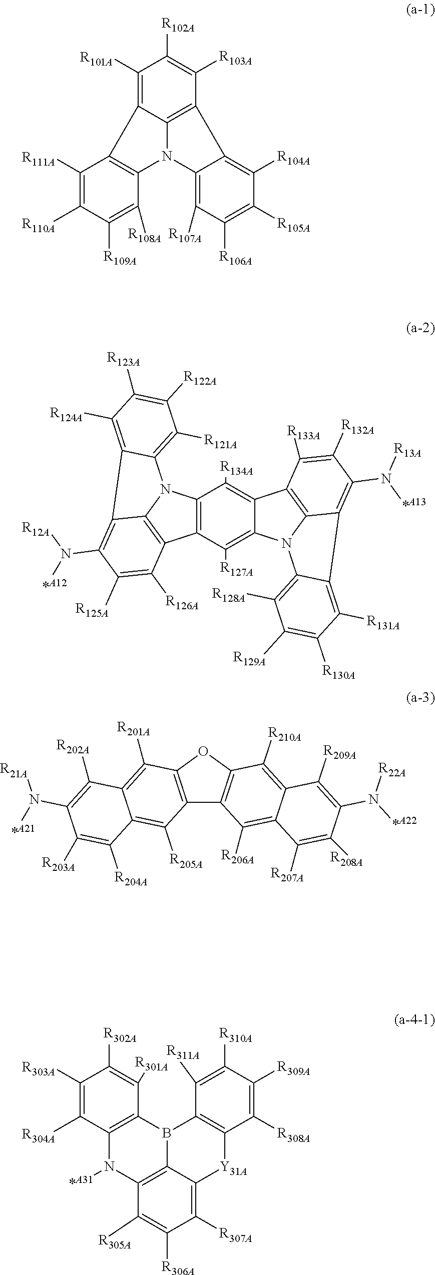 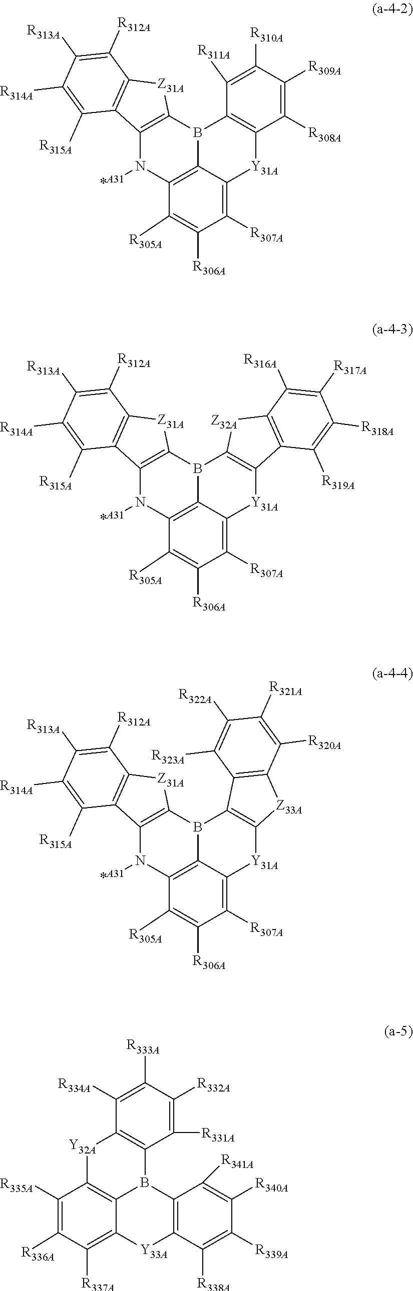 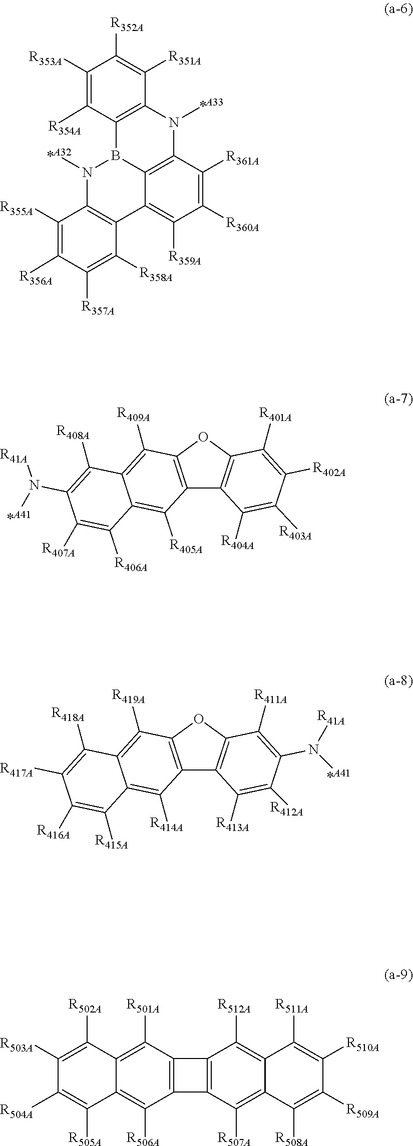
wherein in the formula (a-1),
at least one of R_{101A} to R_{111A} is a group represented by the following formula (a-11); 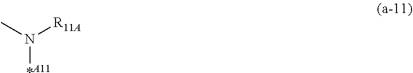
wherein in the formula (a-11),
*A11 is a bonding position in which *B in the formula (b1) is bonded therewith;
R_{11A} is a substituent R;
R_{101A} to R_{111A} which are not the group represented by the formula (a-11) are independently a hydrogen atom or a substituent R;
when two or more groups represented by the formula (a-11) are present, the two or more groups represented by the formula (a-11) may be the same as or different from each other;
in the formula (a-2),
at least one of *A12 and *A13 is a bonding position in which *B in the formula (b1) is bonded therewith;
*A12 and *A13 which is not the bonding position in which *B in the formula (b1) is bonded therewith is a substituent R;
R_{12A} and R_{13A} are a substituent R;
R_{121A} to R_{134A} are independently a hydrogen atom or a substituent R;
in the formula (a-3),
at least one of *A21 and *A22 is a bonding position in which *B in the formula (b1) is bonded therewith;
*A21 and *A22 which is not the bonding position in which *B in the formula (b1) is bonded therewith is a substituent R;
R_{21A} and R_{22A} are a substituent R;
R_{201A} to R_{210A} are independently a hydrogen atom or a substituent R;
in the formula (a-4-1),
*A31 is a bonding position in which *B in the formula (b1) is bonded therewith;
R_{301A} to R_{311A} are independently a hydrogen atom or a substituent R;
Y_{31A} is C(R₁₁)(R₁₂), N(R₁₃), S₁(R₁₄)(R₁₅), O, or S;
R₁₁ to R₁₅ are independently a hydrogen atom or a substituent R;
in the formula (a-4-2),
*A31 is a bonding position in which *B in the formula (b1) is bonded therewith;
R_{305A} to R_{315A} are independently a hydrogen atom or a substituent R;
Y_{31A} is C(R₁₁)(R₁₂), N(R₁₃), S₁(R₁₄)(R₁₅), O, or S;
Z_{31A} is C(R₁₁)(R₁₂), O, or S;
R₁₁ to R₁₅ are independently a hydrogen atom or a substituent R;
when two or more of each of R₁₁ and R₁₂ are present, the two or more of each of R₁₁ and R₁₂ may be the same as or different from each other;
in the formula (a-4-3),
*A31 is a bonding position in which *B in the formula (b1) is bonded therewith;
R_{305A} to R_{307A} and R_{312A} to R_{319A} are independently a hydrogen atom or a substituent R;
Y_{31A} is C(R₁₁)(R₁₂), N(R₁₃), S₁(R₁₄)(R₁₅), O, or S;
Z_{31A} and Z_{32A} are independently C(R₁₁)(R₁₂), O, or S;
R₁₁ to R₁₅ are independently a hydrogen atom or a substituent R;
when two or more of each of R₁₁ and R₁₂ are present, the two or more of each of R₁₁ and R₁₂ may be the same as or different from each other;
in the formula (a-4-4),
*A31 is a bonding position in which *B in the formula (b1) is bonded therewith;
R_{305A} to R_{307A}, R_{312A} to R_{315A}, and R_{320A}to R_{323A} are independently a hydrogen atom or a substituent R;
Y_{31A} is C(R₁₁)(R₁₂), N(R₁₃), S₁(R₁₄)(R₁₅), O, or S;
Z_{31A} and Z_{33A} are independently C(R₁₁)(R₁₂), O, or S;
R₁₁ to R₁₅ are independently a hydrogen atom or a substituent R;
when two or more of each of R₁₁ and R₁₂ are present, the two or more of each of R₁₁ and R₁₂ may be the same as or different from each other;
in the formula (a-5),
at least one of R_{331A} to R_{341A} is the group represented by the formula (a-11);
R_{331A} to R_{341A} which are not the group represented by the formula (a-11) are independently a hydrogen atom or a substituent R;
when two or more groups represented by the formula (a-11) are present, the two or more groups represented by the formula (a-11) may be the same as or different from each other;
Y_{32A} and Y_{33A} are independently C(R₁₁)(R₁₂), N(R₁₃), S₁(R₁₄)(R₁₅), O, or S;
R₁₁ to R₁₅ are independently a hydrogen atom or a substituent R;
when two or more of each of R₁₁ to R₁₅ are present, the two or more of each of R₁₁ to R₁₅ may be the same as or different from each other;
in the formula (a-6),
at least one of *A32 and *A33 is a bonding position in which *B in the formula (b1) is bonded therewith;
*A32 and *A33 which is not the bonding position in which *B in the formula (b1) is bonded therewith is a substituent R;
R_{351A} to R_{361A} are independently a hydrogen atom or a substituent R;
in the formula (a-7),
*A41 is a bonding position in which *B in the formula (b1) is bonded therewith, or a substituent R;
R_{41A} is a substituent R;
the adjacent two of R_{401A} to R_{404A} form a group represented by the following formula (a-12) or (a-13) by bonding with each other: 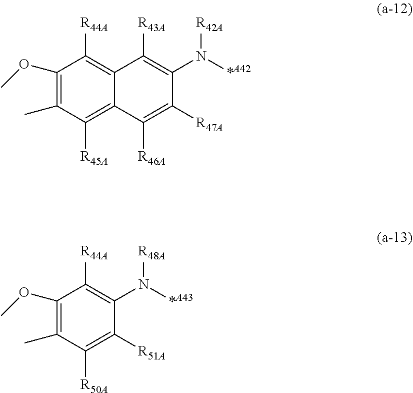
wherein in the formula (a-12),
*A42 is a bonding position in which *B in the formula (b1) is bonded therewith, or a substituent R;
R_{42A} is a substituent R;
R_{43A} to R_{47A} are independently a hydrogen atom or a substituent R;
in the formula (a-13),
*A43 is a bonding position in which *B in the formula (b1) is bonded therewith, or a substituent R;
R_{48A} is a substituent R;
R_{49A} to R₅₁A are independently a hydrogen atom or a substituent R;
when the adjacent two of R_{401A} to R_{404A} form the group represented by the formula (a-12) by bonding with each other, at least one of *A41 and *A42 is a bonding position in which *B in the formula (b1) is bonded therewith;
when the adjacent two of R_{401A} to R_{404A} form the group represented by the formula (a-13) by bonding with each other, at least one of *A41 and *A43 is a bonding position in which *B in the formula (b1) is bonded therewith;
R_{405A} to R_{409A}, and R_{401A} to R_{404A} which do not form the group represented by the formula (a-12) or (a-13) are independently a hydrogen atom or a substituent R;
in the formula (a-8),
*A41 is a bonding position in which *B in the formula (b1) is bonded therewith, or a substituent R;
R_{41A} is a substituent R;
the adjacent two of R_{415A} to R_{418A} form the group represented by the formula (a-12) or (a-13) by bonding with each other;
when the adjacent two of R_{415A} to R_{418A} form the group represented by the formula (a-12) by bonding with each other, at least one of *A41 and *A42 is a bonding position in which *B in the formula (b1) is bonded therewith;
when the adjacent two of R_{415A} to R_{418A} form the group represented by the formula (a-13) by bonding with each other, at least one of *A41 and *A43 is a bonding position in which *B in the formula (b1) is bonded therewith;
R_{411A} to R_{414A}, R_{419A}, and R_{415A} to R_{418A} which do not form the group represented by the formula (a-12) or (a-13) are independently a hydrogen atom or a substituent R;
in the formula (a-9),
at least one of R_{501A} to R_{512A} is the group represented by the formula (a-11);
R_{501A} to R_{512A} which are not the group represented by the formula (a-11) are independently a hydrogen atom or a substituent R;
when two or more groups represented by the formula (a-11) are present, the two or more groups represented by the formula (a-11) may be the same as or different from each other; and
the substituent R is the same as defined in the formula (1).

4. The compound according to claim 1, wherein one or more sets of the adjacent two or more of R_{1B} to R_{4B} form, by bonding with each other,
an unsubstituted saturated hydrocarbon ring having 6 to 12 ring carbon atoms,
a substituted or unsubstituted, unsaturated hydrocarbon ring having 6 to 12 ring carbon atoms,
an unsubstituted saturated heterocycle having 5 to 12 ring atoms, or
a substituted or unsubstituted, unsaturated heterocycle having 5 to 12 ring atoms.

5. The compound according to claim 1, wherein B is a group selected from groups represented by the following formulas (b2-1) to (b2-15): 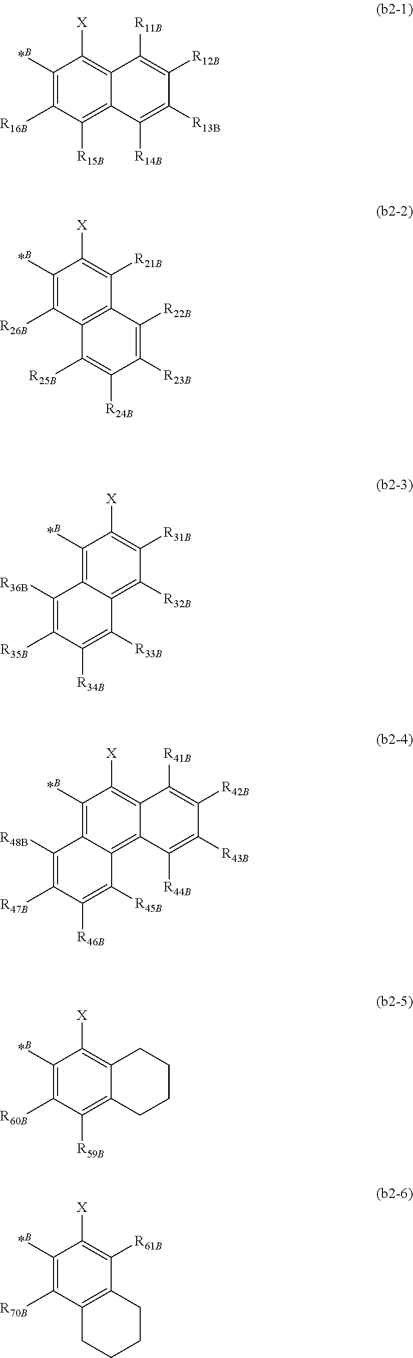 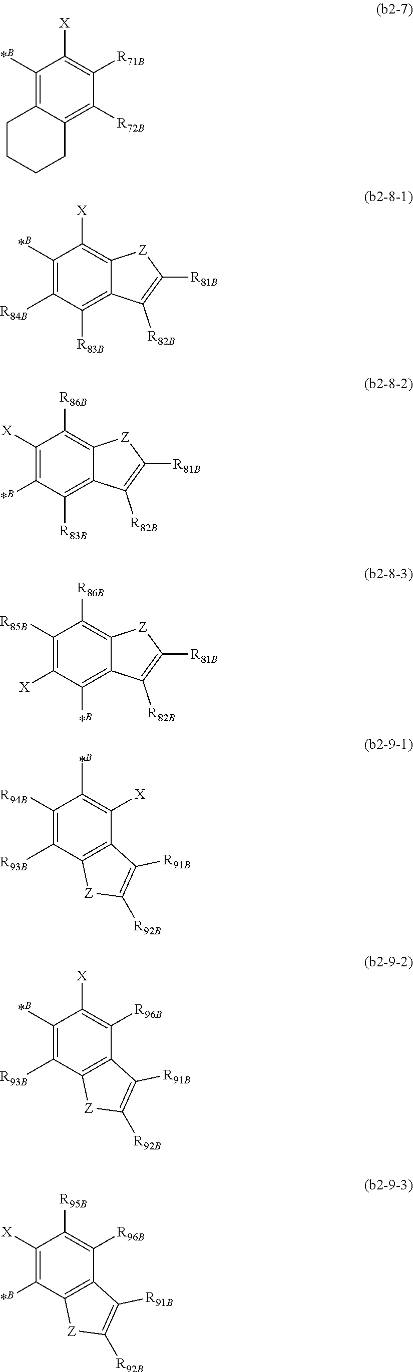 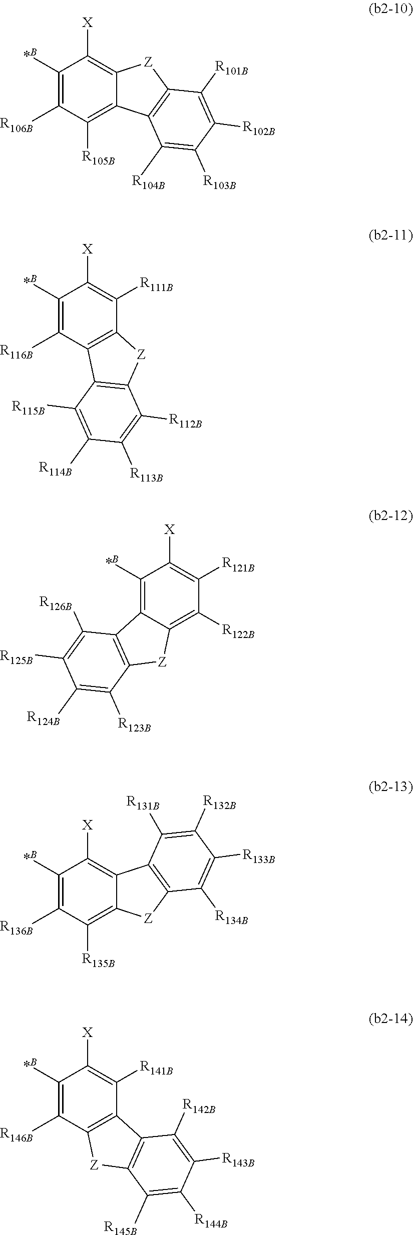 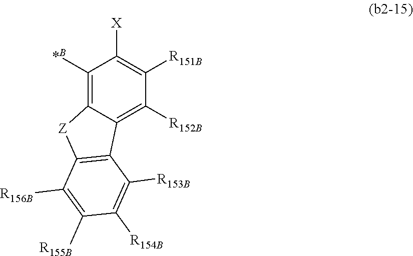
wherein in the formulas (b2-1) to (b2-15),
*B is bonded with the nitrogen atom contained as a part of the skeleton of A or a substituent via a single bond;
R_{11B} to R_{16B}, R_{21B} to R_{26B}, R_{31B} to R_{36B}, R_{41B} to R_{48B}, R_{59B} to R_{60B}, R_{61B}, R_{70B}, R_{71B} to R_{72B}, R_{81B} to R_{86B}, R_{91B} to R_{96B}, R_{101B} to R_{106B}, R_{111B} to R_{116B}, R_{121B} to R_{126B}, R_{131B} to R_{136B}, R_{141B} to R_{146B}, and R_{151B} to R_{156B} are independently a hydrogen atom or a substituent R;
Z is O, S, or C(R₁₁)(R₁₂);
R₁ₗ and R₁₂ are a substituent R; and
X and the substituent R is the same as defined in the formula (1).

6. The compound according to claim 1, which is represented by the following formula (1-1): 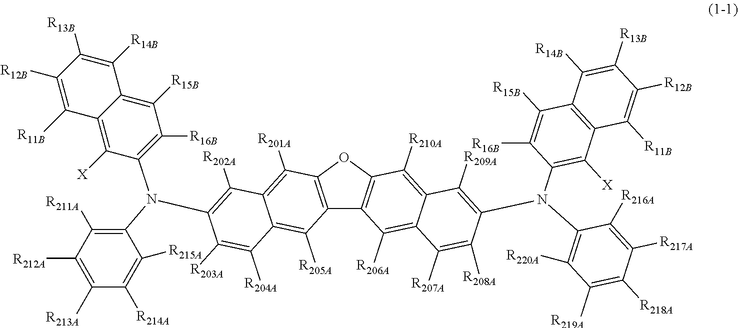
wherein in the formula (1-1),
R_{201A} to R_{220A} and R_{11B} to R_{16B} are independently a hydrogen atom or a substituent R;
X and the substituent R is the same as defined in the formula (1); and
each of the two X's and the two R_{1lB}'s to the two R_{16B}'s may be the same as or different from each other.

7. The compound according to claim 6, which is represented by the following formula (1-2): 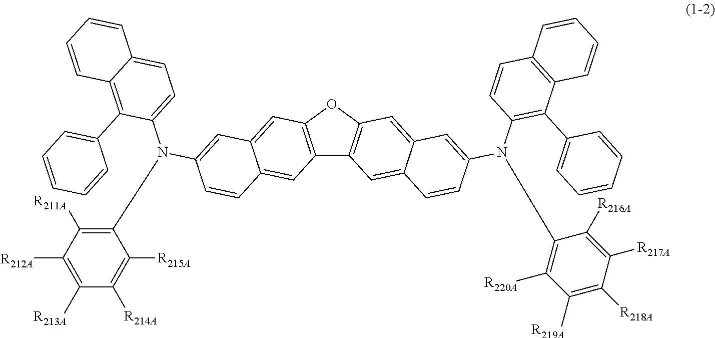
wherein in the formula (1-2),
R_{211A} to R_{220A} are independently a hydrogen atom or a substituent R; and
the substituent R is the same as defined in the formula (1).

8. The compound according to claim 1, which is represented by the following formula (2-1): 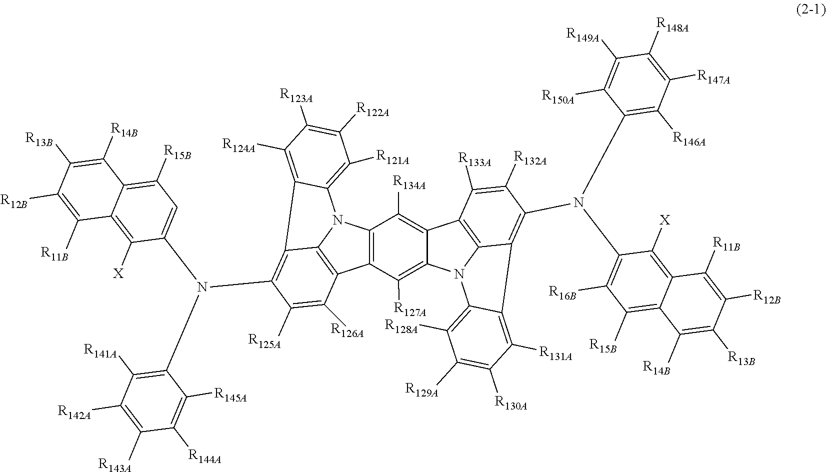
wherein in the formula (2-1),
R_{121A} to R_{134A}, R_{141A} to R_{150A}, and R_{11B} to R_{16B} are independently a hydrogen atom or a substituent R;
X and the substituent R is the same as defined in the formula (1); and
each of the two X's and the two R_{11B}'s to the two R_{16B}'s may be the same as or different from each other.

9. The compound according to claim 8, which is represented by the following formula (2-2): 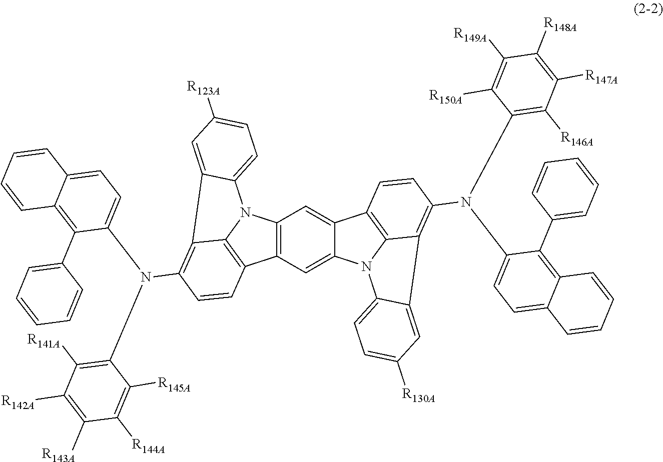
wherein in the formula (2-2),
R_{123A}, R_{130A}, and R_{141A} to R_{150A} are independently a hydrogen atom or a substituent R; and
the substituent R is the same as defined in the formula (1).

10. The compound according to claim 9, wherein R_{123A} and R_{130A} are a substituted or unsubstituted alkyl group having 1 to 5 carbon atoms.

11. The compound according to claim 1, wherein a substituent in the case of “substituted or unsubstituted” is selected from the group consisting of an alkyl group having 1 to 50 carbon atoms, a haloalkyl group having 1 to 50 carbon atoms, an alkenyl group having 2 to 50 carbon atoms, an alkynyl group having 2 to 50 carbon atoms, a cycloalkyl group having 3 to 50 ring carbon atoms, an alkoxy group having 1 to 50 carbon atoms, an alkylthio group having 1 to 50 carbon atoms, an aryloxy group having 6 to 50 ring carbon atoms, an arylthio group having 6 to 50 ring carbon atoms, an aralkyl group having 7 to 50 carbon atoms, —S₁(R₄₁)(R₄₂)(R₄₃), —C(═O)R₄₄, —COOR₄₅, —S(═O)₂R₄₆, —P(═O)(R₄₇)(R₄₈), —Ge(R₄₉)(R₅₀)(R₅₁), —N(R₅₂)(R₅₃) (wherein, R₄₁ to R₅₃ are independently a hydrogen atom, an alkyl group having 1 to 50 carbon atoms, an aryl group having 6 to 50 ring carbon atoms, or a monovalent heterocyclic group having 5 to 50 ring atoms; when two or more of each of R₄₁ to R₅₃ are present, the two or more of each of R₄₁ to R₅₃ may be the same as or different from each other), a hydroxy group, a halogen atom, a cyano group, a nitro group, an aryl group having 6 to 50 ring carbon atoms, and a monovalent heterocyclic group having 5 to 50 ring atoms.

12. A material for an organic electroluminescence device comprising the compound according to claim 1.

13. An organic electroluminescence device comprising
a cathode;
an anode; and
at least one organic layer arranged between the cathode and the anode,
wherein at least one layer of the at least one organic layer comprises the compound according to claim 1.

14. The organic electroluminescence device according to claim 13, wherein the at least one layer of the at least one organic layer comprises a second compound which is not the same as the first compound represented by the following formula (1): 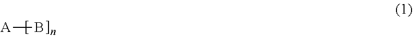
wherein in the formula (1),
A is
a substituted or unsubstituted aromatic hydrocarbon ring group containing a nitrogen atom as a part of a substituent and having a fused ring composed of 5 or more rings, or
a substituted or unsubstituted heterocyclic group containing a nitrogen atom as a part of the skeleton or a substituent and having a fused ring composed of 5 or more rings;
B is a monovalent group represented by the following formula (b1): 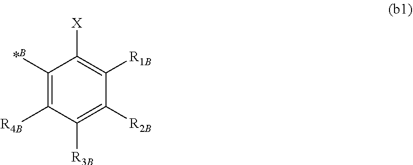
wherein in the formula (b1),
*B is bonded with the nitrogen atom contained as a part of the skeleton of A or a substituent via a single bond;
one or more sets of the adjacent two or more of R_{1B} to R_{4B} form an unsubstituted saturated ring, or a substituted or unsubstituted, unsaturated ring by bonding with each other;
R_{1B} to R_{4B} which do not form the unsubstituted saturated ring, or the substituted or unsubstituted, unsaturated ring are independently a hydrogen atom or a substituent R;
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—S₁(R₉₀1)(R₉₀₂)(R₉₀₃),
—O—(R₉₀₄),
—S—(R₉₀₅),
—N(R₉₀₆)(R₉₀₇)
(wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms: when two or more of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms;
when two or more substituents R are present, the two or more substituents R may be the same as or different from each other;
X is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—S₁(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group, and
a substituted or unsubstituted aryl group having 6 to 50 ring carbon atoms;
R₉₀₁ to R₉₀₇ are the same as defined in the substituent R; and
n is an integer of 1 to 5: when n is 2 or more, two or more B's may be the same as or different from each other.

15. The organic electroluminescence device according to claim 14, wherein the second compound is a heterocyclic compound or a fused aromatic compound.

16. The organic electroluminescence device according to claim 14, wherein the second compound is an anthracene derivative.

17. The organic electroluminescence device according to claim 14, wherein the second compound is a compound represented by the following formula (20): 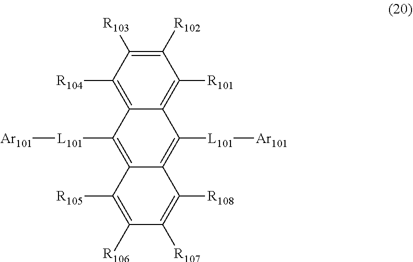
wherein in the formula (20),
one or more sets of the adjacent two or more of R₁₀₁ to R₁₀₈ form a substituted or unsubstituted, saturated or unsaturated ring, or do not form the substituted or unsubstituted, saturated or unsaturated ring;
R₁₀₁ to R₁₀₈ which do not form the substituted or unsubstituted, saturated or unsaturated ring are independently a hydrogen atom or a substituent R;
L₁₀₁'s are
a single bond,
a substituted or unsubstituted arylene group having 6 to 50 ring carbon atoms, or
a substituted or unsubstituted divalent heterocyclic group having 5 to 50 ring atoms;
Ar₁₀₁'s are
a substituted or unsubstituted aryl group having 6 to 50 ring carbon atoms, or
a substituted or unsubstituted monovalent heterocyclic group having 5 to 50 ring atoms;
each of the two L₁₀₁'s and the two Ar₁₀₁'s may be the same as or different from each other; and
the substituent R is the same as defined in the formula (1).

18. The organic electroluminescence device according to claim 13, wherein the at least one layer of the at least one organic layer is an emitting layer.

19. The organic electroluminescence device according to claim 18, wherein a hole-transporting layer is provided between the anode and the emitting layer.

20. The organic electroluminescence device according to claim 18, wherein an electron-transporting layer is provided between the cathode and the emitting layer.

21. The organic electroluminescence device according to claim 18, wherein the emitting layer comprises a compound represented by the formula (20).

22. The organic electroluminescence device according to claim 18, wherein the emitting layer further comprises a host compound having delayed fluorescence.

23. An electronic apparatus comprising the organic electroluminescence device according to claim 13.
